(19) United States
(12) Patent Application Publication
(10) Pub. No.: US 2004/0127406 A1
(43) Pub. Date: Jul. 1, 2004

(54) Methods for in vitro expansion and transdifferentiation of human pancreatic acinar cells into insulin-producing cells

(76) Inventors: Sharon C. Presnell, Raleigh, NC (US); Mohammad Heidaran, Cary, NC (US); Perry Haaland, Chapel Hill, NC (US)

Correspondence Address: VENABLE LLP, 575 7TH STREET, N.W., WASHINGTON, DC, 20004-1601, US

(21) Appl. No.: 10/443,732

(22) Filed: May 22, 2003

Related U.S. Application Data
(60) Provisional application No. 60/384,000, filed on May 28, 2002.

(51) Int. Cl. A61K 38/17; A61K 38/16; C12N 5/08

(52) U.S. Cl. 514/12; 435/366

(57) ABSTRACT

This invention relates, e.g., to a method for expanding mammalian acinar cells, comprising culturing the cells in a cell culture system comprising a cell culture medium and a cell attachment surface, under conditions wherein the acinar cells undergo a 3-4 fold expansion together with transdifferentiation into a modified cell phenotype (IP cells) showing characteristics of acinar cells and liver cells. The invention also relates to a method for transforming these IP cells to insulin-producing cells in vitro, comprising culturing the cells in a novel, defined medium. Also disclosed are suitable culture media for performing these methods, isolated cells having the phenotype of IP cells and/or produced by these methods, and kits for performing the methods.

This application claims the benefit of provisional application 60/384,000, filed May 28, 2002, which disclosure is incorporated by reference in its entirety herein.

### BACKGROUND OF THE INVENTION

1. Field of the Invention

The invention relates to compositions and methods whereby, e.g., human pancreatic acinar cells are cultured under conditions that support expansion and transdifferentiation into glandular epithelial cells and subsequently into insulin-producing cells.

2.Background Information

The potential benefits of taking insulin-producing cells from organ donors and transplanting them into insulin-dependent, Type I diabetic patients is clear. In the Edmonton clinical trials, many patients have lived free from the delivery of exogenous insulin for approximately 2 years after being transplanted with intact islets from organ donor sources. However, current technology requires two organ donor pancreata to generate a sufficient number of islets (about 1 million islets, comprised of about 1,000 cells each) to transplant into one diabetic patient for cellular therapy. Thus, there has been emphasis in the diabetes field to identify new sources of insulin-producing cells for transplantation. Many avenues are being explored, including expansion of islets after harvest and before transplantation and the generation of new islets from stem-like cells derived from the bone marrow, or from precursor cells located in the pancreas. The challenges presented by these approaches are related to maintenance of function of islets over long periods of culture, and of the relative rarity of stem-like cells that can be harnessed for insulin production from the bone marrow and pancreas. The ductular precursor stem-like cells derived from the pancreas are reported to be more efficient than bone-marrow derived cells at differentiation into insulin-producing cells, and this may reflect their site of origin (i.e., pancreas) where they are certainly exposed to many differentiation signals related to the pancreatic microenvironment. The most abundant cell type in the pancreas is the acinar cell, which comprises about 85% of the pancreas. The acinar cells serve to produce and secrete digestive enzymes and, like islet cells, arise during development from the ductular cell compartment.

There have been reports that acinar cells, when cultivated in vitro, especially under conditions of stress, can undergo a ‘transdifferentiation’ into a cell type that resembles ductular cells, as determined by expression of CK19, CK7, and carbonic anhydrase (all reputed by the authors to be markers of duct cells) (Kerr-Conte, 1996; WO 02/29010 A2), Hall et al., 1992). Furthermore, Bouwens et al. (1998) have shown in vivo, in a model of pancreatic duct ligation, that acinar cells in the ligated portion of the pancreas undergo transdifferentiation into cells with a ductular phenotype. Further work has suggested that insulin-producing cells can be produced upon further differentiation of the duct cells in the ligated portion of the pancreas. The acinar cells are also reported to be of limited survivability in primary culture, with some culture conditions leading to loss of at least 50% of cells within a week. While primary duct cells have been demonstrated in vitro to convert into insulin-producing cells under some culture conditions (e.g. Bonner Weir, 2000, U.S. Pat. No. 6,011,647), there are no reports of cells that arose from acinar cells in vitro differentiating further to produce islet-like cells.

Prior to the development of the present system, primary pancreatic acinar cells were expanded without differentiation into insulin-producing cells, either in serum-containing medium (undesirable both because of the risk and the uncertainty associated with the use of serum), or in complex serum free media formulations. Likewise, primary pancreatic acinar cells have been transdifferentiated into insulin-producing cells without expansion, producing cells with an insulin-producing phenotype in small numbers. Furthermore, it has not been previously possible to obtain insulin-producing cells in good numbers using acinar cells as starting material.

Thus, there is a need for a simple cell culture system and method for rapidly generating large numbers of cells that can further differentiate into, e.g., insulin-producing cells, through expansion and transdifferentiation of the abundant pancreatic acinar cells. Further, there is a need for a cell culture system and method for culturing and transforming such cells into insulin-producing cells. One cell culture system and related method disclosed herein allows a simple, one-step approach that generates expanded cultures that contain at least 80% intermediate progenitor cells that can give rise to insulin-producing cells. A second cell culture system and related method allows the further culturing of these intermediate progenitor cells or other glandular epithelial cells to obtain insulin-producing cells. Both IP cells and insulin-producing cells will be useful for cell-based therapies for the treatment of diseases such as diabetes.

### SUMMARY OF THE INVENTION

The present invention provides compositions and methods whereby, e.g., acinar cells can be cultivated successfully in vitro, undergoing a 3-4 fold increase in cell number over time, and giving rise to a cell population that co-expresses acinar and ductal markers early during the culture (2-3 days ex vivo), then ultimately (e.g., about 7-8 days ex vivo) acquires a modified phenotype characterized by expression of some acinar-associated genes, as well as some liver-associated genes. The genes expressed by these modified cells at about 7-8 days ex vivo include, e.g., ductular cytokeratins (CK7, CK8, CK18 and CK19), hepatic nuclear factor 1 (HNF1), alpha-1 antitrypsin, pi-glutathione s transferase (pi-GST), liver-specific (basic helix-loop-helix (bHLH) transcription factor, Thy-1, CCAAT/enhancer-binding protein (C/EBP)-alpha and C/EBP-beta. These cells exhibit little if any expression of the pancreas-associated genes carbonic anhydrase, cystic fibrosis transmembrane conductance regulator (CFTR), elastase and amylase. By “little if any” expression of a gene is meant herein that gene expression is generally undetectable under conventional methods, such as the hybridization and immunocytochemical methods described herein, but expression may be detected by extraordinarily sensitive methods, such as PCR-based analysis. This type of modified cell is referred to herein as an intermediate progenitor (“IP”) cell. The expanded/transdifferentiated acinar cells (IP cells) can be produced using a general serum-containing media, or, in a preferred method, can be produced without serum on a surface comprising one or more extracellular matrix molecules (ECMs) in the presence of one or more soluble active factors. ECMs can be presented in 2 dimensional or 3 dimensional culture systems in the presence of soluble active factors.

The IP cells generated from these cultures are expected to be useful directly in certain medical applications. For example, there is evidence that such cells may under certain conditions become functioning insulin-producing cells when implanted in diabetic patients. The cells can also be used for drug discovery and toxicity studies.

In addition, according to a further aspect of the invention, the IP cells can be cultivated further, in a serum-free medium composed of any standard serum-free base medium (DMEM:HamsF12, for example) with BSA and combinations of factors, including ECMs, small molecules, and growth factors. After 5-10 days of culture, the IP cells undergo additional steps of differentiation, culminating in the formation of cell aggregates that express pro-insulin and C-peptide. Challenge of these cultures with a high-glucose medium causes release of insulin and C-peptide into the medium, indicating the production in these cultures of functional islet-like cells.

Thus, in a first aspect, the present invention provides a cell culture system comprising a superior cell attachment surface that also stimulates cellular expansion, and a simple culture medium including effective amounts of one or more soluble active factors, or serum (e.g. fetal bovine serum), added to a base medium composition. The cell culture system will be particularly useful for primary culture of mammalian epithelial cells, particularly human epithelial cells. In a preferred embodiment the cell culture system is used for the expansion and transdifferentiation of primary acinar cells, especially human pancreatic acinar cells.

The cell attachment surface for this cell culture system is any surface to which the cells can attach and expand, including both 2 dimensional (e.g. plates, flasks, roller bottles, petri dishes, wells etc.) and 3 dimensional (e.g. scaffold) environments. Preferably the surface comprises at least one type of ECM, or a peptide fragment thereof. Cells may, in some circumstances, detach from these surfaces and form self-supporting aggregates. Suitable fragments include peptides consisting of a sequence of three of more amino acid residues that are identical to any portion of the amino acid sequence of the ECM. Such fragments can be easily made and tested by means known to those of skill in the art. Most preferably the surface is a layer of collagen I. Many other surfaces known in the art are also suitable, such as Collagen VI, Collagen IV, Vitronectin, or Fibronectin. Collagen I is preferred due to ease and cost.

The base medium to which the soluble active factors are added may be any cell culture medium appropriate for growth and differentiation of epithelial cells. These include, but are not limited to, DMEM, Hams F12, MEM, M-199 and RPMI. The general requirements for such culture media and many suitable examples are known to those of skill in the art. To this base medium is added either serum (such as fetal bovine serum), or a stabilizing protein such as bovine serum albumin (BSA) along with effective amounts of soluble active factors. The medium is preferably serum-free.

Soluble active factors for the expansion and transdifferentiation of primary pancreatic acinar cells into IP cells include growth factors such as HGF receptor activators and EGF receptor activators. Preferred soluble active factors include one or more of EGF and Transforming Growth Factor-α, IGF1, HGF, betacellulin, prolactin and gastrin 1. HGF, EGF and/or Transforming Growth Factor-α are particularly preferred. Also preferred is the combination of IGF1 and betacellulin.

In one particularly preferred embodiment, the base medium contains a 1:1 mixture of DMEM and Hams F12. The base medium is completed with the addition of glutamine to a final concentration of ˜4 mM, insulin (˜0.1-10 μg/ml, preferably ˜0.01 mg/ml), transferrin (˜0.5-10 μg/ml, preferably ˜0.0055 mg/ml), selenium (˜0.25-5.0 ng/ml, preferably ˜0.0067 μg/ml of sodium selenite), and Epidermal Growth Factor (EGF) (˜1-20 ng/ml, preferably ˜10 ng/ml); this medium is hereafter referred to as pancreatic cell medium, or PCM. To this base medium formulation, up to ˜20% Fetal Bovine Serum (or other serum), preferably between ˜10-˜15% fetal bovine serum, most preferably about 10% or up to about 15% fetal bovine serum) may be added, or, to create a serum-free culture environment, the following components are added in place of serum: heat-inactivated bovine serum albumin (0.1-2%), Hepatocyte growth factor (HGF) (1-20 ng/ml), and/or Transforming Growth Factor Alpha (TGFα)(1-10 ng/ml). In addition, the medium may contain Betacellulin (0.5-20 ng/ml), Gastrin 1 (0.05-10 ng/ml), Prolactin (1.0-10 ng/ml), and/or IGF-1 (5-100 ng/ml). In particular formulations, greater or lesser amounts of these components may be added in order to achieve a formulation that is effective in supporting the expansion and transdifferentiation of the cells. Persons of skill in the art will appreciate that determining effective amounts of the components will require no more than routine experimentation.

By the use of this attachment surface and medium, the expansion and transdifferentiation of primary pancreatic cells with the desired phenotype is simplified greatly.

In a particularly preferred embodiment, the cell culture system is a combination of collagen I coated tissue culture surface (presented in a 2 dimensional or 3 dimensional form) and a serum-free medium containing BSA, insulin, transferrin, selenium, Hepatocyte growth factor (HGF), Epidermal Growth Factor (EGF) and Transforming Growth Factor Alpha (TGFA).

The cell culture system enables superior attachment in vitro of primary pancreatic epithelial cells for adherent culture compared to prior methods, while creating a cellular environment that promotes expansion of the epithelial component of primary pancreatic cultures with concomitant transdifferentiation of the acinar cells present in the starting material into IP cells, while minimizing emergence of undesired fibroblasts. Advantages of this culture system are ease of construction, few components needed, and that all components are readily available and easily used in the required manner.

The components of this aspect of the invention may be conveniently packaged in the form of a kit. The kit may include, for example, 1) a cell culture medium such as DMEM: 2) a serum-free medium supplement containing BSA, insulin, transferrin, selenium, HGF, EGF and TGFA, in suitable amounts to yield the concentrations noted above in the completed medium; and 3) at least one collagen I coated substrate, such as a vessel for tissue culture (e.g., dish(es) with at least one collagen-1 coated tissue culture surface), or collagen-1 coated inserts for use in culture dishes or other laboratory ware. The kit may also optionally include a tissue culture dish or other cell culture accessories and additional reagents that may be required to carry out epithelial cell culture and differentiation.

Culture systems consisting of scaffolds, collagen coated flasks or other vessels and serum-free base medium may be packaged along with the soluble active factors as a separate vial that would be added to the culture medium just prior to use. The active factor combination can be added to a variety of base media to accomplish the same end, e.g., growth and differentiation of primary pancreatic acinar cells in vitro. Such culture systems should also be useful for other cell types, particularly glandular epithelial cells derived from other organs and tissues, including those from liver, pancreas, intestine, prostate, and breast.

The collagen I surface provides superior cell attachment (thereby increasing the number of cells that adhere during initial culture and thus enhancing culture efficiency), while the collagen I and the combination of soluble active factors (e.g., HGF, TGFA and EGF) promote continued proliferation of cells over time, leading to an increase in cell number above what has been previously reported for primary pancreatic acinar cells. Furthermore, the expansion of the acinar cells is accompanied by a transdifferentiation in the majority of cells to an IP phenotype, which is potentially a therapeutically useful cell phenotype for the treatment of diseases such as diabetes. This likely occurs due to convergence of the intracellular signaling pathways associated with collagen I, HGF, TGFA and EGF, creating a synergistic response.

The cell culture system of the present invention has unexpected advantages over systems previously in use. Collagen I, IV, VI, Vitronectin and Fibronectin were expected to enhance cell attachment. However, other extracellular matrix molecules that yielded equivalent attachment of cells during the initial 18 hours of culture did not promote consistent growth of the cells over time in the serum-free medium containing HGF/EGF/TGFA. The most efficient and cost-effective method of achieving cell expansion AND differentiation into IP cells is to utilize a collagen-I surface and a medium containing reduced serum (preferably less than 20%, more preferably less than 15%, 10%, or 5%, most preferably 2%).

Another aspect of the invention is a method for culturing mammalian epithelial cells comprising adding said cells to the cell culture system described above, and maintaining them at suitable temperature and atmospheric conditions. By “mammalian epithelial cell” is meant any cell of a tissue or organ with an epithelial cell phenotype, defined by the presence of expression of cytokeratins and often through the presence of markers that suggest a tissue-specific function (i.e., epithelial cells of the skin make keratin, epithelial cells of the intestine make mucin, epithelial cells of the prostate make PSA). In a preferred embodiment, the cells are primary pancreatic cells, particularly human pancreatic cells. Suitable temperature for mammalian cells is usually in the range of about 37° C., but may be varied somewhat according to cell type. The atmosphere can be ordinary air, or other specialized mixtures of gasses suitable for maintaining cells, as will be familiar to persons of skill in the art. Expansion of pancreatic acinar cells can be maximized by decreasing the oxygen tension in the culture atmosphere to less than 21%, while transdifferentiation to IP cells can be enhanced by increasing oxygen tension to greater than 5%. A preferred range of oxygen tension is between about 5% and about 21%.

In a second aspect, the invention also provides methods and compositions for transforming glandular epithelial cells that have acquired expression of markers characteristic of an intermediate progenitor (IP) phenotype as described above into insulin-producing cells. By “glandular epithelial cell” is meant an epithelial cell that is a component of a gland. Glands are tissues that have a specific function related to secretion of key molecules—most organs in the body have glandular function (liver, intestine, pancreas, prostate, breast, pituitary, adrenal, kidney) whereby they produce and release hormones, digestive enzymes, or other life-essential fluids. Glandular epithelial cells from endoderm-derived organs (e.g., liver, intestine, pancreas) share many characteristics, including the ability to express many of the same genes. Particularly preferred are glandular epithelial cells from pancreas, for example acinar cells. As used herein, the terms “express” and “expression” generally refer to nucleic acids (e.g., mRNAs) or to protein gene products that are detectable by standard immunocytochemical methods.

In this aspect, the invention provides a second cell culture system comprising a cell attachment surface and a culture medium that supports and promotes the transformation of glandular epithelial cells into insulin-producing cells. The cell attachment surface is similar to and may be identical to the attachment surface for expanding primary pancreatic acinar cells. It may be presented in the form of a flat surface coated on a vessel or in the form of a scaffold or other surface adapted for cell culture. It can be comprised of, or coated with, any composition that is capable of maintaining cells or supporting cell growth. In a preferred embodiment, it comprises at least one ECM, such as Collagen I, Collagen VI, Collagen TV, Vitronectin or Fibronectin. In a particularly preferred embodiment, the cell attachment surface is Collagen-I.

In this aspect, the invention provides a further culture medium comprising at least one differentiation promoting factor (“DPF”) that promotes the transformation of glandular epithelial cells into insulin producing cells. The DPFs for the transformation of glandular epithelial cells into insulin producing cells can be one or more of Activin A, acidic FGF, basic FGF, C-Natriuretic Peptide (CNP), Calcitonin Gene Related Peptide, Cholera Toxin B Subunit, Dexamethasone, Gastrin-Releasing Peptide, Glucagon-like Peptide-1 (GLP-1), Glucose, IGF1, IGF2, Insulin, Laminin, LIF, Met-Enkephalin, PDGFAA+PDGFBB, Prolactin, Sonic Hedgehog, Substance P, TGF-alpha, Trolox (alpha-tocopherol derivative), or VEGF. Preferred concentrations in culture medium of each of these 23 DPFs are listed in Table 1. Although in some cases one DPF is sufficient, preferably two or more factors are used. As many as all 23 of the factors may be used.

TABLE 1

| | Concentration |
|---|---|
| | (micrograms/ |
| Substance | mL) |
| Activin A (human, recombinant) | 0.0005 |
| CGRP alpha, (Calcitonin Gene Related Peptide, rat) | 0.1905 |
| C natriuretic peptide) (human, porcine, rat: frag 32-53) | 0.10985 |
| (CNP) | |
| Cholera Toxin B Subunit, recombinant | 0.0125 |
| DEXamethasone (9 alpha-fluoro-16alpha- | 0.002 |
| methylprednisolone)(hydrocortisone analogue) | |
| FGF acidic (aFGF = FGF1), Recombinant Human | 0.0025 |
| GLP-1 (7-36) amide, human (Glucagon-Like Peptide | 0.033 |
| 1) | |
| Glucose (base should be I0ow; 0.9 ug/ml) | 1.08 |
| Insulin, human (low [] in base media 1 ug/ml) | 9.5 |
| LIF, human (leukemia inhibitory factor, human) | 0.0025 |
| PDGF AA + PDGF BB MIX | 0.005 |
| TGF alpha | 0.001 |
| Prolactin (human, recombinant)(a plasma growth | 0.0012 |
| hormone) | |
| Trolox (soluable Vitamin E) (C14H18O4) | 0.625 |
| GRP (Gastrin Releasing Peptide, Human) | 0.143 |
| IGF-1, recombinant human | 0.0025 |
| IGF-2, recombinant human | 0.0025 |
| Laminin | 2.25 |
| Met-Enkephalin (tyr-gly-glyl-phe-met) | 0.003 |
| Sonic Hedgehog (mouse, recombinant) | 0.025 |
| Substance P (full length) (H1875 is frag 1-4) | 5 |
| FGFb (=FGF2), human Recombinant | 0.0025 |
| VEGF | 0.0025 |

In a preferred embodiment of this aspect of the invention, the culture medium comprises at least one (or as many as all 10) of the following differentiation promoting DPFs:.C-Natriuretic Peptide (CNP), Calcitonin Gene Related Peptide, Cholera Toxin B Subunit, Dexamethasone, Gastrin-Releasing Peptide, Laminin, Met-Enkephalin, PDGFAA+PDGFBB, Sonic Hedgehog, and Substance P.

In a preferred embodiment, the culture medium that promotes the transformation of glandular epithelial cells into insulin producing cells consists of a 1:1 mixture of DMEM and Hams F12 plus the components listed in Table 2. This medium is sometimes referred to herein as “Media or Medium G9.”

TABLE 2

| | | | Concentration |
|---|---|---|---|
| | | | (Final) |
| | Factor | Substance | ug/ml |
| 1 | ANP | Atrial Natriuretic | 0.1530 |
| | | Peptide, Rat (28 amino | |
| | | acids) | |
| 2 | CCK-8-frag-amide | CCK8, Fragment 26-33 | 0.0250 |
| | | Amide | |
| | | (Cholecystokinin) | |
| | | (Asp-Tyr(SO3H)-Met- | |
| | | Gly-Trp-Met-Asp-Phe- | |
| | | NH2) | |
| 3 | Caerulin sulfate | Caerulein (Pyr-Gln- | 0.0300 |
| | | Asp-Tyr(SO3H)-Thr- | |
| | | Gly-Trp-Met-Asp-Phe- | |
| | | NH2 | |
| 4 | Cholera Toxin-B | Cholera Toxin B | 0.0125 |
| | | Subunit, recombinant | |
| 5 | Dex | DEXamethasone (9 | 0.0020 |
| | | alpha-fluoro-16alpha- | |
| | | methylprednisolone) | |
| | | (hydrocortisone | |
| | | analogue) | |
| 6 | FGF-7 | FGF7 (KGF) | 0.0025 |
| 7 | GLP-1 | GLP-1 (7-36) amide, | 0.0330 |
| | | human (Glucagon-Like | |
| | | Peptide 1) | |
| 8 | GRP | GRP (Gastrin | 0.1430 |
| | | Releasing Peptide, | |
| | | Human) | |
| 9 | Gastrin-1 | Gastrin I Human | 0.0000 |
| 10 | Glucose | Glucose (base should | 1.0800 |
| | | be I0ow; 0.9 ug/ml) | |
| 11 | HGF | Hepatocyte Growth | 0.0025 |
| | | Factor (HGF) | |
| | | recombinant | |
| 12 | IGF-1 | IGF-1, recombinant | 0.0025 |
| | | human | |
| 13 | IGF-2 | IGF-2, recombinant | 0.0025 |
| | | human | |
| 14 | Insulin | Insulin (low [] in base | 9.5000 |
| | | media 1 ug/ml) | |
| 15 | Leu-Enkephalin | Leu-Enkepthalin (tyr- | 0.0030 |
| | | gly-gly-phe-leu) | |
| 16 | Nicotinamide | Nicotinamide | 610.0000 |
| 17 | PTHRP-frag-1-34 | pT II RP (Parathyroid | 0.2060 |
| | | Hormone Related | |
| | | Peptide (1-34), human) | |
| 18 | Progesterone | Progesterone | 0.0030 |
| 19 | Prolactin | Prolactin (human, | 0.0012 |
| | | recombinant)(a plasma | |
| | | growth hormone) | |
| 20 | Retinol acetate | Retinoic Acid (Vitamin | 0.0250 |
| | | A) | |
| 21 | SHH | Sonic Hedgehog | 0.0250 |
| | | (mouse, recombinant) | |
| 22 | Sodium selenite | Selenium (Selenous | 0.0250 |
| | | Acid, Na salt) | |
| 23 | Soybean trypsin inhibitor | Trypsin Inhibitor, | 0.5000 |
| | | soybean (type I-S) | |
| 24 | TGF-beta sRII | TGF beta sRII (soluable | 0.0050 |
| | | receptor type 2) | |
| 25 | Transferrin | transferrin | 2.7500 |
| 26 | Trolox | Trolox (soluable | 0.6250 |
| | | Vitamin E) (C14H18O4) | |
| 27 | VIP | Vasoactive Intestinal | 0.0665 |
| | | Peptide (VIP), human | |
| 28 | aFGF | FGF acidic | 0.0025 |
| | | (aFGF = FGF1), | |
| | | Recombinant | |
| | | Human | |
| 29 | bFGF | FGFb (=FGF2), human | 0.0025 |
| | | Recombinant | |
| 30 | n-Butyric acid | n Butyric Acid, Sodium | 4.5400 |
| | | Salt | |

The components of this aspect of the invention may also be conveniently packaged in the form of a kit. The kit may include, for example, 1) a cell culture medium such as DMEM, Hams F12, or a combination thereof; 2) a serum-free medium supplement containing: BSA and the DPFs Activin A, acidic FGF, basic FGF, C-Natriuretic Peptide (CNP), Calcitonin Gene Related Peptide, Cholera Toxin B Subunit, Dexamethasone, Gastrin-Releasing Peptide, Glucagon-like Peptide-1 (GLP-1), Glucose, IGF1, IGF2, Insulin, Laminin, LIF, Met-Enkephalin, PDGFAA+PDGFBB, Prolactin, Sonic Hedgehog, Substance P, TGF-alpha, Trolox (alpha-tocopherol derivative), or VEGF, or two or more of these components in combination, in suitable amounts to yield the concentrations noted in Table 1 in the completed medium; and 3) tissue culture dish(es) with at least one collagen-1 coated tissue culture surface (or collagen-1 coated inserts for use in culture dishes or other laboratory ware). The kit may also optionally include a tissue culture dish and/or other cell culture accessories and additional reagents that may be required to carry out epithelial cell culture and differentiation. In other embodiments, the kit may contain any of the media or media components discussed herein.

Culture systems consisting of scaffolds, collagen coated flasks or other vessels and serum-free base medium may be packaged along with the DPF(s) as a separate vial that would be added to the culture medium just prior to use. The DPF combination can be added to a variety of base media to accomplish the same end, e.g., growth and differentiation of primary pancreatic acinar cells in vitro. Such culture systems may also be useful for other cell types, particularly other epithelial cells derived from glandular tissues, including those from liver, pancreas, intestine, prostate, and breast.

The invention also provides a method for converting glandular epithelial cells into insulin-producing cells comprising culturing the glandular epithelial cells in the cell culture system described above. The method may further comprise removing the culture medium from the cell culture, re-feeding the cell culture with a serum-free medium with glucose, and measuring proinsulin production C-peptide production, or insulin release.

Furthermore, the invention provides an isolated population of insulin-producing cells containing cytoplasmic granules with immunodetectable proinsulin, insulin, and/or c-peptide that is derived from a population of cells of which a subset of cells expressed at least one marker associated with IP cells (e.g., expressed some acinar-associated genes, as well as some liver-associated genes, including, e.g., ductular cytokeratins (CK7, CK8, CK18 and CK19), HNF1, alpha-1 antitrypsin, pi-glutathione s transferase (pi-GST), liver-specific bHLH transcription factor, Thy-1, C/EBP-alpha and C/EBP-beta, and expressed little if any of the pancreas-associated genes carbonic anhydrase, cystic fibrosis transmembrane conductance regulator (CFTR), elastase and amylase).

By an “isolated” cell or population of cells is meant herein that the cell or cell population is removed from its original environment (e.g., the natural environment if it is naturally occurring), and isolated or separated from at least one other component with which it is naturally associated. For example, a naturally-occurring cell present in its natural living host is not isolated, but the same cell, separated from some or all of the coexisting materials in the natural system, is isolated. Such cell or cell populations could be part of a cell culture or cell population, and still be isolated in that such culture or population is not part of its natural environment.

In one preferred embodiment, the insulin-producing cells are derived from glandular epithelial cells obtained from mammalian pancreas, such as primary acinar cells.

The data disclosed in the examples below are generated from freshly isolated human pancreatic cells. The expansion of primary human pancreatic cells in these conditions produces cultures with a mixed epithelial IP phenotype, suitable for in vitro studies of IP cells for a variety of purposes, and suitable for transplantation in vivo for cell therapy for the treatment of diseases such as diabetes. The IP cells generated by these methods may also be useful in the study of pancreatic cell biology, as normal controls in the study of pancreatic epithelial cancers, and to test the effects of drugs/compounds on normal pancreatic epithelial cells (ductal or acinar). Furthermore, the cells may be further cultured to yield insulin-producing cells as demonstrated below.

### BRIEF DESCRIPTION OF THE DRAWINGS

FIGS. 1A-D show microscopic images after treatment of starting material with antibodies to amylase (FIG. 1A), insulin (FIG. 1B), and CK19 (FIG. 1C) and the composition of the cell pellet of freshly isolated primary human pancreatic cells (FIG. 1D).

FIG. 2 shows growth curves constructed from primary human pancreatic cultures grown in commercial medium (with serum) or in the described pancreatic cell medium (PCM) with serum.

FIG. 3 shows a comparison of cell expansion in the base medium composition described vs. base medium+soluble growth factors (serum-free formula) vs. base medium+fetal bovine serum.

FIGS. 4A-B shows the effect of different culture surfaces on total cell number (FIG. 4A) and cell phenotype (FIG. 4B) after expansion.

FIGS. 5A-B show a comparison of cell phenotype after expansion in serum-containing (5A) and serum-free (5B) medium containing all soluble active factors.

FIG. 6 shows high power images of cell cultures expanded in various conditions, including serum-free base media supplemented with 3 soluble active factors, HGF, EGF & TGFA. Note epithelial morphology.

FIG. 7 shows a demonstration of growth of IP cells on ECM-coated surfaces as determined by metabolic activity assay over time. Note superior growth when Collagen I surface is combined with the media formulation described herein, yielding results superior to the combination of Matrigel and commercial media with serum.

FIG. 8A (upper left) shows expression of amylase by acinar cells after two days of culture (red staining), FIG. 8B (lower left) shows expression of CK19 (green staining), FIG. 8C (right) shows an overlay of the two images, showing co-expression (yellow) in a large proportion of cells.

FIG. 9 shows changing phenotype of primary acinar cells in culture over 5 days. Amylase is red, CK19 is green. Note appearance of yellow (amylase+CK19) on Day 2 and 3.

FIGS. 10A and 10B show primary human pancreatic cells that were expanded in serum-containing medium on Collagen I coated surface. Images were analyzed to determine total cells (FIG. 10A, blue nuclei) and total positive cells (FIG. 10B, blue nuclei surrounded by green staining for CK19).

FIG. 11 shows light microscopic (200×) appearance of pancreatic acinar cells cultured on a collagen I surface with all DPFs (Activin A, 0.5 ng/ml; acidic FGF, 2.5 ng/ml; basic FGF, C-Natriuretic Peptide (CNP), 0.11 μg/ml; Calcitonin Gene Related Peptide, 0.19 μg/ml; Cholera Toxin B Subunit, 12.5 ng/ml; Dexamethasone, 0.002 μg/ml; Gastrin-Releasing Peptide, 0.143 μg/ml; Glucagon-like Peptide-1 (GLP-1), 0.033 μg/ml; Glucose, 1.08 μg/ml; IGF1, 0.0025 μg/ml; IGF2, 0.0025 μg/ml; Insulin, 9.5 μg/ml; Laminin, 2.25 μg/ml; LIF, 0.0025 μg/ml; Met-Enkephalin, 0.0030 μg/ml; PDGFAA+PDGFBB (0.0050 μg/ml: 0.0025 μg/ml of PDGFAA+0.0025 μg/ml PDGFBB); Prolactin, 0.0012 μg/ml; Sonic Hedgehog, 0.025 μg/ml; Substance P, 5.0 μg/ml; TGF-alpha, 0.0010 μg/ml; Trolox (alpha-tocopherol derivative), 0.625 μg/ml; and VEGF, 0.0025 μg/ml).

FIG. 12A (top right panel) shows immunocytochemical analysis with CK19 antibodies (green).

FIG. 12B (lower right panel) shows immunocytochemical analysis with C-peptide antibodies (red).

FIG. 12C (left panel) shows an overlay image demonstrating the colocalization of CK19 and C-peptide (orange). Blue portions are DAPI stained nuclei.

FIG. 13A shows insulin release upon glucose challenge in IP cells that have not been detached and relocated (subcultured) during the growth and differentiation process.

FIG. 13B shows insulin release upon glucose challenge in IP cells that have been subcultured according to Example 10.

FIG. 13C shows C-peptide release upon glucose challenge in IP cells that have not been subcultured according to Example 10.

FIG. 14 shows the Insulin/DNA ratio in subcultured and nonsubcultured cells that are treated with Combinations 1, 2 and 3 of DFP media, as described in Example 11.

FIG. 15 shows insulin release in response to base level glucose (5 mm) and a glucose challenge (22 mm) over 10 days of culture in PCM and DPF media, as described in Example 13.

FIG. 15A shows insulin release in response to base level glucose (5 mm) and a glucose challenge (22 mm) over 14 days of culture in PCM and DMG9 media, as detailed in Example 14.

FIG. 16 is a graphical representation of the characteristics of the 17 classes of genes shown in Table 6, as indicated in the last column of the Table, as detailed in Example 14.

### DETAILED DESCRIPTION OF THE INVENTION

In describing preferred embodiments of the present invention, specific terminology is employed for the sake of clarity. However, the invention is not intended to be limited to the specific terminology so selected. It is to be understood that each specific element includes all technical equivalents, which operate in a similar manner to accomplish a similar purpose. Each reference cited here is incorporated by reference as if each were individually incorporated by reference.

The following abbreviations are used:

BSA: bovine serum albumin

BMP Bone Morphogenetic Protein

bHLH: basic helix loop helix

DMEM: Dulbecco's Modified Eagle's Medium

TGFβ1: Transforming Growth Factor β1

ECM: extracellular matrix molecules; naturally occurring proteins produced by cells of a tissue that provide structural support as well as a source of cellular signals related to adhesion. Examples are collagen, vitronectin, fibronectin, laminin.

EGF: Epidermal Growth Factor

Hams F12: Ham's Nutrient Mixture F12

HGF: Hepatocyte growth factor

HNF-1: Hepatic nuclear factor 1

IGF1: Insulin-like growth factor 1

IGF-II: Insulin-like growth factor 2

IP cells: Intermediate progenitor cells derived from an epithelial cell, such as, e.g., a pancreatic acinar cell or a liver cell, wherein the derived cells express some acinar-associated genes, as well as some liver-associated genes, including, e.g., cytokeratins (CK7, CK8, CK18 and CK19), HNF1, alpha-1 antitrypsin, pi-glutathione s transferase (pi-GST), liver-specific bHLH transcription factor, Thy-1, C/EBP-alpha and C/EBP-beta, and express little if any of the pancreas-associated genes carbonic anhydrase, cystic fibrosis transmembrane conductance regulator (CFTR), elastase and amylase).

PDGF-A: Platelet derived growth factor alpha

PDGF-B: Platelet derived growth factor beta

TGFA, TGF-α: Transforming Growth Factor α

As used herein, the term “culture system” is intended to mean a system for growing and/or differentiating cells in culture, which comprises a cell attachment surface, preferably one that also stimulates cellular expansion, and a culture medium, which includes effective amounts of one or more factors, or serum (e.g. fetal bovine serum), added to a base medium composition.

When referring to active soluble factors and DPFs herein, “effective amount” means an amount that either alone or in combination with other included factors is effective in promoting either expansion and differentiation into IP cells, or into insulin-producing cells, as applicable.

### EXAMPLES

### I. Expansion and Transdifferentiation of Primary Acinar Cells into Glandular Epithelial Cells (Culture Phase I)

Materials and Methods:

Starting Material: Primary human pancreatic acinar cells are collected as waste from standard COBE gradient preparation of islet cells for transplantation (Lake et al., 1989). After density gradient centrifugation, the islets are present as a layer between 1.063 density and 1.10 density, and the remaining cells are collected as the pellet that sediments to the bottom of the gradient based on density. Approximately 48 hours after collection of the cells at the transplant center are received by the inventors in non-tissue-culture treated polystyrene flasks and are suspended in RPMI+10% fetal calf serum at a density of approximately 2.0 million cells/ml. Cell number and viability is assessed by trypan blue exclusion and enumeration on a hemacytometer by light microscopic observation.

Phenotypic Evaluation of Starting Material. A preparation of starting material was formalin fixed and paraffin-embedded as a cell pellet about 24 hours after initial harvest of the pancreatic cells. Paraffin sections were prepared, placed on slides, and subjected to immunocytochemical analysis with antibodies to insulin (Biogenex, San Ramon, Calif.), CK19 (Biogenex), and Amylase (Biogenex). A minimum of (3) sections per sample were assessed with each marker. All antibody staining was carried out according to the manufacturer's suggestion with pre-diluted commercial antibodies. For CK19, a 3 minute treatment with pepsin enzyme (Biogenex) preceded the blocking step for the purpose of antigen retrieval. Briefly, the sections were rehydrated through graded ethanols, followed by a 15 minute incubation in phosphate buffered saline (PBS) without calcium and magnesium. Protein Blocker (Biogenex) was added for 30 minutes prior to addition of primary antibodies. After (3) 5-minute washes, biotinylated secondary antibody (Biogenex) was added at a 1:100 dilution and sections were incubated for 30 minutes at room temperature. After (3) 5-minute washes, Alexa488 or Alexa-596-conjugated StreptAvidin (Molecular Probes, Eugene, Oreg.) was added for fluorescent visualization. For each slide, a minimum of (3) 200× images were captured using a Nikon fluorescent microscope fitted with a SPOT camera (Diagnostic Systems, Inc., Webster, Tex.). The images were assessed quantitatively using image analysis software (MetaMorph/Universal Imaging Corporation, Downington, Pa.) to determine relative fraction of insulin-positive, CK19+, and amylase+ cells. Insulin+ cells are the beta cells of the islets, CK19+ cells are the primary ductal cells, and amylase+ cells are the acinar cells (see Example 1).

### Example 1

### Characterization of Cell Culture Conditions

A. Serum-Free Medium

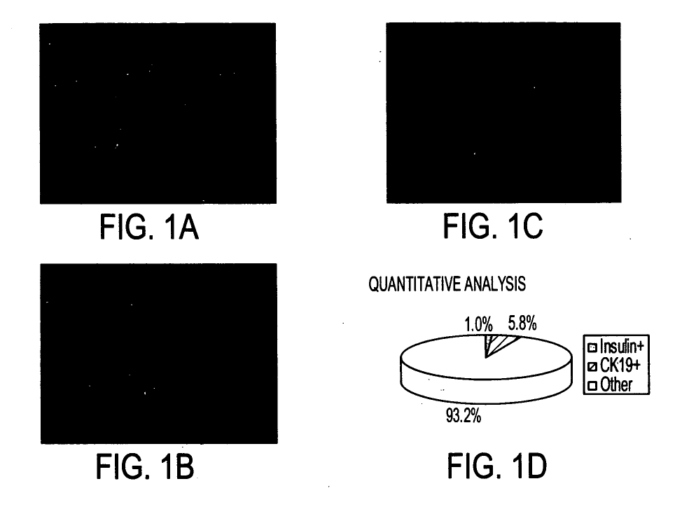

Freshly isolated primary human pancreatic cells were collected as a pellet from a COBE cell separator, fixed in formalin, paraffin-embedded, sectioned, and analyzed with antibodies to amylase, CK19, and Insulin. Images (FIGS. 1A and 1B) were collected on a Universal Imaging System (Universal Imaging Corporation) and analyzed with MetaMorph Software. This cell pellet (FIG. 1C) was comprised of 1.0% insulin+ cells (beta cells of the islet), 5.8% CK19+ cells (primary ductal cells), and 93.2% amylase+ and unlabeled (acinar cells and other cell types).

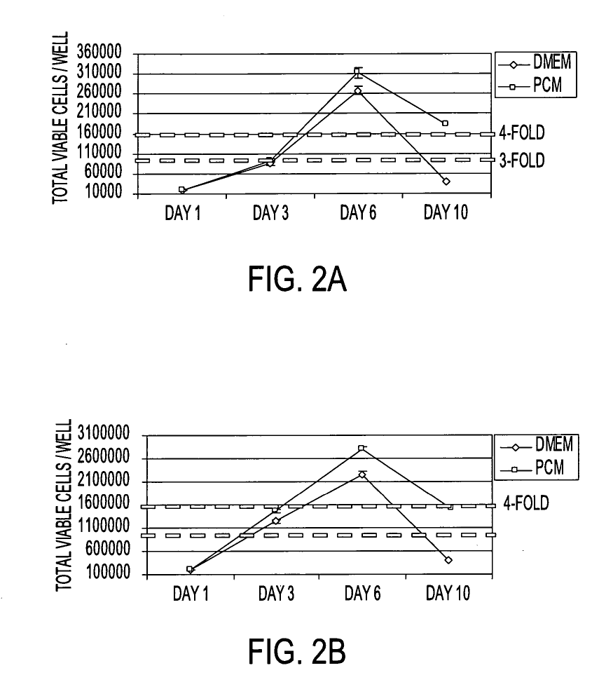

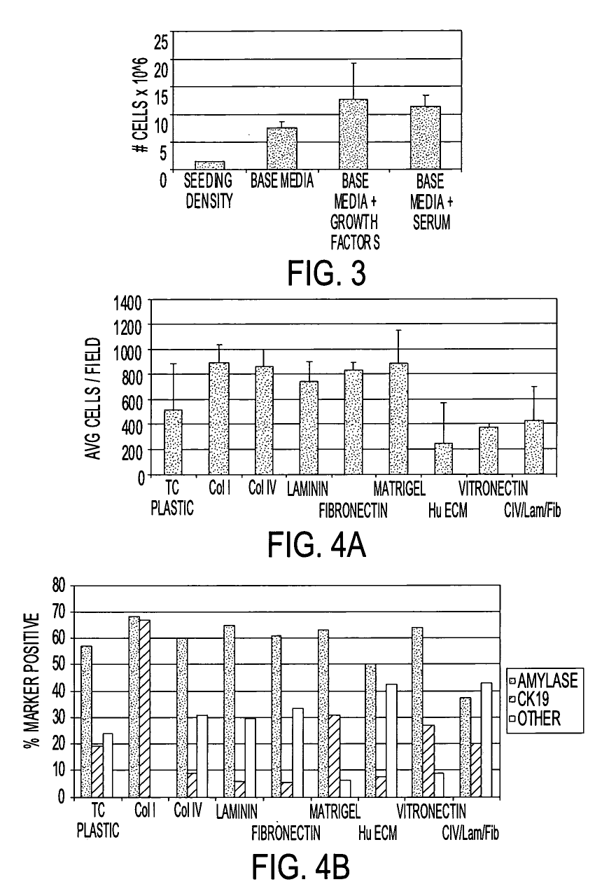

Primary human pancreatic cells were then seeded at 10⁴ or 10⁵ cells/ cm² onto tissue culture treated polystyrene in either DMEM commercial medium plus 10% fetal bovine serum or in PCM plus 10% fetal bovine serum. Replicate cultures were harvested at 3 day intervals via trypsinization and live cells (as determined by trypan blue exclusion) and enumerated on a hemocytometer. The results (shown in FIG. 2) demonstrate that the (serum-containing) medium formulation described herein is superior to commercial media formulation for growth and maintenance of primary pancreatic cells. FIG. 3 compares the results of expanding the cells for 6 days in base medium, base medium plus all of the soluble active factors [HGF, ˜1-˜20 ng/ml, preferably ˜5.0 ng/ml; TGFA, ˜1-˜10 ng/ ml, preferably ˜2 ng/ml; Betacellulin, ˜0.5-˜20 ng/ml, preferably ˜10 ng/ml; Gastrin 1, ˜0.05-˜10 ng/ml, preferably ˜0.06 ng/ml; Prolactin, ˜1.0-˜10 ng/ml, preferably ˜2.4 ng/ml; and IGF1, ˜5-˜100 ng/ml, preferably ˜5 ng/ml] and base medium plus 10% serum. The serum-free media formulation meets/exceeds expansion provided by media+serum.

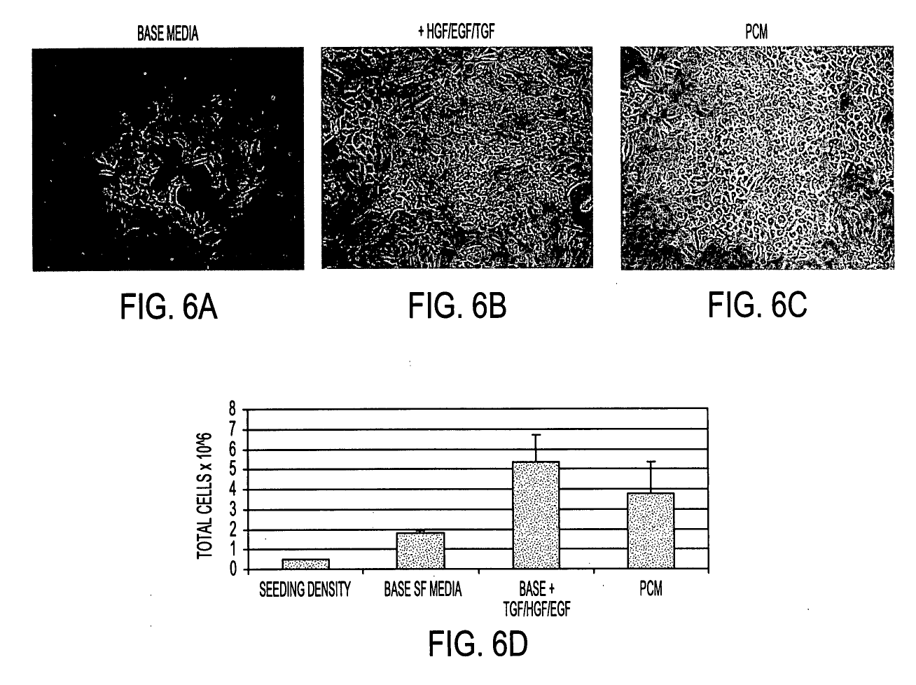

The cell expansion experiment was repeated essentially as above, except that the base medium was supplemented with only three of the soluble active factors: TGF, HGF, and EGF. FIG. 6D compares the results of expanding the cells in the various media; FIGS. 6A, 6B and 6C show high power images of the cell cultures expanded under the various media conditions.

B. ECM Surfaces

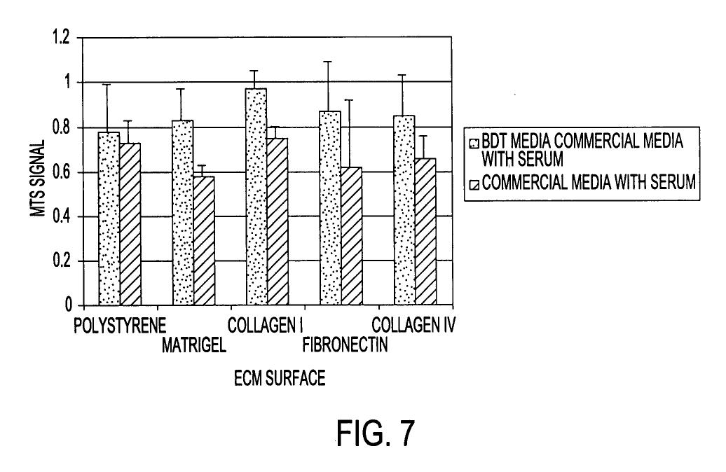

The attachment of primary human pancreatic cells was evaluated by counting the number of attached cells vs. the number of cells initially seeded on a panel of ECM surfaces comprised of Collagen I (1 μg/cm²), Fibronectin (3 μg/cm²), Laminin (2 μg/cm²), Vitronectin (1 μg/cm²), Matrigel (1 μg/cm²), Human ECM (1 μg/cm²), or Poly-D-Lysine (3 μg/cm²). In one condition, a mixture of Collagen IV, Laminin, and Fibronectin was utilized. ECMs were placed into solution at the above concentrations and allowed to coat tissue culture-treated polystyrene surfaces according to manufacturer's suggestions of 1 hour at room temp. Excess ECM solution was then removed and surfaces were rinsed twice in water. Just before seeding cells, the water was aspirated, then cells were seeded onto the ECM surface at a density of 1×10⁵ cells/cm² in growth medium (PCM) composed of DMEM:HamsF12 mixture (1:1) with 4 mM glutamine, 1×ITS supplement (GIBCO 51500-056), 10% Fetal Calf Serum (Inactivated, Qualified, GIBC 26140-079), and 10 ng/ml Epidermal Growth Factor (EGF) (BD 4001). Cells were seeded onto tissue-culture polystyrene surface as a control. After 18 hours, unattached cells were washed away and remaining attached cells were re-fed with PCM and allowed to grow for 7 days prior to evaluation. Cultures were fixed in 10% formalin and subjected to immunocytochemistry with antibodies for CK19 and Amylase as described previously to determine phenotypic composition. Cells were counterstained with DAPI fluorescent blue nuclear stain to visualize individual cell nuclei for cell counting. The metabolic activity of cells subjected to the various conditions was determined by an MTS assay. Viable cells were measured using the MTS assay (Promega CellTiter 96 Aqueous One Solution Cell Proliferation Assay), a calorimetric method for determining the number of viable cells in proliferation or cytotoxicity. The results of this analysis are shown in FIG. 7.

### Example 2

### Further Studies with ECM Surfaces and Various Media Components

Primary pancreatic cells, composed of >90% non-islet/non-duct cells, were plated onto various coated surfaces at a density of 28,900 cells/well (10⁵ cells/cm²). Unattached cells were washed off after 18 hours, and cultures were re-fed and allowed to grow for 8 days. Cultures were fixed in formalin (10%) and subjected to phenotypic analysis with antibodies to CK19 and Amylase. The results are shown in FIGS. 4A-B. While Collagens I, IV, Laminin, Fibronectin, and Matrigel provide a suitable surface for cell attachment and expansion, maintenance of acinar (amylase+) phenotype along with the presence of an increased proportion of cells with a glandular epithelial phenotype (CK19+) was superior on Collagen I. More than 50% of cells analyzed expressed amylase and more than 50% of cells analyzed expressed CK19, suggesting that a subpopulation of cells in these experimental conditions express both markers.

Tissue culture-treated polystyrene culture surfaces were coated with Collagen I as described above. Tissue culture medium (PCM) was prepared as described above. In some cases, serum was replaced with Fraction V BSA (99% pure, heat inactivated, Sigma), along with combinations of soluble growth factors, including IGF1, IGF2, betacellulin, HGF, EGF, and TGF-alpha. Optimal seeding density is between 10⁴ and 10⁵ cells/cm², as demonstrated in Example 3. Cells were seeded onto collagen-coated flasks (150 cm² ) at 1.5×10⁶ cells/flask in PCM. After an ˜18 hr attachment period, unattached cells were washed away with gentle aspiration/rinse, followed by re-feeding with fresh medium. Cultures were monitored over time by metabolic assay (MTT) and by trypsinization and cell counting, to establish cell number (see example 3). Cell phenotype at the end of the culture period was assessed as follows: small-scale cultures were set up simultaneously in 96-well plates. At the end of the culture phase, monolayer cells were fixed in 10% formalin for a minimum of 1 hour. After formalin was removed and monolayers were rinsed, cultures were subjected to immunocytochemistry as described in previous section for CK19, amylase, insulin, and vimentin (a marker of fibroblasts). The relative fraction of CK19+ cells was determined by quantitative image analysis as described above (see Example 4). After formalin was removed and monolayers were rinsed, cultures were subjected to immunocytochemistry as described in previous section for CK19 and vimentin (a marker of fibroblasts). Cells were also stained with amylase antibodies, but did not produce positive results due to release of digestive enzymes, such as amylase, by the cells over time in culture. The relative fraction of CK19+ cells was determined by quantitative image analysis as described above (see example 4). Acquisition of ductal markers by acinar cells was verified by demonstrating concomitant expression of CK19 and amylase in cell subpopulations during days 2-3 of culture (see example 5). For these experiments, CK19 primary antibodies were reacted with formalin-fixed cell cultures, followed by visualization with Alexa488-conjugated Goat anti-mouse IgG (Molecular Probes). Then, cells were subjected to a blocking step (Protein Blocker, BioGenex), followed by application of the second primary antibody (anti-amylase). Visualization of the amylase was accomplished by application of Alexa594-conjugated Goat Anti-Mouse IgG. Images were collected as described above. At the end of a 7-day culture period in the conditions described herein, between 65-90% of the cells in the culture express CK19, while less than 20% express vimentin (see example 6). Variations in the relative proportion of CK19+ cells probably reflect heterogeneity due to age, gender, and other unique characteristics of individual patients.

### Example 3

### Density of Cell Seeding

Primary pancreatic cells were seeded at (3) densities on tissue-culture treated polystyrene dishes (60 mm) and fed with PCM. Light microcopic observations were made daily. At the 24-hour timepoint, dishes were sacrificed and stained with trypan blue to assess viability. The results are shown in Table 3.

TABLE 3

| Seeding | | | After 3 days |
|---|---|---|---|
| Density | At 24 Hours: | At 48 Hours: | Growth: |
| 10⁴ cells/cm² | Most cells attached, | Mitotic Figures present | Epithelial |
| | trypan blue negative | (light microscopy) | monolayer |
| | (live) | | forming |
| 10⁵ cells/cm² | Most cells attached, | Mitotic Figures present | Epithelial |
| | trypan blue negative | (light microscopy) | monolayer |
| | (live) | | forming |
| 10⁶ cells/cm² | Some cells attached, | Few Mitotic Figures | Cells are |
| | most are trypan blue | present (light | detached; |
| | positive (dead) | microscopy) | some |
| | | | fibroblasts |
| | | | present |

### Example 4

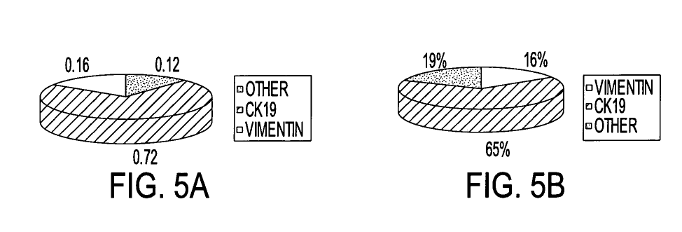

Cells were grown on a Collagen I surface, at 37° C. in 21% oxygen, in PCM medium or in base medium with 2 % BSA, 2 ng/ml TGF-α, 10 ng/ml EGF, and 10 ng/ml HGF. After 7 days, cultures were fixed in 10% formalin and subjected to immunocytochemical analysis with fluorescent detection, followed by automated image collection and analysis. The results are shown in FIGS. 5A and 5B. Fibroblast (vimentin+) fraction, glandular epithelial cell fraction (CK19+), and fraction of unlabeled cells (Other) are similar after expansion. This suggests that replacement of serum with the serum-free medium maintains fraction of CK19+ cells without overgrowth of fibroblasts as compared to cells grown in serum-containing media.

### Example 5

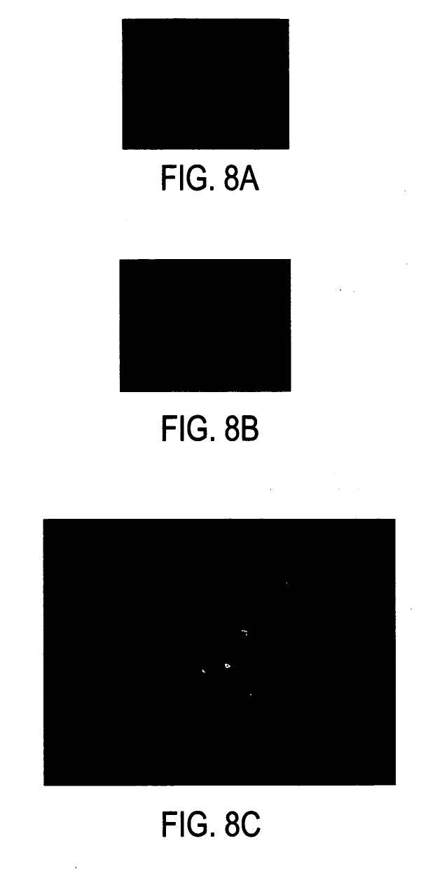

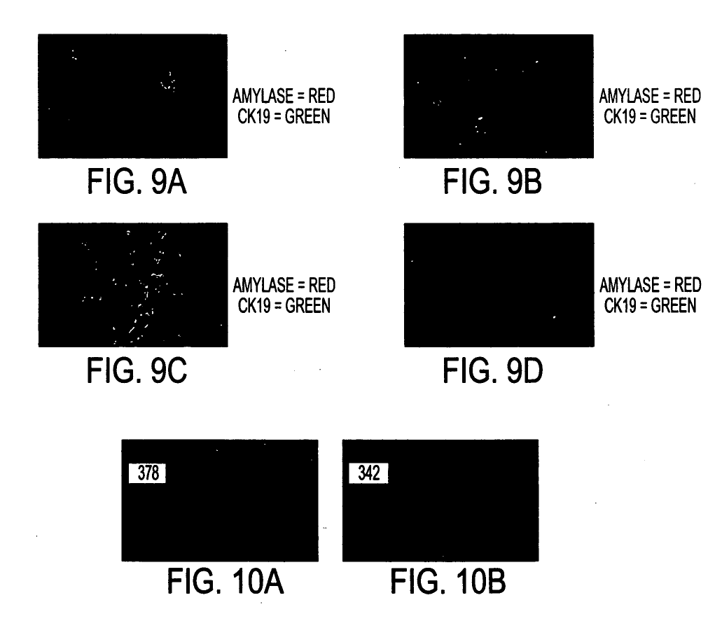

Primary pancreatic acinar cells were cultured for several days in a 1:1 ratio of DMEM and HamsF12, with 10% fetal bovine serum, 0.01 mg/ml insulin, 0.0055 mg/ml transferrin, 0.0067 μg/ml sodium selenite, 10 ng/ml EGF, 4 mmol/liter glutamine and antibiotics. After 2 days of culture (4 days ex vivo), expression of amylase by the acinar cells is still strong (FIG. 8A, upper left panel, red staining) as determined by immunocytochemistry. Expression of CK19 is also apparent (FIG. 8B, lower left panel, green staining). Overlay of the two images (FIG. 8C) demonstrates clear co-expression of amylase and CK19 in a large proportion of the cells, indicating that an intermediate cell exists from active conversion of amylase+ acinar cells to an amylase+/CK19+ mixed acinar/ductal phenotype (AD cells). Daily evaluation of cultures (FIG. 9) demonstrated that onset of CK19 expression begins around Day 2 of culture and by Day 5 cultures have lost most immunodetectable amylase expression and CK19 expression is prevalent.

### Example 6

After 7 days of growth in PCM/Collagen I surface, cells were fixed, stained with antibodies to CK19, and counterstained with nuclear DAPI. Total cell number was evaluated by automated image analysis (FIG, 10A left panel, blue-stained cell nuclei), while CK19+ cells were counted (FIG. 10B, right panel, green-stained cell cytoplasm). Of 378 total cells, 342 were immunopositive for CK19 (90%). After approximately 7 days of culture using conditions described herein, the acinar cells have concrete ductular characteristics, now referred to as IP cells. For most primary human cultures, more than 80% of cells in the culture after about 7 days express markers such as CK19 that are associated with ductular cells from a variety of tissues.

### Example 7

### Gene Expression Analysis of 7-Day Cultures (IP Cells)

Two independent IP cell cultures were subjected to Clontech 8K Atlas Gene Array analysis. IP cells were obtained by culturing primary acinar cells in a cell culture system comprising PCM and a Collagen I surface. Monolayer cultures were rinsed 2× with PBS, then detached from the flasks with 0.25% trypsin. Cells were pelleted by centrifugation at 1,200 RPM for 3 minutes in a swinging bucket centrifuge. Cell pellets were resuspended and washed 2× in PBS before a final centrifugation at 1,200 RPM for 3 minutes as described above. The supernatant was discarded and gently aspirated to remove as much liquid as possible from the cell pellet, which was then quick-frozen in a dry-ice/ethanol bath and stored at −80° C. until transfer to BD Clontech where gene expression analysis was performed, using conventional techniques.

Labelled P-33 cDNA probes were prepared from the 30 μg of total RNA from each sample by first enriching for poly A+RNA using a streptavidin-magnetic bead separation method that is part of the Atlas Pure Total RNA Labeling system. The labeled probes from each sample were hybridized with the plastic human 8 K gene arrays for about 16 hours, the arrays were washed and imaged according to the Atlas array protocols. The Atlas image 2.7 software was used to align array images with the array grid template and to exclude false background signals or false signals due to strong signal bleedover. The transcript signals were then extracted from these aligned arrays using the Atlas Image 2.7 software and further statistical analysis of the changes in gene expression were performed.

In general, mRNA transcription was assayed, by hybridization to suitable oligonucleotide probes. In a few cases, e.g., for CK19 and amylase, the protein expression product was measured, using conventional methods of immunohistochemistry. A summary of the expression by these cell populations of a selected set of genes is presented in Table 4. Table 4 contains a list of genes expressed in IP cells and a comparison of expression patterns in primary acinar cells and primary ductal cells. Gene products identified as “+” were expressed; those identified as “++” were strongly expressed. Gene products designated ® are found in regenerating pancreas.

TABLE 4

| | | | Primary |
|---|---|---|---|
| | | | Acinar |
| Gene | IP Cells | Primary Ductal Cells | Cells |
| Aquaporin 1 | + | ++(mRNA)/+(protein) | 0 |
| Aquaporin 5 | ++ | +(mRNA)/−(protein) | |
| Aquaporin 8 | + | − | + |
| Insulin Receptor Substrate-2 | + | ++ ® | |
| Protein Kinase B (AKT) | 0 | + | + |
| Calpain (mu) | ++ | − | ++ |
| CFTR (Cystic Fibrosis | 0 | 0 | 0 |
| Transmembrane | | | |
| Conductance Regulator) | | | |
| Claudin 2 | nd | ++ | − |
| Claudin 3 | + | + | + |
| Claudin 4 | + | + | + |
| Claudin 5 | nd | − | ++ |
| Carbonic Anhydrase II | 0 | 0 | 0 |
| Inositol 1,4,5 triphosphate | ++ | − | ++ |
| Rc, Type 3 | | | |
| MUC-1 | + | ++ | + |
| MUC-6 | ++ | + | |
| Cytokeratin 7 | ++ | ++ | − |
| Cytokeratin 8 | ++ | + | |
| Cytokeratin 18 | + | + | |
| Cytokeratin 19 | + | ++ | − |
| alpha v integrin | 0 | + | |
| alpha 3 integrin | ++ | n/a | |
| beta 3 integrin | 0 | + | |
| beta 4 integrin | + | n/a | |
| beta 5 integrin | 0 | + | |
| fibronectin | 0 | + | |
| collagen IV | + | + | |
| vitronectin | 0 | + ® | |
| MMP2 | 0 | Trace | |
| MMP9 | + | Trace | |
| TIMP1 | ++ | Trace | |
| TGF-alpha | Trace | ++ ® | |
| Gastrin | 0 | ++ ® | |
| ICAM-1 | 0 | 0 | |
| ICAM-2 | 0 | 0 | |
| ICAM-3 | 0 | 0 | |
| Pancreatitis Associated | ++ | + ® | Trace |
| Protein (PAP) | | | |
| Reg-1 | + | 0 | Trace |
| pyrimidinergic receptor P2Y | + | + | |
| Syndecan | + | +(low) | Trace |
| Glutathione S Transferase - | + | + | − |
| pi | | | |

### II. Transformation of Glandular Epithelial Cells into Insulin Producing Cells—Generating Insulin-Producing Cells by Differentiation of IP Cultures (Culture Phase II)

IP cultures can be utilized to generate insulin-producing cells by placing the cells in a second phase of culture that includes a surface, such as Collagen I, that promotes attachment of the IP cells combined with a defined medium formula that lacks serum but contains combinations of the following differentiation promoting factors: Activin A, acidic FGF, basic FGF, C-Natriuretic Peptide (CNP), Calcitonin Gene Related Peptide, Cholera Toxin B Subunit, Dexamethasone, Gastrin-Releasing Peptide, Glucagon-like Peptide-1 (GLP-1), Glucose, IGF1, IGF2, Insulin, Laminin, LIF, Met-Enkephalin, PDGFAA+PDGFBB, Prolactin, Sonic Hedgehog, Substance P, TGF-alpha, Trolox (alpha-tocopherol derivative), and VEGF. In the following examples, the base medium is composed of a 1:1 mixture of HamsF12 and DMEM with antibiotics and 0.2% Bovine Serum Albumin (Fraction V, heat inactivated 99% pure). In one example (Combination 1), the base medium contained Cholera Toxin B, Dexamethasone, GRP, GLP-1, Glucose, IGF-1, IGF-2, Insulin, Prolactin, Sonic Hedgehog, Trolox, aFGF, and bFGF. In another example (Combination 2), the base medium contained Activin A, CGRP-alpha, CNP, Glucose, GLP-1, IGF-2, Insulin, LIF, Met-Enkephalin, Prolactin, Sonic Hedgehog, aFGF, and vEGF. In a third example (Combination 3), the base medium contains Activin A, CGRP-alpha, Cholera Toxin B, Dexamethasone, Glucose, GLP-1, Insulin, LIF, Laminin, Met-Enkephalin, PDGFAA/BB, Sonic Hedgehog, Substance P, TGF-alpha, aFGF, and VEGF. The concentrations of these media supplements are listed in Table 1.

AD cells were placed into culture by either: 1) trypsinizing the cells from the surface on which they were generated, and redistribution onto a fresh attachment-promoting surface at a density of ˜5×10⁴ cells/cm², or 2) removing the medium, washing 2× in PBS to remove traces of old medium, and cultures re-fed with the new medium (described above) containing differentiation promoting factors. Cells are cultured for a period of 4-10 days at 37° C. and 21% oxygen. On Day 5, half of the medium is removed and replaced with an equal volume of fresh medium containing differentiation promoting factors.

Phenotypic Analysis of IP Cells After Differentiation Culture.

Morphological assessment of IP cells cultured in differentiation conditions described above was captured by light microscopy (see Example 8, below). The cellular phenotype of the cells comprising these cultures was assessed by immunocytochemistry as described above using monoclonal antibodies to vimentin, pro-insulin, C-peptide, MUC-1, and CK19 (See Example 10, below). Briefly, cultures were fixed with 10% formalin for 1 hour at room temperature, then washed with PBS and subjected to immunocytochemical protocol. (See Example 9, below).

Functional Analysis of IP Cells After Differentiation Culture.

The ability of the aggregated cell clusters to release insulin and C-peptide was assessed by subjecting the cultured cells to a glucose challenge as follows. Cells that had been cultured in differentiation medium for 7-10 days were washed 3× in PBS, then re-fed with either 1) base medium (described above) with 5 mM Glucose, or 2) base medium with 22 mM glucose. After 18 hours, the cell-conditioned medium was collected and subjected to ELISA analysis for insulin and C-peptide release (Diagnostic Systems Laboratories (DSL)). ELISAs were conducted using the standard range assay procedure according to manufacturer's specifications. Plates were incubated on a shaker during the assay and results were read in a Tecan spectrophotometric plate reader. Total ng of insulin or C-peptide per well were calculated for each media condition, for both 5 mM glucose media and 22 mM glucose media (See Example 10).

### Example 8

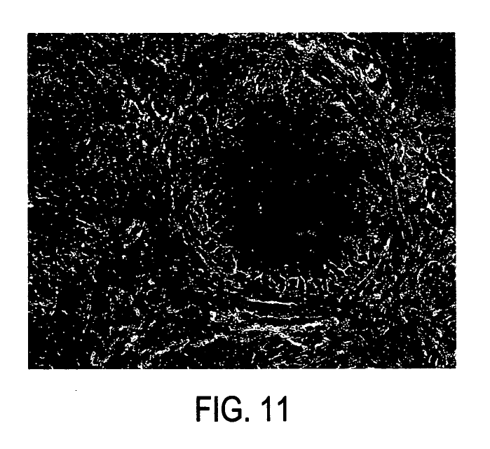

Pancreatic acinar cells were cultured in Base Medium+ITS+Serum (10%) for 1 week, then trypsinized (treated with 0.25% Trypsin without EDTA for 10 minutes at 37° C.) and transferred to a fresh collagen-1 coated surface and placed in a medium containing all 23 DFPs listed. Over a period of 3-5 days, the cells readily formed three-dimensional pod-like structures, clearly observable by light microscopy (FIG. 11). Some larger pods detached from the culture surface after about 4-6 days in culture, and remained viable, as determined by trypan blue exclusion. The pod-like structures were hypothesized to be aggregations of insulin-producing cells, and subjected to further analysis as described below.

### Example 9

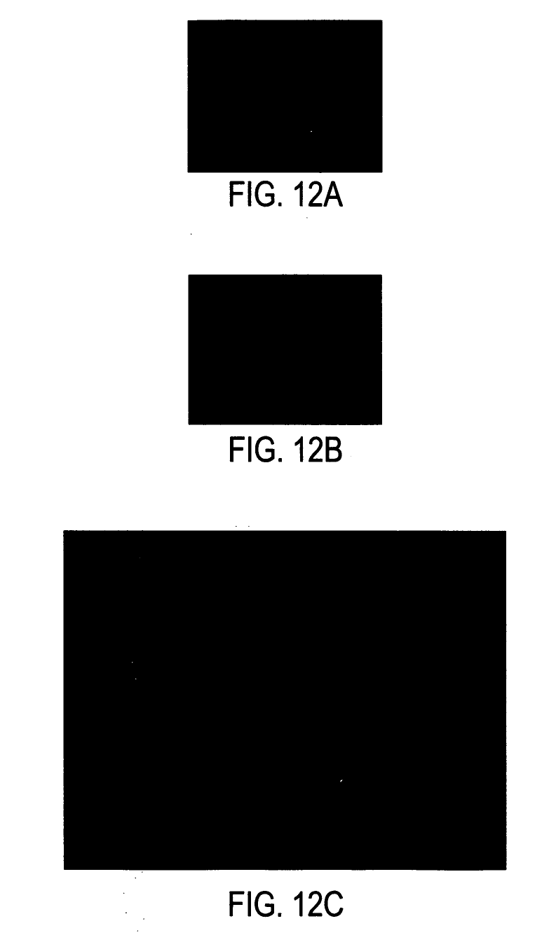

Pod-like structures, generated the same manner as described in the previous example, were fixed in 10% formalin and subjected to immunocytochemical analysis first with CK19 monoclonal antibodies, then with C-peptide monoclonal antibodies, as described above. FIG. 12A shows a group of cells (DAPI stained nuclei are blue), some of which are immunopositive for CK19 (green staining). FIG. 12B shows the same group of cells, many of which are positive for C-Peptide, which is produced when the proinsulin molecule synthesized within the cell is cleaved to yield mature insulin; the C-peptide stained cells are red, with a typical granular staining of the cytoplasm. FIG. 12C shows a higher power overlay image, demonstrating colocalization of CK19 and C-peptide in a small subset of cells. Co-stained cells appear yellow-orange on the overlay image.

### Example 10

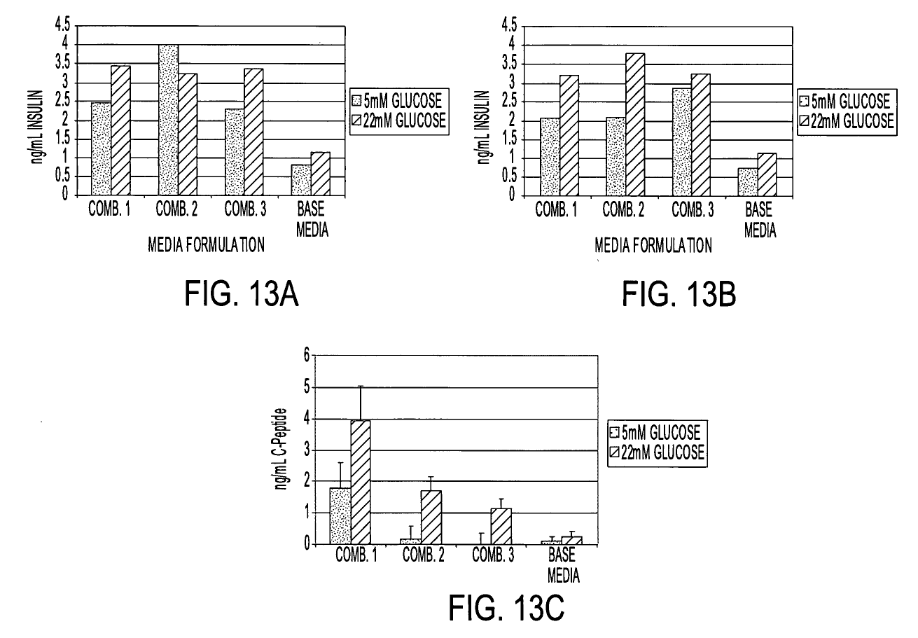

Cells cultured in base medium (negative control), or in Combinations 1, 2 and 3 of the differentiation promoting media, were evaluated for their ability to release insulin and C-peptide into the culture medium. In addition, we assessed whether increasing concentrations of glucose led to the release of a greater quantity of insulin and C-peptide, indicating an islet-like functionality. First, the cells were cultured for 1 week in base medium+EGF(10 ng/ml)+ITS+10% fetal bovine serum (PCM). Then, cells were either subjected to a wash and medium change (non-subcultured), or to a wash, trypsinization/detachment, reseeding, and medium change. Replicate cultures were re-fed with either base medium (serum-free), fresh PCM, or one of the three combinations of differentiation promoting media (all serum-free). After 10 days, differentiation media were removed, cultures were washed 3× with PBS, then re-fed with serum-free base medium containing either 5 mM glucose or 22 mM glucose (final concentration). After 18 hours, the conditioned media were collected and subjected to ELISA analysis with antibodies to either Insulin or C-Peptide (DSL laboratories). FIGS. 13A, 13B and 13C, respectively, show insulin release by nonsubcultured cells, and insulin release and C-peptide release in response to glucose challenge. Since some of the cultures contain insulin, and cells can take insulin up from the medium, production of C-peptide is an important confirmation that the cells are synthesizing insulin de novo from the synthesis and processing of proinsulin. Furthermore, the production of insulin and C-peptide is increased in most cases with increasing glucose concentration, suggesting an islet-like function of cells within these cultures. Note that little insulin or C-peptide is produced in the base medium that contains no DPFs.

### Example 11

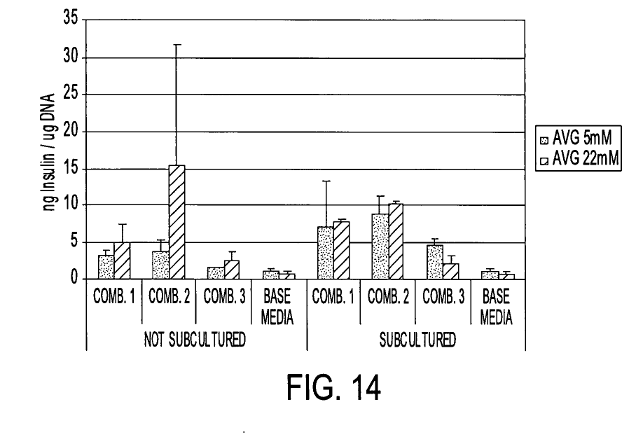

Both the quantity of insulin and the quantity of DNA were measured in IP cells subjected to differentiation culture with or without enzymatic detachment and subculturing. Cultures were carried out precisely as described in the previous paragraph. DNA was measured utilizing a standard Picogreen assay (Molecular Probes), while insulin was measured by ELISA assay. Total ng of Insulin was divided by total μg of DNA in the sample, thus providing the insulin:DNA ratio value, in order to calculate a ratio of the quantity of insulin present vs. the number of cells present (reflected by DNA content). The results are shown in FIG. 14. In each of the differentiation media combinations, the insulin:DNA ratio is increased compared to base medium, suggesting that more insulin is produced on a per cell basis in the presence of DPFs than when cultured without them. Furthermore, the insulin:DNA ratio is increased slightly in some conditions upon glucose challenge (22 mM glucose vs. 5 mM), suggesting that the cells respond to glucose by releasing a greater quantity of insulin.

### Example 12

Insulin-producing cells obtained by the preceding method were subjected to gene expression analysis as described above. Table 5 contains a list of the highest expressed genes, their position on the Clontech atlas 8K gene array, and relative expression of these genes (after normalization). Table 5 is attached hereto as Appendix 1

### Example 13

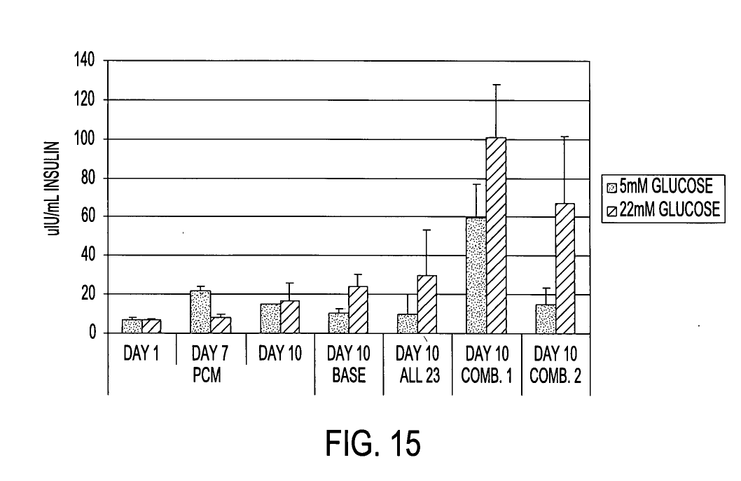

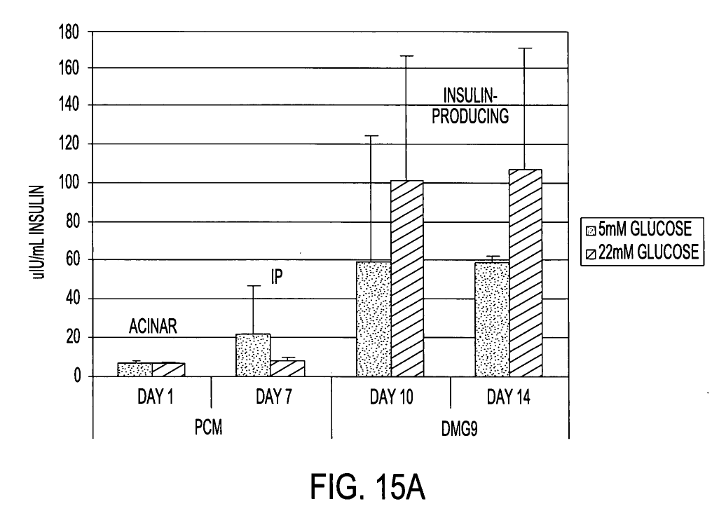

Primary human pancreatic cells were seeded at 0.5×10⁵ cells/cm² in PCM on a collagen-1 surface and grown for 7 days. Insulin was measured at Days 1, 7, and 10 as follows: Growth medium was removed, wells were washed 3× in phosphate buffered saline. After a pre-incubation for 1 hour at 37C in base medium without insulin, with 5 mM glucose, media was removed and replaced with either 1) base medium (without insulin) with 5 mM glucose, or 2) base medium (without insulin) with 22 mM glucose. Insulin was measured in cell-conditioned media after 18 hours at 37° C. After 7 days of culture, PCM medium was replaced with either 1) fresh PCM, 2) serum-free base medium, 3) serum free base medium with all 23 differentiating factors, 4) serum-free combination 1, or 5) serum-free combination 2. The results are shown in FIG. 15. After 3 days exposure to the differentiating factors, increased insulin release is noted in presence of differentiating factors. The results on Day 1 argue against the presence of a significant number of insulin-producing cells in the starting material, demonstrating the de novo generation of insulin-producing cells from acinar cells in the primary culture. It can be seen in the Figure that at the end of 10 days, insulin release in response to a glucose challenge is much greater in the DFP media than in the PCM or base medium, verifying the stimulatory effect that the DFPs exert on transformation of glandular epithelial cells into insulin-producing cells.

### Example 14

Human pancreatic acinar cells were cultured on a collagen I surface in PCM from Day 1 to Day 7, thus generating a culture of IP cells at Day 7. On Day 7, the IP cells were washed and the PCM medium was replaced with the G09 differentiation medium containing the 30 factors listed in Table 2. At each time point (Days 1, 7, 10 and 14), insulin release was measured by washing the cultures three times with PBS, then challenging the cultures with a 1:1 mixture of DMEM and HAMs F12 containing either 5 mM or 22 mM glucose. After 18 hours of exposure to the glucose, supernatants were collected and insulin measured by ELISA. The results are shown in FIG. 15a.

### III. Expression Studies at Several Time Points of Primary Human Acinar Cells that are Expanded, Allowed to Differentiate into IP Cells and then Allowed to Differentiate Further into Insulin-Producing Cells

### Example 15

Three independent samples of primary human pancreatic acinar cells were seeded and expanded described above. From Day 0 to Day 8, cells were on collagen I surface, seeded at 10⁴ cells/cm², in PCM. On Day 8, the medium was changed from PCM to the medium with the active factors shown in Table 2. Cells were fed twice with G09 (50% of medium replaced) between days 8 and 16. The cells remained on the surface throughout the culture process. Cultures were harvested at 3 days after the initial plating (actively trans-differentiating acinar cells), 8 days after plating (IP cells) and 16 days after plating (putative insulin producing cells) and subjected to gene expression analysis, as described in Example 7. mRNA expression data were obtained with 12K microarrays from Clonetech.

Briefly, growth medium was removed from the culture flasks and cells were lysed in trizol LS (Invitrogen) chaotrope/phenol reagent for about 2 minutes by pipetting the lysis solution over the cell layer. Three ml of RNAse free water was added per 9 ml of lysis solution in an Oak Ridge Cetrifuge tube. 2.4 ml chloroform was then added and the solution vigorously vortexed for 1 minute. The aqueous and organic phases were then separated by cetrifugation at 4° C. and the upper aqueous phase containing RNA was removed to a clean PET tube. The RNA was precipitated by isopropanol precipitation, washed with 70% ethanol and redissolved in 200 μl of RNAse free water. A chaotrope lysis reagent was immediately added to the RNA and it was further purified using a Qiagen spin column method with a DNAse digestion step. The purified RNA was finally eluted in 80 μl RNAse free water and stored at −80° C.

Labelled P-33 cDNA probes were prepared from the 30 μg of total RNA from each sample by first enriching for poly A+RNA using a streptavidin-magnetic bead separation method that is part of the Atlas Pure Total RNA Labeeling system. The labeled probes from each sample were hybridized with the plastic human 12 K gene arrays for about 16 hours, the arrays were washed and imaged according to the Atlas array protocols. The Atlas image 2.7 software was used to alighn array images with the array grid template and to exclude false background signals or false signals due to strong signal bleedover. The transcript signals were then extracted from these aligned arrays using the Atlas Image 2.7 software and further statistical analysis of the changes in gene expression were performed.

The raw expression data were analyzed as follows: (1) We filtered out genes that were not expressed at any of the 3 conditions/time points; (2) We normalized all of the microarrays against each other to remove differences from array-to-array and the effects of variability in sample processing, hybridization, etc.; (3) We identified genes which showed a statistically significant difference among the conditions/time points; and (4) We clustered the genes based on their temporal patterns in a way that is consistent with the design of the study and the changes in phenotype.

Table 6 shows expression data for the genes that were identified by the above analysis. This Table is attached hereto as Appendix 2. These identified genes were expressed at high levels at both Day 3 and Day 8, or their expression increased substantially between Day 3 and Day 8. The Table also shows the expression levels of these genes at Day 16, and the mean expression for all three condition/time points. The Table also shows the ratios of expression at various times: “I to A” is the ratio of expression of putative insulin-producing cells (Day 16) to acinar (Day 8) cells; “Int to A” is the ratio of IP cells (Day 8) cells to acinar cells (Day 3).

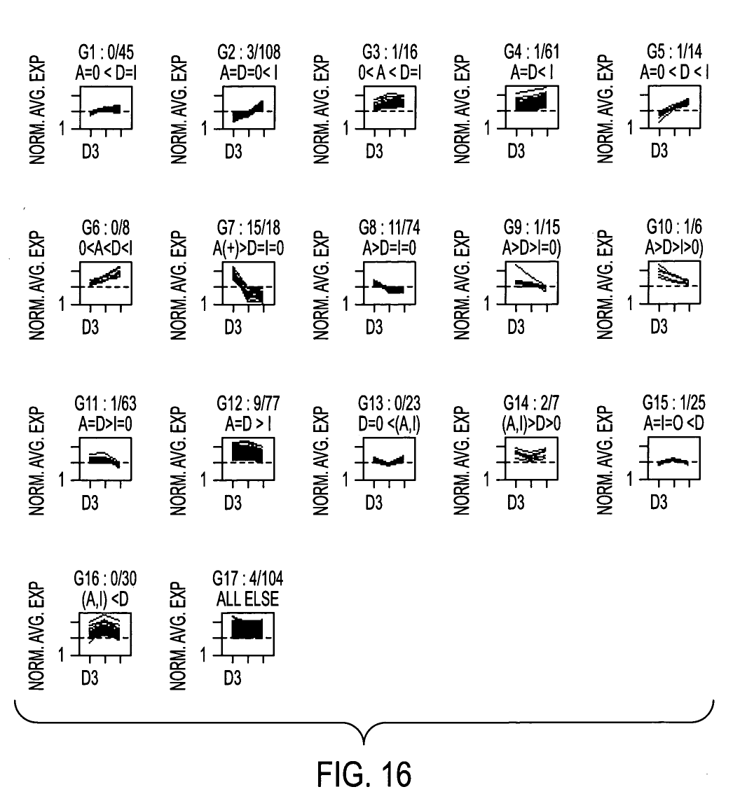

The data shown in Table 6 were further analyzed by clustering them into one of 17 “classes,” whose features are summarized on the Table. A graphical representation of the characteristics of these 17 classes in presented in FIG. 16.

The data from the Day 8 time points in Table 6 were also grouped with regard to whether the genes expressed at Day 8 in these cells belong to the classes of genes expressed normally in (1) liver and pancreas; (2) pancreas-associated genes; (3) liver-associated genes; or (4) progenitor-associated genes. The results are shown in Table 7.

TABLE 7

| Genes Expressed in | BDT Intermediate Cells | | | Hepatic Pattern | Pancreatic Pattern |
|---|---|---|---|---|---|
| Liver and Pancreas | Sample 1 | Sample 2 | Sample 3 | of Expression | of Expression |
| CK18 | + | + | + | hepatic lineage | acinar cells |
| CK8 | + | + | + | hepatic lineage | acinar cells |
| CK19 | + | + | + | bile duct | duct cells |
| CK7 | + | + | + | bile duct | duct cells |
| HNF1 | + | + | + | liver tc factor | in beta cells |
| α-1 antitrypsin | ++ | ++ | ++ | produced in differen- | yes |
| | | | | tiated hepatocytes | |
| Notch-1 | − | − | Trace | involved in differen- | developing |
| | | | | tiation of cells | pancreatic |
| | | | | into hepatic lineage | epithelium |
| α-fetoprotein | − | − | − | developing hepatocytes | developing |
| | | | | and hepatic progenitors | pancreatic ducts |
| Notch-3 | Trace | Trace | Trace | involved in differen- | pancreatic mesenchyme |
| | | | | tiation of cells into | and endothelium |
| | | | | hepatic lineage | |
| Notch-4 | Trace | Trace | Trace- | involved in differen- | pancreatic mesenchyme |
| | | | | tiation of cells into | and endothelium |
| | | | | hepatic lineage | |
| Jagged-2 | + | + | + | involved in differen- | yes |
| | | | | tiation of cells into | |
| | | | | hepatic lineage | |
| pi-glutathione s | + | + | ++ | developing liver | duct cells and |
| transferase (pi-GST) | | | | centroacinar cells | |
| γ-glutamyl | − | − | − | developing bile duct | acinar cells |
| transferase | | | | | |
| Pancreas-Associated | | | | Hepatic Pattern | Pancreatic Pattern |
| Genes | Sample 1 | Sample 2 | Sample 3 | of Expression | of Expression |
| carbonic anhydrase | − | − | Trace | no | duct cells |
| CF transmemb conductance | − | − | Trace | no | duct cells |
| regulator | | | | | |
| elastase | − | − | − | no | acinar cells |
| amylase | − | − | − | no | acinar cells |
| insulin | + | Trace | Trace | no | islet |
| somatostatin | Trace | Trace | Trace | no | islet |
| Pancreatic Polypeptide | + | + | Trace | no | islet |
| Glucagon | − | − | − | no | Islet |
| | | | | Hepatic Pattern | Pancreatic Pattern |
| Liver-Associated Genes | Sample 1 | Sample 2 | Sample 3 | of Expression | of Expression |
| Sialyltransferase-6 | − | − | − | produced in differen- | no |
| | | | | tiated hepatocytes | |
| Liver-specific bHLH | + | + | + | liver-specific Tc | no |
| transcrip factor | | | | factor | |
| Thy-1 | + | + | + | hepatic oval cell | no |
| | | | | marker | |
| Glucose-6-phosphatase | − | − | Trace | hepatic lineage, | No |
| | | | | progenitors and adult | |
| Glutamine synthetase | − | − | Trace | hepatocyte | No |
| Carbamoyl phosphate | − | − | − | hepatocyte | no |
| synthetase-1 | | | | | |
| Dipeptidylpeptidase IV | − | − | − | hepatocyte | no |
| C/EBP-α | ++ | ++ | + | liver-specific | *turned on and upregul |
| | | | | Tc factor | during hepatization of |
| | | | | | pancreas (copper- |
| | | | | | deficient diet) |
| C/EBP-beta | ++ | ++ | ++ | liver-specific | *turned on and upregul |
| | | | | Tc factor | during hepatization of |
| | | | | | pancreas (copper- |
| | | | | | deficient diet) |

| Progenitor Cell-Associated Genes | Sample 1 | Sample 2 | Sample 3 | Tissue |
|---|---|---|---|---|
| Musashi-1 | − | − | − | Intestine |
| Nestin | − | − | − | Pancreas/Neuronal |
| CD34 | − | − | − | Hematopoietic |
| Thy-1 | − | − | − | Hepatic progenitors |
| BMP-2 | − | − | − | Neuronal |
| BMPRcIA | − | − | Trace | Mesenchyme |
| c-kit | − | − | − | Liver/Pancreas/Neuronal |
| chromogranin A | Trace | Trace | Trace | Neuroendocrine/Liver/Intestine |
| PDX-1 | − | − | + | Pancreas |

As can be seen, at Day 8 IP cells no longer expressed genes consistent with pancreatic acinar cells, nor did they express a complement of genes specific for pancreatic ductular cells. The IP cells expressed low levels of some markers associated with pancreatic islets, including insulin, somatostatin and pancreatic polypeptide, suggesting that at least some cells in the population are competent to express endocrine genes of the pancreatic islets.

Surprisingly, the IP cells also expressed several liver-specific transcription factors (e.g., C/EBP alpha, C-EBP-beta) and other markers of mature and developing liver, including low levels of Thy-1, a marker associated with hepatic “oval” stem cells. This suggests that the differentiating cells were not moving simply from pancreatic acinar to pancreatic ductal, but had developed into a cell with both hepatic and pancreatic characteristics, while not fitting into any single gene expression profile of one of these cell types. The cells generated in this example resemble the cells that emerge from the pancreas of rodents that are fed a copper-deficient diet. (See, e.g. Rao et al., 1988). The pancreas of such animals goes through an acute phase of pancreatitis followed by “hepatization” of liver (which means cells that begin to express hepatic genes rather than pancreatic genes). Liver-like cells have also been reported in human fetal pancreas (Tsanadis et al., 1995) Isolated cells generated by the methods of the present invention (e.g., by propagating primary acinar cells or other types of endodermal cells or progenitor cells by the methods of the invention) are to be distinguished from naturally occurring cells that may have some of the characteristics of IP cells, such as oval cells or cells isolated from the pancreas of a rodent on a copper-deficient diet.

Cells having the characteristics of these IP cells may be useful for, e.g., therapeutic approaches in the treatment of diabetes. Furthermore, although the cells in this example were derived from pancreas, other epithelial tissues, or perhaps even any endoderm-derived tissue, may provide additional sources of cells that can be differentiated into cells having a similar phenotype. Suitable tissue types include, e.g., liver or intestine. These IP cells express genes associated with pancreas, liver, intestine and neuronal tissues. For example, they express mucin, CK19 and CK7, which are common markers associated with duct cells in the pancreas, liver and intestine. Thus, the gene expression pattern seen in these IP cells may serve as a predictive measure for cells derived from each of these tissues for the purpose of generating insulin-producing cells. Furthermore, IP cells may, under appropriate conditions, give rise, not only to pancreatic islet cells, but also to hepatocytes or any endoderm-derived tissue.

The disclosures of the following references, cited above in part, relate to the present invention:

WO 02/29010 A2 (Kerr-Conte);

Bonner-Weir, S. et al., Proc. Natl. Acad. Sci. USA 97: 7999-8004 (2000),

Bouwens, L., Microsc. Res. Tech. 43: 332-6 (1998),

Bowens, L. et al. Diabetologia 41:629-33 (1998);

Gmyr, V. et al., Diabetes 49:1671-80 (2000);

Gmyr, V. et al. Cell Transplant 10:109-21 (2001),

Gmyr, V. et al. Diabetes 49:1671-80 (2000),

Hall, P. A. et al., J. Pathol. 166: 97-103 (1992);

Kerr-Conte, J. et al., Diabetes 45:1108-14 (1996);

Kerr-Conte, J. et al., Transplant Proc 27:3268 (1985);

Pattou F. et al., Bull. Acad. Natl. Med. 184:1887-99 (2000);

Rao, M S et al Biochem Biophys Res Comm. 156:131-6 (1988);

Rooman, Ilse et al., Diabetes 51: 686-90 (2002);

Rooman, I, et al. Diabetologia 43:907-14 (2000);

Rooman, I. et al., Gastroenterology 121: 940-9 (2001);

Trivedi, N. et al. Endocrinology 142:2115-22 (2001);

Tsanadis, G. et al. Histol. Histopathol. 10:1-10 (1995);

Wang, R. N. et al., Diabetologia 38:1405-11(1995);

U.S. Pat. No. 6,011,647 (Ammon Peck).

The embodiments illustrated and discussed in the present specification are intended only to teach those skilled in the art the best way known to the inventors to make and use the invention, and should not be considered as limiting the scope of the present invention. The exemplified embodiments of the invention may be modified or varied, and elements added or omitted, without departing from the invention, as appreciated by those skilled in the art in light of the above teachings. It is therefore to be understood that, within the scope of the claims and their equivalents, the invention may be practiced otherwise than as specifically described.

The entire disclosure of all applications, patents and publications, cited above and in the figures are hereby incorporated in their entirety by reference.

TABLE 5
APPENDIX 1:

| | | Relative | Relative |
|---|---|---|---|
| | Position on | Expression, | Expression, |
| GENE | Atlas 8K Array | Sample 1 | Sample 2 |
| aquaporin 5 | 7547 | 4539 | 4550 |
| actin, beta | 3952 | 3598 | 3442 |
| actin, beta | 8176 | 3047 | 3747 |
| growth hormone secretagogue receptor | 6846 | 2092 | 1710 |
| profilin 1 | 448 | 1580 | 1246 |
| special AT-rich sequence binding protein 1 | 3035 | 1437 | 886 |
| (binds to nuclear matrix/scaffold- | | | |
| associating DNA's) | | | |
| cardiac-specific homeo box | 2050 | 1395 | 2850 |
| CCAAT/enhancer binding protein (C/EBP), | 1588 | 1374 | 1671 |
| beta | | | |
| RAS guanyl releasing protein 2 (calcium | 8262 | 1263 | 1616 |
| and DAG-regulated) | | | |
| cartilage paired-class homeoprotein 1 | 6677 | 1166 | 1241 |
| paired-like homeodomain transcription | 6805 | 1113 | 756 |
| factor 1 | | | |
| transcription factor 21 | 7621 | 1063 | 801 |
| CD3E antigen, epsilon polypeptide (TiT3 | 8054 | 994 | 1113 |
| complex) | | | |
| CD151 antigen | 5613 | 935 | 808 |
| ATPase, Ca++ transporting, plasma | 7940 | 919 | 876 |
| membrane 2 | | | |
| ficolin (collagen/fibrinogen domain- | 824 | 901 | 829 |
| containing) 3 (Hakata antigen) | | | |
| inositol polyphosphate phosphatase-like 1 | 5989 | 832 | 425 |
| protein tyrosine phosphatase, receptor | 8367 | 806 | 810 |
| type, S | | | |
| integrin, alpha 3 (antigen CD49C, alpha 3 | 1743 | 758 | 647 |
| subunit of VLA-3 receptor) | | | |
| syntaxin 1A (brain) | 6851 | 743 | 658 |
| parathymosin | 7135 | 725 | 852 |
| thymosin, beta 10 | 7436 | 709 | 1617 |
| midkine (neurite growth-promoting factor 2) | 2470 | 682 | 802 |
| arylsulfatase A | 3147 | 678 | 3084 |
| calbindin 2, (29 kD, calretinin) | 7000 | 676 | 786 |
| serine (or cysteine) proteinase inhibitor, | 5214 | 668 | 678 |
| clade A (alpha-1 antiproteinase, | | | |
| antitrypsin), member 1 | | | |
| retinal G protein coupled receptor | 7972 | 653 | 696 |
| myosin regulatory light chain 2, smooth | 2499 | 638 | 561 |
| muscle isoform | | | |
| butyrate response factor 1 (EGF-response | 7325 | 630 | 646 |
| factor 1) | | | |
| type I transmembrane receptor (seizure- | 7583 | 589 | 394 |
| related protein) | | | |
| type I transmembrane receptor (seizure- | 7583 | 589 | 394 |
| related protein) | | | |
| procollagen C-endopeptidase enhancer | 3593 | 576 | 713 |
| mitogen-activating protein kinase kinase | 7069 | 558 | 571 |
| kinase kinase 2 | | | |
| protease, serine, 1 (trypsin 1) | 6323 | 558 | 656 |
| pancreatitis-associated protein | 4576 | 536 | 2177 |
| angiotensin receptor 1B | 4093 | 516 | 491 |
| stratifin | 6489 | 503 | 581 |
| keratin 17 | 7903 | 500 | 364 |
| somatostatin receptor 3 | 6872 | 432 | 433 |
| myosin-binding protein H | 4692 | 428 | 96 |
| ephrin-A5 | 4202 | 411 | 381 |
| RAP1, GTPase activating protein 1 | 1965 | 407 | 263 |
| chymotrypsinogen B1 | 6604 | 405 | 830 |
| ankyrin-like with transmembrane domains 1 | 3577 | 395 | 431 |
| Misshapen/NIK-related kinase | 2439 | 391 | 341 |
| bone morphogenetic protein 6 | 4492 | 381 | 308 |
| splicing factor proline/glutamine rich | 713 | 375 | 662 |
| (polypyrimidine tract-binding protein- | | | |
| associated) | | | |
| claudin 12 | 2720 | 370 | 378 |
| matrix metalloproteinase 23A | 7491 | 368 | 138 |
| neurogranin (protein kinase C substrate, | 4465 | 366 | 335 |
| RC3) | | | |
| dopamine receptor D2 | 1649 | 362 | 234 |
| cardiotrophin 1 | 5283 | 359 | 507 |
| CCAAT/enhancer binding protein (C/EBP), | 7237 | 359 | 645 |
| alpha | | | |
| paired box gene 9 | 5206 | 335 | 201 |
| protein tyrosine phosphatase, receptor | 8235 | 331 | 250 |
| type, N | | | |
| keratin 8 | 7215 | 327 | 449 |
| claudin 7 | 280 | 325 | 173 |
| trophinin associated protein (tastin) | 462 | 323 | 360 |
| neuronal thread protein | 8356 | 322 | 366 |
| basic helix-loop-helix domain containing, | 6734 | 318 | 215 |
| class B, 2 | | | |
| annexin A2 | 4467 | 290 | 226 |
| cathepsin D (lysosomal aspartyl protease) | 7370 | 289 | 600 |
| Bicaudal D (Drosophila) homolog 1 | 6822 | 284 | 217 |
| lectin, galactoside-binding, soluble, 1 | 7331 | 280 | 310 |
| (galectin 1) | | | |
| keratin 7 | 7171 | 278 | 481 |
| glyceraldehyde-3-phosphate | 3953 | 278 | 307 |
| dehydrogenase | | | |
| aquaporin 6, kidney specific | 7591 | 277 | 258 |
| TNF receptor-associated factor 1 | 6037 | 269 | 270 |
| Rho GDP dissociation inhibitor (GDI) alpha | 5565 | 267 | 321 |
| calcium channel, voltage-dependent, | 3142 | 260 | 207 |
| gamma subunit 4 | | | |
| glutamate receptor, ionotropic, kainate 1 | 4776 | 244 | 296 |
| CCR4-NOT transcription complex, subunit 4 | 983 | 244 | 428 |
| filamin A, alpha (actin-binding protein-280) | 3457 | 241 | 233 |
| peanut (Drosophila)-like 1 | 711 | 233 | 310 |
| enhancer of rudimentary (Drosophila) | 5921 | 231 | 405 |
| homolog | | | |
| endothelin converting enzyme 1 | 7835 | 229 | 197 |
| protocadherin 17 | 2483 | 223 | 208 |
| aquaporin 8 | 7679 | 217 | 145 |
| synapsin I | 4871 | 216 | 164 |
| Tubulin, alpha, brain-specific | 2827 | 212 | 345 |
| CD44 antigen (homing function and Indian | 7848 | 207 | 141 |
| blood group system) | | | |
| cerebral cavernous malformations 1 | 2731 | 205 | 116 |
| insulin-like 3 (Leydig cell) | 666 | 202 | 365 |
| adenylate cyclase activating polypeptide 1 | 4489 | 200 | 74 |
| (pituitary) | | | |
| hairless (mouse) homolog | 5185 | 196 | 36 |
| insulin | 4427 | 190 | 44 |
| granulin | 928 | 184 | 201 |
| granulin | 928 | 184 | 201 |
| early growth response 1 | 2111 | 178 | 136 |
| cyclin-dependent kinase inhibitor 1A (p21, | 7852 | 175 | 108 |
| Cip1) | | | |
| stem cell growth factor; lymphocyte | 1606 | 174 | 152 |
| secreted C-type lectin | | | |
| CD4 antigen (p55) | 5608 | 172 | 143 |
| PCTAIRE protein kinase 1 | 6334 | 161 | 78 |
| Rho GTPase activating protein 6 | 1035 | 160 | 220 |
| G protein-coupled receptor 37 (endothelin | 6318 | 158 | 35 |
| receptor type B-like) | | | |
| syndecan 4 (amphiglycan, ryudocan) | 3154 | 157 | 147 |
| PCTAIRE protein kinase 3 | 8050 | 155 | 159 |
| empty spiracles (Drosophila) homolog 2 | 2084 | 150 | 207 |
| transglutaminase 1 (K polypeptide | 6674 | 150 | 101 |
| epidermal type I, protein-glutamine-gamma | | | |
| glutamyltransferase) | | | |
| potassium voltage-gated channel, | 1335 | 147 | 62 |
| subfamily G, member 2 | | | |
| aldehyde dehydrogenase 4 (glutamate | 5985 | 147 | 207 |
| gamma-semialdehyde dehydrogenase; | | | |
| pyrroline-5-carboxylate dehydrogenase) | | | |
| E1A binding protein p300 | 1433 | 144 | 197 |
| LIM homeobox transcription factor 1, beta | 8211 | 143 | 89 |
| eukaryotic translation elongation factor 2 | 4536 | 134 | 150 |
| mitogen-activated protein kinase kinase | 5462 | 129 | 92 |
| kinase 10 | | | |
| PPAR(gamma) angiopoietin related protein | 3074 | 129 | 56 |
| homeo box A5 | 7037 | 128 | 87 |
| CD63 antigen (melanoma 1 antigen) | 7769 | 124 | 91 |
| nuclear receptor coactivator 3 | 4181 | 119 | 84 |
| CD68 antigen | 443 | 118 | 256 |
| transmembrane 4 superfamily member 7 | 1571 | 117 | 29 |
| pancreatic polypeptide | 2735 | 116 | 136 |
| endothelin type b receptor-like protein 2 | 6656 | 114 | 74 |
| neurogenin 1 | 5772 | 113 | 91 |
| insulin receptor substrate 2 | 6016 | 110 | 130 |
| glutamate receptor, ionotropic, N-methyl D- | 4702 | 109 | 58 |
| asparate-associated protein 1 (glutamate | | | |
| binding) | | | |
| neurotrophin 5 (neurotrophin 4/5) | 8259 | 108 | 89 |
| pyrimidinergic receptor P2Y, G-protein | 3857 | 105 | 65 |
| coupled, 6 | | | |
| empty spiracles (Drosophila) homolog 1 | 2040 | 103 | 87 |
| chromobox homolog 2 (Drosophila Pc | 2832 | 101 | 139 |
| class) | | | |
| heart and neural crest derivatives | 2146 | 92 | 129 |
| expressed 1 | | | |
| transglutaminase 2 (C polypeptide, protein- | 7731 | 92 | 74 |
| glutamine-gamma-glutamyltransferase) | | | |
| enolase 1, (alpha) | 1389 | 92 | 205 |
| thyroid transcription factor 1 | 4519 | 89 | 60 |
| keratin 19 | 5923 | 88 | 223 |
| Deleted in split-hand/split-foot 1 region | 3641 | 87 | 104 |
| glutathione peroxidase 4 (phospholipid | 1874 | 84 | 72 |
| hydroperoxidase) | | | |
| aristaless (Drosophila) homeobox | 1783 | 81 | 103 |
| GATA-binding protein 4 | 659 | 80 | 110 |
| transcription factor 1, hepatic; LF-B1, | 712 | 80 | 136 |
| hepatic nuclear factor (HNF1), albumin | | | |
| proximal factor | | | |
| heat shock transcription factor 1 | 6708 | 79 | 57 |
| liver-specific bHLH-Zip transcription factor | 765 | 77 | 188 |
| liver-specific bHLH-Zip transcription factor | 765 | 77 | 188 |
| eukaryotic translation initiation factor 3, | 6280 | 76 | 94 |
| subunit 4 (delta, 44 kD) | | | |
| eukaryotic translation initiation factor 3, | 6280 | 76 | 94 |
| subunit 4 (delta, 44 kD) | | | |
| gamma-aminobutyric acid (GABA) A | 3720 | 70 | 178 |
| receptor, alpha 6 | | | |
| retinoic acid receptor, gamma | 5191 | 70 | 109 |
| homeo box D9 | 1776 | 69 | 34 |
| MAD1 (mitotic arrest deficient, yeast, | 1946 | 63 | 38 |
| homolog)-like 1 | | | |
| homeo box A4 | 7177 | 57 | 103 |
| Thy-1 cell surface antigen | 559 | 57 | 87 |
| talin | 603 | 53 | 84 |
| bone morphogenetic protein receptor, type | 332 | 53 | 45 |
| II (serine/threonine kinase) | | | |
| hepatocyte nuclear factor 3, alpha | 72 | 52 | 60 |
| ryanodine receptor 2 (cardiac) | 5244 | 47 | 40 |
| signal transducer and activator of | 3661 | 40 | 35 |
| transcription 6, interleukin-4 induced | | | |

APPENDIX 2: TABLE 6

| | | | | | | | | | |
|---|---|---|---|---|---|---|---|---|---|
| | | | | | | | | rationIntto/ | |
| SwissProt | Spot | geneName | day 3 | day 8 | day 16 | allMean | ratioItoA | classID | className |

| | | | | | | | | | | | |
|---|---|---|---|---|---|---|---|---|---|---|---|
| P04270 | E19ab2 | actin: alpha: cardiac | 1198.174 | 760 | 5955.077 | 2637.676 | 2.31E+00 | −6.57E−01 | 7 | Equal A/Int; | |
| | | muscle | | | | | | | | Up Islets | |
| O95996 | N17ab2 | adenomatous polyposis | 2708.542 | 5180 | 7594.139 | 5161.464 | 1.49E+00 | 9.36E−01 | 7 | Equal A/Int; | |
| | | coli like | | | | | | | | Up Islets | |
| Q14697 | G02ef8 | alpha glucosidase II | 306.0691 | 350 | 775.3358 | 477.0123 | 1.34E+00 | 1.92E−01 | 7 | Equal A/Int; | |
| | | alpha subun | | | | | | | | Up Islets | |
| P14209 | G23ab7 | antigen identified by | 389.822 | 507 | 1238.43 | 711.8924 | 1.67E+00 | 3.80E−01 | 7 | Equal A/Int; | |
| | | monoclonal | | | | | | | | Up Islets | |
| P30530 | G14ef5 | AXL receptor tyrosine | 314.4639 | 288 | 634.5658 | 412.2115 | 1.01E+00 | −1.29E−01 | 7 | Equal A/Int; | |
| | | kinase | | | | | | | | Up Islets | |
| O43770 | I23ab3 | B-cell CLL/lymphoma | 700.573 | 1050 | 1504.52 | 1085.931 | 1.10E+00 | 5.87E−01 | 7 | Equal A/Int; | |
| | | 7C | | | | | | | | Up Islets | |
| O43852 | P08ab3 | calumenia | 801.582 | 1290 | 4720.807 | 2269.921 | 2.56E+00 | 6.84E−01 | 7 | Equal A/Int; | |
| | | | | | | | | | | Up Islets | |
| O75718 | O24cd6 | cartilage associated | 430.4432 | 256 | 1090.59 | 592.2327 | 1.34E+00 | −7.52E−01 | 7 | Equal A/Int; | |
| | | protein | | | | | | | | Up Islets | |
| Q9Y3C0 | G14ef2 | CGI-116 protein | 181.0293 | 301 | 416.7246 | 299.7237 | 1.20E+00 | 7.36E−01 | 7 | Equal A/tat: | |
| | | | | | | | | | | Up Islets | |
| P78369 | G09cd6 | claudin 10 | 1176.087 | 742 | 2598.595 | 1505.471 | 1.14E+00 | −6.65E−01 | 7 | Equal A/Int; | |
| | | | | | | | | | | Up Islets | |
| P08572 | P08ef6 | collagen: type IV: | 1130.23 | 1350 | 4684.425 | 2389.488 | 2.05E+00 | 2.60E−01 | 7 | Equal A/Int; | |
| | | alpha 2 | | | | | | | | Up Islets | |
| P11802 | B05ef5 | cyclin-dependent | 914.1579 | 978 | 2342.994 | 1411.839 | 1.36E+00 | 9.79E−02 | 7 | Equal A/Int; | |
| | | kinase 4 | | | | | | | | Up Islets | |
| Q16555 | O07ab4 | dihydropyrimidinase- | 114.6165 | 164 | 921.1473 | 399.8267 | 3.01E+00 | 5.14E−01 | 7 | Equal A/Int; | |
| | | like 2 | | | | | | | | Up Islets | |
| P33316 | H23ab5 | dUTP | 177.2032 | 238 | 541.5382 | 318.7836 | 1.61E+00 | 4.23E−01 | 7 | Equal A/Int; | |
| | | pyropttosphatase | | | | | | | | Up Islets | |
| P07992 | K20ef6 | excision repair | 362.031 | 415 | 907.7933 | 561.6388 | 1.33E+00 | 1.97E−01 | 7 | Equal A/Int; | |
| | | cross-complemen | | | | | | | | Up Islets | |
| O75636 | B14cd3 | ficolin (collagen/ | 1081.127 | 1010 | 4327.806 | 2139.448 | 2.00E+00 | −9.87E−02 | 7 | Equal A/Int; | |
| | | fibrinogen domai | | | | | | | | Up Islets | |
| Q9UBA6 | B19ef4 | G8 protein | 370.6842 | 578 | 1489.206 | 812.6378 | 2.01E+00 | 6.41E−01 | 7 | Equal A/Int; | |
| | | | | | | | | | | Up Islets | |
| Q9Y5P5 | P08ef7 | GDP-mannose | 171.1216 | 198 | 414.0862 | 261.0679 | 1.27E+00 | 2.10E−01 | 7 | Equal A/Int; | |
| | | pyrophosphorylas | | | | | | | | Up Islets | |
| O75293 | E02ef7 | growth arrest and | 194.3626 | 203 | 386.6833 | 261.3841 | 9.92E−01 | 6.35E−02 | 7 | Equal A/Int; | |
| | | DNA-damage-i | | | | | | | | Up Islets | |
| P50152 | C05ab6 | guanine nucleotide | 204.743 | 150 | 497.3097 | 283.8702 | 1.28E+00 | −4.53E−01 | 7 | Equal A/Int; | |
| | | binding prote | | | | | | | | Up Islets | |
| Q9NX09 | C05gh3 | HIF-1 responsive | 348.8574 | 253 | 1032.929 | 545.0622 | 1.57E+00 | −4.61E−01 | 7 | Equal A/Int; | |
| | | RTP801 | | | | | | | | Up Islets | |
| P05204 | J22ab7 | high-mobility group | 2067.865 | 1120 | 6059.895 | 3082.818 | 1.55E+00 | −8.84E−01 | 7 | Equal A/Int; | |
| | | (nonhistone | | | | | | | | Up Islets | |
| Q9P0P2 | C17ef3 | homolog of yeast | 325.8407 | 378 | 682.7568 | 462.1583 | 1.07E+00 | 2.14E−01 | 7 | Equat A/Int; | |
| | | MOG1 | | | | | | | | Up Islets | |
| Q9NWF8 | L15gh2 | hypothetical protein | 172.167 | 241 | 374.3 | 262.6222 | 1.12E+00 | 4.88E−01 | 7 | Equal A/Int; | |
| | | FLJ10055 | | | | | | | | Up Islets | |
| Q9NWV4 | B17gh2 | hypothetical protein | 221.4432 | 280 | 483.5381 | 328.2481 | 1.13E+00 | 3.37E−01 | 7 | Equal A/Int; | |
| | | FLJ20580 | | | | | | | | Up Islets | |
| Q9P0S8 | A21ef3 | hypothetical protein | 153.824 | 165 | 330.9111 | 216.4274 | 1.11E+00 | 9.72E−02 | 7 | Equal A/Int; | |
| | | HSPC195 | | | | | | | | Up Islets | |
| Q9BWS9 | P04gh5 | hypothetical protein | 196.9154 | 161 | 427.2066 | 261.7132 | 1.12E+00 | −2.90E−01 | 7 | Equal A/Int; | |
| | | MGC3234 | | | | | | | | Up Islets | |
| Q9BSK0 | K21gh8 | hypothetical protein | 156.4692 | 117 | 684.3425 | 319.1224 | 2.13E+00 | −4.25E−01 | 7 | Equal A/Int; | |
| | | MGC4415 | | | | | | | | Up Islets | |
| Q9Y6M1 | P09cd6 | IGF-II mRNA-biading | 135.2566 | 153 | 408.5101 | 232.2937 | 1.59E+00 | 1.79E−01 | 7 | Equal A/Int; | |
| | | protein 2 | | | | | | | | Up Islets | |
| Q9NQX7 | H02gh7 | integral membrane | 478.2728 | 481 | 1102.244 | 687.2575 | 1.20E+00 | 8.97E−03 | 7 | Equal A/Int; | |
| | | protein 3 | | | | | | | | Up Islets | |
| P08648 | E23ef7 | integrin: alpha 5 | 183.4629 | 124 | 478.7266 | 282.0713 | 1.38E+00 | −5.65E−01 | 7 | Equal A/Int; | |
| | | (fibronectin rece | | | | | | | | Up Islets | |
| O00410 | J24ab6 | karyopherin (importin) | 326.6874 | 196 | 629.7656 | 384.2402 | 9.47E−01 | −7.35E−01 | 7 | Equal A/Int; | |
| | | beta 3 | | | | | | | | Up Islets | |
| P07195 | N16ab6 | lactate dehydrogenase | 1003.971 | 1200 | 5593.079 | 2599.05 | 2.48E+00 | 2.57E−01 | 7 | Equal A/Int; | |
| | | B | | | | | | | | Up Islets | |
| P09382 | N22ab6 | lectin: galactoside- | 19170.52 | 35400 | 78086.75 | 44205.77 | 2.03E+00 | 8.83E−01 | 7 | Equal A/Int; | |
| | | binding: solub | | | | | | | | Up Islets | |
| O75427 | P20ab6 | leucine-rich repeat | 174.9605 | 231 | 543.3883 | 316.5457 | 1.83E+00 | 4.03E−01 | 7 | Equal A/Int; | |
| | | protein: neur | | | | | | | | Up Islets | |
| Q16553 | A15ab7 | lymphocyte antigen | 425.8933 | 494 | 938.4504 | 619.3701 | 1.14E+00 | 2.13E−01 | 7 | Equal A/Int; | |
| | | 6 complex: I | | | | | | | | Up Islets | |
| O75900 | O05cd4 | matrix melallo- | 381.2184 | 297 | 851.7221 | 510.1038 | 1.16E+00 | −3.58E−01 | 7 | Equal A/Int; | |
| | | proteinase 23A | | | | | | | | Up Islets | |
| Q9UNF1 | H16ef7 | melanoma antigen: | 193.8125 | 219 | 659.9701 | 357.8132 | 1.77E+00 | 1.77E−01 | 7 | Equal A/Int; | |
| | | family D; 2 | | | | | | | | Up Islets | |
| P13995 | J16cd6 | methylene tetra- | 321.4878 | 226 | 759.6021 | 435.6746 | 1.24E+00 | −5.09E−01 | 7 | Equal A/Int; | |
| | | hydrofolate dehy | | | | | | | | Up Islets | |
| O00265 | G19cd8 | microtubule- | 189.7616 | 234 | 377.5576 | 267.156 | 9.93E−01 | 3.03E−01 | 7 | Equal A/Int; | |
| | | associated protein: | | | | | | | | Up Islets | |
| P27361 | B13ef5 | mitogen-activated | 358.0872 | 382 | 720.3119 | 486.849 | 1.01E+00 | 9.38E−02 | 7 | Equal A/Int; | |
| | | protein kinase | | | | | | | | Up Islets | |
| P24844 | E18cd6 | myosin: light poly- | 5480.451 | 7380 | 22518.95 | 11793.09 | 2.04E+00 | 4.29E−01 | 7 | Equal A/Int; | |
| | | peptide 9: regul | | | | | | | | Up Islets | |
| Q9NVD4 | J10gh2 | N-acetylneuraminic | 153.4762 | 167 | 390.6642 | 237.1908 | 1.35E+00 | 1.26E−01 | 7 | Equal A/Int; | |
| | | acid phospha | | | | | | | | Up Islets | |
| Q9Y617 | N22ef8 | phosphoserine amino- | 169.9503 | 250 | 1238.851 | 553.0005 | 2.87E+00 | 5.58E−01 | 7 | Equal A/Int; | |
| | | transferase | | | | | | | | Up Islets | |
| O95356 | A11gh7 | pituitary tumor- | 237.2126 | 192 | 2276.181 | 901.8229 | 3.26E+00 | −3.05E−01 | 7 | Equal A/Int; | |
| | | transforming 3 | | | | | | | | Up Islets | |
| Q9Y5X6 | G04cd6 | plasma glutamate | 169.6268 | 142 | 366.6202 | 226.0168 | 1.11E+00 | −2.58E−01 | 7 | Equal A/Int; | |
| | | carboxypeptid | | | | | | | | Up Islets | |
| Q15113 | G20ab8 | procollagen C- | 497.3967 | 402 | 2628.947 | 1176.048 | 2.40E+00 | −3.08E−01 | 7 | Equal A/Int; | |
| | | endopeptidase en | | | | | | | | Up Islets | |
| O15460 | D20cd4 | procollagen-proline: | 262.1395 | 433 | 756.4003 | 483.9569 | 1.53E+00 | 7.25E−01 | 7 | Equal A/Int; | |
| | | 2-oxoglutara | | | | | | | | Up Islets | |
| O43556 | I19gh1 | sarcoglycan: epsilon | 172.7191 | 215 | 349.304 | 245.5263 | 1.02E+00 | 3.13E−01 | 7 | Equal A/Int; | |
| | | | | | | | | | | Up Islets | |
| P50454 | M17ab6 | serine (or cysteine) | 339.1762 | 612 | 4091.161 | 1680.942 | 3.59E+00 | 8.53E−01 | 7 | Equal A/Int; | |
| | | proteinase in | | | | | | | | Up Islets | |
| Q92853 | A23cd8 | similar to vaccinia | 444.062 | 553 | 997.4551 | 664.6951 | 1.17E+00 | 3.15E−01 | 7 | Equal A/Int; | |
| | | virus -HindIII K | | | | | | | | Up Islets | |
| Q16658 | P20cd1 | singed-like (fascin | 262.7872 | 479 | 1472.155 | 738.014 | 2.49E+00 | 8.66E−01 | 7 | Equal A/Int; | |
| | | homolog: sea | | | | | | | | Up Islets | |
| Q9Y4Y8 | E18cd7 | Sm protein F | 257.2026 | 278 | 548.9705 | 361.3772 | 1.09E+00 | 1.12E−01 | 7 | Equal A/Int; | |
| | | | | | | | | | | Up Islets | |
| Q9BVH9 | D18gh7 | thioredoxin related | 522.0262 | 481 | 1903.964 | 968.9954 | 1.87E+00 | −1.18E−01 | 7 | Equal A/Int; | |
| | | protein | | | | | | | | Up Islets | |
| Q9HBB0 | L08gh8 | Thy-1 co-transcribed | 327.3502 | 374 | 1110.527 | 603.8101 | 1.76E+00 | 1.90E−01 | 7 | Equal A/Int; | |
| | | | | | | | | | | Up Islets | |
| P01033 | P09ef7 | tissue inhihitor of | 1236.457 | 1500 | 13029.41 | 5256.861 | 3.40E+00 | 2.83E−01 | 7 | Equal A/Int; | |
| | | metalloproteina | | | | | | | | Up Islets | |
| Q15582 | L20ef6 | transforming growth | 1221.083 | 2060 | 8540.86 | 3939.09 | 2.81E+00 | 7.51E−01 | 7 | Equal A/Int; | |
| | | factor; beta-i | | | | | | | | Up Islets | |
| Q01995 | M07cd2 | transgelin | 3307.821 | 3880 | 18534.27 | 8573.074 | 2.49E+00 | 2.29E−01 | 7 | Equal A/Int; | |
| | | | | | | | | | | Up Islets | |
| Q13641 | B05ab2 | trophoblast | 479.6488 | 426 | 1399.786 | 768.6158 | 1.55E+00 | −1.70E−01 | 7 | Equal A/Int; | |
| | | glycoprotein | | | | | | | | Up Islets | |
| P06468 | G08cd2 | tropomyosin 2 | 339.2292 | 589 | 13203.05 | 4710.469 | 5.28E+00 | 7.96E−01 | 7 | Equal A/Int; | |
| | | (beta) | | | | | | | | Up Islets | |
| P51784 | M21cd4 | ubiquitin specific | 158.0478 | 194 | 491.1246 | 280.9819 | 1.64E+00 | 2.94E−01 | 7 | Equal A/Int; | |
| | | protease 11 | | | | | | | | Up Islets | |
| P03996 | E17ab2 | actin; alpha 2; | 412.1187 | 1940 | 41801.34 | 14717.63 | 6.66E+00 | 2.23E+00 | 9 | Increasing | |
| | | smooth muscle; a | | | | | | | | A/Int/l | |
| P08123 | P02ef6 | collagen: type I; | 186.5666 | 576 | 14565.36 | 5109.251 | 6.29E+00 | 1.63E+00 | 9 | Increasing | |
| | | alpha 2 | | | | | | | | A/Int/l | |
| P24821 | A05ef7 | hexabrachion | 355.495 | 918 | 2371.983 | 1215.165 | 2.74E+00 | 1.37E+00 | 9 | Increasing | |
| | | (tenascin C: cytota | | | | | | | | A/Int/l | |
| Q9NVA2 | F13gh3 | hypothetical protein | 374.1496 | 861 | 3471.789 | 1568.846 | 3.21E+00 | 1.20E+00 | 9 | Increasing | |
| | | FLJ10849 | | | | | | | | A/Int/l | |
| P40261 | M09cd1 | nicotinamide N- | 1060.579 | 2310 | 5683.533 | 3016.993 | 2.42E+00 | 1.12E+00 | 9 | Increasing | |
| | | methyltransferas | | | | | | | | A/Int/l | |
| Q15063 | C01ef7 | osteobtast specific | 276.834 | 795 | 6325.222 | 3133.301 | 4.91E+00 | 1.52E+00 | 9 | Increasing | |
| | | factor 2 (fasci | | | | | | | | A/Int/l | |
| P09486 | A09ef7 | secreted protein: | 404.2839 | 2760 | 63837.2 | 22333.46 | 7.30E+00 | 2.77E+00 | 9 | Increasing | |
| | | acidic: cysteine | | | | | | | | A/Int/l | |
| P35625 | K20ef5 | tissue inhibitor of | 216.254 | 619 | 10078.17 | 3837.823 | 5.54E+00 | 1.52e+00 | 9 | Increasing | |
| | | metalloproteina | | | | | | | | A/Int/l | |
| Q9H2L5 | G06gh8 | AD037 protein | 361.2297 | 681 | 158.8121 | 400.4602 | 1.19E+00 | 9.15E−01 | 19 | Int> (A:I) | |
| P15121 | C18ab2 | aldo-keto reductase | 2342.744 | 5380 | 2337.052 | 3354.494 | −3.51E−03 | 1.20E+00 | 19 | Int> (A:I) | |
| | | family 1: me | | | | | | | | | |
| P08582 | B14ef5 | antigen p97 | 299.1375 | 595 | 80.80392 | 324.9781 | −1.89E+00 | 9.92E−01 | 19 | Int> (A:I) | |
| | | (melanoma associat | | | | | | | | | |
| P20292 | E06ab2 | arachidonate 5- | 100.0016 | 687 | 203.2911 | 330.0345 | 1.02E+00 | 2.78E+00 | 19 | Int> (A:I) | |
| | | lipoxygenase-act | | | | | | | | | |
| O15342 | A13ab3 | ATPase: H+ | 6063.171 | 16500 | 6160.831 | 9572.914 | 2.31E−02 | 1.44E+00 | 19 | Int> (A:I) | |
| | | transporting; lysoso | | | | | | | | | |
| Q9BXJ0 | H18gh6 | C1q and tumor | 180.2976 | 350 | 154.1441 | 228.0109 | −2.26E−01 | 9.556−01 | 19 | Int> (A:I) | |
| | | necrosis factor re | | | | | | | | | |
| Q9NRJ3 | P09gh4 | CC chemokine CCL28 | 250.0021 | 1830 | 763.2866 | 948.0274 | 1.61E+00 | 2.87E+00 | 19 | Int> (A:I) | |
| P05305 | F17ab5 | endothelin 1 | 866.4561 | 1810 | 403.431 | 1026.992 | −1.10E+00 | 1.06E+00 | 19 | Int> (A:I) | |
| P09341 | A04ab6 | GRO1 oncogene | 933.2938 | 4770 | 1006.711 | 2236.622 | 1.09E−01 | 2.35E+00 | 19 | Int> (A:I) | |
| | | (melanoma gro | | | | | | | | | |
| Q92730 | O14ef4 | GTP-binding protein | 653.8575 | 2760 | 542.7528 | 1318.381 | −2.69E−01 | 2.08E+00 | 19 | Int> (A:I) | |
| Q9H5K0 | L10gh5 | hypothetical protein | 151.2029 | 296 | 130.3196 | 192.6442 | −2.14E−01 | 9.71E−01 | 19 | Int> (A:I) | |
| | | FLJ23360 | | | | | | | | | |
| P18564 | E21ef7 | integrin:beta 6 | 540.0362 | 1180 | 233.0055 | 650.9277 | −1.21E+00 | 1.13E+00 | 19 | Int> (A:I) | |
| P14316 | B16ef6 | interferon regulatory | 252.3603 | 472 | 255.7047 | 326.8164 | 1.90E−02 | 9.04E−01 | 19 | Int> (A:I) | |
| | | factor 2 | | | | | | | | | |
| P40305 | O20ab7 | interferon: alpha- | 117.3152 | 766 | 218.2471 | 367.2688 | 8.96E−01 | 2.71E+00 | 19 | Int> (A:I) | |
| | | inducible protei | | | | | | | | | |
| P05161 | B21cd5 | interferon-stimulated | 302.3796 | 983 | 326.6346 | 537.4035 | 1.11E−01 | 1.70E+00 | 19 | Int> (A:I) | |
| | | protein: 15 | | | | | | | | | |
| P42701 | A06ef7 | interleukin 12 | 1227.347 | 22100 | 1333.521 | 8218.994 | 1.20E−01 | 4.17E+00 | 19 | Int> (A;I) | |
| | | receptor; beta 1 | | | | | | | | | |
| P09237 | J15ef1 | matrix metallo- | 9598.545 | 87300 | 19849.69 | 38905.32 | 1.05E+00 | 3.18E+00 | 19 | Int> (A;I) | |
| | | proteinase 7 (matri | | | | | | | | | |
| P16860 | O13ab8 | natriuretic peptide | 4340.214 | 12900 | 1260.906 | 6171.6 | −1.78E+00 | 1.57E+00 | 19 | Int> (A;I) | |
| | | precursor B | | | | | | | | | |
| P21359 | L16ab7 | neurofibromin 1 | 132.463 | 292 | 155.0822 | 193.3208 | 2.27E−01 | 1.14E+00 | 19 | Int> (A;I) | |
| | | (neurofibromatos | | | | | | | | | |
| P02775 | B07ef7 | pro-platelet basic | 33.98659 | 1400 | 265.2119 | 567.1992 | 2.96E+00 | 5.37E+00 | 19 | Int> (A;I) | |
| | | protein (include | | | | | | | | | |
| Q9UDQ9 | J22gh4 | SBBI26 protein | 121.56661060 236.8624 | 1080 | 238.8624 | 479.5906 | 9.74E−01 | 3.15E+00 | 19 | Int> (A;I) | |
| O75635 | P14cd3 | serine (or cysteine) | 232.6336 | 3890 | 326.2581 | 1483.169 | 4.86E−01 | 4.06E+00 | 19 | Int> (A;I) | |
| | | proteinase in | | | | | | | | | |
| P02735 | A11cd3 | serum amyloid A1 | 496.9578 | 17400 | 957.6141 | 6295.724 | 9.46E−01 | 5.13E+00 | 19 | Int> (A;I) | |
| O60635 | F10cd5 | tetraspan 1 | 321.8888 | 843 | 177.6479 | 447.5729 | −8.58E−01 | 1.39E+00 | 19 | Int> (A;I) | |
| P50591 | C09cd4 | tumor necrosis factor | 536.7773 | 1550 | 476.2305 | 854.3061 | −1.73E−01 | 1.53E+00 | 19 | Int> (A;I) | |
| | | (ligand) su | | | | | | | | | |
| Q03169 | I10ef7 | tumor necrosis factor: | 366.7502 | 1260 | 326.6536 | 650.583 | −1.67E−01 | 1.76E+00 | 19 | Int> (A;I) | |
| | | alpha-indu | | | | | | | | | |
| Q9C075 | N11ef8 | type I intermediate | 848.9829 | 4200 | 401.4854 | 1818.182 | −1.08E+00 | 2.31E+00 | 19 | Int> (A;I) | |
| | | filament cytok | | | | | | | | | |
| P02248 | A01cd3 | ubiquitin C | 155.8735 | 892 | 62.04595 | 369.9913 | −1.33E+00 | 2.52E+00 | 19 | Int> (A:I) | |
| O95497 | A15cd5 | vanin 1 | 309.1556 | 1840 | 510.7208 | 887.0948 | 7.24E−01 | 2.57E+00 | 19 | Int> (A;I) | |
| Q14202 | B15cd5 | zinc finger protein 261 | 199.6246 | 1040 | 247.9161 | 496.3668 | 3.13E−01 | 2.38E+00 | 19 | Int> (A;I) | |
| P15514 | M02ef7 | amphiregulin | 103.1419 | 405 | 65.66067 | 191.2981 | −6.52E−01 | 1.97E+00 | 18 | Off A/I; On Int | |
| | | (schwannoma-deri | | | | | | | | | |
| Q98ZL9 | I21gh8 | B aggressive | 126.3951 | 211 | 84.56927 | 140.6098 | −5.60E−01 | 7.42E−01 | 18 | Off A/I; On Int | |
| | | lymphoma gene | | | | | | | | | |
| P30991 | D14ef5 | chemokine (C-X-C | 64.37115 | 260 | 120.2328 | 148.3131 | 9.01E−01 | 2.02E+00 | 18 | Off A/I; On Int | |
| | | motif); recept | | | | | | | | | |
| P29400 | H13ab3 | collagen; type IV; | 53.50943 | 374 | 141.1609 | 189.6904 | 1.40E+00 | 2.81E+00 | 18 | Off A/I; On Int | |
| | | alpha 5 (Alport | | | | | | | | | |
| Q9UK22 | H22cd7 | F-box only protein 2 | 87.59622 | 217 | 120.3781 | 141.6466 | 4.59E−01 | 1.31E+00 | 18 | Off A/I; On Int | |
| Q16769 | O15cd8 | glutaminyl-peptide | 69.90252 | 195 | 113.0683 | 132.7502 | 3.31E−01 | 1.12E+00 | 18 | Off A/I; On Int | |
| | | cyclotransfera | | | | | | | | | |
| P02261 | M06gh6 | H2A histone family; | 65.46026 | 263 | 126.664 | 151.868 | 9.52E−01 | 2.01E+00 | 18 | Off A/I; On Int | |
| | | member I | | | | | | | | | |
| P20769 | D03ef1 | immunoglobulin heavy | 101.245 | 176 | 102.5667 | 126.7136 | 1.87E−02 | 8.00E−01 | 18 | Off A/I; On Int | |
| | | constant r | | | | | | | | | |
| P52945 | I12ab6 | insulin promoter | 74.60504 | 351 | 66.42558 | 170.7555 | 2.12E−01 | 2.24E+00 | 18 | Off A/I; On Int | |
| | | factor 1; homeo | | | | | | | | | |
| Q14496 | F21cd6 | interferon-induced | 99.27101 | 286 | 98.28057 | 161.3017 | −1.45E−02 | 1.53E+00 | 18 | Off A/I; On Int | |
| | | protein 44 | | | | | | | | | |
| P01583 | F15ef7 | interleukin 1; alpha | 103.386 | 375 | 66.6314 | 181.5566 | −6.34E−01 | 1.86E+00 | 18 | Off A/I; On Int | |
| P01584 | F17ef7 | interleukin 1; beta | 116.7675 | 415 | 119.0156 | 216.8707 | 2.75E−02 | 1.83E+00 | 18 | Off A/I; On Int | |
| Q9GZM1 | I20gh7 | NDRG family | 102.1328 | 348 | 101.569 | 183.8736 | −7.99E−03 | 1.77E+00 | 18 | Off A/I; On Int | |
| | | member 4 | | | | | | | | | |
| P25105 | G15ef5 | platelet-activating | 97.43121 | 356 | 80.47935 | 177.9689 | −2.76E−01 | 1.87E+00 | 18 | Off A/I; On Int | |
| | | factor receptor | | | | | | | | | |
| P58294 | O14gh8 | prokineticin 1 | 79.98286 | 241 | 137.5167 | 152.9437 | 7.82E−01 | 1.59E+00 | 18 | Off A/I; On Int | |
| | | precursor | | | | | | | | | |
| P23471 | A06ef6 | protein tyrosine | 85.55935 | 346 | 93.65845 | 175.0253 | 1.30E−01 | 2.02E+00 | 18 | Off A/I; On Int | |
| | | phosphatase; re | | | | | | | | | |
| O88386 | D10ef3 | RAB10; member RAS | 123.2003 | 399 | 68.13944 | 196.7342 | −8.54E−01 | 1.69E+00 | 18 | Off A/I; On Int | |
| | | oncogene | | | | | | | | | |
| P10826 | I08gh6 | retinoic acid | 67.98871 | 241 | 120.9469 | 143.3844 | 8.31E−Cl | 1.83E+00 | 18 | Off A/I; On Int | |
| | | receptor; beta | | | | | | | | | |
| O95786 | 124ef3 | RNA helicase | 112.9366 | 241 | 89.32396 | 147.8829 | −3.38E−01 | 1.10E+00 | 18 | Off A/I; On Int | |
| P02778 | O10ef7 | small inducible | 65.03395 | 418 | 86.09019 | 189.6022 | 4.05E−01 | 2.68E+00 | 18 | Off A/I; On Int | |
| | | cytokine subfamil | | | | | | | | | |
| P80162 | F14cd1 | small inducible | 54.00821 | 311 | 129.2639 | 164.6594 | 1.26E+00 | 2.52E+00 | 18 | Off A/I; On Int | |
| | | cytokine subfamil | | | | | | | | | |
| P43005 | N23ef5 | solute carrier family | 99.30514 | 189 | 110.8119 | 133.1898 | 1.58E−01 | 9.32E−01 | 18 | Off A/I; On Int | |
| | | 1 (neuronal/ | | | | | | | | | |
| P01375 | O09ef6 | tumor necrosis factor | 138.6153 | 386 | 75.13436 | 200.0804 | −8.84E−01 | 1.48E+00 | 18 | Off A/I; On Int | |
| | | (TNF super | | | | | | | | | |
| Q16890 | G04cd2 | tumor protein D52- | 103.5477 | 191 | 98.01862 | 130.885 | −7.92E−02 | 8.84E−01 | 18 | Off A/I; On Int | |
| | | like 1 | | | | | | | | | |
| Q9H949 | K11gh5 | WW45 protein | 111.8812 | 218 | 79.54574 | 136.5789 | −4.92E−01 | 9.64E−01 | 18 | Off A/I; On Int | |
| Q9H4G4 | O06gh5 | 17kD fetal brain | 88.61746 | 223 | 532.2979 | 281.2148 | 2.59E+00 | 1.33E+00 | 8 | Off Acinar; | |
| | | protein | | | | | | | | Increasing Int/I | |
| Q14040 | B15ef1 | collagen; type VI; | 38.27203 | 209 | 4724.413 | 1657.194 | 6.95E+00 | 2.45E+00 | 8 | Off Acinar; | |
| | | alpha 1 | | | | | | | | Increasing Int/I | |
| P12110 | B17ef1 | collagen; type VI; | 88.00282 | 164 | 3835.637 | 1362.487 | 5.45E+00 | 8.97E−01 | 8 | Off Acinar; | |
| | | alpha 2 | | | | | | | | Increasing Int/I | |
| P02751 | K15ef1 | fibronectin 1 | 4.441362 | 152 | 4171.603 | 1442.812 | 9.88E+00 | 5.10E+00 | 8 | Off Acinar; | |
| | | | | | | | | | | Increasing Int/I | |
| P14652 | N23ab6 | homeo box B2 | 62.71629 | 291 | 3571.974 | 1308.575 | 5.83E+00 | 2.21E+00 | 8 | Off Acinar; | |
| | | | | | | | | | | Increasing Int/I | |
| P08476 | D15ef7 | inhibin; beta A | 64.38161 | 318 | 854.6532 | 412.2794 | 3.73E+00 | 2.30E+00 | 8 | Off Acinar; | |
| | | (activin A; activin | | | | | | | | Increasing Int/I | |
| P17936 | D07ab6 | insulin-like growth | 38.22268 | 349 | 1302.669 | 563.1937 | 5.09E+00 | 3.19E+00 | 8 | Off Acinar; | |
| | | factor binding | | | | | | | | Increasing Int/I | |
| Q9Y4K0 | P18ab6 | lysyl oxidase-like 2 | 75.91549 | 260 | 2525.644 | 953.7036 | 5.06E+00 | 1.77E+00 | 8 | Off Acinar; | |
| | | | | | | | | | | Increasing Int/I | |
| P03956 | N05ef7 | matrix metallo- | 106.2193 | 203 | 590.2622 | 299.8442 | 2.47E+00 | 9.35E−01 | 8 | Off Acinar; | |
| | | proteinase 1 (inter | | | | | | | | Increasing Int/I | |
| P39900 | N15ef7 | matrix matallo- | 18.2367 | 381 | 1219.953 | 539.7471 | 6.06E+00 | 4.39E+00 | 8 | Off Acinar; | |
| | | proteinase 12 (ma | | | | | | | | Increasing Int/I | |
| P55001 | G09ab7 | microfibrillar- | 99.33548 | 526 | 3302.631 | 1309.293 | 5.06E+00 | 2.40E+00 | 8 | Off Acinar; | |
| | | associated protein | | | | | | | | Increasing Int/I | |
| Q9NYR0 | O07gh7 | SH3-domain kinase | 34.69271 | 197 | 656.4675 | 296.0285 | 4.24E+00 | 2.50E+00 | 8 | Off Acinar; | |
| | | binding prot | | | | | | | | Increasing Int/I | |
| O43623 | D06ef4 | snail homolog 2 | 96.88079 | 292 | 3028.508 | 1139.155 | 4.97E+00 | 1.59E+00 | 8 | Off Acinar; | |
| | | (Drosophila) | | | | | | | | Increasing Int/I | |
| P09936 | I13cd4 | ubiquitin carboxyl- | 48.30052 | 219 | 1523.166 | 596.7714 | 4.98E+00 | 2.18E+00 | 8 | Off Acinar; | |
| | | terminal estera | | | | | | | | Increasing Int/I | |
| Q02952 | J11ab2 | A kinase (PRKA) | 103.9756 | 364 | 229.1446 | 232.3779 | 1.14E+00 | 1.81E+00 | 4 | Off Acinar; | |
| | | anchor protein | | | | | | | | On Int = t | |
| Q99541 | I19ab2 | adipose differentiation- | 67.38509 | 182 | 146.1347 | 132.005 | 1.12E+00 | 1.44E+00 | 4 | Off Acinar; | |
| | | related pr | | | | | | | | On Int = t | |
| P54284 | N17ab3 | calcium channel; | 106.3248 | 227 | 254.9633 | 195.985 | 1.26E+00 | 1.09E+00 | 4 | Off Acinar; | |
| | | voltage-depend | | | | | | | | On Int = t | |
| Q13269 | G14ab5 | cAMP responsive | 100.6639 | 193 | 217.7521 | 170.529 | 1.11E+00 | 9.40E−01 | 4 | Off Acinar; | |
| | | element bindin | | | | | | | | On Int = t | |
| Q9NPF2 | M02ef4 | chondroitin | 133.8375 | 192 | 303.01 | 209.7612 | 1.18E+00 | 5.24E−01 | 4 | Off Acinar; | |
| | | 4-sulfotransferase | | | | | | | | On Int = t | |
| Q9H7A5 | F12gh7 | chromosome 20 open | 98.71521 | 179 | 208.0134 | 161.8637 | 1.08E+00 | 8.58E−01 | 4 | Off Acinar; | |
| | | reading fra | | | | | | | | On Int = t | |
| 09H9Q2 | L23gh5 | COP9 constitutive | 86.57682 | 115 | 184.3088 | 128.7341 | 1.09E+00 | 4.14E−01 | 4 | Off Acinar; | |
| | | photomorphog | | | | | | | | On Int = t | |
| P42771 | D17ef5 | cyclin-dependent | 43.35217 | 471 | 355.7426 | 290.1928 | 3.04E+00 | 3.44E+00 | 4 | Off Acinar; | |
| | | kinase inhibitor | | | | | | | | On Int = t | |
| Q14650 | J20ef4 | cytoplasmic FMR1 | 132.5759 | 272 | 321.6348 | 242.2271 | 1.28E+00 | 1.04E+00 | 4 | Off Acinar; | |
| | | interacting pr | | | | | | | | On Int = t | |
| Q96IK6 | F14gh6 | DKFZP434C245 | 145.227 | 104 | 91.06477 | 113.265 | −6.73E−01 | −4.89E−01 | 4 | Off Acinar; | |
| | | protein | | | | | | | | On Int = t | |
| P12034 | D05ef7 | fibroblast growth | 60.2377 | 259 | 317.6018 | 212.2747 | 2.40E+00 | 2.10E+00 | 4 | Off Acinar; | |
| | | factors 5 | | | | | | | | On Int = t | |
| Q93079 | M20gh6 | H2B histone family; | 144.6906 | 428 | 271.2983 | 281.3281 | 9.07E−01 | 1.56E+00 | 4 | Off Acinar; | |
| | | member J | | | | | | | | On Int = t | |
| Q9P016 | A10ef8 | HSPC144 protein | 123.4601 | 348 | 399.4935 | 290.4476 | 1.69E+00 | 1.50E+00 | 4 | Off Acinar; | |
| | | | | | | | | | | On Int = t | |
| Q9HB15 | M09gh4 | HT021 | 116.3605 | 276 | 309.5798 | 233.835 | 1.41E+00 | 1.24E+00 | 4 | Off Acinar; | |
| | | | | | | | | | | On Int = t | |
| Q9BY45 | O24gh8 | HTPAP protein | 78.49388 | 109 | 177.2043 | 121.5622 | 1.17E+00 | 4.74E−01 | 4 | Off Acinar; | |
| | | | | | | | | | | On Int = t | |
| P08397 | G18ab6 | hydroxymethyl- | 139.0857 | 188 | 260.8417 | 196.1361 | 9.07E−01 | 4.38E−01 | 4 | Off Acinar; | |
| | | bilane synthase | | | | | | | | On Int = t | |
| Q9BUV0 | M06gh4 | hypothetical | | 68.40543 | 152 | 167.5592 | 129.3473 | 1.29E+00 | 1.15E+00 | 4 | Off Acinar; |
| | | protein dJ465N24.2. | | | | | | | | On Int = t | |
| Q9H9A2 | N22gh6 | hypothetical protein | 82.54725 | 110 | 169.5379 | 120.7311 | 1.04E+00 | 4.16E−01 | 4 | Off Acinar; | |
| | | DKFZp762L | | | | | | | | On Int = t | |
| Q9NUV6 | P09gh3 | hypothetical protein | 87.43713 | 178 | 136.6771 | 134.1021 | 6.44E−01 | 1.03E+00 | 4 | Off Acinar; | |
| | | FLJ11113 | | | | | | | | On Int = t | |
| Q9NXF7 | C20gh2 | hypothetical protein | 66.49705 | 143 | 170.7367 | 126.7851 | 1.36E+00 | 1.11E+00 | 4 | Off Acinar; | |
| | | FLJ20280 | | | | | | | | On Int = t | |
| Q9H6V0 | M05gh5 | hypothetical protein | 116.3704 | 258 | 323.3964 | 232.5844 | 1.47E+00 | 1.15E+00 | 4 | Off Acinar; | |
| | | FLJ21839 | | | | | | | | On Int = t | |
| Q9BUW5 | K21gh6 | hypothetical protein | 80.31127 | 170 | 203.9516 | 151.3887 | 1.34E+00 | 1.08E+00 | 4 | Off Acinar; | |
| | | MGC4707 | | | | | | | | On Int = t | |
| Q9H765 | I15gh6 | hypothetical protein | 106.3949 | 220 | 206.6405 | 177.6046 | 9.58E−01 | 1.05E+00 | 4 | Off Acinar; | |
| | | MGC5540 | | | | | | | | On Int = t | |
| Q14157 | N23gh1 | KIAA04144 gene | 95.13957 | 147 | 147.7214 | 129.9305 | 6.35E−01 | 6.27E−01 | 4 | Off Acinar; | |
| | | product | | | | | | | | On Int = t | |
| Q9Y2D8 | A07ef8 | KIAA0923 protein | 84.1225 | 237 | 206.8269 | 176.1358 | 1.30E+00 | 1.50E+00 | 4 | Off Acinar; | |
| | | | | | | | | | | On Int = t | |
| Q9H6Z3 | E24gh5 | kinesin family | 111.6382 | 206 | 238.8822 | 185.578 | 1.10E+00 | 8.85E−01 | 4 | Off Acinar; | |
| | | member 13A | | | | | | | | On Int = t | |
| Q99748 | J24ab8 | neurturin | 123.7486 | 150 | 246.4754 | 173.2957 | 9.94E−01 | 2.74E−01 | 4 | Off Acinar; | |
| | | | | | | | | | | On Int = t | |
| Q9NVD7 | D11gh3 | parvin; alpha | 82.67948 | 140 | 230.66 | 151.1753 | 1.48E+00 | 7.62E−01 | 4 | Off Acinar; | |
| | | | | | | | | | | On Int = t | |
| O43175 | H24cd6 | phosphoglycerate | 98.81456 | 119 | 192.3326 | 136.6151 | 9.61E−01 | 2.64E−01 | 4 | Off Acinar; | |
| | | dehydrogenas | | | | | | | | On Int = t | |
| Q9Y253 | E24cd1 | polymerase (DNA | 108.2715 | 101 | 157.8762 | 122.2465 | 5.44E−01 | −1.06E−01 | 4 | Off Acinar; | |
| | | directed); eta | | | | | | | | On Int = t | |
| P24158 | M05ef5 | proteinase 3 | 87.66238 | 280 | 172.8525 | 180.0275 | 9.80E−01 | 1.67E+00 | 4 | Off Acinar; | |
| | | (serine proteinase; | | | | | | | | On Int = t | |
| Q9UBK7 | M21ef3 | RAB; member of RAS | 110.9871 | 189 | 313.4316 | 204.6287 | 1.50E+00 | 7.72E−01 | 4 | Off Acinar; | |
| | | oncogene | | | | | | | | On Int = t | |
| Q13636 | K19cd7 | RAB31: member RAS | 76.36263 | 118 | 170.0352 | 121.5169 | 1.15E+00 | 6.30E−01 | 4 | Off Acinar: | |
| | | oncogene | | | | | | | | On Int = t | |
| Q9UIC2 | L10ef2 | RNB6 | 80.75879 | 171 | 194.395 | 148.7422 | 1.27E+00 | 1.08E+00 | 4 | Off Acinar; | |
| | | | | | | | | | | On lnt = t | |
| Q9NVQ7 | K12gh3 | Sec61 alpha form 2 | 82.6036 | 143 | 162.0674 | 129.1434 | 9.72E−01 | 7.89E−01 | 4 | Off Acinar; | |
| | | | | | | | | | | On Int = t | |
| O75368 | J08cd1 | SH3 domain binding | 92.82513 | 212 | 196.8753 | 167.1823 | 1.08E+00 | 1.19E+00 | 4 | Off Acinar; | |
| | | glutamic aci | | | | | | | | On Int = t | |
| O95863 | N01cd2 | snail homolog 1 | 76.58448 | 134 | 157.9807 | 122.817 | 1.04E+00 | 8.06E−01 | 4 | Off Acinar; | |
| | | (Drosophila) | | | | | | | | On Int = t | |
| Q9UL01 | H01cd8 | squamous cell | 66.70353 | 111 | 176.4557 | 124.5829 | 1.03E+00 | 3.51E−01 | 4 | Off Acinar; | |
| | | carcinoma antigen | | | | | | | | On Int = t | |
| Q16226 | N05cd6 | thioredoxin interacting | 111.1384 | 454 | 529.2322 | 364.6675 | 2.25E+00 | 2.03E+00 | 4 | Off Acinar; | |
| | | protein | | | | | | | | On Int = t | |
| P07996 | A11ef7 | thrombospondin 1 | 79.00174 | 139 | 163.2593 | 127.0819 | 1.05E+00 | 8.15E−01 | 4 | Off Acinar; | |
| | | | | | | | | | | On Int = t | |
| Q99081 | F10ef6 | transcription factor | 112.2294 | 181 | 256.0348 | 182.9347 | 1.19E+00 | 6.86E−01 | 4 | Off Acinar; | |
| | | 12 (HTF4; he | | | | | | | | On Int = t | |
| O95922 | L06ef7 | tubulin tyrosine | 73.12945 | 245 | 253.9213 | 190.8311 | 1.80E+00 | 1.75E+00 | 4 | Off Acinar; | |
| | | ligase-like 1 | | | | | | | | On Int = t | |
| P15036 | H18ef6 | v-ets erythroblastosis | 149.9259 | 114 | 83.55955 | 115.8531 | −8.43E−01 | −3.94E−01 | 4 | Off Acinar; | |
| | | virus E26 | | | | | | | | On Int = t | |
| O95337 | D04ef8 | weakly similar to | 69.55111 | 164 | 284.3593 | 172.7489 | 2.03E+00 | 1.24E+00 | 4 | Off Acinar; | |
| | | glutathione per | | | | | | | | On Int = t | |
| Q9H1B5 | M18gh5 | xylosyltransferase II | 81.87715 | 106 | 174.2539 | 120.5878 | 1.09E+00 | 3.68E−01 | 4 | Off Acinar; | |
| | | | | | | | | | | On Int = t | |
| P55263 | K07ab2 | adenosine kinase | 891.6698 | 720 | 299.6337 | 637.198 | −1.57E+00 | −3.08E−01 | 15 | On A/Int; Down I | |
| P51648 | A22ab2 | aldehyde dehydrogenase | 1955.203 | 1390 | 507.3786 | 1284.046 | −1.95E+00 | −4.93E−01 | 15 | On A/Int; Down I | |
| | | 3 famil | | | | | | | | | |
| P50995 | I08ab2 | annexin A11 | 4888.71 | 3870 | 1773.94 | 3511.843 | −1.46E+00 | −3.36E−01 | 15 | On A/Int; Down I | |
| P12429 | I14ab2 | annexin A3 | 9711.235 | 10800 | 1720.452 | 7424.351 | −2.50E+00 | 1.59E−01 | 15 | On A/Int; Down I | |
| P05026 | L22ab2 | ATPase: Na+/K+ | 3965.733 | 5290 | 1976.367 | 3744.962 | −1.00E+00 | 4.16E−01 | 15 | On A/Int; Down I | |
| | | transporting; be | | | | | | | | | |
| P00751 | M07ab3 | B-factor; properdin | 2984.309 | 2460 | 1178.473 | 2206.568 | −1.34E+00 | −2.81E−01 | 15 | On A/Int; Down I | |
| Q9HA23 | E10gh6 | calponin like | 480.5263 | 528 | 193.0458 | 400.5912 | −1.32E+00 | 1.36E−01 | 15 | On A/Int; Down I | |
| | | transmembrane dor | | | | | | | | | |
| Q16170 | M17ab3 | carcinoembryonic | 984.7628 | 1230 | 212.7712 | 807.5766 | −2.21E+00 | 3.15E−01 | 15 | On A/Int; Down I | |
| | | antigen-related | | | | | | | | | |
| P25774 | P07ab5 | cathepsin S | 857.9751 | 776 | 228.9093 | 620.9386 | −1.91E+00 | −1.45E−01 | 15 | On A/Int; Down I | |
| P21926 | J13ef7 | CD9 antigen (p24) | 2011.966 | 3500 | 833.332 | 2115.74 | −1.27E+00 | 8.00E−01 | 15 | On A/Int; Down I | |
| Q9H2A7 | M19gh5 | chemokine | 650.7961 | 842 | 235.8205 | 576.1228 | −1.46E+00 | 3.71E−01 | 15 | On A/Int; Down I | |
| | | (C-X-C motif) ligand 1 | | | | | | | | | |
| O14493 | G21ab4 | claudin 4 | 1919.128 | 2480 | 799.5104 | 1732.749 | −1.26E+00 | 3.70E−01 | 15 | On A/Int; Down I | |
| P12277 | M06ab4 | creatine kinase; | 373.8119 | 617 | 193.0681 | 394.4895 | −9.53E−01 | 7.22E−01 | 15 | On A/Int; Down I | |
| | | brain | | | | | | | | | |
| Q9NYS7 | E20ef4 | CS box-containing | 596.6891 | 384 | 211.1821 | 397.3278 | −1.50E+00 | −6.35E−01 | 15 | On A/Int; Down I | |
| | | WD protein | | | | | | | | | |
| P53355 | D09ab5 | death-associated | 1325.243 | 1260 | 558.9756 | 1048.818 | −1.25E+00 | −7.03E−02 | 15 | On A/Int; Down I | |
| | | protein kinase | | | | | | | | | |
| O15205 | D17cd6 | diubiquitin | 7363.301 | 14700 | 2196.358 | 8102.423 | −1.75E+00 | 1.00E+00 | 15 | On A/Int; Down I | |
| Q9UFZ2 | C23ef8 | DKFZP564K247 | 1325.797 | 1060 | 313.5237 | 900.9109 | −2.08E+00 | −3.18E−01 | 15 | On A/Int; Down I | |
| | | protein | | | | | | | | | |
| P28582 | M22ab5 | dual specificity | 2077.188 | 1680 | 754.9876 | 1502.864 | −1.46E+00 | −3.09E−01 | 15 | On A/Int; Down I | |
| | | phosphatase 1 | | | | | | | | | |
| Q16690 | B21ab4 | dual specificity | 485.6475 | 305 | 155.0161 | 315.2743 | −1.65E+00 | −6.70E−01 | 15 | On A/Int; Down I | |
| | | phosphatase 5 | | | | | | | | | |
| Q9UJW0 | H10ef1 | dynactin 4 (p62) | 778.5079 | 662 | 335.9831 | 592.1023 | −1.21E+00 | −2.34E−01 | 15 | On A/Int; Down I | |
| | E13gh5 | ectonucleotide | 233.9728 | 378 | 183.3797 | 265.0097 | −3.52E−01 | 6.91E−01 | 15 | On A/Int; Down I | |
| | | pyrophosphatase | | | | | | | | | |
| P00533 | A06ef5 | epidermal growth | 263.2516 | 334 | 182.4044 | 259.7582 | −5.29E−01 | 3.42E−01 | 15 | On A/Int; Down I | |
| | | factor receptor | | | | | | | | | |
| Q13113 | J02cd5 | epithelial protein | 13324.19 | 15500 | 2641.961 | 10494.6 | −2.33E+00 | 2.20E−01 | 15 | On A/Int; Down I | |
| | | up-regutated in | | | | | | | | | |
| P28161 | M09ab6 | glutathione | 1020.341 | 690 | 217.4433 | 642.7437 | −2.23E+00 | −5.63E−01 | 15 | On A/Int; Down I | |
| | | S-transferase M2 (m | | | | | | | | | |
| Q03013 | L06ab3 | glutathione | 333.2767 | 465 | 210.8696 | 336.469 | −6.60E−01 | 4.81E−01 | 15 | On A/Int; Down I | |
| | | S-transferase M4 | | | | | | | | | |
| P52594 | A18ab7 | HIV-1 Rev binding | 439.4544 | 468 | 252.8058 | 386.6697 | −7.98E−01 | 9.00E−02 | 15 | On A/Int; Down I | |
| | | protein | | | | | | | | | |
| Q9NWT9 | D09gh2 | hypothetical protein | 1062.388 | 757 | 368.3558 | 729.2943 | −1.53E+00 | −4.89E−01 | 15 | On A/Int; Down I | |
| | | FLJ20607 | | | | | | | | | |
| Q9BZR4 | L12gh7 | hypothetical protein | 748.0681 | 844 | 160.1449 | 584.0643 | −2.22E+00 | 1.74E−01 | 15 | On A/Int; Down I | |
| | | HT036 | | | | | | | | | |
| Q9BW25 | A19gh6 | hypothetical protein | 315.4695 | 409 | 150.4768 | 291.8079 | −1.07E+00 | 3.76E−01 | 15 | On A/Int; Down I | |
| | | MGC3101 | | | | | | | | | |
| P16144 | I16ab6 | integrin;beta 4 | 490.7243 | 672 | 157.31 | 440.0464 | −1.64E+00 | 4.54E−01 | 15 | On A/Int; Down I | |
| P10145 | O22ef7 | interleukin 8 | 6902.736 | 13300 | 1783.339 | 7330.866 | −1.95E+00 | 9.47E−01 | 15 | On A/Int; Down I | |
| Q05084 | G10ab7 | islet cell autoantigen 1 | 800.5671 | 588 | 209.1329 | 465.8994 | −1.52E+00 | −3.05E−02 | 15 | On A/Int; Down I | |
| | | (69kD) | | | | | | | | | |
| Q9Y484 | E14cd7 | JM5 protein | 1335.218 | 2010 | 938.9827 | 1429.625 | −5.08E−01 | 5.93E−01 | 15 | On A/Int; Down I | |
| P14923 | M23ef6 | junction plakoglobin | 1969.949 | 2610 | 912.3081 | 1831.613 | −1.11E+00 | 4.07E−01 | 15 | On A/Int; Down I | |
| P05783 | N06ef6 | keratin 18 | 17317.24 | 12500 | 2306.177 | 10707.79 | −2.91E+00 | −4.70E−01 | 15 | On A/Int; Down I | |
| P08727 | L06ab6 | keratin 19 | 4025.258 | 2800 | 631.8404 | 2484.93 | −2.67E+00 | −5.25E−01 | 15 | On A/Int; Down I | |
| P08729 | N14ef6 | keratin 7 | 11605.48 | 11100 | 1888.38 | 8203.912 | −2.62E+00 | −6.19E−02 | 15 | On A/Int; Down I | |
| P05787 | N16ef6 | keratin 8 | 11031.01 | 7880 | 1694.024 | 6867.677 | −2.70E+00 | −4.86E−01 | 15 | On A/Int; Down I | |
| O14782 | H22ab6 | kinesin family | 366.3528 | 652 | 242.2822 | 420.177 | −5.97E−01 | 8.31E−01 | 15 | On A/Int; Down I | |
| | | member 3C | | | | | | | | | |
| O00515 | D05ab7 | ladinin 1 | 3562.632 | 2040 | 824.2377 | 2140.683 | −2.11E+00 | −8.08E−01 | 15 | On A/Int; Down I | |
| P80188 | D13ab7 | lipocalin 2 | 5234.435 | 9070 | 664.0466 | 4989.308 | −2.98E+00 | 7.93E−01 | 15 | On A/Int; Down I | |
| | | (oncogene 24p3) | | | | | | | | | |
| Q9UQ53 | G02ef3 | mannosyl (alpha-1; | 1485.548 | 931 | 398.5069 | 938.2331 | −1.90E+00 | −8.75E−01 | 15 | On A/Int; Down I | |
| | | 3-)-glycoprote | | | | | | | | | |
| P80294 | D15gh6 | metallothionein 1H | 327.8489 | 366 | 208.8705 | 300.7815 | −6.50E−01 | 1.57E−01 | 15 | On A/Int; Down I | |
| O75394 | M10cd5 | mitochondrial ribo- | 1441.6574 | 646 | 213.7295 | 433.794 | −1.05E+00 | 5.49E−01 | 15 | On A/Int; Down I | |
| | | somal protein | | | | | | | | | |
| Q9Y376 | H18ef2 | MO25 protein | 860.3225 | 1410 | 550.0258 | 939.6431 | −6.45E−01 | 7.11E−01 | 15 | On A/Int; Down I | |
| P15941 | H22ab7 | mucin 1; trans- | 2330.649 | 1250 | 487.1995 | 1357.116 | −2.26E+00 | −8.95E−01 | 15 | On A/Int; Down I | |
| | | membrane | | | | | | | | | |
| Q16301 | H11cd4 | myelin transcription | 2433.02 | 2560 | 545.0513 | 1844.855 | −2.16E+00 | 7.14E−02 | 15 | On A/Int; Down I | |
| | | factor 2 | | | | | | | | | |
| Q99836 | M07ab7 | myeloid differen- | 1088.39 | 1110 | 413.4864 | 871.4196 | −1.40E+00 | 3.15E−02 | 15 | On A/Int; Down I | |
| | | tiation primary re | | | | | | | | | |
| P24001 | H08cd4 | natural killer cell | 49232.84 | 71300 | 13531.68 | 44684.13 | −1.86E+00 | 5.34E−01 | 15 | On A/Int; Down I | |
| | | transcript 4 | | | | | | | | | |
| Q9C002 | O12gh8 | normal mucosa of | 8605.554 | 17700 | 4423.686 | 10242.28 | −9.60E−01 | 1.04E+00 | 15 | On A/Int; Down I | |
| | | esophagus sp | | | | | | | | | |
| P25963 | A16ef1 | nuclear factor of kappa | 938.908 | 1270 | 605.6118 | 937.0159 | −6.33E−01 | 4.32E−01 | 15 | On A/Int; Down I | |
| | | light poly | | | | | | | | | |
| P00491 | L18ab7 | nucleoside | 820.4015 | 500 | 250.4586 | 523.7383 | −1.71E+00 | −7.13E−01 | 15 | On A/Int; Down I | |
| | | phosphorylase | | | | | | | | | |
| P49763 | O20ef7 | placental growth | 5604.053 | 12600 | 1723.953 | 6846.258 | −1.70E+00 | 1.17E+00 | 15 | On A/Int; Down I | |
| | | factor; vascular | | | | | | | | | |
| Q9Y342 | A13ef2 | plasmolipin | 942.5595 | 983 | 300.2833 | 742.036 | −1.65E+00 | 6.10E−02 | 15 | On A/Int; Down I | |
| P43490 | H04cd5 | pre-B-cell colony- | 2261.72 | 1730 | 651.3251 | 1546.408 | −1.80E+00 | −3.90E−01 | 15 | On A/Int; Down I | |
| | | enhancing fact | | | | | | | | | |
| Q99988 | B23ef7 | prostate | 1470.061 | 2930 | 681.8854 | 1694.813 | −1.11E+00 | 9.96E−01 | 15 | On A/Int; Down I | |
| | | differentiation factor | | | | | | | | | |
| Q9UJY1 | B05ef3 | protein kinase H11 | 1836.927 | 2080 | 544.441 | 1485.828 | −1.75E+00 | 1.77E−01 | 15 | On A/Int; Down I | |
| Q9H1C7 | O10gh8 | putative nuclear | 11454.74 | 14800 | 3062.014 | 9771.26 | −1.90E+00 | 3.69E−01 | 15 | On A/Int; Down I | |
| | | protein ORF1-FI | | | | | | | | | |
| P51149 | A21ef6 | RAB7; member RAS | 3422.13 | 5250 | 2267.107 | 3646.784 | −5.94E−01 | 6.18E−01 | 15 | On A/Int; Down I | |
| | | oncogene f | | | | | | | | | |
| P51151 | J22cd4 | RAB9A; member RAS | 1882.687 | 1580 | 721.6194 | 1393.904 | −1.38E+00 | −2.55E−01 | 15 | On A/Int; Down I | |
| | | oncogene | | | | | | | | | |
| P15153 | C17ef6 | ras-related C3 | 383.0769 | 418 | 213.0301 | 338.0922 | −8.47E−01 | 1.26E−01 | 15 | On A/Int; Down I | |
| | | botulinum toxin s | | | | | | | | | |
| P52566 | I19ef1 | Rho GDP dissociation | 986.1501 | 788 | 352.0789 | 708.6827 | −1,49E+00 | −3.24E−01 | 15 | On A/Int; Down I | |
| | | inhihitor ( | | | | | | | | | |
| P01011 | I08ef7 | serine (or cysteine) | 40450.89 | 43700 | 10172.44 | 31439.89 | −1.99E+00 | 1.11E−01 | 15 | On A/Int; Down I | |
| | | proteinase in | | | | | | | | | |
| P05120 | P07ef7 | serine (or cysteine) | 24922.82 | 38200 | 2879.818 | 21999.69 | −3.11E+00 | 6.16E−01 | 15 | On A/Int; Down I | |
| | | proteinase in | | | | | | | | | |
| O14508 | M13ef6 | STAT induced STAT | 1511.844 | 1490 | 420.463 | 1139.68 | −1.85E+00 | −2.42E−02 | 15 | On A/Int; Down I | |
| | | inhibitor-2 | | | | | | | | | |
| Q9Y6N5 | G13gh4 | sulfide dehydrogenase | 823.7865 | 871 | 284.0637 | 659.5107 | −1.54E+00 | 7.99E−02 | 15 | On A/Int; Down I | |
| | | like (yeas | | | | | | | | | |
| O00161 | C23cd4 | synaptosomal- | 1077.833 | 1340 | 687.0432 | 1034.031 | −6.50E−01 | 3.11E−01 | 15 | On A/Int; Down I | |
| | | associated protein | | | | | | | | | |
| P31431 | F24cd1 | syndecan 4 | 2109.871 | 3080 | 887.6687 | 2026.067 | −1.25E+00 | 5.46E−01 | 15 | On A/Int; Down I | |
| | | (amphiglycan; ryudo | | | | | | | | | |
| O15533 | B16ef7 | TAP binding protein | 313.1585 | 424 | 162.3501 | 299.8834 | −9.48E−01 | 4.38E−01 | 15 | On A/Int; Down I | |
| | | (lapasin) | | | | | | | | | |
| P17987 | E11ef5 | t-complex 1 | 495.6787 | 298 | 157.6175 | 317.0491 | −1.66E+00 | −7.35E−01 | 15 | On A/Int; Down I | |
| O14746 | O20ef6 | telomerase reverse | 264.8687 | 373 | 151.5845 | 262.9869 | −8.05E−01 | 4.92E−01 | 15 | On A/Int; Down I | |
| | | transcriptase | | | | | | | | | |
| P30408 | C06ef1 | transmembrane 4 | 16658.44 | 17400 | 6051.147 | 13372.16 | −1.46E+00 | 6.34E−02 | 15 | On A/Int; Down I | |
| | | superfamily m | | | | | | | | | |
| Q9HCN3 | E07gh5 | transmembrane protein | 947.8492 | 1010 | 462.0955 | 806.2983 | −1.04E+00 | 9.01E−02 | 15 | On A/Int; Down I | |
| | | 8 (five m | | | | | | | | | |
| Q16149 | B15cd1 | transporter 1: ATP- | 662.1561 | 1100 | 460.8685 | 813.3307 | −6.42E−01 | 3.47E−01 | 15 | On A/Int; Down I | |
| | | binding casse | | | | | | | | | |
| Q9Y2A9 | C22ef3 | UDP-GlcNAc:betaGal | 774.5506 | 436 | 153.9485 | 454.5446 | −2.33E+00 | −8.29E−01 | 15 | On A/Int; Down I | |
| | | beta-1;3-N | | | | | | | | | |
| O60625 | N10cd3 | vesicle-associated | 2480.868 | 2680 | 584.39 | 1914.133 | −2.09E+00 | 1.10E−01 | 15 | On A/Int; Down I | |
| | | membrane pr | | | | | | | | | |
| P07948 | A21ab7 | v-yes-1 Yamaguchi | 512.646 | 443 | 170.1742 | 375.1466 | −1.59E+00 | −2.12E−01 | 15 | On A/Int; Down I | |
| | | sarcoma vira | | | | | | | | | |
| Q9P2N4 | M10gh4 | a disintegrin-like | 159.7274 | 227 | 72.96867 | 153.1495 | −1.13E+00 | 5.06E−01 | 14 | On A/Int; Off I | |
| | | and metalloprot | | | | | | | | | |
| P47895 | C04ab2 | aldehyde | 1247.008 | 2370 | 146.6644 | 1254.954 | −3.09E+00 | 9.27E−01 | 14 | On A/Int; Off I | |
| | | dehydrogenase 1 famil | | | | | | | | | |
| P04233 | G09ef1 | CD74 antigen | 229.4566 | 174 | 86.99566 | 163.6002 | −1.40E+00 | −3.98E−01 | 14 | On A/Int; Off I | |
| | | (invariant polypept | | | | | | | | | |
| Q9NZ31 | L12gh4 | chromosome 20 open | 345.4771 | 375 | 137.4415 | 285.9642 | −1.33E+00 | 1.16E−01 | 14 | On A/Int; Off I | |
| | | reading fra | | | | | | | | | |
| O96002 | G07cd5 | chromosome X open | 401.521 | 375 | 130.7316 | 302.4558 | −1.62E+00 | −9.61E−02 | 14 | On A/Int; Off I | |
| | | reading frar | | | | | | | | | |
| O94907 | J15gh6 | dickkopf homolog | 150.3215 | 200 | 84.53571 | 145.044 | −6.30E−01 | 4.14E−01 | 14 | On A/Int; Off I | |
| | | 1 (Xenopus la | | | | | | | | | |
| Q92796 | K06ef5 | discs; large | 161.9581 | 154 | 64.90819 | 133.5164 | −9.32E−01 | −7.56E−02 | 14 | On A/Int; Off I | |
| | | (Drosophila) homolo | | | | | | | | | |
| Q13115 | O17ab4 | duet specificity | 533.5415 | 730 | 146.312 | 469.6392 | −1.87E+00 | 4.52E−01 | 14 | On A/Int; Off I | |
| | | phosphatase 4 | | | | | | | | | |
| P29317 | F02eF5 | EphA2 | 267.722 | 279 | 52.15254 | 209.5143 | −1.70E+00 | 5.76E−02 | 14 | On A/Int; Off I | |
| P29323 | D10eF5 | EphB2 | 269.1596 | 232 | 74.35016 | 191.9946 | −1.66E+00 | −2.11E−01 | 14 | On A/Int; Off I | |
| Q9UKF9 | H10cd7 | ets homologous factor | 603.0262 | 587 | 102.0333 | 430.6372 | −2.56E+00 | −3.92E−02 | 14 | On A/Int; Off I | |
| Q9NPD3 | P22gh2 | exosome component | 273.527 | 300 | 49.72784 | 207.9117 | −2.46E+00 | 1.36E−01 | 14 | On A/Int; Off I | |
| | | Rrp41 | | | | | | | | | |
| O43524 | G12ab4 | forkhead box O3A | 262.2734 | 169 | 102.6435 | 177.9385 | −1.35E+00 | −6.35E−01 | 14 | On A/Int; Off I | |
| P21217 | M16ab3 | fucosyltransferase | 169.3354 | 257 | 60.24803 | 168.6097 | −1.65E+00 | 4.40E−01 | 14 | On A/Int; Off I | |
| | | 3 (galactoside | | | | | | | | | |
| O75205 | D24ef2 | G pretein-coupled | 621.7264 | 382 | 90.93956 | 364.6596 | −2.77E+00 | −7.03E−01 | 14 | On A/Int; Off I | |
| | | receptor; famil | | | | | | | | | |
| O75712 | A03ef1 | gap junction protein; | 235.3139 | 402 | 145.5466 | 280.6655 | −6.93E−01 | 7.72E−01 | 14 | On A/Int; Off I | |
| | | beta 3; 31kD | | | | | | | | | |
| Q92908 | D12ab4 | GATA binding protein | 293.117 | 199 | 93.59786 | 195.3976 | −1.65E+00 | −5.55E−01 | 14 | On A/Int; Off I | |
| | | 6 | | | | | | | | | |
| O95210 | P05cd4 | genethonin 1 | 331.9855 | 241 | 112.69 | 228.6851 | −1.56E+00 | −4.60E−01 | 14 | On A/Int; Off I | |
| O95395 | K21cd5 | glucosaminyl | 297.4236 | 274 | 45.62301 | 205.5863 | −2.70E+00 | −1.21E−01 | 14 | On A/Int; Off I | |
| | | (N-acetyl) transfera | | | | | | | | | |
| P48506 | F22ab5 | glutamate-cysteine | 290.1944 | 243 | 134.9982 | 225.679 | −1.15E+00 | −2.97E−01 | 14 | On A/Int; Off I | |
| | | ligase; cataly | | | | | | | | | |
| Q9UI98 | F08ef8 | hqp0256 protein | 357.567 | 336 | 106.603 | 266.8154 | −1.74E+00 | −8.98E−02 | 14 | On A/Int; Off I | |
| Q9H6D8 | D18gh5 | hypothetical protein | 542.8184 | 595 | 127.4299 | 421.5312 | −2.09E+00 | 1.32E−01 | 14 | On A/Int; Off I | |
| | | FLJ22362 | | | | | | | | | |
| P05362 | I02ab6 | intercellular | 411.0131 | 424 | 87.80016 | 301.0074 | −2.80E+00 | 4.56E−02 | 14 | On A/Int; Off I | |
| | | adhesion molecule 1 | | | | | | | | | |
| Q07627 | J20gh7 | keratin associated | 288.3588 | 303 | 40.26571 | 210.3957 | −2.64E+00 | 8.93E−02 | 14 | On A/Int; Off I | |
| | | protein 1.1 | | | | | | | | | |
| Q96YQ7 | D10gh8 | keratin associated | 493.4934 | 805 | 96.51137 | 396.2261 | −2.35E+00 | 2.93E−01 | 14 | On A/Int; Off I | |
| | | protein 4.10 | | | | | | | | | |
| O75071 | F15gh1 | KIAA0494 gene | 240.2512 | 167 | 91.77931 | 166.3595 | −1.39E+00 | −5.24E−01 | 14 | On A/Int; Off I | |
| | | product | | | | | | | | | |
| Q9UPQ2 | G22ef8 | KIAA1100 protein | 224.7248 | 349 | 135.8611 | 236.4102 | −7.28E−01 | 8.34E−01 | 14 | On A/Int; Off I | |
| O43696 | H10cd6 | kinesin family | 159.6982 | 170 | 73.44924 | 134.4385 | −1.12E+00 | 9.16E−02 | 14 | On A/Int; Off I | |
| | | member 1C | | | | | | | | | |
| Q13887 | L05ab5 | Kruppel-like factor | 494.1259 | 376 | 97.43054 | 322.6178 | −2.34E+00 | −3.93E−01 | 14 | On A/Int; Off I | |
| | | 5 (intestinal) | | | | | | | | | |
| O00312 | D22cd3 | MAP kinase-inter- | 147.9629 | 150 | 86.60695 | 126.2701 | −7.73E−01 | 2.20E−02 | 14 | On A/Int; Off I | |
| | | acting serine/th | | | | | | | | | |
| O15264 | H07ab8 | mitogen-activated | 213.7425 | 160 | 76.32089 | 150.0667 | −1.49E+00 | −4.17E−01 | 14 | On A/Int; Off I | |
| | | protein kinase | | | | | | | | | |
| Q9UHA4 | P10gh1 | mitogen-activated | 268.8101 | 244 | 72.50042 | 195.0133 | −1.89E+00 | −1.41E−01 | 14 | On A/Int; Off I | |
| | | protein kinase | | | | | | | | | |
| P21397 | F23ef6 | monoamine oxidase A | 27.3495 | 480 | 30.42645 | 312.5207 | −3.81E+00 | 1.67E−01 | 14 | On A/Int; Off I | |
| P55196 | N07ab7 | myeloid/lymphoid or | 164.9238 | 168 | 78.62952 | 137.1449 | −1.07E+00 | 2.58E−02 | 14 | On A/Int; Off I | |
| | | mixed-linea | | | | | | | | | |
| Q12965 | I24ab7 | myosin 1E | 212.4935 | 276 | 122.0015 | 203.3855 | −8.01E−01 | 3.75E−01 | 14 | On A/Int; Off I | |
| Q9HBW1 | O10gh7 | NAG14 protein | 417.9637 | 396 | 66.84398 | 300.2834 | −2.27E+00 | −7.79E−02 | 14 | On A/Int; Off I | |
| Q9H2W4 | D20gh6 | neural precursor | 361.8431 | 463 | 75.41197 | 299.9778 | −2.26E+00 | 3.56E−01 | 14 | On A/Int; Off I | |
| | | cell espressed; | | | | | | | | | |
| Q9NRR3 | O06gh4 | non-kinase Cdc42 | 194.5075 | 327 | 117.0936 | 213.1258 | −7.34E−01 | 7.49E−01 | 14 | On A/Int; Off I | |
| | | effector protei | | | | | | | | | |
| P23511 | M19ab8 | nuclear transcription | 239.2485 | 226 | 132.3351 | 199.2783 | −8.54E−01 | −8.08E−02 | 14 | On A/Int; Off I | |
| | | factor Y; alp | | | | | | | | | |
| Q99650 | D14cd4 | oncostatin M receptor | 337.1614 | 233 | 141.0848 | 237.114 | −1.26E+00 | −5.33E−01 | 14 | On A/Int; Off I | |
| P04085 | O16ef7 | platetet-derived | 441.7554 | 463 | 124.4793 | 343.2015 | −1.83E+00 | 6.89E−02 | 14 | On A/Int; Off I | |
| | | growth factor alp | | | | | | | | | |
| Q13048 | N06cd2 | pregnancy specific | 149.7737 | 203 | 84.53571 | 145.8869 | −8.25E−01 | 4.41E−01 | 14 | On A/Int; Off I | |
| | | beta-1-glyco | | | | | | | | | |
| P10586 | E11ef6 | protein tyrosine | 466.2876 | 405 | 138.6718 | 335.6682 | −1.77E+00 | −2.05E−01 | 14 | On A/Int; Off I | |
| | | phosphatase; re | | | | | | | | | |
| O95200 | N22ab8 | retinoic acid | 437.2899 | 315 | 72.62659 | 275.0752 | −2.59E+00 | −4.72E−01 | 14 | On A/Int; Off I | |
| | | receptor responder | | | | | | | | | |
| Q9NVX8 | P17ef3 | Rho GTPase activating | 425.2848 | 479 | 109.2541 | 337.8544 | −1.96E+00 | 1.72E−01 | 14 | On A/Int; Off I | |
| | | protein 8 | | | | | | | | | |
| Q15418 | N14ef5 | ribosomal protein S6 | 299.528 | 361 | 78.38666 | 246.2347 | −1.93E+00 | 2.68E−01 | 14 | On A/Int; Off I | |
| | | kinase; 90k | | | | | | | | | |
| Q15434 | F10ab8 | RNA binding motif; | 454.6438 | 325 | 145.2438 | 309.3377 | −1.65E+00 | −4.70E−01 | 14 | On A/Int; Off I | |
| | | single strand | | | | | | | | | |
| Q15437 | O10cd6 | Sec23 homolog B | 281.2005 | 235 | 126.9582 | 214.4833 | −1.15E+00 | −2.58E−01 | 14 | On A/Int; Off I | |
| | | (S. cerevisiae) | | | | | | | | | |
| O60679 | D15ef5 | serum-inducible kinase | 202.0388 | 194 | 62.20918 | 152.8498 | −1.70E+00 | −5.63E−02 | 14 | On A/Int; Off I | |
| Q12690 | N06ef5 | SFRS protein kinase 1 | 350.4057 | 278 | 113.0428 | 247.1823 | −1.83E+00 | −3.33E−01 | 14 | On A/Int; Off I | |
| Q12971 | L03cd6 | sialyltransferase | 247.3141 | 329 | 98.91002 | 224.3379 | −1.35E+00 | 4.11E−01 | 14 | On A/Int; Off I | |
| P78556 | M02cd2 | small inducible | 308.0437 | 352 | 59.31325 | 249.7105 | −2.38E+00 | 3.10E−01 | 14 | On A/Int; Off I | |
| | | cytokine subfamil | | | | | | | | | |
| O75751 | F14ef4 | solute carrier family | 164.6419 | 238 | 86.83593 | 156.4431 | −1.30E+00 | 5.31E−01 | 14 | On A/Int; Off I | |
| | | 22 (extraneu | | | | | | | | | |
| Q9UM01 | D08cd4 | solute carrier family | 201.657 | 280 | 55.69528 | 172.4811 | −1.85E+00 | 3.86E−01 | 14 | On A/Int; Off I | |
| | | 7 (cationic a | | | | | | | | | |
| Q9Y5X1 | D10ef2 | sorting nexin 9 | 219.1839 | 240 | 108.9101 | 189.2961 | −1.01E+00 | 1.30E−01 | 14 | On A/Int; Off I | |
| P08842 | D19ab3 | steroid sulfatase | 436.4255 | 414 | 136.2997 | 328.904 | −1.88E+00 | −7.62E−02 | 14 | On A/Int; Off I | |
| | | (microsomal: a | | | | | | | | | |
| O43760 | G09cd5 | synaptogyrin 2 | 391.0545 | 238 | 118.1187 | 249.1868 | −1.73E+00 | −7.14E−01 | 14 | On A/Int; Off I | |
| O75674 | N15cd5 | target of myb1-like | 278.5234 | 265 | 54.32353 | 205.2456 | −2.35E+00 | 4.30E−02 | 14 | On A/Int; Off I | |
| | | 1 (chicken) | | | | | | | | | |
| P01135 | K04ef7 | transforming growth | 215.9023 | 180 | 59.83082 | 145.3108 | −1.86E+00 | −4.29E−01 | 14 | On A/Int; Off I | |
| | | factor: alpha | | | | | | | | | |
| Q9NT70 | K13gh3 | transmembrane protein | 385.9385 | 401 | 129.2807 | 298.7991 | −1.50E+00 | 1.33E−01 | 14 | On A/Int; Off I | |
| | | vezatin | | | | | | | | | |
| O60656 | G13gh3 | UDP glycosyl- | 548.3937 | 438 | 27.51254 | 337.3961 | −4.31E+00 | −3.18E−01 | 14 | On A/Int; Off I | |
| | | transferase 1 family | | | | | | | | | |
| Q9NZ42 | N18gh4 | uncharacterized | 196.9377 | 213 | 115.2737 | 175.1842 | −7.73E−01 | 1.15E−01 | 14 | On A/Int; Off I | |
| | | hematopoietic s | | | | | | | | | |
| O76080 | E14cd4 | zinc finger protein | 314.9706 | 211 | 74.56541 | 200.0768 | −2.08E+00 | −5.80E−01 | 14 | On A/Int; Off I | |
| | | 216 | | | | | | | | | |
| Q13015 | H11ab2 | ALL 1-fused gene | 329.6081 | 1730 | 1752.9 | 1272.138 | 2.41E+00 | 2.40E+00 | 6 | On Acinar; | |
| | | from chromoso | | | | | | | | Up Int = I | |
| P04083 | I06ab2 | annexin A1 | 1114.486 | 5240 | 8292.245 | 5883.787 | 2.90E+00 | 2.89E+00 | 6 | On Aciner; | |
| | | | | | | | | | | Up Int = I | |
| O60592 | B14ab2 | Arg/Abl-interacting | 1576.114 | 3320 | 2017.536 | 2304.427 | 3.56E−01 | 1.07E+00 | 6 | On Acinar; | |
| | | protein Arg8 | | | | | | | | Up Int = I | |
| Q07814 | E04ef6 | BCL2-associated X | 430.8903 | 862 | 1101.457 | 797.9572 | 1.35E+00 | 1.00E+00 | 6 | On Acinar; | |
| | | protein | | | | | | | | Up Int = I | |
| Q14201 | E17cd7 | BTG family; | 197.8608 | 451 | 419.4375 | 355.9498 | 1.08E+00 | 1.19E+00 | 6 | On Acinar; | |
| | | member 3 | | | | | | | | Up Int = I | |
| Q9NR00 | J21gh4 | chromosome 8 open | 2766.931 | 7310 | 3283.111 | 4453.955 | 2.47E−01 | 1.40E+00 | 6 | On Acinar; | |
| | | reading fra | | | | | | | | Up Int = I | |
| Q96UW7 | K19gh6 | chromosome 9 open | 228.4878 | 650 | 551.4272 | 476.5934 | 1.27E+00 | 1.51E+00 | 6 | On Acinar; | |
| | | reading fra | | | | | | | | Up Int = I | |
| O95401 | N04cd4 | cofactor required | 156.3494 | 395 | 588.6133 | 390.063 | 1.86E+00 | 1.06E+00 | 6 | On Acinar; | |
| | | for Sp1 transcri | | | | | | | | Up Int = I | |
| Q00535 | B09ef5 | cyclin-dependent | 269.7495 | 476 | 432.1176 | 392.829 | 6.80E−01 | 8.19E−01 | 6 | On Acinar; | |
| | | kinase 5 | | | | | | | | Up Int = I | |
| P21741 | E17ab7 | midkine (neurite | 2991.463 | 15400 | 9802.858 | 9388.855 | 1.71E+00 | 2.36E+00 | 6 | On Acinar; | |
| | | growth-promotin | | | | | | | | Up Int = I | |
| Q9Y291 | J15ef2 | mitochondrial | 177.4689 | 330 | 365.4484 | 290.8529 | 1.04E+00 | 8.93E−01 | 6 | On Acinar; | |
| | | ribosomal protein S | | | | | | | | Up Int = I | |
| O75376 | E17cd8 | nuclear receptor | 375.3879 | 1270 | 727.3836 | 791.9351 | 9.54E−01 | 1.78E+00 | 6 | On Acinar; | |
| | | co-repressor 1 | | | | | | | | Up Int = I | |
| P03973 | N10cd1 | secretory leukocyte | 592.855 | 2070 | 4153.51 | 2270.718 | 2.81E+00 | 1.80E+00 | 6 | On Acinar; | |
| | | protease inhi | | | | | | | | Up Int = I | |
| O15427 | F06cd4 | solute carrier | 228.58 | 753 | 785.8975 | 559.0175 | 1.78E+00 | 1.72E+00 | 6 | On Aciner; | |
| | | family 16 (monocar | | | | | | | | Up Int = I | |
| P16949 | D11ab7 | stathmin 1/ | 728.2643 | 2830 | 1984.807 | 1761.654 | 1.45E+00 | 1.85E+00 | 6 | On Acinar; | |
| | | oncoprotein 18 | | | | | | | | Up Int = I | |
| Q9NZ86 | J08gh3 | uncharacterized | 311.3754 | 945 | 536.938 | 597.7198 | 7.66E−01 | 1.60E+00 | 6 | On Acinar; | |
| | | bone marrow pr | | | | | | | | Up Int = I | |
| O00748 | H07cd4 | carboxylesterase 2 | 1080.553 | 220 | 253.3823 | 517.9952 | −2.09E+00 | −2.30E+00 | 20 | Other | |
| | | (intestine; liv | | | | | | | | | |
| Q13630 | M01cd3 | tissue specific | 622.3905 | 287 | 255.576 | 386.1959 | −1.25E+00 | −1.12E+00 | 20 | Other | |
| | | transplantation an | | | | | | | | | |
| P00505 | C14ab6 | glutamic-oxaloacetic | 494.0876 | 356 | 280.2283 | 389.968 | −9.25E−01 | −4.75E−01 | 20 | Other | |
| | | transaminas | | | | | | | | | |
| P50440 | A09ab5 | glycine amidino- | 1737.906 | 395 | 261.2318 | 798.1958 | −2.73E+00 | −2.14E+00 | 20 | Other | |
| | | transferase (L-ar | | | | | | | | | |
| P29622 | A06cd1 | serine (or cysteine) | 461.3518 | 189 | 264.9574 | 304.9848 | 8.00E−01 | −1.29E+00 | 20 | Other | |
| | | proteinase in | | | | | | | | | |
| O14646 | C17ab4 | chromodomain helicase | 867.7631 | 150 | 273.1703 | 440.3418 | −1.67E+00 | −2.27E+00 | 20 | Other | |
| | | DNA bin | | | | | | | | | |
| P48307 | I10cd4 | tissue factor pathway | 920.3007 | 366 | 277.6619 | 521.385 | −1.73E+00 | −1.33E+00 | 20 | Other | |
| | | inhibitor 2 | | | | | | | | | |
| Q15125 | D08cd6 | emopamil binding | 897.4306 | 230 | 282.4317 | 470.0852 | −1.679+00 | −1.96E+00 | 20 | Other | |
| | | protein (sterol | | | | | | | | | |
| P19387 | B13ab8 | polymerase (RNA) II | 196.4282 | 155 | 282.8549 | 211.5128 | 5.26E−01 | −3.39E−01 | 20 | Other | |
| | | (DNA direct | | | | | | | | | |
| Q9P0K7 | H08gh6 | retinoic acid induced | 177.963 | 275 | 283.8007 | 245.4643 | 6.73E−01 | 6.26E−01 | 20 | Other | |
| | | 14 | | | | | | | | | |
| P30086 | E04ab8 | prostatic binding | 634.2686 | 462 | 300.6211 | 465.627 | −1.08E+00 | −4.57E−01 | 20 | Other | |
| | | protein | | | | | | | | | |
| Q13045 | C06ab5 | flightless I homolog | 622.7072 | 501 | 306.0777 | 476.5407 | −1.02E+00 | −3.14E−01 | 20 | Other | |
| | | (Drosophila) | | | | | | | | | |
| O95084 | G10cd7 | protease; serine; 23 | 1096.756 | 246 | 314.746 | 552.6166 | −1.80E+00 | −2.15E+00 | 20 | Other | |
| P49770 | L11ef4 | eukaryotic translation | 731.6826 | 547 | 329.7361 | 536.2071 | −1.15E+00 | −4.19E−01 | 20 | Other | |
| | | initiation fa | | | | | | | | | |
| Q13948 | J02ef6 | cut-like 1; CCAAT | 222.2758 | 349 | 330.5149 | 300.5112 | 5.72E−01 | 6.50E−01 | 20 | Other | |
| | | displacement | | | | | | | | | |
| Q30201 | H05ab3 | hemochromatosis | 205.1607 | 291 | 339.4607 | 278.446 | 7.28E−01 | 5.03E−01 | 20 | Other | |
| P52895 | I16gh6 | aldo-keto reductase | 4001.572 | 263 | 339.7988 | 1534.634 | −3.56E+00 | −3.93E+00 | 20 | Other | |
| | | family 1; me | | | | | | | | | |
| Q9Y5N1 | M24cd7 | histamine receptor H3 | 526.5133 | 203 | 341.1301 | 356.7517 | −6.26E−01 | −1.38E+00 | 20 | Other | |
| P43378 | N23ab8 | protein tyrosine | 1360.729 | 445 | 368.4734 | 724.8175 | −1.88E+00 | −1.61E+00 | 20 | Other | |
| | | phosphatase; no | | | | | | | | | |
| P35414 | O11ab2 | angiotensin | 620.7039 | 406 | 395.8948 | 474.2345 | −6.49E−01 | −6.12E−01 | 20 | Other | |
| | | receptor-like 1 | | | | | | | | | |
| Q14802 | K03cd1 | FXYD domain- | 2174.215 | 711 | 402.87 | 1096.127 | −2.43E+00 | −1.61E+00 | 20 | Other | |
| | | containing ion tra | | | | | | | | | |
| Q9Y266 | F16cd6 | nuclear distribution | 745.3985 | 447 | 408.3037 | 533.6949 | −8.68E−01 | −7.37E−01 | 20 | Other | |
| | | gene C hom | | | | | | | | | |
| P30043 | O21ab3 | biliverdin reductase | 877.8813 | 565 | 417.1383 | 620.0433 | −1.07E+00 | −6.35E−01 | 20 | Other | |
| | | B (flavin red | | | | | | | | | |
| Q9NP73 | L06gh3 | uncharacterized | 804.2103 | 446 | 421.5183 | 557.2055 | −9.32E−01 | −8.51E−01 | 20 | Other | |
| | | hematopoietic s | | | | | | | | | |
| Q12972 | D17ab8 | protein phosphatase | 251.1796 | 358 | 425.52 | 345.0523 | 7.61E−01 | 5.13E−01 | 20 | Other | |
| | | 1; regulator | | | | | | | | | |
| Q9Y296 | N21ef2 | PTD009 protein | 738.5086 | 424 | 436.6114 | 532.9995 | −7.58E−01 | −8.01E−01 | 20 | Other | |
| Q02750 | K02eF7 | mitogen-activated | 810.206 | 508 | 441.4249 | 586.6635 | −8.76E−01 | −6.72E−01 | 20 | Other | |
| | | protein kinase | | | | | | | | | |
| P42226 | G23cd2 | signal transducer and | 692.6025 | 516 | 450.4229 | 619.5568 | −9.87E−01 | −7.92E−01 | 20 | Other | |
| | | activator o | | | | | | | | | |
| P31947 | M07ef6 | stratifin | 2941.197 | 741 | 452.267 | 1378.205 | −2.70E+00 | −1.99E+00 | 20 | Other | |
| P46597 | J12ab2 | acetylserotonin | 2225.944 | 437 | 466.9229 | 1043.272 | −2.25E+00 | −2.35E+00 | 20 | Other | |
| | | O-methyltransfe | | | | | | | | | |
| O14745 | J24cd4 | solute carrier family | 1844.719 | 642 | 504.4269 | 997.106 | −1.87E+00 | −1.52E+00 | 20 | Other | |
| | | 9 (sodium/hy | | | | | | | | | |
| Q9NS67 | D24gh1 | G protein-coupled | 1464.475 | 381 | 595.0192 | 813.4043 | −1.30E+00 | −1.94E+00 | 20 | Other | |
| | | receptor 27 | | | | | | | | | |
| Q9UKL5 | J07cd8 | programmed cell death | 995.6375 | 886 | 605.9272 | 829.3129 | −7.16E−01 | −1.68E−01 | 20 | Other | |
| | | 6 interac | | | | | | | | | |
| Q9UQL3 | B11ef2 | liver-specific | 825.7324 | 1150 | 606.7901 | 860.8974 | −4.44E−01 | 4.78E−01 | 20 | Other | |
| | | bHLH-Zip transcript | | | | | | | | | |
| O9NZZ1 | P18ef2 | hypothetical protein | 397.5554 | 450 | 618.4035 | 488.5944 | 6.37E−01 | 1.78E−01 | 20 | Other | |
| | | HSPC138 | | | | | | | | | |
| Q9Y3Q3 | H03cd7 | integral type I | 526.2023 | 355 | 631.4284 | 504.2124 | 2.63E−01 | −5.68E−01 | 20 | Other | |
| | | protein | | | | | | | | | |
| Q9NSN3 | C10gh4 | similar to aspartate | 355.1305 | 273 | 667.4052 | 431.8528 | 9.10E−01 | −3.79E−01 | 20 | Other | |
| | | beta hydroxy | | | | | | | | | |
| Q9Y5Z0 | B12gh7 | beta-site APP-cleaving | 1489.687 | 960 | 680.3343 | 1036.811 | −1.11E+00 | −6.14E−01 | 20 | Other | |
| | | enzyme 2 | | | | | | | | | |
| | C21gh7 | H2B histone family; | 511.559 | 855 | 733.4035 | 699.9713 | 5.20E−01 | 7.41E−01 | 20 | Other | |
| | | member B | | | | | | | | | |
| O14713 | M11cd5 | integrin cytoplasmic | 446.3806 | 746 | 737.9643 | 643.5596 | 7.25E−01 | 7.42E−01 | 20 | Other | |
| | | domain-ass | | | | | | | | | |
| P46095 | P11ab4 | G protein-coupled | 2336.841 | 460 | 760.4217 | 1185.918 | −1.62E+00 | −2.34E+00 | 20 | Other | |
| | | receptor 6 | | | | | | | | | |
| Q9BV10 | K07gh6 | hypothetical protein | 1761.827 | 653 | 779.7338 | 1064.815 | −1.18E+00 | −1.43E+00 | 20 | Other | |
| | | MGC3136 | | | | | | | | | |
| Q99653 | O08cd7 | calcium binding | 1713.549 | 1430 | 793.2383 | 1313.03 | −1.11E+00 | −2.59E−01 | 20 | Other | |
| | | protein P22 | | | | | | | | | |
| P41584 | P16gh1 | polymerase (RNA) | 560.1066 | 892 | 821.9856 | 758.0427 | 5.53E−01 | 6.71E−01 | 20 | Other | |
| | | II (DNA direct | | | | | | | | | |
| P11940 | E06ab8 | poly(A) binding | 1738.585 | 669 | 827.8343 | 1078.525 | −1.07E+00 | −1.38E+00 | 20 | Other | |
| | | protein: cytoplas | | | | | | | | | |
| Q00341 | G21ab6 | high density | 545.5844 | 444 | 866.5328 | 618.5713 | 6.67E−01 | −2.99E−01 | 20 | Other | |
| | | lipoprotein binding | | | | | | | | | |
| Q16795 | K08ab7 | NADH dehydrogenase | 1582.872 | 1190 | 873.0478 | 1214.409 | −8.58E−01 | −4.15E−01 | 20 | Other | |
| | | (ubiquino | | | | | | | | | |
| Q13082 | E05ab7 | microtubule-associated | 1217.396 | 472 | 873.8683 | 854.4272 | −4.78E−01 | −1.37E+00 | 20 | Other | |
| | | protein 4 | | | | | | | | | |
| P36405 | D16ab2 | ADP-ribosylation | 508.3031 | 547 | 939.7284 | 665.1099 | 8.87E−01 | 1.07E−01 | 20 | Other | |
| | | factor-like 3 | | | | | | | | | |
| Q9NXH2 | C04gh2 | hypothetical protein | 515.6731 | 467 | 959.9781 | 647.4269 | 8.97E−01 | −1.44E−01 | 20 | Other | |
| | | FLJ20254 | | | | | | | | | |
| Q9Y323 | D21ef2 | hypothetical protein | 838.9663 | 1630 | 976.2242 | 1148.545 | 2.19E−01 | 9.59E−01 | 20 | Other | |
| | | HSPC177 | | | | | | | | | |
| P48059 | I12ab7 | LIM and senescent cell | 560.9674 | 918 | 1003.945 | 827.7691 | 8.40E−01 | 7.11E−01 | 20 | Other | |
| | | antigen-li | | | | | | | | | |
| Q9P0M9 | M02ef2 | mitochondrial | 579.0083 | 936 | 1024.359 | 846.5801 | 8.23E−01 | 6.93E−01 | 20 | Other | |
| | | ribosomal protein | | | | | | | | | |
| Q13813 | E07cd2 | spectrin; alpha; | 2261.592 | 886 | 1070.984 | 1406.107 | −1.08E+00 | −1.35E+00 | 20 | Other | |
| | | non-erythrocytic | | | | | | | | | |
| Q9Y379 | H07ef2 | CGI-69 protein | 2556.713 | 1900 | 1174.107 | 1877.939 | −1.12E+00 | −4.26E−01 | 20 | Other | |
| Q13247 | O13gh1 | splicing factor: | 718.1614 | 1210 | 1194.951 | 1041.827 | 7.35E−01 | 7.55E−01 | 20 | Other | |
| | | arginine/serine-rich | | | | | | | | | |
| P14416 | D03ef6 | dopamine receptor D2 | 614.5351 | 745 | 1299.03 | 952.9439 | 6.73E−01 | −1.28E−01 | 20 | Other | |
| Q06323 | A15ef7 | proteasome (prosome; | 1491.803 | 2350 | 1386.719 | 1742.377 | −1.05E−01 | 6.55E−01 | 20 | Other | |
| | | macropai | | | | | | | | | |
| Q9NR31 | H17gh4 | SAR1 protein | 752.6343 | 886 | 1386.849 | 1008.646 | 8.82E−01 | 2.36E−01 | 20 | Other | |
| P05451 | C07cd3 | regenerating islet- | 75644.12 | 1690 | 1394.425 | 26243.72 | −5.76E+00 | −5.45E+00 | 20 | Other | |
| | | derived 1 alph | | | | | | | | | |
| P04075 | C12ab2 | aldolase A; fructose- | 4534.199 | 1590 | 1470.967 | 2532.66 | −1.62E+00 | −1.51E+00 | 20 | Other | |
| | | bisphosphat | | | | | | | | | |
| Q15904 | A19ah3 | ATPase: H+ | 833.0193 | 1380 | 1471.799 | 1228.765 | 8.21E−01 | 7.30E−01 | 20 | Other | |
| | | transporting; lysoso | | | | | | | | | |
| P15559 | G24et7 | NAD(P)H dehydro- | 537.0892 | 303 | 1486.45 | 775.5735 | 1.47E+00 | −8.25E−01 | 20 | Other | |
| | | genase: quino | | | | | | | | | |
| O00303 | L20cd3 | eukaryotic translation | 1243.805 | 636 | 1556.448 | 1145.377 | 3.23E−01 | −9.68E−01 | 20 | Other | |
| | | initiation fa | | | | | | | | | |
| P56705 | M01gh7 | wingless-type MMTV | 3589.752 | 1590 | 1635.805 | 2271.364 | −1.13E+00 | −1.18E+00 | 20 | Other | |
| | | integration | | | | | | | | | |
| P39210 | I04ef7 | MpV17 transgene: | 990.336 | 894 | 1723.06 | 1202.587 | 7.99E−01 | −1.47E−01 | 20 | Other | |
| | | murine homol | | | | | | | | | |
| Q9UKL9 | N03ef4 | aldo-keto reductase | 16311.31 | 2840 | 1896.102 | 7016.796 | −3.10E+00 | −2.52E+00 | 20 | Other | |
| | | family 1; me | | | | | | | | | |
| O43516 | E12cd3 | Wiskolt-Aldrich | 1162.511 | 1190 | 1979.246 | 1444.29 | 7.68E−01 | 3.51E−02 | 20 | Other | |
| | | syndrome protei | | | | | | | | | |
| Q9Y470 | D01ef7 | stem cell growth | 1346.693 | 1110 | 2049.542 | 1503.556 | 6.06E−01 | −2.73E−01 | 20 | Other | |
| | | factor: lymphocy | | | | | | | | | |
| P29312 | H12cd4 | tyrosine 3-mono- | 5187.766 | 4710 | 2331.854 | 4076.247 | −1.15E+00 | −1.40E−01 | 20 | Other | |
| | | oxygenase/trypt | | | | | | | | | |
| Q9Y5R8 | P24gh4 | MUM2 protein | 1596.032 | 1750 | 2568.113 | 1971.095 | 6.86E−01 | 1.32E−01 | 20 | Other | |
| P17676 | D01ab5 | CCAAT/enhancer | 6064.068 | 2880 | 2723.688 | 3890.676 | −1.15E+00 | −1.07E+00 | 20 | Other | |
| | | binding protei | | | | | | | | | |
| O95362 | F11gh6 | differentially | 10407.52 | 2680 | 2912.158 | 5333.102 | −1.84E+00 | −1.96E+00 | 20 | Other | |
| | | expressed in hemal | | | | | | | | | |
| O60417 | C11gh7 | S100 calcium binding | 6955.642 | 5690 | 3097.579 | 5248.939 | −1.17E+00 | −2.89E−01 | 20 | Other | |
| | | protein A1 | | | | | | | | | |
| P19623 | E17cd2 | spermidine synthase | 1644.266 | 1020 | 3417.978 | 2027.643 | 1.06E+00 | −6.88E−01 | 20 | Other | |
| Q9P039 | P02ef7 | FXYD domain- | 2410.08 | 2120 | 4486.029 | 3004.627 | 8.96E−01 | −1.87E−01 | 20 | Other | |
| | | containing ion tra | | | | | | | | | |
| Q9BUF5 | F05gh8 | tubulin beta-5 | 2252.623 | 1580 | 4730.755 | 2855.744 | 1.07E+00 | −5.08E−01 | 20 | Other | |
| P00995 | C23cd2 | serine protease | 67989.21 | 11300 | 5416.175 | 28226.58 | −3.65E+00 | −2.59E+00 | 20 | Other | |
| | | inhibitor; Kazal | | | | | | | | | |
| P11142 | E22ef7 | heat shock 70kD | 11154.99 | 7840 | 5433.843 | 8144.601 | −1.04E+00 | −5.08E−01 | 20 | Other | |
| | | protein 8 | | | | | | | | | |
| P10620 | E16ef7 | microsomal glutathione | 6706.736 | 2430 | 5571.411 | 4901.595 | −2.68E−01 | −1.47E+00 | 20 | Other | |
| | | S-transfe | | | | | | | | | |
| Q13347 | N02cd3 | eukaryotic translation | 19633.03 | 5210 | 5660.757 | 10168.91 | −1.79E+00 | −1.91E+00 | 20 | Other | |
| | | initiation fa | | | | | | | | | |
| P15880 | B16cd1 | ribosomal protein S2 | 4356.01 | 2060 | 5964.993 | 4127.437 | 4.54E−01 | −1.08E+00 | 20 | Other | |
| Q9HCU0 | E24gh4 | tumor endothelial | 3139.145 | 2600 | 7148.075 | 4295.784 | 1.19E+00 | −2.72E−01 | 20 | Other | |
| | | marker 1 prec | | | | | | | | | |
| Q04941 | O16ab8 | proteolipid protein | 17951.73 | 11800 | 8567.736 | 12775.93 | −1.07E+00 | −6.04E−01 | 20 | Other | |
| | | 2 (colonic epit | | | | | | | | | |
| Q9UP11 | M14cd2 | small nuclear | 4625.515 | 8280 | 8998.787 | 7300.387 | 9.60E−01 | 8.39E−01 | 20 | Other | |
| | | ribonucleoprotein | | | | | | | | | |
| P13987 | B18ef7 | CD59 antigen p 18-20 | 17383.17 | 19200 | 10453.43 | 15673.38 | −7.34E−01 | 1.42E−01 | 20 | Other | |
| | | (antigen id | | | | | | | | | |
| P15951 | N04cd2 | protease; serine; 3 | 74972.46 | 7610 | 11417.5 | 31332.74 | −2.72E+00 | −3.30E+00 | 20 | Other | |
| | | (trypsin 3) | | | | | | | | | |
| P04687 | P12cd1 | tubulin; alpha; | 15943.82 | 7260 | 15413.44 | 12871.67 | −4.88E−02 | −1.14E+00 | 20 | Other | |
| | | ubiquitous | | | | | | | | | |
| P04687 | H12cd1 | tubulin; alpha; | 15832.93 | 7590 | 24085.35 | 15834.49 | 6.05E−01 | −1.06E+00 | 20 | Other | |
| | | ubiquitous | | | | | | | | | |
| Q9UI09 | L02gh4 | 13kDa differentiation- | 1754.698 | 1990 | 2889.148 | 2210.196 | 7.19E−01 | 1.79E−01 | 3 | Unchanged High | |
| | | associated | | | | | | | | | |
| Q9Y6H1 | E23ef2 | 16.7KD protein | 5687.333 | 4050 | 5837.94 | 5191.628 | 3.77E−02 | −4.90E−01 | 3 | Unchanged High | |
| Q9P0U1 | C07gh3 | 6.2kD protein | 2323.402 | 1990 | 3847.586 | 2721.439 | 7.28E−01 | −2.21E−01 | 3 | Unchanged High | |
| P80095 | N10ab7 | 6-pyruvoyl- | 3223.922 | 2820 | 1569.69 | 2471.592 | −1.04E+00 | −2.99E−01 | 3 | Unchanged High | |
| | | tetrahydropterin synt | | | | | | | | | |
| Q92747 | F04ab2 | actin related protein | 4894.401 | 3990 | 3030.018 | 3972.89 | −6.92E−01 | −2.93E−01 | 3 | Unchanged High | |
| | | 2/3 complex | | | | | | | | | |
| O15144 | F08ab2 | actin related protein | 13976.76 | 21400 | 17206.31 | 17538.09 | 3.00E−01 | 6.17E−01 | 3 | Unchanged High | |
| | | 2/3 complex | | | | | | | | | |
| O15145 | F10ab2 | actin related protein | 3065.881 | 3190 | 1793.813 | 2683.638 | −7.73E−01 | 5.78E−02 | 3 | Unchanged High | |
| | | 2/3 complex | | | | | | | | | |
| P02570 | A01cd7 | actin; beta | 66304.81 | 45400 | 38777.92 | 50171.41 | −7.74E−01 | −5.45E−01 | 3 | Unchanged High | |
| P02570 | H12cd7 | actin; beta | 64949.74 | 42400 | 44598.66 | 50643.44 | −5.42E−01 | −6.16E−01 | 3 | Unchanged High | |
| P02570 | P12cd7 | actin; beta | 21347.36 | 12200 | 15408.61 | 16331.85 | −4.70E−01 | −8.03E−01 | 3 | Unchanged High | |
| P02571 | A06ab6 | actin: gamma 1 | 69165.16 | 59100 | 65677.15 | 64643.45 | −7.47E−02 | −2.27E−01 | 3 | Unchanged High | |
| P12814 | E23ab2 | actinin; alpha 1 | 2400.607 | 2160 | 2638.011 | 2400.844 | 1.36E−01 | −1.50E−01 | 3 | Unchanged High | |
| O43707 | G05ab2 | actinin: alpha 4 | 6999.138 | 3810 | 3499.234 | 4769.196 | −1.00E+00 | −8.78E−01 | 3 | Unchanged High | |
| P53999 | C05cd7 | activated RNA | 4478.986 | 4320 | 6009.318 | 4936.98 | 4.24E−01 | −5.13E−02 | 3 | Unchanged High | |
| | | polymerase II tran | | | | | | | | | |
| P18848 | I03et5 | activating transcription | 7160.888 | 5420 | 7668.286 | 6749.873 | 9.88E−02 | −4.02E−01 | 3 | Unchanged High | |
| | | factor 4 (t | | | | | | | | | |
| P53680 | A07ef1 | adaptor-related protein | 2255.235 | 2530 | 4102.633 | 2963.423 | 8.63E−01 | 1.67E−01 | 3 | Unchanged High | |
| | | complex | | | | | | | | | |
| P29274 | A18et5 | adenosine A2a | 1990.124 | 2960 | 2004.596 | 2318.367 | 1.05E−02 | 5.73E−01 | 3 | Unchanged High | |
| | | receptor | | | | | | | | | |
| P54819 | A04ab2 | adenylate kinase 2 | 2997.607 | 2520 | 2851.739 | 2788.3 | −7.20E−02 | −2.53E−01 | 3 | Unchanged High | |
| Q01518 | O18cd6 | adenylyl cyclase- | 6946.788 | 11000 | 7171.205 | 8362.375 | 4.59E−02 | 6.59E−01 | 3 | Unchanged High | |
| | | associated prot | | | | | | | | | |
| P32889 | C03ef6 | ADP-ribosylation | 6040.796 | 7220 | 7907.02 | 7056.851 | 3.88E−01 | 2.58E−01 | 3 | Unchanged High | |
| | | factor 1 | | | | | | | | | |
| P18085 | P21ab2 | ADP-ribosylation | 4777.904 | 8880 | 8342.165 | 7331.81 | 8.04E−01 | 8.93E−01 | 3 | Unchanged High | |
| | | factor 4 | | | | | | | | | |
| P26437 | B02ab2 | ADP-ribosylation | 3739.477 | 4430 | 3984.845 | 4052.262 | 9.17E−02 | 2.45E−01 | 3 | Unchanged High | |
| | | factor 5 | | | | | | | | | |
| Q9BZX4 | L08gh6 | AKAP-binding sperm | 2345.455 | 2780 | 2500.118 | 2541.066 | 9.21E−02 | 2.44E−01 | 3 | Unchanged High | |
| | | protein rop | | | | | | | | | |
| P11766 | K01ab2 | alcohol dehydro- | 2220.156 | 1990 | 2854.318 | 2354.247 | 3.62E−01 | −1.59E−01 | 3 | Unchanged High | |
| | | genase 5 (class | | | | | | | | | |
| P05067 | N03ef6 | amyloid beta (A4) | 2846.85 | 4180 | 3918.595 | 3649.558 | 4.61E−01 | 5.55E−01 | 3 | Unchanged High | |
| | | precursor pro | | | | | | | | | |
| Q06481 | K22ab2 | amyloid beta (A4) | 2117.652 | 3230 | 2290.191 | 2546.598 | 1.13E−01 | 6.10E−01 | 3 | Unchanged High | |
| | | precursor-like | | | | | | | | | |
| P07355 | I12ab2 | annexin A2 | 74767.39 | 112000 | 97217.17 | 94734.13 | 3.79E−01 | 5.86E−01 | 3 | Unchanged High | |
| P08758 | I18ab2 | annexin A5 | 4487.905 | 4270 | 7134.202 | 5295.976 | 6.69E−01 | −7.32E−02 | 3 | Unchanged High | |
| P55064 | O08ab2 | aquaporin 5 | 10851.57 | 14200 | 11650.24 | 12248.87 | 1.02E−01 | 3.93E−01 | 3 | Unchanged High | |
| Q13520 | O10ab2 | aquaporin 6: | 2404.748 | 2350 | 2457.276 | 2404.397 | 3.12E−02 | −3.25E−02 | 3 | Unchanged High | |
| | | kidney specific | | | | | | | | | |
| P32391 | D19ab2 | ARP3 actin-related | 4291.823 | 4280 | 3941.05 | 4171.013 | −1.23E−01 | −3.92E−03 | 3 | Unchanged High | |
| | | protein 3 ho | | | | | | | | | |
| O43776 | E18ab7 | asparaginyl-tRNA | 2570.968 | 3010 | 2854.989 | 2811.634 | 1.51E−01 | 2.27E−01 | 3 | Unchanged High | |
| | | synthetase | | | | | | | | | |
| P24539 | N22ab2 | ATP synthase; H+ | 2908.697 | 3800 | 3228.203 | 3312.717 | 1.50E−01 | 3.86E−01 | 3 | Unchanged High | |
| | | transporting; | | | | | | | | | |
| P48201 | P04ab2 | ATP synthase; H+ | 5694.182 | 4370 | 4857.955 | 4974.135 | −2.29E−01 | −3.82E−01 | 3 | Unchanged High | |
| | | transporting; | | | | | | | | | |
| P05496 | N24ab2 | ATP synthase; H+ | 2176.378 | 1760 | 2400.575 | 2112.628 | 1.41E−01 | −3.06E−0l | 3 | Unchanged High | |
| | | transporting; | | | | | | | | | |
| Q06055 | P02ab2 | ATP synthase; H+ | 5784.579 | 3530 | 7732.552 | 5682.609 | 4.19E−01 | −7.12E−01 | 3 | Unchanged High | |
| | | transporting; | | | | | | | | | |
| O75947 | P14ab2 | ATP synthase; H+ | 2668.028 | 2520 | 1881.974 | 2355.858 | −5.04E−01 | −8.37E−02 | 3 | Unchanged High | |
| | | transporting; | | | | | | | | | |
| P56385 | P06ab2 | ATP synthase; H+ | 2682.798 | 2090 | 2616.726 | 2462.524 | −3.60E−02 | −3.62E−01 | 3 | Unchanged High | |
| | | transporting; | | | | | | | | | |
| P56134 | P10ab2 | ATP synthase; H+ | 11587.91 | 17200 | 14181.18 | 14327.12 | 2.91E−01 | 5.71E−01 | 3 | Unchanged High | |
| | | transporting; | | | | | | | | | |
| P18859 | P08ab2 | ATP synthase; H+ | 3879.939 | 4840 | 5176.585 | 4633.559 | 4.16E−01 | 3.20E−01 | 3 | Unchanged High | |
| | | transporting; | | | | | | | | | |
| O75964 | P16ab2 | ATP synthase; H+ | 2021.591 | 2630 | 2009.026 | 2219.11 | −8.99E−03 | 3.78E−01 | 3 | Unchanged High | |
| | | transporting; | | | | | | | | | |
| P25705 | N14ab2 | ATP synthase; H+ | 3657.512 | 2410 | 2967.207 | 3009.915 | −3.02E−01 | −6.05E−01 | 3 | Unchanged High | |
| | | transporting; | | | | | | | | | |
| P06576 | N16ab2 | ATP synthase; H+ | 4072.978 | 3280 | 2557.962 | 3305.047 | −6.71E−01 | −3.11E−01 | 3 | Unchanged High | |
| | | transporting; | | | | | | | | | |
| P36542 | N18ab2 | ATP synthase; H+ | 2479.5 | 2030 | 2436.284 | 2313.989 | −2.54E−02 | −2.91E−01 | 3 | Unchanged High | |
| | | transporting; | | | | | | | | | |
| P48047 | P18ab2 | ATP synthase; H+ | 4222.314 | 4100 | 3541.047 | 3955.838 | −2.54E−01 | −4.09E−02 | 3 | Unchanged High | |
| | | transporting; | | | | | | | | | |
| Q01814 | P01ef5 | ATPase; Ca++ | 3157.447 | 3180 | 3242.707 | 3193.535 | 3.84E−02 | 1.05E−02 | 3 | Unchanged High | |
| | | transporting; plas | | | | | | | | | |
| Q99437 | A11ab3 | ATPase; H+ | 2495.618 | 3870 | 2087.291 | 2817.803 | −2.58E−01 | 6.33E−01 | 3 | Unchanged High | |
| | | transporting; lysoso | | | | | | | | | |
| P36543 | A09ab3 | ATPase; H+ | 1854.198 | 2920 | 1659.749 | 2145.908 | −1.60E−01 | 6.57E−01 | 3 | Unchanged High | |
| | | transporting; lysoso | | | | | | | | | |
| O75348 | A15ab3 | ATPase; H+ | 2734.484 | 5660 | 2721.678 | 3704.42 | −6.67E−03 | 1.05E+00 | 3 | Unchanged High | |
| | | transporting; lysoso | | | | | | | | | |
| Q16864 | A21ab3 | ATPase; H+ | 6452.29 | 15900 | 7960.234 | 10101.59 | 3.03E−01 | 1.30E+00 | 3 | Unchanged High | |
| | | transporting; lysoso | | | | | | | | | |
| P27449 | A05ab3 | ATPase; H+ | 17134.14 | 24300 | 13120.57 | 18185.04 | −3.85E−01 | 5.04E−01 | 3 | Unchanged High | |
| | | transporting; lysoso | | | | | | | | | |
| O00244 | L14ab2 | ATX1 antioxidant | 2833.957 | 3500 | 3977.547 | 3435.675 | 4.89E−01 | 3.03E−01 | 3 | Unchanged High | |
| | | protein 1 homo | | | | | | | | | |
| O14503 | M19ab3 | basic helix-loop- | 4978.916 | 4280 | 3254.631 | 4169.761 | −6.13E−01 | −2.20E−01 | 3 | Unchanged High | |
| | | helix domain co | | | | | | | | | |
| O60238 | L11ab5 | BCL2/adenovirus | 1473.428 | 2420 | 3224.141 | 2371.407 | 1.13E+00 | 7.14E−01 | 3 | Unchanged High | |
| | | E1B 19kD inte | | | | | | | | | |
| P30536 | L21ab3 | benzodiazapine | 1997.929 | 2890 | 2326.211 | 2405.625 | 2.19E−01 | 5.34E−01 | 3 | Unchanged High | |
| | | receptor (periph | | | | | | | | | |
| P01884 | C23ab3 | beta-2-microglobulin | 64872 | 105000 | 36055.95 | 68478.91 | −8.47E−01 | 6.88E−01 | 3 | Unchanged High | |
| P22004 | I13ab5 | bone morphogenetic | 5036.985 | 4710 | 3780.005 | 4509.317 | −4.14E−01 | −9.65E−02 | 3 | Unchanged High | |
| | | protein 6 | | | | | | | | | |
| O95415 | B02ef3 | brain protein 13 | 7498.734 | 11200 | 5348.84 | 8032.295 | −4.87E−01 | 5.85E−01 | 3 | Unchanged High | |
| O75531 | K11ab3 | Breakpoint cluster | 2541.789 | 2740 | 2924.826 | 2736.992 | 2.03E−01 | 1.11E−01 | 3 | Unchanged High | |
| | | region protein | | | | | | | | | |
| P02593 | A10ab6 | calmodulin 2 | 1573.77 | 4130 | 4742.485 | 3482.215 | 1.59E+00 | 1.39E+00 | 3 | Unchanged High | |
| | | (phosphorylase kin | | | | | | | | | |
| P07384 | O24ef1 | calpain 1; (mu/l) | 3429.132 | 3420 | 2390.011 | 3078.324 | −5.21E−01 | −5.61E−03 | 3 | Unchanged High | |
| | | large subunit | | | | | | | | | |
| P27797 | P05ab5 | calreticulin | 10259.42 | 9700 | 15437.35 | 11797.33 | 5.89E−01 | −8.16E−02 | 3 | Unchanged High | |
| | N06gh8 | CaM-KII | 20298.71 | 28200 | 21760.79 | 23425.99 | 1.00E−01 | 4.75E−01 | 3 | Unchanged High | |
| | | inhibitory protein | | | | | | | | | |
| P47756 | N14ab4 | capping protein | 4348.079 | 4450 | 5015.377 | 4604.9 | 2.06E−01 | 3.38E−02 | 3 | Unchanged High | |
| | | (actin filament) n | | | | | | | | | |
| P15086 | H13ab5 | carboxypeptidase | 9766.062 | 27.1 | 26.97129 | 3273.378 | −8.50E+00 | −8.49E+00 | 3 | Unchanged High | |
| | | B1 (tissue) | | | | | | | | | |
| P52952 | D22ab5 | cardiac-specific | 2859.197 | 1740 | 2282.546 | 2292.94 | −3.25E−01 | −7.19E−01 | 3 | Unchanged High | |
| | | homeo box | | | | | | | | | |
| P13862 | M21ef1 | casein kinase 2; | 3674.921 | 3840 | 3873.228 | 3795.282 | 7.58E−02 | 6.25E−02 | 3 | Unchanged High | |
| | | beta polypeptide | | | | | | | | | |
| O14675 | I02ef6 | CASP8 and FADD- | 2622.586 | 2500 | 1151.661 | 2092.229 | −1.19E+00 | −6.77E−02 | 3 | Unchanged High | |
| | | like apoptosis | | | | | | | | | |
| P35221 | C17ef7 | catenin (cadherin- | 3813.516 | 4230 | 3300.738 | 3782.772 | −2.08E−01 | 1.51E−01 | 3 | Unchanged High | |
| | | associated pro | | | | | | | | | |
| P07858 | E01a5 | cathepsin B | 2480.797 | 3860 | 4485.422 | 3607.853 | 8.54E−01 | 6.37E−01 | 3 | Unchanged High | |
| P48509 | K16ab4 | CD151 antigen | 7267.56 | 13700 | 8768.731 | 9901.71 | 2.71E−01 | 9.11E−01 | 3 | Unchanged High | |
| P25063 | G22cd8 | CD24 antigen | 21319.27 | 18200 | 8278.973 | 15932.79 | −1.36E+00 | −2.28E−01 | 3 | Unchanged High | |
| | | (small cell lung ca | | | | | | | | | |
| P16070 | O21ef1 | CD44 antigen | 16178.96 | 9640 | 5778.642 | 10533.43 | −1.49E+00 | −7.47E−01 | 3 | Unchanged High | |
| | | (homing function a | | | | | | | | | |
| P08962 | O18ab4 | CD63 antigen | 9354.914 | 11600 | 10016.29 | 10307.1 | 9.66E−02 | 3.04E−01 | 3 | Unchanged High | |
| | | (melanoma 1 antig | | | | | | | | | |
| O14519 | K04ef5 | CDK2-associated | 14231.191 | 4180 | 4763.013 | 4389.995 | 1.71E−01 | −1.90E−02 | 3 | Unchanged High | |
| | | protein | | | | | | | | | |
| P25763 | A12ab6 | cell division cycle | 4588.819 | 2840 | 2852.186 | 3426.736 | −6.86E−01 | −6.93E−01 | 3 | Unchanged High | |
| | | 42 (GTP bindi | | | | | | | | | |
| P49368 | N17cd2 | chaperonin | 2356.627 | 2880 | 2222.361 | 2484.939 | −8.46E−02 | 2.87E−01 | 3 | Unchanged High | |
| | | containing TCP1; su | | | | | | | | | |
| P48643 | H10ef4 | chaperonin containing | 2236.933 | 2330 | 1561.625 | 2042.12 | −5.20E−01 | 5.49E−02 | 3 | Unchanged High | |
| | | TCP1; su | | | | | | | | | |
| O00299 | M19ab6 | chloride intracellular | 6811.55 | 7240 | 5727.121 | 6591.299 | −2.50E−01 | 8.71E−02 | 3 | Unchanged High | |
| | | channel 1 | | | | | | | | | |
| Q9Y2Q7 | N02cd8 | chromosome 11 open | 4087.361 | 3800 | 5946.259 | 4610.942 | 5.41E−01 | −1.05E−01 | 3 | Unchanged High | |
| | | reading fra | | | | | | | | | |
| P09496 | F15ab5 | clathrin; light polypeptide | 5758.632 | 9260 | 5849.641 | 6957.341 | 2.26E−02 | 6.86E−01 | 3 | Unchanged High | |
| | | (Lca) | | | | | | | | | |
| O95832 | L04gh1 | claudin 1 | 3046.376 | 2450 | 918.0838 | 2138.383 | −1.73E+00 | −3.14E−01 | 3 | Unchanged High | |
| O95471 | A13ef1 | claudin 7 | 2888.819 | 3350 | 1520.977 | 2587.177 | −9.2SE−01 | 2.14E−01 | 3 | Unchanged High | |
| P23528 | M20ab5 | cofilin 1 (non-muscle) | 16021.95 | 18700 | 14063.43 | 16255.64 | −1.88E−01 | 2.22E−01 | 3 | Unchanged High | |
| P16989 | L08ef6 | cold shock domain | 2942.723 | 2530 | 2069.184 | 2515.212 | −5.08E−01 | −2.16E−01 | 3 | Unchanged High | |
| | | protein A | | | | | | | | | |
| P38936 | O21ef5 | cyclin-dependent | 3643.18 | 4250 | 3437.076 | 3777.57 | −8.40E−02 | 2.23E−01 | 3 | Unchanged High | |
| | | kinase inhibitor | | | | | | | | | |
| Q9UII4 | B14ef1 | cyclin-E binding | 26180.8 | 29800 | 39655.02 | 31877.4 | 5.992−01 | 1.87E−01 | 3 | Unchanged High | |
| | | protein 1 | | | | | | | | | |
| P04080 | G16ab3 | cystatin B | 4312.168 | 5420 | 2370.13 | 4034.272 | −8.632−01 | 3.30E−01 | 3 | Unchanged High | |
| | | (stefin B) | | | | | | | | | |
| P21291 | J01ab5 | cysteine and glycine- | 2478.366 | 1710 | 2135.86 | 2109.096 | −2.15E−01 | −5.33E−01 | 3 | Unchanged High | |
| | | rich protein | | | | | | | | | |
| O00622 | J15ef7 | cysteine-rich: | 2070.203 | 1620 | 2978.542 | 2224.339 | 5.25E−01 | −3.50E−01 | 3 | Unchanged High | |
| | | angiogenic induce | | | | | | | | | |
| P13073 | L19ab5 | cytochrome c oxidase | 6219.628 | 6550 | 6571.848 | 7115.048 | 7.95E−02 | 4.60E−01 | 3 | Unchanged High | |
| | | subunit IV | | | | | | | | | |
| P12074 | K13gh1 | cytochrome c oxidase | 3253.24 | 5800 | 4244.557 | 4434.13 | 3.842−01 | 8.35E−01 | 3 | Unchanged High | |
| | | subunit VI | | | | | | | | | |
| Q02221 | E17ab6 | cytochrome c oxidase | 2968.24 | 2820 | 4604.241 | 3464.904 | 6.33E−01 | −7.28E−02 | 3 | Unchanged High | |
| | | subunit VI | | | | | | | | | |
| P14854 | A20ab6 | cytochrome c oxidase | 1991.91 | 2660 | 2380.795 | 2344.331 | 2.57E−01 | 4.17E−01 | 3 | Unchanged High | |
| | | subunit VI | | | | | | | | | |
| P09669 | E12ab6 | cytochrome c oxidase | 2580.598 | 2090 | 1927.002 | 2197.58 | −4.21E−01 | −3.08E−01 | 3 | Unchanged High | |
| | | subunit VI | | | | | | | | | |
| P14406 | G02ab5 | cytochrome c oxidase | 4846.713 | 4500 | 4245.478 | 4530.346 | −1.91E−01 | −1.07E−01 | 3 | Unchanged High | |
| | | subunit VI | | | | | | | | | |
| O14548 | G23cd5 | cytochrome c oxidase | 2182.954 | 2450 | 3040.580 | 2556.481 | 4.78E−01 | 1.64E−01 | 3 | Unchanged High | |
| | | subunit VI | | | | | | | | | |
| P24311 | K17ab5 | cytochrome c oxidase | 3224.558 | 5150 | 3801.752 | 4057.995 | 2.38E−01 | 6.75E−01 | 3 | Unchanged High | |
| | | subunit VI | | | | | | | | | |
| P15954 | P04ab5 | cytochrome c oxidase | 12335.74 | 12200 | 9883.688 | 11482.48 | −3.20E−01 | −1.27E−02 | 3 | Unchanged High | |
| | | subunit VI | | | | | | | | | |
| P10176 | B10ab5 | cytochrome c oxidase | 3019.162 | 1900 | 3329.261 | 2750.974 | 1.41E−01 | −6.65E−01 | 3 | Unchanged High | |
| | | subunit VI | | | | | | | | | |
| O43293 | K19ab4 | death-associated | 2535.403 | 1650 | 2088.808 | 2157.783 | −2.60E−01 | −4.55E−01 | 3 | Unchanged High | |
| | | protein kinase | | | | | | | | | |
| P46966 | G14eF6 | defender against | 7921.682 | 11200 | 9546.896 | 9563.241 | 2.69E−01 | 5.02E−01 | 3 | Unchanged High | |
| | | cell death 1 | | | | | | | | | |
| Q09753 | G01ab6 | defensin; beta 1 | 4071.982 | 7630 | 2153.638 | 4619.611 | −9.19E−01 | 9.07E−01 | 3 | Unchanged High | |
| P18282 | F23ab2 | destrin (actin | 7729.126 | 7570 | 6083.408 | 7126.01 | −3.45E−01 | −3.09E−02 | 3 | Unchanged High | |
| | | depolymerizing fac | | | | | | | | | |
| P07108 | H04gh1 | diazepam binding | 2541.011 | 1730 | 2995.768 | 2422.578 | 2.38E−01 | −5.54E−01 | 3 | Unchanged High | |
| | | inhibitor (GAB | | | | | | | | | |
| P31689 | D18ab7 | DnaJ (Hsp40) homolog, | 2491.051 | 2930 | 1789.662 | 2402.398 | −4.77E−01 | 2.32E−01 | 3 | Unchanged High | |
| | | subfamil | | | | | | | | | |
| Q9Y5T4 | I10cd8 | DNAJ domain-containing | 2324.933 | 2520 | 2397.896 | 2414.457 | 4.46E−02 | 1.17E−01 | 3 | Unchanged High | |
| Q9Y463 | G15cd5 | dual-specificity | 8211.965 | 6870 | 5987.39 | 7023.599 | −4.56E−01 | −2.57E−01 | 3 | Unchanged High | |
| | | tyrosine-(Y)-phos | | | | | | | | | |
| Q9NP97 | C02ef8 | dynein light chain 2A | 4080.949 | 4160 | 5365.926 | 4534.283 | 3.95E−01 | 2.63E−02 | 3 | Unchanged High | |
| Q15701 | L08cd3 | dynein: cytoplasmic: | 4492.679 | 5800 | 4332.161 | 4875.665 | −5.25E−02 | 3.69E−01 | 3 | Unchanged High | |
| | | light polype | | | | | | | | | |
| P78545 | O16ab4 | E74-like factor 3 | 3781.196 | 2780 | 1455.944 | 2673.744 | −1.38E+00 | −4.42E−01 | 3 | Unchanged High | |
| | | (ets domain trar | | | | | | | | | |
| O60869 | A03cd4 | endothelial | 5515.692 | 5680 | 4090.527 | 5094.856 | −4.31E−01 | 4.19E−02 | 3 | Unchanged High | |
| | | differentiation-related | | | | | | | | | |
| Q14259 | L06ab4 | enhancer of | 3593.608 | 3950 | 5175.131 | 4241.038 | 5.26E−01 | 1.38E−01 | 3 | Unchanged High | |
| | | rudimentary homolo | | | | | | | | | |
| P06733 | C16ab4 | enolase 1; (alpha) | 12539.97 | 7460 | 9955.024 | 9983.774 | −3.33E−01 | −7.50E−01 | 3 | Unchanged High | |
| P22712 | D11gh6 | enolase 1; (alpha) | 6900.857 | 4290 | 5016.695 | 5403.444 | −4.60E−01 | −6.85E−01 | 3 | Unchanged High | |
| P04720 | O07ab6 | eukaryotic translation | 36222.34 | 23200 | 23485.94 | 27648.74 | −6.25E−01 | −6.40E−01 | 3 | Unchanged High | |
| | | elongation | | | | | | | | | |
| P26641 | A10ab4 | eukaryotic translation | 6863.565 | 6540 | 11607.61 | 8337.901 | 7.58E−01 | −6.91E−02 | 3 | Unchanged High | |
| | | elongation | | | | | | | | | |
| P13639 | I15ab5 | eukaryotic translation | 4023.855 | 2310 | 3360.073 | 3231.325 | −2.60E−01 | −8.01E−01 | 3 | Unchanged High | |
| | | elongation | | | | | | | | | |
| O15372 | L24cd3 | eukaryotic translation | 2070.027 | 1930 | 2391.869 | 2398.394 | −2.63E−01 | −5.70E−01 | 3 | Unchanged High | |
| | | initiation fa | | | | | | | | | |
| O75821 | L22cd3 | eukaryotic translation | 3009.075 | 2670 | 3333.728 | 3005.577 | 1.48E−01 | −1.70E−01 | 3 | Unchanged High | |
| | | initiation fa | | | | | | | | | |
| Q64252 | L11ab6 | eukaryotic translation | 2755.438 | 1540 | 2127.674 | 2141.68 | −3.73E−01 | −6.38E−01 | 3 | Unchanged High | |
| | | initiation fa | | | | | | | | | |
| O15371 | L18cd3 | eukaryotic translation | 4273.919 | 4030 | 3558.838 | 3954.374 | −2.64E−01 | −8.46E−02 | 3 | Unchanged High | |
| | | initiation fa | | | | | | | | | |
| P04765 | A22ab4 | eukaryotic translation | 7716.222 | 7170 | 6363.169 | 7083.99 | −2.78E−01 | −1.05E−01 | 3 | Unchanged High | |
| | | initiation fa | | | | | | | | | |
| Q14240 | H13ab4 | eukaryotic translation | 2091.816 | 2190 | 2519.322 | 2267.015 | 2.68E−01 | 6.61E−02 | 3 | Unchanged High | |
| | | initiation fa | | | | | | | | | |
| P10159 | A18ab5 | eukaryotic translation | 9023.244 | 5240 | 5651.752 | 6638.65 | −6.75E−01 | −7.84E−01 | 3 | Unchanged High | |
| | | initiation fa | | | | | | | | | |
| Q9NQT4 | H05gh4 | exosome component | 2098.514 | 2590 | 2304.48 | 2331.056 | 1.35E−01 | 3.04E−01 | 3 | Unchanged High | |
| | | Rrp46 | | | | | | | | | |
| P02794 | C12ab6 | ferritin; heavy | 35912.81 | 48700 | 42075.22 | 42243.59 | 2.28E−01 | 4.41E−01 | 3 | Unchanged High | |
| | | polypeptide 1 | | | | | | | | | |
| P02792 | M10ab3 | ferritin: light | 44225.86 | 29600 | 17849.2 | 30568.13 | −1.31E+00 | −5.78E−01 | 3 | Unchanged High | |
| | | polypeptide | | | | | | | | | |
| Q05472 | B02ab4 | Finkel-Biskis-Reilly | 10322.94 | 10400 | 9704.642 | 10145.46 | −8.91E−02 | 1.19E−02 | 3 | Unchanged High | |
| | | murine sarco | | | | | | | | | |
| P20071 | F08ab3 | FK506 binding protein | 2741.389 | 1870 | 1414.969 | 2009.291 | −9.54E−01 | −5.51E−01 | 3 | Unchanged High | |
| | | 1A (12kD) | | | | | | | | | |
| P50395 | J02ab4 | GDP dissociation | 2287.713 | 1580 | 2291.27 | 2051.41 | 2.24E−03 | −5.38E−01 | 3 | Unchanged High | |
| | | inhibitor 2 | | | | | | | | | |
| P48507 | E12ab5 | glutamate-cysteine | 154.1773 | 76.2 | 5937.375 | 2055.914 | 5.27E+00 | −1.02E+00 | 3 | Unchanged High | |
| | | ligase; modifi | | | | | | | | | |
| P36969 | D14ab5 | glutathione peroxidase | 3397.2 | 4860 | 3108.036 | 3787.13 | −1.28E−01 | 5.15E−01 | 3 | Unchanged High | |
| | | 4 (phosp | | | | | | | | | |
| P09211 | L10ab3 | glutathione S- | 6392.334 | 8670 | 4067.789 | 6378.198 | −6.52E−01 | 4.40E−01 | 3 | Unchanged High | |
| | | transferase pi | | | | | | | | | |
| P04406 | A01cd8 | glyceraldehyde-3- | 28809.37 | 38300 | 44655.67 | 37260.67 | 6.32E−01 | 4.11E−01 | 3 | Unchanged High | |
| | | phosphate deh | | | | | | | | | |
| P04406 | H12cd8 | glyceraldehyde-3- | 37562.25 | 60200 | 57135.41 | 51621.55 | 6.05E−01 | 6.80E−01 | 3 | Unchanged High | |
| | | phosphate deh | | | | | | | | | |
| P04406 | P12cd8 | glyceraldehyde-3- | 12053.5 | 14900 | 19061.62 | 15342.7 | 6.61E−01 | 3.07E−01 | 3 | Unchanged High | |
| | | phosphate deh | | | | | | | | | |
| P43304 | H01ab3 | glycerol-3- | 3450.634 | 2340 | 3276.264 | 3023.392 | −7.48E−02 | −5.58E−01 | 3 | Unchanged High | |
| | | phosphate dehydroge | | | | | | | | | |
| P41250 | F02ef7 | glycyl-tRNA | 4333.439 | 3630 | 6248.604 | 4736.632 | 5.28E−01 | −2.56E−01 | 3 | Unchanged High | |
| | | synthetase | | | | | | | | | |
| P04895 | K17ef5 | GNAS complex locus | 7755.858 | 9290 | 9107.558 | 8718.93 | 2.32E−01 | 2.61E−01 | 3 | Unchanged High | |
| P24522 | B03ef6 | growth arrest and | 2792.429 | 6130 | 3396.736 | 4105.138 | 2.83E−01 | 1.13E+00 | 3 | Unchanged High | |
| | | DNA-damage-i | | | | | | | | | |
| Q92847 | M24ab5 | growth hormone | 2827.902 | 2100 | 2597.123 | 2508.414 | −1.23E−01 | −4.29E−01 | 3 | Unchanged High | |
| | | secretagogue re | | | | | | | | | |
| P04901 | B01ef1 | guanine nucleotide | 5734.337 | 7610 | 5853.112 | 6398.85 | 2.96E−02 | 4.08E−01 | 3 | Unchanged High | |
| | | binding prote | | | | | | | | | |
| P25388 | E20cd8 | guanine nucleotide | 22430.28 | 15300 | 19055.7 | 18939.85 | −2.35E−01 | −5.49E−01 | 3 | Unchanged High | |
| | | binding prote | | | | | | | | | |
| P06351 | G08ab6 | H3 histone; | 2448.264 | 2770 | 2042.864 | 2421.658 | −2.61E−01 | 1.80E−01 | 3 | Unchanged High | |
| | | family 3B (H3.3B) | | | | | | | | | |
| P04792 | A16ef5 | heat shock 27kD | 41952.14 | 34500 | 31970.86 | 36135.19 | −3.92E−01 | −2.83E−01 | 3 | Unchanged High | |
| | | protein 1 | | | | | | | | | |
| P11021 | H01ef1 | heat shock 70kD | 3226.183 | 1600 | 4101.742 | 2976.537 | 3.46E−01 | −1.01E+00 | 3 | Unchanged High | |
| | | protein 5 (gluc | | | | | | | | | |
| P38646 | O19ab7 | heat shock 70kD | 2851.743 | 2180 | 2220.509 | 2417.791 | −3.61E−01 | −3.87E−01 | 3 | Unchanged High | |
| | | protein 9B (mor | | | | | | | | | |
| Q9UK76 | J14ef1 | hematological and | 26342.33 | 35400 | 41099.58 | 34286.74 | 6.42E−01 | 4.27E−01 | 3 | Unchanged High | |
| | | neurological | | | | | | | | | |
| P09651 | F12ab7 | heterogeneous nuclear | 2522.253 | 1650 | 2524.082 | 2231.413 | 1.05E−03 | −6.14E−01 | 3 | Unchanged High | |
| | | ribonucle | | | | | | | | | |
| Q9Y4J5 | I23ef1 | heterogeneous nuclear | 3825.912 | 2370 | 3770.457 | 3320.487 | −2.11E−02 | −6.94E−01 | 3 | Unchanged High | |
| | | ribonucle | | | | | | | | | |
| P09429 | F10ab7 | high-mobility group | 2534.273 | 1660 | 3502.047 | 2567.093 | 4.67E−01 | −6.06E−01 | 3 | Unchanged High | |
| | | (nonhistone | | | | | | | | | |
| P49773 | M03ef6 | histidine triad | 6025.978 | 7790 | 7379.267 | 7064.436 | 2.92E−01 | 3.70E−01 | 3 | Unchanged High | |
| | | nucleotide binding | | | | | | | | | |
| Q92769 | N05ab4 | histone deacetylase 2 | 2454.324 | 2220 | 1789.508 | 2155.575 | −4.56E−01 | −1.43E−01 | 3 | Unchanged High | |
| P17693 | F08ab7 | HLA-G histo- | 2048.009 | 2780 | 1844.177 | 2225.106 | −1.51E−01 | 4.42E−01 | 3 | Unchanged High | |
| | | compatibility antigen | | | | | | | | | |
| P17483 | C15gh2 | homeo box B4 | 2892.488 | 3490 | 2902.69 | 3095.004 | 5.08E−03 | 2.71E−01 | 3 | Unchanged High | |
| Q9UL99 | C01cd8 | hyalurono- | 3623.461 | 2050 | 2189.656 | 2621.286 | −7.27E−01 | −8.21E−01 | 3 | Unchanged High | |
| | | glucosaminidase 4 | | | | | | | | | |
| Q9GZT3 | L18gh7 | hypothetical protein | 3343.922 | 3750 | 4298.676 | 3797.943 | 3.62E−01 | 1.66E−01 | 3 | Unchanged High | |
| | | DC50 | | | | | | | | | |
| Q9NWY5 | O12gh2 | hypothetical protein | 3104.471 | 5460 | 6184.227 | 4915.419 | 9.94E−01 | 8.14E−01 | 3 | Unchanged High | |
| | | FLJ20533 | | | | | | | | | |
| Q9U130 | P08ef2 | hypothetical protein | 3000.217 | 4060 | 4396.862 | 3819.313 | 5.51E−01 | 4.37E−01 | 3 | Unchanged High | |
| | | HSPC152 | | | | | | | | | |
| Q9BQB6 | P08gh5 | hypothetical protein | 3599.509 | 5410 | 8687.307 | 5897.337 | 1.27E+00 | 5.87E−01 | 3 | Unchanged High | |
| | | IMAGE3455: | | | | | | | | | |
| Q9BWJ5 | P18gh7 | hypothetical protein | 2074.073 | 2520 | 2719.671 | 2436.767 | 3.91E−01 | 2.79E−01 | 3 | Unchanged High | |
| | | MGC3133 | | | | | | | | | |
| Q9Y683 | N11ef2 | hypothetical protein | 2425.475 | 2730 | 2939.253 | 2699.545 | 2.77E−01 | 1.73E−01 | 3 | Unchanged High | |
| | | MGC8721 | | | | | | | | | |
| Q16665 | P01ef6 | hypoxia-inducible | 3544.753 | 2960 | 1526.935 | 2675.666 | −1.22E+00 | −2.62E−01 | 3 | Unchanged High | |
| | | factor 1; alpha | | | | | | | | | |
| O75353 | G04ef6 | immediate early | 4564.408 | 4690 | 3129.721 | 4129.098 | −5.44E−01 | 4.01E−02 | 3 | Unchanged High | |
| | | response 3 | | | | | | | | | |
| Q16270 | J15ab0 | insulin-like growth | 6185.622 | 21800 | 18047.34 | 15334.47 | 1.54E+00 | 1.82E+00 | 3 | Unchanged High | |
| | | factor binding | | | | | | | | | |
| Q9Y287 | A03gh2 | integral membrane | 6908.997 | 10500 | 10014.47 | 9139.933 | 5.36E−01 | 6.03E−01 | 3 | Unchanged High | |
| | | protein 2B | | | | | | | | | |
| P56537 | D16ab6 | integrin beta 4 | 2594.316 | 3270 | 1568.747 | 2476.062 | −7.26E−01 | 3.32E−01 | 3 | Unchanged High | |
| | | binding protein | | | | | | | | | |
| P26006 | D08ab6 | integrin; alpha 3 | 2669.777 | 2740 | 1339.712 | 2249.026 | −9.95E−01 | 3.62E−02 | 3 | Unchanged High | |
| | | (antigen CD49C | | | | | | | | | |
| P05556 | G01ef7 | integrin; beta 1 | 6743.428 | 5390 | 6511.579 | 6216.314 | −5.05E−02 | −3.22E−01 | 3 | Unchanged High | |
| | | (fibronectin recep | | | | | | | | | |
| Q01628 | C09gh7 | interferon induced | 14426.62 | 14800 | 8827.997 | 12669.24 | −7.09E−01 | 3.23E−02 | 3 | Unchanged High | |
| | | transmembrar | | | | | | | | | |
| Q12905 | C10ab7 | interleukin enhancer | 3045.484 | 2420 | 2596.915 | 2687.538 | −2.30E−01 | −3.32E−01 | 3 | Unchanged High | |
| | | binding fact | | | | | | | | | |
| Q13907 | A24ab7 | isopentenyl-diphosphate | 2958.139 | 1660 | 2475.365 | 2362.933 | −2.57E−01 | −8.38E−01 | 3 | Unchanged High | |
| | | delta is | | | | | | | | | |
| P33947 | K03cd7 | KDEL (Lys-Asp-Glu-Leu) | 1667.246 | 1250 | 3924.993 | 2281.692 | 1.24E+00 | −4.12E−01 | 3 | Unchanged High | |
| | | endop | | | | | | | | | |
| P00338 | D15ab7 | lactate dehydrogenase A | 44081.29 | 34500 | 42150.03 | 40260.24 | −6.46E−02 | −3.52E−01 | 3 | Unchanged High | |
| P08865 | G21ef7 | laminin receptor 1 | 8548.245 | 0070 | 9921.409 | 8180.607 | 2.15E−01 | −4.93E−01 | 3 | Unchanged High | |
| | | (67kD; riboso | | | | | | | | | |
| Q08380 | D17ab7 | lectin: galactoside- | 2294.491 | 2850 | 3014.02 | 2719.794 | 3.94E−01 | 3.13E−01 | 3 | Unchanged High | |
| | | binding; solub | | | | | | | | | |
| O14949 | F17ef3 | low molecular mass | 2942.579 | 3550 | 3637.463 | 3377.305 | 3.06E−01 | 2.71E−01 | 3 | Unchanged High | |
| | | ubiquinone-t | | | | | | | | | |
| Q15012 | F20ef4 | lysosomal-associated | 2815.002 | 4690 | 5453.634 | 4319.567 | 9.54E−01 | 7.36E−01 | 3 | Unchanged High | |
| | | protein tra | | | | | | | | | |
| P14174 | J05ef7 | macrophage migration | 5544.722 | 6700 | 6783.718 | 6337.062 | 2.87E−01 | 2.74E−01 | 3 | Unchanged High | |
| | | inhibitory | | | | | | | | | |
| P49006 | M05ef6 | macrophage myristoylated | 5857.382 | 10400 | 8986.315 | 8400.725 | 6.17E−01 | 8.22E−01 | 3 | Unchanged High | |
| | | alani | | | | | | | | | |
| P30463 | G10ab6 | major histo- | 2701.757 | 4890 | 2255.929 | 3281.592 | −2.60E−01 | 8.55E−01 | 3 | Unchanged High | |
| | | compatibility complex; | | | | | | | | | |
| P10321 | A01cd2 | major histo- | 8949.086 | 17000 | 10196.88 | 12044.89 | 1.88E−01 | 9.25E−01 | 3 | Unchanged High | |
| | | compatibility complex; | | | | | | | | | |
| P10321 | H12cd2 | major histo- | 7187.905 | 21600 | 11111.54 | 13298.19 | 6.28E−01 | 1.59E+00 | 3 | Unchanged High | |
| | | compatibility complex; | | | | | | | | | |
| P10321 | P12cd2 | major histo- | 13121.35 | 16800 | 11725.68 | 13868.02 | −1.62E−01 | 3.53E−01 | 3 | Unchanged High | |
| | | compatibility complex; | | | | | | | | | |
| P13747 | M24ab7 | major histo- | 2540.911 | 2790 | 2053.351 | 2460.503 | −3.07E−01 | 1.33E−01 | 3 | Unchanged High | |
| | | compatibility complex; | | | | | | | | | |
| Q95HC0 | P02gh6 | major histo- | 5067.294 | 4140 | 3295.115 | 4168.835 | −6.21E−01 | −2.90E−01 | 3 | Unchanged High | |
| | | compatibility complex; | | | | | | | | | |
| P13640 | D13gh6 | metallothionein 1G | 6180.376 | 4750 | 2940.683 | 4624.849 | −1.07E+00 | 3.79E−01 | 3 | Unchanged High | |
| P80297 | D17gh6 | metallothionein 1X | 4881.982 | 5310 | 5233.749 | 5142.996 | 1.00E−01 | 1.22E−01 | 3 | Unchanged High | |
| P02795 | L04ab7 | metallothionein 2A | 6620.235 | 7330 | 8067.274 | 7340.215 | 2.85E−01 | 1.48E−01 | 3 | Unchanged High | |
| O14880 | E04ab7 | microsomal glutathione | 5023.542 | 7940 | 4177.165 | 5712.043 | −2.66E−01 | 6.60E−01 | 3 | Unchanged High | |
| | | S-transfe | | | | | | | | | |
| Q9NYZ2 | M22ef2 | mitochondrial solute | 2488.07 | 2850 | 2240.824 | 2527.436 | −1.51E−01 | 1.98E−01 | 3 | Unchanged High | |
| | | carrier | | | | | | | | | |
| P26038 | K05ab7 | moesin | 4260.51 | 4580 | 3801.298 | 4212.427 | −1.65E−01 | 1.03E−01 | 3 | Unchanged High | |
| O15329 | O12ef1 | mucin 6: gastric | 5574.093 | 5950 | 6685.047 | 6068.269 | 2.62E−01 | 9.31E−02 | 3 | Unchanged High | |
| P16475 | J02gh1 | myosin; light | 48122.62 | 45200 | 48882.54 | 47407.02 | 2.26E−02 | −8.99E−02 | 3 | Unchanged High | |
| | | polypeptide 6; alkal | | | | | | | | | |
| P19105 | N03cd6 | myosin; light | 7920.873 | 8150 | 7228.401 | 7764.994 | −1.32E−01 | 4.04E−02 | 3 | Unchanged High | |
| | | polypeptide; regula | | | | | | | | | |
| Q9UK23 | D06ef1 | N-acetylglucosamine- | 1884.596 | 2680 | 2511.293 | 2357.087 | 4.14E−01 | 5.05E−01 | 3 | Unchanged High | |
| | | 1-phospho | | | | | | | | | |
| O15239 | E22ab7 | NADH dehydrogenase | 2859.738 | 5440 | 3719.503 | 4005.894 | 3.79E−01 | 9.27E−01 | 3 | Unchanged High | |
| | | (ubiguino | | | | | | | | | |
| O00483 | J02ab7 | NADH dehydrogenase | 5214.222 | 5540 | 9086.615 | 6613.046 | 8.01E−01 | 8.70E−02 | 3 | Unchanged High | |
| | | (ubiquino | | | | | | | | | |
| O75438 | J04ab8 | NADH dehydrogenase | 1628.024 | 2170 | 2842.647 | 2212.932 | 8.04E−01 | 4.13E−01 | 3 | Unchanged High | |
| | | (ubiguino | | | | | | | | | |
| O95168 | J08ab8 | NADH dehydrogenase | 2297.787 | 3270 | 2253.854 | 2607.423 | −2.79E−02 | 5.09E−01 | 3 | Unchanged High | |
| | | (ubiquino | | | | | | | | | |
| O95295 | J12ab8 | NADH dehydrogenase | 1909.371 | 2840 | 2888.281 | 2544.679 | 5.97E−01 | 5.71E−01 | 3 | Unchanged High | |
| | | (ubiquino | | | | | | | | | |
| O43920 | J18ab8 | NADH dehydrogenase | 6241.135 | 11500 | 8014.187 | 8594.08 | 3.61E−01 | 8.85E−01 | 3 | Unchanged High | |
| | | (ubiquino | | | | | | | | | |
| Q13765 | H09ab7 | nascent-polypeptide- | 2800.535 | 3390 | 3897.956 | 3363.371 | −4.77E−01 | 2.76E−01 | 3 | Unchanged High | |
| | | associated | | | | | | | | | |
| Q15843 | K13cd1 | neural precursor cell | 3449.484 | 4710 | 4139.56 | 4098.747 | 2.63E−01 | 4.48E−01 | 3 | Unchanged High | |
| | | expressed; | | | | | | | | | |
| Q9NX14 | C01gh3 | neuronal protein 17.3 | 3476.812 | 3150 | 5077.834 | 3902.8 | 5.46E−01 | −1.41E−01 | 3 | Unchanged High | |
| Q9Y2R6 | F18gh6 | NICE-3 protein | 1763.974 | 2250 | 2329.521 | 2113.511 | 4.01E−01 | 3.49E−01 | 3 | Unchanged High | |
| Q15868 | H13cd6 | Niemann-Pick disease; | 1516.592 | 3640 | 2828.255 | 2662.845 | 8.99E−01 | 1.26E+00 | 3 | Unchanged High | |
| | | type C2 | | | | | | | | | |
| O14597 | J21ef4 | non-functional folate | 2917.293 | 2170 | 3004.012 | 2697.108 | 4.23E−02 | −4.27E−01 | 3 | Unchanged High | |
| | | binding prot | | | | | | | | | |
| P15531 | E02ef5 | non-metastatic cells | 6653.515 | 6940 | 7836.897 | 7143.349 | 2.36E−01 | 6.07E−02 | 3 | Unchanged High | |
| | | 1; protein (N | | | | | | | | | |
| P22392 | L12cd2 | non-metastatic cells | 4827.494 | 5680 | 4836.596 | 5114.664 | 2.72E−03 | 2.35E−01 | 3 | Unchanged High | |
| | | 2; protein (N | | | | | | | | | |
| O00746 | D04eF7 | non-metastatic cells | 2211.058 | 2270 | 4300.604 | 2927.223 | 9.60E−01 | 3.80E−02 | 3 | Unchanged High | |
| | | 4; protein ex | | | | | | | | | |
| O75534 | B01cd4 | NRAS-related gene | 3478.657 3510 | 2737.425 | 3241.269 | −3.46E−01 | 1.20E−02 | 3 | Unchanged High | | |
| Q9H9A1 | C24gh6 | nuclear receptor | 5951.715 | 3290 | 2990.083 | 4076.544 | −9.93E−01 | −8.56E−01 | 3 | Unchanged High | |
| | | co-repressor/HD | | | | | | | | | |
| Q9NPE3 | K15ef4 | nucleolar protein | 3543.239 | 7130 | 3639.205 | 4771.656 | 3.86E−02 | 1.01E+00 | 3 | Unchanged High | |
| | | family A: memb | | | | | | | | | |
| P54368 | G10ef1 | ornithine decarboxy- | 7212.068 | 6880 | 7782.339 | 7292.818 | 1.10E−01 | −6.72E−02 | 3 | Unchanged High | |
| | | lase antizyme | | | | | | | | | |
| Q9UH52 | L23cd8 | over-expressed breast | 2335.527 | 2320 | 1884.264 | 2181.482 | −3.10E−01 | −6.73E−03 | 3 | Unchanged High | |
| | | tumor pro | | | | | | | | | |
| Q15070 | A09cd1 | oxidase (cytochrome c) | 2161.529 | 2500 | 1436.463 | 2031.796 | −5.90E−01 | 2.08E−01 | 3 | Unchanged High | |
| | | assembly | | | | | | | | | |
| Q9H230 | G14gh5 | p53-induced protein | 1823.554 | 3330 | 1443.914 | 2199.367 | −3.37E−01 | 8.69E−01 | 3 | Unchanged High | |
| | | PIGPC1 | | | | | | | | | |
| O60356 | M03cd8 | p8 protein (candidate | 1774.942 | 1940 | 2740.792 | 2150.631 | 6.27E−01 | 1.25E−01 | 3 | Unchanged High | |
| | | of metasta | | | | | | | | | |
| P78337 | M22ab8 | paired-like homeo- | 15428.96 | 13300 | 14490.63 | 14411.94 | −9.05E−02 | −2.12E−01 | 3 | Unchanged High | |
| | | domain transc | | | | | | | | | |
| Q9C086 | P20gh7 | PAP-1 binding protein | 2992.2 | 3610 | 5014.215 | 3872.969 | 7.45E−01 | 2.72E−01 | 3 | Unchanged High | |
| Q15165 | D06ef6 | paraoxonase 2 | 1325.972 | 3740 | 2122.119 | 2394.717 | 6.78E−01 | 1.49E+00 | 3 | Unchanged High | |
| P20962 | N13ab8 | parathymosin | 2345.869 | 2330 | 3489.616 | 2722.317 | 5.73E−01 | −8.88E−03 | 3 | Unchanged High | |
| P23284 | I17ab8 | peptidylprolyl | 5298.839 | 5860 | 8890.43 | 6683.166 | 7.47E−01 | 1.45E−01 | 3 | Unchanged High | |
| | | isomerase B (cyclo | | | | | | | | | |
| Q06830 | E18ab8 | peroxiredoxin 1 | 2973.862 | 2450 | 2056.112 | 2493.75 | −5.32E−01 | −2.79E−01 | 3 | Unchanged High | |
| P32119 | G04ef7 | peroxiredoxin 2 | 3141.718 | 3330 | 2921.471 | 3130.835 | −1.05E−01 | 8.37E−02 | 3 | Unchanged High | |
| P30048 | N09ab2 | peroxiredoxin 3 | 2420.936 | 2010 | 1671.796 | 2035.218 | −5.34E−01 | −2.66E−01 | 3 | Unchanged High | |
| Q13162 | N07ab2 | peroxiredoxin 4 | 2744.707 | 2080 | 3645.718 | 2822.139 | 4.10E−01 | −4.03E−01 | 3 | Unchanged High | |
| P30044 | P15cd7 | peroxiredoxin 5 | 2205.14 | 3820 | 1714.041 | 2581.24 | −3.63E−01 | 7.94E−01 | 3 | Unchanged High | |
| Q13492 | B13cd4 | phosphatidylinositol | 2688.917 | 2690 | 1861.508 | 2414.998 | −5.31E−01 | 3.03E−03 | 3 | Unchanged High | |
| | | binding clat | | | | | | | | | |
| P08237 | P02ab7 | phosphofructokinase: | 2790.708 | 1980 | 2829.743 | 2533.875 | 2.00E−02 | −4.94E−01 | 3 | Unchanged High | |
| | | muscle | | | | | | | | | |
| P00558 | P06ab7 | phosphoglycerate | 5186.346 | 5130 | 6341.68 | 5552.736 | 2.90E−01 | −1.57E−02 | 3 | Unchanged High | |
| | | kinase 1 | | | | | | | | | |
| P18669 | K14ab8 | phosphoglycerate | 1847.463 | 2730 | 2859.446 | 2477.75 | 6.30E−01 | 5.61E−01 | 3 | Unchanged High | |
| | | mutase 1 (brai | | | | | | | | | |
| Q14801 | B01cd8 | phosphoprotein enriched | 23286.56 | 17400 | 19729.36 | 20128.93 | −2.39E−01 | −4.23E−01 | 3 | Unchanged High | |
| | | in astrc | | | | | | | | | |
| O14832 | AO0cd1 | phytanoyl-CoA | 7333.104 | 7780 | 8341.917 | 7819.036 | 1.86E−01 | 8.57E−02 | 3 | Unchanged High | |
| | | hydroxylase (Ref | | | | | | | | | |
| P53801 | L01ef1 | pituitary tumor- | 4385.308 | 5330 | 4172.137 | 4627.881 | −7.19E−02 | 2.80E−61 | 3 | Unchanged High | |
| | | transforming 1 int | | | | | | | | | |
| Q9HB21 | E19gh5 | pleckstrin homology | 2318.08 | 3070 | 2907.381 | 2766.359 | 3.27E−01 | 4.07E−01 | 3 | Unchanged High | |
| | | domain-cont | | | | | | | | | |
| Q15365 | O09cd1 | poly(rC) binding | 4617.635 | 3130 | 3156.644 | 3635.431 | −5.49E−01 | −5.60E−01 | 3 | Unchanged High | |
| | | protein 1 | | | | | | | | | |
| P52433 | B17ab8 | polymerase (RNA) II | 2157.353 | 3780 | 3657.763 | 3198.285 | 7.62E−01 | 8.09E−01 | 3 | Unchanged High | |
| | | (DNA direct | | | | | | | | | |
| P52436 | L16gh1 | polymerase (RNA) II | 2161.398 | 2600 | 2709.495 | 2489.343 | 3.26E−01 | 2.65E−01 | 3 | Unchanged High | |
| | | (DNA direct | | | | | | | | | |
| Q03052 | B19ab8 | POU domain; class 3; | 3321.206 | 2650 | 2251.677 | 2740.913 | −5.61E−01 | −3.26E−01 | 3 | Unchanged High | |
| | | transcripti | | | | | | | | | |
| | K20gh5 PP1201 protein | 7127.293 | 6850 | 2688.636 | 5487.085 | −1.41E+00 | −1.01E−01 | 3 | Unchanged High | | |
| P40425 | G08ab8 | pre-B-cell leukemia | 2817.191 | 2650 | 3413.81 | 2958.976 | 2.77E−01 | −9.05E−02 | 3 | Unchanged High | |
| | | transcription | | | | | | | | | |
| Q99471 | K06ab8 | prefoldin 5 | 1827.777 | 2160 | 2637.334 | 2275.038 | 6.34E−01 | 2.41E−01 | 3 | Unchanged High | |
| Q9UHZ2 | E21ef8 | PRO1073 protein | 1872.208 | 3140 | 1690.101 | 2234.209 | −1.48E−01 | 7.46E−01 | 3 | Unchanged High | |
| P07737 | A21cd1 | profilin 1 | 29650.38 | 48900 | 35394.8 | 37970.06 | 2.55E−01 | 7.21E−01 | 3 | Unchanged High | |
| Q92740 | E03gh1 | prosaposin (variant | 4235.232 | 5370 | 4268.459 | 4622.898 | 1.13E−02 | 3.41E−01 | 3 | Unchanged High | |
| | | Gaucher dis | | | | | | | | | |
| P07478 | N02cd2 | protease: serine; | 77359.27 | 42600 | 51746.68 | 57240.67 | −5.80E−01 | −8.60E−01 | 3 | Unchanged High | |
| | | 2 (trypsin 2) | | | | | | | | | |
| Q9UL46 | N09ab8 | proteasome (prosome: | 3682.153 | 4550 | 2712.172 | 3648.995 | −4.41E−01 | 3.06E−01 | 3 | Unchanged High | |
| | | macropai | | | | | | | | | |
| P25786 | L09ef7 | proteasome (prosome: | 2270.079 | 2570 | 2265.763 | 2369.437 | −2.75E−03 | 1.80E−01 | 3 | Unchanged High | |
| | | macropai | | | | | | | | | |
| P20618 | J17ab8 | proteasome (prosome: | 2835.653 | 3590 | 2820.916 | 3082.003 | −7.52E−03 | 3.40E−01 | 3 | Unchanged High | |
| | | macropai | | | | | | | | | |
| P49720 | J21ab8 | proteasome (proseme: | 1714.767 | 2100 | 2435.426 | 2081.991 | 5.06E−01 | 2.89E−01 | 3 | Unchanged High | |
| | | macropai | | | | | | | | | |
| P28070 | J23ab8 | proteasome (prosome: | 2193.138 | 1760 | 2224.325 | 2058.212 | 2.04E−02 | −3.20E−01 | 3 | Unchanged High | |
| | | macropai | | | | | | | | | |
| Q99436 | L03ab8 | proteasome (prosome: | 4179.694 | 3450 | 3072.098 | 3566.368 | −4.44E−01 | −2.78E−01 | 3 | Unchanged High | |
| | | macropai | | | | | | | | | |
| P08129 | G01ef6 | protein phosphatase | 2543.089 | 2200 | 2276.462 | 2339.797 | −1.60E−01 | −2.09E−01 | 3 | Unchanged High | |
| | | 1; catalytic: | | | | | | | | | |
| O15355 | D07ab8 | protein phosphatase | 5896.408 | 4200 | 3717.65 | 4605.064 | −6.65E−01 | −4.69E−01 | 3 | Unchanged High | |
| | | 1G (formerl | | | | | | | | | |
| P30153 | A06ef3 | protein phosphatase | 3858.567 | 4100 | 2765.011 | 3572.89 | −4.81E−01 | 8.58E−02 | 3 | Unchanged High | |
| | | 2 (formerly: | | | | | | | | | |
| Q15249 | H13ef5 | prothymosin: alpha | 11382.44 | 14500 | 9974.587 | 11954.68 | −1.90E−01 | 3.50E−01 | 3 | Unchanged High | |
| | | (gene seque | | | | | | | | | |
| Q9N011 | G03gh7 | putative ATPase | 2774.724 | 2460 | 2271.593 | 2501.162 | −2.89E−01 | −1.75E−01 | 3 | Unchanged High | |
| P41567 | N06cd5 | putative translation | 7487.872 | 6600 | 8909.545 | 7688.779 | 2.51E−01 | −1.81E−01 | 3 | Unchanged High | |
| | | initiation fact | | | | | | | | | |
| Q15181 | L18gh1 | pyrophosphalase | 4914.897 | 4350 | 2667.605 | 3978.537 | −8.82E−01 | −1.75E−01 | 3 | Unchanged High | |
| | | (inorganic) | | | | | | | | | |
| P14786 | M24ab8 | pyruvate kinase; | 6305.222 | 8060 | 9000.504 | 7787.922 | 5.13E−01 | 3.54E−01 | 3 | Unchanged High | |
| | | muscle | | | | | | | | | |
| P57735 | G12gh4 | RAB25; member | 3343.628 | 4890 | 1366.379 | 3198.698 | −1.29E+00 | 5.47E−01 | 3 | Unchanged High | |
| | | RAS oncogene | | | | | | | | | |
| P54725 | E21cd1 | RAD23 homolog A | 2533.36 | 1790 | 1753.113 | 2026.126 | −5.31E−01 | −5.00E−01 | 3 | Unchanged High | |
| | | (S. cerevisiae) | | | | | | | | | |
| P54727 | M10ef6 | RAD23 homolog B | 4038.864 | 5410 | 4983.21 | 4809.132 | 3.03E−01 | 4.20E−01 | 3 | Unchanged High | |
| | | (S. cerevisiae) | | | | | | | | | |
| O00538 | P16cd5 | RAS guanyl releasing | 2418.688 | 2310 | 1906.199 | 2210.191 | −3.44E−01 | −6.90E−02 | 3 | Unchanged High | |
| | | protein 2 ( | | | | | | | | | |
| P06749 | C21gh1 | ras homolog gene | 2136.708 | 1940 | 3003.841 | 2358.668 | 4.91E−01 | −1.43E−01 | 3 | Unchanged High | |
| | | family; membe | | | | | | | | | |
| P08134 | B16ab2 | ras homolog gene | 4404.567 | 4490 | 2930.236 | 3943.029 | −5.88E−01 | 2.91E−02 | 3 | Unchanged High | |
| | | family: membe | | | | | | | | | |
| | P06gh8 | ras-like protein | 5451.542 | 6580 | 9066.798 | 7033.479 | 7.34E−01 | 2.72E−01 | 3 | Unchanged High | |
| | | VTS58635 | | | | | | | | | |
| P15154 | C01ef6 | ras-related C3 | 10849.13 | 9720 | 10044.19 | 10202.94 | −1.11E−01 | −1.59E−01 | 3 | Unchanged High | |
| | | botulinum toxin s | | | | | | | | | |
| Q15347 | B20cd6 | Ras-related GTP- | 1728.963 | 2380 | 2623.418 | 2244.978 | 6.02E−01 | 4.63E−01 | 3 | Unchanged High | |
| | | binding protein | | | | | | | | | |
| O95197 | A06cd6 | reticulon 3 | 3712.756 | 3420 | 3833.411 | 3654.06 | 4.61E−02 | −1.20E−01 | 3 | Unchanged High | |
| P09455 | F12ab8 | retinol binding | 2211.12 | 4480 | 2383.274 | 3023.27 | 1.08E−01 | 1.02E+00 | 3 | Unchanged High | |
| | | protein 1: cellular | | | | | | | | | |
| P52565 | K13ef6 | Rho GDP dissociation | 3166.04 | 3200 | 3141.434 | 3169.009 | −1.13E−02 | 1.52E−02 | 3 | Unchanged High | |
| | | inhibitor ( | | | | | | | | | |
| P27635 | N19cd2 | ribosomal protein | 13406.29 | 7100 | 11103.69 | 10536.25 | −2.72E−01 | −9.17E−01 | 3 | Unchanged High | |
| | | L10 | | | | | | | | | |
| P53025 | G22cd1 | ribosomal protein | 19374.73 | 15100 | 19939.26 | 18124.91 | 4.14E−02 | −3.63E−01 | 3 | Unchanged High | |
| | | L10a | | | | | | | | | |
| P39026 | D07cd1 | ribosomal protein | 14172.29 | 6760 | 10346.92 | 10427.07 | −4.54E−01 | −1.07E+00 | 3 | Unchanged High | |
| | | L11 | | | | | | | | | |
| P30050 | D09cd1 | ribosomal protein | 38376.69 | 24500 | 38669.34 | 33860.13 | 1.10E−02 | −6.45E−01 | 3 | Unchanged High | |
| | | L12 | | | | | | | | | |
| P40429 | A01ef1 | ribosomal protein | 23800.5 | 22200 | 28159.37 | 24735.85 | 2.43E−01 | −9.73E−02 | 3 | Unchanged High | |
| | | L13a | | | | | | | | | |
| P50914 | B18cd4 | ribosomal protein | 18507.36 | 14800 | 16108.18 | 16402.14 | −2.00E−01 | −3.43E−01 | 3 | Unchanged High | |
| | | L14 | | | | | | | | | |
| P39030 | C13cd3 | ribosomal protein | 8407.532 | 8100 | 10809.12 | 9104.589 | 3.62E−01 | −5.43E−02 | 3 | Unchanged High | |
| | | L15 | | | | | | | | | |
| P18621 | D19cd1 | ribosomal protein | 8779.141 | 8440 | 10004.78 | 9074.168 | 1.89E−01 | −5.71E−02 | 3 | Unchanged High | |
| | | L17 | | | | | | | | | |
| Q07020 | D13cd1 | ribosomal protein | 12168.43 | 10200 | 15619.17 | 12658.45 | 3.60E−01 | −2.56E−01 | 3 | Unchanged High | |
| | | L18 | | | | | | | | | |
| Q02543 | C15gh1 | ribosomal protein | 24976.59 | 27200 | 28170.19 | 26796.74 | 1.74E−01 | 1.25E−01 | 3 | Unchanged High | |
| | | L18a | | | | | | | | | |
| P14118 | D15cd1 | ribosomal protein | 6226.589 | 5530 | 5535.643 | 5762.718 | −1.70E−01 | −1.72E−01 | 3 | Unchanged High | |
| | | L19 | | | | | | | | | |
| P46778 | A13cd3 | ribosomal protein | 11156.59 | 11800 | 16742.21 | 13222.79 | 5.86E−01 | 7.72E−02 | 3 | Unchanged High | |
| | | L21 | | | | | | | | | |
| P35268 | D17cd1 | ribosomal protein | 16289.45 | 16200 | 18991.35 | 17168.94 | 2.21E−01 | −5.63E−03 | 3 | Unchanged High | |
| | | L22 | | | | | | | | | |
| P23131 | F11cd4 | ribosomal protein | 20972.93 | 20400 | 25729.93 | 22356.23 | 2.95E−01 | −4.24E−02 | 3 | Unchanged High | |
| | | L23 | | | | | | | | | |
| P29316 | G09cd3 | ribosomal protein | 6385.675 | 4840 | 5446.963 | 5556.302 | −2.29E−01 | −4.01E−01 | 3 | Unchanged High | |
| | | L23a | | | | | | | | | |
| P38663 | D21cd1 | ribosomal protein | 7805.803 | 6180 | 5479.195 | 6488.503 | −5.11E−01 | −3.37E−01 | 3 | Unchanged High | |
| | | L24 | | | | | | | | | |
| P08526 | F01cd1 | ribosomal protein | 12874.22 | 5280 | 10256.69 | 9469.252 | −3.28E−01 | −1.29E+00 | 3 | Unchanged High | |
| | | L27 | | | | | | | | | |
| P46776 | F05cd1 | ribosomal protein | 18046.37 | 17000 | 20949.85 | 18008.07 | 3.85E−01 | 8.57E−02 | 3 | Unchanged High | |
| | | L27a | | | | | | | | | |
| P46779 | F07cd1 | ribosomal protein | 40540.59 | 32400 | 45810.52 | 39577.25 | 1.76E−01 | −3.24E−01 | 3 | Unchanged High | |
| | | L28 | | | | | | | | | |
| P47914 | E23cd3 | ribosomal protein | 3346.473 | 1960 | 1947.028 | 2417.223 | −7.81E−01 | −7.73E−01 | 3 | Unchanged High | |
| | | L29 | | | | | | | | | |
| P39023 | B21cd1 | ribosomal protein | 13192.5 | 10500 | 13962.55 | 12535.38 | 8.18E−02 | −3.38E−01 | 3 | Unchanged High | |
| | | L3 | | | | | | | | | |
| P04645 | F03cd1 | ribosomal protein | 17693.61 | 12300 | 21530.83 | 17188.03 | 2.83E−01 | −5.20E−01 | 3 | Unchanged High | |
| | | L30 | | | | | | | | | |
| P12947 | F09cd1 | ribosomal protein | 9749.468 | 8980 | 12591.65 | 10441.63 | 3.69E−01 | −1.18E−01 | 3 | Unchanged High | |
| | | L31 | | | | | | | | | |
| P02433 | A15cd3 | ribosomal protein | 22774.44 | 24400 | 30742.77 | 25982.45 | 4.33E−01 | 1.01E−01 | 3 | Unchanged High | |
| | | L32 | | | | | | | | | |
| P49207 | F11cd1 | ribosomal protein | 13251.1 | 11500 | 13193.66 | 12661.96 | −6.27E−03 | −1.99E−01 | 3 | Unchanged High | |
| | | L34 | | | | | | | | | |
| P42766 | K16cd7 | ribosomal protein | 10454.27 | 10500 | 10655.65 | 10527.37 | 2.75E−02 | 2.47E−03 | 3 | Unchanged High | |
| | | L35 | | | | | | | | | |
| P18077 | F13cd1 | ribosomal protein | 14583.91 | 10700 | 8811.726 | 11356.13 | −7.27E−01 | −4.50E−01 | 3 | Unchanged High | |
| | | L35a | | | | | | | | | |
| P09896 | J06gh1 | ribosomal protein | 8395.644 | 8780 | 12052.18 | 9742.196 | 5.22E−01 | 6.44E−02 | 3 | Unchanged High | |
| | | L36a | | | | | | | | | |
| P09896 | A19cd3 | ribosomal protein | 5167.775 | 5750 | 4693.401 | 5203.379 | −1.39E−01 | 1.54E−01 | 3 | Unchanged High | |
| | | L36a-like | | | | | | | | | |
| P02403 | F15cd1 | ribosomal protein | 31895.4 | 45000 | 41301.54 | 39386.47 | 3.73E−01 | 4.95E−01 | 3 | Unchanged High | |
| | | L37 | | | | | | | | | |
| P12751 | F17cd1 | ribosomal protein | 18652.48 | 17300 | 19955.82 | 18651.98 | 9.74E−02 | −1.05E−01 | 3 | Unchanged High | |
| | | L37a | | | | | | | | | |
| P23411 | F19cd1 | ribosomal protein | 5149.82 | 5570 | 7560.21 | 6091.715 | 5.54E−01 | 1.12E−01 | 3 | Unchanged High | |
| | | L38 | | | | | | | | | |
| P02404 | A17cd3 | ribosomal protein | 14114.05 | 21300 | 22827.16 | 19427.01 | 6.94E−01 | 5.96E−01 | 3 | Unchanged High | |
| | | L39 | | | | | | | | | |
| P36578 | B23cd1 | ribosomal protein | 14367.47 | 10400 | 10448.85 | 11723.95 | −4.59E−01 | −4.72E−01 | 3 | Unchanged High | |
| | | L4 | | | | | | | | | |
| P28751 | L06gh1 | ribosomal protein | 35135.75 | 34300 | 40372.62 | 36614.61 | 2.00E−01 | −3.32E−02 | 3 | Unchanged High | |
| | | L41 | | | | | | | | | |
| P46777 | E21cd3 | ribosomal protein | 12002.66 | 8880 | 10351.69 | 10411.06 | −2.13E−01 | −4.35E−01 | 3 | Unchanged High | |
| | | L5 | | | | | | | | | |
| Q02878 | D01cd1 | ribosomal protein | 15861.7 | 7300 | 10119.01 | 11094.61 | −8.48E−01 | −1.12E+00 | 3 | Unchanged High | |
| | | L6 | | | | | | | | | |
| P18124 | D03cd1 | ribosomal protein | 21230.67 | 15900 | 19538.32 | 18885.03 | −1.20E−01 | −4.18E−01 | 3 | Unchanged High | |
| | | L7 | | | | | | | | | |
| P11518 | G11cd3 | ribosomal protein | 13385.5 | 8240 | 10586.53 | 10736.39 | −3.38E−01 | −7.00E−01 | 3 | Unchanged High | |
| | | L7a | | | | | | | | | |
| P25120 | D05cd1 | ribosomal protein | 23580.43 | 18300 | 24766.14 | 22208.81 | 7.08E−02 | −3.67E−01 | 3 | Unchanged High | |
| | | L8 | | | | | | | | | |
| P32969 | B19cd1 | ribosomal protein | 28853.42 | 32200 | 37288.41 | 32766.75 | 3.70E−01 | 1.56E−01 | 3 | Unchanged High | |
| | | L9 | | | | | | | | | |
| P46783 | H13cd1 | ribosomal protein | 7453.342 | 7660 | 7511.768 | 7540.695 | 1.13E−02 | 3.89E−02 | 3 | Unchanged High | |
| | | S10 | | | | | | | | | |
| P04643 | H15cd1 | ribosomal protein | 13429.85 | 15600 | 13623.52 | 14209.6 | 2.07E−02 | 2.14E−01 | 3 | Unchanged High | |
| | | S11 | | | | | | | | | |
| P25398 | H17cd1 | ribosomal protein | 16170.73 | 10600 | 12294.27 | 13018.69 | −3.95E−01 | −6.11E−01 | 3 | Unchanged High | |
| | | S12 | | | | | | | | | |
| Q02546 | H19cd1 | ribosomal protein | 13956.88 | 12600 | 15289.54 | 13946.74 | 1.32E−01 | −1.48E−01 | 3 | Unchanged High | |
| | | S13 | | | | | | | | | |
| P11174 | H21cd1 | ribosomal protein | 21322.73 | 19600 | 23334.6 | 21408.92 | 1.30E−01 | −1.24E−01 | 3 | Unchanged High | |
| | | S15 | | | | | | | | | |
| P39027 | H23cd1 | ribosomal protein | 16787.1 | 15100 | 21854.01 | 17912.92 | 3.81E−01 | −1.53E−01 | 3 | Unchanged High | |
| | | S15a | | | | | | | | | |
| P17008 | J01cd1 | ribosomal protein | 26604.5 | 15700 | 24481.55 | 22257.3 | −1.20E−01 | −7.62E−01 | 3 | Unchanged High | |
| | | S16 | | | | | | | | | |
| P08708 | J03cd1 | ribosomal protein | 13979.25 | 7990 | 15360.88 | 12443.09 | 1.36E−01 | −8.07E−01 | 3 | Unchanged High | |
| | | S17 | | | | | | | | | |
| P25232 | A17gh2 | ribosomal protein | 12558.18 | 12600 | 20093.07 | 15089.7 | 6.78E−01 | 6.84E−03 | 3 | Unchanged High | |
| | | S18 | | | | | | | | | |
| P17075 | J01cd1 | ribosomal protein | 21944.13 | 19900 | 31381.43 | 24400.98 | 5.16E−01 | −1.43E−01 | 3 | Unchanged High | |
| | | S20 | | | | | | | | | |
| P35285 | D24ef7 | ribosomal protein | 14046.7 | 9470 | 9023.678 | 10847.43 | −6.38E−01 | −5.69E−01 | 3 | Unchanged High | |
| | | S21 | | | | | | | | | |
| P39028 | J09cd1 | ribosomal protein | 12228.96 | 10900 | 14550.17 | 12545.6 | 2.51E−01 | −1.72E−01 | 3 | Unchanged High | |
| | | S23 | | | | | | | | | |
| P16632 | J11cd1 | ribosomal protein | 12913.37 | 13100 | 12135.49 | 12699.84 | −8.96E−02 | 1.53E−02 | 3 | Unchanged High | |
| | | S24 | | | | | | | | | |
| P25111 | J13cd1 | ribosomal protein | 6274.159 | 5630 | 5123.747 | 5677.337 | −2.92E−01 | −1.55E−01 | 3 | Unchanged High | |
| | | S25 | | | | | | | | | |
| P02383 | C01cd3 | ribosomal protein | 26138.26 | 30900 | 19440.2 | 25499.92 | −4.27E−01 | 2.42E−01 | 3 | Unchanged High | |
| | | S26 | | | | | | | | | |
| P42677 | G01cd3 | ribosomal protein | 17556.94 | 12800 | 20383.13 | 16927.4 | 2.15E−01 | −4.51E−01 | 3 | Unchanged High | |
| | | S27 (metallopa | | | | | | | | | |
| P14798 | B18cd1 | ribosomal protein | 3284.551 | 3180 | 2757.252 | 3073.17 | −2.52E−01 | −4.77E−02 | 3 | Unchanged High | |
| | | S27a | | | | | | | | | |
| P25112 | J15cd1 | ribosomal protein | 30384.24 | 32000 | 28529.21 | 30318.47 | −9.09E−02 | 7.66E−02 | 3 | Unchanged High | |
| | | S28 | | | | | | | | | |
| P30054 | J17cd1 | ribosomal protein | 18454.55 | 20900 | 25539.27 | 21630.2 | 4.69E−01 | 1.79E−01 | 3 | Unchanged High | |
| | | S29 | | | | | | | | | |
| P23396 | H03cd1 | ribosomal protein | 5913.682 | 2830 | 5523.561 | 4754.218 | −9.85E−02 | −1.07E+00 | 3 | Unchanged High | |
| | | S3 | | | | | | | | | |
| P49241 | O16ef5 | ribosomal protein | 7154.088 | 6200 | 8592.248 | 7316.687 | 2.64E−01 | −2.06E−01 | 3 | Unchanged High | |
| | | S3A | | | | | | | | | |
| P12750 | A21cd3 | ribosomal protein | 19821.66 | 16200 | 21778.05 | 19282.5 | 1.36E−01 | −2.87E−01 | 3 | Unchanged High | |
| | | S4; X-linked | | | | | | | | | |
| P22090 | A23cd3 | ribosomal protein | 2629.697 | 2330 | 2779.184 | 2580.924 | 7.98E−02 | −1.72E−01 | 3 | Unchanged High | |
| | | S4; Y-linked | | | | | | | | | |
| P46782 | H05cd1 | ribosomal protein | 15421.08 | 11000 | 17355.54 | 14598.96 | 1.70E−01 | −4.85E−01 | 3 | Unchanged High | |
| | | S5 | | | | | | | | | |
| P10660 | H07cd1 | ribosomal protein | 46408.18 | 26100 | 40579.46 | 37695.24 | −1.94E−01 | −8.30E−01 | 3 | Unchanged High | |
| | | S6 | | | | | | | | | |
| P23821 | H09cd1 | ribosomal protein | 18906.94 | 16200 | 14326.88 | 16494.24 | −4.00E−01 | −2.19E−01 | 3 | Unchanged High | |
| | | S7 | | | | | | | | | |
| P09058 | H11cd1 | ribosomal protein | 15941.2 | 12300 | 20997.59 | 16406.16 | 3.97E−01 | −3.76E−01 | 3 | Unchanged High | |
| | | S8 | | | | | | | | | |
| P46781 | A01cd5 | ribosomal protein | 6215.562 | 3930 | 5714.843 | 5285.193 | −1.21E−01 | −6.63E−01 | 3 | Unchanged High | |
| | | S9 | | | | | | | | | |
| P46781 | H12cd5 | ribosomal protein | 5527.416 | 9710 | 15295.98 | 11278.36 | 7.93E−01 1.38E−01 | 3 | Unchanged High | | |
| | | S9 | | | | | | | | | |
| P46781 | P12cd5 | ribosomal protein 9059.523 | 1790 | 5725.082 | 5525.883 | −6.62E−01 | −2.34E+00 | 3 | Unchanged High | | |
| | | S9 | | | | | | | | | |
| P05387 | H01cd1 | ribosomal protein; | 16611.14 | 12700 | 17775.72 | 15695.14 | 9.78E−02 | −3.87E−01 | 3 | Unchanged High | |
| | | large P2 | | | | | | | | | |
| P05388 | F21cd1 | ribosomal protein: | 17127.41 | 12400 | 19746.88 | 16430.54 | 2.05E−01 | −4.64E−01 | 3 | Unchanged High | |
| | | large; P0 | | | | | | | | | |
| P05386 | F23cd1 | ribosomal protein; | 2228.298 | 1880 | 3134.529 | 2413.78 | 4.92E−01 | −2.46E−01 | 3 | Unchanged High | |
| | | large; P1 | | | | | | | | | |
| Q9Y254 | C08ef3 | ring-box 1 | 2597.578 | 4260 | 3365.165 | 3408.892 | 3.74E−01 | 7.15E−01 | 3 | Unchanged High | |
| Q9NWJ8 | A23gh3 | roundabout homolog | 1969.602 | 3940 | 2093.279 | 2666.66 | 8.79E−02 | 9.99E−01 | 3 | Unchanged High | |
| | | 4; magic ro | | | | | | | | | |
| P08206 | D06cd1 | S100 calcium binding | 36485.73 | 49800 | 31745.47 | 40017.88 | −2.78E−01 | 3.72E−01 | 3 | Unchanged High | |
| | | protein A1 | | | | | | | | | |
| P31949 | E09cd3 | S100 calcium binding | 8890.594 | 11100 | 5408.269 | 8466.714 | −7.17E−01 | 3.20E−01 | 3 | Unchanged High | |
| | | protein A1 | | | | | | | | | |
| P06703 | P17gh6 | S100 calcium binding | 3403.146 | 4070 | 2066.478 | 3168.753 | −7.04E−01 | 2.60E−01 | 3 | Unchanged High | |
| | | protein A6 | | | | | | | | | |
| P55735 | J04ef4 | SEC 13-like 1 | 1895.152 | 1990 | 2362.541 | 2016.062 | 4.79E−01 | 2.32E−01 | 3 | Unchanged High | |
| | | (S. cerevisiae) | | | | | | | | | |
| P38384 | I06ef3 | Sec61 gamma | 5653.955 | 7820 | 7254.423 | 6908.432 | 3.60E−01 | 4.67E−01 | 3 | Unchanged High | |
| Q9NZJ3 | H06ef2 | selenoprotein T | 2923.813 | 2440 | 1325.491 | 2230.091 | −1.14E+00 | −2.60E−01 | 3 | Unchanged High | |
| O15532 | H16cd1 | selenoprotein W; 1 | 2878.843 | 2970 | 3918.44 | 3189.642 | 5.49E−01 | 1.50E−01 | 3 | Unchanged High | |
| Q13501 | J23cd4 | sequestosome 1 | 3214.395 | 3170 | 1164.17 | 2517.543 | −1.47E+00 | −1.82E−02 | 3 | Unchanged High | |
| P35237 | L18ab8 | serine (or cysteine) | 4203.074 | 8180 | 2936.72 | 5105.477 | −5.17E−01 | 9.60E−01 | 3 | Unchanged High | |
| | | proteinase in | | | | | | | | | |
| P05121 | P05ef7 | serine (or cysteine) | 4454.56 | 7280 | 9179.863 | 8972.284 | 1.04E+00 | 7.09E−01 | 3 | Unchanged High | |
| | | proteinase in | | | | | | | | | |
| Q00587 | C14cd7 | serum constituent | 2480.698 | 2660 | 2044.608 | 2393.46 | −2.79E−01 | 9.80E−02 | 3 | Unchanged High | |
| | | protein | | | | | | | | | |
| Q9H299 | P16gh7 | SH3 domain binding | 14473.6 | 17100 | 13798.02 | 15134.74 | −6.90E−02 | 2.43E−01 | 3 | Unchanged High | |
| | | glutamic aci | | | | | | | | | |
| P37108 | E21cd2 | signal recognition | 24326.27 | 35600 | 24319.59 | 28082.56 | −3.98E−04 | 5.49E−01 | 3 | Unchanged High | |
| | | particle 14kD ( | | | | | | | | | |
| P49458 | E19cd2 | signal recognition | 1874.967 | 2840 | 3141.422 | 2551.928 | 7.45E−01 | 4.93E−01 | 3 | Unchanged High | |
| | | particle 9kD | | | | | | | | | |
| P43308 | G15cd2 | signal sequence | 4209.147 | 4610 | 5876.451 | 4898.581 | 4.81E−01 | 1.31E−01 | 3 | Unchanged High | |
| | | receptor; beta (I | | | | | | | | | |
| P51571 | O15gh1 | signal sequence | 2062.113 | 2330 | 2654.968 | 2349.29 | 3.65E−01 | 1.77E−01 | 3 | Unchanged High | |
| | | receptor; delta ( | | | | | | | | | |
| O00422 | M17cd6 | sin3-associated | 2502.79 | 2610 | 2079.295 | 2398.002 | −2.87E−01 | 5.83E−02 | 3 | Unchanged High | |
| | | polypeplide; 18k | | | | | | | | | |
| O75918 | J10cd5 | small EDRK-rich | 5927.595 | 7010 | 7926.059 | 6955.245 | 4.19E−01 | 2.42E−01 | 3 | Unchanged High | |
| | | factor 2 | | | | | | | | | |
| P13500 | C15cd3 | small inducible | 17077.97 | 38100 | 8786.323 | 21330.26 | −9.59E−01 | 1.16E+00 | 3 | Unchanged High | |
| | | cytokine A2 (mon | | | | | | | | | |
| Q15356 | A09cd2 | small nuclear | 2743.428 | 2150 | 2819.855 | 2570.901 | 3.98E−02 | −3.52E−01 | 3 | Unchanged High | |
| | | ribonucleoprotein p | | | | | | | | | |
| P14648 | A13cd2 | small nuclear | 899.6242 | 2800 | 2362.597 | 2021.429 | 1.39E+00 | 1.64E+00 | 3 | Unchanged High | |
| | | ribonucleoprotein p | | | | | | | | | |
| P12236 | G01ef1 | solute carrier family | 7384.066 | 4760 | 8178.034 | 6107.995 | −2.58E−01 | −6.32E−01 | 3 | Unchanged High | |
| | | 25 (mitochor | | | | | | | | | |
| Q00325 | K22ab8 | solute carrier family | 5074.308 | 3750 | 3779.577 | 4200.626 | −4.25E−01 | −4.37E−01 | 3 | Unchanged High | |
| | | 25 (mitochor | | | | | | | | | |
| P32745 | N01cd1 | somatostatin receptor | 4102.668 | 4170 | 4748.989 | 4340.964 | 2.11E−01 | 2.39E−02 | 3 | Unchanged High | |
| | | 3 | | | | | | | | | |
| P30626 | E13cd2 | sorcin | 1895.592 | 2790 | 2280.297 | 2323.149 | 2.67E−01 | 5.59E−01 | 3 | Unchanged High | |
| Q01826 | F18ef8 | special AT-rich | 26449.32 | 41300 | 24225.61 | 30645.09 | −1.27E−01 | 6.42E−01 | 3 | Unchanged High | |
| | | sequence binding | | | | | | | | | |
| P21673 | D12cd1 | spermidine/spermine | 8349.882 | 16500 | 6013.659 | 10275.93 | −4.74E−01 | 9.80E−01 | 3 | Unchanged High | |
| | | N1-acetyltn | | | | | | | | | |
| P34991 | G05cd3 | S-phase kinase- | 3072.801 | 2990 | 2398.506 | 2820.643 | −3.59E−01 | −3.80E−02 | 3 | Unchanged High | |
| | | associated prote | | | | | | | | | |
| P23246 | B09cd2 | splicing factor | 2694.629 | 2460 | 3346.423 | 2834.056 | 3.12E−01 | −1.32E−01 | 3 | Unchanged High | |
| | | proline/glutamine | | | | | | | | | |
| P35716 | C07cd2 | SRY (sex determining | 5019.988 | 4860 | 2125.089 | 4001.241 | −1.24E+00 | −4.71E−02 | 3 | Unchanged High | |
| | | region Y)-I | | | | | | | | | |
| P35713 | E10ef4 | SRY (sex determining | 2575.791 | 3100 | 2024.386 | 2565.998 | −3.48E−01 | 2.66E−01 | 3 | Unchanged High | |
| | | region Y)-I | | | | | | | | | |
| Q06945 | C05cd2 | SRY (sex determining | 2299.474 | 2210 | 1638.067 | 2049.214 | −4.91E−01 | −5.59E−02 | 3 | Unchanged High | |
| | | region Y)-I | | | | | | | | | |
| P00441 | G20ef7 | superoxide dismutase | 4439.342 | 4930 | 3239.195 | 4202.954 | −4.55E−01 | 1.51E−01 | 3 | Unchanged High | |
| | | 1; soluble | | | | | | | | | |
| P50502 | I18cd2 | suppression of | 2307.599 | 2530 | 2358.117 | 2400.085 | 3.12E−02 | 1.35E−01 | 3 | Unchanged High | |
| | | tumorigenicity 13 | | | | | | | | | |
| Q9Y5Y6 | P20gh1 | suppression of | 2358.28 | 2700 | 1494.858 | 2183.041 | −6.58E−01 | 1.93E−01 | 3 | Unchanged High | |
| | | tumorigenicity 14 | | | | | | | | | |
| P17600 | J06cd2 | synapsin I | 1969.157 | 2220 | 2010.908 | 2067.417 | 3.03E−02 | 1.74E−01 | 3 | Unchanged High | |
| O43759 | G11cd5 | synaptogyrin 1 | 14516.6 | 10700 | 10060.61 | 11775.53 | −5.29E−01 | −4.33E−01 | 3 | Unchanged High | |
| Q12962 | P19cd2 | TAF 10 RNA | 5937.255 | 5080 | 3962.323 | 4992.528 | −5.83E−01 | −2.26E−01 | 3 | Unchanged High | |
| | | polymerase II; TATA | | | | | | | | | |
| Q15763 | B20cd2 | t-complex-associated- | 2022.997 | 2890 | 2885.017 | 2600.459 | 5.12E−01 | 5.16E−01 | 3 | Unchanged High | |
| | | testis-expr | | | | | | | | | |
| P10599 | B23cd4 | thioredoxin | 11525.16 | 11900 | 6440.37 | 9965.777 | −8.40E−01 | 5.00E−02 | 3 | Unchanged High | |
| P13472 | O02ef7 | thymosin; beta 10 | 46982.91 | 74300 | 54263.48 | 58508.68 | 2.08E−01 | 6.61E−01 | 3 | Unchanged High | |
| P01253 | L12gh1 | thymosin; beta 4; X | 23145.54 | 26009 | 16231.51 | 21783.42 | −5.12E−01 | 1.66E−01 | 3 | Unchanged High | |
| | | chromosome | | | | | | | | | |
| Q01085 | C02cd2 | TIA1 cytotoxic | 2250.456 | 1770 | 2917.342 | 2312.331 | 3.74E−01 | −3.47E−01 | 3 | Unchanged High | |
| | | granule-associate | | | | | | | | | |
| Q15370 | J12cd2 | transcription | 2616.595 | 3980 | 3919.327 | 3505.384 | 5.83E−01 | 6.05E−01 | 3 | Unchanged High | |
| | | elongation factor B | | | | | | | | | |
| O43680 | O11cd2 | transcription factor | 1789.295 | 1790 | 3044.118 | 2209.014 | 7.67E−01 | 3.49E−03 | 3 | Unchanged High | |
| | | 21 | | | | | | | | | |
| P21980 | O16cd2 | transglutaminase 2 | 3131.079 | 3490 | 1857.596 | 2827.774 | −7.53E−01 | 1.58E−01 | 3 | Unchanged High | |
| | | (C polypeptid | | | | | | | | | |
| P29401 | N15cd1 | transketolase | 3258.575 | 2040 | 2715.206 | 2671.504 | −2.63E−01 | −6.75E−01 | 3 | Unchanged High | |
| | | (Wernicke-Korsak | | | | | | | | | |
| O60739 | O01cd6 | translation factor | 2671.935 | 3400 | 3868.727 | 3315.099 | 5.34E−01 | 3.50E−01 | 3 | Unchanged High | |
| | | sui 1 homolog | | | | | | | | | |
| Q9NS69 | I02gh7 | translocase of outer | 2988.423 | 2450 | 3754.914 | 3062.809 | 3.29E−01 | −2.89E−01 | 3 | Unchanged High | |
| | | mitochondria | | | | | | | | | |
| P00938 | M24cd1 | triosephosphate | 5701.29 | 5820 | 8345.738 | 6622.243 | 5.50E−01 | 2.97E−02 | 3 | Unchanged High | |
| | | isomarase 1 | | | | | | | | | |
| P07226 | G10cd2 | tropomyosin 4 | 2636.258 | 2300 | 3755.882 | 2897.509 | 5.11E−01 | −1.97E−01 | 3 | Unchanged High | |
| P04687 | F09cd4 | tubulin; alpha 3 | 20220.03 | 14500 | 22303.88 | 18992.31 | 1.42E−01 | −4.84E−01 | 3 | Unchanged High | |
| P04687 | A01cd1 | tubulin; alpha; | 15258.81 | 7840 | 22478.65 | 15193.76 | 5.59E−01 | −9.80E−01 | 3 | Unchanged High | |
| | | ubiquitous | | | | | | | | | |
| Q13509 | E04cd6 | tubulin; beta; 4 | 2338.877 | 2990 | 1853.273 | 2394.89 | −3.36E−01 | 3.56E−01 | 3 | Unchanged High | |
| P04350 | F01gh6 | tubulin; beta; 5 | 7573.765 | 4430 | 3448.455 | 5149.498 | −1.14E+00 | −7.75E−01 | 3 | Unchanged High | |
| 075347 | O08cd2 | tubulin-specific | 3000.065 | 3900 | 3275.629 | 3390.719 | 1.27E−01 | 3.77E−01 | 3 | Unchanged High | |
| | | chaperone a | | | | | | | | | |
| 075509 | L10ef3 | tumor necrosis factor | 2245.338 | 4890 | 1881.231 | 3007.078 | −2.55E−01 | 1.12E+00 | 3 | Unchanged High | |
| | | receptor su | | | | | | | | | |
| P13693 | G16cd2 | tumor protein; | 39878.18 | 60800 | 48488.25 | 49731.11 | 2.81E−01 | 6.10E−01 | 3 | Unchanged High | |
| | | translationally-con | | | | | | | | | |
| P14625 | G24cd2 | tumor rejection | 2185.187 | 2060 | 4905.342 | 3049.346 | 1.17E+00 | −8.89E−02 | 3 | Unchanged High | |
| | | antigen (gp96) 1 | | | | | | | | | |
| P42655 | K04cd4 | tyrosine 3-mono- | 6584.565 | 5730 | 5558.974 | 5956.713 | −2.44E−01 | −2.01E−01 | 3 | Unchanged High | |
| | | oxygenase/trypto | | | | | | | | | |
| Q04917 | G06cd3 | tyrosine 3-mono- | 8621.322 | 8150 | 4562.366 | 7111.932 | −9.18E−01 | −8.07E−02 | 3 | Unchanged High | |
| | | oxygenase/trypto | | | | | | | | | |
| P29312 | A01cd4 | tyrosine 3-mono- | 5873.282 | 4670 | 2279.148 | 4275.506 | −1.37E+00 | −3.29E−01 | 3 | Unchanged High | |
| | | oxygenase/trypto | | | | | | | | | |
| P29312 | P12cd4 | tyrosine 3-mono- | 4562.964 | 1140 | 1520.2 | 2406.497 | −1.59E+00 | −2.01E+00 | 3 | Unchanged High | |
| | | oxygenase/trypto | | | | | | | | | |
| O14957 | G17cd7 | ubiquinol-cytochrome | 2728.765 | 3990 | 3332.422 | 3351.45 | 2.88E−01 | 5.49E−01 | 3 | Unchanged High | |
| | | c reductas | | | | | | | | | |
| P14793 | D01cd4 | ubiquitin A-52 residue | 3536.225 | 1730 | 3258.439 | 2840.732 | −1.18E−01 | −1.03E+00 | 3 | Unchanged High | |
| | | ribosomal | | | | | | | | | |
| P02248 | D22gh1 | ubiquitin B | 2753.942 | 4430 | 4152.235 | 3779.352 | 5.92E−01 | 6.86E−01 | 3 | Unchanged High | |
| 076069 | B12cd4 | ubiquitin-conjugating | 2001.262 | 1980 | 2223.574 | 2068.648 | 1.52E−01 | −1.46E−02 | 3 | Unchanged High | |
| | | enzyme E2 | | | | | | | | | |
| Q9BZL1 | B17gh7 | ubiquitin-like 5 | 4339.474 | 9100 | 5279.835 | 6239.513 | 2.83E−01 | 1.07E+00 | 3 | Unchanged High | |
| P08670 | C24cd3 | vimentin | 5940.876 | 8720 | 17783.4 | 10813.94 | 1.58E+00 | 5.53E−01 | 3 | Unchanged High | |
| P45880 | C18cd3 | voltage-dependent | 7005.537 | 7280 | 4758.179 | 6347.014 | −5.58E−01 | 5.49E−02 | 3 | Unchanged High | |
| | | anion channe | | | | | | | | | |
| Q9Y277 | C04cd4 | voltage-dependent | 1863.424 | 2210 | 2573.885 | 2216.75 | 4.26E−01 | 2.48E−01 | 3 | Unchanged High | |
| | | anion channe | | | | | | | | | |
| P17861 | O15cd4 | X-box binding protein | 3398.455 | 1600 | 2040.224 | 2347.333 | −7.36E−01 | −1.08E+00 | 3 | Unchanged High | |
| | | 1 | | | | | | | | | |
| O43670 | M04cd3 | zinc finger protein 207 | 3338.117 | 3170 | 3176.631 | 3226.643 | −7.15E−02 | −7.67E−02 | 3 | Unchanged High | |
| Q07352 | H21ef6 | zinc finger protein 36; | 6097.384 | 5750 | 4958.224 | 5601.518 | −2.98E−01 | −8.49E−02 | 3 | Unchanged High | |
| | | C3H type-I | | | | | | | | | |
| Q15942 | M10cd3 | zyxin | 2670.21 | 2070 | 2519.748 | 2367.987 | −2.86E−02 | −3.09E−01 | 3 | Unchanged High | |
| Q16659 | B17ef5 | mitogen-activated | 144.6425 | 141 | 100.0111 | 128.6073 | −5.32E−01 | −3.51E−02 | 1 | Unchanged Low | |
| | | protein kinase | | | | | | | | | |
| O75324 | B05cd3 | stannin | 131.7242 | 172 | 100.0655 | 134.7009 | −3.97E−01 | 3.88E−01 | 1 | Unchanged Low | |
| Q9BYP8 | E16gh8 | keratin associated | 176.4958 | 174 | 100.1011 | 150.0893 | −8.18E−01 | −2.33E−02 | 1 | Unchanged Low | |
| | | protein 17.1 | | | | | | | | | |
| P55291 | J01ab4 | cadherin 15; | 158.7215 | 128 | 100.3297 | 128.8583 | −6.62E−01 | −3.16E−01 | 1 | Unchanged Low | |
| | | M-cadherin (myotub | | | | | | | | | |
| P49768 | L14ab7 | presenilin 1 | 127.9653 | 124 | 100.3504 | 117.5823 | −3.51E−01 | −4.04E−02 | 1 | Unchanged Low | |
| | | (Alzheimer disease 3 | | | | | | | | | |
| P55107 | G10ab5 | growth differentiation | 114.7206 | 168 | 100.6326 | 127.9343 | −1.89E−01 | 5.54E−01 | 1 | Unchanged Low | |
| | | factor 10 | | | | | | | | | |
| P17082 | P18cd7 | related RAS viral | 433.219 | 176 | 100.8465 | 236.7776 | −2.10E+00 | −1.30E+00 | 1 | Unchanged Low | |
| | | (r-ras) oncoger | | | | | | | | | |
| P23634 | N08ab2 | ATPase; Ca++ | 179.3836 | 276 | 100.9041 | 185.4081 | −8.30E−01 | 6.21E−01 | 1 | Unchanged Low | |
| | | transporting; plas | | | | | | | | | |
| P78347 | J20ab5 | general transcription | 151.6834 | 103 | 100.9514 | 118.6963 | −5.67E−01 | −5.52E−01 | 1 | Unchanged Low | |
| | | factor II; i | | | | | | | | | |
| P09912 | F08ef7 | interferon; alpha- | 183.6419 | 1050 | 101.0749 | 444.3716 | −8.61E−01 | 2.51E+00 | 1 | Unchanged Low | |
| | | inducible protei | | | | | | | | | |
| Q9H2F5 | F13gh7 | enhancer of polycomb | 127.3938 | 115 | 101.3763 | 114.5773 | −3.30E−01 | −1.48E−01 | 1 | Unchanged Low | |
| | | 1 | | | | | | | | | |
| O14811 | M06cd7 | programmed cell | 152.3457 | 132 | 101.596 | 128.8019 | −5.85E−01 | −2.02E−01 | 1 | Unchanged Low | |
| | | death 10 | | | | | | | | | |
| O60942 | A17cd4 | RNA guanylyl- | 176.7836 | 97.1 | 101.6765 | 125.1735 | −7.98E−01 | −8.65E−01 | 1 | Unchanged Low | |
| | | transferase and 5′- | | | | | | | | | |
| Q9Y5Y3 | M18cd7 | G protein-coupled | 174.3335 | 161 | 101.8478 | 145.8365 | −7.75E−01 | −1.12E−01 | 1 | Unchanged Low | |
| | | receptor 45 | | | | | | | | | |
| P11908 | H15ab8 | phosphoribosyl | 166.1373 | 174 | 101.9334 | 147.2722 | −7.05E−01 | 6.46E−02 | 1 | Unchanged Low | |
| | | pyrophosphate s | | | | | | | | | |
| Q92851 | P18ab3 | caspase 10; apoptosis- | 135.919 | 107 | 101.9435 | 115.0308 | −4.15E−01 | −3.42E−01 | 1 | Unchanged Low | |
| | | related cy | | | | | | | | | |
| Q9BQE5 | M03gh7 | apolipoprotein L; 2 | 109.8196 | 148 | 102.0533 | 119.8218 | −1.06E−01 | 4.26E−01 | 1 | Unchanged Low | |
| P55327 | D05cd2 | tumor protein D52 | 136.2596 | 112 | 102.1111 | 116.9522 | −4.16E−01 | −2.77E−01 | 1 | Unchanged Low | |
| P28332 | K03ab2 | alcohol dehydrogenase | 142.4055 | 97.7 | 102.1575 | 114.0924 | −4.79E−01 | −5.43E−01 | 1 | Unchanged Low | |
| | | 6 (class | | | | | | | | | |
| O95861 | E02cd6 | 3′(2′); 5′- | 128.1251 | 116 | 102.1715 | 115.4153 | −3.27E−01 | −1.44E−01 | 1 | Unchanged Low | |
| | | bisphosphate nucleotide | | | | | | | | | |
| Q9NXJ5 | A10gh2 | hypothetical protein | 122.4219 | 151 | 102.3038 | 125.322 | −2.59E−01 | 3.05E−01 | 1 | Unchanged Low | |
| | | FLJ20208 | | | | | | | | | |
| Q15642 | K09gh1 | thyroid hormone | 120.9158 | 142 | 102.3082 | 121.6641 | −2.41E−01 | 2.30E−01 | 1 | Unchanged Low | |
| | | receptor interac | | | | | | | | | |
| O94997 | O09cd7 | decidual protein | 174.3087 | 140 | 102.3137 | 138.8592 | −7.69E−01 | −3.17E−01 | 1 | Unchanged Low | |
| | | induced by prog | | | | | | | | | |
| O95453 | G02ab8 | poly(A)-specific | 139.9344 | 105 | 102.3217 | 115.7147 | −4.52E−01 | −4.16E−01 | 1 | Unchanged Low | |
| | | ribonuclease (de | | | | | | | | | |
| Q9H9C5 | F01gh7 | KIAA1453 protein | 135.8299 | 126 | 102.4347 | 121.5247 | −4.07E−01 | −1.05E−01 | 1 | Unchanged Low | |
| P10451 | B13cd1 | secreted | 190.4604 | 76.7 | 102.5193 | 123.2388 | −8.94E−01 | −1.31E+00 | 1 | Unchanged Low | |
| | | phosphoprotein 1 (oste | | | | | | | | | |
| P53992 | B03cd5 | SEC24 related gene | 134.307 | 105 | 102.5437 | 113.9998 | −3.89E−01 | −3.53E−01 | 1 | Unchanged Low | |
| | | family; mem | | | | | | | | | |
| Q9NQW1 | F22gh6 | secretory pathway | 111.3643 | 158 | 102.6234 | 123.8833 | −1.18E−01 | 5.02E−01 | 1 | Unchanged Low | |
| | | component Se | | | | | | | | | |
| Q01449 | A17gh5 | myosin light chain 2a | 179.1031 | 170 | 103.0725 | 150.5811 | −7.97E−01 | −7.94E−02 | 1 | Unchanged Low | |
| Q9NX46 | K12gh2 | hypothetical protein | 109.5374 | 159 | 103.1083 | 123.8664 | −8.736−02 | 5.37E−01 | 1 | Unchanged Low | |
| | | FLJ20446 | | | | | | | | | |
| P53609 | A23cd1 | protein geranyl- | 140.0296 | 128 | 103.1248 | 123.8135 | −4.41E−01 | −1.26E−01 | 1 | Unchanged Low | |
| | | geranyltransferase | | | | | | | | | |
| Q15029 | J16cd4 | U5 snRNP-specific | 119.3284 | 126 | 103.1503 | 115.9932 | −2.10E−01 | 7.28E−02 | 1 | Unchanged Low | |
| | | protein; 116 | | | | | | | | | |
| P50458 | O23cd5 | LIM homeobox | 145.905 | 120 | 103.1971 | 122.9669 | −5.00E−01 | −2.84E−01 | 1 | Unchanged Low | |
| | | protein 2 | | | | | | | | | |
| P33764 | B24cd1 | S100 calcium binding | 150.5991 | 204 | 103.3783 | 152.5858 | −5.43E−01 | 4.36E−01 | 1 | Unchanged Low | |
| | | protein A3 | | | | | | | | | |
| Q9P289 | G03ef3 | Mst3 and SOK1- | 133.5225 | 116 | 103.4345 | 117.7841 | −3.68E−01 | −1.98E−01 | 1 | Unchanged Low | |
| | | related kinase | | | | | | | | | |
| P52429 | P17cd3 | diacylglycerol kinase; | 128.7238 | 123 | 103.5015 | 118.2956 | −3.15E−01 | −6.96E−02 | 1 | Unchanged Low | |
| | | epsilon (64 | | | | | | | | | |
| P54578 | H17cd5 | ubiquitin specific | 160.7495 | 108 | 103.6884 | 124.2132 | −6.33E−01 | −5.71E−01 | 1 | Unchanged Low | |
| | | protease 14 (tR | | | | | | | | | |
| Q13219 | E24ab8 | pregnancy-associated | 112.9052 | 154 | 103.7948 | 123.4254 | −1.21E−01 | 4.44E−01 | 1 | Unchanged Low | |
| | | plasma pr | | | | | | | | | |
| Q60547 | M12ab8 | GOP-macnose 4;6- | 147.2805 | 108 | 103.8004 | 119.8044 | −5.05E−01 | −4.43E−01 | 1 | Unchanged Low | |
| | | dehydratase | | | | | | | | | |
| P23297 | I17ef6 | S100 calcium binding | 145.2558 | 126 | 103.9033 | 124.9008 | −4.83E−01 | −2.10E−01 | 1 | Unchanged Low | |
| | | protein A1 | | | | | | | | | |
| Q9H5J8 | M01gh6 | hypothetical protein | 216.091 | 91.7 | 104.0432 | 137.2762 | −1.05E+00 | −1.24E+00 | 1 | Unchanged Low | |
| | | MGC5306 | | | | | | | | | |
| Q14493 | I08cd4 | stem-loop (histone) | 126.7755 | 155 | 104.1318 | 128.4702 | −2.84E−01 | 2.85E−01 | 1 | Unchanged Low | |
| | | binding prote | | | | | | | | | |
| Q9NZU0 | O16cd8 | fibronectin leucine | 284.9107 | 268 | 104.3033 | 219.1816 | −1.45E+00 | −8.65E−02 | 1 | Unchanged Low | |
| | | rich transmerr | | | | | | | | | |
| P80370 | B01ef7 | delta-like 1 homolog | 160.1421 | 80 | 104.328 | 114.8185 | −6.18E−01 | −1.00E+00 | 1 | Unchanged Low | |
| | | (Drosophila | | | | | | | | | |
| Q9NYV4 | C23ef3 | CDC2-related protein | 128.5947 | 107 | 104.3521 | 113.4542 | −3.01E−01 | −2.80E−01 | 1 | Unchanged Low | |
| | | kinase 7 | | | | | | | | | |
| P09016 | C23ef5 | homeo box D4 | 173.2254 | 192 | 104.3556 | 156.5829 | −7.31E−01 | 1.50E−01 | 1 | Unchanged Low | |
| Q13873 | A16ab3 | bone morphogenetic | 155.8113 | 86.8 | 104.4206 | 115.6624 | −5.77E−01 | −8.45E−01 | 1 | Unchanged Low | |
| | | protein rece | | | | | | | | | |
| O14878 | B22ef4 | inosine triphosphatase | 124.8814 | 120 | 104.4238 | 116.398 | −2.58E−01 | −5.89E−02 | 1 | Unchanged Low | |
| | | (nucleosi | | | | | | | | | |
| P52848 | J01ab6 | N-deacetylase/ | 166.2411 | 84.4 | 104.4555 | 118.3793 | −6.70E−01 | −9.77E−01 | 1 | Unchanged Low | |
| | | N-sulfotransterase | | | | | | | | | |
| Q14848 | A05cd5 | TNF receptor- | 124.2461 | 136 | 104.4604 | 121.4889 | −2.50E−01 | 1.27E−01 | 1 | Unchanged Low | |
| | | associated factor 4 | | | | | | | | | |
| O00204 | O04cd2 | sulfotransferase | 185.8106 | 117 | 104.4824 | 135.653 | −8.31E−01 | −6.71E−01 | 1 | Unchanged Low | |
| | | family; cytosolic | | | | | | | | | |
| P35610 | E13gh1 | sterol O-acyl- | 143.8417 | 88 | 104.5518 | 112.1404 | −4.60E−01 | −7.08E−01 | 1 | Unchanged Low | |
| | | transferase (acyl-Co | | | | | | | | | |
| Q04760 | L03ab5 | glyoxalase I | 132.6871 | 107 | 104.5782 | 114.7132 | −3.43E−01 | −3.12E−01 | 1 | Unchanged Low | |
| P51690 | H06ab2 | arylsulfatase E | 177.023 | 76.2 | 104.5865 | 119.2772 | −7.59E−01 | −1.22E+00 | 1 | Unchanged Low | |
| | | (chondrodysplasi | | | | | | | | | |
| P26012 | C11ef7 | integrin; beta 8 | 110.7396 | 170 | 104.6296 | 128.4899 | −8.19E−02 | 6.19E−01 | 1 | Unchanged Low | |
| Q14978 | K11cd5 | nucleolar and | 117.0048 | 131 | 104.685 | 117.4911 | −1.61E−01 | 1.61E−01 | 1 | Unchanged Low | |
| | | coiled-body phosp | | | | | | | | | |
| O00764 | D16cd3 | pyridoxal (pyridoxine; | 9160.2678 | 140 | 104.6915 | 135.073 | −6.14E−01 | −1.92E−01 | 1 | Unchanged Low | |
| | | vitamin B6 | | | | | | | | | |
| Q9Y6Q5 | P13cd5 | adaptor-related | 149.3929 | 107 | 104.1705 | 120.3004 | −5.12E−01 | −4.85E−01 | 1 | Unchanged Low | |
| | | protein complex | | | | | | | | | |
| O95350 | G04cd5 | Homer; neuronal | 137.2833 | 126 | 104.7829 | 122.6314 | −3.90E−01 | −1.26E−01 | 1 | Unchanged Low | |
| | | immediate early | | | | | | | | | |
| P49748 | C13ab2 | acyl-Coenzyme A | 121.151 | 141 | 104.7897 | 122.4441 | −2.09E−01 | 2.23E−01 | 1 | Unchanged Low | |
| | | dehydrogenas | | | | | | | | | |
| P08100 | O24cd1 | rhodopsin (opsin 2; | 125.3252 | 116 | 104.8101 | 115.4338 | −2.58E−01 | −1.10E−01 | 1 | Unchanged Low | |
| | | rod pigment) | | | | | | | | | |
| Q15477 | F24cd2 | superkiller viralicidic | 102.5551 | 186 | 104.8138 | 131.0636 | 3.14E−02 | 8.58E−01 | 1 | Unchanged Low | |
| | | activity 2-lik | | | | | | | | | |
| O43520 | I17cd1 | ATPase; Class I; | 199.1157 | 91.5 | 104.8252 | 131.8072 | −9.28E−01 | −1.12E+00 | 1 | Unchanged Low | |
| | | type 8B; memb | | | | | | | | | |
| Q13039 | P02ef4 | ATP-binding cassette; | 175.7841 | 80.5 | 104.8585 | 120.383 | −7.45E−01 | −1.13E+00 | 1 | Unchanged Low | |
| | | sub-family | | | | | | | | | |
| O15291 | L12cd1 | solute carrier family | 276.3689 | 176 | 104.904 | 185.7368 | −1.40E+00 | −6.52E−01 | 1 | Unchanged Low | |
| | | 7 (cationic a | | | | | | | | | |
| Q9NR71 | L23gh4 | mitochondrial | 108.9644 | 152 | 104.9361 | 122.091 | −5.43E−02 | 4.84E−01 | 1 | Unchanged Low | |
| | | ceramidase | | | | | | | | | |
| O60884 | H10cd4 | DnaJ (Hsp40) | 221.9835 | 239 | 104.9441 | 188.5763 | −1.08E+00 | 1.05E−01 | 1 | Unchanged Low | |
| | | homolog; subfamil | | | | | | | | | |
| Q9BYE0 | J17gh8 | hairy and enhancer | 128.0255 | 108 | 104.9542 | 113.8134 | −2.87E−01 | −2.39E−01 | 1 | Unchanged Low | |
| | | of split 7 (Dr | | | | | | | | | |
| Q9Y272 | L01ef2 | RAS; desamethasone- | 101.2051 | 166 | 104.9825 | 124.0354 | 5.29E−02 | 7.13E−01 | 1 | Unchanged Low | |
| | | induced 1 | | | | | | | | | |
| Q9BUR5 | M13gh6 | hypothetical protein | 78.2636 | 239 | 105.0137 | 140.65 | 4.24E−01 | 1.61E+00 | 1 | Unchanged Low | |
| | | MGC4825 | | | | | | | | | |
| O60859 | A13cd7 | neuropathy target | 123.9707 | 117 | 105.029 | 115.3513 | −2.39E−01 | −8.28E−02 | 1 | Unchanged Low | |
| | | esterase | | | | | | | | | |
| Q9GZU1 | A20gh4 | mucolipin 1 | 85.32301 | 243 | 105.1039 | 144.4516 | 3.01E−01 | 1.51E+00 | 1 | Unchanged Low | |
| P19878 | B15ab6 | neutrophil cytosolic | 310.5103 | 515 | 105.1041 | 310.3051 | −1.56E+00 | 7.31E−01 | 1 | Unchanged Low | |
| | | factor 2 (65ki | | | | | | | | | |
| Q13277 | K12cd2 | syntaxin 3A | 118.5375 | 185 | 105.2511 | 136.1937 | −1.72E−01 | 6.41E−01 | 1 | Unchanged Low | |
| | B04gh8 | reserved | 130.849 | 124 | 105.2594 | 120.0361 | −3.14E−01 | −7.76E−02 | 1 | Unchanged Low | |
| P32456 | N18ab4 | guanylate binding | 109.3786 | 142 | 105.2611 | 118.8515 | −5.54E−02 | 3.76E−01 | 1 | Unchanged Low | |
| | | protein 2; inter | | | | | | | | | |
| Q9H4M9 | E01cd7 | EH-domain containing | 149.0051 | 151 | 105.3893 | 134.9992 | −5.00E−01 | 1.54E−02 | 1 | Unchanged Low | |
| | | 1 | | | | | | | | | |
| Q9BTX7 | O19gh6 | chromosome 20 open | 115.9099 | 157 | 105.4128 | 126.203 | −1.37E−01 | 4.40E−01 | 1 | Unchanged Low | |
| | | reading fra | | | | | | | | | |
| Q9NVN2 | M02gh3 | solute carrier | 123.8178 | 114 | 105.466 | 114.478 | −2.31E−01 | −1.17E−01 | 1 | Unchanged Low | |
| | | family 4 (anion excl | | | | | | | | | |
| Q99758 | A17ab2 | ATP-binding cassette; | 238.456 | 145 | 105.5504 | 163.1362 | −1.18E+00 | −7.14E−01 | 1 | Unchanged Low | |
| | | sub-family | | | | | | | | | |
| P43694 | B06ef6 | GATA binding protein | 107.7539 | 161 | 105.5891 | 124.6996 | −2.93E−02 | 5.77E−01 | 1 | Unchanged Low | |
| | | 4 | | | | | | | | | |
| Q13825 | C07ab3 | AU RNA binding | 123.4494 | 140 | 105.8272 | 123.0884 | −2.25E−01 | 1.83E−01 | 1 | Unchanged Low | |
| | | protein/enoyl-C | | | | | | | | | |
| Q92550 | L23ef3 | ring finger protein 10 | 197.4603 | 112 | 105.7052 | 138.2365 | −9.02E−01 | −8.24E−01 | 1 | Unchanged Low | |
| Q9UJS0 | C12ef3 | solute carrier family | 119.1806 | 139 | 105.8216 | 121.1782 | −1.72E−01 | 2.17E−01 | 1 | Unchanged Low | |
| | | 25; member | | | | | | | | | |
| Q99611 | O05ef3 | seleriophosphate | 168.8742 | 128 | 105.8955 | 134.292 | −6.73E−01 | −3.99E−01 | 1 | Unchanged Low | |
| | | synthetase 2 | | | | | | | | | |
| P17275 | F08ab6 | jun B proto-oncogene | 135.9478 | 94.9 | 105.9141 | 112.2701 | −3.60E−01 | −5.18E−01 | 1 | Unchanged Low | |
| Q13477 | B09cd4 | mucosal vascular | 127.8352 | 110 | 105.9239 | 114.7415 | −2.71E−01 | −2.11E−01 | 1 | Unchanged Low | |
| | | acddressin cell | | | | | | | | | |
| Q14692 | D09gh1 | KIAA0187 gene | 114.5267 | 131 | 105.9487 | 117.1898 | −1.12E−01 | 1.95E−01 | 1 | Unchanged Low | |
| | | product | | | | | | | | | |
| P49908 | D13cd2 | selenoprotein P; | 140.6083 | 104 | 105.9794 | 116.7316 | −4.08E−01 | −4.41E−01 | 1 | Unchanged Low | |
| | | plasma; 1 | | | | | | | | | |
| P54803 | M24ab3 | galactosylceramidase | 130.3854 | 105 | 105.9866 | 113.8221 | −2.99E−01 | −3.11E−01 | 1 | Unchanged Low | |
| | | (Krabbe di | | | | | | | | | |
| P50991 | H09cd6 | chaperonin containing | 161.4396 | 148 | 106.0337 | 138.5613 | −6.06E−01 | −1.23E−01 | 1 | Unchanged Low | |
| | | TCP1: sut | | | | | | | | | |
| P35548 | K11ab7 | msh homeo box | 182.4309 | 124 | 106.057 | 137.5011 | −7.83E−01 | −5.57E−01 | 1 | Unchanged Low | |
| | | homolog 2 (Dros | | | | | | | | | |
| P11309 | M14ab8 | pim-1 oncogene | 136.3951 | 99 | 106.0757 | 113.8227 | −3.63E−01 | −4.62E−01 | 1 | Unchanged Low | |
| O75570 | A03cd5 | mitochondrial | 128.9238 | 118 | 106.1208 | 117.597 | −2.81E−01 | −1.31E−01 | 1 | Unchanged Low | |
| | | translational releas | | | | | | | | | |
| Q9NY61 | O01ef3 | apoptosis antagonizing | 120.3605 | 124 | 106.1404 | 118.8527 | −1.81E−01 | 4.36E−02 | 1 | Unchanged Low | |
| | | transcrip | | | | | | | | | |
| Q9BTY8 | D01gh7 | hypothetical protein | 139.5304 | 92.9 | 106.2168 | 112.876 | −3.94E−01 | −5.87E−01 | 1 | Unchanged Low | |
| | | MGC4342 | | | | | | | | | |
| Q9NWS2 | F09gh2 | hypothetical protein | 183.0823 | 140 | 106.2384 | 143.0354 | −7.85E−01 | −3.8gE−01 | 1 | Unchanged Low | |
| | | FLJ20640 | | | | | | | | | |
| O00180 | H06ab6 | potassium channel: | 129.4915 | 122 | 106.2703 | 119.3824 | −2.85E−01 | −8.14E−02 | 1 | Unchanged Low | |
| | | subfamily K; | | | | | | | | | |
| O15382 | I05ab3 | branched chain | 136.8391 | 95.5 | 106.3058 | 112.8868 | −3.64E−01 | −5.19E−01 | 1 | Unchanged Low | |
| | | aminotransferase | | | | | | | | | |
| Q9Y2Z1 | D05ef2 | CGI-01 protein | 156.4563 | 196 | 106.3369 | 153.067 | −5.57E−01 | 3.28E−01 | 1 | Unchanged Low | |
| Q9Y315 | E18ef2 | CGI-26 protein | 135.7793 | 111 | 106.6662 | 117.8557 | −3.48E−01 | −2.89E−01 | 1 | Unchanged Low | |
| Q9Y592 | P20ef1 | NY-REN-58 antigen | 118.6541 | 121 | 106.7049 | 115.3792 | −1.53E−01 | 2.56E−02 | 1 | Unchanged Low | |
| Q9NYT0 | D20ef3 | pleckstrin 2 | 114.999 | 153 | 106.7508 | 124.8364 | −1.07E−01 | 4.10E−01 | 1 | Unchanged Low | |
| | | (mouse) homolog | | | | | | | | | |
| Q9Y343 | M10ef4 | SBBI31 protein | 123.0625 | 120 | 106.8362 | 116.6438 | −2.04E−01 | −3.60E−02 | 1 | Unchanged Low | |
| Q9BV14 | G09gh6 | hypothetical protein | 140.5988 | 123 | 106.8523 | 123.6298 | −3.96E−01 | −1.88E−01 | 1 | Unchanged Low | |
| | | MGC3162 | | | | | | | | | |
| P30622 | B20cd1 | restin (Reed- | 116.8074 | 136 | 107.0013 | 119.7985 | −1.27E−01 | 2.15E−01 | 1 | Unchanged Low | |
| | | Steinberg cell-expre | | | | | | | | | |
| Q08345 | F08ef5 | discoidin domain | 266.6468 | 264 | 107.136 | 212.6497 | −1.32E+00 | −1.35E−02 | 1 | Unchanged Low | |
| | | receptor family; | | | | | | | | | |
| P33176 | C16ab7 | kinesin family | 123.5679 | 113 | 107.2314 | 114.5075 | −2.05E−01 | −1.33E−01 | 1 | Unchanged Low | |
| | | member 5B | | | | | | | | | |
| P55273 | D14ab4 | cyclin-dependent | 99.64375 | 174 | 107.4234 | 126.8971 | 1.08E−01 | 8.01E−01 | 1 | Unchanged Low | |
| | | kinase inhibitor | | | | | | | | | |
| Q9NW92 | E10gh3 | hypothetical protein | 177.1237 | 166 | 107.454 | 150.3287 | −7.21E−01 | −9.00E−02 | 1 | Unchanged Low | |
| | | FLJ10209 | | | | | | | | | |
| Q9NZC3 | K03ef3 | membrane interacting | 105.8303 | 152 | 107.465 | 121.8015 | 2.21E−02 | 5.23E−01 | 1 | Unchanged Low | |
| | | protein of | | | | | | | | | |
| P15172 | M19ab7 | myogenic factor 3 | 132.7839 | 110 | 107.4694 | 116.7347 | −3.05E−01 | −2.72E−01 | 1 | Unchanged Low | |
| P27144 | A06ab2 | adenylate kinase 3 | 118.5733 | 124 | 107.5998 | 116.7128 | −1.40E−01 | 6.42E−02 | 1 | Unchanged Low | |
| O60508 | A06ef2 | pre-mRNA splicing | 150.1968 | 130 | 107.6187 | 129.259 | −4.81E−01 | −2.09E−01 | 1 | Unchanged Low | |
| | | factor 17 | | | | | | | | | |
| Q14012 | B08cd3 | calcium/calmodulin- | 113.9231 | 135 | 107.6204 | 118.8965 | −8.21E−02 | 2.46E−01 | 1 | Unchanged Low | |
| | | dependent p | | | | | | | | | |
| P98179 | G12cd1 | RNA binding motif | 140.1356 | 93.2 | 107.7553 | 113.6891 | −3.79E−01 | −5.89E−01 | 1 | Unchanged Low | |
| | | protein 3 | | | | | | | | | |
| Q13223 | E10ab5 | BRF1 homolog: | 150.0469 | 115 | 107.7567 | 124.3969 | −4.78E−01 | −3.79E−01 | 1 | Unchanged Low | |
| | | subunit of RNA | | | | | | | | | |
| P55198 | N09ab7 | myeloid/lymphoid or | 180.7113 | 122 | 107.7762 | 136.7788 | −7.46E−01 | −5.69E−01 | 1 | Unchanged Low | |
| | | mixed-linea | | | | | | | | | |
| P46459 | G02ef1 | N-ethylmaleimide- | 138.9363 | 95.7 | 107.8044 | 114.1635 | −3.66E−01 | −5.37E−01 | 1 | Unchanged Low | |
| | | sensitive facto | | | | | | | | | |
| P34969 | I2lef1 | 5-hydroxytryptamine | 218.7666 | 181 | 107.8181 | 169.3507 | −1.02E+00 | −2.70E−01 | 1 | Unchanged Low | |
| | | (serotonin) | | | | | | | | | |
| Q14667 | K02gh1 | KIAA0100 gene | 105.9471 | 222 | 107.8612 | 145.1883 | 2.58E−02 | 1.07E+00 | 1 | Unchanged Low | |
| | | product | | | | | | | | | |
| Q92830 | J14cd8 | GCN5 general control | 129.4527 | 106 | 107.863 | 114.5758 | −2.63E−01 | −2.83E−01 | 1 | Unchanged Low | |
| | | of amino- | | | | | | | | | |
| | P14cd2 | RNA binding motif | 112.3917 | 153 | 107.9125 | 124.4395 | −5.87E−02 | 4.45E−01 | 1 | Unchanged Low | |
| | | protein; Y chr | | | | | | | | | |
| O43252 | J01cd5 | 3′-phosphoadenosine | 194.4946 | 188 | 107.9154 | 163.4169 | −8.50E−01 | −5.02E−02 | 1 | Unchanged Low | |
| | | 5′-phospho | | | | | | | | | |
| Q9NS93 | G09ef3 | seven transmembrane | 138.0932 | 237 | 107.9483 | 160.8518 | −3.55E−01 | 7.76E−01 | 1 | Unchanged Low | |
| | | protein T | | | | | | | | | |
| O60616 | E20ab6 | core-binding factor; | 489.0924 | 48.1 | 107.9969 | 215.0608 | −2.18E+00 | −3.35E+00 | 1 | Unchanged Low | |
| | | runt domain; | | | | | | | | | |
| P02261 | D19cd4 | H2A histone family: | 145.0821 | 84.1 | 108.023 | 112.4051 | −4.26E−01 | −7.87E−01 | 1 | Unchanged Low | |
| | | member C | | | | | | | | | |
| Q14701 | F18ef7 | DNA cross-link repair | 119.2486 | 307 | 108.208 | 178.2467 | −1.40E−01 | 1.37E+00 | 1 | Unchanged Low | |
| | | 1A (PSO2 | | | | | | | | | |
| Q9P025 | A04ef8 | HSPC135 protein | 160.3109 | 135 | 108.2493 | 134.6437 | −5.67E−01 | −2.44E−01 | 1 | Unchanged Low | |
| P06702 | O12ef7 | S100 calcium binding | 144.7319 | 163 | 108.2576 | 138.5985 | −4.19E−01 | 1.70E−01 | 1 | Unchanged Low | |
| | | protein A9 | | | | | | | | | |
| Q9Y5Q9 | P05cd7 | general transcription | 138.644 | 220 | 108.3524 | 155.7377 | −3.56E−01 | 6.68E−01 | 1 | Unchanged Low | |
| | | factor IIIC: | | | | | | | | | |
| P19075 | O20cd2 | transmerebrane 4 | 129.5468 | 103 | 108.6355 | 113.7491 | −2.54E−01 | −3.30E−01 | 1 | Unchanged Low | |
| | | superfamily m | | | | | | | | | |
| O60905 | B16cd4 | sperm associated | 110.7683 | 139 | 108.6913 | 119.4574 | −2.73E−02 | 3.27E−01 | 1 | Unchanged Low | |
| | | antigen 9 | | | | | | | | | |
| Q13322 | F24ef5 | growth factor receptor- | 120.8593 | 115 | 108.7182 | 114.7351 | −1.53E−01 | −7.64E−02 | 1 | Unchanged Low | |
| | | bound pro | | | | | | | | | |
| Q9UQ09 | L01gh1 | KIAA0470 gene | 190.3667 | 103 | 108.9005 | 134.0152 | −8.06E−01 | −8.89E−01 | 1 | Unchanged Low | |
| | | product | | | | | | | | | |
| P57053 | J20gh6 | H2B histone family: | 113.6057 | 199 | 108.9238 | 140.5964 | −6.07E−02 | 8.11E−01 | 1 | Unchanged Low | |
| | | member S | | | | | | | | | |
| Q92876 | J03ab8 | kallikrein 6 (neurosin: | 197.2979 | 130 | 108.9308 | 145.5667 | −8.57E−01 | −5.97E−01 | 1 | Unchanged Low | |
| | | zyme) | | | | | | | | | |
| P09960 | F21ab6 | leukotriene A4 | 96.36818 | 185 | 109.0526 | 130.2822 | 1.78E−01 | 9.44E−01 | 1 | Unchanged Low | |
| | | hydrolase | | | | | | | | | |
| Q9HBH1 | O02gh5 | peptide deformylase- | 138.0653 | 91 | 109.0612 | 112.7249 | −3.40E−01 | −8.01E−01 | 1 | Unchanged Low | |
| | | like protein | | | | | | | | | |
| P55082 | L15ab7 | microfibrillar- | 121.4467 | 124 | 109.134 | 118.1545 | −1.54E−01 | 2.87E−02 | 1 | Unchanged Low | |
| | | associated protein 3 | | | | | | | | | |
| Q9NVH6 | B01gh3 | trimethyllysine | 174.7559 | 81.6 | 109.1442 | 121.828 | −6.79E−01 | −1.10E+00 | 1 | Unchanged Low | |
| | | hydroxylase: epsil | | | | | | | | | |
| O95249 | K10cd5 | golgi SNAP receptor | 107.5878 | 143 | 109.1657 | 119.983 | 2.10E−02 | 4.12E−01 | 1 | Unchanged Low | |
| | | complex me | | | | | | | | | |
| Q13891 | M15ab6 | basic transcription | 126.6543 | 120 | 109.1942 | 118.5076 | −2.14E−01 | −8.18E−02 | 1 | Unchanged Low | |
| | | factor 3: like 2 | | | | | | | | | |
| Q9BZM5 | H05gh7 | UL16 binding | 133.6731 | 245 | 109.2193 | 162.5485 | −2.91E−01 | 8.73E−01 | 1 | Unchanged Low | |
| | | protein 2 | | | | | | | | | |
| O94913 | O20ef2 | PCF11p homolog | 205.6752 | 80.3 | 109.2409 | 131.7517 | 9.13E−01 | −1.36E+00 | 1 | Unchanged Low | |
| Q12987 | E03ef4 | acidic 82kDa | 132.1136 | 117 | 109.4318 | 119.3623 | −2.72E−01 | −1.81E−01 | 1 | Unchanged Low | |
| | | protein mRNA | | | | | | | | | |
| Q92499 | D05ab5 | DEAD/H (Asp-Glu- | 176.6983 | 124 | 109.4356 | 136.6656 | −6.91E−01 | −5.13E−01 | 1 | Unchanged Low | |
| | | Ala-Asp/His) t | | | | | | | | | |
| Q13686 | B19ab2 | alkylation repair: | 105.5169 | 143 | 109.4797 | 119.4385 | 5.32E−02 | 4.42E−01 | 1 | Unchanged Low | |
| | | alkB homolog | | | | | | | | | |
| P43250 | I08ab4 | G protein-coupled | 120.3579 | 113 | 109.4844 | 114.2814 | −1.37E−01 | −9.10E−02 | 1 | Unchanged Low | |
| | | receptor kinas | | | | | | | | | |
| Q9UMX0 | L03ef4 | ubiquilin 1 | 122.0111 | 141 | 109.5522 | 124.0398 | −1.55E−01 | 2.04E−01 | 1 | Unchanged Low | |
| O94905 | G12cd7 | chromosome 8 open | 127.8659 | 107 | 109.6202 | 114.8261 | −2.22E−01 | −2.57E−01 | 1 | Unchanged Low | |
| | | reading frar | | | | | | | | | |
| Q9UHK0 | K01cd8 | nuclear fragile X | 157.4757 | 87.6 | 109.6296 | 118.2396 | −5.22E−01 | −8.46E−01 | 1 | Unchanged Low | |
| | | mental retardati | | | | | | | | | |
| P25189 | D01ab6 | myelin protein zero | 110.5502 | 132 | 109.6859 | 117.3088 | −1.13E−02 | 2.52E−01 | 1 | Unchanged Low | |
| | | (Charcot-Ma | | | | | | | | | |
| Q10567 | M11ab2 | adaptor-related | 208.9283 | 187 | 109.8204 | 168.4738 | −9.28E−01 | −1.62E−01 | 1 | Unchanged Low | |
| | | protein complex | | | | | | | | | |
| O15230 | D18cd8 | laminin: alpha 5 | 111.864 | 141 | 109.8469 | 120.9363 | −2.63E−02 | 3.35E−01 | 1 | Unchanged Low | |
| P52294 | J10ab6 | karyopherin alpha 1 | 178.7513 | 169 | 109.9831 | 152.7331 | −7.01E−01 | −7.70E−02 | 1 | Unchanged Low | |
| | | (importin alp | | | | | | | | | |
| O60668 | M03cd2 | TAF2 RNA polymerase | 123.6244 | 111 | 110.0571 | 114.8794 | −1.68E−01 | −1.56E−01 | 1 | Unchanged Low | |
| | | II: TATA | | | | | | | | | |
| P11686 | J02cd1 | surfactant: | 136.0267 | 117 | 110.0847 | 121.0017 | −3.05E−01 | −2.19E−01 | 1 | Unchanged Low | |
| | | pulmonary-associate | | | | | | | | | |
| Q9NWB6 | P03gh2 | hypothetical protein | 174.7789 | 75 | 110.1306 | 119.9579 | −6.66E−01 | −1.22E+00 | 1 | Unchanged Low | |
| | | FLJ10154 | | | | | | | | | |
| Q9H028 | E22gh8 | histone deacatylase 10 | 107.5034 | 138 | 110.1585 | 118.4626 | 3.52E−02 | 3.57E−01 | 1 | Unchanged Low | |
| Q9UMZ2 | O22cd7 | AP1 gamma subunit | 120.2821 | 119 | 110.2219 | 116.5628 | −1.26E−01 | −1.32E−02 | 1 | Unchanged Low | |
| | | binding prot | | | | | | | | | |
| P24752 | C15ab2 | acetyl-Coenzyme A | 216.1409 | 114 | 110.2562 | 146.8035 | −9.71E−01 | −9.23E−01 | 1 | Unchanged Low | |
| | | acetyltransfe | | | | | | | | | |
| P11230 | B06ab3 | cholinergic receptor: | 106.1774 | 151 | 110.2999 | 122.607 | 5.50E−02 | 5.11E−01 | 1 | Unchanged Low | |
| | | nicotinic: be | | | | | | | | | |
| O14977 | O18ef2 | ornithine decarboxy- | 119.9172 | 129 | 110.3 | 119.7626 | −1.21E−01 | 1.06E−01 | 1 | Unchanged Low | |
| | | lase antizym | | | | | | | | | |
| | E15ef5 | T cell receptor | 139.7091 | 844 | 110.3287 | 364.6946 | −3.41E−01 | 2.59E+00 | 1 | Unchanged Low | |
| | | beta locus | | | | | | | | | |
| P56937 | N06ef2 | hydroxysteroid | 227.1137 | 74.5 | 110.3428 | 137.3284 | −1.04E+00 | −1.61E+00 | 1 | Unchanged Low | |
| | | (17-beta) dehydr | | | | | | | | | |
| O75299 | B21ab7 | kinesin family | 113.15 | 128 | 110.3535 | 117.1803 | −3.61E−02 | 1.78E−01 | 1 | Unchanged Low | |
| | | member C3 | | | | | | | | | |
| P16219 | C09ab2 | acyl-Coenzyme A | 109.4688 | 136 | 110.3636 | 118.89 | 1.17E−02 | 3.16E−01 | 1 | Unchanged Low | |
| | | dehydrogenas | | | | | | | | | |
| Q9NZ48 | J20gh3 | uncharacterized | 112.5665 | 189 | 110.3995 | 137.2614 | −2.80E−02 | 7.46E−01 | 1 | Unchanged Low | |
| | | hematopoietic s | | | | | | | | | |
| Q9P163 | L10gh4 | hypothetical protein | 101.1317 | 159 | 110.4184 | 123.4514 | 1.27E−01 | 6.51E−01 | 1 | Unchanged Low | |
| | | PRO2521 | | | | | | | | | |
| Q9BVT8 | I07gh8 | hypothetical protein | 135.5302 | 137 | 110.5044 | 127.7419 | −2.95E−01 | 1.76E−02 | 1 | Unchanged Low | |
| | | MGC5442 | | | | | | | | | |
| Q99933 | E02ef6 | BCL2-associated | 119.7332 | 115 | 110.533 | 115.0469 | −1.15E−01 | −5.98E−02 | 1 | Unchanged Low | |
| | | athanogene | | | | | | | | | |
| O14645 | M12cd3 | dynein: axonemal; | 164.1806 | 207 | 110.5493 | 160.4204 | −5.71E−01 | 3.31E−01 | 1 | Unchanged Low | |
| | | light intermedi | | | | | | | | | |
| Q9BRR9 | E01gh8 | Rho GTPase activating | 120.3646 | 115 | 110.5606 | 115.1711 | −1.23E−01 | −7.10E−02 | 1 | Unchanged Low | |
| | | protein 9 | | | | | | | | | |
| Q9Y3E7 | J19ef2 | CGI-149 protein | 158.4397 | 202 | 110.6074 | 157.1807 | −5.18E−01 | 3.54E−01 | 1 | Unchanged Low | |
| P46976 | C09ab6 | glycogenin | 127.6502 | 117 | 110.6815 | 118.5706 | −2.06E−01 | −1.21E−01 | 1 | Unchanged Low | |
| Q75928 | C01cd5 | Protein inhibitor of | 95.84393 | 173 | 110.7286 | 127.4786 | 1.64E−01 | 8.06E−01 | 1 | Unchanged Low | |
| | | activated STA | | | | | | | | | |
| Q9NX63 | I18gh2 | hypothetical protein | 123.3725 | 159 | 110.8068 | 131.146 | −1.55E−01 | 3.68E−01 | 1 | Unchanged Low | |
| | | FLJ20420 | | | | | | | | | |
| Q9UBS0 | I20cd2 | ribosomal protein S6 | 186.953 | 155 | 110.8188 | 150.9199 | −7.54E−01 | −2.71E−01 | 1 | Unchanged Low | |
| | | kinase; 70 k | | | | | | | | | |
| Q15185 | F18cd6 | unactive progesterone | 169.4269 | 72.3 | 110.8534 | 117.54 | −8.12E−01 | −1.23E+00 | 1 | Unchanged Low | |
| O60516 | J18cd3 | eukaryotic transtation | 104.3739 | 147 | 110.9085 | 120.8526 | 8.76E−02 | 4.97E−01 | 1 | Unchanged Low | |
| | | initiation fa | | | | | | | | | |
| Q16651 | J01ab5 | protease: serine; | 334.0495 | 125 | 110.9253 | 190.1399 | −1.59E+00 | −1.41E+00 | 1 | Unchanged Low | |
| | | 8 (prostasin) | | | | | | | | | |
| P48788 | E22cd2 | troponin I; sketetal; | 121.3602 | 110 | 110.9357 | 114.1376 | −1.30E−01 | −1.40E−01 | 1 | Unchanged Low | |
| | | fast | | | | | | | | | |
| Q01968 | N06ab7 | oculocerebrorenal | 169.0079 | 95.9 | 110.9507 | 125.3025 | −6.07E−01 | −8.17E−01 | 1 | Unchanged Low | |
| | | syndrome of L | | | | | | | | | |
| Q9H6Z6 | L11gh7 | hypothetical protein | 115.1546 | 126 | 111.0337 | 117.2667 | −5.26E−02 | 1.25E−01 | 1 | Unchanged Low | |
| | | FLJ21628 | | | | | | | | | |
| Q9NQ55 | B01gh4 | peter pan homolog | 122.449 | 120 | 111.0627 | 117.876 | −1.41E−01 | −3.50E−02 | 1 | Unchanged Low | |
| | | (Drosophila) | | | | | | | | | |
| O95359 | A24gh1 | transforming: acidic | 108.2194 | 139 | 111.0999 | 119.4624 | 3.79E−02 | 3.62E−01 | 1 | Unchanged Low | |
| | | coiled-coil c | | | | | | | | | |
| P01189 | I15ab8 | proopiomelanocortin | 145.0634 | 90.4 | 111.1389 | 116.5442 | −3.84E−01 | −6.82E−01 | 1 | Unchanged Low | |
| | | (adrenocort | | | | | | | | | |
| Q9Y525 | G13cd8 | v-maf musculo- | 119.023 | 141 | 111.1934 | 123.6546 | −9.82E−02 | 2.42E−01 | 1 | Unchanged Low | |
| | | aponeurotic fibros | | | | | | | | | |
| Q9UHC9 | L09cd8 | NPC1 (Niemann-Pick | 107.7403 | 138 | 111.3741 | 118.9021 | 4.79E−02 | 3.53E−01 | 1 | Unchanged Low | |
| | | disease; ty | | | | | | | | | |
| Q9HBT2 | P14gh7 | hypothetical protein | 147.5097 | 98 | 111.4385 | 118.9743 | −4.05E−01 | −5.90E−01 | 1 | Unchanged Low | |
| | | PP1057 | | | | | | | | | |
| O00219 | M22ef1 | hyaluronan synthase 3 | 224.5087 | 89 | 111.4539 | 141.641 | −1.01E+00 | −1.34E+00 | 1 | Unchanged Low | |
| P22735 | M16cd1 | transglutaminase 1 | 136.8808 | 91 | 111.5589 | 113.1407 | −2.95E−01 | −5.89E−01 | 1 | Unchanged Low | |
| | | (K polypeptid | | | | | | | | | |
| O15388 | A11ab4 | sialic acid binding | 105.2847 | 147 | 111.6016 | 121.3043 | 8.41E−02 | 4.82E−01 | 1 | Unchanged Low | |
| | | tg-like lectin 6 | | | | | | | | | |
| Q95389 | J01cd4 | WNT1 inducible | 134.8825 | 94 | 111.6123 | 113.4959 | −2.73E−01 | −5.21E−01 | 1 | Unchanged Low | |
| | | signaling pathw | | | | | | | | | |
| Q06190 | D21ab8 | protein phosphatase | 134.6973 | 102 | 111.8192 | 116.2494 | −2.69E−01 | −3.98E−01 | 1 | Unchanged Low | |
| | | 2 (formerly: | | | | | | | | | |
| P50226 | C03cd3 | sulfotransferase | 147.6874 | 101 | 111.8328 | 120.0792 | −4.01E−01 | −5.52E−01 | 1 | Unchanged Low | |
| | | family; cytosolic | | | | | | | | | |
| Q16632 | N10ab5 | general transcription | 120.7982 | 116 | 111.85 | 116.1383 | −1.00E−01 | −6.14E−02 | 1 | Unchanged Low | |
| | | factor IIIC: | | | | | | | | | |
| P54762 | A24ef7 | EphB1 | 107.3397 | 168 | 111.8669 | 129.1191 | 5.96E−02 | 6.48E−01 | 1 | Unchanged Low | |
| Q15269 | E15cd1 | PWP2 periodic | 125.0688 | 107 | 111.8713 | 114.654 | −1.61E−01 | −2.25E−01 | 1 | Unchanged Low | |
| | | tryptophan protei | | | | | | | | | |
| O14495 | H10cd3 | phosphatidic acid | 120.761 | 109 | 111.9087 | 113.9808 | −1.10E−01 | −1.44E−01 | 1 | Unchanged Low | |
| | | phosphatase t | | | | | | | | | |
| Q9P0U0 | N24ef3 | PC326 protein | 152.5639 | 101 | 111.9543 | 121.9267 | −4.47E−01 | −5.91E−01 | 1 | Unchanged Low | |
| P50120 | J02ab8 | retinol binding | 231.806 | 217 | 111.9639 | 187.0028 | −1.05E+00 | −9.36E−02 | 1 | Unchanged Low | |
| | | protein 2; cellular | | | | | | | | | |
| Q9UHR0 | D08gh1 | G protein-coupled | 111.83 | 151 | 111.9778 | 124.9147 | 1.91E−03 | 4.33E−01 | 1 | Unchanged Low | |
| | | receptor kinas | | | | | | | | | |
| Q13977 | A12ef1 | cerebellar | 105.2052 | 180 | 112.0477 | 132.4319 | 9.09E−02 | 7.75E−01 | 1 | Unchanged Low | |
| | | degeneration-related p | | | | | | | | | |
| P51692 | C18cd8 | signal transducer | 126.7779 | 100 | 112.0645 | 112.9412 | −1.78E−01 | −3.43E−01 | 1 | Unchanged Low | |
| | | and activator o | | | | | | | | | |
| P51164 | N12ab2 | ATPase; H+/K+ | 136.4695 | 112 | 112.2007 | 120.3347 | −2.82E−01 | −2.81E−01 | 1 | Unchanged Low | |
| | | exchanging: beta | | | | | | | | | |
| Q9NXH3 | C02gh2 | protein phosphatase | 113.3081 | 127 | 112.2521 | 117.6836 | −1.35E−02 | 1.70E−01 | 1 | Unchanged Low | |
| | | 1; regulator | | | | | | | | | |
| | C04gh7 | ER to nucleus | 112.9203 | 125 | 112.2701 | 116.8658 | −8.33E−03 | 1.51E−01 | 1 | Unchanged Low | |
| | | signalling 2 | | | | | | | | | |
| P16435 | G16ef6 | P450 (cytochrome) | 110.2003 | 132 | 112.2899 | 118.227 | 2.71E−02 | 2.62E−01 | 1 | Unchanged Low | |
| | | oxidoreducta | | | | | | | | | |
| | E05gh6 | hypothetical protein | 116.1511 | 137 | 112.3522 | 121.7814 | −4.80E−02 | 2.36E−01 | 1 | Unchanged Low | |
| | | MGC5338 | | | | | | | | | |
| Q02080 | L03ab7 | MADS box | 138.6124 | 100 | 112.3745 | 117.0502 | −3.03E−01 | −4.69E−01 | 1 | Unchanged Low | |
| | | transcription enhance | | | | | | | | | |
| Q15334 | J06ab7 | lethal giant | 123.3076 | 110 | 112.3893 | 115.3049 | −1.34E−01 | −1.62E−01 | 1 | Unchanged Low | |
| | | larvae homolog 1 (Dr | | | | | | | | | |
| P10636 | J13ab7 | microtubule-associated | 115.8818 | 142 | 112.4092 | 123.46 | −4.39E−02 | 2.94E−01 | 1 | Unchanged Low | |
| | | protein ta | | | | | | | | | |
| Q9Y4J6 | A01gh2 | zinc finger protein | 138.8767 | 102 | 112.4376 | 117.8247 | −3.05E−01 | −4.43E−01 | 1 | Unchanged Low | |
| | | 6 (CMPX1) | | | | | | | | | |
| Q9UHY7 | P18gh4 | E-1 enzyme | 108.9217 | 134 | 112.5485 | 118.5336 | 4.73E−02 | 3.00E−01 | 1 | Unchanged Low | |
| Q14147 | K04gh1 | DEAD/H (Asp-Glu-Ala- | 117.5358 | 119 | 112.7866 | 116.4348 | −5.95E−02 | 1.76E−02 | 1 | Unchanged Low | |
| | | Asp/His) t | | | | | | | | | |
| O95880 | N06gh4 | KIAA1513 protein | 168.2097 | 104 | 112.9115 | 128.5046 | −5.75E−01 | −6.88E−01 | 1 | Unchanged Low | |
| Q15013 | J23ef3 | gene predicted | 104.556 | 198 | 113.0807 | 138.5918 | 1.13E−01 | 9.22E−01 | 1 | Unchanged Low | |
| | | from cDNA with a | | | | | | | | | |
| Q9P1E2 | E01gh4 | hypothetical protein | 138.6039 | 107 | 113.1066 | 119.6869 | −2.93E−01 | −3.69E−01 | 1 | Unchanged Low | |
| | | PRO2219 | | | | | | | | | |
| P07203 | H23ef1 | glutathione peroxidase | 120.5127 | 114 | 113.1788 | 115.8519 | −9.06E−02 | −8.19E−02 | 1 | Unchanged Low | |
| | | 1 | | | | | | | | | |
| Q13206 | M07ab5 | DEAD/H (Asp-Glu- | 140.5274 | 92.5 | 113.1804 | 115.4153 | −3.12E−01 | −6.03E−01 | 1 | Unchanged Low | |
| | | Ala-Asp/His) t | | | | | | | | | |
| O15085 | L15ef3 | Rho guanine | 121.8405 | 111 | 113.2492 | 115.3055 | −1.05E−01 | −1.37E−01 | 1 | Unchanged Low | |
| | | nucleotide exchang | | | | | | | | | |
| P48426 | C07cd1 | phosphatidylinositol- | 232.9191 | 163 | 113.2533 | 169.6778 | −1.04E+00 | −5.16E−01 | 1 | Unchanged Low | |
| | | 4-phosphate | | | | | | | | | |
| Q9NRQ2 | K20gh4 | phospholipid | 126.2397 | 116 | 113.2849 | 118.4782 | −1.56E−01 | −1.23E−01 | 1 | Unchanged Low | |
| | | scramblase 4 | | | | | | | | | |
| O43171 | B24cd3 | CDC14 cell division | 126.784 | 113 | 113.382 | 117.8862 | −1.61E−01 | −1.60E−01 | 1 | Unchanged Low | |
| | | cycle 14 hon | | | | | | | | | |
| Q9HB90 | M02gh5 | Rag C protein | 132.9037 | 136 | 113.4299 | 127.5658 | −2.29E−01 | 3.71E−02 | 1 | Unchanged Low | |
| Q9H733 | I21gh5 | single Ig IL-1R- | 113.5449 | 137 | 113.4753 | 121.334 | −8.84E−04 | 2.71E−01 | 1 | Unchanged Low | |
| | | related molecule | | | | | | | | | |
| P43353 | C06ab2 | aldehyde | 141.7651 | 101 | 113.5244 | 118.734 | −3.21E−01 | −4.90E−01 | 1 | Unchanged Low | |
| | | dehydrogenase 3 famil | | | | | | | | | |
| Q15053 | G14gh1 | KIAA0040 gene | 111.1705 | 146 | 113.5775 | 123.496 | 3.09E−02 | 3.91E−01 | 1 | Unchanged Low | |
| | | product | | | | | | | | | |
| P22301 | H01ef7 | interleukin 10 | 174.8595 | 108 | 113.5877 | 132.021 | −6.22E−01 | −7.00E−01 | 1 | Unchanged Low | |
| Q9BW47 | N12gh5 | chromosome 20 open | 125.8289 | 154 | 113.6231 | 131.1841 | −1.47E−01 | 2.92E−01 | 1 | Unchanged Low | |
| | | reading fra | | | | | | | | | |
| P21917 | M03ab6 | dopamine receptor D4 | 158.6974 | 151 | 113.7899 | 141.2355 | −4.80E−01 | −6.96E−02 | 1 | Unchanged Low | |
| Q9Y6K5 | M19cd1 | 2′-5′-oligoadenylate | 127.2648 | 142 | 113.9993 | 127.6931 | −1.59E−01 | 1.56E−01 | 1 | Unchanged Low | |
| | | synthetase 3 | | | | | | | | | |
| Q9Y2A4 | N22cd5 | Kruppel-type zinc | 136.0849 | 133 | 114.1396 | 127.5854 | −2.54E−01 | −3.82E−02 | 1 | Unchanged Low | |
| | | finger (C2H2) | | | | | | | | | |
| Q16880 | A16cd3 | UDP glycosyl- | 112.9309 | 133 | 114.1612 | 119.9407 | 1.56E−02 | 2.33E−01 | 1 | Unchanged Low | |
| | | transferase 8 (UDP | | | | | | | | | |
| O95747 | D09cd5 | oxidative-stress | 113.39 | 123 | 114.2384 | 116.7318 | 1.08E−02 | 1.12E−01 | 1 | Unchanged Low | |
| | | responsive 1 | | | | | | | | | |
| P25106 | L12ef4 | G protein-coupled | 100.7123 | 147 | 114.2482 | 120.5368 | 1.82E−01 | 5.42E−01 | 1 | Unchanged Low | |
| | | receptor | | | | | | | | | |
| | H23ab6 | immunoglobulin | 102.6971 | 145 | 114.3219 | 120.6924 | 1.55E−01 | 4.98E−01 | 1 | Unchanged Low | |
| | | superfamily; me | | | | | | | | | |
| P45984 | L18ef5 | mitogen-activated | 139.4168 | 88 | 114.3405 | 113.914 | −2.86E−01 | −6.64E−01 | 1 | Unchanged Low | |
| | | protein kinase | | | | | | | | | |
| Q9NPF7 | I03ef3 | interleukin 23; | 98.83593 | 182 | 114.4153 | 131.5891 | 2.11E−01 | 8.77E−01 | 1 | Unchanged Low | |
| | | alpha subunit p19 | | | | | | | | | |
| Q9UH94 | L07cd8 | prolactin regulatory | 140.4102 | 106 | 114.4158 | 120.3231 | −2.95E−01 | −4.04E−01 | 1 | Unchanged Low | |
| | | element bind | | | | | | | | | |
| Q16559 | F05cd2 | T-cell acute | 131.4974 | 111 | 114.5482 | 118.9991 | −1.99E−01 | −2.45E−01 | 1 | Unchanged Low | |
| | | lymphocytic leukemia | | | | | | | | | |
| Q9UGH0 | C02cd8 | solute carrier family | 98.73308 | 158 | 114.6616 | 123.6748 | 2.16E−01 | 6.75E−01 | 1 | Unchanged Low | |
| | | 17 (anion/su | | | | | | | | | |
| Q15544 | J13cd2 | TAF11 RNA polymerase | 117.4634 | 123 | 114.6792 | 118.4317 | −3.46E−02 | 6.82E−02 | 1 | Unchanged Low | |
| | | II; TATA | | | | | | | | | |
| O00628 | N24ab7 | peroxisomal | 156.16 | 96.9 | 114.7281 | 122.588 | −4.45E−01 | −6.89E−01 | 1 | Unchanged Low | |
| | | biogenesis factor 7 | | | | | | | | | |
| Q43241 | F07ab5 | golgi autoantigen; | 123.9421 | 104 | 114.8381 | 114.422 | −1.10E−01 | −2.46E−01 | 1 | Unchanged Low | |
| | | golgin subfam | | | | | | | | | |
| Q9Y6N1 | M14ab4 | COX11 homolog; | 127.1203 | 100 | 114.8993 | 114.0678 | −1.46E−01 | −3.44E−01 | 1 | Unchanged Low | |
| | | cytochrome c c | | | | | | | | | |
| Q9UHX5 | O01ef4 | HT002 protein; | 98.26523 | 155 | 114.9242 | 122.8348 | 2.26E−01 | 6.60E−01 | 1 | Unchanged Low | |
| | | hypertension-rela | | | | | | | | | |
| Q01664 | A02cd2 | transcription factor | 268.3453 | 66.7 | 115.0377 | 150.0321 | −1.22E+00 | −2.01E+00 | 1 | Unchanged Low | |
| | | AP-4 (activati | | | | | | | | | |
| Q9BUI1 | O03gh6 | leukocyte receptor | 112.2417 | 125 | 115.039 | 117.3611 | 3.55E−02 | 1.53E−01 | 1 | Unchanged Low | |
| | | cluster (LRC) | | | | | | | | | |
| Q9HBL5 | M17gh4 | AD023 protein | 103.9999 | 137 | 115.0443 | 118.5457 | 1.46E−01 | 3.93E−01 | 1 | Unchanged Low | |
| Q9NXV2 | N08gh2 | hypothetical protein | 126.6647 | 147 | 115.1386 | 129.6462 | −1.38E−01 | 2.16E−01 | 1 | Unchanged Low | |
| | | FLJ20040 | | | | | | | | | |
| Q15424 | D08cd1 | scaffold attachment | 134.9613 | 92.7 | 115.1845 | 114.2781 | −2.29E−01 | −5.42E−01 | 1 | Unchanged Low | |
| | | factor B | | | | | | | | | |
| P26440 | FO6ab6 | isovaleryl Coenzyme | 178.7093 | 115 | 115.2412 | 136.2534 | −6.33E−01 | −6.38E−01 | 1 | Unchanged Low | |
| | | A dehydrog | | | | | | | | | |
| O60264 | J11cd3 | SWI/SNF related; | 105.7775 | 132 | 115.2498 | 117.7402 | 1.24E−01 | 3.22E−01 | 1 | Unchanged Low | |
| | | metrix associa | | | | | | | | | |
| Q15286 | K07cd7 | RAB35; member RAS | 138.2766 | 183 | 115.3443 | 145.6 | −2.62E−01 | 4.06E−01 | 1 | Unchanged Low | |
| | | oncogene | | | | | | | | | |
| Q9UGK0 | I10ef4 | DIPB protein | 303.2745 | 172 | 115.4096 | 196.7868 | −1.39E+00 | −8.21E−01 | 1 | Unchanged Low | |
| Q92980 | J06cd5 | putative protein | 142.089 | 181 | 115.4166 | 146.2003 | −3.00E−01 | 3.50E−01 | 1 | Unchanged Low | |
| | | similar to nessy | | | | | | | | | |
| Q99726 | M06cd3 | solute carrier family | 112.6008 | 121 | 115.4854 | 116.3247 | 3.39E−02 | 9.75E−02 | 1 | Unchanged Low | |
| | | 30 (zinc tran | | | | | | | | | |
| P01111 | O17ab8 | neuroblastoma RAS | 123.6283 | 108 | 115.5932 | 115.8629 | −9.70E−02 | −1.90E−01 | 1 | Unchanged Low | |
| | | viral (v-ras) | | | | | | | | | |
| Q92574 | G13cd3 | tuberous sclerosis 1 | 109.8519 | 128 | 115.7107 | 117.8303 | 7.50E−02 | 2.20E−01 | 1 | Unchanged Low | |
| P16106 | C01gh7 | H3 histone family; | 134.9364 | 118 | 115.868 | 122.8016 | −2.20E−01 | −1.98E−01 | 1 | Unchanged Low | |
| | | member I | | | | | | | | | |
| Q9NWX5 | O24gh2 | hypothetical protein | 118.1581 | 112 | 115.9919 | 115.5043 | −2.67E−02 | −7.26E−02 | 1 | Unchanged Low | |
| | | FLJ20548 | | | | | | | | | |
| Q9Y6B7 | M05ab3 | adaptor-retated | 217.8572 | 170 | 116.0173 | 168.0108 | −9.09E−01 | −3.57E−01 | 1 | Unchanged Low | |
| | | protein complex | | | | | | | | | |
| Q9Y4F1 | J04cd5 | FERM; RhoGEF | 108.6215 | 130 | 116.0342 | 118.0819 | 9.52E−02 | 2.55E−01 | 1 | Unchanged Low | |
| | | (ARHGEF) and | | | | | | | | | |
| Q06710 | M14cd3 | paired box gene 8 | 120.1357 | 129 | 116.1006 | 121.7641 | −4.93E−02 | 1.03E−01 | 1 | Unchanged Low | |
| Q9H6N1 | J17gh5 | hypothetical protein | 214.4909 | 242 | 116.2509 | 190.8582 | −8.84E−01 | 1.73E−01 | 1 | Unchanged Low | |
| | | FLJ22056 | | | | | | | | | |
| Q9NSY1 | K09gh3 | homolog of mouse | 111.9034 | 123 | 116.2514 | 117.0914 | 5.50E−02 | 1.38E−01 | 1 | Unchanged Low | |
| | | BMP-2 inducil | | | | | | | | | |
| Q14106 | H20cd6 | transducer of ERBB2; | 117.9634 | 134 | 116.2536 | 122.8358 | −2.11E−02 | 1.67E−01 | 1 | Unchanged Low | |
| | | 2 | | | | | | | | | |
| Q9UK11 | B02ef4 | zinc finger protein | 119.1221 | 133 | 116.2873 | 122.8322 | −3.47E−02 | 1.60E−01 | 1 | Unchanged Low | |
| | | 223 | | | | | | | | | |
| P08069 | O17ef6 | insulin-like growth | 106.3434 | 178 | 116.3103 | 133.4883 | 1.29E−01 | 7.42E−01 | 1 | Unchanged Low | |
| | | factor 1 recep | | | | | | | | | |
| Q9H6F5 | I21gh6 | hypothetical protein | 120.5097 | 112 | 116.3129 | 116.1638 | −5.11E−02 | −1.10E−01 | 1 | Unchanged Low | |
| | | MGC2574 | | | | | | | | | |
| 014373 | O04ab3 | galactose-1-phosphate | 157.7476 | 231 | 116.3555 | 168.3955 | −4.39E−01 | 5.51E−01 | 1 | Unchanged Low | |
| | | uridylyltra | | | | | | | | | |
| P20393 | G04ef5 | nuclear receptor | 107.1138 | 138 | 116.4228 | 120.571 | 1.20E−01 | 3.67E−01 | 1 | Unchanged Low | |
| | | subfamily 1: gro | | | | | | | | | |
| P36915 | O01ab5 | guanine nucleotide | 120.9306 | 111 | 116.4819 | 116.2706 | −5.41E−02 | −1.18E−01 | 1 | Unchanged Low | |
| | | binding prote | | | | | | | | | |
| O95109 | G02cd8 | chromosome 20 open | 143.5728 | 103 | 116.5321 | 121.0452 | −3.01E−01 | −4.79E−01 | 1 | Unchanged Low | |
| | | reading fra | | | | | | | | | |
| Q9NVS2 | I24gh3 | mitochondrial | 117.9573 | 124 | 116.6011 | 119.5497 | 1.67E−02 | 7.31E−02 | 1 | Unchanged Low | |
| | | ribosomal protein | | | | | | | | | |
| Q92950 | B08cd6 | CUG triplet repeat; | 109.8876 | 129 | 116.7366 | 118.6125 | 8.72E−02 | 2.34E−01 | 1 | Unchanged Low | |
| | | RNA binding | | | | | | | | | |
| Q14977 | C04ab7 | SP110 nuclear body | 107.0969 | 158 | 116.8973 | 127.3783 | 1.26E−01 | 5.62E−01 | 1 | Unchanged Low | |
| | | protein | | | | | | | | | |
| P16106 | O04gh6 | H3 histone family; | 94.76745 | 159 | 116.9796 | 123.5365 | 3.04E−01 | 7.45E−01 | 1 | Unchanged Low | |
| | | member A | | | | | | | | | |
| Q9Y3C1 | N18ef2 | hypothetical protein | 145.1935 | 164 | 117.0185 | 141.934 | −3.11E−01 | 1.72E−01 | 1 | Unchanged Low | |
| | | HSPC111 | | | | | | | | | |
| P37287 | C11ef1 | phosphatidylinositol | 132.5354 | 109 | 117.2467 | 119.4716 | −1.77E−01 | −2.87E−01 | 1 | Unchanged Low | |
| | | glycan; class | | | | | | | | | |
| Q9UIG5 | G05ef4 | SEEK1 protein | 102.5149 | 148 | 117.279 | 122.443 | 1.94E−01 | 5.25E−01 | 1 | Unchanged Low | |
| Q9UNE2 | G15cd6 | rabphilin 3A-like | 110.3023 | 123 | 117.2874 | 117.0111 | 8.86E−02 | 1.62E−01 | 1 | Unchanged Low | |
| | | (without C2 dom | | | | | | | | | |
| Q14123 | A17cd1 | phosphodiesterase | 121.3078 | 132 | 117.3754 | 123.5304 | −4.75E−02 | 1.21E−01 | 1 | Unchanged Low | |
| | | 1C; calmodul | | | | | | | | | |
| Q16206 | P03ab2 | cytosolic ovarian | 116.5183 | 157 | 117.4492 | 130.1671 | 1.15E−02 | 4.26E−01 | 1 | Unchanged Low | |
| | | carcinoma antig | | | | | | | | | |
| Q9NWT1 | D21gh2 | hypothetical protein | 116.0517 | 116 | 117.4816 | 116.4708 | 1.77E−02 | −2.15E−03 | 1 | Unchanged Low | |
| | | FLJ20624 | | | | | | | | | |
| Q12756 | C05ab3 | axonal transport | 138.4398 | 232 | 117.5489 | 162.7767 | −2.36E−01 | 7.47E−01 | 1 | Unchanged Low | |
| | | of synaptic vesi | | | | | | | | | |
| P28288 | B10ab8 | ATP-binding cassette; | 140.4464 | 95.9 | 117.5859 | 117.9674 | −2.56E−01 | −5.S1E−01 | 1 | Unchanged Low | |
| | | sub-family | | | | | | | | | |
| P05198 | G08ab5 | eukaryotic translation | 115.5074 | 119 | 117.6033 | 117.2286 | 2.59E−02 | 3.77E−02 | 1 | Unchanged Low | |
| | | initiation fa | | | | | | | | | |
| Q9H0V9 | D08gh7 | hypothetical protein | 128.832 | 101 | 117.6689 | 115.9248 | −1.31E−01 | −3.47E−01 | 1 | Unchanged Low | |
| | | DKFZp564L: | | | | | | | | | |
| Q92984 | M10ef1 | interferon-induced | 258.0737 | 221 | 117.8182 | 198.9799 | −1.13E+00 | −2.23E−01 | 1 | Unchanged Low | |
| | | protein 35 | | | | | | | | | |
| O95498 | A13cd5 | vanin 2 | 109.7731 | 123 | 117.8273 | 116.8572 | 1.02E−01 | 1.64E−01 | 1 | Unchanged Low | |
| Q9H1E1 | J01gh8 | ribonuclease 7 | 109.332 | 131 | 117.8377 | 119.4295 | 1.08E−01 | 2.62E−01 | 1 | Unchanged Low | |
| Q9NV88 | H01gh3 | hypothetical protein | 108.5849 | 125 | 117.8411 | 117.172 | 1.18E−01 | 2.04E−01 | 1 | Unchanged Low | |
| | | FLJ10871 | | | | | | | | | |
| Q9Y310 | F06cd6 | SnRNP assembly | 119.9305 | 113 | 117.8563 | 117.0556 | −2.52E−02 | −8.10E−02 | 1 | Unchanged Low | |
| | | defective 1 ho | | | | | | | | | |
| P48382 | O06cd1 | regulatory factor | 191.5142 | 122 | 118.0773 | 143.9771 | −6.98E−01 | −6.47E−01 | 1 | Unchanged Low | |
| | | X; 5 (influences | | | | | | | | | |
| P52738 | K04cd3 | zinc finger protein | 136.6586 | 154 | 118.1501 | 136.2205 | −2.10E−01 | 1.71E−01 | 1 | Unchanged Low | |
| | | 140 (clone pH | | | | | | | | | |
| Q9NRW1 | G23ef3 | RAB6B; member RAS | 100.578 | 179 | 118.1659 | 132.4668 | 2.32E−01 | 8.29E−01 | 1 | Unchanged Low | |
| | | oncogene | | | | | | | | | |
| O15298 | A02ab3 | basic leucine zipper | 266.9887 | 110 | 118.2062 | 165.1797 | −1.18E+00 | −1.27E+00 | 1 | Unchanged Low | |
| | | nuclear fact | | | | | | | | | |
| Q9NUN5 | D14gh3 | hypothetical protein | 114.5392 | 116 | 116.2691 | 116.1573 | 4.62E−02 | 1.41E−02 | 1 | Unchanged Low | |
| | | FLJ11240 | | | | | | | | | |
| Q07699 | L03cd1 | sodium channel: | 116.5431 | 113 | 118.3239 | 115.8757 | 2.19E−02 | −4.76E−02 | 1 | Unchanged Low | |
| | | voltage-gated: t | | | | | | | | | |
| P27701 | F10ab6 | kangai 1 (suppression | 116.2761 | 106 | 118.3881 | 114.2624 | 2.60E−02 | −1.05E−01 | 1 | Unchanged Low | |
| | | of tumorig | | | | | | | | | |
| Q9UER5 | O02ef3 | TNF-induced protein | 104.9755 | 131 | 118.4653 | 118.2694 | 1.74E−01 | 3.24E−01 | 1 | Unchanged Low | |
| Q8TAC6 | I24gh7 | chromosome 11 open | 117.4257 | 138 | 118.5887 | 124.5089 | 1.42E−02 | 2.28E−01 | 1 | Unchanged Low | |
| | | reading fra | | | | | | | | | |
| Q9NRW9 | K16gh4 | angiotensin II; type | 136.6357 | 105 | 118.6777 | 120.1901 | −2.03E−01 | −3.76E−01 | 1 | Unchanged Low | |
| | | I receptor-as | | | | | | | | | |
| O95147 | O15cd7 | dual specificity | 181.9968 | 200 | 118.7007 | 166.9415 | −6.17E−01 | 1.37E−01 | 1 | Unchanged Low | |
| | | phosphatase 14 | | | | | | | | | |
| Q13895 | A19ef7 | bystin-like | 108.0232 | 124 | 118.7967 | 116.9004 | 1.37E−01 | 1.98E−01 | 1 | Unchanged Low | |
| P31645 | N03ef5 | solute carrier family | 114.6464 | 189 | 118.8467 | 140.7231 | 5.19E−02 | 7.19E−01 | 1 | Unchanged Low | |
| | | 6 (neurotran | | | | | | | | | |
| P51511 | N17ef7 | matrix metallo- | 122.9018 | 125 | 118.8609 | 122.1739 | −4.82E−02 | 2.16E−02 | 1 | Unchanged Low | |
| | | proteinase 15 (mer | | | | | | | | | |
| Q15154 | O11cd1 | pericentriolar | 127.8378 | 139 | 118.8759 | 128.7076 | −1.05E−01 | 1.25E−01 | 1 | Unchanged Low | |
| | | material 1 | | | | | | | | | |
| Q00007 | E17ef6 | protein phosphatase | 108.364 | 123 | 118.9838 | 116.7703 | 1.35E−01 | 1.82E−01 | 1 | Unchanged Low | |
| | | 2 (formerly: | | | | | | | | | |
| P55822 | L08cd2 | SH3 domain binding | 131.7189 | 133 | 119.0263 | 128.0275 | −1.46E−01 | 1.76E−02 | 1 | Unchanged Low | |
| | | glutamic aci | | | | | | | | | |
| Q14442 | L20ab8 | phosphatidylinositol | 121.0635 | 114 | 119.0306 | 118.0476 | −2.44E−02 | −8.61E−02 | 1 | Unchanged Low | |
| | | glycan; class | | | | | | | | | |
| P54792 | L04ab4 | dishevelled; dsh | 126.677 | 113 | 119.067 | 119.4338 | −8.94E−02 | −1.70E−01 | 1 | Unchanged Low | |
| | | homolog 1 (Dro | | | | | | | | | |
| P42336 | A03ef6 | phosphoinositide-3- | 101.0905 | 143 | 119.1581 | 121.01 | 2.37E−01 | 4.98E−01 | 1 | Unchanged Low | |
| | | kinase; catal | | | | | | | | | |
| Q9UMQ3 | G15ab3 | BarH-like homeobox 2 | 99.5713 | 179 | 119.1741 | 132.6879 | 2.59E−01 | 8.49E−01 | 1 | Unchanged Low | |
| Q9UPY5 | M04ef3 | solute carrier | 173.9407 | 69.5 | 119.3079 | 120.9226 | −5.44E−01 | −1.32E+00 | 1 | Unchanged Low | |
| | | family 7; (cationic a | | | | | | | | | |
| P19113 | H01ef6 | histidine | 111.9277 | 119 | 119.3155 | 116.6743 | 9.22E−02 | 8.57E−02 | 1 | Unchanged Low | |
| | | decarboxylase | | | | | | | | | |
| O95394 | B16ef3 | N-acetylgluco- | 124.8164 | 112 | 119.3184 | 118.8198 | −6.50E−02 | −1.52E−01 | 1 | Unchanged Low | |
| | | samine-phosphate | | | | | | | | | |
| P22557 | A14ab2 | aminolevulinate; | 202.1179 | 129 | 119.3857 | 150.2281 | −7.60E−01 | −6.46E−01 | 1 | Unchanged Low | |
| | | delta-; synthase | | | | | | | | | |
| Q9Y3A2 | G10ef2 | CGI-94 protein | 108.7497 | 167 | 119.4751 | 131.6512 | 1.36E−01 | 6.16E−01 | 1 | Unchanged Low | |
| Q9H5J4 | I05gh6 | long-chain fatty- | 135.0119 | 87 | 119.5181 | 113.8461 | −1.76E−01 | −6.34E−01 | 1 | Unchanged Low | |
| | | acyl elongase | | | | | | | | | |
| P16083 | E15ab8 | NAD(P)H dehydro- | 154.7986 | 95.6 | 119.5724 | 124.3296 | −3.73E−01 | −6.50E−01 | 1 | Unchanged Low | |
| | | genase; guino | | | | | | | | | |
| Q99819 | D02ab2 | Rho GDP dissociation | 125.7962 | 106 | 119.5853 | 117.2457 | −7.30E−02 | −2.42E−01 | 1 | Unchanged Low | |
| | | inhibitor ( | | | | | | | | | |
| O75569 | F06cd3 | protein kinase; | 100.3086 | 137 | 119.6257 | 118.9057 | 2.54E−01 | 4.47E−01 | 1 | Unchanged Low | |
| | | interferon-inducib | | | | | | | | | |
| O15038 | F12ef7 | serine/arginine | 125.19 | 96.6 | 119.719 | 113.8294 | −6.45E−02 | −3.74E−01 | 1 | Unchanged Low | |
| | | repetitive matrix | | | | | | | | | |
| Q9HBH5 | K09gh4 | PAN2 protein | 102.0601 | 139 | 119.7321 | 120.3437 | 2.30E−01 | 4.48E−01 | 1 | Unchanged Low | |
| P41231 | C22ab8 | purinergic receptor | 131.9219 | 130 | 119.6105 | 127.3275 | −1.39E−01 | −1.84E−02 | 1 | Unchanged Low | |
| | | P2Y; G-prote | | | | | | | | | |
| O15397 | D01cd6 | RAN binding protein 8 | 114.6925 | 118 | 119.9197 | 117.4304 | 6.43E−02 | 3.71E−02 | 1 | Unchanged Low | |
| Q9UDY8 | C17cd7 | mucosa associated | 114.4769 | 120 | 119.9403 | 118.129 | 6.73E−02 | 6.76E−02 | 1 | Unchanged Low | |
| | | lymphoid tiss | | | | | | | | | |
| O95819 | E22cd5 | mitogen-activated | 92.42403 | 156 | 119.956 | 122.7769 | 3.76E−01 | 7.55E−01 | 1 | Unchanged Low | |
| | | protein kinase | | | | | | | | | |
| Q12999 | J01ef5 | sarcoma amplified | 114.9397 | 110 | 119.9579 | 114.8641 | 6.17E−02 | −6.74E−02 | 1 | Unchanged Low | |
| | | sequence | | | | | | | | | |
| P25440 | D07cd2 | bromodomain | 116.6707 | 108 | 120.0152 | 114.7701 | 4.08E−02 | −1.16E−01 | 1 | Unchanged Low | |
| | | containing 2 | | | | | | | | | |
| P20827 | H21ef7 | ephrin-A1 | 298.0458 | 760 | 120.3333 | 392.8203 | −1.31E+00 | 1.35E+00 | 1 | Unchanged Low | |
| Q9H652 | P20gh8 | hypothetical protein | 129.5733 | 107 | 120.4428 | 118.9065 | −1.05E−01 | −2.80E−01 | 1 | Unchanged Low | |
| | | MGC4171 | | | | | | | | | |
| P31483 | L20ef4 | TIA1 cytotoxic | 105.4425 | 129 | 120.5386 | 118.2227 | 1.93E−01 | 2.87E−01 | 1 | Unchanged Low | |
| | | granule-associate | | | | | | | | | |
| Q98SM8 | G15gh8 | hypothetical protein | 148.2279 | 273 | 120.573 | 180.6391 | −2.98E−01 | 8.82E−01 | 1 | Unchanged Low | |
| | | MGC10812 | | | | | | | | | |
| P36897 | F04ef5 | transforming growth | 115.7567 | 108 | 120.6724 | 114.8546 | 6.00E−02 | −9.83E−02 | 1 | Unchanged Low | |
| | | factor: beta | | | | | | | | | |
| Q9UMR9 | E08ef3 | YME1-like 1 | 256.0314 | 197 | 120.7227 | 191.0958 | −1.08E+00 | −3.82E−01 | 1 | Unchanged Low | |
| | | (S. cerevisiae) | | | | | | | | | |
| Q92560 | G11ab3 | BRCA1 associated | 101.36 | 161 | 120.7575 | 127.8687 | 2.53E−01 | 6.72E−01 | 1 | Unchanged Low | |
| | | protein-1 (ubi | | | | | | | | | |
| Q9NWU1 | D05gh2 | hypothetical protein | 99.76081 | 154 | 120.7609 | 124.8325 | 2.76E−01 | 6.26E−01 | 1 | Unchanged Low | |
| | | FLJ20604 | | | | | | | | | |
| Q92696 | N14ab8 | Rab geranylgeranyl- | 222.7157 | 272 | 120.7706 | 205.3234 | −8.83E−01 | 2.91E−01 | 1 | Unchanged Low | |
| | | transferase; | | | | | | | | | |
| O95236 | M24ef3 | apolipoprotein L; 3 | 106.592 | 130 | 120.798 | 119.1528 | 1.80E−01 | 2.87E−01 | 1 | Unchanged Low | |
| Q13202 | E21ab5 | dual specificity | 146.3963 | 129 | 120.7987 | 131.9045 | −2.77E−01 | −1.88E−01 | 1 | Unchanged Low | |
| | | phosphatase 8 | | | | | | | | | |
| P11277 | M02cd1 | spectrin; beta; | 112.72 | 114 | 120.8055 | 116.0084 | 9.99E−02 | 2.26E−02 | 1 | Unchanged Low | |
| | | erythrocytic (inclu | | | | | | | | | |
| O95397 | P18ef1 | putative glycolipid | 99.78536 | 143 | 120.8708 | 121.1071 | 2.77E−01 | 5.16E−01 | 1 | Unchanged Low | |
| | | transfer protei | | | | | | | | | |
| P05423 | A20ab3 | BN51 (BHK21) | 131.8122 | 95.5 | 120.9201 | 116.0734 | −1.24E−01 | −4.85E−01 | 1 | Unchanged Low | |
| | | temperature sens | | | | | | | | | |
| Q14338 | I17ab8 | fucosyltransferase 2 | 99.71915 | 181 | 120.9483 | 133.9938 | 2.78E−01 | 8.63E−01 | 1 | Unchanged Low | |
| | | (secretor sta | | | | | | | | | |
| Q9BYI8 | A22gh8 | AIE-75 binding | 90.11911 | 164 | 121.0209 | 125.1864 | 4.25E−01 | 8.67E−01 | 1 | Unchanged Low | |
| | | protein protein | | | | | | | | | |
| Q06203 | D01ab5 | phosphoribosyl | 216.3154 | 105 | 121.029 | 147.3953 | −8.38E−01 | −1.04E+00 | 1 | Unchanged Low | |
| | | pyrophosphate a | | | | | | | | | |
| Q9NZ33 | P10gh4 | brain expressed; | 113.8613 | 162 | 121.0508 | 132.4428 | 8.83E−02 | 5.12E−01 | 1 | Unchanged Low | |
| | | X-linked 1 | | | | | | | | | |
| P04106 | O10ab6 | histidine-rich | 116.1305 | 116 | 121.1163 | 117.5882 | 6.06E−02 | −7.63E−03 | 1 | Unchanged Low | |
| | | glycoprotein | | | | | | | | | |
| O96029 | F03cd7 | opioid growth factor | 201.3579 | 93.5 | 121.2888 | 138.7174 | −7.31E−01 | −1.11E+00 | 1 | Unchanged Low | |
| | | receptor | | | | | | | | | |
| Q01196 | I16ab5 | runt-related | 441.1217 | 136 | 121.3316 | 232.9078 | −1.86E+00 | −1.09E+00 | 1 | Unchanged Low | |
| | | transcription factor 1 | | | | | | | | | |
| Q9UHL4 | J17cd8 | dipeptidylpeptidase 7 | 180.3445 | 191 | 121.351 | 164.207 | −5.72E−01 | 8.23E−02 | 1 | Unchanged Low | |
| P55211 | P16ab3 | caspase 9; apoptosis- | 113.5416 | 111 | 121.3742 | 115.3899 | 9.62E−02 | −2.94E−02 | 1 | Unchanged Low | |
| | | related cys | | | | | | | | | |
| P21283 | A07ab3 | ATPase; H+ | 288.6034 | 288 | 121.3828 | 232.7446 | −1.25E+00 | −1.78E−03 | 1 | Unchanged Low | |
| | | transporting: lysos | | | | | | | | | |
| O43422 | P02ab8 | protein-kinase; | 131.8635 | 100 | 121.304 | 117.7416 | −1.19E−01 | −4.00E−01 | 1 | Unchanged Low | |
| | | interferon-inducib | | | | | | | | | |
| P78406 | L01cd3 | RAE1 RNA export 1 | 147.1192 | 97.1 | 121.4625 | 121.9098 | −2.76E−01 | −5.99E−01 | 1 | Unchanged Low | |
| | | homolog (S. | | | | | | | | | |
| P43362 | J18ab7 | melanoma antigen; | 99.05783 | 139 | 121.5326 | 119.97 | 2.95E−01 | 4.92E−01 | 1 | Unchanged Low | |
| | | family A; 9 | | | | | | | | | |
| Q9H693 | E12gh6 | hypothetical protein | 171.7699 | 96.4 | 121.6097 | 129.9396 | −4.98E−01 | −8.33E−01 | 1 | Unchanged Low | |
| | | FLJ22477 | | | | | | | | | |
| Q9UJ83 | A11cd8 | 2-hydroxyphytanoyl- | 99.45467 | 141 | 121.6399 | 120.5495 | 2.91E−01 | 4.99E−01 | 1 | Unchanged Low | |
| | | CoA lyase | | | | | | | | | |
| Q05048 | I15ab4 | cleavage stimulation | 124.1609 | 105 | 121.6764 | 117.088 | −2.92E−02 | −2.36E−01 | 1 | Unchanged Low | |
| | | factor; 3′ pr | | | | | | | | | |
| P19784 | D11ab4 | casein kinase 2; alpha | 94.68573 | 152 | 121.7819 | 122.6579 | 3.63E−01 | 6.78E−01 | 1 | Unchanged Low | |
| | | prime poly | | | | | | | | | |
| Q9BUX8 | D06gh6 | homolog of yeast | 167.4752 | 178 | 121.8214 | 155.6701 | −4.59E−01 | 8.56E−02 | 1 | Unchanged Low | |
| | | ribosome bioge | | | | | | | | | |
| Q9Y5P6 | D23cd8 | GDP-mannose | 186.4572 | 89.4 | 121.8219 | 125.8828 | −4.50E−01 | −8.97E−01 | 1 | Unchanged Low | |
| | | pyrophosphorylas | | | | | | | | | |
| Q9Y3E1 | F16ef1 | hepatoma-derived | 97.00713 | 148 | 121.9163 | 122.172 | 3.30E−01 | 6.05E−01 | 1 | Unchanged Low | |
| | | growth factor: | | | | | | | | | |
| P40426 | O07cd1 | pre-B-cell leukemia | 122.6888 | 94.8 | 121.9253 | 113.1427 | −9.01E−03 | −3.72E−01 | 1 | Unchanged Low | |
| | | transcription | | | | | | | | | |
| P11217 | K01cd1 | phosphorylase; | 314.8041 | 110 | 122.008 | 182.286 | −1.37E+00 | −1.52E+00 | 1 | Unchanged Low | |
| | | glycogen; muscle | | | | | | | | | |
| Q16563 | D18cd2 | synaptophysin- | 97.62823 | 144 | 122.0944 | 121.3173 | 3.23E−01 | 5.63E−01 | 1 | Unchanged Low | |
| | | like protein | | | | | | | | | |
| Q95698 | L07ab6 | inhibitor of growth | 102.2388 | 135 | 122.1358 | 119.6732 | 2.57E−01 | 3.97E−01 | 1 | Unchanged Low | |
| | | family; membe | | | | | | | | | |
| P19447 | K24ef6 | excision repair cross- | 95.41189 | 148 | 122.1425 | 121.7269 | 3.56E−01 | 6.30E−01 | 1 | Unchanged Low | |
| | | complemen | | | | | | | | | |
| Q9UK39 | D08cd7 | CCR4 carbon catabo- | 173.8761 | 262 | 122.1589 | 185.8825 | −5.09E−01 | 5.89E−01 | 1 | Unchanged Low | |
| | | lite repressi | | | | | | | | | |
| Q15785 | E23cd7 | translocase of outer | 296.8521 | 144 | 122.1776 | 187.6861 | −1.28E+00 | −1.04E+00 | 1 | Unchanged Low | |
| | | mitochondria | | | | | | | | | |
| Q99571 | C14ab8 | purinergic receptor | 418.1812 | 410 | 122.3053 | 316.7241 | −1.77E+00 | −2.96E−02 | 1 | Unchanged Low | |
| | | P2X; tigasd-g | | | | | | | | | |
| Q9BTY2 | E24gh8 | hypothetical protein | 131.9449 | 91.5 | 122.3524 | 115.258 | −1.09E−01 | −5.28E−01 | 1 | Unchanged Low | |
| | | MGC1314 si | | | | | | | | | |
| Q9P0P1 | D12ef1 | hypothetical protein | 147.9172 | 191 | 122.4861 | 153.8845 | −2.72E−01 | 3.71E−01 | 1 | Unchanged Low | |
| | | FLJ10769 | | | | | | | | | |
| Q9Y324 | P01ef1 | CGI-35 protein | 129.1118 | 97 | 122.5531 | 116.2093 | −7.52E−02 | −4.13E−01 | 1 | Unchanged Low | |
| P37286 | O17cd3 | ubiquitin-conjugating | 98.88763 | 165 | 122.5669 | 128.7921 | 3.10E−01 | 7.38E−01 | 1 | Unchanged Low | |
| | | enzyme E2 | | | | | | | | | |
| Q00577 | O07ef5 | purine-rich element | 116.0341 | 123 | 122.5705 | 120.658 | 7.91E−02 | 8.84E−02 | 1 | Unchanged Low | |
| | | binding prote | | | | | | | | | |
| O75152 | L21gh1 | KIAA0663 gene | 101.4464 | 140 | 122.5789 | 121.2139 | 2.73E−01 | 4.61E−01 | 1 | Unchanged Low | |
| | | product | | | | | | | | | |
| P23610 | H06cd7 | coagulation factor | 164.911 | 103 | 122.5902 | 130.3149 | −4.28E−01 | −6.73E−01 | 1 | Unchanged Low | |
| | | VIII-associate | | | | | | | | | |
| O75436 | M20cd5 | vacuolar protein | 118.1135 | 108 | 122.6522 | 116.2363 | 5.44E−02 | −1.30E−01 | 1 | Unchanged Low | |
| | | sorting 26 (yeas | | | | | | | | | |
| Q9HA40 | B23gh5 | hypothetical protein | 104.6286 | 126 | 122.666 | 117.8494 | 2.29E−01 | 2.71E−01 | 1 | Unchanged Low | |
| | | FLJ12287 si | | | | | | | | | |
| Q9NX62 | I20gh2 | hypothetical protein | 103.7764 | 136 | 122.701 | 120.7522 | 2.42E−01 | 3.88E−01 | 1 | Unchanged Low | |
| | | FLJ20421 | | | | | | | | | |
| O76062 | E04cd2 | transmembrane 7 | 301.1903 | 215 | 122.7645 | 212.8749 | −1.29E+00 | −4.89E−01 | 1 | Unchanged Low | |
| | | superfamily m | | | | | | | | | |
| Q16445 | H02ab3 | gamma-aminobutyric | 150.641 | 87.5 | 122.7829 | 120.3177 | −2.95E−01 | −7.83E−01 | 1 | Unchanged Low | |
| | | acid (GABA | | | | | | | | | |
| Q08462 | G11ef6 | adenylate cyclase | 107.541 | 146 | 122.8502 | 125.3933 | 1.92E−01 | 4.39E−01 | 1 | Unchanged Low | |
| | | 2 (brain) | | | | | | | | | |
| P78560 | A22ef6 | CASP2 and RIPK1 | 108.4508 | 153 | 122.8627 | 128.1152 | 1.80E−01 | 4.97E−01 | 1 | Unchanged Low | |
| | | domain conta | | | | | | | | | |
| P19634 | N17ef5 | solute carrier | 118.9969 | 99 | 122.898 | 113.6214 | 4.65E−02 | −2.66E−01 | 1 | Unchanged Low | |
| | | family 9(sodium/hy | | | | | | | | | |
| P28370 | N12cd1 | SWI/SNF related; | 115.471 | 100 | 122.9184 | 114.9572 | 9.02E−02 | −1.17E−01 | 1 | Unchanged Low | |
| | | matrix associa | | | | | | | | | |
| Q92503 | H06cd1 | SEC14-like 1 | 151.5939 | 151 | 122.9424 | 141.9225 | −3.02E−01 | −3.46E−03 | 1 | Unchanged Low | |
| | | (S. cerevisiae) | | | | | | | | | |
| Q9UL02 | H24ef1 | L-kynurenine/ | 105.6951 | 124 | 123.0487 | 117.4294 | 2.19E−01 | 2.25E−01 | 1 | Unchanged Low | |
| | | alpha-aminoadipat | | | | | | | | | |
| Q9Y5T5 | J13cd6 | ubiquitin specific | 117.1056 | 120 | 123.0646 | 120.0007 | 7.16E−02 | 3.32E−02 | 1 | Unchanged Low | |
| | | protease 16 | | | | | | | | | |
| | G03gh8 | protocadherin 15 | 153.0978 | 126 | 123.1548 | 134.1729 | −3.14E−01 | −2.78E−01 | 1 | Unchanged Low | |
| P15848 | H02ab2 | arylsulfatase B | 149.4791 | 95.6 | 123.1651 | 122.7499 | −2.79E−01 | −6.45E−01 | 1 | Unchanged Low | |
| Q9UP66 | P24ab6 | low density lipoprotein | 114.2435 | 124 | 123.2083 | 120.4437 | 1.09E−01 | 1.17E−01 | 1 | Unchanged Low | |
| | | receptor-r | | | | | | | | | |
| O75160 | P07gh1 | KIAA0672 gene | 121.9398 | 117 | 123.2554 | 120.8501 | 1.55E−02 | −5.53E−02 | 1 | Unchanged Low | |
| | | product | | | | | | | | | |
| P78385 | L14ab6 | keratin; hair; | 100.703 | 135 | 123.2569 | 119.6793 | 2.92E−01 | 4.24E−01 | 1 | Unchanged Low | |
| | | basic; 3 | | | | | | | | | |
| O94967 | B21ef8 | KIAA0893 protein | 92.15324 | 158 | 123.2707 | 124.6247 | 4.20E−01 | 7.82E−01 | 1 | Unchanged Low | |
| O95719 | K01cd5 | xenotropic and | 108.0877 | 122 | 123.3497 | 117.7043 | 1.91E−01 | 1.71E−01 | 1 | Unchanged Low | |
| | | polytropic retrovi | | | | | | | | | |
| P48147 | F09ab8 | prolyl endopeptidase | 130.0641 | 129 | 123.3564 | 127.6048 | −7.64E−02 | −7.45E−03 | 1 | Unchanged Low | |
| P20231 | B15gh7 | tryptase beta 2 | 106.6865 | 130 | 123.4905 | 119.9323 | 2.11E−01 | 2.81E−01 | 1 | Unchanged Low | |
| 095299 | G02ab7 | NADH dehydrogenase | 107.385 | 129 | 123.5991 | 119.8634 | 2.03E−01 | 2.60E−01 | 1 | Unchanged Low | |
| | | (ubiguino | | | | | | | | | |
| O75033 | I18gh1 | KIAA0445 gene product | 121.5196 | 122 | 123.624 | 122.4835 | 2.48E−02 | 9.32E−03 | 1 | Unchanged Low | |
| P51668 | D03cd4 | ubiquitin-conjugating | 99.84401 | 137 | 123.637 | 120.3 | 3.08E−01 | 4.61E−01 | 1 | Unchanged Low | |
| | | enzyme E2 | | | | | | | | | |
| P28289 | E08cd2 | tropomodulin | 303.877 | 272 | 123.6484 | 233.0271 | −1.30E+00 | −1.62E−01 | 1 | Unchanged Low | |
| Q15631 | O12ef6 | translin | 173.134 | 152 | 123.6887 | 149.5989 | −4.85E−01 | −1.88E−01 | 1 | Unchanged Low | |
| P49888 | F03cd2 | sulfotransferase; | 122.2586 | 95.1 | 123.7564 | 113.6937 | 1.76E−02 | −3.63E−01 | 1 | Unchanged Low | |
| | | estrogen-prefer | | | | | | | | | |
| P51959 | O12ef5 | cyclin G1 | 116.6841 | 102 | 123.7661 | 114.0381 | 8.50E−02 | −1.99E−01 | 1 | Unchanged Low | |
| Q9Y5V0 | J16ef1 | HSPC038 protein | 114.0123 | 179 | 123.7928 | 139.0584 | 1.19E−01 | 6.54E−01 | 1 | Unchanged Low | |
| P56270 | I12ef1 | MYC-associated zinc | 248.3395 | 140 | 123.8618 | 170.7774 | −1.00E+00 | −8.26E−01 | 1 | Unchanged Low | |
| | | finger prote | | | | | | | | | |
| Q9HD23 | O11gh4 | MRS2-like; | 140.8864 | 117 | 123.8621 | 127.1502 | −1.86E−01 | −2.72E−01 | 1 | Unchanged Low | |
| | | magnesium homeost | | | | | | | | | |
| P78358 | O03ab6 | cancer/testis antigen 1 | 217.9167 | 137 | 123.9307 | 159.681 | −8.14E−01 | −6.68E−01 | 1 | Unchanged Low | |
| O00327 | D24ab2 | aryl hydrocarbon | 118.2008 | 185 | 123.9698 | 142.5298 | 6.87E−02 | 6.50E−01 | 1 | Unchanged Low | |
| | | receptor nuclea | | | | | | | | | |
| Q9H969 | F08gh5 | hypothetical protein | 89.23821 | 177 | 124.0248 | 130.1172 | 4.75E−01 | 9.89E−01 | 1 | Unchanged Low | |
| | | FLJ12969 | | | | | | | | | |
| O15504 | F01cd7 | nucleoporin-like | 200.4449 | 124 | 124.2046 | 149.5876 | −6.90E−01 | −6.92E−01 | 1 | Unchanged Low | |
| | | protein 1 | | | | | | | | | |
| P35250 | I24ef6 | replication factor C | 111.3418 | 112 | 124.3534 | 115.9922 | 1.59E−01 | 1.21E−02 | 1 | Unchanged Low | |
| | | (activator 1) 2 | | | | | | | | | |
| O9BXC0 | F17gh8 | G protein-coupled | 104.4833 | 128 | 124.3568 | 118.786 | 2.51E−01 | 2.87E−01 | 1 | Unchanged Low | |
| | | receptor 81 | | | | | | | | | |
| O15232 | A19ef1 | matrilin 3 | 113.1634 | 110 | 124.4317 | 115.8185 | 1.37E−01 | −4.27E−02 | 1 | Unchanged Low | |
| O76049 | L19cd5 | spectrin SH3 domain | 171.9625 | 299 | 124.5037 | 198.6303 | −4.66E−01 | 8.00E−01 | 1 | Unchanged Low | |
| | | binding pro | | | | | | | | | |
| Q14145 | C21cd8 | Ketch-like ECH- | 193.9671 | 120 | 124.5928 | 146.2645 | −6.39E−01 | −6.90E−01 | 1 | Unchanged Low | |
| | | associated protei | | | | | | | | | |
| P20848 | A10cd1 | serine (or cysteine) | 97.77909 | 161 | 124.6004 | 127.9292 | 3.50E−01 | 7.23E−01 | 1 | Unchanged Low | |
| | | proteinase in | | | | | | | | | |
| Q9P0N6 | C19ef3 | hypothetical protein | 112.0065 | 109 | 124.6034 | 115.304 | 1.54E−01 | −3.53E−02 | 1 | Unchanged Low | |
| | | HSPC242 | | | | | | | | | |
| P05188 | E12ab2 | alkaline phosphatase; | 88.50098 | 164 | 124.6125 | 125.869 | 4.94E−01 | 8.94E−01 | 1 | Unchanged Low | |
| | | placental | | | | | | | | | |
| Q9UBJ4 | A01gh5 | transposon-derived | 131.0923 | 97.3 | 124.7023 | 117.695 | −7.21E−02 | −4.30E−01 | 1 | Unchanged Low | |
| | | Buster1 trans | | | | | | | | | |
| Q13876 | I24ef7 | guiescin Q6 | 144.3305 | 109 | 124.7205 | 125.9829 | −2.11E−01 | −4.06E−01 | 1 | Unchanged Low | |
| Q9UBF2 | F12cd7 | coatomer protein | 110.1673 | 115 | 124.9117 | 116.849 | 1.81E−01 | 6.78E−02 | 1 | Unchanged Low | |
| | | complex; subur | | | | | | | | | |
| P43026 | I23cd3 | growth differentiation | 101.9864 | 152 | 124.9218 | 126.3658 | 2.93E−01 | 5.77E−01 | 1 | Unchanged Low | |
| | | factor 5 (ca | | | | | | | | | |
| Q9HD20 | E14gh4 | CGI-152 protein | 111.6731 | 134 | 124.9301 | 123.6472 | 1.62E−01 | 2.67E−01 | 1 | Unchanged Low | |
| Q96KK6 | P04ab8 | RAB2; member RAS | 119.8171 | 106 | 124.9467 | 116.8445 | 6.05E−02 | −1.80E−01 | 1 | Unchanged Low | |
| | | oncogene fa | | | | | | | | | |
| P40617 | D18ab2 | ADP-ribosylation | 124.1402 | 143 | 125.0573 | 130.7449 | 1.06E−02 | 2.04E−01 | 1 | Unchanged Low | |
| | | factor-like 4 | | | | | | | | | |
| O75651 | L12gh2 | unc-93 homolog A | 131.989 | 128 | 125.0667 | 128.2583 | −7.77E−02 | −4.74E−02 | 1 | Unchanged Low | |
| | | (C. elegans) | | | | | | | | | |
| Q9Y2S1 | L04ef1 | galanin-related peptide | 135.6724 | 134 | 125.067 | 131.4839 | −1.17E−01 | −2.10E−02 | 1 | Unchanged Low | |
| Q13330 | M20ef5 | metastasis associated 1 | 104.9962 | 118 | 125.1131 | 116.0949 | 2.53E−01 | 1.71E−01 | 1 | Unchanged Low | |
| Q9Y389 | L22gh4 | aminoadipate-semi- | 115.3352 | 104 | 125.222 | 114.7055 | 1.19E−01 | −1.55E−01 | 1 | Unchanged Low | |
| | | aldehyde deh | | | | | | | | | |
| O60513 | I03gh1 | UDP-Gal:betaGlcNAc | 113.705 | 106 | 125.2504 | 115.0129 | 1.40E−01 | −1.00E−01 | 1 | Unchanged Low | |
| | | beta 1:4-g | | | | | | | | | |
| O00212 | C15ef6 | ras homolog gene | 407.7018 | 492 | 125.2682 | 341.5176 | −1.70E+00 | 2.70E−01 | 1 | Unchanged Low | |
| | | family: membe | | | | | | | | | |
| Q9NXE1 | J04gh2 | membrane-associated | 96.25484 | 146 | 125.3861 | 122.4125 | 3.81E−01 | 5.97E−01 | 1 | Unchanged Low | |
| | | nucleic ac | | | | | | | | | |
| O95405 | A16cd5 | MAD; mothers | 113.9303 | 223 | 125.3908 | 154.2554 | 1.38E−01 | 9.72E−01 | 1 | Unchanged Low | |
| | | against decapenta | | | | | | | | | |
| P01112 | K12ab7 | v-Ha-ras Harvey rat | 129.7326 | 104 | 125.3947 | 119.6125 | −4.91E−02 | −3.23E−01 | 1 | Unchanged Low | |
| | | sarcoma vira | | | | | | | | | |
| O15120 | F11cd6 | 1-acylglycerol-3- | 264.2959 | 238 | 125.4039 | 209.3705 | −1.08E+00 | −1.49E−01 | 1 | Unchanged Low | |
| | | phosphate O-ac | | | | | | | | | |
| P12645 | N10ab3 | bone morphogenetic protein 3 (o | 107.6548 | 117 | 125.4244 | 116.6372 | 2.20E−01 | 1.18E−01 | 1 | Unchanged Low | |
| P50579 | I01cd7 | methionyl aminopeptidase 2 | 144.5853 | 95.1 | 125.4613 | 121.7158 | −2.05E−01 | −6.04E−01 | 1 | Unchanged Low | |
| O14908 | K01cd6 | regulator of G-protein signalling | 204.527 | 200 | 125.5241 | 176.6683 | −7.04E−01 | −3.26E−02 | 1 | Unchanged Low | |
| Q9HCC6 | I17gh4 | bHLH factor Hes4 | 82.07855 | 213 | 125.5621 | 140.2118 | 6.13E−01 | 1.38E+00 | 1 | Unchanged Low | |
| Q15737 | E23gh1 | suppressor of Ty 6 homolog (S. | 112.2771 | 140 | 125.5917 | 125.8246 | 1.62E−01 | 3.14E−01 | 1 | Unchanged Low | |
| Q9Y519 | A17cd8 | chromosome 22 open reading fra | 100.4037 | 131 | 125.6586 | 119.1056 | 3.24E−01 | 3.87E−01 | 1 | Unchanged Low | |
| Q9Y5J0 | J01ef2 | mitochondrial ribosomal protein | 1117.4353 | 107 | 125.8555 | 116.7986 | 9.99E−02 | −1.33E−01 | 1 | Unchanged Low | |
| P51687 | O16cd1 | sulfite oxidase | 105.2314 | 128 | 125.9497 | 119.5637 | 2.59E−01 | 2.77E−01 | 1 | Unchanged Low | |
| P51178 | A13ef6 | phospholipase C; delta 1 | 126.4221 | 162 | 126.0345 | 138.0904 | −4.43E−03 | 3.56E−01 | 1 | Unchanged Low | |
| Q9H8J6 | H23gh5 | NDRG family member 3 | 121.7115 | 198 | 126.048 | 148.4766 | 5.05E−02 | 7.00E−01 | 1 | Unchanged Low | |
| O95900 | B20ef8 | hypothetical protein CLONE2492 | 165.6404 | 257 | 126.0637 | 182.738 | −3.94E−01 | 6.31E−01 | 1 | Unchanged Low | |
| Q9Y6N3 | B01cd5 | chloride channel; calcium activat | 99.89597 | 135 | 126.1695 | 120.3564 | 3.37E−01 | 4.35E−01 | 1 | Unchanged Low | |
| Q07869 | C21cd1 | peroxisome proliferative activate | 120.1031 | 112 | 126.2127 | 119.4872 | 7.15E−02 | −9.89E−02 | 1 | Unchanged Low | |
| Q9Y333 | I07gh4 | chromosome 6 open reading fra | 109.0761 | 114 | 126.2563 | 116.4501 | 2.11E−01 | 6.39E−02 | 1 | Unchanged Low | |
| Q9NPD1 | L06gh2 | G protein-coupled receptor 85 | 206.0578 | 285 | 126.2683 | 205.7432 | −7.07E−01 | 4.67E−01 | 1 | Unchanged Low | |
| Q15928 | K06cd3 | zinc finger protein 141 (clone pH | 101.1559 | 188 | 126.2732 | 138.6304 | 3.20E−01 | 8.98E−01 | 1 | Unchanged Low | |
| O43826 | I04ab4 | glucose-6-phosphatase; transpo | 229.7084 | 111 | 126.3113 | 155.5451 | −8.63E−01 | −1.05E+00 | 1 | Unchanged Low | |
| Q9NWL0 | J05gh2 | hypothetical protein FLJ20758 | 114.0453 | 120 | 126.3891 | 120.1186 | 1.48E−01 | 7.25E−02 | 1 | Unchanged Low | |
| P31629 | B04ef6 | human immunodeficiency virus ty | 116.0174 | 211 | 126.4284 | 151.1911 | 1.24E−01 | 8.64E−01 | 1 | Unchanged Low | |
| O15530 | I10ab8 | 3-phosphoinosilide dependent p | 186.1234 | 193 | 126.45 | 168.488 | −5.58E−01 | 5.15E−02 | 1 | Unchanged Low | |
| Q9Y2R5 | L21ef4 | mitochondrial ribosomal protein S | 1106.3741 | 136 | 126.4713 | 122.8701 | 2.50E−01 | 3.52E−01 | 1 | Unchanged Low | |
| Q15208 | D01cd7 | serine/threonine kinase 38 | 158.8765 | 204 | 126.5253 | 163.2891 | −3.28E−01 | 3.64E−01 | 1 | Unchanged Low | |
| Q9UET6 | J19gh6 | FtsJ homolog 1 (E. coli) | 102.5266 | 120 | 126.5378 | 116.4386 | 3.04E−01 | 2.30E−01 | 1 | Unchanged Low | |
| Q9H5N0 | F15gh5 | chromosome 7 open reading fra | 102.3349 | 129 | 126.5687 | 119.1911 | 3.07E−01 | 3.30E−01 | 1 | Unchanged Low | |
| Q9H019 | B22gh4 | hypothetical protein RP1-317E23 | 102.5329 | 150 | 126.5974 | 126.5367 | 3.04E−01 | 5.53E−01 | 1 | Unchanged Low | |
| Q9Y673 | F05cd8 | Alg5; S. cerevisiae; homolog of | 100.2543 | 131 | 126.6884 | 119.1797 | 3.38E−01 | 3.81E−01 | 1 | Unchanged Low | |
| O75690 | M08gh7 | UHS KerB | 121.3598 | 150 | 126.7819 | 132.5659 | 6.31E−02 | 3.01E−01 | 1 | Unchanged Low | |
| Q9NUP6 | H22gh2 | hypothetical protein FLJ11220 | 95.908 | 144 | 126.8164 | 122.1684 | 4.03E−01 | 5.84E−01 | 1 | Unchanged Low | |
| Q14296 | C03cd7 | FAST kinase | 122.4924 | 92.9 | 126.9021 | 114.1093 | 5.10E−02 | −3.98E−01 | 1 | Unchanged Low | |
| Q9NW55 | B20gh2 | hypothetical protein FLJ10307 | 94.73914 | 141 | 126.9877 | 120.8058 | 4.23E−01 | 5.70E−01 | 1 | Unchanged Low | |
| O43639 | H01cd3 | NCK adaptor protein 2 | 139.9609 | 79.6 | 127.0293 | 115.5457 | −1.40E−01 | −8.13E−01 | 1 | Unchanged Low | |
| P17544 | L08ab2 | activating transcription factor 7 | 108.5839 | 112 | 127.0848 | 115.9726 | 2.27E−01 | 4.79E−02 | 1 | Unchanged Low | |
| O75911 | M01cd5 | short-chain dehydrogenase/redu | 156.3658 | 173 | 127.1125 | 152.0313 | −2.99E−01 | 1.43E−01 | 1 | Unchanged Low | |
| Q06430 | K15ef3 | glucosaminyl (N-acetyl) transfera | 94.56995 | 139 | 127.1541 | 120.315 | 4.27E−01 | 5.58E−01 | 1 | Unchanged Low | |
| Q13769 | D10cd3 | chromosome 22 open reading fra | 122.7812 | 121 | 127.1547 | 123.5081 | 5.05E−02 | −2.60E−02 | 1 | Unchanged Low | |
| Q9H864 | J14gh5 | hypothetical protein FLJ11021 si | 97.58044 | 142 | 127.1909 | 122.387 | 3.82E−01 | 5.45E−01 | 1 | Unchanged Low | |
| O95500 | F06cd7 | claudin 14 | 183.0294 | 181 | 127.2815 | 163.6478 | −5.24E−01 | −1.90E−02 | 1 | Unchanged Low | |
| P14222 | P02cd2 | perforin 1 (pore forming protein) | 99.11893 | 135 | 127.3674 | 120.5243 | 3.52E−01 | 4.47E−01 | 1 | Unchanged Low | |
| Q9Y4C8 | B06ef2 | KIAA0682 gene product | 105.4769 | 146 | 127.3803 | 126.2296 | 2.72E−01 | 4.67E−01 | 1 | Unchanged Low | |
| Q9H3H5 | M23ab4 | dolichyl-phosphate (UDP-N-acety | 193.8179 | 108 | 127.5134 | 143.2265 | −6.04E−01 | −8.39E−01 | 1 | Unchanged Low | |
| Q13433 | G05cd8 | LIV-1 protein; estrogen regulate | 118.095 | 105 | 127.6623 | 116.7563 | 1.12E−01 | −1.76E−01 | 1 | Unchanged Low | |
| Q00059 | O07cd2 | transcription factor A; mitochond | 111.4615 | 131 | 127.6629 | 123.3486 | 1.96E−01 | 2.32E−01 | 1 | Unchanged Low | |
| Q15119 | I06ab8 | pyruvate dehydrogenase kinase; | 97.01115 | 135 | 127.6675 | 120.0132 | 3.96E−01 | 4.81E−01 | 1 | Unchanged Low | |
| O43791 | F03cd3 | speckle-type POZ protein | 111.349 | 114 | 127.7061 | 117.7111 | 1.98E−01 | 3.49E−02 | 1 | Unchanged Low | |
| Q14206 | P10cd5 | Down syndrome critical region ge | 119.8147 | 263 | 127.7164 | 170.2393 | 9.21E−02 | 1.14E+00 | 1 | Unchanged Low | |
| Q92926 | N24cd1 | SWI/SNF related; matrix associa | 115.147 | 105 | 127.7503 | 116.1179 | 1.50E−01 | −1.27E−01 | 1 | Unchanged Low | |
| Q9P288 | G15ef3 | BRCA2 and CDKN1A interacting | 148.9477 | 132 | 127.8623 | 136.3262 | −2.20E−01 | −1.72E−01 | 1 | Unchanged Low | |
| Q9NZU5 | C16ef4 | LIM and cysteine-rich domains 1 | 164.1438 | 128 | 127.9349 | 139.9549 | −3.60E−01 | −3.61E−01 | 1 | Unchanged Low | |
| Q13569 | O13cd2 | thymine-DNA glycosylase | 83.16502 | 172 | 127.9545 | 127.6936 | 6.22E−01 | 1.05E+00 | 1 | Unchanged Low | |
| P48167 | D17ef6 | glycine receptor; beta | 109.5501 | 139 | 127.9738 | 125.3664 | 2.24E−01 | 3.39E−01 | 1 | Unchanged Low | |
| Q9NQR1 | I02gh4 | PR/SET domain containing prote | 154.2185 | 92.7 | 128.0258 | 124.9651 | −2.69E−01 | −7.35E−01 | 1 | Unchanged Low | |
| O43405 | B23ab4 | coagulation factor C homolog; cc | 103.0764 | 121 | 128.1793 | 117.417 | 3.14E−01 | 2.31E−01 | 1 | Unchanged Low | |
| Q9P028 | N24ef2 | CCR4-NOT transcription comple | 104.0341 | 133 | 128.2089 | 121.9053 | 3.01E−01 | 3.59E−01 | 1 | Unchanged Low | |
| Q9Y6E8 | P04cd7 | sirtuin silent mating type informal | 109.1684 | 110 | 128.2444 | 115.6628 | 2.32E−01 | 5.37E−03 | 1 | Unchanged Low | |
| Q9NV65 | H17gh3 | hypothetical protein FLJ10901 | 92.33747 | 188 | 128.3243 | 136.3658 | 4.75E−01 | 1.03E+00 | 1 | Unchanged Low | |
| Q9H0R8 | M13gh7 | GABA(A) receptor-associated pr | 122.091 | 179 | 128.3386 | 143.1503 | 7.20E−02 | 5.52E−01 | 1 | Unchanged Low | |
| O95817 | P02cd4 | BCL2-associated athanogene 3 | 107.1752 | 114 | 128.4319 | 116.5745 | 2.61E−01 | 9.05E−02 | 1 | Unchanged Low | |
| P36956 | K10cd2 | sterol regulatory element binding | 140.2284 | 109 | 128.532 | 125.9988 | −1.26E−01 | −3.60E−01 | 1 | Unchanged Low | |
| P08236 | D05ab3 | glucuronidase; beta | 123.7102 | 114 | 128.6103 | 122.0379 | 5.60E−02 | −1.21E−01 | 1 | Unchanged Low | |
| Q13425 | D12cd2 | syntrophin; beta 2 (dystrophin-as | 106.7241 | 122 | 128.6723 | 119.2218 | 2.70E−01 | 1.96E−01 | 1 | Unchanged Low | |
| Q14676 | E18gh1 | KIAA0170 gene product | 93.41288 | 145 | 128.7328 | 122.2375 | 4.83E−01 | 6.30E−01 | 1 | Unchanged Low | |
| P16106 | O12gh6 | H3 histone family; member F | 98.02235 | 136 | 128.7695 | 120.7804 | 3.94E−01 | 4.68E−01 | 1 | Unchanged Low | |
| Q14773 | J03ab6 | intercellular adhesion molecule 4 | 101.85 | 137 | 128.8098 | 122.427 | 3.39E−01 | 4.24E−01 | 1 | Unchanged Low | |
| O14736 | N19gh4 | putative methyltransferase | 124.8741 | 126 | 128.8151 | 126.6152 | 4.48E−02 | 1.47E−02 | 1 | Unchanged Low | |
| Q9Y297 | N21cd4 | beta-transducin repeat containing | 152.2864 | 142 | 128.8641 | 141.0555 | −2.41E−01 | −1.01E−01 | 1 | Unchanged Low | |
| Q13585 | H16cd4 | G protein-coupled receptor 50 | 121.9424 | 113 | 128.8853 | 121.1757 | 7.99E−02 | −1.14E−01 | 1 | Unchanged Low | |
| O60858 | L24cd5 | ret finger protein 2 | 110.1643 | 110 | 128.8939 | 116.2529 | 2.27E−01 | −6.09E−03 | 1 | Unchanged Low | |
| Q9Y2Z6 | E06ef2 | CGI-07 protein | 96.22816 | 139 | 128.9272 | 121.3443 | 4.22E−01 | 5.29E−01 | 1 | Unchanged Low | |
| Q9UG66 | P16ef7 | hypothetical protein DKFZp586G | 318.3297 | 374 | 128.9795 | 273.7118 | −1.30E+00 | 2.32E−01 | 1 | Unchanged Low | |
| Q9Y6G5 | N17ef2 | PTD002 protein | 107.7937 | 141 | 129.1324 | 125.8408 | 2.61E−01 | 3.83E−01 | 1 | Unchanged Low | |
| P98155 | E06cd3 | very low density lipoprotein rece | 113.1365 | 104 | 129.1355 | 115.483 | 1.91E−01 | −1.19E−01 | 1 | Unchanged Low | |
| Q9NP50 | C07gh5 | TERA protein | 94.63638 | 199 | 129.2792 | 140.8474 | 4.50E−01 | 1.07E+00 | 1 | Unchanged Low | |
| P28328 | C01ab8 | peroxisomal membrane protein 3 | 321.6292 | 219 | 129.3474 | 223.2274 | −1.31E+00 | −5.S6E−01 | 1 | Unchanged Low | |
| Q969T4 | M24cd6 | ubiquitin-conjugating enzyme E2 | 117.2071 | 100 | 129.3512 | 11S.6428 | 1.42E−01 | −2.24E−01 | 1 | Unchanged Low | |
| P19827 | D18ab6 | inter-alpha (globulin) inhibitor; H | 239.4455 | 186 | 129.3613 | 185.0551 | −8.88E−01 | −3.62E−01 | 1 | Unchanged Low | |
| O15096 | M18ab8 | phosphatidylinositol 4-kinase; ca | 117.2574 | 115 | 129.4365 | 120.4316 | 1.43E−01 | −3.31E−02 | 1 | Unchanged Low | |
| Q9Y5J6 | J16cd7 | fracture callus 1 homolog (rat) | 102.357 | 127 | 129.4503 | 119.4401 | 3.39E−01 | 3.06E−01 | 1 | Unchanged Low | |
| Q92889 | M24ab4 | excision repair cross-complemen | 157.5735 | 97.6 | 129.5255 | 128.238 | −2.83E−01 | −6.91E−01 | 1 | Unchanged Low | |
| P08697 | C07ef1 | serine (or cysteine) proteinase in | 138.5253 | 136 | 129.5425 | 134.8048 | −9.67E−02 | −2.29E−02 | 1 | Unchanged Low | |
| Q9UBX1 | A05cd4 | cathepsin F | 113.4065 | 118 | 129.6557 | 120.4495 | 1.93E−01 | 6.08E−02 | 1 | Unchanged Low | |
| Q9NP17 | D22ef1 | hypothetical protein LOC51315 | 240.5378 | 171 | 129.76 | 180.4148 | −8.90E−01 | −4.93E−01 | 1 | Unchanged Low | |
| P11586 | D14ef7 | methylenetetrahydrofolate dehyd | 141.2456 | 106 | 129.8444 | 125.7751 | −1.21E−01 | −4.11E−01 | 1 | Unchanged Low | |
| O96010 | J15cd4 | LIM domain binding 1 | 90.9506 | 155 | 129.8567 | 125.1912 | 5.14E−01 | 7.67E−01 | 1 | Unchanged Low | |
| Q13188 | P13cd2 | serine/threonine kinase 3 (STE2 | 117.4664 | 135 | 129.8809 | 127.4091 | 1.45E−01 | 1.99E−01 | 1 | Unchanged Low | |
| Q13845 | I21ab3 | B-cell CLL/lymphoma 7B | 145.9359 | 66.3 | 129.9273 | 121.3988 | −1.68E−01 | −7.24E−01 | 1 | Unchanged Low | |
| Q9Y5W9 | D07cd8 | sorting nexin 11 | 144.6605 | 198 | 130.0152 | 157.7019 | −1.54E−01 | 4.56E−01 | 1 | Unchanged Low | |
| O60539 | I02cd7 | Ras association (RalGDS/AF-6) | 126.8466 | 102 | 130.0346 | 119.7412 | 3.58E−02 | −3.10E−01 | 1 | Unchanged Low | |
| O60885 | I02ef3 | bromodomain-containing 4 | 114.9928 | 98.1 | 130.1093 | 114.4111 | 1.78E−01 | −2.29E−01 | 1 | Unchanged Low | |
| Q9H2B0 | O16gh5 | nucleosome assembly protein 1-I | 122.5782 | 147 | 130.1112 | 133.1825 | 8.60E−02 | 2.61E−01 | 1 | Unchanged Low | |
| P51582 | C24ab8 | pyrimidinergic receptor P2Y; G-p | 116.4263 | 109 | 130.158 | 118.649 | 1.61E−01 | −9.03E−02 | 1 | Unchanged Low | |
| O00585 | F08cd1 | smalt inducible cytokine subfamil | 168.745 | 216 | 130.1849 | 171.5056 | −3.74E−01 | 3.53E−01 | 1 | Unchanged Low | |
| Q15131 | B23ef5 | cyclin-dependent kinase (CDC2- | 168.6104 | 108 | 130.2201 | 135.7223 | −3.73E−01 | −6.38E−01 | 1 | Unchanged Low | |
| O75148 | F08cd8 | cryptochrome 2 (photolyase-like) | 86.14249 | 172 | 130.27 | 129.5265 | 5.97E−01 | 9.99E−01 | 1 | Unchanged Low | |
| Q15691 | G17cd8 | microtubule-associated protein: | 173.7024 | 168 | 130.2758 | 157.4437 | −4.15E−01 | −4.51E−02 | 1 | Unchanged Low | |
| Q15779 | H21ab8 | procollagen (type III) N-endopep | 141.4408 | 133 | 130.2905 | 135.0227 | −1.18E−01 | −8.51E−02 | 1 | Unchanged Low | |
| Q9UHD2 | K14cd8 | TANK-binding kinase 1 | 82.31867 | 172 | 130.2947 | 128.1679 | 6.62E−01 | 1.06E+00 | 1 | Unchanged Low | |
| Q9NSS3 | K15gh3 | hypothetical protein DKFZp434E | 107.5389 | 115 | 130.3095 | 117.5518 | 2.77E−01 | 9.44E−02 | 1 | Unchanged Low | |
| Q13797 | I01ef7 | integrin: alpha 9 | 156.9946 | 65 | 130.4752 | 124.1726 | −2.67E−01 | −8.84E−01 | 1 | Unchanged Low | |
| P46531 | M10ef5 | Notch homolog 1: translocation-a | 86.01963 | 164 | 130.4897 | 126.8111 | 6.01E−01 | 9.30E−01 | 1 | Unchanged Low | |
| O43262 | C01cd6 | deleted in lymphocytic leukemia: | 76.79463 | 195 | 130.4943 | 134.2594 | 7.65E−01 | 1.35E+00 | 1 | Unchanged Low | |
| Q9Y2H6 | K12ef8 | KIAA0970 protein | 170.8655 | 128 | 130.5238 | 142.9858 | −3.89E−01 | −4.22E−01 | 1 | Unchanged Low | |
| P25788 | J11ab8 | proteasome (prosome: macropai | 256.6025 | 285 | 130.5899 | 224.7239 | −9.86E−01 | 1.40E−01 | 1 | Unchanged Low | |
| Q9BQS8 | D09gh7 | FYVE and coiled-coil domain co | 164.6348 | 207 | 130.6451 | 167.5571 | −3.34E−01 | 3.33E−01 | 1 | Unchanged Low | |
| Q9Y396 | J04ef1 | elongation of very long chain fatt | 162.6431 | 82.2 | 130.6722 | 125.1558 | −3.16E−01 | −9.85E−01 | 1 | Unchanged Low | |
| Q9P0R7 | K06ef2 | hypothetical protein LOC51242 | 126.7302 | 218 | 130.6804 | 158.5209 | 4.43E−02 | 7.84E−01 | 1 | Unchanged Low | |
| O15269 | F17cd6 | serine palmitoyltransferase: long | 97.42808 | 133 | 130.7429 | 120.3129 | 4.24E−01 | 4.46E−01 | 1 | Unchanged Low | |
| Q9UFX0 | B02ef2 | calcium binding atopy-related au | 233.5089 | 287 | 130.8943 | 217.1357 | −8.35E−01 | 2.98E−01 | 1 | Unchanged Low | |
| Q9H663 | F24gh5 | likely ortholog of mouse actin-rel | 98.1679 | 128 | 130.9258 | 119.1698 | 4.15E−01 | 3.87E−01 | 1 | Unchanged Low | |
| O75410 | P17cd2 | transforming; acidic coiled-coil c | 224.1188 | 133 | 130.9827 | 162.703 | −7.75E−01 | −7.53E−01 | 1 | Unchanged Low | |
| Q9Y5N5 | I14cd8 | putative N6-DNA-methyltransfera | 99.34841 | 133 | 131.0924 | 121.2285 | 4.00E−01 | 4.24E−01 | 1 | Unchanged Low | |
| Q9BQ72 | F02gh7 | hypothetical protein MGC10471 | 106.3824 | 115 | 131.1126 | 117.4135 | 3.02E−01 | 1.09E−01 | 1 | Unchanged Low | |
| Q9Y303 | E10ef2 | CGI-14 protein | 153.5787 | 101 | 131.2035 | 128.5162 | −2.27E−01 | −6.08E−01 | 1 | Unchanged Low | |
| Q9P005 | A22ef8 | HSPC159 protein | 341.3063 | 579 | 131.3146 | 350.7027 | −1.38E+00 | 7.64E−01 | 1 | Unchanged Low | |
| Q9Y3A1 | H19ef2 | DKFZP566O084 protein | 200.4927 | 262 | 131.514 | 198.1031 | −6.08E−01 | 3.88E−01 | 1 | Unchanged Low | |
| Q15172 | C12cd1 | protein phosphatase 2: regulator | 168.3914 | 140 | 131.5524 | 146.553 | −3.56E−01 | −2.69E−01 | 1 | Unchanged Low | |
| Q9BV19 | I19gh6 | hypothetical protein MGC955 | 96.47316 | 141 | 131.6188 | 122.8739 | 4.48E−01 | 5.43E−01 | 1 | Unchanged Low | |
| Q14534 | E11cd2 | squalene epoxidase | 155.5799 | 71.3 | 131.8107 | 119.574 | −2.39E−01 | −1.13E+00 | 1 | Unchanged Low | |
| Q9BS43 | G19gh8 | hypothetical protein MGC12435 | 592.1694 | 121 | 131.9022 | 281.8569 | −2.17E+00 | −2.29E+00 | 1 | Unchanged Low | |
| P49788 | D24ab8 | retinoic acid receptor responder | 97.19637 | 135 | 131.9355 | 121.3412 | 4.41E−01 | 4.73E−01 | 1 | Unchanged Low | |
| Q9UP83 | M10cd6 | component of oligomeric golgi c | 100.2446 | 141 | 131.936 | 124.3058 | 3.96E−01 | 4.89E−01 | 1 | Unchanged Low | |
| Q9Y2X0 | N11cd5 | thyroid hormone receptor-associa | 116.8962 | 97.9 | 131.9541 | 115.5741 | 1.75E−01 | −2.56E−01 | 1 | Unchanged Low | |
| Q9Y3E3 | I15ef2 | CGI-145 protein | 94.76733 | 132 | 131.9919 | 119.5355 | 4.78E−01 | 4.76E−01 | 1 | Unchanged Low | |
| O95183 | J14cd6 | vesicle-associated membrane pr | 91.19765 | 157 | 132.0539 | 126.6473 | 5.34E−01 | 7.81E−01 | 1 | Unchanged Low | |
| Q13242 | N22cd3 | splicing factor: arginine/serine-ri | 156.0624 | 141 | 132.0637 | 143.0016 | −2.41E−01 | −1.48E−01 | 1 | Unchanged Low | |
| Q9Y5R5 | A06gh1 | doublesex and mab-3 related tra | 100.6446 | 162 | 132.1528 | 131.5571 | 3.90E−01 | 6.81E−01 | 1 | Unchanged Low | |
| P04181 | N02ab7 | ornithine aminotransferase (gyra | 112.3954 | 106 | 132.1687 | 116.724 | 2.34E−01 | −8.99E−02 | 1 | Unchanged Low | |
| Q14254 | B15ab4 | flotillin 2 | 130.1586 | 154 | 132.2038 | 138.6301 | 2.25E−02 | 2.38E−01 | 1 | Unchanged Low | |
| Q9Y6E6 | P08cd7 | sirtuin silent mating type informa | 94.40989 | 136 | 132.2501 | 121.0265 | 4.86E−01 | 5.31E−01 | 1 | Unchanged Low | |
| Q9H237 | D20gh5 | porcupine | 148.2204 | 258 | 132.2856 | 179.3923 | −1.64E−01 | 7.98E−01 | 1 | Unchanged Low | |
| Q92785 | N21cd2 | requiem: apoptosis response zin | 118.3718 | 108 | 132.3893 | 119.7533 | 1.61E−01 | −1.26E−01 | 1 | Unchanged Low | |
| O95458 | N11cd2 | tubulin-specific chaperone d | 128.9522 | 100 | 132.4761 | 120.4814 | 3.89E−02 | −3.67E−01 | 1 | Unchanged Low | |
| P20749 | P23ef6 | B-cell CLL/lymphoma 3 | 264.1174 | 186 | 132.484 | 194.2507 | −9.95E−01 | −5.05E−01 | 1 | Unchanged Low | |
| Q9H2N8 | D24gh7 | hypothetical protein GL012 | 104.3233 | 160 | 132.5137 | 132.1507 | 3.45E−01 | 6.14E−01 | 1 | Unchanged Low | |
| Q15020 | M08gh1 | squamous cell carcinoma antige | 117.2757 | 147 | 132.5393 | 132.2727 | 1.77E−01 | 3.26E−01 | 1 | Unchanged Low | |
| P42025 | O01gh1 | ARP1 actin-related protein 1 ho | 243.7979 | 92 | 132.554 | 156.1129 | −8.79E−01 | −1.41E+00 | 1 | Unchanged Low | |
| P98173 | I23gh5 | 2.19 gene | 113.0426 | 116 | 132.6703 | 120.727 | 2.31E−01 | 4.31E−02 | 1 | Unchanged Low | |
| O95295 | C06cd8 | SNARE associated protein snapi | 324.3312 | 119 | 132.6986 | 192.0882 | −1.29E+00 | −1.44E+00 | 1 | Unchanged Low | |
| Q9NVA1 | F15gh3 | chromosome 20 open reading fra | 111.7508 | 100 | 132.7232 | 114.8529 | 2.48E−01 | −1.59E−01 | 1 | Unchanged Low | |
| P35573 | O03ab2 | amylo-1: 6-glucosidase: 4-alpha- | 111.9583 | 106 | 132.7432 | 116.6065 | 2.46E−01 | −8.27E−02 | 1 | Unchanged Low | |
| Q13126 | K13ab7 | methylthioadenosine phosphoryl | 202.8169 | 236 | 132.9211 | 190.4847 | −6.10E−01 | 2.17E−01 | 1 | Unchanged Low | |
| P43034 | C11ab8 | platelet-activating factor acetylhy | 106.8174 | 125 | 132.987 | 121.4511 | 3.16E−01 | 2.22E−01 | 1 | Unchanged Low | |
| Q01844 | E15ab5 | Ewing sarcoma breakpoint regio | 111.2628 | 102 | 133.001 | 115.5021 | 2.57E−01 | −1.22E−01 | 1 | Unchanged Low | |
| P27482 | E18ab6 | calmodulin-like 3 | 114.7017 | 104 | 133.018 | 117.2725 | 2.14E−01 | −1.40E−01 | 1 | Unchanged Low | |
| Q9NUW4 | N23gh3 | BRIX | 223.8591 | 77.1 | 133.0208 | 144.6673 | −7.51E−01 | −1.54E+00 | 1 | Unchanged Low | |
| P78552 | J23ab6 | interleukin 13 receptor: alpha 1 | 118.2476 | 93.9 | 133.0765 | 115.0749 | 1.70E−01 | −3.33E−01 | 1 | Unchanged Low | |
| P51843 | O24ab2 | nuclear receptor subfamily 0: gro | 104.7329 | 112 | 133.1401 | 116.7757 | 3.46E−01 | 1.03E−01 | 1 | Unchanged Low | |
| P15822 | L23ab5 | human immunodeficiency virus ty | 83.26942 | 160 | 133.2118 | 125.3978 | 6.78E−01 | 9.40E−01 | 1 | Unchanged Low | |
| Q9BXY9 | O13gh8 | RALBP1 associated Eps domain | 90.84238 | 140 | 133.274 | 121.2581 | 5.53E−01 | 6.20E−01 | 1 | Unchanged Low | |
| Q9ULB4 | P10ef1 | cadherin 9: type 2 (T1-cadherin) | 275.2974 | 137 | 133.4641 | 181.8847 | −1.04E+00 | −1.01E+00 | 1 | Unchanged Low | |
| Q9NZZ7 | C08ef8 | HSPC171 protein | 214.9523 | 265 | 133.5412 | 204.594 | −6.87E−01 | 3.04E−01 | 1 | Unchanged Low | |
| Q02086 | C09cd2 | Sp2 transcription factor | 101.0989 | 133 | 133.5465 | 122.3826 | 4.02E−01 | 3.90E−01 | 1 | Unchanged Low | |
| Q9BUV9 | H14gh6 | DKFZP586J0119 protein | 102.1174 | 119 | 133.5923 | 118.2651 | 3.88E−01 | 2.22E−01 | 1 | Unchanged Low | |
| Q9UHJ9 | A09ef4 | FGF receptor activating protein 1 | 100.7903 | 121 | 133.6008 | 118.5627 | 4.07E−01 | 2.67E−01 | 1 | Unchanged Low | |
| Q16401 | E11cd1 | proteasome (prosome: macropai | 143.6091 | 120 | 133.6156 | 132.2946 | −1.04E−01 | −2.63E−01 | 1 | Unchanged Low | |
| Q9Y4K4 | D04cd6 | mitogen-activated protein kinase | 132.4072 | 158 | 133.6676 | 141.2069 | 1.37E−02 | 2.51E−01 | 1 | Unchanged Low | |
| O60463 | H06cd3 | phosphatidic acid phosphatase ty | 103.2417 | 151 | 133.6898 | 129.2479 | 3.73E−01 | 5.47E−01 | 1 | Unchanged Low | |
| O95639 | P20cd6 | cleavage and polyadenylation sp | 183.1762 | 67.6 | 133.7241 | 128.1539 | −4.54E−01 | −1.44E+00 | 1 | Unchanged Low | |
| P50539 | N19ab7 | MAX interacting protein 1 | 175.6646 | 187 | 133.7304 | 165.4016 | −3.93E−01 | 8.87E−02 | 1 | Unchanged Low | |
| Q9UH62 | I15ef3 | ALEX3 protein | 118.2527 | 171 | 133.7314 | 140.9912 | 1.77E−01 | 5.32E−01 | 1 | Unchanged Low | |
| O15525 | C13ab7 | v-maf musculoaponeurotic fibros | 105.3252 | 109 | 133.7521 | 116.1167 | 3.45E−01 | 5.31E−02 | 1 | Unchanged Low | |
| Q9H8T0 | H21gh5 | fused toes homolog (mouse) | 106.2145 | 241 | 133.7606 | 160.2791 | 3.33E−01 | 1.18E+00 | 1 | Unchanged Low | |
| Q9Y4X5 | K03cd6 | ariadne homolog: ubiquitin-conju | 99.46477 | 128 | 133.8771 | 120.4976 | 4.29E−01 | 3.66E−01 | 1 | Unchanged Low | |
| Q9H9E1 | O01gh5 | ankyrin repeat: family A (RFXAN | 172.6266 | 78.8 | 134.052 | 128.4541 | −3.65E−01 | −1.13E+00 | 1 | Unchanged Low | |
| Q93063 | F13ab3 | exostoses (multiple) 2 | 123.2886 | 161 | 134.3466 | 139.4501 | 1.24E−01 | 3.82E−01 | 1 | Unchanged Low | |
| Q9H992 | D22gh5 | axotrophin | 59.54768 | 149 | 134.4784 | 124.4411 | 5.87E−01 | 7.37E−01 | 1 | Unchanged Low | |
| O43237 | P02ab4 | dynein: cytoplasmic; light interme | 290.6769 | 119 | 134.4914 | 181.4903 | −1.11E+00 | −1.28E+00 | 1 | Unchanged Low | |
| Q99643 | H02cd1 | succinate dehydrogenase compl | 143.4474 | 125 | 134.5527 | 134.443 | −9.24E−02 | −1.95E−01 | 1 | Unchanged Low | |
| P24386 | F11ef1 | choroideremia (Rab escort protei | 93.86058 | 129 | 134.5538 | 119.0446 | 5.20E−01 | 4.56E−01 | 1 | Unchanged Low | |
| Q9Y5F7 | F12gh4 | protocadherin gamma subfamily | 101.7878 | 114 | 134.5585 | 116.632 | 4.03E−01 | 1.58E−01 | 1 | Unchanged Low | |
| P06756 | D14ab6 | integrin: alpha V (vitronectin rec | 200.0782 | 219 | 134.6115 | 184.6891 | −5.72E−01 | 1.33E−01 | 1 | Unchanged Low | |
| Q9H5L7 | C02gh6 | chromosome 1 open reading frar | 173.8106 | 148 | 134.6601 | 152.0822 | −3.68E−01 | −2.34E−01 | 1 | Unchanged Low | |
| O15517 | A14ef6 | tumor necrosis factor receptor su | 92.33364 | 207 | 134.6603 | 144.7566 | 5.44E−01 | 1.17E+00 | 1 | Unchanged Low | |
| Q9P0N5 | K24ef2 | HSPC244 | 107.9991 | 129 | 134.6618 | 123.7736 | 3.18E−01 | 2.53E−01 | 1 | Unchanged Low | |
| Q9NWT2 | D19gh2 | hypothetical protein FLJ20623 | 107.8864 | 105 | 134.7112 | 115.8334 | 3.20E−01 | −4.05E−02 | 1 | Unchanged Low | |
| Q9Y2A0 | O02cd7 | TP53 target gene 1 | 127.408 | 88.2 | 134.8491 | 116.8334 | 8.19E−02 | −5.30E−01 | 1 | Unchanged Low | |
| Q9NZ43 | N16gh4 | uncharacterized hematopoietic s | 141.2528 | 137 | 134.9524 | 137.7883 | −6.58E−02 | −4.24E−02 | 1 | Unchanged Low | |
| P27986 | A10ef1 | phosphoinositide-3-kinase: regul | 111.3806 | 112 | 134.9586 | 119.3503 | 2.77E−01 | 4.28E−03 | 1 | Unchanged Low | |
| Q9UBP6 | J13ef1 | methyltransferase-like 1 | 107.2187 | 122 | 134.986 | 121.358 | 3.32E−01 | 1.85E−01 | 1 | Unchanged Low | |
| Q9BWK5 | P24gh5 | hypothetical protein MGC5242 | 101.1324 | 123 | 134.9881 | 119.8654 | 4.17E−01 | 2.88E−01 | 1 | Unchanged Low | |
| P54802 | M24ab6 | N-acetylglucosaminidase: alpha- | 206.038 | 66.3 | 135.077 | 135.7985 | −6.09E−01 | −1.64E+00 | 1 | Unchanged Low | |
| O75963 | H09cd7 | G-protein coupled receptor | 110.3886 | 184 | 135.3412 | 143.3711 | 2.94E−01 | 7.40E−01 | 1 | Unchanged Low | |
| Q9NXZ4 | F03ef4 | ELG protein | 124.5205 | 105 | 135.3934 | 121.5448 | 1.21E−01 | −2.50E−01 | 1 | Unchanged Low | |
| O60353 | B11cd3 | frizzled homolog 6 (Drosophila) | 85.64918 | 146 | 135.5169 | 122.5147 | 6.62E−01 | 7.73E−01 | 1 | Unchanged Low | |
| Q9NQS6 | F13gh4 | latexin protein | 94.21522 | 163 | 135.5761 | 130.917 | 5.25E−01 | 7.90E−01 | 1 | Unchanged Low | |
| O14776 | A19cd7 | transcription elongation regulato | 113.3803 | 117 | 135.6603 | 122.1137 | 2.59E−01 | 4.90E−02 | 1 | Unchanged Low | |
| Q9Y5A2 | L13ef1 | putative zinc finger protein NY-R | 154.0704 | 186 | 135.6708 | 158.5684 | −1.83E−01 | 2.71E−01 | 1 | Unchanged Low | |
| P49842 | G23cd6 | serine/threonine kinase 19 | 133.3557 | 171 | 135.7013 | 146.5953 | 2.52E−02 | 3.56E−01 | 1 | Unchanged Low | |
| Q06546 | J14ab5 | GA binding protein transcription I | 88.93312 | 138 | 135.7488 | 120.885 | 6.10E−01 | 6.34E−01 | 1 | Unchanged Low | |
| Q9Y2E7 | I02ef8 | KIAA0938 protein | 109.8218 | 105 | 135.7763 | 116.730 | 3.06E−01 | −7.00E−02 | 1 | Unchanged Low | |
| Q9UM11 | I02ef2 | Fzr1 protein | 80.82847 | 186 | 135.8949 | 134.12 | 7.50E−01 | 1.20E+00 | 1 | Unchanged Low | |
| Q99447 | B14ab8 | phosphate cytidylyltransferase 2: | 174.8201 | 77.4 | 135.988 | 129.39 | −3.62E−01 | −1.18E+00 | 1 | Unchanged Low | |
| Q9H1R3 | F22gh8 | myosin light chain kinase 2: skel | 93.58977 | 129 | 136.0456 | 119.5324 | 5.40E−01 | 4.83E−01 | 1 | Unchanged Low | |
| P28358 | P05ab6 | homeo box D10 | 108.9905 | 102 | 136.1419 | 115.6766 | 3.21E−01 | −9.71E−02 | 1 | Unchanged Low | |
| O76070 | P18cd1 | synuclein: gamma (breast cance | 128.551 | 140 | 136.1645 | 134.744 | 8.30E−02 | 1.18E−01 | 1 | Unchanged Low | |
| Q9Y5A7 | N07ef2 | NEDD8 ultimate buster-1 | 119.0653 | 195 | 136.2606 | 149.951 | 1.95E−01 | 7.08E−01 | 1 | Unchanged Low | |
| Q9NWY7 | A16gh3 | hypothetical protein FLJ20530 | 103.7638 | 110 | 136.3026 | 116.6328 | 3.94E−01 | 8.20E−02 | 1 | Unchanged Low | |
| O43715 | P02ef2 | hypothetical protein HSPC132 | 137.4694 | 110 | 136.3043 | 127.9562 | −1.23E−02 | −3.20E−01 | 1 | Unchanged Low | |
| Q9BY75 | K19gh8 | itchy homolog E3 ubiquitin protei | 118.5721 | 127 | 136.5094 | 127.3323 | 2.03E−01 | 9.81E−02 | 1 | Unchanged Low | |
| O43431 | A12ef7 | endothelial differentiation: lysoph | 126.4659 | 171 | 136.5393 | 144.6131 | 1.11E−01 | 4.34E−01 | 1 | Unchanged Low | |
| O43567 | D15cd7 | ring finger protein 13 | 110.4449 | 153 | 136.5722 | 133.3964 | 3.06E−01 | 4.72E−01 | 1 | Unchanged Low | |
| Q9Y2S7 | P13ef8 | DKFZP586F 1524 protein | 113.1366 | 104 | 136.7041 | 117.8414 | 2.73E−01 | −1.26E−01 | 1 | Unchanged Low | |
| O14582 | O24ef2 | spondyloepiphyseal dysplasia; la | 135.1666 | 135 | 136.7342 | 135.7349 | 1.66E−02 | 1.46E−03 | 1 | Unchanged Low | |
| O75056 | G12gh1 | syndecan 3 (N-syndecan) | 120.0661 | 162 | 136.797 | 139.562 | 1.88E−01 | 4.31E−01 | 1 | Unchanged Low | |
| Q9P2Y2 | D18gh4 | beta-1;4 mannosyltransferase | 89.6857 | 143 | 136.8208 | 123.2251 | 6.09E−01 | 6.75E−01 | 1 | Unchanged Low | |
| Q16595 | M08ab3 | Friedreich ataxia | 139.5857 | 130 | 136.8314 | 135.5917 | −2.85E−02 | −9.82E−02 | 1 | Unchanged Low | |
| Q14807 | B18ab7 | kinesin-like 4 | 114.4726 | 94.2 | 136.9298 | 115.2048 | 2.58E−01 | −2.81E−01 | 1 | Unchanged Low | |
| Q9BZL4 | L10gh6 | protein phosphatase 1: regulator | 110.5367 | 123 | 137.0777 | 123.5234 | 3.10E−01 | 1.54E−01 | 1 | Unchanged Low | |
| P30876 | I13ab8 | polymerase (RNA) II (DNA direct | 265.5337 | 200 | 137.1153 | 200.7168 | −9.54E−01 | −4.12E−01 | 1 | Unchanged Low | |
| Q9UGP6 | B17cd7 | coatomer protein complex: subur | 94.73601 | 134 | 137.3091 | 122.0845 | 5.35E−01 | 5.02E−01 | 1 | Unchanged Low | |
| P52655 | M01ef2 | general transcription factor IIA: 1 | 121.4896 | 128 | 137.3707 | 129.1154 | 1.77E−01 | 8.08E−02 | 1 | Unchanged Low | |
| Q9HB07 | G23gh5 | chromosome 12 open reading fra | 121.2547 | 118 | 137.4723 | 125.5248 | 1.81E−01 | −4.11E−02 | 1 | Unchanged Low | |
| Q92940 | H23ab7 | MAD: mothers against decapenta | 414.4889 | 223 | 137.6028 | 258.3032 | −1.59E+00 | −8.95E−01 | 1 | Unchanged Low | |
| Q9NWN1 | H19gh2 | hypothetical protein FLJ20727 | 117.7514 | 120 | 137.6351 | 124.9938 | 2.25E−01 | 2.24E−02 | 1 | Unchanged Low | |
| O43664 | A10cd6 | G protein-coupled receptor 66 | 108.1311 | 102 | 137.6789 | 116.0604 | 3.49E−01 | −7.90E−02 | 1 | Unchanged Low | |
| O14647 | C19ab4 | chromodomain helicase DNA bin | 96.14904 | 164 | 137.7441 | 132.5407 | 5.19E−01 | 7.68E−01 | 1 | Unchanged Low | |
| O75333 | N15cd2 | T-box 10 | 126.0065 | 129 | 137.7737 | 130.9257 | 1.29E−01 | 3.38E−02 | 1 | Unchanged Low | |
| Q16539 | J04ef5 | mitogen-activated protein kinase | 148.7339 | 219 | 137.8 | 168.4312 | −1.10E−01 | 5.57E−01 | 1 | Unchanged Low | |
| Q9NQG5 | A07gh5 | chromosome 20 open reading fra | 86.80552 | 143 | 137.803 | 122.4598 | 6.87E−01 | 7.18E−01 | 1 | Unchanged Low | |
| Q15149 | E05ab8 | plectin 1: intermediate filament b | 125.7248 | 83.9 | 138.0785 | 115.8848 | 1.35E−01 | −5.84E−01 | 1 | Unchanged Low | |
| Q9NVZ7 | G12gh3 | hypothetical protein FLJ10407 | 166.213 | 175 | 138.1444 | 159.6342 | −2.67E−01 | 7.06E−02 | 1 | Unchanged Low | |
| Q15800 | D02cd2 | sterol-C4-methyl oxidase-like | 171.8648 | 71.2 | 138.1497 | 127.0641 | −3.15E−01 | −1.27E+00 | 1 | Unchanged Low | |
| Q9H0B6 | D16gh5 | hypothetical protein FLJ12387 si | 96.54943 | 129 | 138.1542 | 121.3672 | 5.17E−01 | 4.22E−01 | 1 | Unchanged Low | |
| P09001 | K14cd1 | mitochondrial ribosomal protein | 94.17331 | 128 | 138.1601 | 120.1502 | 5.53E−01 | 4.44E−01 | 1 | Unchanged Low | |
| O00301 | D24cd3 | KH-type splicing regulatory prote | 126.0509 | 88.3 | 138.3014 | 117.5428 | 1.34E−01 | −5.14E−01 | 1 | Unchanged Low | |
| P55786 | J19ef7 | aminopeptidase puromycin sensi | 273.7328 | 98.9 | 138.3924 | 170.346 | −9.84E−01 | −1.47E+00 | 1 | Unchanged Low | |
| Q9Y5Y9 | B14cd2 | sodium channel; voltage gated; | 127.3727 | 139 | 138.4331 | 134.9928 | 1.20E−01 | 1.28E−01 | 1 | Unchanged Low | |
| P19235 | M01ef7 | erythropoietin receptor | 198.5954 | 77.3 | 138.4803 | 138.1099 | −5.20E−01 | −1.36E+00 | 1 | Unchanged Low | |
| O15228 | A16ef3 | glyceronephosphate O-acyltrans | 225.1315 | 186 | 138.5137 | 183.349 | −7.01E−01 | −2.72E−01 | 1 | Unchanged Low | |
| Q9UK97 | K05cd8 | F-box only protein 9 | 151.0501 | 104 | 138.5593 | 131.097 | −1.25E−01 | −5.43E−01 | 1 | Unchanged Low | |
| P50990 | D20cd6 | chaperonin containing TCP1: su | 274.982 | 178 | 138.6001 | 197.1593 | −9.88E−01 | −6.28E−01 | 1 | Unchanged Low | |
| Q9NZM1 | P19cd8 | fer-1-like 3: myoferlin (C. elegans | 218.8411 | 202 | 138.6209 | 186.4122 | −6.59E−01 | −1.17E−01 | 1 | Unchanged Low | |
| Q92924 | N20cd1 | SWI/SNF related: matrix associa | 131.7669 | 82.7 | 138.6764 | 117.704 | 7.37E−02 | −6.73E−01 | 1 | Unchanged Low | |
| O75845 | A18gh1 | sterol-C5-desaturase (ERG3 del | 101.9285 | 153 | 138.7232 | 131.1196 | 4.45E−01 | 5.83E−01 | 1 | Unchanged Low | |
| Q9NQ88 | I10gh4 | chromosome 12 open reading fra | 80.2401 | 158 | 138.8274 | 125.605 | 7.91E−01 | 9.75E−01 | 1 | Unchanged Low | |
| Q16515 | C19ab2 | amiloride-sensitive cation chann | 102.1159 | 109 | 138.9915 | 116.6136 | 4.45E−01 | 9.06E−02 | 1 | Unchanged Low | |
| P49759 | F16ab5 | CDC-like kinase 1 | 86.24615 | 147 | 139.0985 | 124.2553 | 6.90E−01 | 7.73E−01 | 1 | Unchanged Low | |
| P33552 | A22ab5 | CDC28 protein kinase 2 | 109.5671 | 96 | 139.1297 | 114.8881 | 3.45E−01 | −1.91E−01 | 1 | Unchanged Low | |
| O75808 | H21cd2 | small optic lobes homolog (Dros | 115.654 | 98.9 | 139.1825 | 117.8898 | 2.67E−01 | −2.26E−01 | 1 | Unchanged Low | |
| Q9NXR1 | M07gh2 | LIS1-interacting protein NUDE1; | 96.20481 | 123 | 139.1939 | 119.5057 | 5.33E−01 | 3.56E−01 | 1 | Unchanged Low | |
| O75381 | L12ab8 | peroxisomal biogenesis factor 14 | 473.8395 | 113 | 139.2209 | 241.8793 | −1.77E+00 | −2.07E+00 | 1 | Unchanged Low | |
| O60930 | P23cd1 | ribonuclease H1 | 167.3611 | 111 | 139.2393 | 139.1464 | −2.65E−01 | −5.95E−01 | 1 | Unchanged Low | |
| O14653 | P14cd4 | golgi SNAP receptor complex me | 115.7917 | 89.7 | 139.2541 | 114.9152 | 2.66E−01 | −3.68E−01 | 1 | Unchanged Low | |
| Q16540 | L24gh1 | mitochondrial ribosomal protein | 209.2433 | 120 | 139.3426 | 156.1365 | −5.87E−01 | −8.04E−01 | 1 | Unchanged Low | |
| O14658 | P06cd2 | ras-related C3 botulinum toxin s | 158.176 | 166 | 139.3634 | 154.6374 | −1.83E−01 | 7.29E−02 | 1 | Unchanged Low | |
| Q15814 | M17cd2 | tubulin-specific chaperone c | 103.3699 | 112 | 139.8311 | 118.2869 | 4.34E−01 | 1.14E−01 | 1 | Unchanged Low | |
| Q9Y3C9 | J09ef2 | CGI-127 protein | 83.4762 | 163 | 139.6599 | 128.8568 | 7.42E−01 | 9.69E−01 | 1 | Unchanged Low | |
| P18827 | F22cd1 | syndecan 1 | 114.4045 | 92.7 | 139.6607 | 115.5895 | 2.88E−01 | −3.03E−01 | 1 | Unchanged Low | |
| P04150 | B21ab3 | nuclear receptor subfamily 3: gro | 106.6921 | 103 | 139.6889 | 116.6017 | 3.89E−01 | −4.49E−02 | 1 | Unchanged Low | |
| Q9Y301 | N05ef1 | CGI-12 protein | 108.0511 | 101 | 139.7035 | 118.3594 | 3.71E−01 | −9.27E−02 | 1 | Unchanged Low | |
| O00826 | F10cd1 | small inducible cytokine subfamil | 113.478 | 106 | 139.8189 | 119.8588 | 3.01E−01 | −9.45E−02 | 1 | Unchanged Low | |
| | B18cd8 | neural precursor cell expressed; | 108.1711 | 133 | 139.9587 | 127.048 | 3.72E−01 | 2.98E−01 | 1 | Unchanged Low | |
| Q9Y311 | C01ef2 | mitochondrial ribosomal protein | 162.8519 | 74.8 | 139.9856 | 125.8788 | −2.18E−01 | −1.12E+00 | 1 | Unchanged Low | |
| Q15633 | K14cd2 | TAR (HIV) RNA binding protein 2 | 169.3779 | 125 | 140.0138 | 144.9588 | −2.75E−01 | −4.33E−01 | 1 | Unchanged Low | |
| P02278 | M22gh6 | H2B histone family: member K | 100.2794 | 216 | 140.0965 | 152.2309 | 4.82E−01 | 1.11E+00 | 1 | Unchanged Low | |
| P53672 | J11cd7 | crystallin; beta A2 | 101.7321 | 187 | 140.2804 | 142.8494 | 4.64E−01 | 8.75E−01 | 1 | Unchanged Low | |
| P09417 | C05ab8 | quinoid dihydropteridine reducta | 126.6361 | 161 | 140.3564 | 142.5395 | 1.48E−01 | 3.43E−01 | 1 | Unchanged Low | |
| Q9HAY2 | K14gh5 | MAGEF1 protein | 96.01675 | 126 | 140.3654 | 120.9341 | 5.48E−01 | 3.97E−01 | 1 | Unchanged Low | |
| P30273 | A20ab5 | Fc fragment of IgE; high affinity I | 88.48289 | 171 | 140.4541 | 133.3475 | 6.67E−01 | 9.51E−01 | 1 | Unchanged Low | |
| Q92949 | G10ab4 | forkhead box J1 | 250.4112 | 167 | 140.5084 | 185.89 | −8.34E−01 | −5.87E−01 | 1 | Unchanged Low | |
| Q9GZT9 | G01gh7 | egl nine homolog 1 (C. elegans) | 112.8025 | 99.2 | 140.6556 | 117.5626 | 3.18E−01 | −1.85E−01 | 1 | Unchanged Low | |
| P35658 | O23cd4 | nucleoporin 214kD (CAIN) | 141.8279 | 137 | 140.7761 | 139.8811 | −1.07E−02 | −4.96E−02 | 1 | Unchanged Low | |
| Q9HB40 | G01gh5 | likely homolog of rat and mouse | 105.9316 | 99.3 | 140.9548 | 115.3964 | 4.12E−01 | −9.32E−02 | 1 | Unchanged Low | |
| O14686 | O16cd3 | myeloid/lymphoid or mixed-linea | 117.844 | 157 | 141.0674 | 138.5398 | 2.60E−01 | 4.11E−01 | 1 | Unchanged Low | |
| Q92911 | O12cd1 | solute carrier family 5 (sodium io | 264.1256 | 125 | 141.0904 | 176.594 | −9.05E−01 | −1.08E+00 | 1 | Unchanged Low | |
| Q9Y5E4 | B18ef3 | protocadherin beta 5 | 95.08061 | 148 | 141.1894 | 127.9379 | 5.70E−01 | 6.34E−01 | 1 | Unchanged Low | |
| Q13057 | H17gh7 | nucleotide binding protein | 186.8796 | 125 | 141.2202 | 151.0906 | −4.04E−01 | −5.78E−01 | 1 | Unchanged Low | |
| Q14863 | B23ab8 | POU domain; class 6; transcripti | 103.2077 | 103 | 141.3258 | 115.7954 | 4.53E−01 | −4.97E−03 | 1 | Unchanged Low | |
| P36610 | G10ef3 | frequenin homolog (Drosophila) | 110.2191 | 136 | 141.4925 | 129.0874 | 3.60E−01 | 2.98E−01 | 1 | Unchanged Low | |
| O60240 | O12ab8 | perilipin | 1121.108 | 52.2 | 141.5046 | 438.2653 | −2.99E+00 | −4.43E+00 | 1 | Unchanged Low | |
| Q9H9B4 | P05gh5 | hypothetical protein FLJ12876 | 87.99841 | 133 | 141.7125 | 120.8973 | 6.87E−01 | 5.96E−01 | 1 | Unchanged Low | |
| O00584 | J14cd3 | ribonuclease 6 precursor | 124.6932 | 250 | 141.8008 | 172.0542 | 1.85E−01 | 1.00E+00 | 1 | Unchanged Low | |
| Q9H658 | M04gh7 | G protein-coupled receptor 107 | 158.2595 | 214 | 141.8139 | 171.1973 | −1.58E−01 | 4.32E−01 | 1 | Unchanged Low | |
| P18858 | K14ef6 | ligase I: DNA: ATP-dependent | 92.33071 | 128 | 141.8761 | 120.8853 | 6.20E−01 | 4.76E−01 | 1 | Unchanged Low | |
| Q02877 | D23cd1 | ribosomal protein L26 | 449.1728 | 297 | 141.8851 | 295.8854 | −1.66E+00 | −5.99E−01 | 1 | Unchanged Low | |
| P15586 | J07ab4 | glucosamine (N-acetyl)-6-sulfata | 261.466 | 119 | 141.968 | 174.2861 | −8.81E−01 | −1.13E+00 | 1 | Unchanged Low | |
| P42696 | H08ef4 | KIAA0117 protein | 110.9202 | 231 | 141.9948 | 161.4649 | 3.56E−01 | 1.06E+00 | 1 | Unchanged Low | |
| O75710 | A17ef1 | molybdenum cofactor synthesis | 105.3087 | 101 | 142.0352 | 116.2449 | 4.32E−01 | −5.47E−02 | 1 | Unchanged Low | |
| Q9NP18 | C01gh2 | Fanconi anemia; complemantati | 193.8104 | 214 | 142.0643 | 183.3737 | −4.48E−01 | 1.45E−01 | 1 | Unchanged Low | |
| Q9HAR6 | O10gh5 | uterine-derived 14kDa protein | 118.9551 | 198 | 142.0669 | 152.8734 | 2.56E−01 | 7.32E−01 | 1 | Unchanged Low | |
| P15018 | O18ef7 | leukemia inhibitory factor (cholin | 253.8099 | 309 | 142.2733 | 235.0602 | −8.35E−01 | 2.84E−01 | 1 | Unchanged Low | |
| Q13546 | A23cd4 | receptor (TNFRSF)-interacting s | 139.7074 | 131 | 142.2746 | 137.7012 | 2.63E−02 | −9.15E−02 | 1 | Unchanged Low | |
| P24298 | H01ab4 | glutamic-pyruvate transaminase | 210.426 | 97.1 | 142.3123 | 149.9584 | −5.64E−01 | −1.12E+00 | 1 | Unchanged Low | |
| Q14129 | C22cd4 | DiGeorge syndrome critical regio | 112.1253 | 141 | 142.4302 | 131.8973 | 3.45E−01 | 3.32E−01 | 1 | Unchanged Low | |
| Q9BQA2 | K09gh8 | KIAA1882 protein | 164.9357 | 187 | 142.4325 | 164.7437 | −2.12E−01 | 1.80E−01 | 1 | Unchanged Low | |
| Q9UJX9 | B12ef3 | hypothetical protein; estradiol-in | 121.7396 | 98.5 | 142.5053 | 120.9085 | 2.27E−01 | −3.06E−01 | 1 | Unchanged Low | |
| Q99590 | I01cd5 | splicing factor; arginine/serine-ri | 231.0328 | 199 | 142.5273 | 190.7413 | −6.97E−01 | −2.18E−01 | 1 | Unchanged Low | |
| O14798 | C06ef7 | tumor necrosis factor receptor su | 142.6951 | 213 | 142.5294 | 166.073 | −1.68E−03 | 5.78E−01 | 1 | Unchanged Low | |
| P32238 | P01ab3 | cholecystokinin A receptor | 487.368 | 168 | 142.5911 | 265.9537 | −1.77E+00 | −1.54E+00 | 1 | Unchanged Low | |
| Q9UQ90 | C17cd2 | spastic paraplegia 7: paraplegin | 101.2857 | 146 | 142.6321 | 129.9052 | 4.94E−01 | 5.26E−01 | 1 | Unchanged Low | |
| P82980 | M03gh8 | retinol binding protein 5; cellular | 111.2191 | 93.9 | 142.6923 | 115.9454 | 3.60E−01 | −2.44E−01 | 1 | Unchanged Low | |
| P49716 | P15ab5 | CCAAT/enhancer binding protei | 169.1889 | 73.4 | 142.8277 | 128.4871 | −2.44E−01 | −1.20E+00 | 1 | Unchanged Low | |
| P20062 | M12cd1 | transcobalamin II; macrocytic an | 125.6347 | 141 | 142.8402 | 136.6279 | 1.85E−01 | 1.71E−01 | 1 | Unchanged Low | |
| P21580 | B06cd2 | tumor necrosis factor; alpha-indu | 275.8225 | 317 | 142.8664 | 245.0939 | −9.49E−01 | 1.99E−01 | 1 | Unchanged Low | |
| Q99774 | H01cd5 | protein phosphatase 4; regulator | 195.85 | 173 | 143.1867 | 170.7835 | −4.52E−01 | −1.76E−01 | 1 | Unchanged Low | |
| Q9P0T1 | A17ef3 | hypothetical protein HSPC192 | 104.4363 | 101 | 143.2517 | 116.1881 | 4.56E−01 | −5.00E−02 | 1 | Unchanged Low | |
| P06737 | B18ab8 | phosphorylase; glycogen; liver ( | 85.14818 | 139 | 143.2732 | 122.6343 | 7.51E−01 | 7.12E−01 | 1 | Unchanged Low | |
| O15063 | K10gh1 | KIAA0355 gene product | 149.9191 | 144 | 143.2945 | 145.705 | −6.52E+02 | −5.91E−02 | 1 | Unchanged Low | |
| P02743 | K14ab2 | amyloid P component; serum | 108.0152 | 94.7 | 143.3031 | 115.3343 | 4.08E−01 | −1.90E−01 | 1 | Unchanged Low | |
| Q9NVT0 | I14gh3 | hypothetical protein FLJ10534 | 136.4264 | 109 | 143.3056 | 129.6057 | 7.10E−02 | −3.23E−01 | 1 | Unchanged Low | |
| Q9Y4P3 | O07ef3 | transducin (beta)-like 2 | 112.0852 | 104 | 143.4853 | 119.7013 | 3.56E−01 | −1.14E−01 | 1 | Unchanged Low | |
| Q13886 | N14ab3 | basic transcription element bindi | 164.4049 | 88.7 | 143.5269 | 132.2194 | −1.96E−01 | −8.90E−01 | 1 | Unchanged Low | |
| Q9NVP8 | L19gh4 | hypothetical protein FLJ10595 | 101.7956 | 106 | 143.5829 | 117.1232 | 4.96E−01 | 5.83E−02 | 1 | Unchanged Low | |
| P08240 | G05cd2 | signal recognition particle recept | 158.3565 | 132 | 143.6823 | 144.5673 | −1.40E−01 | −2.66E−01 | 1 | Unchanged Low | |
| P49459 | M19ef5 | ubiquitin-conjugating enzyme E2 | 98.6462 | 170 | 143.74 | 137.6267 | 5.43E−01 | 7.89E−01 | 1 | Unchanged Low | |
| Q9NVA8 | L16gh2 | solute carrier family 38; member | 112.6488 | 127 | 143.7595 | 127.9498 | 3.52E−01 | 1.78E−01 | 1 | Unchanged Low | |
| P25325 | K23cd3 | mercaptopyruvate sulfurtransfera | 288.1435 | 274 | 143.8077 | 235.1972 | −1.00E+00 | −7.45E−02 | 1 | Unchanged Low | |
| Q9UH16 | K08cd7 | DEAD/H (Asp-Glu-Ala-Asp/His) | 135.6699 | 209 | 143.8383 | 162.7208 | 8.43E−02 | 6.21E−01 | 1 | Unchanged Low | |
| P52298 | H01cd7 | nuclear cap binding protein subu | 138.9223 | 191 | 143.6819 | 157.996 | 5.06E−02 | 4.61E−01 | 1 | Unchanged Low | |
| Q9NQ30 | A10cd7 | endothelial cell-specific molecule | 104.1846 | 122 | 143.9703 | 123.2921 | 4.67E−01 | 2.24E−01 | 1 | Unchanged Low | |
| O00541 | I08ef3 | pescadillo hOmolog 1; containing | 180.43 | 204 | 144.0988 | 176.2086 | −3.24E−01 | 1.78E−01 | 1 | Unchanged Low | |
| Q9H2V9 | B06gh7 | hypothetical protein CDA08 | 173.5608 | 169 | 144.1057 | 162.3327 | −2.68E−01 | −3.56E−02 | 1 | Unchanged Low | |
| Q9NNX1 | F12gh1 | luftelin 1 | 147.0323 | 175 | 144.106 | 155.2699 | −2.90E−02 | 2.49E−01 | 1 | Unchanged Low | |
| Q13508 | H14ab2 | ADP-ribosyltransferase 3 | 109.4052 | 101 | 144.1545 | 118.095 | 3.98E−01 | −1.19E−01 | 1 | Unchanged Low | |
| Q9NR30 | B21gh6 | DEAD/H (Asp-Glu-Ala-Asp/His) | 110.4836 | 130 | 144.2348 | 128.2929 | 3.85E−01 | 2.36E−01 | 1 | Unchanged Low | |
| Q14320 | E15cd5 | DNA segment on chromosome X | 102.5057 | 178 | 144.2677 | 141.6929 | 4.93E−01 | 7.99E−01 | 1 | Unchanged Low | |
| Q04726 | D03cd2 | transducin-like enhancer of split | 211.8773 | 115 | 144.3217 | 156.9544 | −5.54E−01 | −8.86E−01 | 1 | Unchanged Low | |
| P20061 | N13cd1 | transcobalamin I (vitamin B12 bi | 117.3445 | 85.5 | 144.4835 | 115.787 | 3.00E−01 | −4.56E−01 | 1 | Unchanged Low | |
| Q9Y2Y1 | J12ef2 | polymerase (RNA) III (DNA direc | 153.7426 | 145 | 144.5561 | 147.7222 | −8.89E−02 | −8.58E−02 | 1 | Unchanged Low | |
| P35754 | G22ab5 | glutaredoxin (thioltransfarase) | 153.823 | 138 | 144.7308 | 145.4287 | −8.79E−02 | −1.59E−01 | 1 | Unchanged Low | |
| Q02978 | F01cd3 | solute carrier family 25 (mitochor | 106.877 | 98.2 | 144.7822 | 116.628 | 4.38E−01 | −1.22E−01 | 1 | Unchanged Low | |
| O95792 | N20gh2 | protein associated with PRK1 | 130.6914 | 146 | 144.7909 | 140.5872 | 1.48E−01 | 1.63E−01 | 1 | Unchanged Low | |
| Q13232 | D20ef7 | non-metastatic cells 3; protein ex | 90.7906 | 156 | 144.8861 | 130.592 | 6.74E−01 | 7.82E−01 | 1 | Unchanged Low | |
| Q9NVH2 | L01ef8 | DKFZP434B168 protein | 117.6587 | 91.2 | 144.9821 | 117.932 | 3.01E−01 | −3.68E−01 | 1 | Unchanged Low | |
| Q15561 | O15cd2 | TEA domain family mamber 4 | 234.306 | 172 | 145.3307 | 183.8604 | −6.89E−01 | −4.46E−01 | 1 | Unchanged Low | |
| Q9UPN9 | B01ef2 | tripartite motif-containing 33 | 110.9269 | 135 | 145.3321 | 130.2668 | 3.90E−01 | 2.78E−01 | 1 | Unchanged Low | |
| Q14353 | O06ab3 | guanidinoacetate N-methyltransf | 109.1385 | 103 | 145.3798 | 119.1987 | 4.14E−01 | −8.24E−02 | 1 | Unchanged Low | |
| Q9NZM5 | A18ef4 | glioma tumor suppressor candida | 150.098 | 122 | 145.587 | 139.1802 | −4.406−02 | −3.01E−01 | 1 | Unchanged Low | |
| O43255 | B11cd2 | seven in absentia homolog 2 (Dr | 114.6318 | 101 | 145.653 | 120.4593 | 3.46E−01 | −1.81E−01 | 1 | Unchanged Low | |
| Q14331 | G04ab5 | FSHD region gene 1 | 164.0964 | 176 | 145.6576 | 161.7807 | −1.72E−01 | 9.77E−02 | 1 | Unchanged Low | |
| P18847 | N23ab5 | activating transcription factor 3 | 127.5442 | 154 | 145.6654 | 142.5299 | 1.92E−01 | 2.75E−01 | 1 | Unchanged Low | |
| O60258 | I07cd4 | fibroblast growth factor 17 | 159.7671 | 223 | 145.6821 | 176.0472 | −1.33E−01 | 4.79E−01 | 1 | Unchanged Low | |
| P47929 | P02ab6 | lectin; galactoside-binding; solub | 151.1595 | 92.8 | 145.699 | 129.8767 | −5.31E−02 | −7.04E−01 | 1 | Unchanged Low | |
| P35227 | O14cd4 | zinc finger protein 144 (Mel-18) | 108.2295 | 151 | 145.8554 | 135.0089 | 4.30E−01 | 4.80E−01 | 1 | Unchanged Low | |
| Q15599 | O15cd5 | solute carrier family 9 (sodium/hy | 168.9673 | 80.5 | 145.993 | 131.8082 | −2.11E−01 | −1.07E+00 | 1 | Unchanged Low | |
| Q9UKJ5 | B20cd7 | cystein-rich hydrophobic domain | 164.4717 | 208 | 145.9967 | 178.8637 | −3.37E−01 | 1.60E−01 | 1 | Unchanged Low | |
| Q9HCB9 | C10gh5 | chromosome 1 open reading fra | 94.16759 | 126 | 146.1683 | 122.1779 | 8.34E−01 | 4.22E−01 | 1 | Unchangnd Low | |
| Q9Y3A0 | K07ef2 | CGI-92 protein | 119.1033 | 176 | 146.1698 | 147.7783 | 2.95E−01 | 5.80E−01 | 1 | Unchanged Low | |
| P49815 | M17ef6 | tuberous sclerosis 2 | 106.0159 | 163 | 146.2712 | 138.4231 | 4.64E−01 | 6.20E−01 | 1 | Unchanged Low | |
| P25116 | L19ab4 | coagulation factor II (thrombin) re | 82.48228 | 143 | 146.2742 | 123.8585 | 8.27E−01 | 7.92E−01 | 1 | Unchanged Low | |
| O75829 | O01cd7 | chondromodulin I precursor | 121.7658 | 119 | 146.4876 | 128.9495 | 2.67E−01 | −3.81E−02 | 1 | Unchanged Low | |
| Q9NYI1 | M16ef2 | ECSIT | 111.7926 | 110 | 146.6345 | 122.8588 | 3.91E−01 | −2.14E−02 | 1 | Unchanged Low | |
| P24347 | I24ef5 | matrix metalloproteinase 11 (stro | 97.57916 | 136 | 146.649 | 126.9018 | 5.88E−01 | 4.84E−01 | 1 | Unchanged Low | |
| O43189 | K24ab8 | PHD finger protein 1 | 112.1101 | 101 | 146.6925 | 120.038 | 3.88E−01 | −1.46E−01 | 1 | Unchanged Low | |
| Q9P029 | N22ef2 | TH1-like (Drosophila) | 201.2292 | 159 | 146.6998 | 168.9188 | −4.56E−01 | −3.41E−01 | 1 | Unchanged Low | |
| Q9NZP9 | E17ef2 | immediate early response 5 | 105.1683 | 157 | 146.761 | 136.3041 | 4.81E−01 | 5.78E−01 | 1 | Unchanged Low | |
| Q9UIA3 | I12ef2 | neutral sphingomyelinase | 165.6408 | 147 | 146.8134 | 153.0756 | −1.74E−01 | −1.74E−01 | 1 | Unchanged Low | |
| Q99734 | M12ef5 | Notch homotog 2 (Drosophila) | 220.4251 | 263 | 147.0111 | 210.2011 | −5.84E−01 | 2.56E−01 | 1 | Unchanged Low | |
| O00411 | C19cd1 | polymerase (RNA) mitochondrial | 120.5078 | 105 | 147.0481 | 124.1084 | 2.87E−01 | −2.02E−01 | 1 | Unchanged Low | |
| P28347 | P04gh1 | TEA domain family member 1 (S | 230.2101 | 174 | 147.0913 | 183.776 | −6.46E−01 | −4.04E−01 | 1 | Unchanged Low | |
| Q13619 | G19gh1 | cullin 4A | 153.1592 | 218 | 147.2043 | 172.0621 | −5.72E−02 | 5.07E−01 | 1 | Unchanged Low | |
| O94929 | O06ef8 | KIAA0843 protein | 108.7821 | 99.4 | 147.2921 | 118.4779 | 4.37E−01 | −1.31E−01 | 1 | Unchanged Low | |
| Q9P026 | A02ef8 | HSPC134 protein | 115.4895 | 184 | 147.3205 | 149.0331 | 3.51E−01 | 6.74E−01 | 1 | Unchanged Low | |
| P28067 | D08ab5 | major histocompatibility complex; | 128.7098 | 100 | 147.3934 | 125.4983 | 1.96E−01 | −3.58E−01 | 1 | Unchanged Low | |
| Q14126 | E11ef7 | desmoglein 2 | 426.8387 | 366 | 147.4196 | 313.3393 | −1.53E+00 | −2.23E−01 | 1 | Unchanged Low | |
| Q14267 | P01cd1 | regulatory factor X; 4 (influences | 127.7912 | 98.9 | 147.4351 | 124.7021 | 2.06E−01 | −3.70E−01 | 1 | Unchanged Low | |
| P55197 | H09ab2 | myeloid/lymphoid or mixed-lineag | 160.668 | 185 | 147.4439 | 164.3164 | −1.24E−01 | 2.02E−01 | 1 | Unchanged Low | |
| Q9NP87 | O20cd8 | polymerase (DNA directed); mu | 115.1765 | 86.7 | 147.4845 | 116.4497 | 3.57E−01 | −4.10E−01 | 1 | Unchanged Low | |
| P04155 | A04cd2 | trefoil factor 1 (breast cancer: es | 258.058 | 136 | 147.487 | 180.4791 | −8.07E−01 | −9.25E−01 | 1 | Unchanged Low | |
| Q9C069 | N02gh7 | sec13-like protein | 52.65015 | 424 | 147.5255 | 207.9658 | 1.49E+00 | 3.01E+00 | 1 | Unchanged Low | |
| P12270 | G14cd2 | translocated promoter region (to | 104.4493 | 136 | 147.7447 | 129.4065 | 5.00E−01 | 3.81E−01 | 1 | Unchanged Low | |
| Q9UIG6 | Il4ef2 | LPAP for lysophosphatidic acid p | 218.3106 | 325 | 147.8779 | 230.2685 | −5.62E−01 | 5.72E−01 | 1 | Unchanged Low | |
| O15453 | P08cd5 | NBR2 | 99.08545 | 108 | 147.9143 | 118.2765 | 5.78E−01 | 1.22E−01 | 1 | Unchanged Low | |
| Q9UNW8 | F17cd8 | G protein-coupled receptor | 239.3255 | 347 | 148.021 | 244.7569 | −6.93E−01 | 5.36E−01 | 1 | Unchanged Low | |
| P06746 | I10ef6 | polymerase (DNA directed); beta | 142.5013 | 225 | 148.0351 | 171.8166 | 5.50E−02 | 6.58E−01 | 1 | Unchanged Low | |
| O15414 | I08cd7 | trinucleotide repeat containing 4 | 131.6219 | 199 | 148.0947 | 159.7305 | 1.70E−01 | 6.00E−01 | 1 | Unchanged Low | |
| O15259 | L22ab7 | nephronophthisis 1 (juvenile) | 115.0436 | 110 | 148.3613 | 124.437 | 3.67E−01 | −6.59E−02 | 1 | Unchanged Low | |
| Q9NRA8 | P05gh4 | eIF4E-transporter | 106.343 | 178 | 148.4323 | 144.3142 | 4.81E−01 | 7.45E−01 | 1 | Unchanged Low | |
| Q9NP77 | C10ef8 | HSPC182 protein | 171.17 | 175 | 148.5234 | 165.0839 | −2.05E−01 | 3.60E−02 | 1 | Unchanged Low | |
| Q06587 | P17cd1 | ring finger protein 1 | 119.2377 | 127 | 148.657 | 131.6381 | 3.18E−01 | 9.12E−02 | 1 | Unchanged Low | |
| Q9Y5Z5 | E22ef2 | heme binding protein 1 | 181.0768 | 196 | 148.7681 | 175.2357 | −2.84E−01 | 1.13E−01 | 1 | Unchanged Low | |
| O15327 | I05cd4 | inositol polyphosphate-4-phosph | 183.6815 | 131 | 148.785 | 154.5722 | −3.04E−01 | −4.85E−01 | 1 | Unchanged Low | |
| P49646 | P19cd1 | regulator of mitotic spindle assen | 143.9463 | 117 | 148.8487 | 136.7074 | 4.83E−02 | −2.95E−01 | 1 | Unchanged Low | |
| Q99809 | F16cd5 | conserved gene amplified in oste | 134.0126 | 91.3 | 148.9901 | 124.7728 | 1.53E−01 | −5.53E−01 | 1 | Unchanged Low | |
| Q9UBM1 | G02cd7 | phosphatidylelhanolamine N-mel | 138.4849 | 122 | 149.0109 | 136.3565 | 1.06E−01 | −1.88E−01 | 1 | Unchanged Low | |
| P16403 | G04ab6 | H1 histone family; member 2 | 427.1258 | 558 | 149.1016 | 377.9287 | −1.52E+00 | 3.84E−01 | 1 | Unchanged Low | |
| O75503 | B01ab4 | ceroid-lipofuscinosis; neuronal 5 | 148.9991 | 75.4 | 149.1361 | 124.5021 | 1.33E−03 | −9.83E−01 | 1 | Unchanged Low | |
| P15814 | P20gh6 | immunoglobulin lambda-like poly | 75.91644 | 144 | 149.2005 | 123.9169 | 9.19E−01 | 8.64E−01 | 1 | Unchanged Low | |
| Q03164 | N01ab7 | myeloid/lymphoid or mixed-lineag | 157.0713 | 81.9 | 149.358 | 129.4559 | −7.26E−02 | −9.39E−01 | 1 | Unchanged Low | |
| Q13624 | B08ef6 | interleukin enhancer binding fact | 136.4070 | 130 | 149.4265 | 138.6386 | 1.32E−01 | −6.85E−02 | 1 | Unchanged Low | |
| Q14118 | M02ab5 | dystroglycan 1 (dystrophin-asso | 105.7987 | 99 | 149.6192 | 118.1383 | 5.00E−01 | −9.59E−02 | 1 | Unchanged Low | |
| Q13286 | E20ab3 | ceroid-lipofuscinosis; neuronal 3: | 240.521 | 302 | 149.6542 | 230.6187 | −6.85E−01 | 3.27E−01 | 1 | Unchanged Low | |
| Q9Y5Q0 | E15gh7 | fatty acid desaturase 3 | 117.0825 | 115 | 149.704 | 127.2281 | 3.55E−01 | −2.72E−02 | 1 | Unchanged Low | |
| Q9P0R1 | K08ef2 | forkhead box P1 | 99.17408 | 114 | 149.7517 | 120.8481 | 5.95E−01 | 1.96E−01 | 1 | Unchanged Low | 1 Unchanged Low |
| P41182 | C24ab5 | B-cell CLL/lymphoma 6 (zinc fing | 115.9704 | 85.9 | 149.853 | 117.2275 | 3.70E−01 | −4.34E−01 | 1 | Unchanged Low | |
| O75497 | K14cd6 | microspherule protein 1 | 145.7927 | 163 | 149.9313 | 153.0143 | 4.04E−02 | 1.64E−01 | 1 | Unchanged Low | |
| Q14185 | M19ab4 | dedicator of cyto-kinesis 1 | 95.3322 | 112 | 150.0398 | 118.9819 | 6.54E−01 | 2.27E−01 | 1 | Unchanged Low | |
| O95163 | P05cd3 | inhibitor of kappa tight polypeptic | 203.0161 | 120 | 150.0489 | 157.6357 | −4.36E−01 | −7.60E−01 | 1 | Unchanged Low | |
| Q14508 | L05ef7 | WAP four-disulfide core domain; | 102.6084 | 201 | 150.0782 | 151.3745 | 5.49E−01 | 9.73E−01 | 1 | Unchanged Low | |
| P25942 | M21ef7 | tumor necrosis factor receptor su | 110.0933 | 239 | 150.083 | 166.2403 | 4.47E−01 | 1.12E+00 | 1 | Unchanged Low | |
| Q9Y6E0 | F15cd3 | serine/threonine kinase 24 (STE: | 206.7079 | 225 | 150.279 | 194.1145 | −4.60E−01 | 1.25E−01 | 1 | Unchanged Low | |
| Q9UQF2 | J21cd5 | mitogen-acfivaled protein kinase | 229.9384 | 182 | 150.2902 | 187.4786 | −6.13E−01 | −3.36E−01 | 1 | Unchanged Low | |
| Q9Y285 | D11ab5 | phenylalanine-tRNA synthetase-I | 179.7613 | 145 | 150.3716 | 158.2291 | −2.58E−01 | −3.14E−01 | 1 | Unchanged Low | |
| Q04762 | F09ab4 | cell matrix adhesion regulator | 134.1594 | 176 | 150.3997 | 153.4581 | 1.65E−01 | 3.90E−01 | 1 | Unchanged Low | |
| Q9Y6J9 | P19ef6 | TAF6-like RNA polymerase II; p3 | 77.59128 | 158 | 150.4181 | 128.6968 | 9.55E−01 | 1.03E+00 | 1 | Unchanged Low | |
| O43913 | C06ab8 | origin recognition complex: subu | 147.9187 | 168 | 150.4866 | 155.6272 | 2.48E−02 | 1.88E−01 | 1 | Unchanged Low | |
| P25208 | M05cd1 | nuclear transcription factor Y; be | 106.4862 | 138 | 150.5231 | 131.7807 | 4.99E−01 | 3.77E−01 | 1 | Unchanged Low | |
| Q9H9X4 | F03gh5 | hypothetical protein FLJI1618 | 189.58 | 179 | 150.5879 | 173.1977 | −3.32E−01 | −7.94E−02 | 1 | Unchanged Low | |
| O95297 | P17cd4 | myelin protein zero-like 1 | 127.461 | 121 | 150.7509 | 133.0095 | 2.42E−01 | −7.72E−02 | 1 | Unchanged Low | |
| Q9UPG8 | O06ab8 | pleiomorphic adenoma gene-like | 134.8747 | 165 | 150.9533 | 150.4388 | 1.62E−01 | 2.95E−01 | 1 | Unchanged Low | |
| P16333 | H16ef5 | NCK adaptor protein 1 | 237.1463 | 351 | 150.9549 | 246.4108 | −6.52E−01 | 5.66E−01 | 1 | Unchanged Low | |
| Q14108 | I22ab5 | CD36 antigen (collagen type I re | 155.206 | 139 | 150.9785 | 148.3416 | −3.98E−02 | −1.61E−01 | 1 | Unchanged Low | |
| Q9UFF9 | O05cd5 | CCR4-NOT transcription comple: | 162.9025 | 208 | 151.0687 | 174.1041 | −1.09E−01 | 3.55E−01 | 1 | Unchanged Low | |
| O15242 | O19ab8 | nardilysin (N-arginine dibasic co | 210.0184 | 198 | 151.1245 | 186.2788 | −4.75E−01 | −8.72E−02 | 1 | Unchanged Low | |
| Q13442 | N15ef3 | PDGFA associated protein 1 | 156.1376 | 188 | 151.1314 | 165.0545 | −4.70E−02 | 2.87E−01 | 1 | Unchanged Low | |
| Q99755 | A15ef6 | phosphatidylinositol-4-phosphate | 112.2177 | 146 | 151.3432 | 138.4804 | 4.32E−01 | 3.78E−01 | 1 | Unchanged Low | |
| Q9NZV1 | E09gh2 | cysteine-rich motor neuron 1 | 112.5932 | 99.1 | 151.3541 | 121.0073 | 4.27E−01 | −1.85E−01 | 1 | Unchanged Low | |
| Q05195 | C09ab7 | MAX dimerization protein | 136.8996 | 254 | 151.5662 | 180.6838 | 1.47E−01 | 8.89E−01 | 1 | Unchanged Low | |
| O95671 | J14ab2 | acetylserotonin O-methyltransfer | 99.67178 | 98.5 | 151.7084 | 116.6189 | 6.06E−01 | −1.74E−02 | 1 | Unchanged Low | |
| Q9Y658 | A09ef2 | RNA helicase | 112.9715 | 95.3 | 151.8988 | 120.0494 | 4.27E−01 | −2.46E−01 | 1 | Unchanged Low | |
| Q9BTU6 | N12gh6 | phosphatidylinositol 4-kinase typ | 97.30862 | 178 | 151.965 | 142.5387 | 8.43E−01 | 8.74E−01 | 1 | Unchanged Low | |
| Q9UPN6 | G04ef8 | KIAA1116 protein | 127.681 | 113 | 152.0268 | 130.9048 | 2.52E−01 | −1.76E−01 | 1 | Unchanged Low | |
| O00170 | J03ab2 | aryl hydrocarbon receptor intera | 555.1127 | 223 | 152.0508 | 310.1793 | −1.87E+00 | −1.31E+00 | 1 | Unchanged Low | |
| Q9UJW6 | P01ef3 | activity-regulated cytoskeleton-a | 221.8559 | 185 | 152.1474 | 186.4423 | −5.44E−01 | −2.60E−01 | 1 | Unchanged Low | |
| Q9HCT0 | N02ef8 | fibroblast growth factor 22 | 157.7471 | 126 | 152.2042 | 145.1536 | −5.16E−02 | −3.30E−01 | 1 | Unchanged Low | |
| Q15459 | O03cd6 | splicing factor 3a; subunit 1:120 | 163.4432 | 145 | 152.3945 | 153.5048 | −1.01E−01 | −1.76E−01 | 1 | Unchanged Low | |
| O95400 | G16cd6 | CD2 antigen (cytoplasmic tail) bi | 192.2313 | 238 | 152.5257 | 194.1745 | −3.34E−01 | 3.07E−01 | 1 | Unchanged Low | |
| P07954 | M06ab3 | fumerate hydratase | 121.2546 | 142 | 152.5652 | 138.7259 | 3.31E−01 | 2.31E−01 | 1 | Unchanged Low | |
| Q9H6Z5 | C16gh5 | hypothetical protein FLJ21634 | 101.1467 | 152 | 152.6745 | 135.2933 | 5.94E−01 | 5.88E−01 | 1 | Unchanged Low | |
| Q9HCN8 | A05gh1 | stromal cell-derived factor 2-like | 136.5604 | 128 | 152.9097 | 139.1775 | 1.63E−01 | −9.27E−02 | 1 | Unchanged Low | |
| Q92890 | A24cd4 | ubiquitin fusion degradation 1-lik | 157.2559 | 220 | 152.9538 | 176.6159 | −4.00E−02 | 4.82E−01 | 1 | Unchanged Low | |
| O43324 | N24cd4 | eukaryotic translation elongation | 150.6748 | 112 | 152.9613 | 138.6164 | 2.17E−02 | −4.25E−01 | 1 | Unchanged Low | |
| Q9NVU8 | H10gh2 | hypothetical protein FLJ10496 | 145.4398 | 119 | 152.9651 | 139.1241 | 7.28E−02 | −2.90E−01 | 1 | Unchanged Low | |
| Q9NQW6 | H01ef4 | anillin; actin binding protein (scra | 113.8655 | 85.6 | 152.992 | 117.4953 | 4.26E−01 | −4.11E−01 | 1 | Unchanged Low | |
| Q9NWH4 | L01gh2 | mitochondrial ribosomal protein | 173.4347 | 107 | 153.0783 | 144.3461 | −1.80E−01 | −7.03E−01 | 1 | Unchanged Low | |
| Q9UBV8 | M13cd8 | PEF protein with a long N-termin | 148.0371 | 144 | 153.0828 | 148.3188 | 4.84E−02 | −4.156−02 | 1 | Unchanged Low | |
| P15328 | F10ab3 | folate receptor 1 (adult) | 152.5051 | 123 | 153.1183 | 142.7655 | 5.77E−03 | −3.14E−01 | 1 | Unchanged Low | |
| Q9Y583 | B01cd6 | NS1-associated protein 1 | 143.6283 | 177 | 153.2797 | 157.8847 | 9.38E−02 | 2.99E−01 | 1 | Unchanged Low | |
| Q07326 | M08ab8 | phosphatidylinositol glycan; clas | 132.6521 | 93 | 153.3084 | 126.3257 | 2.09E−01 | −5.12E−01 | 1 | Unchanged Low | |
| O75396 | M12cd5 | SEC22 vesicle trafficking protein | 143.9018 | 153 | 153.3626 | 150.1024 | 9.19E−02 | 8.886−02 | 1 | Unchanged Low | |
| Q9Y2Z8 | D09ef2 | CGI-09 protein | 117.2943 | 110 | 153.5282 | 126.8764 | 3.88E−01 | −9.526−02 | 1 | Unchanged Low | |
| Q9H3P7 | N05gh5 | golgi phosphoprotein 1 | 251.3681 | 258 | 153.65 | 220.9899 | −7.10E−01 | 3.736−02 | 1 | Unchanged Low | |
| O95678 | E03cd5 | cytokeratin type II | 339.3692 | 195 | 153.797 | 229.3684 | −1.14E+00 | −8.00E−01 | 1 | Unchanged Low | |
| P17026 | M04cd4 | zinc finger protein 22 (KOX 15) | 152.726 | 88.3 | 153.8779 | 131.6407 | 1.08E−02 | −7.90E−01 | 1 | Unchanged Low | |
| Q9Y3N9 | M05gh7 | olfactory receptor: family 2: subfa | 127.2527 | 137 | 153.9228 | 139.4653 | 2.75E−01 | 1.09E−01 | 1 | Unchanged Low | |
| Q9BYT8 | K08gh7 | neurolysin (metallopeptidase M3 | 85.99781 | 193 | 154.0005 | 144.2719 | 8.41E−01 | 1.16E+00 | 1 | Unchanged Low | |
| Q9UHQ7 | F02ef8 | pp21 homolog | 119.2248 | 136 | 154.0057 | 136.5651 | 3.69E−01 | 1.95E−01 | 1 | Unchanged Low | |
| O60921 | A22ab7 | HUS1 checkpoint homolog (S. po | 148.5718 | 204 | 154.126 | 169.0172 | 5.29E−02 | 4.60E−01 | 1 | Unchanged Low | |
| Q00400 | D05ab2 | acetyl-Coenzyme A transporter | 196.9505 | 124 | 154.2012 | 158.3654 | −3.53E−01 | −6.68E−01 | 1 | Unchanged Low | |
| Q9Y690 | H01gh6 | mortality factor 4 | 253.2426 | 259 | 154.2307 | 222.1522 | −7.15E−01 | 3.23E−02 | 1 | Unchanged Low | |
| Q9NZL9 | L24ef7 | methionine adenosyltransferase | 128.4144 | 99.7 | 154.2681 | 127.4733 | 2.65E−01 | −3.65E−01 | 1 | Unchanged Low | |
| O60563 | A05ab4 | cyclin T1 | 179.5706 | 171 | 154.2911 | 168.3854 | −2.19E−01 | −6.816−02 | 1 | Unchanged Low | |
| P20591 | M03ab7 | myxovirus (influenza virus) resist | 459.0611 | 873 | 154.338 | 495.4752 | −1.57E+00 | 9.27E−01 | 1 | Unchanged Low | |
| Q9BQC6 | P14gh5 | mitochondrial ribosomal protein | 188.6422 | 74.3 | 154.357 | 139.1028 | −2.89E−01 | −1.34E+00 | 1 | Unchanged Low | |
| P28838 | J02ef1 | leucine aminopeptidase 3 | 409.1007 | 383 | 154.3828 | 315.6553 | −1.41E+00 | −9.336−02 | 1 | Unchanged Low | |
| Q9NZE5 | P08ef1 | hypothetical protein LOC51319 | 135.1502 | 171 | 154.3904 | 153.3881 | 1.92E−01 | 3.36E−01 | 1 | Unchanged Low | |
| Q9NX47 | K10gh2 | hypothetical protein FLJ20445 | 299.3918 | 338 | 154.4327 | 263.9291 | −9.55E−01 | 1.75E−01 | 1 | Unchanged Low | |
| O60612 | K11cd2 | supervillin | 13.628 | 270 | 154.4497 | 179.421 | 4.43E−01 | 1.25E+00 | 1 | Unchanged Low | |
| O00559 | F22cd4 | estrogen receptor binding site as | 181.5726 | 147 | 154.679 | 160.9495 | −2.31E−01 | −3.09E−01 | 1 | Unchanged Low | |
| O9NUU9 | E17gh3 | hypothetical protein F23149_1 | 140.3785 | 224 | 154.7106 | 172.9723 | 1.40E−01 | 6.73E−01 | 1 | Unchanged Low | |
| P22059 | C10ab8 | oxysterol binding protein | 133.2924 | 93.6 | 154.8714 | 127.2507 | 2.16E−01 | −5.10E−01 | 1 | Unchanged Low | |
| O00408 | I05ef6 | phosphodiesterase 2A: cGMP-sti | 860.2983 | 169 | 154.9688 | 394.9107 | −2.47E+00 | −2.346+00 | 1 | Unchanged Low | |
| O94935 | P22ef7 | SAC1 suppressor of actin mutati | 146.0417 | 128 | 154.9914 | 142.9113 | 6.58E−02 | −1.94E−01 | 1 | Unchanged Low | |
| P41134 | H15ef5 | inhibitor of DNA binding 1; domi | 434.1844 | 292 | 155.0439 | 293.8674 | −1.49E+00 | −5.70E−01 | 1 | Unchanged Low | |
| P24407 | M08ab7 | mel transforming oncogene (deri | 224.1153 | 189 | 155.1684 | 189.5598 | −5.30E−01 | −2.43E−01 | 1 | Unchanged Low | |
| O00232 | N05ab8 | proteasome (prosome; macropai | 122.517 | 139 | 155.2465 | 139.0276 | 3.42E−01 | 1.85E−01 | 1 | Unchanged Low | |
| Q01432 | G04ab2 | adenosine monophosphate dea | 111.1768 | 117 | 155.341 | 127.7664 | 4.83E−01 | 7.10E−02 | 1 | Unchanged Low | |
| Q13525 | G15cd4 | interleukin 1 receptor-like 2 | 136.0802 | 130 | 155.4674 | 140.5051 | 1.92E−01 | −6.636−02 | 1 | Unchanged Low | |
| Q16611 | N04ab3 | BCL2-antagonist/killer 1 | 280.4131 | 194 | 155.5414 | 210.0516 | −8.50E−01 | −5.30E−01 | 1 | Unchanged Low | |
| Q9NPA3 | C11gh5 | hypothetical protein STRAIT1149 | 112.5378 | 85.5 | 155.5531 | 117.8671 | 4.67E−01 | −3.96E−01 | 1 | Unchanged Low | |
| Q9GZM8 | D14gh7 | LIS1-interacting protein NUDEL; | 137.8235 | 131 | 155.5611 | 141.4354 | 1.75E−01 | −7.416−02 | 1 | Unchanged Low | |
| P42702 | P06ab6 | leukemia inhibitory factor recepto | 92.44914 | 112 | 155.6189 | 120.0363 | 7.51E−01 | 2.77E−01 | 1 | Unchanged Low | |
| Q05940 | P07ef5 | solute carrier family 18 (vesicula | 374.4901 | 153 | 155.637 | 227.6447 | −1.27E+00 | −1.296+00 | 1 | Unchanged Low | |
| Q9Y3Q5 | H18cd6 | putative DNA/chromatin binding | 75.96908 | 150 | 155.6726 | 127.0621 | 1.04E+00 | 9.77E−01 | 1 | Unchanged Low | |
| Q15756 | H08ab7 | potassium inwardly-rectifying cha | 149.0689 | 80.9 | 155.7504 | 128.583 | 6.33E−02 | −8.81E−01 | 1 | Unchanged Low | |
| O75386 | M17cd3 | tubby like protein 3 | 128.6914 | 82.7 | 155.7811 | 122.3782 | 2.76E−01 | −6.39E−01 | 1 | Unchanged Low | |
| P50750 | D03ef5 | cyclin-dependent kinase 9 (CDC: | 270.8229 | 215 | 155.8317 | 213.7777 | −7.97E−01 | −3.35E−01 | 1 | Unchanged Low | |
| Q9NWY6 | A18gh3 | hypothetical protein FLJ20531 | 91.85165 | 130 | 156.1332 | 125.9903 | 7.65E−01 | 5.01E−01 | 1 | Unchanged Low | |
| P50391 | K05cd1 | pancreatic polypeptide receptor | 136.5278 | 112 | 156.1953 | 134.9143 | 1.94E−01 | −2.85E−01 | 1 | Unchanged Low | |
| Q9NX07 | L20gh6 | tRNA selenocysteine associated | 258.434 | 287 | 156.2449 | 233.9363 | −7.26E−01 | 1.52E−01 | 1 | Unchanged Low | |
| O75354 | A15ab4 | ectonucleoside triphosphate diph | 386.4989 | 258 | 156.4638 | 267.133 | −1.30E+00 | −5.81E−01 | 1 | Unchanged Low | |
| Q9H832 | L12gh5 | hypothetical protein FLJ13855 | 173.4437 | 307 | 156.9292 | 212.3427 | −1.44E−01 | 8.22E−01 | 1 | Unchanged Low | |
| Q9UBW8 | F18ef1 | COP9 constitutive photomorphog | 102.8052 | 111 | 157.0119 | 123.4686 | 6.11E−01 | 1.05E−01 | 1 | Unchanged Low | |
| P14136 | E23ab5 | gtial fibrillary acidic protein | 108.166 | 86.6 | 157.2761 | 117.3353 | 5.40E−01 | −3.21E−01 | 1 | Unchanged Low | |
| Q9NYF3 | F08ef1 | chromosome 5 open reading fra | 121.4116 | 94.3 | 157.5806 | 124.4392 | 3.76E−01 | −3.64E−01 | 1 | Unchanged Low | |
| Q15056 | A09gh2 | Williams-Beuren syndrome chror | 202.4969 | 124 | 157.6763 | 161.3602 | −3.61E−01 | −7.09E−01 | 1 | Unchanged Low | |
| O15534 | I16ab8 | period homolog 1 (Drosophila) | 237.2007 | 143 | 157.7195 | 179.3665 | −5.89E−01 | −7.28E−01 | 1 | Unchanged Low | |
| Q9Y399 | O21ef2 | mitochondrial ribosomal protein S | 229.9134 | 139 | 157.7339 | 175.5893 | −5.44E−01 | −7.25E−01 | 1 | Unchanged Low | |
| O14972 | A04cd6 | Down syndrome critical region ge | 230.0837 | 267 | 157.7386 | 218.3678 | −5.45E−01 | 2.16E−01 | 1 | Unchanged Low | |
| Q14113 | M15ab2 | AE binding protein 1 | 124.9796 | 95.3 | 157.955 | 126.0667 | 3.38E−01 | −3.92E−01 | 1 | Unchanged Low | |
| O95755 | O18cd5 | RAB36; member RAS oncogene | 132.9919 | 97 | 158.0474 | 129.3518 | 2.49E−01 | −4.55E−01 | 1 | Unchanged Low | |
| Q9NPC3 | I09gh4 | enhancer of invasion 10 | 104.2128 | 121 | 158.1023 | 127.8011 | 6.01E−01 | 2.17E−01 | 1 | Unchanged Low | |
| Q9Y6E9 | N24cd7 | sirtuin silent mating type informal | 111.8557 | 153 | 158.2227 | 140.9006 | 5.00E−01 | 4.48E−01 | 1 | Unchanged Low | |
| Q9Y5B8 | D17cd6 | NME7 | 92.69165 | 116 | 158.2444 | 122.4719 | 7.72E−01 | 3.30E−01 | 1 | Unchanged Low | |
| P49642 | I21ab8 | primase: polypeptide 1 (49kD) | 135.2376 | 70.2 | 156.2887 | 121.2327 | 2.27E−01 | −9.47E−01 | 1 | Unchanged Low | |
| O60660 | J04ab2 | ash2 (absent; small: or homeotic | 274.3502 | 177 | 158.3372 | 203.1619 | −7.93E−01 | −6.34E−01 | 1 | Unchanged Low | |
| Q9H251 | O08gh7 | cadherin related 23 | 113.1038 | 184 | 158.4524 | 151.9031 | 4.86E−01 | 7.03E−01 | 1 | Unchanged Low | |
| Q9Y6C2 | A22cd7 | elastin microfibril interface locate | 107.9751 | 92.2 | 158.6648 | 119.6097 | 5.55E−01 | −2.28E−01 | 1 | Unchanged Low | |
| O95825 | D13cd5 | crystallin; zeta (quinone reductas | 144.0183 | 133 | 158.6719 | 145.1487 | 1.40E−01 | −1.17E−01 | 1 | Unchanged Low | |
| Q9UHG3 | J02ef2 | prenylcysteine lyase | 250.3969 | 207 | 158.8419 | 205.2993 | −6.57E−01 | −2.77E−01 | 1 | Unchanged Low | |
| O00629 | J18ab6 | karyopherin alpha 4 (importin alp | 518.8184 | 295 | 156.8458 | 324.141 | −1.71E+00 | −8.16E−01 | 1 | Unchanged Low | |
| Q15397 | B14gh1 | KIAA0020 gene product | 109.8533 | 134 | 158.9595 | 134.3527 | 5.33E−01 | 2.89E−01 | 1 | Unchanged Low | |
| Q14999 | H01gh1 | KIAA0076 gene product | 71.35119 | 173 | 159.0037 | 134.6153 | 1.16E+00 | 1.286+00 | 1 | Unchanged Low | |
| Q12772 | M18cd2 | sterol regulatory element binding | 158.6108 | 94.6 | 159.1986 | 137.4667 | 5.34E−03 | −7.46E−01 | 1 | Unchanged Low | |
| O75150 | F09gh1 | ring finger protein 40 | 129.2351 | 169 | 159.2038 | 152.3442 | 3.01E−01 | 3.84E−01 | 1 | Unchanged Low | |
| O43423 | L07gh6 | acidic (leucine-rich) nuclear phos | 140.3006 | 143 | 159.4372 | 147.5392 | 1.84E−01 | 2.63E−02 | 1 | Unchanged Low | |
| O43311 | D12cd8 | muscleblind-like (Drosophila) | 165.2243 | 99.4 | 159.4681 | 141.3508 | −5.12E−02 | −7.34E−01 | 1 | Unchanged Low | |
| Q9NYX4 | B07ef4 | calcyon; D1 dopamine receptor-i | 207.2791 | 147 | 159.5302 | 171.42 | −3.78E−01 | −4.91E−01 | 1 | Unchanged Low | |
| Q9HD71 | G04gh4 | hypothetical nuclear factor SBB12 | 123.7874 | 140 | 159.5491 | 141.2517 | 3.66E−01 | 1.82E−01 | 1 | Unchanged Low | |
| Q9NZH2 | N03cd8 | replication initiation region protei | 225.0949 | 179 | 159.6113 | 187.9443 | −4.96E−01 | −3.30E−01 | 1 | Unchanged Low | |
| Q9NVH1 | B05gh3 | hypothetical protein FLJ10737 | 137.4673 | 217 | 159.6034 | 171.2625 | 2.16E−01 | 6.57E−01 | 1 | Unchanged Low | |
| Q9NVG3 | B11gh3 | hypothetical protein FLJ10751 | 96.28296 | 105 | 159.6829 | 120.1783 | 7.30E−01 | 1.19E−01 | 1 | Unchanged Low | |
| P52179 | A21cd4 | myomesin 1 (skelemin) (185kD) | 157.0608 | 94 | 159.8272 | 136.9676 | 2.52E−02 | −7.40E−01 | 1 | Unchanged Low | |
| O94815 | B09cd3 | frizzled homolog 1 (Drosophila) | 93.97877 | 101 | 159.9198 | 118.2939 | 7.67E−01 | 1.04E−01 | 1 | Unchanged Low | |
| Q9UBY9 | G01ef4 | heat shock 27kD protein family: | 98.97552 | 102 | 160.1725 | 120.3216 | 6.94E−01 | 4.08E−02 | 1 | Unchanged Low | |
| Q9NVX7 | G18gh3 | hypothetical protein FLJ10450 | 111.5196 | 121 | 160.2507 | 130.905 | 5.23E−01 | 1.17E−01 | 1 | Unchanged Low | |
| O95704 | A02cd6 | FE65-like protein 2 | 99.35088 | 115 | 160.3239 | 124.7475 | 6.90E−01 | 2.06E−01 | 1 | Unchanged Low | |
| P19801 | A23ab2 | amiloride binding protein 1 (amin | 164.6296 | 68.3 | 160.347 | 131.0836 | −3.80E−02 | −1.27E+00 | 1 | Unchanged Low | |
| O43709 | L18ef3 | Williams Beuren syndrome chror | 127.8538 | 134 | 160.4415 | 140.7865 | 3.28E−01 | 6.84E−02 | 1 | Unchanged Low | |
| Q12839 | N24ef1 | H326 | 109.677 | 116 | 160.6141 | 128.8096 | 5.50E−01 | 8.26E−02 | 1 | Unchanged Low | |
| O43503 | D10ab8 | RAD51 homolog C (S. cerevisiae | 96.34779 | 97.6 | 160.6551 | 118.1934 | 7.38E−01 | 1.83E−02 | 1 | Unchanged Low | |
| Q07898 | J10cd4 | CD163 antigen | 87.73563 | 177 | 160.8614 | 141.7231 | 8.75E−01 | 1.01E+00 | 1 | Unchanged Low | |
| O96015 | F22cd5 | dynein: axonemal; light polypepti | 94.54055 | 251 | 160.9963 | 168.8465 | 7.68E−01 | 1.41E+00 | 1 | Unchanged Low | |
| Q92524 | L11ab6 | proteasome (prosome; macropai | 396.6515 | 220 | 161.0391 | 259.1219 | −1.30E+00 | −8.52E−01 | 1 | Unchanged Low | |
| Q92692 | B06ab8 | poliovirus receptor-related 2 (her | 302.7185 | 317 | 161.2569 | 260.2905 | −9.09E−01 | 6.60E−02 | 1 | Unchanged Low | |
| Q9Y6Y8 | I16cd7 | Sec23-interacting protein p125 | 104.2429 | 131 | 161.2684 | 132.2965 | 6.30E−01 | 3.34E−01 | 1 | Unchanged Low | |
| Q9NZ78 | J16gh3 | uncharacterized bone marrow pr | 107.2001 | 127 | 161.3587 | 131.7449 | 5.90E−01 | 2.41E−01 | 1 | Unchanged Low | |
| Q15120 | G15cd1 | pyruvate dehydrogenase kinase; | 96.64061 | 107 | 161.6468 | 121.6758 | 7.42E−01 | 1.43E−01 | 1 | Unchanged Low | |
| P35241 | F22ab8 | radixin | 276.6012 | 213 | 161.7003 | 216.9693 | −7.74E−01 | −3.80E−01 | 1 | Unchanged Low | |
| Q9Y326 | D23ef2 | brain specific protein | 359.3582 | 160 | 161.7123 | 227.0406 | −1.15E+00 | −1.17E+00 | 1 | Unchanged Low | |
| Q92600 | J03cd5 | RCD1 required for cell differentia | 115.6633 | 145 | 161.7142 | 140.923 | 4.84E−01 | 3.30E−01 | 1 | Unchanged Low | |
| O00487 | N14cd5 | 26S proteasome-associated pad | 329.3518 | 275 | 161.8834 | 255.4035 | −1.02E+00 | −2.60E−01 | 1 | Unchanged Low | |
| Q9Y689 | P21cd7 | ADP-ribosylation factor-like 5 | 137.7737 | 159 | 161.8965 | 152.9706 | 2.33E−01 | 2.09E−01 | 1 | Unchanged Low | |
| Q9ULB8 | P05cd8 | CMP-NeuAC:(beta)-N-acetylgala | 91.01128 | 121 | 161.9148 | 124.5785 | 8.31E−01 | 4.09E−01 | 1 | Unchanged Low | |
| O60897 | L09cd7 | RAB; member of RAS oncogene | 89.32474 | 112 | 162.0127 | 120.9939 | 8.59E−01 | 3.22E−01 | 1 | Unchanged Low | |
| P51809 | J05cd2 | synaptobrevin-like 1 | 228.3546 | 141 | 162.1052 | 177.2043 | −4.94E−01 | −6.94E−01 | 1 | Unchanged Low | |
| O75581 | A03ab7 | low density lipoprotein receptor-r | 122.4708 | 125 | 162.1159 | 136.4067 | 4.05E−01 | 2.53E−02 | 1 | Unchanged Low | |
| O43795 | F12ef4 | myosin class I; myh-1c | 259.4543 | 282 | 162.137 | 234.504 | −6.78E−01 | 1.20E−01 | 1 | Unchanged Low | |
| Q14188 | H17ef5 | transcription factor Dp-2 (E2F dir | 107.308 | 204 | 162.1489 | 157.7494 | 5.96E−01 | 9.25E−01 | 1 | Unchanged Low | |
| P06280 | B07ab3 | galactosidase; alpha | 178.0238 | 304 | 162.2871 | 214.6169 | −1.34E−01 | 7.70E−01 | 1 | Unchanged Low | |
| Q14541 | O17ab7 | hepatocyte nuclear factor 4: gam | 549.8997 | 283 | 162.4659 | 331.895 | −1.76E+00 | −9.57E−01 | 1 | Unchanged Low | |
| P57723 | E17gh8 | poly(rC) binding protein 4 | 186.2845 | 231 | 182.5267 | 193.1283 | −1.97E−01 | 3.08E−01 | 1 | Unchanged Low | |
| Q9NWU3 | A24gh3 | DEAD/H (Asp-Glu-Ala-Asp/His) t | 154.71 | 104 | 162.6529 | 140.5056 | 7.22E−02 | −5.71E−01 | 1 | Unchanged Low | |
| Q9NPC5 | J16ef8 | nudix (nucleoside diphosphate li | 107.1863 | 98.4 | 162.6929 | 122.7442 | 6.02E−01 | −1.24E−01 | 1 | Unchanged Low | |
| Q9Y3C5 | B19ef3 | ring finger protein 11 | 160.0631 | 164 | 162.7518 | 162.3366 | 2.40E−02 | 3.68E−02 | 1 | Unchanged Low | |
| Q9NWX3 | B01gh2 | chromosome 20 open reading fra | 98.58482 | 95.6 | 162.7744 | 118.9844 | 7.23E−01 | −4.44E−02 | 1 | Unchanged Low | |
| P52742 | I22cd3 | zinc finger protein 135 (clone pH | 81.4141 | 121 | 162.8382 | 121.6363 | 1.00E+00 | 5.68E−01 | 1 | Unchanged Low | |
| Q9Y313 | D17ef2 | PTD013 protein | 90.25232 | 116 | 163.4053 | 123.3545 | 8.56E−01 | 3.67E−01 | 1 | Unchanged Low | |
| Q9P2W9 | B11ef4 | syntaxin 18 | 128.6426 | 188 | 163.4277 | 160.0555 | 3.45E−01 | 5.48E−01 | 1 | Unchanged Low | |
| P14778 | F09ab6 | interleukin 1 receptor; type I | 264.5401 | 539 | 163.4432 | 322.4754 | −6.95E−01 | 1.03E+00 | 1 | Unchanged Low | |
| Q15464 | I12ef5 | SHB (Src homology 2 domain co | 284.291 | 301 | 163.5379 | 249.6096 | −7.98E−01 | 8.24E−02 | 1 | Unchanged Low | |
| O15444 | H07cd2 | small inducible cytokine subfamil | 244.6426 | 97.1 | 163.6053 | 168.4558 | −5.80E−01 | −1.33E+00 | 1 | Unchanged Low | |
| P49796 | P14ef4 | regulator of G-protein signalling: | 220.2769 | 99.3 | 163.6222 | 161.0774 | −4.29E−01 | −1.15E+00 | 1 | Unchanged Low | |
| Q9NUS7 | B04gh3 | hypothetical protein FLJ11164 | 81.76083 | 114 | 163.6422 | 119.7258 | 1.00E+00 | 4.77E−01 | 1 | Unchanged Low | |
| P48651 | L11ef3 | phosphatidylserine synthase 1 | 211.8695 | 139 | 163.6792 | 171.5708 | −3.72E−01 | −6.06E−01 | 1 | Unchanged Low | |
| Q07706 | F15cd4 | tetracycline transporter-like prote | 143.0791 | 144 | 163.7206 | 150.2019 | 1.94E−01 | 7.31E−03 | 1 | Unchanged Low | |
| O75143 | B11gh1 | KIAA0652 gene product | 106.5331 | 155 | 163.7687 | 141.6405 | 6.20E−01 | 5.37E−01 | 1 | Unchanged Low | |
| O75039 | L19gh1 | KIAA0451 gene product | 174.142 | 135 | 163.8572 | 157.7714 | −8.78E−02 | −3.64E−01 | 1 | Unchanged Low | |
| P02261 | M10gh6 | H2A histone family; member N | 93.48591 | 273 | 163.8621 | 176.9259 | 8.10E−01 | 1.55E+00 | 1 | Unchanged Low | |
| Q9H9A0 | L20gh5 | hypothetical protein FLJ12895 | 86.42599 | 130 | 164.3655 | 126.7707 | 9.27E−01 | 5.84E−01 | 1 | Unchanged Low | |
| Q9NV35 | J21gh3 | hypothetical protein FLJ10956 | 99.15342 | 154 | 164.4852 | 139.1489 | 7.30E−01 | 6.33E−01 | 1 | Unchanged Low | |
| Q9P1G8 | C05gh4 | WW domain-containing adapter | 119.4436 | 182 | 164.5311 | 155.3256 | 4.62E−01 | 6.08E−01 | 1 | Unchanged Low | |
| P26368 | D11cd7 | U2 small nuclear ribonucleoprote | 217.0564 | 107 | 164.8861 | 163.126 | −3.97E−01 | −1.01E+00 | 1 | Unchanged Low | |
| Q9BSZ7 | E11gh8 | DEAD/H (Asp-Glu-Ala-Asp/His) | 130.7858 | 111 | 164.9084 | 135.6059 | 3.34E−01 | −2.35E−01 | 1 | Unchanged Low | |
| Q9Y391 | G23ef2 | androgen-regulated short-chain | 222.3497 | 128 | 164.9823 | 171.7067 | −4.31E−01 | 7.99E−01 | 1 | Unchanged Low | |
| Q9NYR5 | E05ef3 | HSPC126 protein | 99.37243 | 110 | 165.2098 | 124.8579 | 7.33E−01 | 1.46E−01 | 1 | Unchanged Low | |
| P05165 | N12ab7 | propionyl Coenzymn A carboxyla | 100.892 | 120 | 165.6902 | 128.9755 | 7.16E−01 | 2.54E−01 | 1 | Unchanged Low | |
| Q9NPF4 | O21gh3 | O-sialoglycoprotein endopeptidas | 103.8223 | 112 | 165.9716 | 127.3658 | 6.77E−01 | 1.13E−01 | 1 | Unchanged Low | |
| P16106 | D01cd3 | H3 histone family; member K | 99.19006 | 130 | 166.1216 | 131.6683 | 7.44E−01 | 3.87E−01 | 1 | Unchanged Low | |
| P35249 | O03ef5 | replication factor C (activator 1) | 105.2406 | 98.7 | 166.2092 | 123.3812 | 6.59E−01 | −9.27E−02 | 1 | Unchanged Low | |
| P28702 | P18gh1 | retinoid X receptor; beta | 74.914 | 142 | 166.2214 | 127.7227 | 1.15E+00 | 9.23E−01 | 1 | Unchanged Low | |
| P49674 | K14ab4 | casein kinase 1; epsilon | 99.33956 | 145 | 166.3564 | 136.8564 | 7.44E−01 | 5.44E−01 | 1 | Unchanged Low | |
| Q9NXW2 | I03gh2 | DnaJ (Hsp40) homolog; subfamil | 247.7907 | 235 | 166.4412 | 216.5041 | −5.74E−01 | −7.47E−02 | 1 | Unchanged Low | |
| O14672 | I01ab2 | a disintegrin and metalloproteina | 378.0042 | 226 | 166.5417 | 256.8185 | −1.18E+00 | −7.43E−01 | 1 | Unchanged Low | |
| Q9Y5B9 | I20cd7 | chromatin-specific transcription e | 163.0543 | 188 | 166.6045 | 172.4726 | 3.11E−02 | 2.04E−01 | 1 | Unchanged Low | |
| Q9H9J2 | H22gh5 | mitochondrial ribosomal protein | 206.428 | 256 | 166.8142 | 209.7386 | −3.07E−01 | 3.10E−01 | 1 | Unchanged Low | |
| P23759 | G06ab8 | paired box gene 7 | 92.41833 | 200 | 166.8514 | 153.0178 | 8.52E−01 | 1.11E+00 | 1 | Unchanged Low | |
| Q16643 | L05ab4 | drebrin 1 | 265.008 | 152 | 166.9734 | 194.7352 | −6.66E−01 | −8.00E−01 | 1 | Unchanged Low | |
| P48454 | I21cd1 | protein phosphatase 3 (formerly: | 105.9496 | 169 | 167.0428 | 147.2674 | 6.57E−01 | 6.72E−01 | 1 | Unchanged Low | |
| Q9Y508 | F21ef4 | zinc finger protein 313 | 331.595 | 395 | 167.2366 | 297.9139 | −9.88E−01 | 2.52E−01 | 1 | Unchanged Low | |
| P19474 | G09cd2 | Sjogren syndrome antigen A1 (52 | 301.1776 | 163 | 167.414 | 217.1349 | −8.47E−01 | −7.20E−01 | 1 | Unchanged Low | |
| P46109 | N13ab5 | v-crk sarcoma virus CT10 oncog | 151.2065 | 141 | 167.7716 | 153.202 | 1.50E−01 | −1.05E−01 | 1 | Unchanged Low | |
| P54855 | B19cd4 | UDP glycosyltransferase 2 family | 137.8939 | 127 | 167.8346 | 144.2152 | 2.83E−01 | −1.20E−01 | 1 | Unchanged Low | |
| Q14242 | H14cd1 | selectin P ligand | 112.9679 | 130 | 167.9184 | 136.8261 | 5.72E−01 | 1.98E−01 | 1 | Unchanged Low | |
| Q9BSS1 | P17gh8 | hypothetical protein FLJ14525 | 86.75908 | 113 | 167.974 | 122.482 | 9.53E−01 | 3.78E−01 | 1 | Unchanged Low | |
| Q9Y3B2 | L23ef1 | homolog of yeast exosomal core | 152.0751 | 140 | 167.9915 | 153.2264 | 1.44E−01 | −1.23E−01 | 1 | Unchanged Low | |
| P31391 | N03cd1 | somatostatin receptor 4 | 147.3942 | 92.5 | 168.0254 | 135.9754 | 1.89E−01 | −6.72E−01 | 1 | Unchanged Low | |
| O60623 | O03ab4 | diptheria toxin resistance protein | 210.9658 | 170 | 168.0762 | 183.0009 | −3.28E−01 | −3.12E−01 | 1 | Unchanged Low | |
| Q99959 | L24ab8 | plakophilin 2 | 362.9423 | 211 | 168.1655 | 247.4823 | −1.11E+00 | −7.80E−01 | 1 | Unchanged Low | |
| | B21gh7 | leukocyte immunoglobulin-like re | 139.2571 | 234 | 168.1829 | 180.4876 | 2.72E−01 | 7.49E−01 | 1 | Unchanged Low | |
| Q01658 | P18ab5 | down-regulator of transcription 1 | 205.2249 | 122 | 168.2606 | 165.0596 | −2.87E−01 | −7.54E−01 | 1 | Unchanged Low | |
| Q9HCS4 | P10gh7 | HMG-box transcription factor TC | 76.46423 | 156 | 168.2926 | 133.5936 | 1.14E+00 | 1.03E+00 | 1 | Unchanged Low | |
| Q9P035 | N20ef2 | butyrate-induced transcript 1 | 102.716 | 142 | 168.3034 | 137.5999 | 7.12E−01 | 4.65E−01 | 1 | Unchanged Low | |
| Q9C0K7 | P24gh3 | amyotrophic lateral sclerosis 2 (j | 102.352 | 108 | 168.4355 | 126.1345 | 7.19E−01 | 7.24E−02 | 1 | Unchanged Low | |
| Q9NYF4 | I13ef3 | chromosome 5 open reading fra | 89.72908 | 137 | 168.4424 | 131.7509 | 9.09E−01 | 6.11E−01 | 1 | Unchanged Low | |
| P30042 | M19cd4 | chromosome 21 open reading fra | 98.84241 | 97.5 | 168.5171 | 121.6076 | 7.70E−01 | −2.03E−02 | 1 | Unchanged Low | |
| O75325 | K16cd6 | glioma amplified on chromosome | 116.3239 | 83.3 | 168.5172 | 122.705 | 5.35E−01 | −4.82E−01 | 1 | Unchanged Low | |
| Q9Y670 | N01ef2 | transient receptor potential catio | 96.41826 | 90.8 | 168.5297 | 118.5764 | 8.06E−01 | −8.69E−02 | 1 | Unchanged Low | |
| Q9UPR8 | I14ef8 | KIAA1084 protein | 101.3235 | 111 | 168.5336 | 126.797 | 7.34E−01 | 1.26E−01 | 1 | Unchanged Low | |
| Q9Y2X7 | I21ef4 | G protein-coupled receptor kinas | 160.7403 | 167 | 168.7352 | 165.5706 | 7.00E−02 | 5.72E−02 | 1 | Unchanged Low | |
| P47872 | D24cd1 | secretin receptor | 174.6672 | 209 | 168.738 | 184.1802 | −4.98E−02 | 2.60E−01 | 1 | Unchanged Low | |
| O43353 | C17cd4 | receptor-interacting serine-threo | 272.5279 | 388 | 168.7722 | 276.5664 | −6.91E−01 | 5.11E−01 | 1 | Unchanged Low | |
| P32321 | P14ab5 | dCMP deaminase | 218.5685 | 60.1 | 168.8404 | 149.1554 | −3.72E−01 | −1.86E+00 | 1 | Unchanged Low | |
| O43612 | J16ab4 | hypocretin (orexin) neuropeptide | 66.45691 | 194 | 168.9064 | 143.2577 | 1.35E+00 | 1.55E+00 | 1 | Unchanged Low | |
| Q92874 | M15ab4 | deoxyribonuclease I-like 2 | 218.1787 | 146 | 168.9596 | 177.8201 | −3.69E−01 | −5.76E−01 | 1 | Unchanged Low | |
| Q9U190 | I15ef8 | PRO0036 protein | 89.17218 | 389 | 168.9849 | 215.81 | 9.22E−81 | 2.12E+00 | 1 | Unchanged Low | |
| P30519 | N05ab6 | heme oxygenase (decycling) 2 | 586.3897 | 387 | 169.1269 | 380.9032 | −1.79E+00 | −5.99E−01 | 1 | Unchanged Low | |
| Q9NVE7 | D03gh3 | hypothetical protein FLJ10782 | 141.6695 | 148 | 189.2644 | 152.9293 | 2.57E−01 | 6.16E−02 | 1 | Unchanged Low | |
| Q9P010 | A14ef8 | HSPC154 protein | 227.9143 | 246 | 169.4393 | 214.317 | −4.28E−01 | 1.08E−01 | 1 | Unchanged Low | |
| Q9UH59 | M04cd8 | bromodomain-containing 7 | 147.6635 | 103 | 189.5045 | 139.9456 | 1.99E−01 | −5.24E−01 | 1 | Unchanged Low | |
| Q9UM82 | E01cd6 | spermatogenesis associated 2 | 230.7114 | 221 | 169.6678 | 207.1296 | −4.43E−01 | −6.20E−02 | 1 | Unchanged Low | |
| Q9P2Y4 | N14ef1 | zinc finger protein 219 | 105.6753 | 175 | 169.7666 | 150.1216 | 6.84E−81 | 7.27E−01 | 1 | Unchanged Low | |
| Q9GZS3 | H19gh7 | recombination protein REC14 | 124.7245 | 162 | 169.9984 | 152.3724 | 4.47E−01 | 3.81E−01 | 1 | Unchanged Low | |
| P07686 | J17ab3 | hexosaminidase B (beta polypep | 217.0423 | 135 | 170.0092 | 174.0094 | −3.52E−01 | −6.85E−01 | 1 | Unchanged Low | |
| Q9NZV6 | L06ef2 | selenoprotein X: 1 | 241.3633 | 202 | 170.0232 | 204.3052 | −5.05E−01 | −2.60E−01 | 1 | Unchanged Low | |
| O14678 | E17cd1 | ATP-binding cassette; sub-family | 77.65216 | 127 | 170.0525 | 124.9148 | 1.13E+00 | 7.10E−01 | 1 | Unchanged Low | |
| P29972 | O04ab2 | aquaporin 1 (channel-forming int | 309.1218 | 134 | 170.2078 | 204.2885 | −8.61E−01 | −1.21E+00 | 1 | Unchanged Low | |
| O95373 | D03cd6 | RAN binding protein 7 | 247.4368 | 113 | 170.45 | 177.0459 | −5.38E−01 | −1.13E+00 | 1 | Unchanged Low | |
| Q15436 | O12cd6 | Sec23 homolog A (S. cerevisiae) | 90.05071 | 100 | 170.9386 | 120.3545 | 9.25E−01 | 1.52E−01 | 1 | Unchanged Low | |
| Q9BQ67 | K23gh8 | glutamate rich WD repeat protein | 140.3198 | 112 | 170.966 | 141.0924 | 2.85E−01 | −3.25E−01 | 1 | Unchanged Low | |
| P35321 | D21gh6 | smutt proline-rich protein 1A | 62.28271 | 237 | 171.0104 | 156.8515 | 1.46E+00 | 1.93E+00 | 1 | Unchanged Low | |
| Q9UPQ8 | I10ef8 | KIAA1094 protein | 76.76245 | 123 | 171.0467 | 123.4833 | 1.16E+00 | 6.76E−01 | 1 | Unchanged Low | |
| O43292 | A19cd4 | GPAA1P anchor attachment prot | 95.36294 | 92.4 | 171.0704 | 119.6038 | 8.43E−01 | −4.59E−02 | 1 | Unchanged Low | |
| Q9UBM3 | G02cd1 | proteasome (prosome; macropai | 287.6026 | 204 | 171.1554 | 221.0285 | −7.49E−01 | −4.93E−01 | 1 | Unchanged Low | |
| P19338 | M20ab7 | nucleolin | 148.8642 | 87.6 | 171.2094 | 135.8947 | 2.02E−01 | −7.65E−01 | 1 | Unchanged Low | |
| Q9NPE6 | E17gh1 | sperm associated antigen 4 | 111.172 | 135 | 171.4356 | 139.3341 | 6.25E−01 | 2.84E−01 | 1 | Unchanged Low | |
| O00442 | J12cd3 | RTC domain containing 1 | 274.6701 | 279 | 171.531 | 241.6967 | −6.79E−01 | 2.20E−02 | 1 | Unchanged Low | |
| P28356 | D09ef1 | homeo box D9 | 84.5638 | 106 | 171.5949 | 120.5873 | 1.02E+00 | 3.21E−01 | 1 | Unchanged Low | |
| P49447 | G06ef1 | cytochrome b-561 | 174.7152 | 167 | 171.6447 | 171.1671 | −2.56E−02 | −6.39E−02 | 1 | Unchanged Low | |
| Q9UQA4 | P18cd4 | nuclear factor (erythroid-derived | 227.4253 | 470 | 171.8945 | 289.7702 | −4.04E−01 | 1.05E+00 | 1 | Unchanged Low | |
| O15254 | D09ab2 | acyl-Coenzyme A oxidase 3: pris | 192.2301 | 192 | 172.0218 | 185.2853 | −1.60E−01 | −4.71E−03 | 1 | Unchanged Low | |
| Q9NWW6 | B11gh2 | hypothetical protein FLJ20559 | 198.7861 | 220 | 172.0261 | 197.0098 | −2.09E−01 | 1.48E−01 | 1 | Unchanged Low | |
| O75391 | M08cd5 | sperm associated antigen 7 | 154.1159 | 140 | 172.0658 | 155.2376 | 1.59E−01 | −1.43E−01 | 1 | Unchanged Low | |
| Q92959 | H19cd2 | solute carrier family 21 (prostagl | 86.25465 | 102 | 172.112 | 120.1434 | 9.97E−01 | 2.43E−01 | 1 | Unchanged Low | |
| O00499 | G06ab2 | bridging integrator 1 | 162.8584 | 111 | 172.4453 | 148.6447 | 8.25E−02 | −5.50E−01 | 1 | Unchanged Low | |
| Q14197 | J05ab6 | immature colon carcinoma trans | 90.92583 | 102 | 172.5526 | 121.725 | 9.24E−01 | 1.62E−01 | 1 | Unchanged Low | |
| Q9NXS9 | K15gh2 | hypothetical protein FLJ20071 | 121.4575 | 103 | 172.5552 | 132.3749 | 5.07E−01 | −2.36E−01 | 1 | Unchanged Low | |
| O75954 | N18cd6 | tetraspan tranemembrane 4 supe | 83.06795 | 119 | 172.5557 | 124.8565 | 1.05E+00 | 5.18E−01 | 1 | Unchanged Low | |
| Q9UJY9 | E17cd8 | lifeguard | 156.5909 | 75.6 | 172.6495 | 134.9529 | 1.41E−01 | −1.05E+00 | 1 | Unchanged Low | |
| P82912 | F10gh5 | mitochondrial ribosomal protein S | 105.0578 | 196 | 172.7091 | 156.0886 | 7.17E−01 | 9.03E−01 | 1 | Unchanged Low | |
| Q12918 | J02ab6 | killer cell lectin-like receptor subf | 205.6225 | 290 | 172.8141 | 222.8453 | −2.51E−01 | 4.97E−01 | 1 | Unchanged Low | |
| Q14789 | E01ab6 | golgi autoantigen; golgin subfam | 96.09162 | 194 | 173.0059 | 154.2154 | 8.48E−01 | 1.01E+00 | 1 | Unchanged Low | |
| Q04206 | I23ef6 | v-rel reticuloendotheliosis viral o | 328.9295 | 242 | 173.0443 | 248.1566 | −9.27E−01 | −4.40E−01 | 1 | Unchanged Low | |
| P19838 | M15ef5 | nuclear factor of kappa light poly | 229.2262 | 277 | 173.0997 | 226.323 | −4.05E−01 | 2.71E−01 | 1 | Unchanged Low | |
| Q92748 | A24cd2 | thyroid hormone responsive (SP | 101.3906 | 88.2 | 173.314 | 120.9562 | 7.73E−01 | −2.02E−01 | 1 | Unchanged Low | |
| O95095 | P13ab2 | amyloid beta precursor protein ( | 199.951 | 201 | 173.3314 | 191.35 | −2.06E−01 | 5.88E−03 | 1 | Unchanged Low | |
| P52209 | K18ab8 | phosphogluconate dehydrogenas | 211.8178 | 147 | 173.42 | 177.5327 | −2.89E−01 | −5.23E−01 | 1 | Unchanged Low | |
| P42566 | H10ef5 | epidermal growth factor receptor | 194.9437 | 158 | 173.4643 | 175.3861 | −1.68E−01 | −3.05E−01 | 1 | Unchanged Low | |
| Q13772 | D05gh6 | nuclear receptor coactivator 4 | 273.5167 | 367 | 173.5038 | 271.4641 | −6.57E−01 | 4.26E−01 | 1 | Unchanged Low | |
| P43897 | D09gh6 | Ts translation elongation factor; | 325.7575 | 226 | 173.8489 | 241.8156 | −9.06E−01 | −5.28E−01 | 1 | Unchanged Low | |
| P09329 | D08ef7 | phosphoribosyl pyrophosphate s | 103.7882 | 86.2 | 174.5738 | 121.5057 | 7.50E−01 | −2.69E−01 | 1 | Unchanged Low | |
| O75618 | F24cd4 | death effector domain-containing | 172.0163 | 176 | 174.7689 | 174.3849 | 2.29E−02 | 3.61E−02 | 1 | Unchanged Low | |
| Q9UHI9 | B18ef1 | flavohemoprotein b5+b5R | 169.2028 | 197 | 174.7919 | 180.2862 | 4.69E−02 | 2.18E−01 | 1 | Unchanged Low | |
| Q9H824 | J07gh6 | F-box only protein 22 | 89.2751 | 161 | 175.62 | 141.9533 | 9.76E−01 | 8.50E−01 | 1 | Unchanged Low | |
| Q9UQ43 | G07ab3 | BAI1-associated protein 2 | 164.8198 | 165 | 175.6399 | 175.1475 | −7.35E−02 | −1.64E−01 | 1 | Unchanged Low | |
| O43173 | N08ef1 | sialyltransferase 8C (alpha2:3Ga | 220.9909 | 256 | 175.9869 | 217.6266 | −3.29E−01 | 2.12E−01 | 1 | Unchanged Low | |
| Q9BZZ2 | G13gh7 | sialoadhesin | 147.52 | 189 | 175.9952 | 170.7942 | 2.55E−01 | 3.56E−01 | 1 | Unchanged Low | |
| Q92611 | Il6gh1 | KIAA0212 gene product | 96.3144 | 94.3 | 176.0348 | 122.2106 | 8.70E−01 | −3.06E−02 | 1 | Unchanged Low | |
| Q969V6 | K14gh7 | megakaryoblastic leukemia (tran | 135.6533 | 161 | 176.0531 | 157.6423 | 3.76E−01 | 2.49E−01 | 1 | Unchanged Low | |
| P08107 | K14ab7 | heat shock 70kD protein 1B | 153.5141 | 106 | 176.1145 | 145.1219 | 1.98E−01 | −5.38E−01 | 1 | Unchanged Low | |
| Q9BQ24 | E21gh6 | hypothetical protein MGC2550 | 153.9741 | 255 | 176.3889 | 195.2182 | 1.96E−01 | 7.29E−01 | 1 | Unchanged Low | |
| Q9H7X1 | N07gh5 | hypothetical protein FLJ14153 | 155.291 | 236 | 176.5938 | 189.2619 | 1.85E−01 | 6.03E−01 | 1 | Unchanged Low | |
| Q99616 | D09cd2 | small inducible cytokine subfamil | 130.1641 | 100 | 176.5958 | 135.6192 | 4.40E−01 | −3.79E−01 | 1 | Unchanged Low | |
| Q92896 | L04cd7 | golgi apparatus protein 1 | 97.73429 | 90.3 | 176.622 | 121.5361 | 8.54E−01 | −1.15E−01 | 1 | Unchanged Low | |
| Q14582 | J16ef6 | Mad4 homolog | 210.3185 | 244 | 176.6383 | 210.1876 | −2.52E−01 | 2.12E−01 | 1 | Unchanged Low | |
| Q13505 | K21ab7 | metaxin 1 | 199.1231 | 168 | 176.6745 | 181.2437 | −1.73E−01 | −2.46E−01 | 1 | Unchanged Low | |
| Q13722 | O24cd6 | chromosome 3p21.1 gene seque | 85.85963 | 144 | 176.7101 | 135.6336 | 1.04E+00 | 7.49E−01 | 1 | Unchanged Low | |
| P01308 | I10ab6 | insulin | 367.022 | 113 | 176.7437 | 218.8465 | −1.05E+00 | −1.70E+00 | 1 | Unchanged Low | |
| O00405 | D19ab8 | protein phosphatase 1; regulator | 233.0866 | 306 | 176.8763 | 238.6383 | −3.98E−01 | 3.92E−01 | 1 | Unchanged Low | |
| P41215 | E03ab5 | fatty-acid-Coenzyme A ligase; loi | 267.9822 | 199 | 177.1301 | 214.8046 | −5.97E−01 | −4.27E−01 | 1 | Unchanged Low | |
| P31314 | F04ef6 | homeo box 11 (T-cell lymphoma | 107.4127 | 170 | 177.4236 | 151.5175 | 7.24E−01 | 6.60E−01 | 1 | Unchanged Low | |
| O95427 | G21cd8 | phosphatidylinositol glycan; class | 84.22817 | 116 | 177.4629 | 125.8189 | 1.08E+00 | 4.59E−01 | 1 | Unchanged Low | |
| Q12981 | C24ef6 | BCL2/adenovirus E1B 19kD inte | 82.38214 | 99.8 | 177.5246 | 119.9152 | 1.11E+00 | 2.77E−01 | 1 | Unchanged Low | |
| Q9Y5V1 | L10ef1 | HSPC042 protein | 212.074 | 184 | 177.7635 | 191.3703 | −2.55E−01 | −2.03E−01 | 1 | Unchanged Low | |
| Q9H4B4 | O22ab4 | cytokine-inducible kinase | 161.982 | 194 | 177.788 | 177.8629 | 1.34E−01 | 2.59E−01 | 1 | Unchanged Low | |
| Q9H6D1 | P15gh5 | chromosome 20 open reading fra | 95.43432 | 108 | 177.8526 | 126.9312 | 8.98E−01 | 1.72E−01 | 1 | Unchanged Low | |
| O60504 | J18cd5 | vinexin beta (SH3-containing ada | 109.3831 | 99.8 | 177.8876 | 129.0088 | 7.02E−01 | −1.33E−01 | 1 | Unchanged Low | |
| Q9UNK0 | I02cd5 | syntaxin 8 | 218.2149 | 171 | 177.9777 | 189.0342 | −2.94E−01 | −3.53E−01 | 1 | Unchanged Low | |
| O95657 | B24cd4 | proline-serine-threonine phospha | 369.9485 | 158 | 178.0072 | 235.2956 | −1.06E+00 | −1.23E+00 | 1 | Unchanged Low | |
| P20645 | C05ab7 | mannose-6-phosphate receptor ( | 355.8249 | 234 | 178.1318 | 256.0667 | −9.98E−01 | −6.03E−01 | 1 | Unchanged Low | |
| O14569 | B03ab2 | putative tumor suppressor | 122.4884 | 79.2 | 178.136 | 126.62 | 5.40E−01 | −6.28E−01 | 1 | Unchanged Low | |
| O15293 | D18cd3 | MAP-kinase activating death don | 157.092 | 114 | 178.1677 | 149.8523 | 1.82E−01 | −4.59E−01 | 1 | Unchanged Low | |
| P00390 | E14ef7 | glutathione reductase | 282.1492 | 196 | 178.1741 | 218.8246 | −6.63E−01 | −5.24E−01 | 1 | Unchanged Low | |
| Q9HBV3 | K24gh5 | PP3111 protein | 229.2714 | 361 | 178.4093 | 256.1517 | −3.62E−01 | 6.54E−01 | 1 | Unchanged Low | |
| Q14558 | H17ab8 | phosphoribosyl pyrophosphate s | 301.219 | 227 | 178.4567 | 235.5213 | −7.55E−01 | −4.09E−01 | 1 | Unchanged Low | |
| O14710 | F01ef5 | cell cycle progression 2 protein | 261.2178 | 189 | 178.7923 | 209.7103 | −5.47E−01 | −4.66E−01 | 1 | Unchanged Low | |
| Q9UJ70 | J10ef3 | N-acetylglucosamine kinase | 151.2986 | 276 | 178.7992 | 201.9381 | 2.41E−01 | 8.66E−01 | 1 | Unchanged Low | |
| Q13397 | M18cd6 | zinc finger protein 238 | 215.883 | 285 | 178.9318 | 226.5893 | −2.71E−01 | 4.00E−01 | 1 | Unchanged Low | |
| Q9NRG4 | B21gh4 | HSKM-B protein | 143.1326 | 279 | 178.999 | 200.3335 | 3.23E−01 | 9.62E−01 | 1 | Unchanged Low | |
| P51449 | B01cd2 | RAR-related orphan receptor C | 236.6977 | 97.2 | 179.0372 | 170.992 | −4.03E−01 | −1.28E+00 | 1 | Unchanged Low | |
| Q9H768 | O21gh7 | synaptotagmin-like 2 | 83.78255 | 121 | 179.0813 | 128.0355 | 1.10E+00 | 5.33E−01 | 1 | Unchanged Low | |
| Q14332 | F22ef5 | frizzled homolog 2 (Drosophila) | 88.25011 | 129 | 179.1323 | 131.3078 | 1.05E+00 | 5.76E−01 | 1 | Unchanged Low | |
| Q13207 | N13cd2 | T-box 2 | 118.9378 | 108 | 179.1858 | 135.2524 | 5.91E−01 | −1.44E−01 | 1 | Unchanged Low | |
| Q9H6B8 | B19gh5 | hypothetical protein similar to m | 134.338 | 129 | 179.5015 | 147.5119 | 4.18E−01 | −6.19E−02 | 1 | Unchanged Low | |
| O00482 | J09ab7 | mannosidase; beta A: lysosomal | 83.23817 | 107 | 179.6953 | 123.2365 | 1.11E+00 | 3.59E−01 | 1 | Unchanged Low | |
| Q9Y3D8 | H14ef2 | adrenal gland protein AD-004 | 178.4931 | 183 | 179.7989 | 173.7578 | 1.05E−02 | −1.31E−01 | 1 | Unchanged Low | |
| P17900 | F19ab3 | GM2 ganglioside activator protein | 124.7747 | 232 | 179.914 | 178.9058 | 5.28E−01 | 8.95E−01 | 1 | Unchanged Low | |
| Q9P0M4 | O10cd8 | interleukin 17C | 260.8753 | 223 | 180.0241 | 221.2511 | −5.35E−01 | −2.27E−01 | 1 | Unchanged Low | |
| | F13gh8 | G protein-coupled receptor 54 | 87.92591 | 133 | 180.0735 | 133.5723 | 1.03E+00 | 5.94E−01 | 1 | Unchanged Low | |
| Q9NZ92 | J02gh3 | SWI/SNF related; matrix associa | 267.7307 | 247 | 180.206 | 231.4963 | −5.71E−01 | −1.19E−01 | 1 | Unchanged Low | |
| Q9NX94 | L18gh6 | hypothetical protein FLJ20154 | 133.6318 | 165 | 180.3424 | 159.7713 | 4.32E−01 | 3.07E−01 | 1 | Unchanged Low | |
| P04049 | G12ef5 | v-raf-1 murine leukemia viral onc | 225.086 | 183 | 180.3925 | 196.2063 | −3.19E−01 | −2.98E−01 | 1 | Unchanged Low | |
| Q13181 | B06cd1 | ATP-binding cassette: sub-family | 167.9383 | 127 | 180.4651 | 158.5133 | 1.04E−01 | −4.02E−01 | 1 | Unchanged Low | |
| P22102 | M05ab6 | phosphoribosylglycinamide formy | 295.0489 | 180 | 180.5026 | 218.4202 | −7.09E−01 | −7.15E−01 | 1 | Unchanged Low | |
| Q9NQZ8 | A09gh5 | endothelial zinc finger protein in | 96.79284 | 138 | 180.7283 | 138.4428 | 9.01E−01 | 5.10E−01 | 1 | Unchanged Low | |
| Q9UN30 | D04cd2 | sex comb on midleg-like 1 (Dros | 97.47885 | 86 | 181.0121 | 121.4921 | 8.93E−01 | −1.81E−01 | 1 | Unchanged Low | |
| P26651 | J04ef6 | zinc finger protein 36: C3H type; | 286.0733 | 162 | 181.0638 | 209.8211 | −6.60E−01 | −8.17E−01 | 1 | Unchanged Low | |
| P18887 | O06ef6 | X-ray repair complementing defe | 85.61601 | 102 | 181.0778 | 122.9254 | 1.08E+00 | 2.54E−01 | 1 | Unchanged Low | |
| Q9Y2S0 | P14ef1 | RNA polymerase I 16kDa subun | 182.9338 | 149 | 181.2184 | 170.8985 | −1.36E−02 | −3.00E−01 | 1 | Unchanged Low | |
| P49023 | I05ef7 | paxillin | 313.7788 | 334 | 181.2995 | 278.3491 | −7.91E−01 | 9.00E−02 | 1 | Unchanged Low | |
| P49321 | O03ab7 | nuclear autoantigenic sperm prol | 119.903 | 78.2 | 181.3524 | 126.4866 | 5.97E−01 | −6.17E−01 | 1 | Unchanged Low | |
| Q13428 | M14cd1 | Treacher Collins-Franceschetti s | 164.3621 | 123 | 181.4282 | 156.2838 | 1.43E−01 | −4.18E−01 | 1 | Unchanged Low | |
| Q9HCS6 | G07gh4 | vacuolar protein sorting 11 (yeas | 177.2833 | 302 | 181.4601 | 220.0848 | 3.36E−02 | 7.66E−01 | 1 | Unchanged Low | |
| P20813 | I02gh6 | cytochrome P450; subfamily IIB ( | 122.3562 | 162 | 181.476 | 155.3947 | 5.69E−01 | 4.08E−01 | 1 | Unchanged Low | |
| O00459 | C05cd1 | phosphoinositide-3-kinase; regul | 145.3741 | 160 | 181.534 | 182.3275 | 3.20E−01 | 1.39E−01 | 1 | Unchanged Low | |
| P50151 | H24ab4 | guanine nucleotide binding prote | 186.5329 | 193 | 182.0963 | 187.0796 | −3.47E−02 | 4.62E−02 | 1 | Unchanged Low | |
| Q16850 | D12ab3 | cytochrome P450: 51 (lanosterol | 353.9667 | 116 | 182.1309 | 217.3331 | −9.59E−01 | −1.61E+00 | 1 | Unchanged Low | |
| Q9ULW3 | J09cd8 | TATA-binding protein-binding pr | 97.23454 | 207 | 182.1565 | 162.0616 | 9.06E−01 | 1.09E+00 | 1 | Unchanged Low | |
| Q16378 | O20cd7 | proline rich 4 (lacrimal) | 81.91 | 152 | 182.3589 | 138.8678 | 1.15E+00 | 8.95E−01 | 1 | Unchanged Low | |
| P41240 | J14ab4 | c-src tyrosine kinase | 175.1285 | 153 | 182.4257 | 170.0806 | 5.89E−02 | −1.98E−01 | 1 | Unchanged Low | |
| Q16649 | G05cd1 | nuclear factor: inlerleukin 3 regul | 353.6698 | 169 | 182.4266 | 235.1072 | −9.55E−01 | −1.06E+00 | 1 | Unchanged Low | |
| Q9Y3M2 | J03ef8 | chromosome 22 open reading fra | 74.27488 | 194 | 182.6022 | 150.2264 | 1.30E+00 | 1.38E+00 | 1 | Unchanged Low | |
| Q9UK58 | M14gh4 | cyclic L ania-6a | 191.3619 | 313 | 182.6351 | 229.0134 | −6.73E−02 | 7.10E−01 | 1 | Unchanged Low | |
| P47874 | M23cd1 | olfactory marker protein | 250.3691 | 172 | 182.73 | 201.6274 | −4.54E−01 | −5.43E−01 | 1 | Unchanged Low | |
| O95620 | O03cd7 | protein similar to E. coli yhdg and | 227.8932 | 207 | 183.1055 | 205.8925 | −3.16E−01 | −1.41E−01 | 1 | Unchanged Low | |
| Q9UHV2 | J11cd8 | CDK4-binding protein p34SEI1 | 144.2806 | 163 | 183.5211 | 163.6559 | 3.47E−01 | 1.77E−01 | 1 | Unchanged Low | |
| P02810 | F07gh6 | proline-rich protein HaeIII subfan | 410.3478 | 392 | 183.6977 | 328.6668 | −1.16E+00 | −6.62E−02 | 1 | Unchanged Low | |
| O94903 | K04cd7 | proline synthetase co-transcribed | 242.2062 | 146 | 183.9744 | 190.7733 | −3.97E−01 | −7.29E−01 | 1 | Unchanged Low | |
| Q9P0W6 | I03ef4 | lipopolysaccharide specific resp | 149.1418 | 177 | 184.0725 | 169.9232 | 3.04E−01 | 2.43E−01 | 1 | Unchanged Low | |
| Q9H8X4 | O15gh5 | stromal membrane-associated pr | 358.1542 | 342 | 184.0879 | 294.6026 | −9.60E−01 | −6.84E−02 | 1 | Unchanged Low | |
| P02786 | D24ef5 | transferrin receptor (p90: CD71) | 320.8365 | 257 | 184.2545 | 254.0696 | −8.00E−01 | −3.19E−01 | 1 | Unchanged Low | |
| | E15ef1 | immunoglobulin heavy constant | 386.489 | 176 | 184.3533 | 249.0303 | −1.07E+00 | −1.13E+00 | 1 | Unchanged Low | |
| Q99717 | J01ab7 | MAD: mothers against decapent | 227.5817 | 153 | 184.3978 | 188.4757 | −3.04E−01 | −5.69E−01 | 1 | Unchanged Low | |
| Q15036 | B23gh1 | sorting nexin 17 | 303.8909 | 411 | 184.4831 | 299.6453 | −7.20E−01 | 4.34E−01 | 1 | Unchanged Low | |
| P31751 | A08ab2 | v-akt murine thymoma viral onco | 253.7531 | 253 | 184.8245 | 230.4512 | −4.57E−01 | −5.57E−03 | 1 | Unchanged Low | |
| P11177 | G21ab8 | pyruvate dehydrogenase (lipoam | 229.1486 | 150 | 185.0719 | 188.1769 | −3.08E−01 | −6.08E−01 | 1 | Unchanged Low | |
| Q9UEQ6 | F22ab2 | arrestin; beta 2 | 190.7331 | 128 | 185.1221 | 167.9086 | −4.31E−02 | −5.77E−01 | 1 | Unchanged Low | |
| Q16531 | J11ab5 | damage-specific DNA binding pr | 205.1934 | 229 | 185.1363 | 206.5357 | −1.48E−01 | 1.60E−01 | 1 | Unchanged Low | |
| P28698 | G24cd3 | zinc finger protein 42 (myeloid-sp | 76.12612 | 105 | 185.3347 | 122.3029 | 1.28E+00 | 4.70E−01 | 1 | Unchanged Low | |
| P41968 | N12ef4 | melanocortin 3 receptor | 225.3987 | 287 | 185.5363 | 232.6783 | −2.81E−01 | 3.49E−01 | 1 | Unchanged Low | |
| P54920 | E03cd4 | N-ethylmaleimide-sensitive facto | 306.753 | 267 | 185.697 | 252.9933 | −7.24E−01 | −2.03E−01 | 1 | Unchanged Low | |
| Q99487 | C19ab8 | platelet-activating factor acetylhy | 228.4138 | 241 | 185.7091 | 218.5222 | −2.99E−01 | 8.00E−02 | 1 | Unchanged Low | |
| Q9H0C8 | N15gh7 | integrin-linked kinase-associated | 120.8459 | 109 | 185.7532 | 138.517 | 6.20E−01 | −1.49E−01 | 1 | Unchanged Low | |
| P17252 | F17ab8 | protein kinase C: alpha | 108.3851 | 74 | 185.8466 | 122.7553 | 7.78E−01 | −5.50E−01 | 1 | Unchanged Low | |
| P06493 | B01ef5 | cell division cycle 2; G1 to S and | 108.1696 | 76.2 | 185.8754 | 123.4207 | 7.81E−01 | −5.05E−01 | 1 | Unchanged Low | |
| O00139 | C14ab7 | kinesin heavy chain member 2 | 140.4141 | 205 | 185.9437 | 177.1988 | 4.05E−01 | 5.48E−01 | 1 | Unchanged Low | |
| P54259 | N05ef6 | dentatorubral-pallidoluysian atro | 165.5295 | 152 | 186.0962 | 167.773 | 1.69E−01 | −1.26E−01 | 1 | Unchanged Low | |
| Q99757 | G10cd8 | thioredoxin 2 | 309.0415 | 163 | 186.106 | 219.4587 | −7.32E−01 | −9.21E−01 | 1 | Unchanged Low | |
| Q9NW68 | B12gh2 | hypothetical protein FLJ10276 | 107.4044 | 242 | 186.1575 | 178.6551 | 7.93E−01 | 1.17E+00 | 1 | Unchanged Low | |
| P16106 | O16gh6 | H3 histone family: member J | 137.9914 | 135 | 186.2589 | 152.9203 | 4.33E−01 | −3.69E−02 | 1 | Unchanged Low | |
| P40692 | M06ef6 | mutL homolog 1; colon cancer: n | 98.77988 | 108 | 186.2872 | 131.0441 | 9.15E−01 | 1.30E−01 | 1 | Unchanged Low | |
| Q99798 | E01ab2 | aconitase 2; mitochondrial | 615.211 | 291 | 186.5524 | 364.1757 | −1.72E+00 | −1.08E+00 | 1 | Unchanged Low | |
| P53618 | A11ef3 | coatomer protein complex; subur | 238.4951 | 102 | 188.6268 | 175.716 | −3.54E−01 | −1.23E+00 | 1 | Unchanged Low | |
| Q9UJ69 | N24gh2 | Toll-interacting protein | 211.4055 | 259 | 186.6408 | 219.0188 | −1.80E−01 | 2.93E−01 | 1 | Unchanged Low | |
| Q9NRM2 | P22ef8 | zinc finger protein 277 | 232.9567 | 227 | 186.8854 | 215.6334 | −3.18E−01 | −3.70E−02 | 1 | Unchanged Low | |
| Q92879 | B06cd6 | CUG triplet repeat; RNA binding | 217.3752 | 198 | 187.122 | 200.6744 | −2.16E−01 | −1.38E−01 | 1 | Unchanged Low | |
| O75399 | H22gh1 | deformed epidermal autoregulat | 189.8387 | 266 | 187.2872 | 214.4669 | −1.95E−02 | 4.88E−01 | 1 | Unchanged Low | |
| Q9BZE4 | I17cd8 | G protein-binding protein CRFG | 151.2547 | 214 | 187.3094 | 184.3092 | 3.08E−01 | 5.03E−01 | 1 | Unchanged Low | |
| Q9NXB2 | G08gh2 | hypothetical protein FLJ20343 | 259.0672 | 285 | 187.4246 | 243.8822 | −4.67E−01 | 1.38E−01 | 1 | Unchanged Low | |
| O95424 | M06ef4 | MYLE protein | 103.2077 | 184 | 187.5574 | 158.2861 | 8.62E−01 | 8.35E−01 | 1 | Unchanged Low | |
| P54577 | D14cd3 | tyrosyl-tRNA synthetase | 145.5748 | 108 | 187.5756 | 146.9084 | 3.66E−01 | −4.36E−01 | 1 | Unchanged Low | |
| O43692 | N03ef7 | protease inhibitor 15 | 102.5299 | 163 | 187.7565 | 150.9471 | 8.73E−01 | 6.65E−01 | 1 | Unchanged Low | |
| O60507 | J03cd3 | tyrosylprotein sulfotransferase 1 | 190.271 | 136 | 188.0843 | 171.4862 | −1.67E−02 | −4.83E−01 | 1 | Unchanged Low | |
| Q9BYS9 | B15gh5 | MMS19-like (MET18 homolog; S. | 99.29429 | 117 | 188.1006 | 134.7877 | 9.22E−01 | 2.36E−01 | 1 | Unchanged Low | |
| P32780 | G11ab6 | general transcription factor IIH; p | 258.9396 | 180 | 188.3175 | 209.0492 | −4.59E−01 | −5.25E−01 | 1 | Unchanged Low | |
| Q14004 | D07ef5 | cell division cycle 2-like 5 (cholin | 92.56943 | 158 | 188.3656 | 146.1954 | 1.02E+00 | 7.68E−01 | 1 | Unchanged Low | |
| Q15910 | L08ab5 | enhancer of zeste homolog 2 (Dr | 213.1801 | 306 | 188.8314 | 235.9636 | −1.75E−01 | 5.21E−01 | 1 | Unchanged Low | |
| P47974 | H22ab4 | zinc finger protein 36; C3H type-I | 187.7102 | 163 | 188.8801 | 179.8162 | 8.96E−03 | −2.05E−01 | 1 | Unchanged Low | |
| Q13177 | E20ab8 | p21 (CDKN1A)-activated kinase: | 367.7706 | 232 | 188.9048 | 262.9597 | −9.61E−01 | −6.63E−01 | 1 | Unchanged Low | |
| | P16gh8 | baculoviral IAP repeat-containg | 251.3679 | 126 | 189.0052 | 188.6409 | −4.11E−01 | −1.00E+00 | 1 | Unchanged Low | |
| P98172 | J01ef7 | ephrin-B1 | 122.8828 | 109 | 189.5198 | 140.4039 | 6.25E−01 | −1.75E−01 | 1 | Unchanged Low | |
| Q9BQB1 | N04gh6 | hypothetical protein FLJ10342 | 113.906 | 172 | 189.7648 | 158.4309 | 7.36E−01 | 5.91E−01 | 1 | Unchanged Low | |
| O75472 | H11cd5 | DnaJ (Hsp40) homolog; subfamil | 227.1041 | 204 | 189.9354 | 206.8756 | −2.58E−01 | −1.58E−01 | 1 | Unchanged Low | |
| O75608 | K06cd6 | lysophospholipase I | 357.6112 | 374 | 190.1833 | 307.2824 | −9.11E−01 | 6.48E−02 | 1 | Unchanged Low | |
| O00193 | E14ef3 | small acidic protein | 223.9793 | 284 | 190.2397 | 232.7223 | −2.36E−01 | 3.42E−01 | 1 | Unchanged Low | |
| Q14452 | B08ab5 | glucocorticoid receptor DNA biN | 156.8451 | 147 | 190.439 | 164.6255 | 2.80E−01 | −9.75E−02 | 1 | Unchanged Low | |
| P55268 | P18ef6 | taminin; beta 2 (laminin S) | 125.0717 | 152 | 190.5787 | 155.8763 | 6.08E−01 | 2.81E−01 | 1 | Unchanged Low | |
| Q9P006 | A20ef8 | mitochondrial ribosomal protein | 114.6952 | 151 | 190.6578 | 152.156 | 7.33E−01 | 3.98E−01 | 1 | Unchanged Low | |
| Q9Y3B5 | P07ef1 | CGI-111 protein | 158.6983 | 176 | 190.6804 | 175.2567 | 2.65E−01 | 1.52E−01 | 1 | Unchanged Low | |
| Q9NQ34 | A10gh4 | chromosome 11 open reading fra | 144.3438 | 161 | 190.6944 | 165.3815 | 4.02E−01 | 1.59E−01 | 1 | Unchanged Low | |
| Q9NWY4 | O14gh2 | hypothetical protein FLJ20534 | 70.02507 | 110 | 191.0561 | 123.5579 | 1.45E+00 | 6.46E−01 | 1 | Unchanged Low | |
| Q9UBS8 | P17ab2 | ring finger protein 14 | 227.9827 | 247 | 191.1542 | 221.9109 | −2.54E−01 | 1.13E−01 | 1 | Unchanged Low | |
| Q9UBS4 | J06ef2 | DnaJ (Hsp40) homolog; subfamil | 181.8446 | 175 | 191.2168 | 182.7428 | 7.25E−02 | −5.40E−02 | 1 | Unchanged Low | |
| Q12797 | J22ab2 | aspartate beta-hydroxylase | 101.2251 | 86.5 | 191.3988 | 126.3618 | 9.19E−01 | −2.27E−01 | 1 | Unchanged Low | |
| Q9H4I9 | P10gh6 | hypothetical gene supported by | 167.5049 | 219 | 191.4831 | 192.7801 | 1.93E−01 | 3.89E−01 | 1 | Unchanged Low | |
| O14907 | E24ef8 | Tax interaction protein 1 | 351.7729 | 403 | 191.7684 | 315.6313 | −8.75E−01 | 1.97E−01 | 1 | Unchanged Low | |
| Q14673 | B09gh1 | Bcl-2-associated transcription fa | 97.16912 | 117 | 191.9499 | 135.533 | 9.82E−01 | 2.74E−01 | 1 | Unchanged Low | |
| Q9NWB7 | P01gh2 | estrogen-related receptor beta lik | 567.7076 | 84.6 | 192.0982 | 281.455 | −1.56E+00 | −2.75E+00 | 1 | Unchanged Low | |
| Q9BVH4 | G21gh6 | hypothetical protein MGC3196 | 164.8572 | 169 | 192.4133 | 175.4765 | 2.23E−01 | 3.72E−02 | 1 | Unchanged Low | |
| P22897 | I17ab7 | mannose receptor; C type 1 | 77.30638 | 100 | 192.4612 | 123.4215 | 1.32E+00 | 3.78E−01 | 1 | Unchanged Low | |
| P19012 | L04ab6 | keratin 15 | 146.5632 | 130 | 192.6097 | 156.5003 | 3.94E−01 | −1.69E−01 | 1 | Unchanged Low | |
| Q14738 | C16cd1 | protein phosphatase 2; regulator | 224.4642 | 183 | 192.7984 | 200.124 | −2.19E−01 | −2.94E−01 | 1 | Unchanged Low | |
| O95152 | F04cd4 | vesicle trafficking protein | 197.6319 | 196 | 192.9314 | 195.3686 | −3.47E−02 | −1.53E−02 | 1 | Unchanged Low | |
| P78426 | A09cd3 | NK6 transcription factor homolog | 198.5291 | 353 | 193.0203 | 248.2544 | −4.06E−02 | 8.31E−01 | 1 | Unchanged Low | |
| Q14790 | P14ab3 | caspase 8: apoptosis-related cys | 124.8603 | 284 | 193.1013 | 200.6718 | 6.29E−01 | 1.19E+00 | 1 | Unchanged Low | |
| P07711 | N13ab4 | cathepsin L | 137.16 | 121 | 193.1454 | 150.5861 | 4.94E−01 | −1.75E−01 | 1 | Unchanged Low | |
| P32942 | B02ab6 | intercellular adhesion molecule 3 | 140.3673 | 140 | 193.1656 | 157.6897 | 4.61E−01 | −8.57E−03 | 1 | Unchanged Low | |
| | L18ab2 | ATPase: Na+/K+ transporting: al | 188.9371 | 131 | 194.2503 | 171.3764 | 4.00E−02 | −5.29E−01 | 1 | Unchanged Low | |
| Q9NWS0 | F13gh2 | hypothetical protein FLJ20643 | 81.29108 | 107 | 194.4652 | 127.6547 | 1.26E+00 | 3.99E−01 | 1 | Unchanged Low | |
| Q9NWZ5 | O06gh2 | uridine kinase-like 1 | 176.188 | 358 | 194.4782 | 242.9739 | 1.42E−01 | 1.02E+00 | 1 | Unchanged Low | |
| O75792 | D15cd6 | ribonuclease HI: large subunit | 100.3515 | 99.6 | 194.705 | 131.5441 | 9.56E−01 | −1.12E−02 | 1 | Unchanged Low | |
| P05141 | I04ab2 | solute carrier family 25 (mitochor | 454.9832 | 311 | 194.7577 | 320.3918 | −1.22E+00 | −5.47E−01 | 1 | Unchanged Low | |
| Q9NX38 | K22gh2 | hypothetical protein FLJ20457 | 140.7383 | 161 | 195.2668 | 165.7012 | 4.72E−01 | 1.95E−01 | 1 | Unchanged Low | |
| Q9Y682 | A18cd8 | sec22 homolog | 193.7656 | 271 | 195.3918 | 220.1941 | 1.21E−02 | 4.86E−01 | 1 | Unchanged Low | |
| P78381 | C02cd4 | solute carrier family 35 (UDP-gal | 276.1892 | 207 | 195.6585 | 226.4009 | −4.97E−01 | −4.14E−01 | 1 | Unchanged Low | |
| Q9UMP5 | B23ef3 | replication initiation region protei | 270.4297 | 179 | 195.6957 | 214.9641 | −4.67E−01 | −5.97E−01 | 1 | Unchanged Low | |
| Q9P0Q0 | C15ef3 | hypothetical protein HSPC228 | 129.6054 | 241 | 195.7376 | 188.838 | 5.95E−01 | 8.96E−01 | 1 | Unchanged Low | |
| Q9UBQ3 | M12cd7 | polyamine-modulated factor 1 | 189.4397 | 172 | 195.7465 | 185.7473 | 4.72E−02 | −1.39E−01 | 1 | Unchanged Low | |
| P27540 | D22ab2 | aryl hydrocarbon receptor nuclea | 239.4814 | 258 | 195.776 | 231.071 | −2.91E−01 | 1.07E−01 | 1 | Unchanged Low | |
| Q9V2W1 | F03cd5 | thyroid hormone receptor-associa | 160.2008 | 248 | 195.7775 | 201.3236 | 2.89E−01 | 6.30E−01 | 1 | Unchanged Low | |
| P82267 | E15ab4 | adaptor-related protein complex | 198.6022 | 231 | 195.7793 | 208.571 | −2.07E−02 | 2.20E−01 | 1 | Unchanged Low | |
| P00918 | C16ab3 | carbonic anhydrase II | 283.4506 | 172 | 195.9099 | 216.9569 | −5.33E−01 | −7.25E−01 | 1 | Unchanged Low | |
| Q9Y663 | E07cd6 | heparan sulfate (glucosaniine) 3- | 259.052 | 99.8 | 196.1045 | 184.9847 | −4.02E−01 | −1.38E+00 | 1 | Unchanged Low | |
| Q9NX40 | K18gh2 | ovarian carcinoma immunoreacti | 119.6155 | 247 | 196.1836 | 187.7106 | 7.14E−01 | 1.05E+00 | 1 | Unchanged Low | |
| P50222 | L09ab7 | mesenchyme homeo box 2 (grow | 165.3048 | 223 | 196.5502 | 195.0639 | 2.50E−01 | 4.34E−01 | 1 | Unchanged Low | |
| Q13610 | C16cd7 | nuclear phosphoprotein similar t | 160.8948 | 331 | 196.7055 | 229.4375 | 2.90E−01 | 1.04E+00 | 1 | Unchanged Low | |
| P49591 | B12cd2 | seryl-tRNA synthetase | 152.5268 | 69.6 | 196.7944 | 139.6467 | 3.68E−01 | −1.13E+00 | 1 | Unchanged Low | |
| P04554 | C19gh5 | protamine 3 | 314.8934 | 296 | 196.9835 | 269.3189 | −6.77E−01 | −8.89E−02 | 1 | Unchanged Low | |
| P02538 | N12ef6 | keratin 6A | 227.8925 | 277 | 197.1759 | 233.9139 | −2.09E−01 | 2.80E−01 | 1 | Unchanged Low | |
| Q13637 | G23cd7 | RAB32: member RAS oncogene | 392.0158 | 269 | 197.2609 | 286.1382 | −9.91E−01 | −5.43E−01 | 1 | Unchanged Low | |
| P39086 | J02ab3 | glutamate receptor; ionotropic; k | 184.3318 | 210 | 197.334 | 197.2285 | 9.83E−02 | 1.88E−01 | 1 | Unchanged Low | |
| Q9H8F9 | H20gh5 | hypothetical protein 24432 | 287.08 | 202 | 197.402 | 228.8927 | −5.40E−01 | −5.06E−01 | 1 | Unchanged Low | |
| Q99460 | L13ab8 | proteasome (prosome; macropai | 298.3527 | 228 | 197.4025 | 241.22 | −5.96E−01 | −3.89E−01 | 1 | Unchanged Low | |
| Q9UKP6 | D20gh1 | G protein-coupled receptor 14 | 213.3717 | 142 | 197.5131 | 184.3366 | −1.11E−01 | −5.86E−01 | 1 | Unchanged Low | |
| Q16589 | A04ef5 | cyclin G2 | 99.61434 | 214 | 197.5658 | 170.3526 | 9.88E−01 | 1.10E+00 | 1 | Unchanged Low | |
| O95316 | M03cd5 | ribosomal protein S6 kinase; 90 k | 144.8234 | 145 | 197.5662 | 162.324 | 4.48E−01 | −2.40E−03 | 1 | Unchanged Low | |
| O14545 | A09cd7 | FLN29 gene product | 238.4292 | 288 | 197.8936 | 241.5146 | −2.69E−01 | 2.74E−01 | 1 | Unchanged Low | |
| Q9Y249 | P03cd6 | homologous to yeast nitrogen pe | 191.1146 | 224 | 198.1049 | 204.4863 | 5.18E−02 | 2.31E−01 | 1 | Unchanged Low | |
| Q92794 | K12cd4 | zinc finger protein 220 | 134.123 | 179 | 198.312 | 170.3523 | 5.64E−01 | 4.13E−01 | 1 UnchangedLow | | |
| P36894 | A12ab3 | bone morphogenetic protein rece | 149.2953 | 236 | 198.4963 | 194.5315 | 4.11E−01 | 6.59E−01 | 1 | Unchanged Low | |
| P79525 | L21ab7 | MHC class I polypeptide-related | 120.8289 | 125 | 198.5782 | 148.0291 | 7.17E−01 | 4.53E−02 | 1 | Unchanged Low | |
| O14947 | K04ab6 | laminin: beta 3 (nicein (125kD): | 360.8887 | 284 | 199.2636 | 281.2972 | −8.57E−01 | −3.47E−01 | 1 | Unchanged Low | |
| Q12899 | K16cd3 | tripartite motif-containing 26 | 188.6935 | 250 | 199.2998 | 212.5994 | 7.89E−02 | 4.05E−01 | 1 | Unchanged Low | |
| | M24gh8 | zinc finger protein 289: IDt regul | 91.37228 | 137 | 199.4969 | 142.4645 | 1.13E+00 | 5.79E−01 | 1 | Unchanged Low | |
| Q9UIJ5 | A18ef2 | zinc finger; DHHC domain contai | 436.5048 | 452 | 199.696 | 362.7132 | −1.13E+00 | 5.01E−02 | 1 | Unchanged Low | |
| P46821 | J11ab7 | microtubule-associated protein 1 | 121.2356 | 136 | 199.8286 | 152.4972 | 7.21E−01 | 1.70E−01 | 1 | Unchanged Low | |
| O60906 | P04cd1 | sphingomyelin phosphodiesteras | 282.5771 | 281 | 199.835 | 254.5351 | −5.00E−01 | −7.08E−03 | 1 | Unchanged Low | |
| P04040 | O18ab5 | catalase | 160.7098 | 239 | 199.9283 | 199.8034 | 3.15E−01 | 5.71E−01 | 1 | Unchanged Low | |
| O60709 | B16cd5 | dynamin 1-like | 85.33527 | 220 | 199.9492 | 168.4792 | 1.23E+00 | 1.37E+00 | 1 | Unchanged Low | |
| Q14714 | A02cd4 | sarcospan (Kras oncogene-asso | 378.724 | 148 | 200.1413 | 242.1486 | −9.20E−01 | −1.36E+00 | 1 | Unchanged Low | |
| P39687 | F01cd4 | acidic (leucine-rich) nuclear phos | 226.8272 | 108 | 200.186 | 178.3873 | −1.80E−01 | −1.07E+00 | 1 | Unchanged Low | |
| O75341 | K16cd4 | BRCA1 associated protein | 151.895 | 209 | 200.2666 | 187.1088 | 3.99E−01 | 4.62E−01 | 1 | Unchanged Low | |
| Q9NUN1 | D16gh3 | hypothetical protein FLJ11259 | 152.5767 | 196 | 200.5435 | 182.989 | 3.94E−01 | 3.60E−01 | 1 | Unchanged Low | |
| Q9UHR5 | L22ef7 | transcriptional regulator protein | 225.1172 | 345 | 200.7483 | 256.9591 | −1.65E−01 | 6.16E−01 | 1 | Unchanged Low | |
| Q9UMX3 | D15gh8 | BCL2-related ovarian killer | 185.2616 | 176 | 200.9857 | 187.4707 | 1.18E−01 | −7.26E−02 | 1 | Unchanged Low | |
| Q02297 | J05ef6 | neuregulin 1 | 89.51288 | 234 | 201.3025 | 175.0464 | 1.17E+00 | 1.39E+00 | 1 | Unchanged Low | |
| Q9BQ83 | A21gh6 | hypothetical protein MGC5178 | 302.7768 | 398 | 201.3498 | 300.7124 | −5.89E−01 | 3.95E−01 | 1 | Unchanged Low | |
| O43491 | C22ab4 | erythrocyte membrane protein ba | 265.5557 | 144 | 201.5206 | 203.6633 | −3.98E−01 | −8.84E−01 | 1 | Unchanged Low | |
| Q92922 | N16cd1 | SWI/SNF related; matrix associa | 251.3134 | 195 | 201.571 | 216.0309 | −3.18E−01 | −3.84E−01 | 1 | Unchanged Low | |
| Q16099 | J11ef3 | glutamate receptor: ionotropic: k | 200.0311 | 288 | 201.66 | 229.9201 | 1.17E−02 | 5.26E−01 | 1 | Unchanged Low | |
| O95328 | B06gh4 | hypothetical protein 628 | 253.2751 | 240 | 201.6989 | 231.7737 | −3.29E−01 | −7.56E−02 | 1 | Unchanged Low | |
| Q06265 | C15cd1 | polymyositis/scleroderma autoan | 167.3169 | 182 | 202.2097 | 183.9827 | 2.73E−01 | 1.25E−01 | 1 | Unchanged Low | |
| Q01780 | K12gh6 | polymyositis/scleroderma autoan | 196.9508 | 270 | 202.2308 | 223.1866 | 3.82E−02 | 4.57E−01 | 1 | Unchanged Low | |
| Q9NPF9 | G07ef3 | HeLa cyclin-dependent kinase 2 | 251.6953 | 170 | 202.5242 | 207.9337 | −3.14E−01 | −5.70E−01 | 1 | Unchanged Low | |
| Q9P1U0 | B03ef4 | zinc ribbon domain containing; 1 | 110.7474 | 151 | 202.7351 | 154.8483 | 8.72E−01 | 4.48E−01 | 1 | Unchanged Low | |
| Q13480 | C02ab5 | GRB2-associated binding protein | 171.5634 | 150 | 203.065 | 174.7367 | 2.43E−01 | −1.98E−01 | 1 | Unchanged Low | |
| O95205 | O05gh1 | C3H-type zinc finger protein; sim | 136.7838 | 267 | 203.3797 | 202.2261 | 5.12E−01 | 9.62E−01 | 1 | Unchanged Low | |
| O76091 | I11cd1 | nitrilase 1 | 194.24 | 179 | 203.4317 | 192.2638 | 6.67E−02 | −1.17E−01 | 1 | Unchanged Low | |
| Q9Y364 | F21ef2 | DKF7P434J154 protein | 208.15 | 322 | 203.5516 | 244.4561 | −3.22E−02 | 6.28E−01 | 1 | Unchanged Low | |
| P51116 | I14cd5 | fragile X mental retardation: auto | 215.9784 | 213 | 203.6388 | 210.8166 | −8.49E−02 | −2.12E−02 | 1 | Unchanged Low | |
| P49915 | H19cd4 | guanine monphosphate syntheta | 175.5001 | 115 | 203.6534 | 164.6237 | 2.15E−01 | −6.13E−01 | 1 | Unchanged Low | |
| Q9UHR3 | O04cd8 | nasopharyngeal carcinoma susc | 120.0173 | 196 | 203.8212 | 173.3257 | 7.64E−01 | 7.09E−01 | 1 | Unchanged Low | |
| Q15007 | O10cd5 | Wilms' tumour 1-associating prot | 237.1077 | 258 | 204.228 | 233.0967 | −2.15E−01 | 1.22E−01 | 1 | Unchanged Low | |
| P24941 | B03ef5 | cyclin-dependent kinase 2 | 98.60804 | 214 | 204.3554 | 172.2465 | 1.05E+00 | 1.12E+00 | 1 | Unchanged Low | |
| P53634 | L01ef7 | cathepsin C | 97.3314 | 77.9 | 204.771 | 126.6548 | 1.07E+00 | −3.22E−01 | 1 | Unchanged Low | |
| Q13123 | K03ef7 | IK cytokine; down-regulator of H | 108.6134 | 153 | 204.9565 | 155.5636 | 9.16E−01 | 4.95E−01 | 1 | Unchanged Low | |
| Q9NUT6 | P21gh3 | hypothetical protein FLJ11151 | 285.3106 | 565 | 205.0219 | 351.7208 | −4.77E−01 | 9.85E−01 | 1 | Unchanged Low | |
| Q9Y279 | B23cd7 | Ig superfamily protein | 356.8489 | 226 | 205.0366 | 262.6019 | −7.99E−01 | −6.59E−01 | 1 | Unchanged Low | |
| O75319 | H07cd3 | dual specificity phosphatase 11 ( | 250.2151 | 205 | 205.1456 | 220.2678 | −2.87E−01 | −2.84E−01 | 1 | Unchanged Low | |
| Q9NYH9 | L08ef3 | hepatocellular carcinoma-associ | 287.2586 | 283 | 205.223 | 258.6032 | −4.85E−01 | −1.99E−02 | 1 | Unchanged Low | |
| Q9UNF0 | M20cd7 | protein kinase C and casein kina | 438.5503 | 349 | 205.53 | 331.0599 | −1.09E+00 | −3.29E−01 | 1 | Unchanged Low | |
| O60437 | D03ab8 | periplakin | 443.9882 | 301 | 205.5879 | 316.8358 | −1.11E+00 | −5.61E−01 | 1 | Unchanged Low | |
| Q9NZN8 | N22cd8 | CCR4-NOT transcription comple: | 189.665 | 224 | 205.673 | 206.5153 | 1.17E−01 | 2.41E−01 | 1 | Unchanged Low | |
| Q14232 | A18ab4 | eukaryotic translation initiation fa | 273.7271 | 210 | 205.7436 | 229.9659 | −4.12E−01 | −3.79E−01 | 1 | Unchanged Low | |
| O00142 | C03ef5 | thymidine kinase 2; mitochondria | 124.7128 | 147 | 205.9951 | 159.1674 | 7.24E−01 | 2.35E−01 | 1 | Unchanged Low | |
| O60443 | N15ab5 | deafness; autosomal dominant 5 | 122.4035 | 264 | 206.3187 | 197.4327 | 7.53E−01 | 1.11E+00 | 1 | Unchanged Low | |
| O95406 | J20cd5 | comichon-like | 181.7181 | 138 | 206.4442 | 175.4375 | 1.84E−01 | −3.95E−01 | 1 | Unchanged Low | |
| Q12841 | E24cd7 | follistatin-like 1 | 80.93058 | 97.7 | 206.5097 | 128.3877 | 1.35E+00 | 2.72E−01 | 1 | Unchanged Low | |
| O15217 | B04ab5 | glutathione S-transferase A4 | 78.38055 | 91.3 | 206.6118 | 125.424 | 1.40E+00 | 2.20E−01 | 1 | Unchanged Low | |
| P10645 | E01ab4 | chromogranin A (parathyroid sec | 115.7467 | 283 | 206.8714 | 201.8922 | 8.38E−01 | 1.29E+00 | 1 | Unchanged Low | |
| Q9HA66 | D09gh5 | G protein beta subunit-like | 198.1992 | 192 | 206.9573 | 199.1923 | 6.24E−02 | −4.27E−02 | 1 | Unchanged Low | |
| Q9Y439 | B06ef8 | PRP31 pre-mRNA processing fa | 175.7234 | 310 | 207.0904 | 230.9065 | 2.37E−01 | 8.19E−01 | 1 | Unchanged Low | |
| Q9Y386 | G08ef2 | CGI-78 protein | 325.6509 | 248 | 207.7702 | 259.7061 | −6.48E−01 | −4.06E−01 | 1 | Unchanged Low | |
| P53990 | D21gh1 | KIAA0174 gene product | 230.1187 | 401 | 207.9831 | 279.676 | −1.46E−01 | 8.01E−01 | 1 | Unchanged Low | |
| O95865 | O11ef3 | dimethylarginine dimethylaminoh | 125.0105 | 157 | 208.0048 | 163.3476 | 7.35E−01 | 3.29E−01 | 1 | Unchanged Low | |
| | B21ef1 | immunoglobulin lambda-like poly | 142.7367 | 230 | 208.1338 | 193.5142 | 5.44E−01 | 6.86E−01 | 1 | Unchanged Low | |
| Q15834 | I17cd7 | hepatitis delta antigen-interacting | 320.7647 | 226 | 208.1753 | 251.6031 | −6.24E−01 | −5.06E−01 | 1 | Unchanged Low | |
| P18846 | H05ab4 | activating transcription factor 1 | 470.9776 | 206 | 208.3404 | 295.2222 | −1.18E+00 | −1.19E+00 | 1 | Unchanged Low | |
| Q14349 | A23cd7 | HLA-B associated transcript 8 | 138.8938 | 175 | 205.5984 | 174.3142 | 5.87E−01 | 3.37E−01 | 1 | Unchanged Low | |
| Q9NVM5 | M08gh3 | hypothetical protein FLJ10637 | 98.19923 | 115 | 209.0315 | 140.794 | 1.09E+00 | 2.30E−01 | 1 | Unchanged Low | |
| P12955 | N18ab7 | peptidase D | 133.8157 | 192 | 209.0325 | 178.2362 | 6.43E−01 | 5.20E−01 | 1 | Unchanged Low | |
| Q99700 | D14cd1 | spinocerebellar ataxia 2 (olivopo | 191.0044 | 240 | 209.0555 | 213.1956 | 1.30E−01 | 3.27E−01 | 1 | Unchanged Low | |
| 09UJA2 | E21gh3 | chromosome 20 open reading fra | 134.2553 | 316 | 209.227 | 219.8275 | 6.40E−01 | 1.23E+00 | 1 | Unchanged Low | |
| Q02246 | J21ef6 | contactin 2 (axonal) | 108.0043 | 68.8 | 209.3915 | 128.7469 | 9.55E−01 | −6.50E−01 | 1 | Unchanged Low | |
| P48553 | E06cd2 | transmembrane protein 1 | 148.0375 | 238 | 209.4168 | 198.4177 | 5.00E−01 | 6.84E−01 | 1 | Unchanged Low | |
| Q99639 | K03cd2 | suppressor of Ty 5 homolog (S. c | 225.1962 | 296 | 209.795 | 243.5051 | −1.02E−01 | 3.92E−01 | 1 | Unchanged Low | |
| Q9NQX5 | N02gh4 | neural proliferation; differentiatio | 188.0695 | 206 | 209.8245 | 201.225 | 1.58E−01 | 1.30E−01 | 1 | Unchanged Low | |
| Q99799 | O08cd6 | nuclear RNA esport factor 1 | 205.4132 | 175 | 209.9237 | 196.7655 | 3.13E−02 | −2.32E−01 | 1 | Unchanged Low | |
| Q9BZV1 | J05gh7 | UBX domain-containing 1 | 127.7069 | 236 | 209.9733 | 191.2074 | 7.17E−01 | 8.86E−01 | 1 | Unchanged Low | |
| Q03113 | M19ab5 | guanine nucleotide binding prote | 140.2412 | 112 | 209.9975 | 154.1344 | 5.82E−01 | −3.22E−01 | 1 | Unchanged Low | |
| P01130 | B21ab6 | low density lipoprotein receptor ( | 301.1179 | 394 | 210.369 | 301.7776 | −5.17E−01 | 3.87E−01 | 1 | Unchanged Low | |
| Q9UHN6 | L11cd8 | transmembrane protein 2 | 182.3012 | 130 | 210.5088 | 174.2344 | 2.08E−01 | −4.89E−01 | 1 | Unchanged Low | |
| Q99738 | A03cd3 | pinin; desmosome associated pro | 200.7164 | 204 | 210.6944 | 205.0214 | 7.00E−02 | 2.10E−02 | 1 | Unchanged Low | |
| Q9Y576 | N03ef2 | ankyrin repeat and SOCS box-cc | 121.1139 | 76.2 | 210.8606 | 136.0527 | 8.00E−01 | −6.69E−01 | 1 | Unchanged Low | |
| P02304 | B03gh6 | H4 histone; family 2 | 81.15302 | 191 | 210.8651 | 161.1406 | 1.38E+00 | 1.24E+00 | 1 | Unchanged Low | |
| P01118 | I05ab7 | v-Ki-ras2 Kirsten rat sarcoma 2 v | 209.4177 | 239 | 211.0234 | 219.9522 | 1.10E−02 | 1.93E−01 | 1 | Unchanged Low | |
| P15924 | K13ef7 | desmoplakin (DPI; DPII) | 249.9083 | 263 | 211.0334 | 241.3819 | −2.44E−01 | 7.48E−02 | 1 | Unchanged Low | |
| O43597 | A19cd6 | sprouty homolog 2 (Drosophila) | 119.5051 | 155 | 211.0573 | 161.8827 | 8.21E−01 | 3.75E−01 | 1 | Unchanged Low | |
| P09622 | I06ab3 | dihydrolipoamide dehydrogenase | 346.3029 | 232 | 211.1679 | 263.2128 | −7.14E−01 | −5.77E−01 | 1 | Unchanged Low | |
| Q08257 | G16ef7 | crystallin; zeta (quinone reductas | 514.6998 | 301 | 211.6448 | 342.4839 | −1.28E+00 | −7.73E−01 | 1 | Unchanged Low | |
| Q9UHY8 | J09cd7 | fasciculation and elongation prot | 86.8735 | 95.1 | 211.8371 | 131.2705 | 1.29E+00 | 1.31E−01 | 1 | Unchanged Low | |
| P10646 | P23cd2 | tissue factor pathway inhibitor (lip | 596.8869 | 458 | 212.0146 | 422.403 | −1.49E+00 | −3.51E−01 | 1 | Unchanged Low | |
| Q92685 | L08cd5 | Not56 (D. melanogaster)-like pro | 195.2017 | 217 | 212.2397 | 208.1394 | 1.21E−01 | 1.53E−01 | 1 | Unchanged Low | |
| P34949 | I13ab7 | mannose phosphate isomerase | 102.2251 | 135 | 212.3424 | 149.8251 | 1.05E+00 | 4.00E−01 | 1 | Unchanged Low | |
| Q9NPE9 | M21ef8 | HSPC055 protein | 91.56792 | 157 | 212.3544 | 153.7649 | 1.21E+00 | 7.81E−01 | 1 | Unchanged Low | |
| Q99853 | J24ef7 | forkhead box B1 | 141.4272 | 105 | 212.4773 | 153.0918 | 5.87E−01 | −4.25E−01 | 1 | Unchanged Low | |
| Q9HD41 | C24gh4 | CHMP1.5 protein | 280.2988 | 230 | 212.8608 | 241.047 | −3.97E−01 | −2.85E−01 | 1 | Unchanged Low | |
| Q16625 | A10ab8 | occludin | 859.5688 | 338 | 212.9222 | 470.2634 | −2.01E+00 | −1.35E+00 | 1 | Unchanged Low | |
| P45973 | L18ef6 | chromobox homolog 5 (HP1 alph | 164.9514 | 124 | 213.1315 | 167.3186 | 3.70E−01 | −4.13E−01 | 1 | Unchanged Low | |
| Q9NVI9 | O12gh3 | hypothetical protein FLJ10707 | 136.0971 | 136 | 213.1484 | 161.8885 | 6.47E−01 | 3.42E−03 | 1 | Unchanged Low | |
| P37173 | O09ef7 | transforming growth factor: beta | 112.4415 | 208 | 213.3211 | 178.0564 | 9.24E−01 | 8.90E−01 | 1 | Unchanged Low | |
| Q9UKF7 | O21cd8 | retinal degeneration B beta | 333.6061 | 287 | 213.5389 | 277.9863 | −6.44E−01 | −2.18E−01 | 1 | Unchanged Low | |
| P16870 | J23ef7 | carboxypeptidase E | 81.85341 | 137 | 213.5973 | 144.0377 | 1.38E+00 | 7.40E−01 | 1 | Unchanged Low | |
| P00374 | D16ab3 | dihydrofolate reductase | 148.8142 | 59.6 | 213.6217 | 140.6638 | 5.22E−01 | −1.32E+00 | 1 | Unchanged Low | |
| P19957 | M04ab8 | protease inhibitor 3; skin-derived | 202.9357 | 582 | 213.9068 | 333.0661 | 7.60E−02 | 1.52E+00 | 1 | Unchanged Low | |
| O75962 | J24cd2 | triple functional domain (PTPRF | 233.7849 | 197 | 213.9409 | 215.0154 | −1.28E−01 | −2.45E−01 | 1 | Unchanged Low | |
| P16050 | C24ab2 | arachidonate 15-lipoxygenase | 288.5504 | 87.3 | 213.9824 | 196.6194 | −4.31E−01 | −1.72E+00 | 1 | Unchanged Low | |
| Q9Y3D2 | N14cd7 | pilin-like transcription factor | 200.9397 | 224 | 214.0069 | 213.0068 | 9.09E−02 | 1.57E−01 | 1 | Unchanged Low | |
| O00505 | J16ab6 | karyopherin alpha 3 (importin alp | 358.8945 | 313 | 214.0819 | 295.4165 | −7.45E−01 | −1.96E−01 | 1 | Unchanged Low | |
| Q9N7D8 | N15ef1 | acid claster protein 33 | 222.816 | 223 | 214.2958 | 220.0079 | −5.62E−02 | 6.21E−04 | 1 | Unchanged Low | |
| P46937 | G10cd6 | Yes-associated protein 1: 65kD | 219.0427 | 268 | 214.3846 | 233.924 | −3.10E−02 | 2.93E−01 | 1 | Unchanged Low | |
| P53384 | O05ab1 | nucleotide binding protein 1 (Min | 272.4995 | 223 | 214.5051 | 236.5867 | −3.45E−01 | −2.91E−01 | 1 | Unchanged Low | |
| Q9UI28 | K10ef4 | AD-003 protein | 120.4834 | 147 | 214.7067 | 160.6424 | 8.34E−01 | 2.84E−01 | 1 | Unchanged Low | |
| Q14651 | O20ab8 | plaslin 1 (I isoform) | 517.7448 | 303 | 214.7993 | 345.2493 | −1.27E+00 | −7.72E−01 | 1 | Unchanged Low | |
| P10914 | K01ef6 | interferon regulatory factor 1 | 310.699 | 395 | 214.9514 | 306.8604 | −5.32E−01 | 3.46E−01 | 1 | Unchanged Low | |
| P21281 | A03ab3 | ATPase; H+ transporting; lysoso | 299.07 | 638 | 215.0012 | 383.914 | −4.76E−01 | 1.09E+00 | 1 | Unchanged Low | |
| P53701 | G19ab6 | holocytochrome c synthase (cyto | 251.8825 | 415 | 215.1177 | 293.8906 | −2.28E−01 | 7.19E−01 | 1 | Unchanged Low | |
| Q9Y2B1 | C18ef3 | transmembrane protein 5 | 103.4737 | 123 | 21S.2492 | 147.2535 | 1.06E+00 | 2.50E−01 | 1 | Unchanged Low | |
| Q14166 | D23ef8 | KIAA0153 protein | 332.5834 | 276 | 215.4465 | 274.7657 | −6.26E−01 | −2.68E−01 | 1 | Unchanged Low | |
| Q15738 | B09ef4 | NAD(P) dependent steroid dehy | 253.061 | 171 | 215.5558 | 213.0647 | −2.31E−01 | −5.69E−01 | 1 | Unchanged Low | |
| Q16586 | I06cd1 | sarcoglycan; alpha (50kD dystro | 358.221 | 129 | 215.8058 | 234.3861 | −7.31E−01 | −1.47E+00 | 1 | Unchanged Low | |
| Q9Y6J8 | L05ef2 | map kinase phosphatase-like pr | 275.0054 | 326 | 215.9987 | 272.4953 | −3.48E−01 | 2.48E−01 | 1 | Unchanged Low | |
| Q9H5F2 | P17gh5 | hypothetical protein FLJ23499 | 204.1242 | 332 | 216.1176 | 250.8425 | 8.24E−02 | 7.03E−01 | 1 | Unchanged Low | |
| Q05655 | E02cd1 | protein kinase C: delta | 419.7056 | 297 | 216.1184 | 310.8344 | −9.58E−01 | −5.00E−01 | 1 | Unchanged Low | |
| Q9NXU7 | M03gh3 | hypothetical protein FLJ20048 | 240.4802 | 235 | 216.1338 | 230.6008 | −1.54E−01 | −3.21E−02 | 1 | Unchanged Low | |
| O15498 | P21cd6 | SNARE protein Ykt6 | 188.9561 | 242 | 216.7243 | 215.7355 | 1.98E−01 | 3.54E−01 | 1 | Unchanged Low | |
| Q92664 | F07ab4 | general transcription factor IIIA | 318.1862 | 193 | 216.7575 | 242.5629 | −5.54E−01 | −7.23E−01 | 1 | Unchanged Low | |
| Q9Y223 | N03cd5 | UDP-N-acetylglucosamine-2-epi | 207.311 | 295 | 217.1156 | 239.9617 | 6.67E−02 | 5.11E−01 | 1 | Unchanged Low | |
| O95816 | E21ab3 | BCL2-associated athanogene 2 | 84.7224 | 94.2 | 217.4527 | 132.1461 | 1.36E+00 | 1.53E−01 | 1 | Unchanged Low | |
| Q9H0K5 | A21gh8 | hypothetical protein DKFZp434H | 193.1934 | 324 | 217.6924 | 244.8706 | 1.72E−01 | 7.45E−01 | 1 | Unchanged Low | |
| 09BTJ1 | C03ef2 | ribosomal protein S27-like | 132.1497 | 411 | 218.2702 | 253.7125 | 7.24E−01 | 1.64E+00 | 1 | Unchanged Low | |
| Q9NRR5 | J23gh4 | chromosome 1 open reading fra | 138.2192 | 201 | 218.4149 | 185.8791 | 6.60E−01 | 5.40E−01 | 1 | Unchanged Low | |
| Q9Y5L3 | A13ab4 | ectonucleoside triphosphate diph | 235.2745 | 316 | 218.4547 | 256.4569 | −1.07E−01 | 4.24E−01 | 1 | Unchanged Low | |
| O75351 | K06cd5 | suppressor of K+ transport defec | 274.5936 | 311 | 218.6389 | 267.9692 | −3.29E−01 | 1.78E−01 | 1 | Unchanged Low | |
| O75443 | F07cd2 | tectorin alpha | 183.4591 | 150 | 218.7282 | 184.2202 | 2.54E−01 | −2.86E−01 | 1 | Unchanged Low | |
| Q9H7X4 | K04gh7 | KIAA1085 protein | 268.2576 | 372 | 218.7527 | 286.2489 | −2.94E−01 | 4.71E−01 | 1 | Unchanged Low | |
| O00255 | K22ab6 | multiple endocrine neoplasia I | 134.7209 | 117 | 218.9335 | 156.9374 | 7.01E−01 | −2.02E−01 | 1 | Unchanged Low | |
| Q9BV94 | D05gh3 | chromosome 20 open reading fra | 319.259 | 316 | 219.2324 | 284.912 | −5.42E−01 | −1.37E−02 | 1 | Unchanged Low | |
| O14813 | D10ab2 | aristaless homeobox (Drosophila | 226.3067 | 320 | 219.2567 | 255.131 | −4.57E−02 | 4.99E−01 | 1 | Unchanged Low | |
| P35269 | D15ab4 | general transcription factor IIF: p | 243.8395 | 213 | 219.8975 | 225.4377 | −1.49E−01 | −1.98E−01 | 1 | Unchanged Low | |
| Q12891 | E08ef5 | hyaluronoglucosaminidase 2 | 331.3833 | 311 | 220.4462 | 287.6363 | −5.88E−01 | −9.12E−02 | 1 | Unchanged Low | |
| O15162 | L08gh1 | phospholipid scramblase 1 | 440.6448 | 597 | 220.6688 | 419.5068 | −9.98E−01 | 4.39E−01 | 1 | Unchanged Low | |
| Q14469 | O08ab7 | hairy homolog (Drosophila) | 151.2148 | 251 | 221.0242 | 207.739 | 5.48E−01 | 7.31E−01 | 1 | Unchanged Low | |
| P06241 | P13ab4 | FYN oncogene related to SRC: F | 106.4214 | 82.5 | 221.2951 | 136.7385 | 1.06E+00 | −3.67E−01 | 1 | Unchanged Low | |
| Q9BVG9 | P17gh7 | phosphatidylserine synthase 2 | 225.5812 | 200 | 221.5588 | 215.8454 | −2.60E−02 | −1.71E−01 | 1 | Unchanged Low | |
| Q9P0P9 | K12ef2 | hypothetical protein LOC51249 | 290.4444 | 434 | 222.6323 | 315.5648 | −3.84E−01 | 5.78E−01 | 1 | Unchanged Low | |
| Q9UMF9 | L14cd3 | eukaryotic translation initiation fa | 207.6515 | 242 | 222.6344 | 224.2505 | 1.01E−01 | 2.24E−01 | 1 | Unchanged Low | |
| Q03527 | L07ab8 | proteasome (prosome; macropai | 441.6371 | 284 | 222.6794 | 316.1666 | −9.88E−01 | −6.36E−01 | 1 | Unchanged Low | |
| P31153 | J15ab7 | methionine adenosyltransferase | 245.2111 | 134 | 222.7802 | 200.5541 | −1.38E−01 | −8.75E−01 | 1 | Unchanged Low | |
| Q04741 | D21ef1 | empty spiracles homolog 1 (Dros | 240.3769 | 189 | 222.791 | 217.4148 | −1.10E−01 | −3.46E−01 | 1 | Unchanged Low | |
| Q13153 | L22ef5 | p21/Cdc42/Rac1-activated kinas | 243.198 | 277 | 222.8583 | 247.6258 | −1.26E−01 | 1.87E−01 | 1 | Unchanged Low | |
| Q9Y388 | H15ef2 | CGI-79 protein | 100.7214 | 181 | 223.2467 | 168.2598 | 1.15E+00 | 8.44E−01 | 1 | Unchanged Low | |
| Q9HC14 | K17gh5 | mitochondrial ribosomal protein S | 158.7783 | 200 | 223.2594 | 193.9429 | 4.92E−01 | 3.31E−01 | 1 | Unchanged Low | |
| P30281 | O08ef5 | cyclin D3 | 107.4859 | 167 | 223.2751 | 166.0499 | 1.05E+00 | 6.39E−01 | 1 | Unchanged Low | |
| Q9UFK2 | P05ef8 | DKFZP564K1964 protein | 133.0476 | 210 | 223.4229 | 188.8934 | 7.48E−01 | 6.60E−01 | 1 | Unchanged Low | |
| Q13011 | O23ab4 | enoyl Coenzyme A hydratase 1; | 337.9818 | 273 | 223.7187 | 278.2904 | −5.95E−01 | −3.07E−01 | 1 | Unchanged Low | |
| P09601 | E20ef7 | heme oxygenase (decycling) 1 | 186.4519 | 425 | 223.7447 | 278.3661 | 2.63E−01 | 1.19E+00 | 1 | Unchanged Low | |
| Q9BVZ8 | P23gh8 | hypothetical protein FLJ14936 | 126.689 | 127 | 224.0692 | 159.346 | 8.23E−01 | 6.71E−03 | 1 | Unchanged Low | |
| Q13889 | A11ab5 | general transcription factor IIH; p | 112.1935 | 202 | 224.1653 | 179.5227 | 9.99E−01 | 8.50E−01 | 1 | Unchanged Low | |
| P49703 | P23ab2 | ADP-ribosylation factor 4-like | 125.6732 | 91.2 | 224.1896 | 147.0234 | 8.35E−01 | −4.62E−01 | 1 | Unchanged Low | |
| Q9GZU7 | G11gh4 | nuclear LIM interactor-interacting | 305.5546 | 150 | 224.2596 | 226.5474 | −4.46E−01 | −1.03E+00 | 1 | Unchanged Low | |
| P10253 | M22ab3 | glucosidase: alpha: acid (Pompe | 187.3998 | 156 | 224.2782 | 189.0733 | 2.59E−01 | −2.69E−01 | 1 | Unchanged Low | |
| Q9Y3V5 | B18ef8 | hypothetical protein DKFZP586F | 303.8477 | 426 | 224.4103 | 318.1897 | −4.37E−01 | 4.89E−01 | 1 | Unchanged Low | |
| O14773 | P17ef7 | ceroid-lipofuscinosis; neuronal 2: | 169.6468 | 214 | 224.4535 | 202.7117 | 4.04E−01 | 3.35E−01 | 1 | Unchanged Low | |
| P22061 | G13cd1 | protein-L-isoaspartate (D-asparta | 118.1783 | 163 | 224.5569 | 168.6387 | 9.26E−01 | 4.66E−01 | 1 | Unchanged Low | |
| P14868 | K21ab4 | aspartyl-tRNA synthetase | 117.1946 | 78.1 | 225.0996 | 140.1468 | 9.42E−01 | −5.85E−Ol | 1 | Unchanged Low | |
| Q14938 | D16ef6 | nuclear factor I/X (CCAAT-bindin | 94.58773 | 84.9 | 225.3458 | 134.9545 | 1.25E+00 | −1.55E−01 | 1 | Unchanged Low | |
| O75968 | P21ef3 | pannexin 1 | 215.9189 | 216 | 225.5368 | 219.072 | 6.29E−02 | −1.06E−03 | 1 | Unchanged Low | |
| Q9H779 | C15gh6 | hypothetical protein MGC2821 | 138.4597 | 313 | 225.8086 | 225.7323 | 7.06E−01 | 1.18E+00 | 1 | Unchanged Low | |
| Q9BX72 | C02gh8 | IFP38 | 168.9682 | 172 | 225.8249 | 188.9468 | 4.18E−01 | 2.61E−02 | 1 | Unchanged Low | |
| O43563 | G24cd1 | solute carrier family 22 (organic | 133.7352 | 149 | 225.8428 | 169.4168 | 7.56E−01 | 1.53E−01 | 1 | Unchanged Low | |
| O15142 | D17ab2 | ARP2 actin-related protein 2 ho | 250.3086 | 215 | 226.0552 | 230.3093 | −1.47E−01 | −2.22E−01 | 1 | Unchanged Low | |
| Q9UJ41 | K17ef4 | putative Rab5 GDP/GTP exchang | 121.9096 | 165 | 226.5205 | 171.2118 | 8.94E−01 | 4.38E−01 | 1 | Unchanged Low | |
| P05231 | F21ef7 | interleukin 6 (interferon: beta 2) | 240.2349 | 673 | 226.529 | 379.972 | −8.48E−02 | 1.49E−00 | 1 | Unchanged Low | |
| Q9Y2H0 | G24ef8 | KIAA0964 protein | 279.6086 | 310 | 226.545 | 271.9604 | −3.04E−01 | 1.48E−01 | 1 | Unchanged Low | |
| Q9UJT9 | E15cd8 | F-box and leucine-rich repeat pr | 287.9128 | 294 | 226.762 | 269.5315 | −3.44E−01 | 2.98E−02 | 1 | Unchanged Low | |
| Q9Y306 | O23ef2 | pelota homolog (Drosophila) | 146.8037 | 190 | 226.8083 | 187.7391 | 6.28E−01 | 3.69E−01 | 1 | Unchanged Low | |
| Q9Y6A6 | N04ef3 | transcription factor (p38 interacti | 174.5425 | 171 | 226.9539 | 190.8287 | 3.79E−01 | −2.97E−02 | 1 | Unchanged Low | |
| O75300 | K18cd2 | ribosome binding protein 1 homo | 98.67954 | 73.7 | 227.0651 | 133.1515 | 1.20E+00 | −4.21E−01 | 1 | Unchanged Low | |
| P51798 | L03ef1 | chloride channel 7 | 278.4282 | 282 | 228.319 | 263.0106 | −2.86E−01 | 1.98E−02 | 1 | Unchanged Low | |
| Q14839 | C21ab4 | chromodomain helicase DNA bin | 104.8847 | 161 | 228.322 | 164.8091 | 1.12E+00 | 6.20E−01 | 1 | Unchanged Low | |
| O60299 | O18gh1 | KIAA0552 gene product | 132.5981 | 337 | 228.426 | 232.6614 | 7.85E−01 | 1.35E+00 | 1 | Unchanged Low | |
| Q9Y6F1 | H03ab2 | ADP-ribosyltransferase (NAD+: p | 248.9844 | 399 | 228.5355 | 292.1577 | −1.24E−01 | 6.80E−01 | 1 | Unchanged Low | |
| O15212 | E04ef3 | HLA class II region expressed ge | 250.113 | 292 | 228.5629 | 256.7661 | −1.30E−01 | 2.22E−01 | 1 | Unchanged Low | |
| Q9Y2W2 | J14ef2 | WW domain binding protein 11 | 335.0512 | 304 | 228.6314 | 289.1388 | −5.51E−01 | −1.42E−01 | 1 | Unchanged Low | |
| Q9UKN5 | O03cd8 | PR domain containing 4 | 252.6208 | 60.6 | 228.639 | 187.2825 | −1.44E−01 | −1.65E+00 | 1 | Unchanged Low | |
| Q00978 | C24cd6 | interferon-stimulated transcription | 303.7171 | 404 | 228.7768 | 312.2774 | −4.09E−01 | 4.13E−01 | 1 | Unchanged Low | |
| Q9UHY0 | L08ef1 | HP1-BP74 | 295.9512 | 267 | 228.8435 | 263.8122 | −3.71E−01 | −1.50E−01 | 1 | Unchanged Low | |
| Q9Y248 | L13ef2 | HSPC037 protein | 92.39515 | 70.3 | 229.0541 | 130.5748 | 1.31E+00 | −3.95E−01 | 1 | Unchanged Low | |
| O96019 | E21ab2 | BAF53 | 228.9673 | 345 | 229.5696 | 267.9206 | 3.79E−03 | 5.92E−01 | 1 | Unchanged Low | |
| P10276 | K19ab8 | retinoic acid receptor: alpha | 250.0312 | 122 | 229.7466 | 200.7048 | −1.22E−01 | −1.03E+00 | 1 | Unchanged Low | |
| Q9BUC7 | P22gh8 | hypothetical protein MGC4172 | 136.4413 | 398 | 229.8471 | 254.8265 | 7.52E−01 | 1.55E+00 | 1 | Unchanged Low | |
| Q9HAC1 | D01gh5 | praja 1 | 143.4188 | 97.5 | 230.0154 | 156.9785 | 6.81E−01 | −5.57E−01 | 1 | Unchanged Low | |
| Q93078 | M18gh6 | H2B histone family: member H | 179.9656 | 203 | 230.2553 | 204.3058 | 3.56E−01 | 1.72E−01 | 1 | Unchanged Low | |
| P08572 | N22ef6 | collagen; type IV: alpha 2 | 70.14126 | 87.3 | 230.3868 | 129.2665 | 1.72E+00 | 3.15E−01 | 1 | Unchanged Low | |
| P15056 | G10ef5 | v-raf marine sarcoma viral oncog | 194.1803 | 174 | 231.0687 | 199.7155 | 2.51E−01 | −1.59E−01 | 1 | Unchanged Low | |
| Q15543 | J15cd2 | TAF13 RNA polymerase II: TATA | 222.3129 | 262 | 231.2695 | 238.5733 | 5.70E−02 | 2.38E−01 | 1 | Unchanged Low | |
| O14901 | J05cd3 | TGFB inducible early growth res | 277.9069 | 277 | 231.5621 | 262.1749 | −2.63E−01 | −4.43E−03 | 1 | Unchanged Low | |
| | N10gh8 | diphosphate dimethylallyl diphos | 313.8915 | 257 | 231.841 | 267.6704 | −4.37E−01 | −2.87E−01 | 1 | Unchanged Low | |
| P51965 | O13cd3 | ubiquitin-conjugating enzyme E2 | 254.2693 | 673 | 232.1178 | 386.4499 | −1.32E−01 | 1.40E+00 | 1 | Unchanged Low | |
| Q9Y3R2 | G04cd8 | transportin-SR | 338.45 | 219 | 232.2301 | 263.1006 | −5.43E−01 | −6.31E−01 | 1 | Unchanged Low | |
| O94829 | G10gh1 | importin 13 | 160.8646 | 247 | 232.3109 | 213.3188 | 5.30E−01 | 6.17E−01 | 1 | Unchanged Low | |
| Q92859 | M13ab8 | neogenin homolog 1 (chicken) | 153.0455 | 249 | 232.6926 | 211.5326 | 6.04E−01 | 7.01E−01 | 1 | Unchanged Low | |
| Q12846 | O02cd2 | syntaxin 4A (placental) | 323.5547 | 93.2 | 232.9973 | 216.5887 | −4.74E−01 | −1.80E+00 | 1 | Unchanged Low | |
| P01286 | G15ef1 | growth hormone releasing hormo | 237.6384 | 263 | 233.028 | 244.6261 | −2.83E−02 | 1.47E−01 | 1 | Unchanged Low | |
| Q9UH92 | L01cd8 | transcription factor-like 4 | 172.7135 | 195 | 233.1639 | 200.1328 | 4.33E−01 | 1.72E−01 | 1 | Unchanged Low | |
| O75323 | P09ab5 | glioblastoma amplified sequence | 178.3893 | 168 | 233.5843 | 193.2783 | 3.89E−01 | −8.78E−02 | 1 | Unchanged Low | |
| Q9UQ33 | P07ef2 | microtubule-associated protein: | 101.7866 | 134 | 234.2195 | 156.5563 | 1.20E+00 | 3.93E−01 | 1 | Unchanged Low | |
| Q9Y3A7 | A17ef2 | CGI-101 protein | 402.1639 | 210 | 234.3057 | 282.2981 | −7.79E−01 | −9.34E−01 | 1 | Unchanged Low | |
| Q9Y2Z0 | A17cd7 | suppressor of G2 allele of SKP1; | 184.6693 | 258 | 234.3223 | 224.9662 | 3.44E−01 | 4.71E−01 | 1 | Unchanged Low | |
| P56211 | F16ab2 | cyclic AMP phosphoprotein: 19 k | 276.9734 | 386 | 234.3401 | 299.2676 | −2.41E−01 | 4.81E−01 | 1 | Unchanged Low | |
| P21127 | O13ef1 | cell division cycle 2-like 2 | 159.1108 | 304 | 234.454 | 232.4012 | 5.59E−01 | 9.32E−01 | 1 | Unchanged Low | |
| Q07866 | B23ab7 | kinesin 2 (60-70kD) | 254.5932 | 408 | 234.5156 | 299.1796 | −1.19E−01 | 6.82E−01 | 1 | Unchanged Low | |
| Q9BTI3 | H11ef8 | KIAA1068 protein | 228.7025 | 230 | 234.5211 | 231.0835 | 3.62E−02 | 8.33E−03 | 1 | Unchanged Low | |
| Q9H2J4 | E11gh6 | hypothetical protein MGC3062 | 238.7573 | 256 | 235.0353 | 243.2412 | −2.27E−02 | 1.00E−01 | 1 | Unchanged Low | |
| Q9NXE5 | E10gh2 | hypothetical protein FLJ20296 | 193.8379 | 399 | 235.1468 | 275.9024 | 2.79E−01 | 1.04E+00 | 1 | Unchanged Low | |
| Q92572 | E17ab4 | adaptor-related protein complex: | 86.23952 | 209 | 235.2813 | 176.703 | 1.45E+00 | 1.27E+00 | 1 | Unchanged Low | |
| Q9NXT6 | K05gh2 | FtsJ homolog 3 (E. coli) | 254.7485 | 277 | 235.4026 | 255.685 | −1.14E−01 | 1.20E−01 | 1 | Unchanged Low | |
| O15321 | F05cd6 | transmembrane 9 superfamily m | 295.1129 | 227 | 235.695 | 252.5647 | −3.24E−01 | −3.79E−01 | 1 | Unchanged Low | |
| O15383 | F07cd6 | HIV-1 Tat interactive protein 2: 3 | 402.8554 | 444 | 235.7849 | 360.7235 | −7.73E−01 | 1.39E−01 | 1 | Unchanged Low | |
| O60403 | B06cd8 | olfactory receptor: family 10: sub | 237.7465 | 262 | 235.906 | 245.2774 | −1.12E−02 | 1.41E−01 | 1 | Unchanged Low | |
| P49354 | K24ab4 | farnesyltransferase: CAAX box; | 173.9344 | 193 | 235.9155 | 201.1077 | 4.40E−01 | 1.54E−01 | 1 | Unchanged Low | |
| P48039 | B07ef6 | melatonin receptor 1A | 305.0267 | 208 | 236.1249 | 249.7986 | −3.69E−01 | −5.51E−01 | 1 | Unchanged Low | |
| O43562 | C08ab8 | solute carrier family 22 (organic | 115.4101 | 125 | 236.1587 | 158.9844 | 1.03E+00 | 1.20E−01 | 1 | Unchanged Low | |
| Q9HC89 | N24ef8 | calpain 10 | 212.0136 | 242 | 236.247 | 230.2337 | 1.56E−01 | 1.93E−01 | 1 | Unchanged Low | |
| P00790 | M13ef1 | pepsinogen 5: group I (pepsinog | 256.009 | 266 | 236.6918 | 253.0491 | −1.13E−01 | 5.77E−02 | 1 | Unchanged Low | |
| Q9NVP1 | G01cd6 | DEAD/H (Asp-Glu-Ala-Asp/His) | 78.40555 | 89 | 236.7778 | 134.724 | 1.59E+00 | 1.63E−01 | 1 | Unchanged Low | |
| Q9UBN1 | F23ef3 | calcium channel: voltage-depend | 269.9719 | 360 | 237.0557 | 289.0967 | −1.88E−01 | 4.16E−01 | 1 | Unchanged Low | |
| P35659 | M22cd3 | DEK oncogene (DNA binding) | 60.02159 | 77.8 | 237.1934 | 131.6712 | 1.57E+00 | −4.06E−02 | 1 | Unchanged Low | |
| P54252 | N09ef6 | Machado-Joseph disease (spino | 156.7549 | 274 | 237.2775 | 222.5126 | 5.98E−01 | 8.03E−01 | 1 | Unchanged Low | |
| Q14152 | L12cd3 | eukaryotic translation initiation fa | 364.5131 | 268 | 237.302 | 269.959 | −6.19E−01 | −4.43E−01 | 1 | Unchanged Low | |
| Q9Y314 | El6ef2 | eNOS interacting protein | 123.3313 | 219 | 237.3058 | 193.3594 | 9.44E−01 | 8.31E−01 | 1 | Unchanged Low | |
| Q9NRC7 | E23ef3 | sirtuin silent mating type informal | 136.0486 | 328 | 237.5194 | 233.7238 | 8.04E−01 | 1.27E+00 | 1 | Unchanged Low | |
| P02533 | N02ef6 | keratin 14 (epidermolysis bullosa | 203.4102 | 190 | 237.6951 | 210.4458 | 2.25E−01 | −9.66E−02 | 1 | Unchanged Low | |
| O60907 | J19cd2 | transducin (beta)-like 1 X-linked | 175.9553 | 171 | 237.7396 | 194.8701 | 4.34E−01 | −4.19E−02 | 1 | Unchanged Low | |
| P43155 | B16ab3 | camitine acetyltransferase | 288.355 | 190 | 237.8908 | 238.6829 | −2.78E−01 | −6.03E−01 | 1 | Unchanged Low | |
| Q15077 | H06ab8 | pyrimidinergic receptor P2Y: G-p | 389.9746 | 248 | 238.1475 | 292.1056 | −7.12E−01 | −6.52E−01 | 1 | Unchanged Low | |
| Q12840 | I06ab7 | kinesin family member 5A | 238.3605 | 282 | 238.2407 | 252.8959 | −7.25E−04 | 2.43E−01 | 1 | Unchanged Low | |
| P26599 | A05cd3 | polypyrimidine tract binding prote | 160.4912 | 345 | 236.4 | 247.8557 | 5.71E−01 | 1.10E+00 | 1 | Unchanged Low | |
| Q9Y316 | M11ef2 | C21orf19-like protein | 450.6396 | 327 | 238.5981 | 338.8101 | −9.17E−01 | −4.62E−01 | 1 | Unchanged Low | |
| P24394 | O18ab6 | interleukin 4 receptor | 309.5199 | 330 | 238.6201 | 292.7071 | −3.75E−01 | 9.24E−02 | 1 | Unchanged Low | |
| P32418 | J22gh1 | solute carrier family 8 (sodium/ca | 171.4835 | 134 | 236.7225 | 181.3894 | 4.77E−01 | −3.56E−01 | 1 | Unchanged Low | |
| P42694 | N01ef3 | helicase with zinc finger domain | 165.7017 | 146 | 238.7561 | 183.6122 | 5.27E−01 | −1.79E−01 | 1 | Unchanged Low | |
| O60755 | L09cd3 | galanin receptor 3 | 162.6185 | 268 | 238.7669 | 223.2368 | 5.54E−01 | 7.22E−01 | 1 | Unchanged Low | |
| Q16204 | A18cd4 | DNA segment: single copy: prob | 192.0586 | 225 | 238.8166 | 218.6477 | 3.14E−01 | 2.29E−01 | 1 | Unchanged Low | |
| Q12980 | N15cd7 | Conserved gene telomeric to alpl | 183.7714 | 174 | 236.9231 | 198.8702 | 3.79E−01 | −7.95E−02 | 1 | Unchanged Low | |
| P08559 | N16ab7 | pyruvate dehydrogenase (lipoam | 375.1479 | 206 | 239.4879 | 273.612 | −6.48E−01 | −8.63E−01 | 1 | Unchanged Low | |
| Q9Y2U9 | K02ef3 | host cell factor homolog | 127.5142 | 154 | 239.623 | 173.6438 | 9.10E−01 | 2.70E−01 | 1 | Unchanged Low | |
| Q9Y2T2 | P17cd7 | adaptor-related protein complex | 336.9686 | 395 | 239.6299 | 323.8714 | −4.92E−01 | 2.29E−01 | 1 | Unchanged Low | |
| Q13105 | K10cd3 | zinc finger protein 151 (pHZ-67) | 177.9186 | 243 | 240.018 | 220.1539 | 4.32E−01 | 4.47E−01 | 1 | Unchanged Low | |
| Q9H1J1 | J12gh5 | similar to yeast Upf3: variant A | 154.12 | 183 | 241.0316 | 192.8261 | 6.45E−01 | 2.50E−01 | 1 | Unchanged Low | |
| Q08752 | E01cd1 | peptidylprolyl isomerase D (cycl | 446.371 | 203 | 241.0389 | 296.8872 | −8.89E−01 | −1.13E+00 | 1 | Unchanged Low | |
| P38159 | F14ab7 | RNA binding motif protein: X chr | 342.4693 | 176 | 241.0429 | 253.2584 | −5.07E−01 | −9.58E−01 | 1 | Unchanged Low | |
| Q9Y337 | A12cd8 | kallikrein 5 | 215.5515 | 312 | 241.2144 | 256.4089 | 1.62E−01 | 5.36E−01 | 1 | Unchanged Low | |
| P25445 | A02ef6 | tumor necrosis factor receptor su | 102.3524 | 215 | 241.2257 | 186.0304 | 1.24E+00 | 1.07E+00 | 1 | Unchanged Low | |
| Q9BZH1 | P02gh5 | membrane-spanning 4-domains: | 350.8825 | 212 | 241.5154 | 268.2725 | −5.39E−01 | −7.24E−01 | 1 | Unchanged Low | |
| O43633 | O12ef4 | putative breast adenocarcinoma | 308.4601 | 232 | 241.573 | 260.7188 | −3.53E−01 | −4.10E−01 | 1 | Unchanged Low | |
| Q99536 | B03cd6 | vesicle amine transport protein 1 | 22.8504 | 400 | 241.6698 | 288.1564 | 1.17E−01 | 8.44E−01 | 1 | Unchanged Low | |
| P24557 | N11cd1 | thromboxane A synthase 1 (plate | 326.4482 | 236 | 241.6841 | 268.1689 | −4.34E−01 | −4.66E−01 | 1 | Unchanged Low | |
| O75369 | G16ab4 | filamin B; beta (actin binding pro | 331.6975 | 459 | 241.7672 | 344.1714 | −4.56E−01 | 4.69E−01 | 1 | Unchanged Low | |
| P50453 | H10ab8 | serine (or cysteine) proteinase in | 439.8605 | 370 | 242.4005 | 350.7941 | −8.60E−01 | −2.49E−01 | 1 | Unchanged Low | |
| P35680 | O18cd1 | transcription factor 2; hepatic: LF | 235.5021 | 174 | 242.5943 | 217.2497 | 4.28E−02 | −4.40E−01 | 1 | Unchanged Low | |
| Q03924 | I01gh7 | zinc finger protein 117 (HPF9) | 257.6571 | 301 | 242.7729 | 267.2646 | −8.70E−02 | 2.24E−01 | 1 | Unchanged Low | |
| Q9NRQ5 | F17gh4 | FNS protein | 361.924 | 325 | 242.8257 | 309.8867 | −5.76E−01 | −1.56E−01 | 1 | Unchanged Low | |
| Q99867 | G12gh7 | tubulin; beta polypeptide 4: mem | 376.7361 | 247 | 243.6773 | 289.1694 | −6.29E−01 | −6.08E−01 | 1 | Unchanged Low | |
| Q9H682 | B19gh7 | A20-binding inhibitor of NF-kapp | 205.6095 | 365 | 243.7949 | 271.5091 | 2.46E−01 | 8.28E−01 | 1 | Unchanged Low | |
| O00148 | N12cd5 | DEAD/H (Asp-Glu-Ala-Asp/His) | 259.196 | 140 | 244.0023 | 214.3036 | −8.71E−02 | −8.92E−01 | 1 | Unchanged Low | |
| | F11cd8 | TCF3 (E2A) fusion partner (in ch | 170.7519 | 295 | 244.0153 | 236.7141 | 5.15E−01 | 7.91E−01 | 1 | Unchanged Low | |
| Q9UG51 | N05ef3 | anaphase-promoting complex su | 274.4079 | 331 | 244.0324 | 283.0875 | −1.69E−01 | 2.70E−01 | 1 | Unchanged Low | |
| O75175 | F12cd8 | CCR4-NOT transcription comple | 230.1579 | 253 | 244.0384 | 242.329 | 8.45E−02 | 1.35E−01 | 1 | Unchanged Low | |
| Q9GZQ8 | C05gh2 | microtubute-associated proteins | 261.5987 | 395 | 244.0725 | 300.1702 | −1.00E−01 | 5.94E−01 | 1 | Unchanged Low | |
| P19438 | A04ef6 | tumor necrosis factor receptor su | 233.3188 | 197 | 244.3384 | 224.9565 | 6.66E−02 | −2.43E−01 | 1 | Unchanged Low | |
| Q9NRR8 | O04gh4 | small protein effector 1 of Cdc42 | 338.2133 | 329 | 244.5729 | 303.9389 | −4.68E−01 | −3.97E−02 | 1 | Unchanged Low | |
| O95162 | N10ef8 | peroxisomal short-chain alcohol | 4154.9486 | 108 | 244.9155 | 169.1352 | 6.60E−01 | −5.27E−01 | 1 | Unchanged Low | |
| O95279 | J22cd3 | potassium channel: subfamily K: | 274.3417 | 127 | 244.9972 | 215.5542 | −1.63E−01 | −1.11E+00 | 1 | Unchanged Low | |
| Q9NTW4 | N21ef8 | DKFZP564A2416 protein | 403.4188 | 219 | 245.1953 | 289.1446 | −7.18E−01 | −8.83E−01 | 1 | Unchanged Low | |
| Q9NWM9 | H21gh2 | hypothetical protein FLJ20730 | 121.538 | 207 | 245.69 | 191.4607 | 1.02E+00 | 7.69E−01 | 1 | Unchanged Low | |
| P40121 | P23ab5 | capping protein (actin filament): | 452.2006 | 414 | 245.8846 | 370.7982 | −8.79E−01 | −1.26E−01 | 1 | Unchanged Low | |
| Q92466 | I04ab3 | damage-specific DNA binding pr | 99.17314 | 220 | 246.0478 | 188.2839 | 1.31E+00 | 1.15E+00 | 1 | Unchanged Low | |
| O43365 | K05gh7 | homeo box A3 | 132.7369 | 196 | 246.2071 | 191.5647 | 8.91E−01 | 5.60E−01 | 1 | Unchanged Low | |
| Q9NVJ7 | O02gh3 | DEAD/H (Asp-Glu-Ala-Asp/His) | 336.1927 | 284 | 246.3831 | 288.7892 | −4.48E−01 | −2.44E−01 | 1 | Unchanged Low | |
| Q9NVY5 | H08ef7 | serologically defined colon cance | 182.4596 | 178 | 246.7633 | 202.4155 | 4.36E−01 | −3.55E−02 | 1 | Unchanged Low | |
| Q9BY40 | G12gh8 | CDA11 protein | 141.0763 | 131 | 246.8975 | 173.0132 | 8.07E−01 | −1.06E−01 | 1 | Unchanged Low | |
| Q9NZE2 | O06ef2 | hypothetical protein LOC51321 | 76.41026 | 135 | 247.0025 | 152.875 | 1.69E+00 | 8.23E−01 | 1 | Unchanged Low | |
| P29275 | K11ab2 | adenosine A2b receptor | 354.5115 | 191 | 247.0615 | 264.0659 | −5.21E−01 | −8.95E−01 | 1 | Unchanged Low | |
| Q9NZE0 | P22ef1 | WW domain-containing adapter | 82.72809 | 71.1 | 247.6094 | 133.8159 | 1.58E+00 | −2.18E−01 | 1 | Unchanged Low | |
| Q03111 | N03ab7 | myeloid/lymphoid or mixed-lineag | 225.1377 | 162 | 247.8275 | 211.7891 | 1.39E−01 | −4.71E−01 | 1 | Unchanged Low | |
| Q08499 | G21ef6 | phosphodiesterase 4D: cAMP-sp | 125.5177 | 92.9 | 248.1727 | 155.5405 | 9.83E−01 | −4.34E−01 | 1 | Unchanged Low | |
| Q15628 | A20ef6 | TNFRSF1A-associated via death | 214.1641 | 250 | 248.2613 | 237.3623 | 2.13E−01 | 2.21E−01 | 1 | Unchanged Low | |
| Q9Y657 | C07cd7 | spindlin | 217.9487 | 202 | 248.4185 | 222.9201 | 1.89E−01 | −1.07E−01 | 1 | Unchanged Low | |
| Q9UHQ1 | I09cd8 | nuclear prelamin A recognition fa | 312.6614 | 303 | 248.5926 | 288.0624 | −3.32E−01 | −4.84E−02 | 1 | Unchanged Low | |
| P17980 | H24ef6 | proteasome (prosome: macropai | 331.1488 | 193 | 248.6286 | 257.496 | −4.13E−01 | −7.81E−01 | 1 | Unchanged Low | |
| P45877 | I19ab8 | peptidylprolyl isomerase C (cycl | 165.0716 | 169 | 248.9851 | 194.3167 | 5.93E−01 | 3.30E−02 | 1 | Unchanged Low | |
| O95340 | A23cd5 | 3′-phosphoadenosine 5′-phospho | 110.9876 | 88.6 | 249.0622 | 149.5355 | 1.17E+00 | −3.26E−01 | 1 | Unchanged Low | |
| Q9H9Z7 | F18gh5 | hypothetical protein FLJ12448 | 158.5794 | 279 | 249.1478 | 228.8009 | 6.52E−01 | 8.13E−01 | 1 | Unchanged Low | |
| O15172 | E11cd4 | phosphoserine phosphatase-like | 321.9068 | 291 | 249.3629 | 287.4495 | −3.68E−01 | −1.45E−01 | 1 | Unchanged Low | |
| P01298 | F05ab8 | pancreatic polypeptide | 544.0034 | 194 | 249.376 | 329.1527 | −1.13E+00 | −1.49E+00 | 1 | Unchanged Low | |
| Q13395 | J17cd2 | TAR (HIV) RNA binding protein 1 | 260.7056 | 234 | 249.3777 | 248.0711 | −6.41E−02 | −1.55E−01 | 1 | Unchanged Low | |
| O15551 | G23ab4 | claudin 3 | 279.9519 | 250 | 249.4618 | 259.744 | −1.66E−01 | −1.64E−01 | 1 | Unchanged Low | |
| P55060 | G20ef6 | CSE1 chromosome segregation | 322.1588 | 276 | 249.5903 | 282.6907 | −3.68E−01 | −2.21E−01 | 1 | Unchanged Low | |
| O43286 | E13ab3 | UDP-Gal:betaGlcNAc beta 1; 4- | 316.581 | 468 | 249.8072 | 344.9578 | −3.42E−01 | 5.65E−01 | 1 | Unchanged Low | |
| P04424 | J10ab2 | argininosuccinate lyase | 594.9833 | 269 | 250.046 | 371.2796 | −1.25E+00 | −1.15E+00 | 1 | Unchanged Low | |
| Q9NWR5 | C10gh3 | hypothetical protein FLJ20857 | 171.9643 | 252 | 250.1961 | 224.6496 | 5.41E−01 | 5.50E−01 | 1 | Unchanged Low | |
| Q9BUN8 | M21gh6 | hypothetical protein MGC3067 | 165.6904 | 175 | 250.2847 | 197.0059 | 5.95E−01 | 7.92E−02 | 1 | Unchanged Low | |
| P49760 | G01ab4 | CDC-like kinase 2 | 69.23701 | 97.1 | 250.3266 | 138.8925 | 1.85E+00 | 4.88E−01 | 1 | Unchanged Low | |
| Q9UEG3 | E01gh7 | myotubularin related protein 3 | 367.623 | 236 | 250.553 | 284.5589 | −5.53E−01 | −6.42E−01 | 1 | Unchanged Low | |
| Q9P0Y6 | N07ef1 | chromosome 3 open rending fra | 268.1158 | 256 | 250.6098 | 258.2636 | −9.74E−02 | −6.63E−02 | 1 | Unchanged Low | |
| Q92535 | M06ab8 | phosphatidytinositol glycan: class | 218.2502 | 172 | 250.9192 | 213.6071 | 2.01E−01 | −3.46E−01 | 1 | Unchanged Low | |
| Q00577 | A24gh7 | purine-rich element binding prote | 839.6 | 273 | 251.0214 | 454.6488 | −1.74E+00 | −1.62E+00 | 1 | Unchanged Low | |
| O75717 | L21ab2 | AND-1 protein | 225.9974 | 250 | 251.1522 | 242.2724 | 1.52E−01 | 1.44E−0l | 1 | Unchanged Low | |
| P04278 | L09cd1 | sex hormone-binding globulin | 215.6067 | 196 | 251.1944 | 221.0173 | 2.20E−01 | −1.36E−01 | 1 | Unchanged Low | |
| P51124 | G19ab5 | granzyme M (lymphocyte met-as | 108.9072 | 114 | 251.2498 | 158.1827 | 1.21E+00 | 7.09E−02 | 1 | Unchanged Low | |
| P15259 | P04ab7 | phosphoglycerate mutase 2 (mus | 333.2442 | 363 | 251.6606 | 315.8301 | −4.05E−01 | 1.22E−01 | 1 | Unchanged Low | |
| Q12893 | O13cd7 | PL6 protein | 174.3077 | 280 | 252.0241 | 235.3565 | 5.32E−01 | 6.82E−01 | 1 | Unchanged Low | |
| P57738 | A11gh2 | T-ceII leukemia translocation alt | 186.5683 | 232 | 252.2165 | 217.0429 | 5.99E−01 | 4.80E−01 | 1 | Unchanged Low | |
| P24863 | O22ef5 | cyclin C | 668.8634 | 523 | 252.3632 | 481.4089 | −1.41E+00 | −3.55E−01 | 1 | Unchanged Low | |
| Q9Y370 | H03ef2 | CGI-60 protein | 153.6349 | 157 | 252.5036 | 187.603 | 7.17E−01 | 2.82E−02 | 1 | Unchanged Low | |
| Q9HD89 | E04gh4 | found in inflammatory zone 3 | 189.8748 | 182 | 252.7224 | 208.2163 | 4.13E−01 | −6.07E−02 | 1 | Unchanged Low | |
| O14807 | N04cd7 | muscle RAS oncogene homolog | 105.1243 | 181 | 252.8147 | 179.5238 | 1.27E+00 | 7.81E−01 | 1 | Unchanged Low | |
| Q13435 | I07cd7 | splicing factor 3b; subunit 2; 145 | 270.3354 | 268 | 252.8473 | 263.6077 | −9.65E−02 | −1.45E−02 | 1 | Unchanged Low | |
| Q9UMY4 | F19cd8 | sorting nexin 12 | 178.8659 | 227 | 253.0136 | 219.6698 | 5.00E−01 | 3.45E−01 | 1 | Unchanged Low | |
| O43929 | C04ab8 | origin recognition complex: subu | 267.5605 | 354 | 253.265 | 291.5955 | −7.92E−02 | 4.04E−01 | 1 | Unchanged Low | |
| Q9BZQ1 | H14gh7 | chromosome 1 open reading fra | 219.6632 | 362 | 253.5183 | 278.4192 | 2.07E−01 | 7.21E−01 | 1 | Unchanged Low | |
| P55042 | K03ef5 | Ras-retated associated with diab | 300.5794 | 514 | 253.5279 | 355.8906 | −2.46E−01 | 7.73E−01 | 1 | Unchanged Low | |
| Q92870 | K18ef1 | amyloid beta (A4) precursor prot | 76.07104 | 77.9 | 253.5714 | 135.8422 | 1.74E+00 | 3.40E−02 | 1 | Unchanged Low | |
| P33778 | C19gh7 | H2B histone family: member F | 169.4431 | 391 | 253.9554 | 271.5522 | 5.84E−01 | 1.21E+00 | 1 | Unchanged Low | |
| Q9ULZ3 | K22cd8 | apoptosis-associaled speck-like | 255.9554 | 517 | 254.0856 | 342.2046 | −1.06E−02 | 1.01E+00 | 1 | Unchanged Low | |
| Q13485 | A20ef5 | MAD: mothers against decapenta | 163.1373 | 181 | 254.1546 | 199.4935 | 6.40E−01 | 1.51E−01 | 1 | Unchanged Low | |
| Q9H684 | C24gh5 | zinc finger protein 335 | 326.6826 | 261 | 254.3385 | 280.5396 | −3.61E−01 | −3.26E−01 | 1 | Unchanged Low | |
| Q9H7F8 | O06gh7 | hypothetical protein FLJ20958 | 190.1073 | 258 | 254.7196 | 234.2579 | 4.22E−01 | 4.40E−01 | 1 | Unchanged Low | |
| Q92508 | B17gh1 | KIAA0233 gene product | 241.1859 | 216 | 255.0491 | 237.2652 | 8.06E−02 | −1.62E−01 | 1 | Unchanged Low | |
| Q9UM47 | C15ab8 | Notch homolog 3 (Drosophila) | 64.97745 | 132 | 255.0839 | 150.552 | 1.97E+00 | 1.02E+00 | 1 | Unchanged Low | |
| P18084 | E17ef7 | integrin: beta 5 | 259.2818 | 129 | 255.1335 | 214.6124 | −2.33E−02 | −1.00E+00 | 1 | Unchanged Low | |
| Q99462 | K22gh6 | ubiquitin-conjugating enzyme E2 | 207.6691 | 179 | 255.2632 | 213.9424 | 2.98E−01 | −2.15E−01 | 1 | Unchanged Low | |
| O75619 | J10ef7 | RNA heticase-related protein | 198.1059 | 335 | 255.6713 | 262.7733 | 3.68E−01 | 7.56E−01 | 1 | Unchanged Low | |
| Q92733 | K07cd1 | papillary renal cell carcinoma (tra | 200.3207 | 260 | 256.1407 | 238.8334 | 3.55E−01 | 3.76E−01 | 1 | Unchanged Low | |
| Q15796 | H21ab7 | MAD: mothers against decapenta | 376.8422 | 446 | 256.2171 | 359.5423 | −5.57E−01 | 2.42E−01 | 1 | Unchanged Low | |
| P15291 | E07ab3 | UDP-Gal:betaGlcNAc beta 1;4- | 411.9325 | 403 | 256.9006 | 357.1444 | −6.81E−01 | −3.31E−02 | 1 | Unchanged Low | |
| Q99417 | I07cd8 | c-myc binding protein | 264.1386 | 256 | 256.9088 | 259.037 | −4.00E−02 | −4.48E−02 | 1 | Unchanged Low | |
| P06732 | D09ab4 | creatine kinase; muscle | 202.527 | 127 | 257.2272 | 195.4365 | 3.45E−01 | −6.78E−01 | 1 | Unchanged Low | |
| P50570 | K13ab5 | dynamin 2 | 248.1389 | 228 | 257.2474 | 244.4335 | 5.20E−02 | −1.23E−01 | 1 | Unchanged Low | |
| Q9Y5A9 | F20ef2 | high-glucose-regulated protein 8 | 359.385 | 371 | 257.3917 | 329.2557 | −4.82E−01 | 4.59E−02 | 1 | Unchanged Low | |
| Q92841 | B17cd6 | DEAD/H (Asp-Glu-Ala-Asp/His) t | 229.8916 | 270 | 257.9255 | 252.4572 | 1.66E−01 | 2.30E−01 | 1 | Unchanged Low | |
| P51808 | B22cd2 | t-complex-associated-testis-expr | 182.2992 | 245 | 257.9487 | 228.2619 | 5.01E−01 | 4.24E−01 | 1 | Unchanged Low | |
| O95758 | H23cd5 | ROD1 regulator of differentiation | 411.9888 | 462 | 258.1758 | 377.5392 | −6.74E−01 | 1.67E−01 | 1 | Unchanged Low | |
| P32970 | I15ef5 | tumor necrosis factor (ligand) sup | 443.4158 | 456 | 258.4229 | 386.0177 | −7.79E−01 | 4.11E−02 | 1 | Unchanged Low | |
| O75689 | K21cd7 | centaurin: alpha 1 | 191.7263 | 327 | 258.6362 | 259.1653 | 4.32E−01 | 7.71E−01 | 1 | Unchanged Low | |
| P55072 | O06cd4 | valosin-containing protein | 466.7034 | 363 | 258.8411 | 362.8018 | −8.50E−01 | −3.63E−01 | 1 | Unchanged Low | |
| P47813 | A16ab4 | eukaryotic translation initiation fa | 486.6493 | 337 | 259.0481 | 360.7943 | −9.10E−01 | −5.31E−01 | 1 | Unchanged Low | |
| Q15532 | J03cd2 | synovial sarcoma translocation: | 190.8535 | 212 | 259.1488 | 220.5805 | 4.41E−01 | 1.50E−01 | 1 | Unchanged Low | |
| Q15813 | M19cd2 | tubulin-specific chaperone e | 151.2337 | 166 | 259.2747 | 192.1296 | 7.78E−01 | 1.33E−01 | 1 | Unchanged Low | |
| P14373 | B08cd2 | ret finger protein | 282.5757 | 270 | 259.3599 | 270.5765 | −1.24E−01 | −6.68E−02 | 1 | Unchanged Low | |
| O14980 | G02cd3 | exportin 1 (CRM1 homolog: yeas | 239.4965 | 207 | 259.7187 | 235.5264 | 1.17E−01 | −2.08E−01 | 1 | Unchanged Low | |
| P07942 | P20ef6 | laminin: beta 1 | 71.03574 | 92.4 | 259.7579 | 141.0764 | 1.87E+00 | 3.80E−01 | 1 | Unchanged Low | |
| P53041 | C20cd1 | protein phosphatase 5: catalytic | 272.5094 | 196 | 259.8136 | 242.7533 | −6.88E−02 | 4.76E−01 | 1 | Unchanged Low | |
| Q07820 | E12ef6 | myeloid cell leukemia sequence | 362.5007 | 243 | 260.0329 | 288.4688 | 4.79E−01 | −5.78E−01 | 1 | Unchanged Low | |
| P53816 | E04cd7 | HRAS-like suppressor 3 | 606.0957 | 558 | 260.1565 | 474.8201 | −1.22E+00 | −1.19E−01 | 1 | Unchanged Low | |
| Q9P1C8 | E09gh4 | hypothetical protein PRO2577 | 238.9909 | 205 | 280.3147 | 234.8631 | 1.23E−01 | −2.19E−01 | 1 | Unchanged Low | |
| P51966 | O21cd3 | ubiquitin-conjugating enzyme E2 | 148.3528 | 243 | 260.3185 | 217.1092 | 8.11E−01 | 7.10E−01 | 1 | Unchanged Low | |
| Q99739 | M03cd7 | pim-2 oncogene | 178.694 | 274 | 260.4482 | 237.5556 | 5.44E−01 | 6.14E−01 | 1 | Unchanged Low | |
| P35913 | N14ab7 | phosphodiesterase 6B: cGMP-sp | 199.3654 | 173 | 260.5708 | 210.8879 | 3.86E−01 | −2.07E−01 | 1 | Unchanged Low | |
| P52756 | J24cd5 | RNA binding motif protein 5 | 280.6537 | 230 | 261.0637 | 257.2441 | −1.04E−01 | −2.87E−01 | 1 | Unchanged Low | |
| Q9NXX5 | K17gh3 | hypothetical protein FLJ20004 | 130.163 | 197 | 261.2642 | 196.0576 | 1.01E+00 | 5.96E−01 | 1 | Unchanged Low | |
| Q14498 | O06cd5 | RNA-binding region (RNP1: RRM | 196.0463 | 216 | 261.5829 | 224.4664 | 4.16E−01 | 1.38E−01 | 1 | Unchanged Low | |
| P28069 | A03ab8 | POU domain: class 1: transcriptio | 261.1822 | 206 | 261.7763 | 243.064 | 3.28E−03 | −3.41E−01 | 1 | Unchanged Low | |
| P51397 | E20ab5 | death-associated protein | 348.1009 | 312 | 261.9342 | 307.4499 | −4.10E−01 | −1.57E−01 | 1 | Unchanged Low | |
| Q9NUI8 | C24gh7 | putative methyltransferase | 157.3551 | 329 | 262.0287 | 249.4637 | 7.36E−01 | 1.06E+00 | 1 | Unchanged Low | |
| O00322 | M09cd7 | uroplakin 1A | 223.5286 | 295 | 262.0828 | 260.1175 | 2.30E−01 | 3.99E−01 | 1 | Unchanged Low | |
| P78368 | I09ab4 | casein kinase 1: gamma 2 | 211.1048 | 272 | 262.1137 | 248.257 | 3.12E−01 | 3.63E−01 | 1 | Unchanged Low | |
| Q01081 | I22cd4 | U2(RNU2) small nuclear RNA au | 138.1533 | 91.3 | 262.1811 | 163.878 | 9.24E−01 | −5.98E−01 | 1 | Unchanged Low | |
| O43193 | J15ab5 | G protein-coupled receptor 38 | 69.57316 | 206 | 262.301 | 179.1305 | 1.91E+00 | 1.56E+00 | 1 | Unchanged Low | |
| Q9Y3B0 | M03ef2 | CGI-105 protein | 174.5026 | 177 | 262.5458 | 204.6742 | 5.89E−01 | 2.03E−02 | 1 | Unchanged Low | |
| Q9NV80 | H05gh3 | WD repeat domain 12 | 178.5139 | 279 | 262.6286 | 239.9609 | 5.57E−01 | 6.43E−01 | 1 | Unchanged Low | |
| Q9Y473 | O18cd4 | zinc finger protein 175 | 141.3794 | 275 | 263.1099 | 226.3406 | 8.96E−01 | 9.57E−01 | 1 | Unchanged Low | |
| Q9Y548 | L20gh2 | hypothetical protein DJ167A19.1 | 233.5442 | 240 | 263.4412 | 245.7773 | 1.74E−01 | 4.14E−02 | 1 | Unchanged Low | |
| Q13952 | N15ef6 | nuclear transcription factor Y: ga | 241.7469 | 354 | 263.6055 | 286.4173 | 1.25E−01 | 5.50E−01 | 1 | Unchanged Low | |
| P18615 | K17cd3 | RD RNA-binding protein | 237.0199 | 242 | 263.7642 | 247.7362 | 1.54E−01 | 3.25E−02 | 1 | Unchanged Low | |
| P33241 | B23ef1 | lymphocyte-specific protein 1 | 94.80267 | 131 | 264.242 | 163.4955 | 1.48E+00 | 4.71E−01 | 1 | Unchanged Low | |
| O15116 | C01ef4 | Lsm1 protein | 344.8146 | 479 | 264.6979 | 362.8572 | −3.81E−01 | 4.74E−01 | 1 | Unchanged Low | |
| P50897 | A11ab8 | palmitoyl-protein thioesterase 1 ( | 126.6311 | 142 | 264.8236 | 177.9418 | 1.06E+00 | 1.69E−01 | 1 | Unchanged Low | |
| | F20gh8 | MADP-1 protein | 134.6124 | 228 | 264.9564 | 209.3298 | 9.77E−01 | 7.63E−01 | 1 | Unchanged Low | |
| Q9Y3A5 | G12ef2 | CGI-97 protein | 469.4567 | 544 | 265.0744 | 426.2946 | −8.25E−01 | 2.14E−01 | 1 | Unchanged Low | |
| Q9BVT1 | G03gh6 | leukocyte receptor cluster (LRC) | 282.5457 | 328 | 265.5443 | 291.9599 | −8.95E−02 | 2.14E−01 | 1 | Unchanged Low | |
| Q9UM44 | E10cd7 | HERV-N LTR-associating 2 | 180.7238 | 119 | 265.5625 | 188.5666 | 5.55E−01 | −5.98E−01 | 1 | Unchanged Low | |
| Q9UGC7 | A05gh3 | similar to prokaryotic-type class I | 289.9756 | 362 | 265.7778 | 306.0797 | −1.26E−01 | 3.22E−01 | 1 | Unchanged Low | |
| P49146 | G01ab5 | neuropeptide Y receptor Y2 | 222.0084 | 57.8 | 265.9266 | 181.9126 | 2.60E−01 | −1.94E+00 | 1 | Unchanged Low | |
| P54687 | I03ab3 | branched chain aminotransferase | 103.4218 | 93.8 | 266.0547 | 154.4299 | 1.36E+00 | −1.41E−01 | 1 | Unchanged Low | |
| O60884 | O09cd6 | DnaJ (Hsp40) homolog: subfamil | 330.3137 | 367 | 266.0819 | 321.0577 | −3.12E−01 | 1.51E−01 | 1 | Unchanged Low | |
| Q9NVC6 | J01cd1 | cofactor required for Sp1 transcri | 183.0562 | 150 | 266.1556 | 199.8392 | 5.40E−01 | −2.84E−01 | 1 | Unchanged Low | |
| O43399 | G06cd2 | tumor protein D52-like 2 | 283.4636 | 348 | 266.2168 | 299.1558 | −9.06E−02 | 2.95E−01 | 1 | Unchanged Low | |
| O75880 | K22cd2 | SCO cytochrome osidase deficie | 156.7184 | 117 | 266.2419 | 179.8238 | 7.65E−01 | −4.28E−01 | 1 | Unchanged Low | |
| P49406 | F01gh1 | mitochondrial ribosomal protein | 577.0339 | 353 | 266.3918 | 398.6973 | −1.12E+00 | −7.10E−01 | 1 | Unchanged Low | |
| Q9NYK5 | E15gh2 | mitochondrial ribosomal protein | 473.2712 | 449 | 266.7642 | 396.3322 | −8.27E−01 | −7.61E−02 | 1 | Unchanged Low | |
| O15428 | P18cd2 | protein (peptidyl-prolyl cis/trans i | 185.1313 | 249 | 267.1449 | 233.6377 | 5.29E−01 | 4.25E−01 | 1 | Unchanged Low | |
| O14842 | K04ab5 | G protein-coupled receptor 40 | 222.6002 | 170 | 267.4241 | 219.8563 | 2.65E−01 | −3.93E−01 | 1 | Unchanged Low | |
| O15262 | I06cd6 | ring finger protein 3 | 243.845 | 401 | 267.4275 | 304.1323 | 1.33E−01 | 7.18E−01 | 1 | Unchanged Low | |
| Q9NP61 | J01ef3 | ADP-ribosytation factor GTPase | 344.6989 | 175 | 267.443 | 262.4544 | −3.66E−01 | −9.78E−01 | 1 | Unchanged Low | |
| O15491 | P22cd5 | WD-repeat protein | 254.6166 | 355 | 267.6951 | 292.4461 | 7.23E−02 | 4.80E−01 | 1 | Unchanged Low | |
| O75390 | P01ab5 | citrate synthase | 656.561 | 312 | 268.0488 | 412.1526 | −1.29E+00 | −1.07E+00 | 1 | Unchanged Low | |
| P57088 | I12gh3 | hypothetical protein FLJ10525 | 377.5067 | 349 | 268.1833 | 331.588 | −4.93E−01 | −1.13E−01 | 1 | Unchanged Low | |
| Q9NRY6 | K06gh4 | phospholipid scramblase 3 | 272.0774 | 334 | 268.2248 | 291.4542 | −2.06E−02 | 2.96E−01 | 1 | Unchanged Low | |
| O15403 | E05cd5 | solute carrier family 16 (monocar | 384.2992 | 73.5 | 268.3423 | 242.0422 | −5.18E−01 | −2.39E+00 | 1 | Unchanged Low | |
| Q9BPZ5 | K09gh6 | hypothetical protein MGC3123 | 127.3745 | 286 | 268.7928 | 227.363 | 1.08E+00 | 1.17E+00 | 1 | Unchanged Low | |
| P10301 | P01cd2 | related RAS viral (r-ras) oncoge | 314.4978 | 204 | 268.8367 | 262.4726 | −2.26E−01 | −6.24E−01 | 1 | Unchanged Low | |
| | A20gh7 | HLA-B associated transcript 4 | 168.0802 | 234 | 269.0656 | 223.8166 | 6.79E−01 | 4.79E−01 | 1 | Unchanged Low | |
| Q9P018 | A08ef8 | HSPC142 protein | 196.1899 | 346 | 289.5447 | 270.6564 | 4.58E−01 | 8.20E−01 | 1 | Unchanged Low | |
| Q9Y4H2 | L10cd3 | insulin receptor substrate 2 | 297.029 | 329 | 270.4962 | 298.8227 | −1.35E−01 | 1.47E−01 | 1 | Unchanged Low | |
| Q9UJX3 | F04ef2 | anaphase-promoting complex su | 210.2906 | 204 | 270.642 | 228.4449 | 3.84E−01 | −4.10E−02 | 1 | Unchanged Low | |
| Q9H5X0 | O01gh6 | hypothetical protein MGC2941 | 333.5834 | 308 | 270.759 | 304.1289 | −3.01E−01 | −1.15E−01 | 1 | Unchanged Low | |
| Q92820 | H23cd4 | gamma-glutamyl hydrolase (conj | 78.13783 | 101 | 271.925 | 150.359 | 1.80E+00 | 3.70E−01 | 1 | Unchanged Low | |
| Q9UKK3 | K15ab2 | ADP-ribosyltransferase (NAD+: p | 337.297 | 450 | 271.9944 | 352.9975 | −3.10E−01 | 4.15E−01 | 1 | Unchanged Low | |
| Q9NWC8 | N13gh2 | hypothetical protein FLJ10120 | 315.7654 | 301 | 272.0013 | 296.1206 | −2.15E−01 | −7.10E−02 | 1 | Unchanged Low | |
| O14965 | I07cd2 | serine/threonine kinase 6 | 139.2727 | 101 | 272.158 | 170.6789 | 9.67E−01 | −4.69E−01 | 1 | Unchanged Low | |
| P50613 | B11ef5 | cyclin-dependent kinase 7 (MO15 | 286.8535 | 423 | 272.2018 | 327.4234 | −7.56E−02 | 5.61E−01 | 1 | Unchanged Low | |
| Q9H1C4 | H08gh7 | unc-93 homolog B1 (C. elegans) | 348.8713 | 501 | 272.3385 | 374.1085 | −3.57E−01 | 5.22E−01 | 1 | Unchanged Low | |
| P21851 | E13ab4 | adaptor-related protein complex: | 338.7753 | 373 | 272.4817 | 328.1045 | −3.14E−01 | 1.39E−01 | 1 | Unchanged Low | |
| Q9UGJ0 | B14ef2 | protein kinase; AMP-activated; g | 425.3015 | 543 | 272.5312 | 413.4782 | −6.42E−01 | 3.51E−01 | 1 | Unchanged Low | |
| P04035 | D17ab6 | 3-hydroxy-3-methylglutaryl-Coen | 242.9132 | 169 | 273.2324 | 228.5315 | 1.70E−01 | −5.20E−01 | 1 | Unchanged Low | |
| Q9UKY7 | K03gh3 | hypothetical protein H41 | 643.0085 | 272 | 273.736 | 396.2551 | −1.23E+00 | −1.24E+00 | 1 | Unchanged Low | |
| O95478 | B18gh1 | hypothetical protein YR-29 | 248.2059 | 221 | 273.8047 | 247.5607 | 1.42E−01 | −1.70E−01 | 1 | Unchanged Low | |
| Q9Y3W9 | B16ef8 | sentrin/SUMO-specific protease | 220.4064 | 333 | 274.1753 | 275.7642 | 3.15E−01 | 5.94E−01 | 1 | Unchanged Low | |
| Q9Y325 | A20ef2 | CGI-36 protein | 346.618 | 338 | 274.2434 | 319.4542 | −3.38E−01 | −3.85E−02 | 1 | Unchanged Low | |
| Q92854 | B09cd6 | sema domain; immunoglobulin d | 342.6474 | 183 | 274.4146 | 266.8232 | −3.20E−01 | −9.02E−01 | 1 | Unchanged Low | |
| P16106 | O08gh6 | H3 histone family; member C | 105.4744 | 221 | 274.8424 | 200.5879 | 1.38E+00 | 1.07E+00 | 1 | Unchanged Low | |
| O43143 | M05ab4 | DEAD/H (Asp-Glu-Ala-Asp/His) t | 412.7015 | 349 | 274.9731 | 345.6042 | −5.86E−01 | −2.41E−01 | 1 | Unchanged Low | |
| Q13547 | L12ef6 | histone deacetylase 1 | 375.5725 | 372 | 275.2293 | 340.9663 | −4.48E−01 | −1.34E−02 | 1 | Unchanged Low | |
| Q9H6F9 | D11gh5 | hypothetical protein FLJ22313 | 306.5811 | 290 | 275.269 | 290.6336 | −1.55E−01 | −8.00E−02 | 1 | Unchanged Low | |
| Q9NX18 | M08gh2 | hypothetical protein FLJ20487 | 311.71 | 394 | 275.6418 | 327.1007 | −1.77E−01 | 3.38E−01 | 1 | Unchanged Low | |
| O94764 | P03cd5 | RAN binding protein 9 | 256.3223 | 268 | 275.8144 | 266.5947 | 1.06E−01 | 6.24E−02 | 1 | Unchanged Low | |
| P82909 | N18gh8 | mitochondrial ribosomal protein S | 261.4618 | 343 | 275.8463 | 293.6016 | 7.73E−02 | 3.94E−01 | 1 | Unchanged Low | |
| Q9UI07 | D18ef8 | likely ortholog of mouse heat sho | 199.8914 | 199 | 276.061 | 225.0966 | 4.66E−01 | −4.00E−03 | 1 | Unchanged Low | |
| P20337 | M01ef5 | RAB3B; member RAS oncogene | 214.8304 | 126 | 276.148 | 205.8132 | 3.62E−01 | −7.65E−01 | 1 | Unchanged Low | |
| Q07960 | B22ab2 | Rho GTPase activating protein 1 | 219.98 | 218 | 276.184 | 237.9303 | 3.28E−01 | −1.55E−02 | 1 | Unchanged Low | |
| P49448 | P03cd7 | Glutamate dehydrogenase-2 | 344.3683 | 229 | 276.2751 | 283.2713 | −3.18E−01 | −5.88E−01 | 1 | Unchanged Low | |
| Q9NX48 | K08gh2 | hypothetical protein FLJ20442 | 493.7568 | 601 | 276.8343 | 457.1855 | −8.35E−01 | 2.83E−01 | 1 | Unchanged Low | |
| Q9NV56 | J03gh3 | chromosome 20 open reading fra | 138.9543 | 104 | 277.4945 | 173.6478 | 9.98E−01 | −4.11E−01 | 1 | Unchanged Low | |
| O00154 | L21cd7 | brain acyl-CoA hydrolase | 173.5669 | 189 | 277.5737 | 213.4547 | 6.77E−01 | 1.25E−01 | 1 | Unchanged Low | |
| Q9UI70 | B04ef1 | nucleolar protein ANKT | 307.7657 | 349 | 277.6076 | 311.4328 | −1.49E−01 | 1.81E−01 | 1 | Unchanged Low | |
| P26045 | N17ab8 | protein tyrosine phosphatase; no | 412.292 | 315 | 277.6603 | 334.8518 | −5.70E−01 | −3.90E−01 | 1 | Unchanged Low | |
| Q9Y2A8 | C24ef3 | CD209 antigen-like | 72.21222 | 91.3 | 277.8103 | 147.0989 | 1.94E+00 | 3.38E−01 | 1 | Unchanged Low | |
| Q9BZ89 | F08gh7 | phospholipase A2; group XII | 175.384 | 216 | 277.9804 | 223.0558 | 6.84E−01 | 2.99E−01 | 1 | Unchanged Low | |
| P35052 | J24ab4 | glypican 1 | 214.4355 | 173 | 278.2085 | 221.7567 | 3.76E−01 | −3.13E−01 | 1 | Unchanged Low | |
| Q9H0S9 | K21gh7 | nuclear receptor binding factor-2 | 80.19669 | 155 | 278.4321 | 171.2074 | 1.80E+00 | 9.51E−01 | 1 | Unchanged Low | |
| Q15654 | I07ef7 | thyroid hormone receptor interac | 284.295 | 238 | 278.8016 | 260.4802 | 7.71E−02 | −1.49E−01 | 1 | Unchanged Low | |
| P51948 | I09ab7 | menage a trois 1 (CAK assembly | 239.1769 | 272 | 278.9368 | 263.2799 | 2.22E−01 | 1.84E−01 | 1 | Unchanged Low | |
| Q13133 | B20cd5 | nuclear receptor subfamily 1: gro | 276.5554 | 260 | 279.1408 | 272.0232 | 1.34E−02 | −8.70E−02 | 1 | Unchanged Low | |
| Q9P0J9 | H22gh3 | goliath protein | 337.3229 | 215 | 279.5646 | 277.1331 | −2.71E−01 | −6.53E−01 | 1 | Unchanged Low | |
| Q15546 | G23cd8 | monocyte to rnacrophage differn | 136.5038 | 94 | 279.6415 | 170.0401 | 1.03E+00 | −5.39E−01 | 1 | Unchanged Low | |
| P28325 | E09ab5 | cystatin D | 371.6792 | 309 | 279.7092 | 320.2206 | −4.10E−01 | −2.65E−01 | 1 | Unchanged Low | |
| O94985 | O04ef8 | calsyntenin 1 | 137.0471 | 205 | 279.921 | 207.2587 | 1.03E+00 | 5.80E−01 | 1 | Unchanged Low | |
| P48634 | M03cd4 | HLA-B associated transcript 2 | 266.8369 | 179 | 280.5497 | 241.9692 | 7.23E−02 | −5.80E−01 | 1 | Unchanged Low | |
| Q9BRL6 | O15gh1 | Splicing factor: arginine/serine-ri | 220.3804 | 304 | 280.5881 | 268.4079 | 3.48E−01 | 4.65E−01 | 1 | Unchanged Low | |
| O94851 | E06gh1 | KIAA0750 gene product | 108.6672 | 172 | 280.9738 | 187.2889 | 1.37E+00 | 6.64E−01 | 1 | Unchanged Low | |
| Q9NW56 | E20gh3 | hypothetical protein FLJ10305 | 240.4538 | 314 | 281.002 | 278.4551 | 2.25E−01 | 3.85E−01 | 1 | Unchanged Low | |
| Q9H6X6 | P19gh5 | CasL interacting molecule | 183.3739 | 302 | 281.185 | 255.4932 | 6.17E−01 | 7.19E−01 | 1 | Unchanged Low | |
| P41217 | N13ab7 | antigen idenfified by monoclonal | 325.1642 | 166 | 281.3505 | 257.5072 | −2.09E−01 | −9.70E−01 | 1 | Unchanged Low | |
| P31512 | P04ab4 | flavin containing monooxygenase | 192.4074 | 276 | 281.6555 | 249.9252 | 5.50E−01 | 5.19E−01 | 1 | Unchanged Low | |
| Q15425 | H05cd2 | SA hypertension-associated horr | 186.2788 | 151 | 281.9205 | 206.4709 | 5.98E−01 | −3.01E−01 | 1 | Unchanged Low | |
| P40306 | L05ab8 | proteasome (prosome; macropai | 408.3043 | 513 | 281.9429 | 400.975 | −5.34E−01 | 3.28E−01 | 1 | Unchanged Low | |
| Q9NWZ8 | O02gh2 | hypothetical protein FLJ20514 | 287.569 | 146 | 281.9498 | 238.3826 | −2.85E−02 | −9.82E−01 | 1 | Unchanged Low | |
| P57073 | E22ef8 | SPY (sex determining region Y)-I | 202.5886 | 268 | 281.9705 | 251.002 | 4.77E−01 | 4.06E−01 | 1 | Unchanged Low | |
| Q9Y450 | H22cd6 | HBS1-like (S. cerevisiae) | 230.8266 | 312 | 282.4288 | 274.9973 | 2.91E−01 | 4.34E−01 | 1 | Unchanged Low | |
| P04062 | O08ab3 | glucosidase; beta; acid (includes | 295.5318 | 389 | 282.6453 | 322.2918 | −6.43E−02 | 3.95E−01 | 1 | Unchanged Low | |
| O75065 | E22gh1 | phosphodiesterase 4D interacting | 168.0201 | 154 | 282.6456 | 201.6823 | 7.50E−01 | −1.22E−01 | 1 | Unchanged Low | |
| Q9ULX2 | F09ef3 | NIMA(never in mitosis gene a)-r | 288.5578 | 208 | 283.1715 | 259.9871 | −2.72E−02 | −4.71E−01 | 1 | Unchanged Low | |
| Q99871 | C06ef4 | three prime repair exonuclease 2 | 205.6083 | 225 | 283.2504 | 237.9086 | 4.62E−01 | 1.29E−01 | 1 | Unchanged Low | |
| Q93065 | I15cd6 | PERB11 family member in MHC | 295.7132 | 457 | 283.4269 | 345.3524 | −6.12E−02 | 6.28E−01 | 1 | Unchanged Low | |
| O43837 | B12ab7 | isocitrate dehydrogenase 3 (NAD | 348.9961 | 298 | 283.6422 | 310.0846 | −2.99E−01 | −2.30E−01 | 1 | Unchanged Low | |
| Q9H9L3 | L06gh7 | hypothetical protein FLJ12671 | 209.5906 | 279 | 283.6912 | 257.4051 | 4.37E−01 | 4.12E−01 | 1 | Unchanged Low | |
| Q13740 | A18ab2 | activated leucocyte cell adhesion | 231.4555 | 253 | 283.7582 | 256.0277 | 2.94E−01 | 1.28E−01 | 1 | Unchanged Low | |
| Q12888 | L09cd2 | tumor protein p53 binding protein | 132.9276 | 223 | 283.8572 | 213.3434 | 1.09E+00 | 7.48E−01 | 1 | Unchanged Low | |
| P78318 | J13ab6 | immunoglobulin (CD79A) binding | 423.8715 | 512 | 283.9511 | 406.4772 | −5.78E−01 | 2.71E−01 | 1 | Unchanged Low | |
| P48436 | K24cd1 | SRY (sex determining region Y)-I | 345.2087 | 197 | 284.0786 | 275.3736 | −2.81E−01 | −8.10E−01 | 1 | Unchanged Low | |
| P18065 | B19ef1 | insulin-like growth factor binding | 286.6516 | 283 | 284.2331 | 284.4952 | −1.22E−02 | −2.05E−02 | 1 | Unchanged Low | |
| Q9ULW6 | F22cd8 | nucleosome assembly protein 1-I | 223.0694 | 148 | 284.717 | 218.587 | 3.52E−01 | −5.92E−01 | 1 | Unchanged Low | |
| O95154 | N03cd7 | aldo-keto reductase family 7; mei | 99.9079 | 298 | 284.8919 | 294.419 | −7.41E−02 | −7.00E−03 | 1 | Unchanged Low | |
| O95169 | P14ab8 | NADH dehydrogenase (ubiquinoi | 374.2624 | 199 | 285.0096 | 286.0758 | −3.93E−01 | −9.12E−01 | 1 | Unchanged Low | |
| Q99982 | C24ef4 | XIAP associated factor-1 | 63.10609 | 116 | 285.0252 | 154.8111 | 2.18E+00 | 8.82E−01 | 1 | Unchanged Low | |
| Q99470 | F14cd2 | stromal cell-derived factor 2 | 230.3684 | 316 | 285.1394 | 277.1416 | 3.08E−01 | 4.56E−01 | 1 | Unchanged Low | |
| P16104 | N19ab5 | H2A histone family: member X | 234.8351 | 195 | 285.2056 | 238.3532 | 2.80E−01 | −2.68E−01 | 1 | Unchanged Low | |
| Q9UG25 | B04ef8 | SOCS box-containing WD protei | 213.293 | 398 | 285.5392 | 298.9147 | 4.21E−01 | 9.00E−01 | 1 | Unchanged Low | |
| Q13158 | C21cd4 | Fas (TNFRSF6)-associated via d | 263.0157 | 281 | 285.9553 | 276.784 | 1.21E−01 | 9.74E−02 | 1 | Unchanged Low | |
| Q9NW32 | D10gh2 | hypothetical protein FLJ10346 | 235.0532 | 386 | 286.3524 | 302.6214 | 2.85E−01 | 7.17E−01 | 1 | Unchanged Low | |
| O95153 | M05cd5 | peripheral benzodiazepine recep | 155.1824 | 231 | 287.1271 | 224.5106 | 8.88E−01 | 5.75E−01 | 1 | Unchanged Low | |
| Q9BQ73 | H06gh7 | hypothetical protein FKSG28 | 212.0736 | 297 | 287.201 | 265.3817 | 4.37E−01 | 4.85E−01 | 1 | Unchanged Low | |
| Q15426 | C02ef7 | protein tyrosine phosphatase: re | 296.9619 | 441 | 287.2583 | 341.7927 | −4.79E−02 | 5.71E−01 | 1 | Unchanged Low | |
| Q92858 | L12ab2 | atonal homolog 1 (Drosophila) | 611.3779 | 278 | 287.2641 | 392.3029 | −1.09E+00 | −1.14E+00 | 1 | Unchanged Low | |
| Q16849 | P15ab8 | protein tyrosine phosphataso: re | 145.9896 | 55.6 | 287.3262 | 162.9842 | 9.77E−01 | −1.39E+00 | 1 | Unchanged Low | |
| P11279 | D07ab7 | lysosomal-associated membrane | 369.9925 | 310 | 287.3935 | 322.5735 | −3.64E−01 | −2.54E−01 | 1 | Unchanged Low | |
| Q96II1 | I16gh7 | hypothetical protein MGC14961 | 103.7251 | 80.7 | 287.4071 | 157.2667 | 1.47E+00 | −3.63E−01 | 1 | Unchanged Low | |
| P78504 | O20ab2 | jagged 1 (Alagille syndrome) | 255.0945 | 347 | 287.4633 | 296.4108 | 1.72E−01 | 4.43E−01 | 1 | Unchanged Low | |
| O75937 | G06ef3 | DnaJ (Hsp40) homolog; subfamil | 294.9992 | 264 | 287.503 | 282.0586 | −3.71E−02 | −1.62E−01 | 1 | Unchanged Low | |
| O60575 | O16ef4 | serine protease inhibitor; Kazal ty | 142.2326 | 152 | 287.9 | 194.1988 | 1.02E+00 | 1.00E−01 | 1 | Unchanged Low | |
| Q9NYP7 | K05gh5 | homolog of yeast long chain poly | 299.2029 | 187 | 288.1627 | 257.9759 | −5.42E−02 | −6.81E−01 | 1 | Unchanged Low | |
| O95436 | H01cd6 | solute carrier family 34 (sodium p | 238.6021 | 425 | 288.3247 | 317.3784 | 2.73E−01 | 8.34E−01 | 1 | Unchanged Low | |
| O95324 | B15ab2 | ATP-binding cassette; sub-family | 267.704 | 275 | 288.4558 | 277.0901 | 1.08E−01 | 3.94E−02 | 1 | Unchanged Low | |
| Q9UQF6 | B08ef3 | ribosomal protein L36 | 519.2003 | 425 | 288.5855 | 410.9115 | −8.47E−01 | −2.69E−01 | 1 | Unchanged Low | |
| P13010 | K12ef6 | X-ray repair complementing defe | 174.6996 | 256 | 288.9405 | 239.8403 | 7.26E−01 | 5.51E−01 | 1 | Unchanged Low | |
| Q9UJZ1 | P03cd8 | stomatin (EPB72)-like 2 | 490.6115 | 272 | 289.2003 | 350.5499 | −7.63E−01 | −8.52E−01 | 1 | Unchanged Low | |
| P50281 | I18ab7 | matrix metalloproteinase 14 (mer | 126.7204 | 196 | 289.8803 | 204.0657 | 1.19E+00 | 6.26E−01 | 1 | Unchanged Low | |
| P15927 | B08cd1 | replication protein A2 (32kD) | 100.0172 | 194 | 290.3306 | 194.7022 | 1.54E+00 | 9.54E−01 | 1 | Unchanged Low | |
| O75496 | B02ef1 | geminin | 466.4376 | 454 | 290.7216 | 403.7854 | −6.82E−01 | −3.84E−02 | 1 | Unchanged Low | |
| Q9P1E3 | C23gh4 | presenilins associated rhomboid | 232.5496 | 263 | 291.2445 | 262.3787 | 3.25E−01 | 1.79E−01 | 1 | Unchanged Low | |
| P40189 | B14ab6 | interleukin 6 signal transducer (g | 330.8834 | 315 | 291.3287 | 312.5572 | −1.84E−01 | −6.89E−02 | 1 | Unchanged Low | |
| Q9Y259 | H03ab4 | choline kinase-like | 328.1801 | 375 | 291.3288 | 331.557 | −1.72E−01 | 1.93E−01 | 1 | Unchanged Low | |
| O00633 | C12ef5 | phosphatase and tensin homolog | 199.7323 | 189 | 291.6118 | 226.7943 | 5.46E−01 | −7.94E−02 | 1 | Unchanged Low | |
| Q13432 | H13cd5 | unc-119 homolog (C. elegans) | 127.1818 | 268 | 291.793 | 229.0999 | 1.20E+00 | 1.08E+00 | 1 | Unchanged Low | |
| Q9Y677 | E03ef2 | COP9 constitutive photomorphog | 186.9367 | 204 | 291.9539 | 227.7917 | 6.43E−01 | 1.29E−01 | 1 | Unchanged Low | |
| Q9BW24 | D03ef8 | KIAA0676 protein | 170.3265 | 240 | 292.9836 | 234.5628 | 7.83E−01 | 4.97E−01 | 1 | Unchanged Low | |
| Q9NSD9 | B12cd5 | phenylalanyl-tRNA synthetase b | 226.6641 | 219 | 293.111 | 246.1288 | 3.71E−01 | −5.22E−02 | 1 | Unchanged Low | |
| Q9NWQ0 | C16gh3 | hypothetical protein FLJ20695 | 497.4612 | 656 | 293.1504 | 482.328 | −7.63E−01 | 4.00E−01 | 1 | Unchanged Low | |
| Q9NWT6 | C02gh3 | hypoxia-inducible factor 1; alpha | 521.7793 | 276 | 293.7543 | 363.8746 | −8.29E−01 | −9.18E−01 | 1 | Unchanged Low | |
| Q9UPN3 | J12ef7 | microtubule-actin crosslinking fa | 578.3578 | 563 | 294.1533 | 478.6344 | −9.75E−01 | −3.78E−02 | 1 | Unchanged Low | |
| Q96HW0 | A06gh7 | zinc finger protein 38 (KOX 25) | 126.7599 | 355 | 294.3016 | 258.7634 | 1.22E+00 | 1.49E+00 | 1 | Unchanged Low | |
| Q14257 | F18ab8 | reticulocalbin 2: EF-hand calcium | 237.969 | 217 | 295.1469 | 249.8542 | 3.11E−01 | −1.36E−01 | 1 | Unchanged Low | |
| Q9NRJ5 | J05gh4 | poly(A) polymerase beta (testis s | 379.2857 | 276 | 295.7341 | 316.91 | −3.59E−01 | −4.60E−01 | 1 | Unchanged Low | |
| P23280 | P02ab3 | carbonic anhydrase VI | 137.6155 | 102 | 296.1358 | 178.6789 | 1.11E+00 | −4.28E−01 | 1 | Unchanged Low | |
| Q14677 | I02gh1 | KIAA0171 gene product | 330.738 | 204 | 296.1674 | 277.0019 | −1.59E−01 | −6.96E−01 | 1 | Unchanged Low | |
| Q9Y394 | H17ef2 | CGI-86 protein | 471.0368 | 358 | 296.7589 | 375.1673 | −6.67E−01 | −3.97E−01 | 1 | Unchanged Low | |
| Q9UKX5 | L18cd7 | integrin: alpha 11 | 99.18133 | 126 | 297.0397 | 173.978 | 1.58E+00 | 3.42E−01 | 1 | Unchanged Low | |
| Q92539 | G02gh1 | lipin 2 | 260.7493 | 235 | 297.1151 | 264.3623 | 1.88E−01 | −1.49E−01 | 1 | Unchanged Low | |
| Q9NW01 | G10gh3 | hypothetical protein FLJ10402 | 165.6121 | 135 | 297.1276 | 199.1936 | 8.43E−01 | −2.97E−01 | 1 | Unchanged Low | |
| Q9NUW5 | N21gh3 | hypothetical protein FLJ11099 | 228.3252 | 247 | 297.5163 | 257.4882 | 3.82E−01 | 1.11E−01 | 1 | Unchanged Low | |
| Q9BYC4 | C13gh8 | mitochondrial ribosomal protein S | 478.9122 | 408 | 297.754 | 394.9285 | −6.86E−01 | −2.31E−01 | 1 | Unchanged Low | |
| P02545 | F01ab7 | lamin A/C | 707.7874 | 294 | 297.9921 | 433.306 | −1.25E+00 | −1.27E+00 | 1 | Unchanged Low | |
| O60585 | A17cd6 | serine/arginine repetitive matrix | 318.399 | 331 | 298.2057 | 315.9302 | −9.45E−02 | 5.68E−02 | 1 | Unchanged Low | |
| P23458 | L12ef5 | Janus kinase 1 (a protein tyrosin | 385.7348 | 441 | 298.2124 | 375.1408 | −3.71E−01 | 1.95E−01 | 1 | Unchanged Low | |
| Q9BY13 | O06gh8 | hook3 protein | 435.0319 | 471 | 298.2969 | 401.4116 | −5.44E−01 | 1.14E−01 | 1 | Unchanged Low | |
| Q9UBC2 | C05gh5 | epidermal growth factor receptor | 171.9695 | 271 | 298.6179 | 247.3586 | 7.96E−01 | 6.59E−01 | 1 | Unchanged Low | |
| O76022 | A14cd7 | E1B-55kDa-associated proteins | 256.9682 | 243 | 298.7615 | 266.0871 | 2.17E−01 | −8.34E−02 | 1 | Unchanged Low | |
| P33993 | K04ef6 | MCM7 minichromesome mainten | 239.0601 | 165 | 298.875 | 234.4011 | 3.22E−01 | −5.33E−01 | 1 | Unchanged Low | |
| Q92963 | F02cd2 | Ric-like; expressed in many tissu | 447.2008 | 499 | 299.9377 | 415.4004 | −5.76E−01 | 1.58E−01 | 1 | Unchanged Low | |
| Q9HAF1 | P09gh5 | hypothetical protein FLJ11730 | 270.4702 | 414 | 300.0397 | 328.0993 | 1.50E−01 | 6.13E−01 | 1 | Unchanged Low | |
| Q9NZ36 | P02gh4 | uncharacterized hypothalamus p | 505.7323 | 333 | 300.1219 | 379.485 | −7.53E−01 | −6.05E−01 | 1 | Unchanged Low | |
| Q9BQQ3 | C09gh8 | golgi phosphoprotein 5 | 208.3671 | 343 | 300.1696 | 283.9342 | 5.27E−01 | 7.20E−01 | 1 | Unchanged Low | |
| O95214 | P09ef3 | teptin receptor overlapping trans | 292.4076 | 419 | 300.357 | 337.2896 | 3.87E−02 | 5.19E−01 | 1 | Unchanged Low | |
| Q9Y294 | A21ef8 | DKFZP547E2110 protein | 166.5906 | 243 | 301.1573 | 237.0749 | 8.54E−01 | 5.47E−01 | 1 | Unchanged Low | |
| O95810 | O01cd4 | serum deprivation response (pho | 279.903 | 272 | 301.2133 | 284.3868 | 1.06E−01 | −4.11E−02 | 1 | Unchanged Low | |
| P30670 | L21ab4 | guanine nucleotide binding prote | 470.8713 | 466 | 301.8899 | 412.8188 | −6.41E−01 | −1.59E−02 | 1 | Unchanged Low | |
| P51946 | O14ef5 | Cyclin H | 180.4218 | 244 | 301.9337 | 242.2819 | 7.43E−01 | 4.38E−01 | 1 | Unchanged Low | |
| Q15290 | G18cd1 | retinoblastoma binding protein 6 | 298.4739 | 346 | 302.0906 | 315.4543 | 1.74E−02 | 2.12E−01 | 1 | Unchanged Low | |
| Q9NX11 | A03gh3 | hypothetical protein FLJ20498 | 165.3845 | 190 | 302.1175 | 219.2418 | 8.69E−01 | 2.02E−01 | 1 | Unchanged Low | |
| Q9P0S3 | A23ef2 | hypothetical protein L0C51240 | 187.2653 | 229 | 302.318 | 239.5316 | 6.91E−01 | 2.90E−01 | 1 | Unchanged Low | |
| P04554 | H11ab8 | protamine 2 | 393.1528 | 362 | 302.3989 | 352.4183 | −3.79E−01 | −1.20E−01 | 1 | Unchanged Low | |
| O15243 | O05ef4 | leptin receptor gene-related prot | 329.3033 | 391 | 302.5417 | 340.9139 | −1.22E−01 | 2.47E−01 | 1 | Unchanged Low | |
| O43717 | G03cd7 | tumor differentially expressed 1 | 284.4848 | 364 | 302.9444 | 317.004 | 9.07E−02 | 3.54E−01 | 1 | Unchanged Low | |
| O95876 | J10ef1 | hypothetical protein LOC51057 | 172.7128 | 226 | 302.9653 | 233.8891 | 8.11E−01 | 3.88E−01 | 1 | Unchanged Low | |
| Q9H8D5 | N17gh5 | tumor endothelial marker 6 | 247.105 | 190 | 303.4837 | 246.7925 | 2.96E−01 | −3.81E−01 | 1 | Unchanged Low | |
| Q9Y3B6 | D24ef1 | CGI-112 protein | 493.6755 | 288 | 303.7855 | 361.6747 | −7.01E−01 | −7.80E−01 | 1 | Unchanged Low | |
| Q9P0S7 | E13ef2 | hypothetical protein LOC51238 | 143.0704 | 139 | 303.896 | 195.3392 | 1.09E+00 | −4.11E−02 | 1 | Unchanged Low | |
| O60630 | D22ab4 | deltex homolog 1 (Drosophila) | 310.6617 | 221 | 303.9702 | 278.4627 | −3.14E−02 | −4.93E−01 | 1 | Unchanged Low | |
| O95285 | O07gh1 | macrophage erythroblast attache | 332.2343 | 413 | 304.0714 | 349.7511 | −1.28E−01 | 3.14E−01 | 1 | Unchanged Low | |
| Q08945 | G17cd2 | structure specific recognition pro | 335.05 | 229 | 304.6617 | 289.639 | −1.37E−01 | −5.48E−01 | 1 | Unchanged Low | |
| O43583 | D08cd3 | density-regulated protein | 203.0088 | 253 | 304.7034 | 253.5145 | 5.86E−01 | 3.17E−01 | 1 | Unchanged Low | |
| O95149 | D04cd5 | RNA; U transporter 1 | 186.979 | 254 | 304.889 | 248.5641 | 7.05E−01 | 4.41E−01 | 1 | Unchanged Low | |
| Q9HAS8 | N10gh4 | etaC homolog 2 (E. coli) | 243.3076 | 331 | 304.8937 | 293.0254 | 3.26E−01 | 4.44E−01 | 1 | Unchanged Low | |
| Q9BQ39 | A23gh6 | nucleolar protein GU2 | 234.3353 | 160 | 305.2734 | 233.2592 | 3.82E−01 | −5.49E−01 | 1 | Unchanged Low | |
| O15304 | C06ef6 | CD27-binding (Siva) protein | 88.75727 | 153 | 305.4708 | 182.3257 | 1.78E+00 | 7.83E−01 | 1 | Unchanged Low | |
| Q15394 | I08gh1 | basic leucine-zipper protein BZA | 347.7168 | 384 | 305.6252 | 345.6465 | −1.86E−01 | 1.42E−01 | 1 | Unchanged Low | |
| Q9NZA3 | N09ef1 | CDA14 | 235.1955 | 312 | 305.7623 | 284.2776 | 3.79E−01 | 4.07E−01 | 1 | Unchanged Low | |
| P26022 | P23ab8 | pentaxin-related gene: rapidly in | 97.61703 | 61.3 | 305.8598 | 154.9189 | 1.65E+00 | −6.72E−01 | 1 | Unchanged Low | |
| Q00536 | L24ef5 | PCTAIRE protein kinase 1 | 290.1914 | 359 | 306.0473 | 318.5577 | 7.68E−02 | 3.09E−01 | 1 | Unchanged Low | |
| Q9UMW5 | E19ef2 | cerebral cell adhesion molecule | 52.28353 | 234 | 306.6326 | 197.6582 | 2.55E+0O | 2.16E+00 | 1 | Unchanged Low | |
| O95353 | J17cd6 | Cdc42 effector protein 3 | 95.65314 | 208 | 306.7186 | 203.4709 | 1.68E+00 | 1.12E+00 | 1 | Unchanged Low | |
| O95808 | B03ef2 | arsenate resistance protein ARS: | 187.799 | 225 | 307.4311 | 240.0311 | 7.11E−01 | 2.60E−01 | 1 | Unchanged Low | |
| Q9BXJ8 | O17gh8 | transmembrane protein induced I | 411.8484 | 646 | 308.2116 | 455.5065 | −4.18E−01 | 6.50E−01 | 1 | Unchanged Low | |
| P36776 | A06cd5 | protease; serine; 15 | 390.4608 | 385 | 308.5755 | 354.6324 | −3.40E−01 | −9.78E−02 | 1 | Unchanged Low | |
| Q9BUR9 | M19gh6 | hypothetical protein MGC4614 | 250.3191 | 338 | 308.7431 | 299.1204 | 3.03E−01 | 4.35E−01 | 1 | Unchanged Low | |
| Q9Y3R1 | F23cd6 | Rab acceptor 1 (prenylated) | 158.9446 | 207 | 308.7948 | 224.7776 | 9.58E−01 | 3.78E−01 | 1 | Unchanged Low | |
| Q92997 | D03ef7 | dishevelled; dsh homolog 3 (Dros | 240.0941 | 305 | 309.2314 | 284.6254 | 3.65E−01 | 3.43E−01 | 1 | Unchanged Low | |
| O14682 | N17cd3 | ectodermal-neural cortex (with B | 326.0664 | 180 | 309.530 | 274.7193 | −7.50E−02 | −7.90E−01 | 1 | Unchanged Low | |
| Q9Y255 | I08cd8 | px19-like protein | 533.9688 | 455 | 309.7497 | 432.9739 | −7.86E−01 | −2.30E−01 | 1 | Unchanged Low | |
| Q16664 | O17ef4 | protein \A\″″ | 354.4762 | 286 | 310.2736 | 316.9703 | −1.92E−01 | −3.09E−01 | 1 | Unchanged Low | |
| P31943 | O04ab7 | heterogeneous nuclear ribonucle | 690.0051 | 250 | 310.5901 | 416.9005 | −1.15E+00 | −1.46E+00 | 1 | Unchanged Low | |
| P49848 | J09cd2 | TAF6 RNA polymerase II; TATA | 151.1422 | 133 | 310.9274 | 198.2917 | 1.04E+00 | −1.87E−01 | 1 | Unchanged Low | |
| Q04984 | P19ab6 | heat shock 10kD protein 1 (chap | 338.1568 | 327 | 311.1011 | 325.4422 | −1.20E−01 | −4.81E−02 | 1 | Unchanged Low | |
| P20719 | N08ef4 | homeo box A5 | 271.4135 | 326 | 311.2742 | 302.8287 | 1.98E−01 | 2.63E−01 | 1 | Unchanged Low | |
| Q9H5Y0 | D21gh7 | hypothetical protein FLJ22795 | 325.1674 | 305 | 311.3753 | 313.8457 | −6.25E−02 | −9.24E−02 | 1 | Unchanged Low | |
| P19021 | G15ab8 | peptidylglycine alpha-amidating | 219.5791 | 273 | 311.913 | 268.0943 | 5.06E−01 | 3.13E−01 | 1 | Unchanged Low | |
| Q92544 | B13gh1 | KIAA0255 gene product | 270.8313 | 307 | 312.0207 | 206.5662 | 2.04E−01 | 1.80E−01 | 1 | Unchanged Low | |
| Q12907 | G07cd7 | chromosome 5 open reading fra | 313.0938 | 222 | 312.5092 | 282.474 | −2.70E−03 | −4.97E−01 | 1 | Unchanged Low | |
| Q9BW91 | C03gh6 | nudix (nucleoside diphosphate li | 346.395 | 314 | 312.5575 | 324.483 | −1.48E−01 | −1.39E−01 | 1 | Unchanged Low | |
| P53365 | M23cd8 | partner of RAC1 (arfaptin 2) | 293.2566 | 340 | 312.9335 | 318.382 | 9.37E−02 | 2.51E−01 | 1 | Unchanged Low | |
| P35869 | O17ab2 | aryl hydrocarbon receptor | 249.5731 | 417 | 313.1153 | 326.5306 | 3.27E−01 | 7.40E−01 | 1 | Unchanged Low | |
| Q13145 | I19cd8 | putative tranamembrane protein | 490.8698 | 608 | 313.616 | 470.7589 | −6.46E−01 | 3.08E−01 | 1 | Unchanged Low | |
| O14747 | A18ef6 | death-associated protein 6 | 348.2382 | 450 | 313.7398 | 370.593 | −1.51E−01 | 3.69E−01 | 1 | Unchanged Low | |
| Q9GZT5 | H03gh7 | wingless-type MMTV integration | 362.0025 | 327 | 313.9764 | 334.369 | −2.05E−01 | −1.46E−01 | 1 | Unchanged Low | |
| P08174 | M19ef1 | decay accelerating factor for co | 705.5781 | 457 | 314.0939 | 402.3204 | −1.17E+00 | −6.26E−01 | 1 | Unchanged Low | |
| Q9H689 | K01gh4 | hypothetical protein RG083M05.: | 241.9641 | 291 | 314.1855 | 282.3328 | 3.77E−01 | 2.65E−01 | 1 | Unchanged Low | |
| Q9P0T7 | K05ef2 | hypothetical protein LOC51235 | 266.1575 | 324 | 314.3982 | 301.4061 | 2.40E−01 | 2.82E−01 | 1 | Unchanged Low | |
| Q9H2V7 | I12gh8 | spinster-like protein | 275.9977 | 394 | 314.4425 | 328.1261 | 1.88E−01 | 5.13E−01 | 1 | Unchanged Low | |
| Q9NWT0 | D23gh2 | hypothetical protein FLJ20625 | 420.628 | 488 | 314.6523 | 407.636 | −4.19E−01 | 2.13E−01 | 1 | Unchanged Low | |
| Q9Y6X2 | E22cd6 | protein inhibitor of activated STA | 149.0425 | 237 | 315.009 | 233.7882 | 1.08E+00 | 6.71E−01 | 1 | Unchanged Low | |
| Q9P1P1 | F10ef8 | clone FLB1727 (LOC51215) | 419.9659 | 330 | 315.0092 | 355.1102 | −4.15E−01 | −3.46E−01 | 1 | Unchanged Low | |
| P23919 | H04cd7 | deoxythymidylate kinase (thymid | 123.4582 | 84.1 | 315.562 | 174.3854 | 1.35E+00 | −5.53E−01 | 1 | Unchanged Low | |
| P37140 | D09ab8 | protein phosphatase 1; catalytic: | 355.0781 | 503 | 315.7751 | 391.3748 | −1.69E−01 | 5.03E−01 | 1 | Unchanged Low | |
| Q9NW54 | B22gh2 | homolog of rat nadrin | 432.3799 | 670 | 315.8314 | 472.8474 | −4.53E−01 | 6.33E−01 | 1 | Unchanged Low | |
| Q9UMS0 | C05ef4 | HIRA interacting protein 5 | 504.0153 | 382 | 315.8386 | 400.6081 | −6.74E−01 | 4.00E−01 | 1 | Unchanged Low | |
| P14207 | E23ef1 | folate receptor 2 (fetal) | 235.5291 | 154 | 316.0649 | 235.1893 | 4.24E−01 | −6.13E−01 | 1 | Unchanged Low | |
| Q9UHC7 | L05ef4 | makorin; ring finger protein; 1 | 243.752 | 243 | 316.3297 | 267.5313 | 3.76E−01 | −7.36E−03 | 1 | Unchanged Low | |
| O75844 | K23cd6 | zinc metalloproteinase (STE24 h | 338.221 | 174 | 316.3789 | 276.2357 | −9.63E−02 | −9.58E−01 | 1 | Unchanged Low | |
| | P18gh8 | LIM homeobox protein 4 | 87.11814 | 84.8 | 316.6454 | 162.8523 | 1.86E+00 | −3.90E−02 | 1 | Unchanged Low | |
| P16587 | P19ab2 | ADP-ribosylation factor 3 | 444.3724 | 348 | 317.1873 | 369.9292 | −4.86E−01 | −3.52E−01 | 1 | Unchanged Low | |
| Q9NVY2 | P18gh2 | hypothetical protein FLJ10439 | 420.4716 | 399 | 317.2936 | 378.7795 | −4.06E−01 | −7.72E−02 | 1 | Unchanged Low | |
| Q9Y311 | J09ef4 | F-box only protein 7 | 368.7457 | 422 | 317.6515 | 369.3121 | −2.15E−01 | 1.93E−01 | 1 | Unchanged Low | |
| Q9H320 | P21cd8 | variable charge; X chromosome | 147.0814 | 348 | 318.4769 | 271.2531 | 1.11E+00 | 1.24E+00 | 1 | Unchanged Low | |
| P49903 | J11ef4 | selenium donor protein | 329.031 | 446 | 318.9322 | 364.5082 | −4.50E−02 | 4.37E−01 | 1 | Unchanged Low | |
| O00506 | B05cd6 | serine/threonine kinase 25 (STE: | 273.696 | 275 | 319.3179 | 289.4017 | 2.22E−01 | 7.86E−03 | 1 | Unchanged Low | |
| O00273 | F14ab5 | DNA fragmentation factor; 45kD | 328.993 | 401 | 319.634 | 350.0198 | −4.16E−02 | 2.87E−01 | 1 | Unchanged Low | |
| Q08170 | H11cd2 | splicing factor; arginine/serine-ri | 231.1994 | 193 | 319.7733 | 247.9554 | 4.68E−01 | −2.61E−01 | 1 | Unchanged Low | |
| Q9Y388 | N15ef8 | small fragment nuclease | 120.8094 | 282 | 320.0578 | 240.8904 | 1.41E+00 | 1.22E+00 | 1 | Unchanged Low | |
| Q9P0N0 | E11eF2 | mitochondrial ribosomal protein | 377.6692 | 391 | 320.1382 | 362.9626 | −2.38E−01 | 5.03E−02 | 1 | Unchanged Low | |
| Q9BR13 | D11gh8 | hypothetical protein MGC11303 | 373.5695 | 391 | 320.4318 | 361.8895 | −2.21E−01 | 6.60E−02 | 1 | Unchanged Low | |
| Q9Y319 | D19ef2 | CGI-30 protein | 107.1253 | 123 | 320.4576 | 183.4471 | 1.58E+00 | 1.97E−01 | 1 | Unchanged Low | |
| Q16514 | M17gh1 | TAF 12 RNA polymerase II; TATA | 116.3618 | 304 | 320.8611 | 246.9777 | 1.46E+00 | 1.38E+00 | 1 | Unchanged Low | |
| P13489 | D13ef7 | ribonuclease/angiogenin inhibitor | 224.1663 | 232 | 321.0895 | 259.1076 | 5.18E−01 | 5.00E−02 | 1 | Unchanged Low | |
| Q99987 | P08ef5 | vaccinia related kinase 2 | 205.9202 | 194 | 321.2364 | 240.3908 | 6.42E−01 | −8.59E−02 | 1 | Unchanged Low | |
| P56181 | H21ef1 | NADH dehydrogenase (ubiquino | 435.0715 | 319 | 321.3696 | 358.4382 | −4.37E−01 | −4.48E−01 | 1 | Unchanged Low | |
| P51148 | N18ab8 | RAB5C; member RAS oncogene | 329.7976 | 358 | 321.5906 | 336.6203 | −3.64E−02 | 1.20E−01 | 1 | Unchanged Low | |
| Q9Hd34 | E12gh4 | CGI-203 protein | 257.866 | 249 | 322.345 | 276.3612 | 3.22E−01 | −5.12E−02 | 1 | Unchanged Low | |
| Q9HAN2 | F02gh6 | pumilio homolog 2 (Drosophila) | 291.9641 | 326 | 322.4096 | 313.4126 | 1.43E−01 | 1.58E−01 | 1 | Unchanged Low | |
| P08107 | K13ef5 | heat shock 70kD protein 1A | 535.4871 | 382 | 322.6223 | 413.3803 | −7.31E−01 | −4.87E−01 | 1 | Unchanged Low | |
| O60499 | N18cd3 | syntaxin 10 | 189.7411 | 304 | 322.7738 | 272.2579 | 7.86E−01 | 6.81E−01 | 1 | Unchanged Low | |
| Q9HBL3 | M19gh4 | N-terminal kinase-like | 165.2038 | 224 | 323.2472 | 237.4707 | 9.68E−01 | 4.38E−01 | 1 | Unchanged Low | |
| Q9BSJ8 | D22gh6 | KIAA0747 protein | 549.1645 | 575 | 323.5208 | 482.4222 | −7.63E−01 | 6.53E−02 | 1 | Unchanged Low | |
| O60838 | G09gh7 | SAC2 suppressor of actin mutati | 216.0571 | 254 | 324.4643 | 264.8237 | 5.87E−01 | 2.33E−01 | 1 | Unchanged Low | |
| Q9BTM6 | M11gh8 | hypothetical protein MGC3195 | 371.9412 | 334 | 324.5103 | 343.6429 | −1.97E−01 | −1.53E−01 | 1 | Unchanged Low | |
| Q9H4L0 | K06gh5 | leucine zipper protein FKSG14 | 47.34403 | 70.6 | 324.6661 | 147.5387 | 2.78E+00 | 5.77E−01 | 1 | Unchanged Low | |
| P50402 | I14ab3 | emerin (Emery-Dreifuss muscula | 321.3743 | 304 | 324.6751 | 316.6654 | 1.47E−02 | −8.04E−02 | 1 | Unchanged Low | |
| Q9NZP7 | G05ef3 | calcium binding protein Cab45 p | 336.1107 | 239 | 324.6956 | 299.844 | −4.98E−02 | −4.94E−01 | 1 | Unchanged Low | |
| P49116 | G22cd2 | nuclear receptor subfamily 2; gro | 295.9342 | 224 | 325.1253 | 281.6482 | 1.36E−01 | −4.03E−01 | 1 | Unchanged Low | |
| P11234 | D14ab8 | v-ral simian leukemia viral oncog | 565.699 | 444 | 325.5924 | 444.9874 | −7.97E−01 | −3.51E−01 | 1 | Unchanged Low | |
| P15408 | D21ab5 | FOS-like antigen 2 | 265.6989 | 437 | 325.8448 | 342.8236 | 2.94E−01 | 7.18E−01 | 1 | Unchanged Low | |
| Q9P1Q8 | N05cd8 | RAB3A interacting protein (rabin; | 357.8493 | 253 | 326.2784 | 312.2142 | −1.33E−01 | −5.03E−01 | 1 | Unchanged Low | |
| P39060 | P16ef6 | collagen; type XVIII; alpha 1 | 151.6584 | 212 | 326.8288 | 230.0488 | 1.11E+00 | 4.81E−01 | 1 | Unchanged Low | |
| Q9Y2N3 | N05gh1 | KIAA0618 gene product | 378.8717 | 252 | 326.8347 | 319.0933 | −2.13E−01 | −5.91E−01 | 1 | Unchanged Low | |
| P02749 | M22ab2 | apolipoprotein H (beta-2-glycopr | 408.7454 | 226 | 327.1228 | 320.758 | −3.21E−01 | −8.52E−01 | 1 | Unchanged Low | |
| Q9P189 | L20gh3 | hypothetical protein PRO1855 | 392.3328 | 358 | 327.1908 | 359.0228 | −2.62E−01 | −1.34E−01 | 1 | Unchanged Low | |
| O43822 | B19ab4 | chromosome 21 open reading fra | 249.9987 | 297 | 327.8679 | 291.7148 | 3.91E−01 | 2.50E−01 | 1 | Unchanged Low | |
| Q9UNZ2 | N15ef2 | likely ortholog of rat p47 | 413.5167 | 519 | 327.9429 | 420.06S4 | −3.35E−01 | 3.27E−01 | 1 | Unchanged Low | |
| Q9Y4G5 | B22cd8 | dynein; cytoplasmic; heavy polyp | 376.6624 | 426 | 327.9518 | 376.7971 | −2.00E−01 | 1.77E−01 | 1 | Unchanged Low | |
| Q13310 | C13cd4 | poly(A) binding protein; cytoplas | 316.8746 | 338 | 328.1257 | 327.507 | 5.03E−02 | 9.11E−02 | 1 | Unchanged Low | |
| P45974 | O14cd3 | ubiquitin specific protease 5 (isop | 268.1073 | 316 | 328.8257 | 304.4284 | 2.95E−01 | 2.39E−01 | 1 | Unchanged Low | |
| P55317 | A04ab7 | hepatocyte nuclear factor 3; alph | 273.7527 | 306 | 328.8562 | 302.6162 | 2.65E−01 | 1.60E−01 | 1 | Unchanged Low | |
| O43364 | H06cd8 | homeo box A2 | 511.053 | 468 | 329.1292 | 435.9554 | −6.35E−01 | −1.28E−01 | 1 | Unchanged Low | |
| P55345 | H15ab6 | HMT1 hnRNP methyltransferase | 364.8758 | 307 | 329.5023 | 333.8774 | −1.47E−01 | −2.48E−01 | 1 | Unchanged Low | |
| P48230 | O22cd2 | transmembrane 4 superfamily me | 565.1635 | 417 | 330.825 | 437.5376 | −7.73E−01 | −4.40E−01 | 1 | Unchanged Low | |
| Q9BUY3 | C21gh5 | sorting nexin 6 | 166.5144 | 297 | 330.8949 | 264.7192 | 9.91E−01 | 8.34E−01 | 1 | Unchanged Low | |
| O00115 | B01ef6 | deoxynbonuclease II: lysosomal | 275.2503 | 372 | 331.0554 | 326.0947 | 2.66E−01 | 4.34E−01 | 1 | Unchanged Low | |
| O43318 | M11cd2 | mitogen-activated protein kinase | 186.7739 | 247 | 331.1308 | 254.8269 | 8.26E−01 | 4.01E−01 | 1 | Unchanged Low | |
| O95572 | B08gh1 | mitofusin 2 | 252.7846 | 277 | 331.4516 | 287.175 | 3.91E−01 | 1.33E−01 | 1 | Unchanged Low | |
| P35080 | K12ab8 | profilin 2 | 111.4363 | 85.9 | 332.2299 | 176.518 | 1.58E+00 | −3.76E−01 | 1 | Unchanged Low | |
| Q9NW29 | E24gh3 | hypothetical protein FLJ10350 | 219.5611 | 173 | 332.8499 | 241.9521 | 6.00E−01 | −3.40E−01 | 1 | Unchanged Low | |
| Q9NZE9 | O07ef2 | BRAF35/HDAC2 complex (80kD | 219.3916 | 348 | 332.8813 | 299.9861 | 6.01E−01 | 6.64E−01 | 1 | Unchanged Low | |
| Q9BYV2 | E05gh8 | ring finger protein 30 | 500.2246 | 338 | 333.9675 | 390.6938 | −5.83E−01 | −5.66E−01 | 1 | Unchanged Low | |
| Q9UJX4 | F02ef2 | anaphase promoting complex sul | 237.1218 | 266 | 334.5884 | 280.1684 | 4.97E−01 | 1.81E−01 | 1 | Unchanged Low | |
| O00302 | B19cd6 | calcium homeostasis endoplasmi | 327.1489 | 382 | 334.7371 | 347.7981 | 3.31E−02 | 2.22E−01 | 1 | Unchanged Low | |
| Q92886 | K23cd1 | neurogenin 1 | 290.2357 | 283 | 335.1054 | 302.7027 | 2.07E−01 | −3.76E−02 | 1 | Unchanged Low | |
| O14929 | P07cd3 | histone acetyltransferase 1 | 344.9153 | 130 | 335.4301 | 270.1111 | −4.02E−02 | −1.41E+00 | 1 | Unchanged Low | |
| Q9NVK7 | M18gh3 | chromosome 19 open reading fra | 281.2043 | 287 | 335.4383 | 301.1995 | 2.54E−01 | 2.92E−02 | 1 | Unchanged Low | |
| Q9UBN0 | P09ef4 | proline rich 3 | 309.9093 | 344 | 335.4526 | 329.7234 | 1.14E−01 | 1.50E−01 | 1 | Unchanged Low | |
| O43611 | E14cd3 | cytoplasmic linker 2 | 180.9108 | 220 | 336.1459 | 245.5832 | 8.94E−01 | 2.80E−01 | 1 | Unchanged Low | |
| O94777 | G23cd4 | dolichyl-phosphate mannosyltran | 294.1356 | 244 | 336.7183 | 291.5641 | 1.95E−01 | −2.71E−01 | 1 | Unchanged Low | |
| Q14669 | K05gh1 | thyroid hormone receptor interac | 486.9829 | 397 | 337.0199 | 406.8782 | −5.31E−01 | −2.96E−01 | 1 | Unchanged Low | |
| Q9NUR0 | B20gh3 | hypothetical protein FLJ11196 | 63.09205 | 107 | 337.0769 | 168.9127 | 2.42E+00 | 7.56E−01 | 1 | Unchanged Low | |
| Q16822 | L08ab8 | phosphoenolpyruvate carboxykin | 262.4601 | 156 | 337.1655 | 251.8272 | 3.61E−01 | −7.52E−01 | 1 | Unchanged Low | |
| Q9Y640 | A14cd8 | stromal cell derived factor recept | 215.2348 | 220 | 337.2908 | 257.3848 | 6.48E−01 | 2.92E−02 | 1 | Unchanged Low | |
| P48595 | C01cd1 | serine (or cysteine) proteinase in | 62.04001 | 410 | 337.3872 | 269.7194 | 2.44E+00 | 2.72E+00 | 1 | Unchanged Low | |
| P26639 | M15cd2 | threonyl-tRNA synthetase | 355.1309 | 422 | 337.8401 | 371.5003 | −7.20E−02 | 2.47E−01 | 1 | Unchanged Low | |
| P29597 | O03cd3 | tyrosine kinase 2 | 209.3427 | 258 | 338.1471 | 268.5377 | 6.92E−01 | 3.02E−01 | 1 | Unchanged Low | |
| O60671 | B02ab8 | RAD1 homolog (S. pombe) | 188.2716 | 104 | 339.1461 | 210.3983 | 8.49E−01 | −8.59E−01 | 1 | Unchanged Low | |
| Q9NW97 | P17gh2 | hypothetical protein FLJ10199 | 262.3124 | 536 | 339.3086 | 379.0815 | 3.71E−01 | 1.03E+00 | 1 | Unchanged Low | |
| Q9P027 | O23ef8 | HSPC133 protein | 616.0461 | 363 | 339.3102 | 439.5495 | −8.60E−01 | −7.62E−01 | 1 | Unchanged Low | |
| O95772 | E20gh8 | hypothetical protein MGC3251 | 195.9084 | 227 | 339.3844 | 254.0574 | 7.93E−01 | 2.12E−01 | 1 | Unchanged Low | |
| Q9NR28 | N13gh4 | second mitochondria-derived act | 271.4457 | 543 | 339.8107 | 384.7351 | 3.24E−01 | 1.00E+00 | 1 | Unchanged Low | |
| P11413 | F15ab3 | glucose-6-phosphate dehydroge | 258.0938 | 135 | 340.0217 | 244.4906 | 3.98E−01 | −9.31E−01 | 1 | Unchanged Low | |
| Q99567 | A04ab8 | nucleoporin 88kD | 464.4826 | 320 | 340.5878 | 375.0149 | −4.48E−01 | −5.38E−01 | 1 | Unchanged Low | |
| Q9BZR9 | F22gh7 | ring finger protein 27 | 206.7918 | 398 | 340.7239 | 315.1764 | 7.20E−01 | 9.45E−01 | 1 | Unchanged Low | |
| O43632 | L22cd6 | gamma-tubulin complex protein 2 | 328.0052 | 322 | 341.4805 | 330.4468 | 5.81E−02 | −2.73E−02 | 1 | Unchanged Low | |
| Q9Y305 | I05cd8 | Mitochondrial Acyl-CoA Thioeste | 447.9229 | 475 | 341.5085 | 421.5064 | −3.91E−01 | 8.49E−02 | 1 | Unchanged Low | |
| P17096 | L14ef6 | high-mobility group (nonhistone | 393.4158 | 266 | 341.5301 | 333.78 | −2.04E−01 | −5.62E−01 | 1 | Unchanged Low | |
| P28677 | E10cd3 | visinin-like 1 | 235.283 | 241 | 341.6123 | 272.797 | 5.38E−01 | 3.76E−02 | 1 | Unchanged Low | |
| Q9UBX5 | K04cd6 | fibulin 5 | 79.52593 | 108 | 341.7638 | 176.4964 | 2.10E+00 | 4.44E−01 | 1 | Unchanged Low | |
| Q9Y304 | E12ef2 | ovarian cancer overexpressed 1 | 326.0044 | 238 | 341.8947 | 301.8585 | 6.87E−02 | −4.56E−01 | 1 | Unchanged Low | |
| O15379 | L16ef6 | histone deacetylase 3 | 198.0005 | 380 | 342.3529 | 306.8418 | 7.90E−01 | 9.41E−01 | 1 | Unchanged Low | |
| Q02790 | H16ab5 | FK506 binding protein 4 (59kD) | 323.5993 | 240 | 343.4254 | 302.2597 | 8.58E−02 | −4.33E−01 | 1 | Unchanged Low | |
| Q9H240 | K12ef5 | Notch homolog 2 (Drosophila) | 270.5904 | 566 | 343.4339 | 393.3189 | 3.44E−01 | 1.06E+00 | 1 | Unchanged Low | |
| O76095 | P22cd6 | jumping translocation breakpoint | 252.8696 | 227 | 343.482 | 274.4316 | 4.42E−01 | −1.56E−01 | 1 | Unchanged Low | |
| Q9Y5Q8 | P07cd7 | general transcription factor IIIC: p | 545.6403 | 324 | 343.9958 | 404.6602 | −6.66E−01 | −7.50E−01 | 1 | Unchanged Low | |
| Q9NNW5 | H06ef8 | WD repeat domain 6 | 254.1607 | 369 | 344.0253 | 322.3381 | 4.37E−01 | 5.37E−01 | 1 | Unchanged Low | |
| P01121 | C09ef6 | ras homolog gene family; membe | 241.7544 | 304 | 344.0732 | 296.4811 | 5.09E−01 | 3.29E−01 | 1 | Unchanged Low | |
| Q9H0V7 | K18gh8 | hypothetical protein DKFZp5640 | 568.248 | 304 | 344.2985 | 405.6684 | −7.23E−01 | −9.00E−01 | 1 | Unchanged Low | |
| O60664 | P02cd5 | cargo selection protein (mannose | 520.0919 | 232 | 344.5523 | 365.5782 | −5.94E−01 | −1.16E+00 | 1 | Unchanged Low | |
| P56554 | O15cd3 | ubiquitin-conjugating enzyme E2 | 374.4383 | 417 | 345.0269 | 378.959 | −1.18E−01 | 1.57E−01 | 1 | Unchanged Low | |
| O00330 | O06cd3 | Pyruvate dehydrogenase comple | 345.9372 | 430 | 345.2213 | 373.6892 | −2.99E−03 | 3.14E−01 | 1 | Unchanged Low | |
| O60784 | N17cd5 | target of myb1 (chicken) | 364.3098 | 317 | 345.2431 | 342.086 | −7.76E−02 | −2.02E−01 | 1 | Unchanged Low | |
| Q9NQZ0 | H03ef4 | CMP-N-acetylneuraminic acid sy | 788.5176 | 339 | 345.3498 | 491.0715 | −1.19E+00 | −1.22E+00 | 1 | Unchanged Low | |
| P52790 | A04ab5 | hexokinase 3 (white cell) | 260.6034 | 430 | 345.4621 | 345.2169 | 4.07E−01 | 7.21E−01 | 1 | Unchanged Low | |
| P52815 | B10cd1 | mitochondinal ribosomal protein | 234.3191 | 277 | 345.7776 | 285.7775 | 5.61E−01 | 2.43E−01 | 1 | Unchanged Low | |
| O14983 | O10ef1 | ATPase: Ca++ transporting: card | 167.4066 | 223 | 346.1734 | 245.4613 | 1.05E+00 | 4.12E−01 | 1 | Unchanged Low | |
| P04259 | D01ab7 | keratin 6B | 534.1837 | 405 | 347.6543 | 428.9832 | −6.20E−01 | −3.99E−01 | 1 | Unchanged Low | |
| P16455 | M04ef6 | O-6-methylguanine-DNA methylt | 299.0887 | 307 | 348.0545 | 318.0812 | 2.19E−01 | 3.81E−02 | 1 | Unchanged Low | |
| Q9Y5L9 | N04cd6 | Snf2-related CBP activator protei | 285.2875 | 187 | 348.0925 | 273.2945 | 2.87E−01 | −6.13E−01 | 1 | Unchanged Low | |
| P41214 | A16gh1 | ligatin | 237.0742 | 307 | 348.5538 | 297.4677 | 5.56E−01 | 3.72E−01 | 1 | Unchanged Low | |
| Q9H6U4 | P13gh5 | hypothetical protein FLJ21865 | 419.2747 | 175 | 348.8375 | 314.4039 | −2.65E−01 | −1.26E+00 | 1 | Unchanged Low | |
| Q15393 | L10ef7 | splicing factor 3b: subunit 3: 1301 | 242.1115 | 239 | 349.6571 | 277.0735 | 5.30E−01 | −1.59E−02 | I UnchangedLow | | |
| P55055 | C04gh1 | nuclear receptor subfamily 1; gro | 325.6291 | 368 | 349.8393 | 347.7942 | 1.03E−01 | 1.76E−01 | 1 | Unchanged Low | |
| P51805 | M03ef4 | SEX gene | 303.0664 | 383 | 350.6414 | 345.4093 | 2.10E−01 | 3.36E−01 | 1 | Unchanged Low | |
| O75815 | G23ab3 | breast cancer anti-estrogen resis | 249.1903 | 293 | 350.9571 | 297.768 | 4.94E−01 | 2.34E−01 | 1 | Unchanged Low | |
| Q9Y2Y9 | F19ef2 | Kruppel-like factor 13 | 241.1753 | 366 | 351.0501 | 319.2905 | 5.42E−01 | 6.00E−01 | 1 | Unchanged Low | |
| Q99633 | D04cd3 | pre-mRNA processing factor 18 | 405.046 | 346 | 351.3478 | 368.1886 | −2.05E−01 | −2.18E−01 | 1 | Unchanged Low | |
| P05155 | C08ab3 | serine (or cysteine) proteinase in | 303.3386 | 334 | 351.5074 | 329.7432 | 2.13E−01 | 1.41E−01 | 1 | Unchanged Low | |
| Q9UN50 | O16cd7 | EAP30 subunit of ELL complex | 321.9045 | 326 | 351.5535 | 333.1657 | 1.27E−01 | 1.84E−02 | 1 | Unchanged Low | |
| Q14011 | E11ab4 | cold inducible RNA binding prote | 292.5107 | 412 | 351.6685 | 352.0144 | 2.66E−01 | 4.94E−01 | 1 | Unchanged Low | |
| O75807 | M02ef3 | protein phosphatase 1; regulator | 371.7509 | 652 | 352.5048 | 458.6852 | −7.67E−02 | 8.10E−01 | 1 | Unchanged Low | |
| Q14457 | K19ab3 | beclin 1 (coiled-coil: myosin-like | 368.4142 | 678 | 352.9542 | 466.4393 | −6.18E−02 | 8.80E−01 | 1 | Unchanged Low | |
| Q02818 | J06ef6 | nucleobindin 1 | 330.8747 | 213 | 353.1168 | 298.8382 | 9.39E−02 | −6.39E−01 | 1 | Unchanged Low | |
| O15235 | L10gh1 | mitochondrial ribosomal protein S | 355.1234 | 467 | 353.2438 | 391.6738 | −7.66E−03 | 3.94E−01 | 1 | Unchanged Low | |
| Q9Y3E0 | A02ef2 | CGI-141 protein | 152.8668 | 320 | 353.6986 | 275.4907 | 1.21E+00 | 1.07E+00 | 1 | Unchanged Low | |
| Q9NQZ5 | H19gh4 | START domain containing 7 | 277.2941 | 280 | 353.7264 | 303.5369 | 3.51E−01 | 1.19E−02 | 1 | Unchanged Low | |
| Q9UHW4 | D24ef8 | protein x 0001 | 266.9848 | 291 | 353.7644 | 303.9535 | 4.06E−01 | 1.25E−01 | 1 | Unchanged Low | |
| P82914 | P04gh7 | mitochondrial ribosomal protein S | 444.1517 | 417 | 354.4965 | 405.0567 | −3.25E−01 | −9.27E−02 | 1 | Unchanged Low | |
| P25787 | L11ef7 | proteasome (prosome: macropai | 432.9379 | 526 | 355.1116 | 437.9448 | −2.86E−01 | 2.80E−01 | 1 | Unchanged Low | |
| P26447 | D02cd1 | S100 calcium binding protein A4 | 198.7401 | 273 | 355.1396 | 275.5734 | 8.38E−01 | 4.57E−01 | 1 | Unchanged Low | |
| Q9Y605 | P02gh8 | T-cell activation protein | 411.1148 | 356 | 355.1531 | 373.9359 | −2.11E−01 | −2.10E−01 | 1 | Unchanged Low | |
| Q14776 | K14cd4 | leucine-zipper-like transcriptiona | 202.0365 | 273 | 355.4522 | 276.788 | 8.15E−01 | 4.34E−01 | 1 | Unchanged Low | |
| P30837 | C02ab2 | aldehyde dehydrogenase 1 famil | 631.0407 | 451 | 355.7998 | 479.1158 | −8.27E−01 | −4.86E−01 | 1 | Unchanged Low | |
| P09668 | L03ef7 | cathepsin H | 460.8488 | 564 | 356.7735 | 460.5856 | −3.69E−01 | 2.92E−01 | 1 | Unchanged Low | |
| Q02313 | J02gh6 | Krueppel-related zinc finger prot | 402.289 | 391 | 356.9162 | 383.4797 | −1.73E−01 | −4.02E−02 | 1 | Unchanged Low | |
| Q13148 | H11cd7 | TAR DNA binding protein | 322.5816 | 353 | 357.1404 | 344.328 | 1.47E−01 | 1.31E−01 | 1 | Unchanged Low | |
| Q16645 | K24ab5 | FK506 binding protein 1B (12.6 k | 216.7704 | 486 | 357.2698 | 353.4204 | 7.21E−01 | 1.17E+00 | 1 | Unchanged Low | |
| | N02gh8 | protein phosphatase 1; regulator | 77.52993 | 71.1 | 357.2714 | 188.643 | 2.20E+00 | −1.24E−01 | 1 | Unchanged Low | |
| P57739 | F18gh1 | claudin 2 | 188.162 | 238 | 357.443 | 261.2968 | 9.26E−01 | 3.41E−01 | 1 | Unchanged Low | |
| O60568 | F13cd4 | procollagen-lysine; 2-oxoglutarat | 332.5306 | 269 | 357.5594 | 319.8117 | 1.05E−01 | −3.04E−01 | 1 | Unchanged Low | |
| Q9HAU4 | N11gh5 | E3 ubiquitin ligase SMURF2 | 425.5948 | 665 | 357.6911 | 482.7167 | −2.51E−01 | 6.44E−01 | 1 | Unchanged Low | |
| P49914 | J01cd6 | 5:10-methenyltetrahydrofolate sy | 340.2344 | 238 | 358.0826 | 312.027 | 7.38E−02 | −5.17E−01 | 1 | Unchanged Low | |
| O60510 | E11cd8 | atrophin-1 interacting protein 1; a | 171.9425 | 265 | 358.342 | 265.2168 | 1.06E+00 | 6.26E−01 | 1 | Unchanged Low | |
| Q9BX68 | L15gh8 | histidine triad nucleotide binding | 207.0703 | 360 | 358.5328 | 308.6409 | 7.92E−01 | 7.99E−01 | 1 | Unchanged Low | |
| P29084 | B02ab5 | general transcription factor IIE: p | 261.3572 | 225 | 358.821 | 281.687 | 4.57E−01 | −2.17E−01 | 1 | Unchanged Low | |
| P09526 | C05ef6 | RAP1B: member of RAS oncoge | 320.4156 | 355 | 359.0861 | 344.9493 | 1.64E−01 | 1.49E−01 | 1 | Unchanged Low | |
| O15471 | P01ef4 | leukocyte immunoglobulin-like re | 219.8277 | 260 | 359.2909 | 279.8225 | 7.09E−01 | 2.44E−01 | 1 | Unchanged Low | |
| Q9UEE9 | K03ab3 | craniofacial development protein | 124.0304 | 245 | 359.5047 | 242.664 | 1.54E+00 | 9.79E−01 | 1 | Unchanged Low | |
| Q15011 | L03ef3 | homocysteine-inducible; endopla | 352.4459 | 183 | 359.7095 | 298.5074 | 2.94E−02 | −9.43E−01 | 1 | Unchanged Low | |
| Q9BWG8 | A13gh6 | hypothetical protein MGC2491 | 170.1162 | 203 | 360.534 | 244.6389 | 1.08E+00 | 2.57E−01 | 1 | Unchanged Low | |
| Q9Y397 | A04ef2 | zinc finger; DHHC domain contai | 431.1743 | 495 | 360.6182 | 428.8727 | −2.58E−01 | 1.99E−01 | 1 | Unchanged Low | |
| Q14596 | H17ab7 | membrane component: chromos | 303.2476 | 402 | 361.5265 | 355.7499 | 2.54E−01 | 4.08E−01 | 1 | Unchanged Low | |
| P23497 | C13cd2 | nuclear antigen Sp100 | 331.8296 | 491 | 361.9825 | 394.8464 | 1.25E−01 | 5.64E−01 | 1 | Unchanged Low | |
| Q16633 | A24cd1 | POU domain: class 2: associating | 103.1231 | 99.3 | 362.0007 | 188.1549 | 1.81E+00 | −5.39E−02 | 1 | Unchanged Low | |
| Q99961 | J12cd1 | SH3-domain GRB2-like 1 | 231.066 | 247 | 362.8536 | 280.235 | 6.51E−01 | 9.47E−02 | 1 | Unchanged Low | |
| Q07021 | N20ab3 | complement component 1: q sub | 471.8742 | 202 | 363.3385 | 345.9018 | −3.77E−01 | −1.22E+00 | 1 | Unchanged Low | |
| Q16513 | E04cd1 | protein kinase C-like 2 | 546.0111 | 561 | 364.4852 | 490.5792 | −5.83E−01 | 3.97E−02 | 1 | Unchanged Low | |
| Q9H070 | H10cd8 | adaptor-related protein complex: | 353.2901 | 278 | 364.7541 | 331.9695 | 4.61E−02 | −3.46E−01 | 1 | Unchanged Low | |
| Q13227 | A16ab5 | G protein pathway suppressor 2 | 557.5311 | 352 | 364.8449 | 424.6909 | −6.12E−01 | −6.65E−01 | 1 | Unchanged Low | |
| P20339 | M03ef5 | RAB5A; member RAS oncogene | 404.6579 | 664 | 365.7139 | 478.1731 | −1.46E−01 | 7.15E−01 | 1 | Unchanged Low | |
| Q9NXX4 | G15gh2 | hypothetical protein FLJ20005 | 155.2731 | 246 | 365.9622 | 255.5867 | 1.24E+00 | 6.61E−01 | 1 | Unchanged Low | |
| Q92900 | N17cd1 | regulator of nonsense transcripts | 328.2926 | 316 | 366.2931 | 336.7297 | 1.58E−01 | −5.69E−02 | 1 | Unchanged Low | |
| Q9H2K4 | I05gh3 | chromosome 11 open reading fra | 198.6548 | 155 | 366.4866 | 240.051 | 8.83E−01 | −3.58E−01 | 1 | Unchanged Low | |
| O14917 | E17ef4 | protocadherin 17 | 250.3859 | 311 | 366.6879 | 309.398 | 5.50E−01 | 3.13E−01 | 1 | Unchanged Low | |
| Q9NZ88 | J06gh3 | chromosome 6 open reading fra | 199.6701 | 362 | 367.2681 | 309.7781 | 8.79E−01 | 8.60E−01 | 1 | Unchanged Low | |
| Q9BYC8 | C15gh8 | mitochondrial ribosomal protein | 257.9414 | 429 | 367.9727 | 351.4981 | 5.13E−01 | 7.33E−01 | 1 | Unchanged Low | |
| Q15311 | C21cd7 | ralA binding protein 1 | 295.4648 | 411 | 368.6028 | 358.3107 | 3.19E−01 | 4.76E−01 | 1 | Unchanged Low | |
| Q9NWE9 | E02gh3 | hypothetical protein FLJ10074 | 455.3747 | 383 | 369.4489 | 402.6031 | −3.02E−01 | −2.50E−01 | 1 | Unchanged Low | |
| O95571 | G22ef3 | protein expressed in thyroid | 398.0124 | 507 | 369.925 | 425.0962 | −1.06E−01 | 3.50E−01 | 1 | Unchanged Low | |
| P36941 | A09ab7 | lymphotoxin beta receptor (TNFF | 403.0565 | 454 | 370.0343 | 409.094 | −1.23E−01 | 1.72E−01 | 1 | Unchanged Low | |
| P49790 | F11cd5 | nucleoporin 153kD | 264.6175 | 316 | 370.2793 | 317.116 | 4.85E−01 | 2.58E−01 | 1 | Unchanged Low | |
| Q99607 | C06ab4 | E74-like factor 4 (ets domain tra | 267.135 | 268 | 371.3515 | 302.1703 | 4.75E−01 | 4.79E−03 | 1 | Unchanged Low | |
| Q9UIV1 | H05cd6 | CCR4-NOT transcription comple: | 201.0959 | 465 | 371.6771 | 345.9619 | 8.86E−01 | 1.21E+00 | 1 | Unchanged Low | |
| Q9Y3E4 | O13ef2 | CGI-146 protein | 107.5317 | 262 | 371.8333 | 247.1535 | 1.79E+00 | 1.29E+00 | 1 | Unchanged Low | |
| P19367 | D17ab3 | hexokinase 1 | 512.0647 | 215 | 371.9817 | 366.4437 | −4.61E−01 | −1.25E+00 | 1 | Unchanged Low | |
| Q14250 | J15cd5 | enigma (LIM domain protein) | 229.1944 | 218 | 371.9927 | 272.9152 | 6.99E−01 | −7.52E−02 | 1 | Unchanged Low | |
| Q9UL33 | B20ef2 | unknown | 343.1533 | 636 | 372.4427 | 450.4535 | 1.18E−01 | 8.90E−01 | 1 | Unchanged Low | |
| Q15019 | H03ef5 | neural precursor cell expressed: | 489.0601 | 274 | 372.9279 | 378.666 | −3.91E−01 | −8.36E−01 | 1 Unchan9ed Low | | |
| 013084 | H05cd8 | melanoma-associated antigen re | 421.8259 | 278 | 373.0512 | 357.6504 | −1.77E−01 | −6.01E−01 | 1 | Unchanged Low | |
| Q9UMR2 | O18cd7 | DEAD/H (Asp-Glu-Ala-Asp/His) | 383.654 | 423 | 373.0622 | 393.1434 | −4.04E−02 | 1.40E−01 | 1 | Unchanged Low | |
| P55855 | H06cd2 | SMT3 suppressor of mif two 3 ho | 454.9011 | 444 | 373.596 | 424.0867 | −2.84E−01 | −3.58E−02 | 1 | Unchanged Low | |
| Q13155 | G20cd4 | JTV1 gene | 322.6641 | 409 | 373.7416 | 368.4293 | 2.12E−01 | 3.42E−01 | 1 | Unchanged Low | |
| P78371 | H11cd6 | chaperonin containing TCP1: sub | 480.5464 | 430 | 374.014 | 428.2475 | −3.62E−01 | −1.60E−01 | 1 | Unchanged Low | |
| P08173 | P17ab3 | cholinergic receptor; muscarinic | 444.2461 | 514 | 374.4117 | 444.3189 | −2.47E−01 | 2.11E−01 | 1 | Unchanged Low | |
| O60478 | E02cd2 | transmembrane 7 superfamily me | 274.7639 | 438 | 374.61 | 362.4989 | 4.47E−01 | 6.73E−01 | 1 | Unchanged Low | |
| O60429 | D16gh4 | hypothetical protein FLJ12886 | 142.8322 | 257 | 375.0573 | 258.282 | 1.39E+00 | 8.47E−01 | 1 | Unchanged Low | |
| O75935 | O04cd7 | dynactin 3 (p22) | 408.9696 | 432 | 375.1224 | 405.4281 | −1.25E−01 | 7.97E−02 | 1 | Unchanged Low | |
| Q13147 | J05ab2 | abl-interactor 2 | 299.098 | 249 | 375.379 | 307.9098 | 3.28E−01 | −2.63E−01 | 1 | Unchanged Low | |
| Q9NPH4 | E14gh3 | LUC7-like (S. cerevisiae) | 207.38 | 302 | 376.3768 | 295.4166 | 8.60E−01 | 5.45E−01 | 1 | Unchanged Low | |
| Q9UJ96 | C13cd8 | potassium voltage-gated channe | 582.717 | 448 | 376.4373 | 468.9343 | −6.30E−01 | −3.80E−01 | 1 | Unchanged Low | |
| P17081 | C13ef6 | likely ortholog of mouse TC10-al | 293.1604 | 417 | 376.4996 | 362.2816 | 3.61E−01 | 5.09E−01 | 1 | Unchanged Low | |
| P11166 | J05ef5 | solute carrier family 2 (facilitated | 351.7333 | 393 | 376.564 | 373.8343 | 9.84E−02 | 1.61E−01 | 1 | Unchanged Low | |
| Q14820 | D12ef6 | splicing factor 1 | 382.846 | 408 | 376.7779 | 389.2167 | −2.30E−02 | 9.19E−02 | 1 | Unchanged Low | |
| Q92935 | I06ab5 | exostoses (multiple)-like 1 | 659.9005 | 276 | 376.8548 | 437.6869 | −8.08E−01 | −1.26E+00 | 1 | Unchanged Low | |
| P31749 | E20ef6 | v-akl murine thymoma viral onco | 385.4669 | 303 | 376.9169 | 355.1851 | −3.24E−02 | −3.46E−01 | 1 | Unchanged Low | |
| Q13454 | L05cd7 | Putative prostate cancer tumor s | 326.9146 | 363 | 377.0852 | 355.5691 | 2.06E−01 | 1.50E−01 | 1 | Unchanged Low | |
| P35520 | C24ab3 | cystathionine-beta-synthase | 70.85109 | 68.1 | 377.1148 | 172.0203 | 2.41E+00 | −5.72E−02 | 1 | Unchanged Low | |
| P53602 | M01ab7 | mevalonate (diphospho) decarbo | 682.0309 | 178 | 378.114 | 412.6357 | −8.51E−01 | −1.94E+00 | 1 | Unchanged Low | |
| Q9H7G3 | C04gh6 | mitochondrial ribosomal protein | 333.6677 | 207 | 378.1396 | 306.3867 | 1.81E−01 | −6.86E−01 | 1 | Unchanged Low | |
| Q13123 | C06ab7 | IK cytokine; down-regulator of H | 257.7332 | 439 | 378.1806 | 358.3456 | 5.53E−01 | 7.69E−01 | 1 | Unchanged Low | |
| O14744 | G14cd6 | SKB1 homolog (S. pombe) | 348.0026 | 410 | 378.4165 | 378.8177 | 1.21E−01 | 2.37E−01 | 1 | Unchanged Low | |
| Q9Y566 | D16ef8 | SH3 and multiple ankyrin repeat | 209.2687 | 281 | 379.5197 | 290.0715 | 8.59E−01 | 4.27E−01 | 1 | Unchanged Low | |
| Q9BUJ9 | A03gh7 | Mov10: Moloney leukemia virus | 385.0517 | 472 | 380.308 | 412.4352 | −1.79E−02 | 2.64E−01 | 1 | Unchanged Low | |
| P51790 | I02ab5 | chloride channel 3 | 330.2251 | 237 | 380.3105 | 315.7621 | 2.04E−01 | −4.80E−01 | 1 | Unchanged Low | |
| O14547 | J09cd6 | PRP8 pre-mRNA processing fact | 239.0005 | 256 | 380.5886 | 291.9777 | 6.71E−01 | 1.01E−01 | 1 | Unchanged Low | |
| Q99753 | A05cd7 | cisplatin resistance associated | 257.1873 | 397 | 380.7894 | 345.0384 | 5.66E−01 | 6.27E−01 | 1 | Unchanged Low | |
| Q9NQC8 | H15gh4 | hypothetical protein LOC56912 | 185.2607 | 276 | 381.0416 | 260.7773 | 1.04E+00 | 5.75E−01 | 1 | Unchanged Low | |
| O43919 | M01ef6 | linker for activation of T cells | 206.6586 | 105 | 381.2059 | 230.823 | 8.83E−01 | −9.82E−01 | 1 | Unchanged Low | |
| P78345 | F15cd6 | ribonuclease P (38kD) | 321.791 | 530 | 381.5687 | 411.2575 | 2.46E−01 | 7.21E−01 | 1 | Unchanged Low | |
| P54257 | A03ef5 | huntingtin-associaled protein 1 ( | 450.2874 | 322 | 382.6029 | 384.9948 | −2.35E−01 | −4.83E−01 | 1 | Unchanged Low | |
| Q9Y3F4 | G18cd7 | unr-interacting protein | 390.9437 | 335 | 382.6142 | 369.3734 | −3.11E−02 | −2.25E−01 | 1 | Unchanged Low | |
| P29536 | F14cd7 | leiomodin 1 (smooth muscle) | 343.3731 | 209 | 382.9438 | 311.936 | 1.57E−01 | −7.13E−01 | 1 | Unchanged Low | |
| O75925 | K03cd4 | protein inhibitor of activated STA | 423.9992 | 606 | 383.7943 | 471.2944 | −1.44E−01 | 5.15E−01 | 1 | Unchanged Low | |
| Q9UMX5 | J23ef4 | secreted protein of unknown fun | 344.8668 | 306 | 384.5676 | 345.2233 | 1.57E−01 | −1.71E−01 | 1 | Unchanged Low | |
| Q9UMZ9 | N02cd7 | interleukin enhancer binding fact | 348.7866 | 278 | 384.7729 | 337.1554 | 1.42E−01 | −3.28E−01 | 1 | Unchanged Low | |
| Q92905 | P03ef6 | COP9 constitutive photomorphog | 257.5597 | 392 | 386.1452 | 345.1965 | 5.84E−01 | 6.06E−01 | 1 | Unchanged Low | |
| Q9NSD7 | L20ef1 | G-protein coupled receptor SAL | 734.6282 | 277 | 386.7193 | 466.2798 | −9.26E−01 | −1.40E+00 | 1 | Unchanged Low | |
| Q9P2W3 | G01ef3 | guanine nucleotide binding prote | 70.71144 | 81.4 | 386.7442 | 179.6321 | 2.45E+00 | 2.04E−01 | 1 | Unchanged Low | |
| Q9BVI7 | N14gh5 | mitochondrial ribosomal protein S | 367.7177 | 520 | 386.9198 | 424.8293 | 7.34E−02 | 4.99E−01 | 1 | Unchanged Low | |
| Q9Y4L1 | B23cd6 | oxygen regulated protein (150kD | 413.0575 | 372 | 387.5372 | 390.9975 | −9.20E−02 | −1.49E−01 | 1 | Unchanged Low | |
| Q02363 | F18ab7 | inhibitor of DNA binding 2; domi | 484.7132 | 359 | 388.2552 | 410.6421 | −3.20E−01 | −4.33E−01 | 1 | Unchanged Low | |
| Q9Y5J8 | E06cd8 | transtocase of inner mitochondria | 440.2763 | 542 | 388.4554 | 456.8557 | −1.81E−01 | 2.99E−01 | 1 | Unchanged Low | |
| | E22ef1 | G protein-coupled receptor 26 | 499.1316 | 450 | 389.582 | 446.2248 | −3.57E−01 | −1.50E−01 | 1 | Unchanged Low | |
| Q99501 | N11cd6 | growth arrest-specific 2 like 1 | 359.8065 | 540 | 389.6398 | 429.6515 | 1.15E−01 | 5.84E−01 | 1 | Unchanged Low | |
| Q00613 | M17ef5 | heat shock transcription factor 1 | 363.3847 | 375 | 389.8895 | 376.2292 | 1.01E−01 | 4.71E−02 | 1 | Unchanged Low | |
| Q9H5Z1 | H16gh4 | DEAD/H (Asp-Glu-Ala-Asp/His) | 136.5899 | 124 | 389.9835 | 217.0007 | 1.51E+00 | −1.35E−01 | 1 | Unchanged Low | |
| P09661 | P24cd1 | small nuclear ribonucleoprotein | 441.1409 | 115 | 390.2085 | 315.5701 | −1.77E−01 | −1.94E+00 | 1 | Unchanged Low | |
| P54709 | P05ef5 | ATPase; Na+/K+ transporting; be | 425.6647 | 432 | 391.8135 | 416.5779 | −1.20E−01 | 2.22E−02 | 1 | Unchanged Low | |
| P13164 | M06ef5 | interferon induced transmembran | 45.13817 | 256 | 392.1204 | 231.1336 | 3.12E+00 | 2.50E+00 | 1 | Unchanged Low | |
| Q9Y6E2 | A23ef8 | HSPC028 protein | 329.8857 | 363 | 392.2431 | 361.6248 | 2.50E−01 | 1.37E−01 | 1 | Unchanged Low | |
| Q9NVG2 | B13gh3 | vacuolar protein sorting 35 (yeas | 359.0233 | 557 | 392.2574 | 436.0959 | 1.28E−01 | 6.34E−01 | 1 | Unchanged Low | |
| P27448 | E10ef5 | MAP/microtubule affinity-regulati | 397.7845 | 369 | 392.3918 | 386.4633 | −1.97E−02 | −1.08E−01 | 1 | Unchanged Low | |
| O15438 | P18cd3 | ATP-binding cassette: sub-family | 618.0409 | 488 | 392.8159 | 499.6643 | −6.54E−01 | −3.40E−01 | 1 | Unchanged Low | |
| Q9UL48 | I04ef2 | CK2 interacting protein 1; HQ002 | 176.5553 | 215 | 393.4111 | 261.8209 | 1.16E+00 | 2.88E−01 | 1 | Unchanged Low | |
| O94763 | A11cd4 | RPB5-mediating protein | 333.6215 | 533 | 393.9448 | 420.3384 | 2.40E−01 | 6.77E−01 | 1 | Unchanged Low | |
| Q13190 | I19cd2 | syntaxin 5A | 179.6148 | 235 | 393.985 | 269.5995 | 1.13E+00 | 3.89E−01 | 1 | Unchanged Low | |
| Q13122 | D21ef3 | EBNA-2 co-activator (100kD) | 381.3069 | 375 | 394.5999 | 383.7948 | 4.94E−02 | −2.22E−02 | 1 | Unchanged Low | |
| O75312 | L01cd4 | zinc finger protein 259 | 348.7542 | 422 | 394.8016 | 388.3905 | 1.79E−01 | 2.74E−01 | 1 | Unchanged Low | |
| P37837 | D20cd2 | transaldolase 1 | 399.2717 | 326 | 395.693 | 373.7275 | −1.30E−02 | −2.92E−01 | 1 | Unchanged Low | |
| O75762 | G19cd6 | ankyrin-like with tranamembrane | 222.8517 | 364 | 395.7427 | 327.3962 | 8.28E−01 | 7.06E−01 | 1 | Unchanged Low | |
| Q99969 | K19ef3 | retinoic acid receptor responder | 283.5705 | 257 | 396.0287 | 312.0672 | 4.82E−01 | −1.44E−01 | 1 | Unchanged Low | |
| Q9UNP2 | H15cd2 | solute carrier family 1 (neutral an | 149.1019 | 138 | 396.5014 | 227.7665 | 1.41E+00 | −1.15E−01 | 1 | Unchanged Low | |
| P56524 | C23cd6 | histone deacetylase 4 | 328.6037 | 303 | 397.1317 | 342.9334 | 2.73E−01 | −1.17E−01 | 1 | Unchanged Low | |
| Q9H644 | C19gh6 | hypothetical protein MGC5585 | 140.9811 | 228 | 397.2624 | 255.3931 | 1.49E+00 | 6.93E−01 | 1 | Unchanged Low | |
| Q13627 | O19ab4 | dual-specificity tyrosine-(Y)-phos | 498.484 | 327 | 397.3922 | 407.7774 | −3.27E−01 | −6.06E−01 | 1 | Unchanged Low | |
| Q03405 | O01ef5 | plasminogen activator; urokinase | 500.1391 | 396 | 398.2456 | 431.3524 | −3.29E−01 | −3.38E−01 | 1 | Unchanged Low | |
| Q9NPQ8 | L18gh4 | likely ortholog of mouse synembr | 372.8251 | 443 | 398.4083 | 404.6217 | 9.57E−02 | 2.48E−01 | 1 | Unchanged Low | |
| Q15375 | A10ab5 | EphA7 | 243.0574 | 290 | 398.5414 | 310.569 | 7.13E−01 | 2.55E−01 | 1 | Unchanged Low | |
| P05114 | G04ab7 | high-mobility group (nonhistone | 196.8334 | 278 | 398.7327 | 291.2748 | 1.02E+00 | 4.99E−01 | 1 | Unchanged Low | |
| Q9UKU4 | C13ef2 | retinal shod-chain dehydrogenas | 370.8225 | 497 | 399.4413 | 422.4244 | 1.07E−01 | 4.23E−01 | 1 | Unchanged Low | |
| P08195 | E21ab7 | solute carrier family 3 (activators | 442.5453 | 345 | 399.5678 | 395.768 | −1.47E−01 | −3.58E−01 | 1 | Unchanged Low | |
| Q15545 | J11cd2 | TAF7 RNA polymerase II; TATA | 400.689 | 526 | 401.1308 | 442.5426 | 1.59E−03 | 3.92E−01 | 1 | Unchanged Low | |
| | D24gh8 | hypothetical protein FLJ14511 | 246.3293 | 225 | 401.3816 | 290.8781 | 7.04E−01 | −1.31E−01 | 1 | Unchanged Low | |
| Q05397 | L20ef5 | PTK2 protein tyrosine kinase 2 | 389.8718 | 440 | 401.4713 | 410.2909 | 4.23E−02 | 1.73E−01 | 1 | Unchanged Low | |
| Q9Y6G1 | C21ef8 | PTD011 protein | 434.6906 | 483 | 401.5119 | 439.8467 | −1.15E−01 | 1.53E−01 | 1 | Unchanged Low | |
| P41212 | O23ef5 | ets variant gene 6 (TEL oncogen | 315.7331 | 502 | 401.8428 | 406.6035 | 3.48E−01 | 6.70E−01 | 1 | Unchanged Low | |
| O14737 | G05cd5 | programmed cell death 5 | 498.729 | 430 | 402.3091 | 443.7457 | −3.10E−01 | −2.13E−01 | 1 | Unchanged Low | |
| Q9UHB9 | A10ef3 | signal recognition particle 68kD | 212.5357 | 247 | 402.3276 | 287.2236 | 9.21E−01 | 2.16E−01 | 1 | Unchanged Low | |
| Q96D21 | C20ef8 | RASD family: member 2 | 395.909 | 486 | 402.4304 | 427.9499 | 2.36E−02 | 2.94E−01 | 1 | Unchanged Low | |
| Q9Y4I1 | J05ef1 | myosin VA (heavy polypeptide 12 | 371.5954 | 353 | 402.453 | 375.8247 | 1.15E−01 | −7.23E−02 | 1 | Unchanged Low | |
| Q9Y5L4 | L14ef7 | translocase of inner mitochondria | 291.7019 | 369 | 404.0738 | 354.9111 | 4.70E−01 | 3.39E−01 | 1 | Unchanged Low | |
| O75439 | N22cd4 | peptidase (mitochondrial process | 512.9004 | 184 | 404.5715 | 367.0789 | −3.42E−01 | −1.48E+00 | 1 | Unchanged Low | |
| P55010 | K22ab4 | eukaryotic translation initiation fa | 427.4989 | 296 | 405.4996 | 376.3076 | −7.62E−02 | −5.31E−01 | 1 | Unchanged Low | |
| Q9BY44 | G10gh8 | CDA02 protein | 453.6064 | 401 | 405.956 | 420.1404 | −1.60E−01 | −1.78E−01 | 1 | Unchanged Low | |
| Q13164 | B21ef5 | mitogen-activated protein kinase | 209.1026 | 256 | 406.234 | 290.5304 | 9.58E−01 | 2.93E−01 | 1 | Unchanged Low | |
| P53667 | P08ab6 | LIM domain kinase 1 | 359.4655 | 392 | 406.5183 | 386.0829 | 1.77E−01 | 1.26E−01 | 1 | Unchanged Low | |
| Q9UNH6 | F05ef2 | sorting nexin 7 | 479.6898 | 396 | 406.7812 | 427.3762 | −2.38E−01 | −2.78E−01 | 1 | Unchanged Low | |
| O00258 | K17cd4 | tryptophan rich basic protein | 339.3172 | 500 | 407.1504 | 415.5431 | 2.63E−01 | 5.60E−01 | 1 | Unchanged Low | |
| Q9Y3I0 | I12ef3 | hypothetical protein | 458.2693 | 546 | 407.2408 | 470.3717 | −1.70E−01 | 2.52E−01 | 1 | Unchanged Low | |
| Q9Y368 | D13cd8 | parvin; beta | 114.232 | 159 | 407.3882 | 226.9908 | 1.83E+00 | 4.80E−01 | 1 | Unchanged Low | |
| P14866 | H13ab6 | heterogeneous nuclear ribonucle | 442.5489 | 274 | 407.4025 | 374.6383 | −1.19E−01 | −6.92E−01 | 1 | Unchanged Low | |
| Q13885 | K01cd3 | tubulin: beta polypeptide | 441.2669 | 413 | 407.8683 | 420.8456 | −1.14E−01 | −9.41E−02 | 1 | Unchanged Low | |
| O43765 | J06cd1 | small glutamine-rich tetratricopep | 378.9705 | 438 | 408.6453 | 408.6939 | 1.09E−01 | 2.10E−01 | 1 | Unchanged Low | |
| O14632 | B12ef4 | homeodomain interacting protein | 394.449 | 535 | 408.8773 | 446.1081 | 5.18E−02 | 4.40E−01 | 1 | Unchanged Low | |
| P31939 | L10ab2 | 5-aminoimidazole-4-carboxamide | 501.3028 | 386 | 409.3069 | 432.286 | −2.92E−01 | −3.76E−01 | 1 | Unchanged Low | |
| P28331 | P18ab8 | NADH dehydrogenase (ubiquino | 490.4347 | 417 | 409.3283 | 438.7901 | −2.61E−01 | −2.35E−01 | 1 | Unchanged Low | |
| P41970 | P05ef6 | ELK3; ETS-domain protein (SRF | 205.9453 | 359 | 410.8037 | 325.3706 | 9.96E−01 | 8.03E−01 | 1 | Unchanged Low | |
| Q92973 | J22ab6 | karyopherin (importin) beta 2 | 393.6551 | 234 | 410.9425 | 346.1733 | 6.20E−02 | −7.51E−01 | 1 | Unchanged Low | |
| P36507 | J10ef5 | mitogen-activated protein kinase | 395.22 | 437 | 411.4052 | 414.6953 | 5.79E−02 | 1.46E−01 | 1 | Unchanged Low | |
| Q16512 | F21ab8 | protein kinase C-like 1 | 456.2727 | 486 | 411.8245 | 451.4528 | −1.48E−01 | 9.18E−02 | 1 | Unchanged Low | |
| O43447 | M08cd6 | peptidyl prolyl isomerase H (cycl | 179.441 | 222 | 411.9165 | 271.0162 | 1.20E+00 | 3.05E−01 | 1 | Unchanged Low | |
| P30040 | M13ef3 | chromosome 12 open reading fra | 346.6792 | 296 | 412.2064 | 351.5665 | 2.50E−01 | −2.29E−01 | 1 | Unchanged Low | |
| O95399 | C19cd7 | urotensin 2 | 452.0721 | 366 | 413.1451 | 410.4914 | −1.30E−01 | −3.04E−01 | 1 | Unchanged Low | |
| | M01ab3 | UDP-Gal:betaGal beta 1;3-galac | 499.9529 | 290 | 413.3454 | 401.0113 | −2.74E−01 | −7.87E−01 | 1 | Unchanged Low | |
| Q12792 | N11ab8 | protein tyrosine kinase 9 | 476.3952 | 364 | 413.4208 | 418.007 | −2.05E−01 | −3.87E−01 | 1 | Unchanged Low | |
| Q14318 | J02cd7 | FK506 binding protein 8 (38kD) | 461.2322 | 432 | 413.4238 | 435.6453 | −1.58E−01 | −9.35E−02 | 1 | Unchanged Low | |
| O75955 | H10cd5 | flotillin 1 | 304.6674 | 310 | 415.4364 | 343.496 | 4.47E−01 | 2.68E−02 | 1 | Unchanged Low | |
| Q9BVY8 | B09gh7 | hypothetical protein MGC5499 | 490.2382 | 417 | 416.02 | 440.9559 | −2.37E−01 | −2.35E−01 | 1 | Unchanged Low | |
| Q16288 | O19ef5 | neurotrophic tyrosine kinase; rec | 126.7467 | 186 | 416.4128 | 243.0358 | 1.72E+00 | 5.53E−01 | 1 | Unchanged Low | |
| O75380 | J20ab8 | NADH dehydrogenase (ubiquino | 449.5778 | 413 | 416.4836 | 426.4999 | −1.10E−01 | −1.21E−01 | 1 | Unchanged Low | |
| Q9BY51 | B15gh8 | hypothetical protein GL009 | 429.1998 | 354 | 417.3575 | 400.0464 | −4.04E−02 | −2.80E−01 | 1 | Unchanged Low | |
| Q9BVK3 | E19gh6 | hypothetical protein MGC2463 | 462.5828 | 387 | 417.7046 | 422.5145 | −1.47E−01 | −2.56E−01 | 1 | Unchanged Low | |
| Q9P0N3 | A24ef2 | hypothetical protein FLJ10597 | 137.7487 | 289 | 417.999 | 281.424 | 1.60E+00 | 1.07E+00 | 1 | Unchanged Low | |
| P24534 | D03ab4 | eukaryotic translation elongation | 347.2405 | 328 | 418.0152 | 364.5026 | 2.68E−01 | −8.11E−02 | 1 | Unchanged Low | |
| P49336 | C01ab4 | cyclin-dependent kinase 8 | 426.3751 | 281 | 418.121 | 375.2575 | −2.62E−02 | −6.00E−01 | 1 | Unchanged Low | |
| P34130 | P16cd2 | neurotrophin 5 (neurotrophin 4/5 | 327.4146 | 223 | 418.6011 | 323.0643 | 3.54E−01 | −5.53E−01 | 1 | Unchanged Low | |
| O75425 | P06gh5 | hypothetical protein AF053356_{—} | 340.4357 | 339 | 419.3637 | 366.1692 | 3.01E−01 | −7.34E−03 | 1 | Unchanged Low | |
| Q15293 | F16ab8 | reticulocalbin 1; EF-hand calcium | 241.6383 | 250 | 420.5597 | 304.1243 | 7.99E−01 | 5.01E−02 | 1 | Unchanged Low | |
| O95376 | D08ab2 | ariadne homolog 2 (Drosophila) | 254.1122 | 322 | 420.8229 | 332.2465 | 7.28E−01 | 3.41E−01 | 1 | Unchanged Low | |
| O75489 | J16ab8 | NADH dehydrogenase (ubiquino | 375.7034 | 327 | 422.3772 | 375.1108 | 1.69E−01 | −1.99E−01 | 1 | Unchanged Low | |
| P28482 | B15eF5 | mitogen-activated protein kinase | 493.835 | 452 | 422.4625 | 456.1064 | −2.25E−01 | −1.28E−01 | 1 | Unchanged Low | |
| P20823 | B09cd1 | transcription factor 1; hepatic; LF | 322.4782 | 527 | 423.4405 | 424.3654 | 3.93E−01 | 7.09E−01 | 1 | Unchanged Low | |
| O15250 | I04cd4 | X-prolyl aminopeptidase (aminop | 524.8188 | 542 | 423.7387 | 496.7532 | −3.09E−01 | 4.57E−02 | 1 | Unchanged Low | |
| P55769 | P22ab8 | NHP2 non-histone chromosome | 137.3557 | 479 | 424.0031 | 346.681 | 1.63E+00 | 1.80E+00 | 1 | Unchanged Low | |
| O94817 | N21ab2 | APG12 autophagy 12-like (S. cer | 631.6055 | 402 | 424.3577 | 485.9574 | −5.74E−01 | −6.52E−01 | 1 | Unchanged Low | |
| O75352 | K08cd5 | mannose-P-dolichol utilization de | 272.444 | 523 | 424.8266 | 406.7998 | 6.41E−01 | 9.41E−01 | 1 | Unchanged Low | |
| Q16585 | I16cd1 | sarcoglycan: beta (43kD dystropl | 256.2113 | 293 | 425.1172 | 324.9166 | 7.31E−01 | 1.96E−01 | 1 | Unchanged Low | |
| Q9H7X7 | B16gh5 | hypothetical protein FLJ14117 | 261.9578 | 463 | 425.3725 | 383.5805 | 6.99E−01 | 8.23E−01 | 1 | Unchanged Low | |
| Q15173 | I16cd6 | progesterone receptor membran | 295.0481 | 244 | 425.8728 | 321.6602 | 5.29E−01 | −2.74E−01 | 1 | Unchanged Low | |
| Q9GZX9 | A16gh4 | twisted gastrulation | 84.61732 | 147 | 426.1124 | 219.3062 | 2.33E+00 | 7.99E−01 | 1 | Unchanged Low | |
| Q16594 | M09cd2 | TAF9 RNA polymerase II; TATA | 171.3507 | 520 | 426.7606 | 372.6331 | 1.32E+00 | 1.60E+00 | 1 | Unchanged Low | |
| O00479 | O17gh1 | high-mobility group (nonhistone | 265.695 | 354 | 426.7642 | 348.9168 | 6.84E−01 | 4.15E−01 | 1 | Unchanged Low | |
| P54760 | C22ab5 | EphB4 | 528.7066 | 413 | 427.0543 | 456.4179 | −3.08E−01 | −3.55E−01 | 1 | Unchanged Low | |
| P25490 | F06ef6 | YY1 transcription factor | 481.2844 | 530 | 427.184 | 479.4548 | −1.72E−01 | 1.39E−01 | 1 | Unchanged Low | |
| Q9UJY0 | B07ef3 | putative nucleotide binding prote | 505.9493 | 431 | 427.3544 | 454.8441 | −2.44E−01 | −2.31E−01 | 1 | Unchanged Low | |
| Q09666 | A22ef1 | AHNAK nucleoprotein (desmoyol | 392.0671 | 399 | 427.3828 | 406.041 | 1.24E−01 | 2.41E−02 | 1 | Unchanged Low | |
| O15305 | P22ab7 | phosphomannomutase 2 | 325.0777 | 480 | 428.3778 | 411.2528 | 3.98E−01 | 5.63E−01 | 1 | Unchanged Low | |
| O60492 | M09ab4 | 7-dehydrocholesterol reductase | 358.5228 | 213 | 428.5923 | 343.3316 | 1.42E−01 | −8.68E−01 | 1 | Unchanged Low | |
| Q9UND3 | G11cd6 | nuclear pore complex interacting | 280.8175 | 312 | 428.5952 | 340.6147 | 6.10E−01 | 1.54E−01 | 1 | Unchanged Low | |
| Q9HB15 | M15gh5 | potassium channel; subfamily K: | 473.5589 | 408 | 428.9306 | 436.9131 | −1.43E−01 | −2.14E−01 | 1 | Unchanged Low | |
| Q9Y3B4 | H23ef2 | CGI-110 protein | 290.9669 | 331 | 429.1497 | 350.4808 | 5.61E−01 | 1.87E−01 | 1 | Unchanged Low | |
| Q14107 | F05ef5 | D123 gene product | 387.0465 | 405 | 429.9331 | 407.3091 | 1.52E−01 | 6.52E−02 | 1 | Unchanged Low | |
| P52435 | A22cd1 | polymerase (RNA) II (DNA direct | 527.0446 | 461 | 429.9488 | 472.7272 | −2.94E−01 | −1.93E−01 | 1 | Unchanged Low | |
| Q9Y262 | L11ef2 | eukaryotic translation initiation fa | 591.5767 | 311 | 430.6788 | 444.385 | −4.58E−01 | −9.28E−01 | 1 | Unchanged Low | |
| P10163 | L20cd2 | proline-rich protein BstNI subfam | 521.4154 | 251 | 431.1299 | 401.2036 | −2.74E−01 | −1.05E+00 | 1 | Unchanged Low | |
| P38378 | F01cd8 | protein transport protein SEC61 | 635.5838 | 369 | 431.9958 | 478.7225 | −5.57E−01 | −7.86E−01 | 1 | Unchanged Low | |
| P48735 | B06ab6 | isocitrate dehydrogenase 2 (NAD | 600.2178 | 338 | 432.1616 | 456.8577 | 4.74E−01 | −8.28E−01 | 1 | Unchanged Low | |
| Q9NPF5 | F02gh4 | DNA methyltransferase 1-associa | 451.5343 | 382 | 432.3473 | 421.964 | −6.26E−02 | −2.41E−01 | 1 | Unchanged Low | |
| O14616 | K22cd4 | zinc finger protein-like 1 | 194.5871 | 348 | 432.4885 | 324.9789 | 1.15E+00 | 8.38E−01 | 1 | Unchanged Low | |
| Q00839 | A12ab7 | heterogeneous nuclear ribonucle | 416.5145 | 410 | 432.5188 | 419.7665 | 5.44E−02 | −2.18E−02 | 1 | Unchanged Low | |
| O95563 | J15ef8 | DKFZP564B167 protein | 390.848 | 389 | 432.8082 | 404.2972 | 1.47E−01 | −5.96E−03 | 1 | Unchanged Low | |
| Q92974 | I03cd6 | rho/rac guanine nucleotide excha | 142.3392 | 124 | 433.2386 | 233.2947 | 1.61E+00 | −1.95E−01 | 1 | Unchanged Low | |
| Q9UEE5 | M07cd5 | serine/threonine kinase 17a (apo | 397.8599 | 472 | 433.4221 | 434.4129 | 1.24E−01 | 2.46E−01 | 1 | Unchanged Low | |
| Q9NX81 | O19gh3 | BTB (POZ) domain containing 2 | 175.3887 | 206 | 433.4811 | 271.6819 | 1.31E+00 | 2.33E−01 | 1 | Unchanged Low | |
| Q9NXF9 | O01gh3 | O-linked mannose beta1;2-N-ace | 477.6375 | 353 | 434.2927 | 421.54 | −1.37E−01 | −4.38E−01 | 1 | Unchanged Low | |
| P98171 | F13ef1 | Rho GTPase activating protein 4 | 368.9887 | 231 | 435.3585 | 345.0241 | 2.39E−01 | −6.77E−01 | 1 | Unchanged Low | |
| P56192 | I14ab7 | methionine-tRNA synthetase | 263.0076 | 295 | 435.894 | 331.1505 | 7.29E−01 | 1.63E−01 | 1 | Unchanged Low | |
| O95411 | K09cd5 | TGFB1-induced anti-apoptotic fa | 233.3926 | 310 | 436.2684 | 326.6136 | 9.02E−01 | 4.10E−01 | 1 | Unchanged Low | |
| P09132 | E23cd2 | signal recognition particle 19kD | 227.8193 | 595 | 436.5994 | 419.7603 | 9.38E−01 | 1.38E+00 | 1 | Unchanged Low | |
| Q92537 | O24gh1 | KIAA0247 gene product | 291.1054 | 232 | 437.1141 | 319.914 | 5.86E−01 | −3.30E−01 | 1 | Unchanged Low | |
| P49761 | G03ab4 | CDC-like kinase 3 | 458.9286 | 562 | 437.5601 | 486.0405 | −6.88E−02 | 2.91E−01 | 1 | Unchanged Low | |
| Q9HDC4 | O19gh4 | junctophilin 3 | 346.7829 | 388 | 439.282 | 391.3835 | 3.41E−01 | 1.62E−01 | 1 | Unchanged Low | |
| P98160 | A13ef7 | heparan sulfate proteoglycan 2 ( | 505.6962 | 372 | 439.9544 | 439.0897 | −2.01E−01 | 4.44E−01 | 1 | Unchanged Low | |
| Q9NQV6 | O22gh4 | PR domain containing 10 | 369.157 | 514 | 440.5347 | 441.3923 | 2.55E−01 | 4.79E−01 | 1 | Unchanged Low | |
| Q9NRX6 | J09gh4 | proteins × 013 | 324.5306 | 345 | 440.8995 | 370.1383 | 4.42E−01 | 8.82E−02 | 1 | Unchanged Low | |
| O76084 | A13cd4 | catenin (cadherin-associated pro | 471.1305 | 211 | 441.7617 | 374.6479 | −9.29E−02 | −1.16E+00 | 1 | Unchanged Low | |
| P23197 | E19cd7 | chromobox homolog 1 (HP1 beta | 480.8183 | 362 | 441.9595 | 428.3076 | −1.22E−01 | −4.09E−01 | 1 | Unchanged Low | |
| Q99439 | E10ab6 | calponin 2 | 429.1961 | 304 | 442.4148 | 391.978 | 4.38E−02 | 4.96E−01 | 1 ~Unchanged Low | | |
| O14656 | O04ef6 | dystonia 1; torsion (autosomal do | 415.3893 | 533 | 442.9429 | 463.6417 | 9.27E−02 | 3.59E−01 | 1 | Unchanged Low | |
| Q9NPJ3 | P04gh4 | uncharacterized hypothalamus p | 411.0811 | 430 | 443.2575 | 428.1718 | 1.09E−01 | 6.55E−02 | 1 | Unchanged Low | |
| P41226 | 810ef7 | ubiquitin-activating enzyme E1-lit | 298.6597 | 342 | 443.334 | 361.2685 | 5.70E−01 | 1.95E−01 | 1 | Unchanged Low | |
| Q9Y478 | C24cd1 | protein kinase: AMP-activated; b | 306.0158 | 165 | 443.5049 | 304.7576 | 5.35E−01 | −8.93E−01 | 1 | Unchanged Low | |
| | P06cd8 | nodal homolog (mouse) | 349.4202 | 252 | 443.7188 | 348.2928 | 3.45E−01 | 4.73E−01 | 1 | Unchanged Low | |
| Q12904 | O06e17 | small inducible cytokine subfamil | 374.6683 | 459 | 445.5171 | 426.5346 | 2.50E−01 | 2.94E−01 | 1 | Unchanged Low | |
| P20264 | C02cd1 | POU domain; class 3; transcription | 458.6994 | 302 | 447.8929 | 402.9521 | −3.44E−02 | −6.02E−01 | 1 | Unchanged Low | |
| Q15428 | B11cd4 | splicing factor 3a; subunit 2; 66kD | 264.0454 | 334 | 448.6652 | 355.7289 | 6.60E−01 | 2.36E−01 | 1 | Unchanged Low | |
| P49771 | G06ef6 | fms-related tyrosine kinase 3 liga | 449.6827 | 417 | 452.1329 | 439.4747 | 7.84E−03 | −1.10E−01 | 1 | Unchanged Low | |
| P49005 | A18cd1 | polymerase (DNA directed); delta | 363.5225 | 232 | 452.6832 | 349.3417 | 3.16E−01 | −6.49E−01 | 1 | Unchanged Low | |
| Q99879 | M14gh6 | H2B histone family; member E | 325.9523 | 483 | 452.8012 | 420.7323 | 4.74E−01 | 5.69E−01 | 1 | Unchanged Low | |
| P49821 | G20cd1 | NADH dehydrogenase (ubiquino | 511.5488 | 392 | 454.6223 | 452.5721 | −1.70E−01 | −3.86E−01 | 1 | Unchanged Low | |
| P10644 | H22ef5 | protein kinase: cAMP-dependent | 420.8225 | 518 | 454.8256 | 464.5909 | 1.12E−01 | 3.00E−01 | 1 | Unchanged Low | |
| Q9Y5J4 | L17gh6 | pyrroline 5-carboxylate reductase | 145.6987 | 303 | 455.1667 | 301.448 | 1.64E+00 | 1.06E+00 | 1 | Unchanged Low | |
| P31040 | K02cd2 | succinate dehydrogenase comple | 671.3369 | 351 | 456.2212 | 492.7974 | −5.57E−01 | −9.36E−01 | 1 | Unchanged Low | |
| Q9UHA2 | F04ef8 | synovial sarcoma translocation g | 438.6951 | 480 | 456.469 | 458.4094 | 5.73E−02 | 1.30E−01 | 1 | Unchanged Low | |
| Q9UBF6 | C02ef3 | ring finger protein 7 | 315.6541 | 400 | 456.9368 | 390.7196 | 5.34E−01 | 3.40E−01 | 1 | Unchanged Low | |
| Q9Y5S2 | C21cd6 | CDC42 binding protein kinase be | 386.9683 | 447 | 456.9709 | 430.2759 | 2.40E−01 | 2.08E−01 | 1 | Unchanged Low | |
| P20809 | H07ef7 | interleukin 11 | 399.048 | 140 | 458.1544 | 332.3748 | 1.99E−01 | −1.51E+00 | 1 | Unchanged Low | |
| Q13526 | F21ef5 | protein (peptidyl-prolyl cis/trans i | 333.4194 | 460 | 458.2237 | 417.3666 | 4.59E−01 | 4.66E−01 | 1 | Unchanged Low | |
| Q9NWQ9 | H01gh2 | hypothetical protein FLJ20671 | 430.7122 | 577 | 458.2293 | 488.6936 | 8.93E−02 | 4.22E−01 | 1 | Unchanged Low | |
| Q9Y6J0 | E05cd8 | calcineurin binding protein 1 | 474.3964 | 117 | 458.7489 | 350.127 | −4.84E−02 | −2.02E+00 | 1 | Unchanged Low | |
| Q9Y4Z6 | B13cd7 | vacuolar protein sorting 45A (yea | 115.1679 | 276 | 458.8586 | 283.3006 | 1.99E+00 | 1.26E+00 | 1 | Unchanged Low | |
| Q01433 | G02ab2 | adenosine monophosphate dean | 352.5251 | 390 | 459.2935 | 400.6864 | 3.82E−01 | 1.47E−01 | 1 | Unchanged Low | |
| O43181 | M07ab8 | NADH dehydrogenase (ubiquinoi | 417.4586 | 432 | 460.2425 | 436.6589 | 1.41E−01 | 5.03E−02 | 1 | Unchanged Low | |
| Q9NWT5 | D15gh2 | hypothetical protein FLJ20618 | 354.3599 | 523 | 460.3326 | 445.8286 | 3.77E−01 | 5.61E−01 | 1 | Unchanged Low | |
| O75431 | P19cd6 | metaxin 2 | 282.4791 | 443 | 460.352 | 395.1258 | 7.05E−01 | 6.48E−01 | 1 | Unchanged Low | |
| P09543 | A18gh7 | 2′:3′-cyclic nucleotide | 258.2862 | 510 | 462.033 | 409.9513 | 8.39E−01 | 9.50E−01 | 1 | Unchanged Low | |
| | | 3′ phospho | | | | | | | | | |
| Q9BTE1 | B05gh8 | dynactin 4 | 562.6142 | 460 | 462.4054 | 494.9234 | −2.832−01 | −2.912−01 | 1 | Unchanged Low | |
| P16152 | H14ab5 | carbonyl reductase 1 | 234.3184 | 240 | 463.6916 | 312.5841 | 9.85E−01 | 3.302−02 | 1 | Unchanged Low | |
| O15121 | D06cd3 | degenerative spermatocyte homo | 264.7024 | 270 | 464.4612 | 333.1906 | 8.11E−01 | 3.082−02 | 1 | Unchanged Low | |
| O60909 | E09ab3 | UDP-Gal:betaGlcNAc beta 1;4- g | 365.8881 | 379 | 464.9697 | 403.197 | 3.46E−01 | 4.982−02 | 1 | Unchanged Low | |
| O75223 | C11gh6 | hypothelical protein MGC3077 | 493.9011 | 276 | 465.7709 | 411.9427 | −8.46E−02 | −5.392−01 | 1 | Unchanged Low | |
| Q9BQA1 | K03gh6 | MEP50 protein | 379.1943 | 303 | 466.1997 | 352.8783 | 2.98E−01 | −3.222−01 | 1 | Unchanged Low | |
| O43427 | F20cd4 | fibroblast growth factor (acidic) in | 323.6471 | 325 | 466.8724 | 371.8858 | 5.29E−01 | 6.632−03 | 1 | Unchanged Low | |
| Q9BWG6 | A15gh6 | hypothetical protein MGC3180 | 309.3486 | 596 | 468.0924 | 457.7257 | 5.98E−01 | 9.452−01 | 1 | Unchanged Low | |
| Q9Y6R2 | M04cd6 | chromosome 4 open reading fra | 337.7358 | 352 | 469.9502 | 386.6911 | 4.77E−01 | 6.132−02 | 1 | Unchanged Low | |
| Q9HBK7 | M07gh4 | NPD007 protein | 367.4893 | 489 | 471.1924 | 442.4205 | 3.59E−01 | 4.112−01 | 1 | Unchanged Low | |
| P52758 | E04ef7 | translational inhibitor protein p14 | 324.9333 | 318 | 471.2741 | 371.3427 | 5.36E−01 | −3.192−02 | 1 | Unchanged Low | |
| P47897 | E19cd1 | glutaminyl-tRNA synthetase | 400.1718 | 364 | 472.5359 | 412.2837 | 2.40E−01 | −1.362−01 | 1 | Unchanged Low | |
| Q9BWQ6 | P20gh5 | hypothetical protein MGC3262 | 288.8083 | 483 | 473.0084 | 415.0843 | 7.12E−01 | 7.432−01 | 1 | Unchanged Low | |
| P35325 | H22cd2 | small proline-rich protein 2B | 390.5307 | 584 | 474.2762 | 483.0992 | 2.50E−01 | 5.822−01 | 1 | Unchanged Low | |
| Q9HA68 | O23gh5 | hypothetical protein FLJ12154 | 556.3676 | 383 | 474.593 | 471.3877 | −2.29E−01 | −5.382−01 | 1 | Unchanged Low | |
| P49366 | P16ab5 | deoxyhypusine synthase | 458.6886 | 481 | 477.1999 | 472.2313 | 5.71E−02 | 6.792−02 | 1 | Unchanged Low | |
| Q9UKZ7 | J18ef1 | colon carcinoma related protein | 505.0182 | 450 | 478.4787 | 477.7496 | −7.79E−02 | −1.672−01 | 1 | Unchanged Low | |
| P01730 | K15ef5 | CD4 antigen (p55) | 516.4046 | 432 | 478.8033 | 475.788 | −1.09E−01 | −2.57E−01 | 1 | Unchanged Low | |
| P20674 | L06cd4 | cytochrome c oxidase subunit Va | 512.4055 | 372 | 480.2708 | 454.7764 | −9.34E−02 | −4.632−01 | 1 | Unchanged Low | |
| Q12828 | H02ef6 | far upstream element (FUSE) bin | 462.592 | 312 | 480.5079 | 415.2868 | 5.48E−02 | −5.69E−01 | 1 | Unchanged Low | |
| Q9Y6A4 | J18ef3 | similar to mouse Glt3 or D, malar | 359.9248 | 485 | 480.8661 | 442.0882 | 4.18E−01 | 4.32E−01 | 1 | Unchanged Low | |
| O60888 | B09ef2 | divalent cation toterant protein C | 298.6981 | 304 | 481.136 | 361.3342 | 6.88E−01 | 2.62E−02 | 1 | Unchanged Low | |
| O75716 | F08cd3 | serine/threonine kinase 16 | 364.1875 | 379 | 481.1652 | 408.0382 | 4.02E−01 | 5.66E−02 | 1 | Unchanged Low | |
| P30711 | E18ef7 | glutathione S-transferase theta 1 | 327.8121 | 533 | 481.9461 | 447.6268 | 5.56E−01 | 7.02E−01 | 1 | Unchanged Low | |
| Q15008 | L03gh1 | KIAA0107 gene product | 475.6485 | 506 | 482.3327 | 488.0851 | 2.01E−02 | 9.00E−02 | 1 | Unchanged Low | |
| P17174 | N07ab4 | glutamic-oxaloacetic transaminas | 412.5293 | 224 | 484.4384 | 373.5229 | 2.32E−01 | −8.84E−01 | 1 | Unchanged Low | |
| P13798 | K16ab2 | N-acylaminoacyl-peptide hydrola | 365.1218 | 351 | 484.44 | 400.3314 | 4.08E−01 | −5.51E−02 | 1 | Unchanged Low | |
| Q9P2R8 | E15ef4 | Misshapen/NIK-related kinase | 469.5725 | 511 | 484.515 | 488.4865 | 4.52E−02 | 1.23E−01 | 1 | Unchanged Low | |
| Q9UNT1 | E20cd7 | RAB; member of RAS oncogene | 268.916 | 347 | 484.9919 | 386.9643 | 8.51E−01 | 3.68E−01 | 1 | Unchanged Low | |
| O75607 | L11cd7 | nucleophosmin/nucleoplasmin; 3 | 350.2387 | 272 | 485.5671 | 369.1972 | 4.71E−01 | −3.66E−01 | 1 | Unchanged Low | |
| O94925 | B20gh1 | glutaminase | 410.4357 | 471 | 486.8946 | 456.0458 | 2.46E−01 | 1.98E−01 | 1 | Unchanged Low | |
| O95390 | A06ab3 | growth differentiation factor 11 | 264.606 | 324 | 489.3912 | 359.3416 | 8.87E−01 | 2.92E−01 | 1 | Unchanged Low | |
| O75956 | K13cd6 | tumor suppressor deteled in oral | 524.962 | 482 | 489.6212 | 498.9664 | −1.01E−01 | −1.22E−01 | 1 | Unchanged Low | |
| O95573 | I23ab5 | fatty-acid-Coenzyme A ligase; lo | 354.1387 | 174 | 489.6455 | 339.2885 | 4.67E−01 | −1.02E+00 | 1 Unchanged Low | | |
| Q9BXH1 | N23gh6 | Bcl-2 binding component 3 | 493.3887 | 433 | 492.1083 | 472.8963 | −3.75E−03 | −1.88E−01 | 1 | Unchanged Low | |
| P13662 | L20cd5 | nuclear transport factor 2 | 392.576 | 479 | 492.4942 | 454.7336 | 3.27E−01 | 2.87E−01 | 1 | Unchanged Low | |
| Q92793 | H05ab5 | CREB binding protein (Rubinstei | 468.559 | 474 | 494.3525 | 478.8243 | 7.73E−02 | 1.53E−02 | 1 | Unchanged Low | |
| Q9P0H6 | K23ef4 | AD-012 protein | 408.4789 | 469 | 495.1858 | 456.7944 | 2.85E−01 | 2.06E−01 | 1 | Unchanged Low | |
| Q9P0R3 | C05ef3 | hypothetical protein HSPC213 | 339.0916 | 509 | 495.2656 | 447.9021 | 5.47E−01 | 5.57E−01 | 1 | Unchanged Low | |
| O75094 | E11gh1 | slit homolog 3 (Drosophila) | 345.9855 | 439 | 496.3612 | 426.952 | 5.21E−01 | 3.42E−01 | 1 | Unchanged Low | |
| Q9P011 | P10ef2 | non-canonical ubquitin conjugatin | 460.4398 | 538 | 497.256 | 498.5084 | 1.11E−01 | 2.24E−01 | 1 | Unchanged Low | |
| Q92504 | M12cd4 | HLA ctasa II region expressed ge | 251.7256 | 339 | 497.6138 | 362.6669 | 9.83E−01 | 4.28E−01 | 1 | Unchanged Low | |
| O95190 | L12cd8 | ornithine decarboxylase antizyme | 421.0592 | 441 | 497.6839 | 453.1247 | 2.41E−01 | 6.55E−02 | 1 | Unchanged Low | |
| Q9Y230 | N12cd6 | RuvB-like 2 (E. coli) | 342.0841 | 300 | 497.7767 | 379.9805 | 5.41E−01 | −1.89E−01 | 1 | Unchanged Low | |
| Q9BSG0 | M16gh8 | chromosome 2 open reading fran | 324.5321 | 458 | 497.8462 | 426.9191 | 6.17E−01 | 4.98E−01 | 1 | Unchanged Low | |
| Q9Y366 | I17ef2 | chromosome 20 open reading fra | 278.1968 | 453 | 498.3057 | 409.8539 | 8.41E−01 | 7.04E−01 | 1 | Unchanged Low | |
| Q14094 | O24ef5 | cyclin I | 407.6242 | 354 | 499.6223 | 420.31 | 2.94E−01 | −2.05E−01 | 1 | Unchanged Low | |
| P43403 | H04ef5 | zeta-chain (TCR) associated pro | 396.0583 | 396 | 500.6282 | 430.7684 | 3.38E−01 | −1.60E−03 | 1 | Unchanged Low | |
| P52803 | H23ef7 | ephrin-A5 | 357.5339 | 316 | 501.0718 | 391.4643 | 4.87E−01 | −1.79E−01 | 1 | Unchanged Low | |
| O95402 | E18cd5 | cofactor required for Sp1 transcri | 106.0683 | 58.7 | 501.9814 | 222.265 | 2.24E+00 | −8.52E−01 | 1 | Unchanged Low | |
| O00233 | L23ab8 | proteasome (prosome: macropai | 581.8818 | 399 | 502.2082 | 494.287 | −2.12E−01 | −5.45E−01 | 1 | Unchanged Low | |
| Q9H1K6 | O18gh7 | mesoderm development candida | 432.735 | 560 | 502.7516 | 498.6205 | 2.16E−01 | 3.73E−01 | 1 | Unchanged Low | |
| Q9BXL6 | K15gh6 | caspase recruitment domain prot | 425.1251 | 523 | 502.8514 | 483.629 | 2.42E−01 | 2.99E−01 | 1 | Unchanged Low | |
| Q9H173 | H03gh5 | endoplasmic recruitment domain prot | 456.5646 | 449 | 503.5156 | 469.6418 | 1.41E−01 | −2.46E−02 | 1 | Unchanged Low | |
| Q9NV83 | H03gh3 | hypothetical protein FLJ10876 | 423.9423 | 497 | 504.9702 | 475.4325 | 2.52E−01 | 2.30E−01 | 1 | Unchanged Low | |
| P11926 | A12ab8 | ornithine decarboxylase 1 | 591.3922 | 362 | 505.3016 | 486.3355 | −2.27E−01 | −7.07E−01 | 1 | Unchanged Low | |
| Q04743 | D23ef1 | empty spiracles homolog 2 (Dros | 531.3866 | 311 | 505.7367 | 449.3322 | −7.14E−02 | −7.73E−01 | 1 | Unchanged Low | |
| Q9BVK2 | G11gh6 | hypothetical protein MGC2840 si | 388.8682 | 383 | 505.9804 | 425.9595 | 3.80E−01 | −2.18E−02 | 1 | Unchanged Low | |
| O43251 | I16ef3 | RNA binding motif protein 9 | 346.6445 | 530 | 507.0674 | 461.1452 | 5.49E−01 | 6.12E−01 | 1 | Unchanged Low | |
| Q99829 | Ll3cd4 | copine I | 355.3783 | 268 | 508.4915 | 377.1688 | 5.17E−01 | −4.09E−01 | 1 | Unchanged Low | |
| O95777 | B10ef2 | U6 snRNA-associated Sm-like pr | 391.981 | 311 | 508.8683 | 403.8183 | 3.77E−01 | −3.36E−01 | 1 | Unchanged Low | |
| Q9UN53 | C19ef2 | calcium binding protein Cab45 p | 327.721 | 407 | 509.3316 | 414.6963 | 6.36E−01 | 3.13E−01 | 1 | Unchanged Low | |
| P24310 | F03ab4 | cytochrome c oxidase subunit VII | 184.0695 | 288 | 509.4504 | 327.1972 | 1.47E+00 | 6.46E−01 | 1 | Unchanged Low | |
| Q9H929 | N09gh5 | hypothetical protein FLJ13055 | 387.341 | 500 | 510.1197 | 465.7217 | 3.97E−01 | 3.67E−01 | 1 | Unchanged Low | |
| P10768 | K19ef1 | esterase D/formylglutathione hyd | 596.3068 | 102 | 510.5002 | 402.8142 | −2.24E−01 | −2.55E+00 | 1 | Unchanged Low | |
| O95287 | D17cd5 | golgi autoantigen; golgin subfam | 346.1135 | 560 | 510.6433 | 472.3714 | 5.61E−01 | 6.95E−01 | 1 | Unchanged Low | |
| Q01105 | H18cd1 | SET translocation (myeloid leuke | 328.6513 | 403 | 511.1477 | 414.2142 | 6.37E−01 | 2.94E−01 | 1 | Unchanged Low | |
| Q9UK45 | B08ef2 | U6 snRNA-associated Sm-like pr | 388.4486 | 551 | 511.9386 | 483.7088 | 3.98E−01 | 5.04E−01 | 1 | Unchanged Low | |
| P35611 | I17ab2 | adducin 1 (alpha) | 435.0349 | 519 | 512.6447 | 488.7819 | 2.37E−01 | 2.54E−01 | 1 | Unchanged Low | |
| O14681 | K22cd5 | etoposide-induced mRNA | 451.5599 | 324 | 516.5292 | 430.8211 | 1.94E−01 | −4.77E−01 | 1 | Unchanged Low | |
| P80303 | A05cd1 | nucleobindin 2 | 296.1418 | 236 | 516.7037 | 349.5918 | 8.03E−01 | −3.28E−01 | 1 | Unchanged Low | |
| Q9NWC0 | N19gh2 | F-box only protein 4 | 434.5394 | 477 | 517.7728 | 476.3476 | 2.53E−01 | 1.34E−01 | 1 | Unchanged Low | |
| P00367 | B06ab5 | glutamate dehydrogenase 1 | 360.6 | 303 | 518.2525 | 394.0342 | 5.23E−01 | −2.50E−01 | 1 | Unchanged Low | |
| O00469 | I09ab8 | procollagen-lysine: 2-oxoglutarat | 735.8327 | 238 | 518.6446 | 497.4006 | −5.05E−01 | −1.63E+00 | 1 | Unchanged Low | |
| Q9UI04 | M18ef4 | mitochondrial ribosomal protein | 364.1383 | 336 | 520.377 | 406.7523 | 5.15E−01 | −1.17E−01 | 1 | Unchanged Low | |
| P20338 | N08ab8 | RAB4A; member RAS oncogene | 487.9427 | 457 | 520.7608 | 488.5317 | 9.39E−02 | −9.49E−02 | 1 | Unchanged Low | |
| Q9HCU8 | I19gh4 | polymerase (DNA-directed): delta | 513.52 | 431 | 522.5153 | 489.0161 | 2.51E−02 | −2.53E−01 | 1 | Unchanged Low | |
| P00519 | I06ef5 | v-abl Abelson murine leukemia v | 232.4636 | 301 | 522.7624 | 352.0574 | 1.17E+00 | 3.73E−01 | 1 | Unchanged Low | |
| Q13880 | O02cd5 | brain and reproductive organ-exp | 572.4849 | 263 | 523.9906 | 453.0588 | −1.28E−01 | −1.12E+00 | 1 | Unchanged Low | |
| P25789 | L13ef7 | proteasome (prosome; macropai | 436.5895 | 534 | 526.397 | 498.9408 | 2.72E−01 | 2.90E−01 | 1 | Unchanged Low | |
| O43805 | J16cd3 | Sjogren's syndrome nuclear auto | 368.0123 | 539 | 526.5215 | 477.7167 | 5.17E−01 | 5.50E−01 | 1 | Unchanged Low | |
| O75817 | A13cd6 | POP7 (processing of precursor; S | 375.1187 | 433 | 526.6432 | 445.0255 | 4.89E−01 | 2.08E−01 | 1 | Unchanged Low | |
| Q9UBI1 | N11cd7 | BUP protein | 337.1018 | 428 | 526.9646 | 430.701 | 6.45E−01 | 3.45E−01 | 1 | Unchanged Low | |
| P03950 | G12ab2 | angiogenin; ribonuclease; RNase | 395.0198 | 200 | 527.7882 | 374.4251 | 4.18E−01 | −9.79E−01 | 1 | Unchanged Low | |
| P78423 | F20cd1 | small inducible cytokine subfamil | 201.965 | 747 | 530.2357 | 493.2272 | 1.39E+00 | 1.89E+00 | 1 | Unchanged Low | |
| Q9P1D0 | E07gh4 | hypothetical protein PRO2533 | 58.04051 | 73.3 | 531.5331 | 220.9467 | 3.20E+00 | 3.36E−01 | 1 | Unchanged Low | |
| P03999 | K05ab3 | opsin 1 (cone pigments); short-w | 116.5093 | 37.9 | 532.8048 | 229.0786 | 2.19E+00 | −1.62E+00 | 1 | Unchanged Low | |
| O95793 | M22cd2 | staufen; RNA binding protein (D | 417.7687 | 498 | 534.8626 | 483.6536 | 3.56E−01 | 2.54E−01 | 1 | Unchanged Low | |
| P22750 | M05ef2 | RAB4B; member RAS oncogene | 297.3584 | 346 | 538.4665 | 393.9782 | 8.57E−01 | 2.19E−01 | 1 | Unchanged Low | |
| | B06gh8 | ubiquitin UBF-fl | 431.0731 | 472 | 540.8482 | 481.1517 | 3.27E−01 | 1.29E−01 | 1 | Unchanged Low | |
| Q9NRX8 | J15gh4 | oxidoreductase UCPA | 168.7047 | 523 | 544.0864 | 411.9671 | 1.69E+00 | 1.63E+00 | 1 | Unchanged Low | |
| Q9UHQ3 | J20gh4 | stromal cell protein | 469.8784 | 214 | 546.1893 | 410.1503 | 2.17E−01 | −1.13E+00 | 1 | Unchanged Low | |
| Q96S52 | J22gh8 | phosphatidylinositol glycan: class | 404.9498 | 287 | 546.8906 | 412.8095 | 4.34E−01 | −4.99E−01 | 1 | Unchanged Low | |
| Q9H3F6 | C24gh8 | MSTP028 protein | 442.9007 | 396 | 549.0707 | 462.5636 | 3.10E−01 | −1.63E−01 | 1 | Unchanged Low | |
| P37198 | K03cd8 | nucleoporin 62kD | 428.2961 | 499 | 550.8083 | 492.7531 | 3.63E−01 | 2.21E−01 | 1 | Unchanged Low | |
| Q9H1D4 | B05gh4 | arginyl aminopeptidase (aminope | 430.3273 | 477 | 551.8541 | 486.2744 | 3.59E−01 | 1.47E−01 | 1 | Unchanged Low | |
| O75350 | K04cd5 | glycoprotein; synaptic 2 | 480.7082 | 436 | 552.8489 | 489.9278 | 2.02E−01 | −1.40E−01 | 1 | Unchanged Low | |
| Q9P024 | D16ef3 | Huntingtin interacting protein K | 369.5132 | 480 | 553.5493 | 467.714 | 5.53E−01 | 3.78E−01 | 1 | Unchanged Low | |
| Q9Y224 | H21ef2 | CGI-99 protein | 449.089 | 472 | 554.6607 | 492.0453 | 3.05E−01 | 7.30E−02 | 1 | Unchanged Low | |
| Q13361 | O12cd3 | Microfibril-associated glycoprotei | 194.28 | 354 | 557.066 | 368.553 | 1.52E+00 | 8.67E−01 | 1 | Unchanged Low | |
| Q02535 | F01ef1 | inhibitor of DNA binding 3; domin | 84.05787 | 174 | 559.513 | 272.6788 | 2.73E+00 | 1.05E+00 | 1 | Unchanged Low | |
| O95864 | L24cd4 | fatty acid desaturase 2 | 298.4502 | 113 | 559.6571 | 323.8122 | 9.07E−01 | −1.40E+00 | 1 | Unchanged Low | |
| P53621 | H16ab4 | coatomer protein complex; subur | 409.2779 | 471 | 560.6313 | 480.1571 | 4.54E−01 | 2.01E−01 | 1 | Unchanged Low | |
| P35638 | N03ab4 | DNA-damage-inducible transcrip | 322.5942 | 318 | 565.361 | 401.9484 | 6.09E−01 | −2.12E−02 | 1 | Unchanged Low | |
| P22307 | D22cd1 | sterol carrier protein 2 | 354.3674 | 356 | 569.1558 | 426.421 | 6.64E−01 | 5.56E−03 | 1 | Unchanged Low | |
| Q92543 | D15gh1 | KIAA0254 gene product | 328.3511 | 356 | 569.5472 | 417.8592 | 7.95E−01 | 1.15E−01 | 1 | Unchanged Low | |
| P78330 | N06ab8 | phosphoserine phosphatase | 253.4253 | 220 | 569.9825 | 347.8406 | 1.17E+00 | −2.03E−01 | 1 | Unchanged Low | |
| Q9UNX3 | G15ef2 | ribosomal protein L26-like 1 | 401.3697 | 445 | 572.3441 | 472.8205 | 5.12E−01 | 1.48E−01 | 1 | Unchanged Low | |
| P49189 | C10ab2 | aldehyde dehydrogenase 9 famil | 446.252 | 277 | 579.314 | 434.2707 | 3.76E−01 | −6.87E−01 | 1 | Unchanged Low | |
| Q9ULX0 | H03gh6 | inorganic pyrophosphatase | 488.3446 | 402 | 579.6142 | 489.9626 | 2.47E−01 | −2.81E−01 | 1 | Unchanged Low | |
| O15353 | H21cd3 | winged-helix nude | 407.9375 | 497 | 580.6191 | 495.2366 | 5.09E−01 | 2.85E−01 | 1 | Unchanged Low | |
| Q9NRP2 | D21gh4 | DC13 protein | 382.8544 | 421 | 561.5421 | 461.731 | 6.03E−01 | 1.36E−01 | 1 | Unchanged Low | |
| Q16280 | E18ef1 | cyclic nucleotide gated channel | 464.0608 | 360 | 564.8035 | 469.6748 | 3.34E−01 | −3.66E−01 | 1 | Unchanged Low | |
| P04183 | C04cd2 | thymidine kinase 1: soluble | 346.7317 | 289 | 566.3101 | 407.8979 | 7.63E−01 | −2.64E−01 | 1 | Unchanged Low | |
| Q12824 | O17ef5 | SWI/SNF related; matrix associa | 218.8831 | 554 | 590.2326 | 454.3734 | 1.43E+00 | 1.34E+00 | 1 | Unchanged Low | |
| Q99719 | B09ab8 | peanut-like 1 (Drosophila) | 419.5269 | 410 | 592.2598 | 474.0479 | 4.97E−01 | −3.19E−02 | 1 | Unchanged Low | |
| P46100 | H07ab3 | alpha thalassemia/mental retard | 309.8648 | 167 | 594.3041 | 356.9963 | 9.40E−01 | −6.93E−01 | 1 | Unchanged Low | |
| Q16610 | O05ab5 | extracellular matrix protein 1 | 238.3172 | 335 | 596.1691 | 359.6774 | 1.32E+00 | 4.69E−01 | 1 | Unchanged Low | |
| P39656 | E23ab6 | dolichyl-diphosphooligosacchari | 433.6719 | 367 | 596.0666 | 466.4028 | 4.64E−01 | −2.39E−01 | 1 | Unchanged Low | |
| P53814 | G07cd3 | smoothetin | 517.4102 | 269 | 596.16 | 461.5158 | 2.09E−01 | −9.44E−01 | 1 | Unchanged Low | |
| Q9P0N4 | P24ef1 | hypothetical protein LOC51260 | 609.9142 | 286 | 601.9324 | 499.258 | −1.90E−02 | −1.09E+00 | 1 | Unchanged Low | |
| Q9BXZ1 | A02gh7 | peptidylprolyl isomerase (cyclop | 465.4301 | 362 | 603.8737 | 477.1695 | 3.76E−01 | −3.62E−01 | 1 | Unchanged Low | |
| Q9Y2Q4 | H19ef8 | HSPC002 protein | 337.5615 | 391 | 604.9187 | 444.641 | 8.42E−01 | 2.14E−01 | 1 | Unchanged Low | |
| O15527 | A18ab8 | 8-oxoguanine DNA glycosylase | 330.4852 | 282 | 605.3444 | 406.0683 | 8.73E−01 | −2.27E−01 | 1 | Unchanged Low | |
| Q04323 | G01ef2 | ORF | 497.159 | 255 | 610.1121 | 484.2076 | 2.95E−01 | −9.61E−01 | 1 | Unchanged Low | |
| P12004 | G18ab8 | proliferating cell nuclear antigen | 332.1465 | 303 | 612.821 | 416.0863 | 8.84E−01 | −1.31E−01 | 1 | Unchanged Low | |
| Q99720 | M11cd6 | sigma receptor (SR31747 binding | 363.6609 | 379 | 615.0729 | 452.6168 | 7.57E−01 | 5.85E−02 | 1 | Unchanged Low | |
| P43307 | G13cd2 | signal sequence receptor: alpha | 226.1566 | 322 | 618.9554 | 389.0352 | 1.45E+00 | 5.10E−01 | 1 | Unchanged Low | |
| Q9H3Y8 | O05gh6 | chromosome 20 open reading fra | 221.2984 | 246 | 619.4944 | 362.4108 | 1.49E+00 | 1.55E−01 | 1 | Unchanged Low | |
| Q9NRX2 | A08gh5 | mitochondrial ribosomal protein | 284.6282 | 305 | 627.9834 | 405.9846 | 1.14E+00 | 1.01E−01 | 1 | Unchanged Low | |
| Q9P2X0 | L10gh2 | dolichyl-phosphate mannosyltran | 285.542 | 431 | 638.6696 | 451.6107 | 1.16E+00 | 5.95E−01 | 1 | Unchanged Low | |
| Q9Y5Z8 | E05cd7 | male-specific lethal 3-like 1 (Dros | 361.7331 | 394 | 639.7431 | 465.0297 | 8.23E−01 | 1.22E−01 | 1 | Unchanged Low | |
| P78524 | F01cd2 | suppression of tumorigenicity 5 | 343.4117 | 289 | 843.333 | 425.1425 | 9.06E−01 | −2.50E−01 | 1 | Unchanged Low | |
| P55145 | F02ab2 | arginine-rich; mutated in early sta | 413.1582 | 420 | 656.6324 | 496.4892 | 6.68E−01 | 2.26E−02 | 1 | Unchanged Low | |
| P33551 | O23ab5 | CDC28 protein kinase 1 | 296.3533 | 355 | 671.4891 | 440.861 | 1.18E+00 | 2.59E−01 | 1 | Unchanged Low | |
| Q9BVQ0 | E09gh6 | hypothetical protein MGC5363 | 43.30788 | 54.5 | 681.2449 | 259.6855 | 3.98E+00 | 3.32E−01 | 1 | Unchanged Low | |
| P34897 | D15cd2 | serine hydroxymethyttransferase | 440.9652 | 290 | 685.6584 | 472.1838 | 6.37E−01 | −6.05E−01 | 1 | Unchanged Low | |
| Q9Y5B4 | L23ef2 | androgen induced protein | 268.4081 | 519 | 685.9395 | 491.1814 | 1.35E+00 | 9.52E−01 | 1 | Unchanged Low | |
| Q9Y2V5 | B15ef2 | transforming growth factor beta 1 | 249.1479 | 327 | 712.3879 | 429.636 | 1.52E+00 | 3.94E−01 | 1 | Unchanged Low | |
| Q13953 | B19cd2 | solute carrier family 12 (potassiu | 342.5865 | 428 | 729.2688 | 499.9658 | 1.09E+00 | 3.21E−01 | 1 | Unchanged Low | |
| Q92686 | I11ef6 | neurogranin (protein kinase C su | 330.8792 | 299 | 736.2348 | 455.4495 | 1.15E+00 | −1.45E−01 | 1 | Unchanged Low | |
| O60565 | J05cd8 | cysteine knot superfamily 1; BMF | 106.6904 | 151 | 781.0154 | 346.3594 | 2.87E+00 | 5.05E−01 | 1 | Unchanged Low | |
| Q9Y4M4 | I21gh3 | putative UDP-GalNAc:polypeptid | 346.098 | 251 | 846.4309 | 481.2315 | 1.29E+00 | −4.63E−01 | 1 | Unchanged Low | |
| P16581 | E01ef7 | selectin E (endothelial adhesion | 109.3094 | 378 | 675.1666 | 454.0605 | 3.00E+00 | 1.79E+00 | 1 | Unchanged Low | |
| O14733 | P02ef5 | mitogen-activated protein kinase | 98.83312 | 140 | 888.014 | 375.5944 | 3.17E+00 | 5.02E−01 | 1 | Unchanged Low | |
| Q16623 | M24cd2 | syntaxin 1A (brain) | 403.2337 | 165 | 926.5022 | 498.3425 | 1.20E+00 | −1.29E+00 | 1 | Unchanged Low | |
| Q9P1M5 | P12gh3 | WW domain containing oxidored | 38.93 | 19.3 | 1292.23 | 450.1426 | 5.05E+00 | −1.01E+00 | 1 | Unchanged Low | |
| O60613 | L18cd4 | 15kD selenoprotein | 1220.902 | 1110 | 1285.07 | 1205.045 | 7.39E−02 | −1.38E−01 | 2 | Unchanged Medium | |
| Q16698 | M07ab4 | 2;4-dienoyl CoA reductase 1; mit | 469.9997 | 706 | 615.3087 | 597.2594 | 3.89E−01 | 5.86E−01 | 2 | Unchanged Medium | |
| Q9HBA8 | D23gh1 | 24-dehydrocholesterot reductase | 768.0888 | 278 | 626.0195 | 557.2677 | −2.95E−01 | −1.47E+00 | 2 | Unchanged Medium | |
| Q9P0I2 | M13ef4 | 30kDa protein | 546.352 | 691 | 614.5795 | 617.4113 | 1.70E−01 | 3.39E−01 | 2 | Unchanged Medium | |
| Q9UL53 | P24cd7 | 5′-3′exoribonuclease 2 | 648.132 | 604 | 430.2235 | 560.6322 | −5.91E−01 | −1.03E−01 | 2 | Unchanged Medium | |
| P49902 | N16cd7 | 5′-nucleotidase; cytosolic II | 1040.523 | 1220 | 440.0817 | 900.9404 | −1.24E+00 | 2.32E−01 | 2 | Unchanged Medium | |
| O95336 | P09cd7 | 6-phosphogluconolactonase | 524.3104 | 755 | 812.5955 | 697.4428 | 6.32E−01 | 5.27E−01 | 2 | Unchanged Medium | |
| Q03393 | A23ab8 | 6-pyruvoyltetrahydropterin synth | 358.0591 | 700 | 517.4355 | 525.0184 | 5.31E−01 | 9.66E−01 | 2 | Unchanged Medium | |
| Q13443 | P01ef7 | a disintegrin and metalloproteina | 1306.754 | 481 | 445.3663 | 744.5037 | −1.55E+00 | −1.44E+00 | 2 | Unchanged Medium | |
| O75173 | F15ab2 | a disintegrin-like and melalloprot | 786.5151 | 1060 | 1254.804 | 1033.446 | 6.74E−01 | 4.29E−01 | 2 | Unchanged Medium | |
| P51572 | O03gh1 | accessory proteins BAP31/BAP2 | 985.1381 | 889 | 656.6554 | 843.6943 | −5.85E−01 | −1.48E−01 | 2 | Unchanged Medium | |
| P42765 | D03ab2 | acetyl-Coenzyme A acyltransfera | 849.6041 | 1280 | 1204.131 | 1111.329 | 5.03E−01 | 5.92E−01 | 2 | Unchanged Medium | |
| P24666 | E05ab2 | acid phosphatase 1: soluble | 1642.966 | 1970 | 1890.909 | 1833.204 | 2.03E−01 | 2.59E−01 | 2 | Unchanged Medium | |
| Q92688 | D21cd6 | acidic (leucine-rich) nuclear phos | 470.8575 | 289 | 835.9103 | 531.9498 | 8.28E−01 | −7.04E−01 | 2 | Unchanged Medium | |
| O14639 | B21ab2 | actin binding LIM protein | 1351.973 | 393 | 356.779 | 701.2899 | −1.91E+00 | −1.78E+00 | 2 | Unchanged Medium | |
| O15143 | F06ab2 | actin related protein 2/3 complex | 696.0033 | 513 | 349.0474 | 519.3549 | −9.96E−01 | −4.40E−01 | 2 | Unchanged Medium | |
| O15509 | F12ab2 | actin related protein 2/3 complex | 909.0832 | 889 | 807.751 | 868.6913 | −1.71E−01 | −3.18E−02 | 2 | Unchanged Medium | |
| O15511 | F14ab2 | actin related protein 2/3 complex | 732.1996 | 619 | 468.1372 | 605.8842 | −6.51E−01 | −2.42E−01 | 2 | Unchanged Medium | |
| P12718 | A08ab6 | actin: gamma 2; smooth muscle; | 585.3438 | 846 | 2095.96 | 1175.856 | 1.84E+00 | 5.32E−01 | 2 | Unchanged Medium | |
| Q9P0I6 | O09ef4 | AD-015 protein | 947.0366 | 837 | 1259.571 | 1014.448 | 4.11E−01 | −1.79E−01 | 2 | Unchanged Medium | |
| O43747 | M13ab2 | adaptor-related protein complex | 1104.332 | 532 | 419.4712 | 685.2957 | −1.40E+00 | −1.05E+00 | 2 | Unchanged Medium | |
| P20172 | A11ab6 | adaptor-related protein complex: | 1286.83 | 1280 | 2285.193 | 1617.354 | 8.28E−01 | −7.63E−03 | 2 | Unchanged Medium | |
| O14617 | H07ab2 | adaptor-related protein complex: | 679.4847 | 495 | 357.0868 | 510.4994 | −9.28E−01 | −4.57E−01 | 2 | Unchanged Medium | |
| P07741 | M24ab2 | adenine phosphoribosyltransfera | 1885.065 | 862 | 823.8455 | 1190.468 | −1.19E+00 | −1.13E+00 | 2 | Unchanged Medium | |
| P00568 | A02ab2 | adenylate kinase 1 | 2404.119 | 399 | 354.3768 | 1052.361 | −2.76E+00 | −2.59E+00 | 2 | Unchanged Medium | |
| Q9UIJ7 | I06ef2 | adenylate kinase 3 alpha like | 758.7288 | 1060 | 662.8834 | 828.3999 | −1.95E−01 | 4.87E−01 | 2 | Unchanged Medium | |
| P30566 | M07ab2 | adenylosuccinate lyase | 495.8874 | 622 | 518.4238 | 545.4304 | 6.41E−02 | 3.27E−01 | 2 | Unchanged Medium | |
| Q16186 | M11cd7 | adhesion regulating molecule 1 | 622.3901 | 610 | 574.5888 | 602.2578 | −1.15E−01 | −2.95E−02 | 2 | Unchanged Medium | |
| Q9NVF6 | B19gh3 | ADP-ribosylation factor 1 GTPas | 449.3679 | 623 | 676.1878 | 582.9297 | 5.90E−01 | 4.72E−01 | 2 | Unchanged Medium | |
| P26438 | B04ab2 | ADP-ribosylation factor 6 | 732.1241 | 818 | 876.9277 | 809.1342 | 2.60E−01 | 1.61E−01 | 2 | Unchanged Medium | |
| P40616 | D12ab2 | ADP-ribosylation factor-like 1 | 827.1898 | 1220 | 1141.92 | 1084.016 | 4.65E−01 | 5.64E−01 | 2 | Unchanged Medium | |
| P36404 | D14ab2 | ADP-ribosylation factor-like 2 | 1751.048 | 1550 | 1871.661 | 1723.74 | 9.61E−02 | −1.77E−01 | 2 | Unchanged Medium | |
| P56559 | D20ab2 | ADP-ribosylation factor-like 7 | 485.4562 | 598 | 568.6824 | 550.5764 | 2.28E−01 | 3.00E−01 | 2 | Unchanged Medium | |
| P35368 | K17ab2 | adrenergic: alpha-1B-; receptor | 1433.715 | 1510 | 1335.995 | 1426.069 | −1.02E−01 | 7.34E−02 | 2 | Unchanged Medium | |
| P15144 | G24ab2 | alanyl (membrane) aminopeptida | 987.3343 | 222 | 480.1162 | 563.2289 | −1.04E+00 | −2.15E+00 | 2 | Unchanged Medium | |
| P30038 | L09ab2 | aldehyde dehydrogenase 4 famil | 1179.797 | 598 | 657.4315 | 811.7082 | −8.44E−01 | −9.81E−01 | 2 | Unchanged Medium | |
| P14550 | L05ab2 | aldo-keto reductase family 7; me | 555.464 | 886 | 609.9901 | 683.9189 | 1.35E−01 | 6.74E−01 | 2 | Unchanged Medium | |
| O43488 | L07ab2 | aldo-keto reductase family 7; me | 546.409 | 525 | 748.5424 | 606.6855 | 4.54E−01 | −5.74E−02 | 2 | Unchanged Medium | |
| P09972 | C16ab2 | aldolase C; fructose-bisphosphat | 878.5714 | 1050 | 947.5062 | 960.156 | 1.09E−01 | 2.63E−01 | 2 | Unchanged Medium | |
| O43590 | G12ef4 | alpha-actinin-2-associated LIM p | 776.3757 | 1070 | 501.1993 | 783.883 | −6.31E−01 | 4.68E−01 | 2 | Unchanged Medium | |
| Q08117 | M19ab2 | amino-terminal enhancer of split | 1779.707 | 1430 | 1219.695 | 1476.318 | −5.45E−01 | −3.16E−01 | 2 | Unchanged Medium | |
| Q13438 | G05cd7 | amplified in osteosarcoma | 658.0513 | 638 | 802.3407 | 699.6136 | 2.86E−01 | −4.36E−02 | 2 | Unchanged Medium | |
| O96018 | M01gh1 | amyloid beta (A4) precursor prot | 762.3898 | 750 | 1117.682 | 876.6611 | 5.52E−01 | −2.36E−02 | 2 | Unchanged Medium | |
| Q13564 | P11ab2 | amyloid beta precursor protein bi | 587.5144 | 664 | 786.6426 | 679.5497 | 4.21E−01 | 1.78E−01 | 2 | Unchanged Medium | |
| Q9Y679 | B08cd7 | ancient ubiquitous protein 1 | 1191.741 | 1270 | 1022.771 | 1160.442 | −2.21E−01 | 6.61E−02 | 2 | Unchanged Medium | |
| Q9UKB4 | B12ef2 | angiomotin like 2 | 523.7253 | 776 | 566.8766 | 622.7291 | 1.19E−01 | 5.66E−01 | 2 | Unchanged Medium | |
| Q13725 | H19ab2 | angiotensin receptor 1 | 1495.332 | 1360 | 1056.942 | 1304.886 | −4.96E−01 | −1.36E−01 | 2 | Unchanged Medium | |
| P08133 | I20ab2 | annexin A6 | 253.0936 | 413 | 879.9474 | 515.5041 | 1.60E+00 | 7.08E−01 | 2 | Unchanged Medium | |
| P20073 | I22ab2 | annexin A7 | 650.1591 | 481 | 420.4945 | 517.0979 | −6.29E−01 | −4.36E−01 | 2 | Unchanged Medium | |
| P03971 | E22ab2 | anti-Mullerian hormone | 1140.812 | 948 | 640.7158 | 909.9247 | −8.32E−01 | −2.67E−01 | 2 | Unchanged Medium | |
| P30041 | O08cd5 | anti-oxidant protein 2 (non-seteni | 1248.922 | 1160 | 3067.945 | 1824.225 | 1.30E+00 | −1.12E−01 | 2 | Unchanged Medium | |
| P27695 | K06ef6 | APEX nuclease (multifunctional | 665.9854 | 653 | 835.6897 | 718.2149 | 3.27E−01 | −2.85E−02 | 2 | Unchanged Medium | |
| Q9Y4J7 | P01ab2 | apoptosis inhibitor 5 | 1850.534 | 68.4 | 147.4186 | 688.7802 | −3.65E+00 | −4.76E+00 | 2 | Unchanged Medium | |
| Q9Y5L7 | L03ef2 | apoptosis related protein APR-3 | 662.9122 | 583 | 1426.489 | 890.7729 | 1.11E+00 | −1.86E−01 | 2 | Unchanged Medium | |
| O94778 | O14ab2 | aquaporin 8 | 1475.036 | 1420 | 1474.307 | 1455.01 | −7.13E−04 | −5.92E−02 | 2 | Unchanged Medium | |
| O15296 | E02ab2 | arachidonate 15-lipoxygenase: s | 826.8209 | 607 | 720.2105 | 718.1334 | −1.99E−01 | −4.45E−01 | 2 | Unchanged Medium | |
| P48444 | O18ab2 | archain 1 | 1291.871 | 1200 | 1374.223 | 1288.649 | 8.92E−02 | −1.07E−01 | 2 | Unchanged Medium | |
| P00966 | J24ab2 | argininosuccinate synthetase | 1049.688 | 1600 | 949.4889 | 1199.548 | −1.45E−01 | 6.08E−01 | 2 | Unchanged Medium | |
| P42024 | D15ab2 | ARP1 actin-related protein 1 hom | 1054.286 | 1720 | 1777.944 | 1518.804 | 7.54E−01 | 7.10E−01 | 2 | Unchanged Medium | |
| O43681 | J16ab2 | arsA arsenite transporter; ATP-bi | 540.3448 | 724 | 595.3348 | 620.0089 | 1.40E−01 | 4.23E−01 | 2 | Unchanged Medium | |
| Q9ULX3 | E02ef4 | ART-4 protein | 728.7274 | 595 | 509.3934 | 611.0757 | −5.17E−01 | −2.92E−01 | 2 | Unchanged Medium | |
| O96030 | H16ab2 | artemin | 881.7849 | 728 | 636.2016 | 749.363 | −4.66E−01 | −2.76E−01 | 2 | Unchanged Medium | |
| P15289 | F24ab2 | arylsulfatase A | 1639.546 | 1230 | 1012.121 | 1294.172 | −6.96E−01 | −4.14E−01 | 2 | Unchanged Medium | |
| P08243 | J18ab2 | asparagine synthetase | 891.3027 | 643 | 1540.113 | 1024.719 | 7.89E−01 | −4.72E−01 | 2 | Unchanged Medium | |
| O95630 | L17ab2 | associated molecule with the SH | 660.3522 | 872 | 729.2383 | 753.7548 | 1.43E−01 | 4.01E−01 | 2 | Unchanged Medium | |
| O95135 | B09ab2 | ataxin 2 related protein | 902.4529 | 345 | 342.3299 | 530.0852 | −1.40E+00 | −1.39E+00 | 2 | Unchanged Medium | |
| O14530 | N15ab2 | ATP binding protein associated v | 827.4646 | 1390 | 766.257 | 993.482 | −1.11E−01 | 7.45E−01 | 2 | Unchanged Medium | |
| P30049 | N20ab2 | ATP synthase: H+ transporting: | 625.0408 | 468 | 441.4691 | 511.6658 | −5.02E−01 | −4.16E−01 | 2 | Unchanged Medium | |
| Q9UII2 | I10ef2 | ATPase inhibitor precursor | 776.4612 | 1220 | 641.2787 | 878.5341 | −2.76E−01 | 6.49E−01 | 2 | Unchanged Medium | |
| Q9Y5K8 | F17ef2 | ATPase: H+ transporting: lysoso | 998.0597 | 1670 | 1029.866 | 1232.91 | 4.53E−02 | 7.43E−01 | 2 | Unchanged Medium | |
| O75787 | P15ab2 | ATPase: H+ transporting; lysoso | 815.707 | 945 | 1081.688 | 947.5842 | 4.07E−01 | 2.13E−01 | 2 | Unchanged Medium | |
| P05023 | P09ef5 | ATPase: Na+/K+ transporting: al | 1299.236 | 517 | 709.0882 | 841.8422 | −8.74E−01 | −1.33E+00 | 2 | Unchanged Medium | |
| P45844 | P13ef5 | ATP-binding cassette: sub-family | 863.8068 | 381 | 606.1642 | 616.8957 | −5.11E−01 | −1.18E+00 | 2 | Unchanged Medium | |
| Q9HAP7 | M08gh5 | baculoviral IAP repeat-containing | 1507.175 | 1740 | 1945.196 | 1730.54 | 3.66E−01 | 2.07E−01 | 2 | Unchanged Medium | |
| O94812 | G09ab3 | BAI1-associated protein 3 | 1073.67 | 983 | 826.6524 | 961.2437 | −3.77E−01 | −1.27E−01 | 2 | Unchanged Medium | |
| P35613 | B14ef7 | basigin (OK blood group) | 603.4821 | 474 | 865.2409 | 647.414 | 5.20E−01 | −3.50E−01 | 2 | Unchanged Medium | |
| Q9BSR6 | A20gh8 | BBP-like protein 1 | 816.219 | 1320 | 914.1927 | 1015.173 | 1.64E−01 | 6.88E−01 | 2 | Unchanged Medium | |
| O00512 | K01ab3 | B-cell CLL/lymphoma 9 | 1357.15 | 76.7 | 80.36541 | 504.741 | −4.06E+00 | −4.15E+00 | 2 | Unchanged Medium | |
| Q12983 | E19ab5 | BCL2/adenovirus E1B 19kD inte | 763.7046 | 548 | 991.4529 | 767.5569 | 3.77E−01 | 4.80E−01 | 2 | Unchanged Medium | |
| Q92934 | K24ef1 | BCL2-antagonist of cell death | 538.8191 | 727 | 603.5103 | 623.2249 | 1.64E−01 | 4.33E−01 | 2 | Unchanged Medium | |
| Q07817 | E10ef6 | BCL2-like 1 | 2062.673 | 2510 | 1097.372 | 1890.905 | −9.10E−01 | 2.85E−01 | 2 | Unchanged Medium | |
| O43892 | M23ab3 | Bicaudal D homolog 1 (Drosophil | 654.9397 | 414 | 548.7409 | 539.1375 | −2.55E−01 | −6.63E−01 | 2 | Unchanged Medium | |
| P53004 | O19ab3 | biliverdin reductase A | 490.0724 | 569 | 512.2807 | 523.696 | 6.39E−02 | 2.15E−01 | 2 | Unchanged Medium | |
| O60629 | G21ab3 | bladder cancer associated protei | 607.9847 | 1180 | 1083.411 | 956.1289 | 8.33E−01 | 9.53E−01 | 2 | Unchanged Medium | |
| Q9HBX3 | N21gh6 | brain and nasopharyngeal carcin | 1672.876 | 54 | 44.02379 | 590.2997 | −5.25E+00 | −4.95E+00 | 2 | Unchanged Medium | |
| P56945 | H23ef3 | breast cancer anti-estrogen resis | 1334.88 | 1300 | 1304.356 | 1312.13 | −3.34E−02 | −4.14E−02 | 2 | Unchanged Medium | |
| O15255 | G21cd6 | CAAX box 1 | 733.6878 | 577 | 1202.182 | 837.48 | 7.12E−01 | −3.48E−01 | 2 | Unchanged Medium | |
| P12830 | C16ef5 | cadherin 1: type 1; E-cadherin (e | 1094.64 | 924 | 489.5012 | 835.8959 | −1.16E+00 | −2.45E−01 | 2 | Unchanged Medium | |
| P19022 | E03ef7 | cadherin 2: type 1; N-cadherin (n | 337.5528 | 540 | 747.1198 | 541.7151 | 1.15E+00 | 6.79E−01 | 2 | Unchanged Medium | |
| P22223 | K19ef7 | cadherin 3: type 1; P-cadherin (p | 896.8467 | 1200 | 372.3054 | 823.6929 | −1.27E+00 | 4.22E−01 | 2 | Unchanged Medium | |
| P22676 | N07ab5 | calbindin 2: (29kD: calretinin) | 1781.134 | 1500 | 2116.438 | 1797.589 | 2.49E−01 | −2.52E−01 | 2 | Unchanged Medium | |
| Q99828 | O19gh1 | calcium and integrin binding 1 (c | 1130.551 | 1320 | 838.3709 | 1096.979 | −4.31E−01 | 2.26E−01 | 2 | Unchanged Medium | |
| P02593 | I18ab4 | calmodulin 1 (phosphorylase kina | 1076.299 | 787 | 983.6302 | 949.1389 | −1.30E−01 | 4.51E−01 | 2 | Unchanged Medium | |
| Q13542 | L01ab5 | calmodulin 3 (phosphorylase kina | 902.667 | 464 | 871.1455 | 746.0174 | −5.13E−02 | −9.59E−01 | 2 | Unchanged Medium | |
| P27824 | I05ef1 | calnexin | 1492.644 | 1370 | 1543.709 | 1470.4 | 4.85E−02 | −1.19E−01 | 2 | Unchanged Medium | |
| P17655 | C20ef6 | calpain 2: (m/II) large subunit | 671.5939 | 837 | 549.4365 | 685.847 | −2.90E−01 | 3.17E−01 | 2 | Unchanged Medium | |
| P04632 | C22ef6 | calpain: small subunit 1 | 1565.935 | 1650 | 1258.047 | 1490.643 | −3.16E−01 | 7.36E−02 | 2 | Unchanged Medium | |
| Q15417 | P17ab5 | calponin 3: acidic | 911.495 | 603 | 737.1204 | 750.6841 | −3.06E−01 | −5.95E−01 | 2 | Unchanged Medium | |
| Q9UEG5 | D17ef8 | capicua homolog (Drosophila) | 629.0213 | 486 | 439.6052 | 518.1163 | −5.17E−01 | −3.73E−01 | 2 | Unchanged Medium | |
| P52907 | O08ab4 | capping protein (actin filament) n | 848.0436 | 684 | 584.7035 | 705.5063 | −5.36E−01 | −3.11E−01 | 2 | Unchanged Medium | |
| Q9GZX3 | N16gh1 | carbohydrate (N-acetylglucosami | 793.1054 | 929 | 751.3352 | 824.5253 | −7.61E−02 | 2.28E−01 | 2 | Unchanged Medium | |
| Q16619 | K01ab4 | cardiotrophin 1 | 1478.249 | 1220 | 1559.477 | 1420.784 | 7.72E−02 | −2.72E−01 | 2 | Unchanged Medium | |
| Q9UBD9 | I24cd8 | cardiotrophin-like cytokine: neuro | 361.4077 | 893 | 489.6592 | 581.3157 | 4.38E−01 | 1.30E+00 | 2 | Unchanged Medium | |
| Q15699 | M16cd4 | cartilage paired-class homeoprot | 747.0317 | 596 | 1109.834 | 817.569 | 5.71E−01 | −3.26E−01 | 2 | Unchanged Medium | |
| P48729 | A22ab6 | casein kinase 1: alpha 1 | 690.8551 | 1150 | 902.9474 | 914.9218 | 3.86E−01 | 7.36E−01 | 2 | Unchanged Medium | |
| P48730 | L07ab4 | casein kinase 1: delta | 951.5238 | 1160 | 1111.571 | 1073.796 | 2.24E−01 | 2.84E−01 | 2 | Unchanged Medium | |
| P19138 | L08ef5 | casein kinase 2: alpha 1 polypep | 483.6062 | 597 | 461.8036 | 513.9702 | −6.66E−02 | 3.03E−01 | 2 | Unchanged Medium | |
| P49662 | C12ef6 | caspase 4; apoptosis-related cys | 2013.957 | 2460 | 1017.425 | 1831.485 | −9.85E−01 | 2.90E−01 | 2 | Unchanged Medium | |
| P21964 | F19ef6 | catechol-O-methyltransferase | 697.5886 | 1170 | 1268.869 | 1046.831 | 8.63E−01 | 7.51E−01 | 2 | Unchanged Medium | |
| P26232 | K01ef7 | catenin (cadherin-associated pro | 797.8639 | 717 | 810.257 | 774.9896 | 2.22E−02 | −1.54E−01 | 2 | Unchanged Medium | |
| P35222 | G07ef7 | catenin (cadherin-associated pro | 969.252 | 1290 | 1103.619 | 1121.277 | 1.87E−01 | 4.14E−01 | 2 | Unchanged Medium | |
| P07339 | N23ef7 | cathepsin D (lysosomal aspartyl | 1253.01 | 947 | 1133.879 | 1111.314 | −1.44E−01 | −4.04E−01 | 2 | Unchanged Medium | |
| Q99967 | C18cd6 | Cbp/p300-interacting transactiva | 1293.915 | 1600 | 2245.133 | 1711.366 | 7.95E−01 | 3.02E−01 | 2 | Unchanged Medium | |
| P49715 | N17ef6 | CCAAT/enhancer binding protein | 882.9323 | 884 | 1045.45 | 937.3612 | 2.44E−01 | 1.26E−03 | 2 | Unchanged Medium | |
| P53567 | N22ab5 | CCAAT/enhancer binding protein | 826.9569 | 1160 | 1584.869 | 1191.643 | 9.38E−01 | 4.92E−01 | 2 | Unchanged Medium | |
| O95627 | B21cd8 | CCR4-NOT transcription comple | 606.4629 | 851 | 692.4535 | 716.7046 | 1.91E−01 | 4.89E−01 | 2 | Unchanged Medium | |
| Q04900 | G02cd4 | CD164 antigen: sialomucin | 750.3086 | 570 | 673.7234 | 664.5986 | −1.55E−01 | −3.97E−01 | 2 | Unchanged Medium | |
| P07766 | P07ab3 | CD3E antigen: epsilon polypepti | 1808.209 | 1410 | 2643.721 | 1953.844 | 5.48E−01 | −3.59E−01 | 2 | Unchanged Medium | |
| P34810 | A21ab4 | CD68 antigen | 2460.227 | 1100 | 1293.961 | 1617.586 | −9.27E−01 | −1.16E+00 | 2 | Unchanged Medium | |
| P18582 | B21ab5 | CD81 antigen (target of antiprolif | 1303.144 | 938 | 1598.276 | 1279.761 | 2.95E−01 | −4.75E−01 | 2 | Unchanged Medium | |
| Q16181 | F09ef5 | CDC10 cell division cycle 10 hom | 426.6642 | 443 | 751.3623 | 540.3444 | 8.16E−01 | 5.42E−02 | 2 | Unchanged Medium | |
| Q16543 | H04ef5 | CDC37 cell division cycle 37 hom | 1085.897 | 1050 | 1336.155 | 1158.574 | 2.99E−01 | −4.35E−02 | 2 | Unchanged Medium | |
| O14735 | I14cd6 | CDP-diacylglycerol--inositol 3-ph | 241.7793 | 1180 | 311.7422 | 579.4931 | 3.67E−01 | 2.29E+00 | 2 | Unchanged Medium | |
| P30260 | F13ef5 | cell division cycle 27 | 1060.812 | 1470 | 1469.824 | 1334.733 | 4.70E−01 | 4.74E−01 | 2 | Unchanged Medium | |
| P49427 | B12ef7 | cell division cycle 34 | 762.5808 | 1300 | 766.6843 | 942.0784 | 7.74E−03 | 7.66E−01 | 2 | Unchanged Medium | |
| P29373 | F01ef7 | cellular retinoic acid binding prot | 1226.236 | 803 | 1350.177 | 1126.373 | 1.39E−01 | −6.11E−01 | 2 | Unchanged Medium | |
| P41208 | F09ab5 | centrin: EF-hand protein: 2 | 539.1633 | 672 | 676.8582 | 629.3345 | 3.28E−01 | 3.18E−01 | 2 | Unchanged Medium | |
| O00522 | F05ab4 | cerebral cavernous malformation | 870.4309 | 631 | 723.1482 | 741.4854 | −2.67E−01 | −4.64E−01 | 2 | Unchanged Medium | |
| O15183 | B12cd3 | CGG triplet repeat binding protei | 402.949 | 610 | 883.3283 | 632.0808 | 1.13E+00 | 5.98E−01 | 2 | Unchanged Medium | |
| Q9Y2Z5 | D07ef2 | CGI-06 protein | 721.6709 | 674 | 1177.641 | 857.6492 | 7.06E−01 | −9.94E−02 | 2 | Unchanged Medium | |
| Q9Y3C3 | J05ef2 | CGI-120 protein | 1229.537 | 1360 | 2124.304 | 1572.302 | 7.89E−01 | 1.49E−01 | 2 | Unchanged Medium | |
| Q9Y3D0 | J11ef2 | CGI-128 protein | 1592.787 | 2370 | 2011.685 | 1992.502 | 3.37E−01 | 5.75E−01 | 2 | Unchanged Medium | |
| Q9Y3E5 | J17ef2 | CGI-147 protein | 532.425 | 785 | 567.0634 | 629.3057 | 9.09E−02 | 5.66E−01 | 2 | Unchanged Medium | |
| Q9Y3E8 | C08ef2 | CGI-150 protein | 785.276 | 924 | 778.0449 | 829.1206 | −1.33E−02 | 2.35E−01 | 2 | Unchanged Medium | |
| Q9Y320 | A19ef2 | CGI-31 protein | 745.0314 | 932 | 703.7829 | 793.4541 | −8.22E−02 | 3.22E−01 | 2 | Unchanged Medium | |
| Q9Y357 | P16ef1 | CGI-40 protein | 817.7272 | 769 | 418.6337 | 668.441 | −9.66E−01 | −8.87E−02 | 2 | Unchanged Medium | |
| Q9Y360 | A10ef2 | CGI-45 protein | 786.0567 | 914 | 796.6229 | 832.2061 | 1.93E−02 | 2.17E−01 | 2 | Unchanged Medium | |
| Q9Y512 | B04ef3 | CGI-51 protein | 967.3508 | 859 | 1045.638 | 957.4201 | 1.12E−01 | −1.71E−01 | 2 | Unchanged Medium | |
| Q9Y390 | K21ef2 | CGI-81 protein | 1391.902 | 802 | 1140.01 | 1111.152 | −2.88E−01 | −7.96E−01 | 2 | Unchanged Medium | |
| P40227 | N20ab4 | chaperonin containing TCP1; sub | 1011.613 | 436 | 592.4944 | 679.9043 | −7.72E−01 | −1.22E+00 | 2 | Unchanged Medium | |
| Q99832 | H07cd6 | chaperonin containing TCP1; sub | 1190.644 | 1090 | 1548.874 | 1274.983 | 3.79E−01 | −1.33E−01 | 2 | Unchanged Medium | |
| Q9UBR5 | I23ef4 | chemokine-like factor 1 | 1047.63 | 1800 | 1059.437 | 1303.992 | 1.62E−02 | 7.85E−01 | 2 | Unchanged Medium | |
| Q9UHN8 | N05ef4 | chemokine-like factor 1 | 1568.851 | 2030 | 1237.728 | 1611.343 | −3.42E−01 | 3.70E−01 | 2 | Unchanged Medium | |
| P54105 | L07ef5 | chloride channel; nucleotide-sen | 471.0775 | 487 | 640.6655 | 532.9217 | 4.44E−01 | 4.80E−02 | 2 | Unchanged Medium | |
| Q14781 | F09ef1 | chromobox homolog 2 (Pc class | 795.873 | 749 | 713.815 | 753.0305 | −1.57E−01 | −8.68E−02 | 2 | Unchanged Medium | |
| Q9BXS4 | K12cd5 | chromosome 1 open reading fra | 874.0189 | 1370 | 904.3066 | 1050.603 | 4.91E−02 | 6.52E−01 | 2 | Unchanged Medium | |
| Q9NPA0 | H07gh4 | chromosome 11 hypothetical pro | 998.8577 | 834 | 858.794 | 897.3389 | −2.16E−01 | −2.60E−01 | 2 | Unchanged Medium | |
| Q9UKR5 | G14cd7 | chromosome 14 open reading fra | 1181.962 | 1160 | 1285.11 | 1207.747 | 1.21E−01 | −3.18E−02 | 2 | Unchanged Medium | |
| P56378 | M16cd5 | chromosome 14 open reading fra | 503.5233 | 690 | 890.9975 | 694.9486 | 8.23E−01 | 4.55E−01 | 2 | Unchanged Medium | |
| O95433 | B22cd7 | chromosome 14 open reading fra | 656.1775 | 563 | 438.9755 | 552.8428 | −5.80E−01 | −2.20E−01 | 2 | Unchanged Medium | |
| Q9NV31 | J23gh3 | chromosome 15 open reading fra | 853.8367 | 832 | 861.2221 | 848.9729 | 1.24E−02 | −3.76E−02 | 2 | Unchanged Medium | |
| Q9NVE2 | D09gh3 | chromosome 2 open reading fra | 648.49 | 671 | 468.6352 | 596.0999 | 4.69E−01 | 4.96E−02 | 2 | Unchanged Medium | |
| Q9UI05 | L04gh4 | chromosome 20 open reading fra | 2518.951 | 2140 | 1201.051 | 1952.881 | −1.07E+00 | −2.36E−01 | 2 | Unchanged Medium | |
| Q9P0A7 | M09ef8 | chromosome 20 open reading fra | 581.9098 | 1080 | 1200.665 | 954.524 | 1.04E+00 | 8.93E−01 | 2 | Unchanged Medium | |
| Q9Y3B1 | H22ef1 | chromosome 20 open reading fra | 1691.864 | 1230 | 783.7799 | 1235.108 | −1.11E+00 | −4.60E−01 | 2 | Unchanged Medium | |
| Q9BQ89 | C01gh8 | chromosome 20 open reading fra | 1460.875 | 952 | 1221.732 | 1211.641 | −2.58E−01 | −6.17E−01 | 2 | Unchanged Medium | |
| Q96C58 | J19gh7 | chromosome 6 open reading fra | 1254.647 | 1080 | 480.9999 | 940.0036 | −1.38E+00 | −2.10E−01 | 2 | Unchanged Medium | |
| Q9Y5Z4 | K12ef3 | chromosome 6 open reading fra | 915.3818 | 988 | 372.3119 | 758.6366 | −1.30E+00 | 1.10E−01 | 2 | Unchanged Medium | |
| Q00610 | K12cd5 | clathrin; heavy polypeptide (Hc) | 1639.759 | 1210 | 1437.44 | 1427.503 | −1.90E−01 | −4.44E−01 | 2 | Unchanged Medium | |
| P56749 | F04cd7 | claudin 12 | 2262.278 | 2480 | 1233.472 | 1992.755 | −8.75E−01 | 1.34E−01 | 2 | Unchanged Medium | |
| O43809 | M15cd7 | cleavage and polyadenylation sp | 640.2301 | 503 | 683.3467 | 608.9825 | 9.40E−02 | −3.47E−01 | 2 | Unchanged Medium | |
| O96005 | G05ab4 | cleft lip and palate associated tra | 1353.338 | 1170 | 1394.845 | 1304.597 | 4.36E−02 | −2.15E−01 | 2 | Unchanged Medium | |
| Q16740 | E20cd4 | ClpP caseinolytic protease: ATP- | 625.8711 | 528 | 475.0848 | 543.0367 | −3.98E−01 | −2.45E−01 | 2 | Unchanged Medium | |
| P55085 | O24ab5 | coagulation factor II (thrombin) re | 829.7537 | 979 | 392.7495 | 734 | −1.08E+00 | 2.39E−01 | 2 | Unchanged Medium | |
| P13726 | B05ab4 | coagulation factor III (thrombopla | 1079.841 | 880 | 442.6212 | 800.9723 | −1.29E+00 | −2.94E−01 | 2 | Unchanged Medium | |
| Q9Y678 | D14ef8 | coat protein gamma-cop | 815.7993 | 738 | 1009.756 | 854.5161 | 3.08E−01 | −1.45E−01 | 2 | Unchanged Medium | |
| Q15363 | M11ef3 | coated vesicle membrane protein | 706.3054 | 804 | 870.2118 | 793.6589 | 3.01E−01 | 1.88E−01 | 2 | Unchanged Medium | |
| O43513 | N06cd4 | cofactor required for Sp1 transcri | 591.8619 | 481 | 798.8706 | 623.9284 | 4.33E−01 | −2.99E−01 | 2 | Unchanged Medium | |
| Q9Y281 | N18cd8 | cofilin 2 (muscle | 671.9394 | 270 | 1093.534 | 678.6369 | 7.03E−01 | −1.31E+00 | 2 | Unchanged Medium | |
| P04141 | G22gh6 | colony stimulating factor 2 (gran | 116.2139 | 1290 | 165.718 | 522.783 | 5.12E−01 | 3.47E+00 | 2 | Unchanged Medium | |
| P02745 | L06ef1 | complement component 1; q sub | 683.4431 | 1650 | 798.2366 | 1042.465 | 2.24E−01 | 1.27E+00 | 2 | Unchanged Medium | |
| P29279 | I16ab4 | connective tissue growth factor | 596.4123 | 537 | 1071.517 | 734.8489 | 8.45E−01 | −1.52E−01 | 2 | Unchanged Medium | |
| O43191 | B02cd3 | COP9 constitutive photomorphog | 655.8616 | 1030 | 1302.334 | 1062.925 | 6.06E−01 | 2.68E−01 | 2 | Unchanged Medium | |
| Q99627 | C01cd7 | COP9 homolog | 788.2297 | 968 | 1195.497 | 984.0634 | 6.01E−01 | 2.97E−01 | 2 | Unchanged Medium | |
| O15387 | G21cd7 | COP9 subunit 6 (MOV34 homolog | 590.9973 | 382 | 628.5041 | 533.8251 | 8.88E−02 | −6.30E−01 | 2 | Unchanged Medium | |
| Q9ULV4 | K18ef3 | coronin; actin binding protein: IC | 1594.037 | 1200 | 1362.545 | 1385.839 | −2.26E−01 | −4.09E−01 | 2 | Unchanged Medium | |
| Q9BR76 | I14gh7 | coronin; actin-binding protein; 1E | 708.5853 | 1080 | 690.8568 | 827.7864 | −3.65E−02 | 6.13E−01 | 2 | Unchanged Medium | |
| Q14061 | B22cd5 | COX17 homolog; cytochrome c | 889.1581 | 1440 | 745.6658 | 1026.374 | −2.54E−01 | 7.00E−01 | 2 | Unchanged Medium | |
| Q9Y6B2 | E02ef8 | CREBBP/EP300 inhibitory protei | 946.9513 | 1030 | 1988.725 | 1323.308 | 1.07E+00 | 1.27E−01 | 2 | Unchanged Medium | |
| Q9NY68 | C06gh4 | CTL2 gene | 1381.821 | 804 | 788.5002 | 991.4995 | −8.09E−01 | −7.81E−01 | 2 | Unchanged Medium | |
| P24385 | O04ef5 | cyclin D1 (PRAD1: parathyroid a | 1214.611 | 2150 | 1100.478 | 1486.769 | −1.42E−01 | 8.21E−01 | 2 | Unchanged Medium | |
| Q9NXT4 | K09gh2 | cyclin M2 | 1560.659 | 1720 | 1582.768 | 1620.99 | 2.03E−02 | 1.40E−01 | 2 | Unchanged Medium | |
| P01034 | I11ab6 | cystatin C (amyloid angiopathy a | 1067.127 | 970 | 1366.396 | 1134.612 | 3.57E−01 | −1.37E−01 | 2 | Unchanged Medium | |
| Q15828 | I13ab4 | cystatin E/M | 967.1669 | 2790 | 451.3932 | 1404.267 | −1.10E+00 | 1.53E+00 | 2 | Unchanged Medium | |
| P01036 | C02ab6 | cystatin S | 1261.605 | 466 | 1069.756 | 932.4892 | −2.38E−01 | −1.44E+00 | 2 | Unchanged Medium | |
| P52943 | I05ab4 | cysteine-rich protein 2 | 874.1939 | 499 | 1277.819 | 883.82 | 5.48E−01 | −8.08E−01 | 2 | Unchanged Medium | |
| P13498 | G18ab3 | cytochrome b-245; alpha polypep | 708.9954 | 1050 | 359.3156 | 707.7483 | −9.81E−01 | 5.73E−01 | 2 | Unchanged Medium | |
| P00167 | C04ab6 | cytochrome b-5 | 2187.128 | 1480 | 1034.311 | 1565.893 | −1.08E+00 | −5.67E−01 | 2 | Unchanged Medium | |
| P00001 | N24gh6 | cytochrome c | 547.6708 | 442 | 517.6125 | 502.4786 | −8.14E−02 | −3.09E−01 | 2 | Unchanged Medium | |
| P10606 | I24gh6 | cytochrome c oxidase subunit Vb | 1364.01 | 2100 | 2087.456 | 1851.225 | 6.14E−01 | 6.24E−01 | 2 | Unchanged Medium | |
| P08574 | P10ab5 | cytochrome c-1 | 667.9346 | 443 | 441.6771 | 517.4383 | −5.97E−01 | −5.93E−01 | 2 | Unchanged Medium | |
| Q99426 | C17gh1 | cytosketeton-associated protein | 560.9411 | 684 | 777.3269 | 673.9633 | 4.71E−01 | 2.85E−01 | 2 | Unchanged Medium | |
| Q07065 | G13cd7 | cytoskeleton-associated protein | 602.5101 | 575 | 1785.415 | 987.5887 | 1.57E+00 | −6.78E−02 | 2 | Unchanged Medium | |
| Q15038 | L13ef3 | DAZ associated protein 2 | 808.0755 | 890 | 564.7611 | 754.4305 | −5.17E−01 | 1.40E−01 | 2 | Unchanged Medium | |
| Q9H2L4 | B18gh8 | DC32 | 408.0427 | 702 | 780.2878 | 630.0414 | 9.35E−01 | 7.82E−01 | 2 | Unchanged Medium | |
| Q9NPA8 | G22gh7 | DC6 protein | 944.3094 | 1570 | 1514.164 | 1342.189 | 6.81E−01 | 7.32E−01 | 2 | Unchanged Medium | |
| O60231 | H13cd3 | DEAD/H (Asp-Glu-Ala-Asp/His) | 753.8815 | 873 | 799.6561 | 808.7915 | 8.50E−02 | 2.11E−01 | 2 | Unchanged Medium | |
| Q9GZR7 | E02gh4 | DEAD/H (Asp-Glu-Ala-Asp/His) | 803.42 | 984 | 774.3765 | 554.0965 | −5.31E−02 | 2.93E−01 | 2 | Unchanged Medium | |
| P17844 | F04ab4 | DEAD/H (Asp-Glu-Ala-Asp/His) | 1482.642 | 1440 | 1670.104 | 1530.749 | 1.72E−01 | −4.26E−02 | 2 | Unchanged Medium | |
| Q08211 | M03ab4 | DEAD/H (Asp-Glu-Ala-Asp/His) | 907.5859 | 658 | 907.4343 | 824.2193 | −2.41E−04 | −4.65E−01 | 2 | Unchanged Medium | |
| Q9UJV9 | D08ef2 | DEAD-box protein abstrakt | 976.8378 | 1230 | 870.3672 | 1024.437 | −1.66E−01 | 3.28E−01 | 2 | Unchanged Medium | |
| P51398 | G18ef6 | death associated protein 3 | 920.3044 | 981 | 814.3518 | 905.1904 | −1.76E−01 | 9.20E−02 | 2 | Unchanged Medium | |
| Q13437 | G22cd4 | Deleted in split-hand/split-foot 1 | 1565.344 | 1500 | 1721.872 | 1596.672 | 1.37E−01 | −5.88E−02 | 2 | Unchanged Medium | |
| O60735 | O06cd6 | dendritic cell protein | 858.6375 | 502 | 467.3432 | 609.2393 | −8.78E−01 | −7.75E−01 | 2 | Unchanged Medium | |
| Q16854 | M06ab5 | deoxyguanosine kinase | 858.1918 | 885 | 989.3036 | 910.8082 | 2.05E−01 | 4.43E−02 | 2 | Unchanged Medium | |
| Q9Y295 | H04ab8 | developmentally regulated GTP | 736.3786 | 634 | 660.7023 | 676.9267 | −1.56E−01 | −2.17E−01 | 2 | Unchanged Medium | |
| P00387 | F09ab3 | diaphorase (NADH) (cytochrome | 1402.134 | 1030 | 1435.74 | 1290.593 | 3.42E−02 | 4.40E−01 | 2 | Unchanged Medium | |
| Q9UHY9 | I08ef2 | dicarbonyl/L-xylulose reductase | 544.2125 | 740 | 938.7807 | 741.1 | 7.87E−01 | 4.44E−01 | 2 | Unchanged Medium | |
| P36957 | C06ab6 | dihydrolipoamide S-succinyltrans | 625.9037 | 713 | 393.7165 | 577.6196 | −6.69E−01 | 1.88E−01 | 2 | Unchanged Medium | |
| Q99075 | M12ef7 | diphtheria toxin receptor(heparin | 721.8431 | 638 | 197.117 | 518.9525 | −1.87E+00 | −1.78E−01 | 2 | Unchanged Medium | |
| Q9UFN1 | H10gh6 | DKFZP564B147 protein | 966.5927 | 947 | 1469.105 | 1127.658 | 6.04E−01 | −2.91E−02 | 2 | Unchanged Medium | |
| O95882 | C13ef8 | DKFZP564C1940 protein | 941.4076 | 865 | 804.639 | 870.3185 | −2.26E−01 | −1.22E−01 | 2 | Unchanged Medium | |
| Q9Y269 | C07ef8 | DKFZP564M082 protein | 675.8991 | 1110 | 1208.543 | 998.4411 | 8.38E−01 | 7.17E−01 | 2 | Unchanged Medium | |
| Q9NW63 | J11ef8 | DKFZP566C243 protein | 419.5607 | 770 | 625.8483 | 605.084 | 5.77E−01 | 8.76E−01 | 2 | Unchanged Medium | |
| Q9H6Y7 | H04gh6 | DKFZP566H073 protein | 1390.389 | 1480 | 1191.195 | 1352.292 | −2.23E−01 | 8.55E−02 | 2 | Unchanged Medium | |
| Q9Y3X0 | P23ef8 | DKFZP586M1019 protein | 588.3603 | 505 | 672.4872 | 588.5767 | 1.93E−01 | −2.21E−01 | 2 | Unchanged Medium | |
| O60762 | G19cd4 | dolichyl-phosphate mannosyltran | 407.0059 | 581 | 605.8353 | 531.3373 | 5.74E−01 | 5.14E−01 | 2 | Unchanged Medium | |
| P53805 | B11ab5 | Down syndrome critical region g | 370.0907 | 663 | 511.9248 | 515.0794 | 4.68E−01 | 8.42E−01 | 2 | Unchanged Medium | |
| Q13597 | N13ef3 | downregulated in ovarian cancer | 594.6931 | 693 | 623.702 | 636.966 | 6.87E−02 | 2.20E−01 | 2 | Unchanged Medium | |
| Q9C005 | J05gh8 | dpy-30-like protein | 810.4585 | 1210 | 1174.001 | 1064.132 | 5.35E−01 | 5.76E−01 | 2 | Unchanged Medium | |
| Q13448 | J03cd6 | DR1-associated protein 1 (negati | 1240.359 | 1140 | 2382.895 | 1586.247 | 9.42E−01 | −1.27E−01 | 2 | Unchanged Medium | |
| Q9UHF9 | K01ef4 | dual oxidase 2 | 1146.14 | 648 | 316.6462 | 703.5318 | −1.86E+00 | −8.23E−01 | 2 | Unchanged Medium | |
| P51452 | N22ab4 | dual specificity phosphatase 3 (v | 649.6813 | 605 | 473.5652 | 576.1845 | −4.56E−01 | −1.02E−01 | 2 | Unchanged Medium | |
| Q14203 | A13ab6 | dynactin 1 (p150; glued homolog | 507.5942 | 529 | 623.6948 | 553.5576 | 2.97E−01 | 6.06E−02 | 2 | Unchanged Medium | |
| Q13561 | D19cd6 | dynactin 2 (p50) | 1565.567 | 1380 | 1173.024 | 1372.281 | −4.16E−01 | −1.84E−01 | 2 | Unchanged Medium | |
| Q13409 | I16ef1 | dynein: cytoplasmic: intermediate | 793.3207 | 604 | 464.9675 | 620.6506 | −7.71E−01 | −3.94E−01 | 2 | Unchanged Medium | |
| Q09472 | C18ab4 | E1A binding protein p300 | 1496.409 | 2120 | 1342.944 | 1651.644 | −1.56E−01 | 5.00E−01 | 2 | Unchanged Medium | |
| | D20gh8 | EAF1 protein | 642.1587 | 652 | 469.6834 | 588.0816 | −4.51E−01 | 2.28E−02 | 2 | Unchanged Medium | |
| P78365 | N19ab4 | early development regulator 2 (p | 894.3715 | 1070 | 761.5923 | 907.5945 | −2.32E−01 | 2.54E−01 | 2 | Unchanged Medium | |
| Q99848 | G11cd1 | EBNA1 binding protein 2 | 977.8671 | 611 | 818.6583 | 802.3778 | −2.56E−01 | −6.79E−01 | 2 | Unchanged Medium | |
| Q12805 | A21ab6 | EGF-containing fibulin-like extra | 1002.82 | 332 | 458.9563 | 597.7763 | −1.13E+00 | −1.60E+00 | 2 | Unchanged Medium | |
| Q9Y6I3 | D21cd8 | EH domain-binding mitotic phosp | 736.8984 | 500 | 629.1845 | 621.9752 | −2.28E−01 | −5.60E−01 | 2 | Unchanged Medium | |
| Q15717 | C04ab4 | ELAV (embryonic lethal; abnorm | 850.8493 | 800 | 843.4463 | 831.3485 | −1.26E−02 | −8.94E−02 | 2 | Unchanged Medium | |
| P13804 | I24ab3 | electron-transfer-flavoprotein: alp | 918.8391 | 545 | 529.2866 | 664.3533 | −7.96E−01 | −7.54E−01 | 2 | Unchanged Medium | |
| Q14247 | K17ef6 | ems 1 sequence (mammary tumo | 1135.188 | 1050 | 1002.084 | 1063.9 | −1.80E−01 | −1.06E−01 | 2 | Unchanged Medium | |
| P42892 | O21ab4 | endothelin converting enzyme 1 | 602.4294 | 799 | 809.7071 | 737.1982 | 4.27E−01 | 4.08E−01 | 2 | Unchanged Medium | |
| P30084 | A17ab6 | enoyl Coenzyme A hydratase; sh | 802.3114 | 1210 | 741.2226 | 917.9548 | −1.14E−01 | 5.93E−01 | 2 | Unchanged Medium | |
| P07099 | I20ab3 | epoxide hydrolase 1; microsomal | 1231.48 | 1840 | 1198.169 | 1424.661 | −3.96E−02 | 5.83E−01 | 2 | Unchanged Medium | |
| P29692 | H04ab5 | eukaryotic translation elongation | 1329.756 | 744 | 1110.87 | 1061.631 | −2.59E−01 | −8.37E−01 | 2 | Unchanged Medium | |
| P20042 | L05cd4 | eukaryotic translation initiation fa | 1489.433 | 603 | 956.276 | 1016.24 | −6.39E−01 | −1.30E+00 | 2 | Unchanged Medium | |
| P41091 | A20ab4 | eukaryotic translation initiation fa | 742.5843 | 474 | 478.7249 | 565.2535 | −6.33E−01 | −6.46E−01 | 2 | Unchanged Medium | |
| Q99613 | L16cd3 | eukaryotic translation initiation fa | 1140.022 | 853 | 998.7488 | 997.2011 | −1.91E−01 | −4.19E−01 | 2 | Unchanged Medium | |
| P78344 | C02ab4 | eukaryotic translation initiation fa | 1562.952 | 1520 | 1177.845 | 1419.665 | −4.08E−01 | −4.19E−02 | 2 | Unchanged Medium | |
| P23588 | A24ab4 | eukaryotic translation initiation fa | 1343.019 | 1010 | 1125.825 | 1160.659 | −2.54E−01 | −4.07E−01 | 2 | Unchanged Medium | |
| Q13541 | C03ab5 | eukaryotic translation initiation fa | 1655.926 | 1060 | 2293.236 | 1669.873 | 4.70E−01 | −6.43E−01 | 2 | Unchanged Medium | |
| Q13542 | A19ab6 | eukaryotic translation initiation fa | 1620.111 | 1850 | 1663.197 | 1712.605 | 3.79E−02 | 1.95E−01 | 2 | Unchanged Medium | |
| O60573 | G16cd5 | eukaryotic translation initiation fa | 799.7789 | 883 | 819.3679 | 833.9147 | 3.49E−02 | 1.42E−01 | 2 | Unchanged Medium | |
| Q16394 | K02ab3 | exostoses (multiple) 1 | 513.7526 | 428 | 813.2693 | 584.9471 | 6.63E−01 | −2.64E−01 | 2 | Unchanged Medium | |
| P37268 | C17ab6 | farnesyl-diphosphate farnesyltra | 726.0862 | 455 | 649.0518 | 610.2023 | −1.62E−01 | −6.73E−01 | 2 | Unchanged Medium | |
| P55899 | L22ab5 | Fc fragment of IgG; receptor: tra | 450.8629 | 771 | 476.1039 | 566.0193 | 7.86E−02 | 7.74E−01 | 2 | Unchanged Medium | |
| Q9UK73 | P05ef3 | fem-1 homolog b (C. elegans) | 548.2677 | 547 | 530.801 | 541.9489 | −4.67E−02 | −3.93E−03 | 2 | Unchanged Medium | |
| P21333 | G14ab4 | filamin A; alpha (actin binding pr | 946.0465 | 675 | 1277.458 | 966.2982 | 4.33E−01 | −4.86E−01 | 2 | Unchanged Medium | |
| P26885 | P07ab4 | FK506 binding protein 2 (13kD) | 814.6357 | 510 | 661.4593 | 662.0387 | −3.01E−01 | −6.76E−01 | 2 | Unchanged Medium | |
| Q13451 | F15ab4 | FK506 binding protein 5 | 1366.905 | 1110 | 812.8568 | 1097.248 | −7.50E−01 | −2.98E−01 | 2 | Unchanged Medium | |
| O95633 | M01cd6 | follistatin-like 3 (secreted glycop | 1312.174 | 1280 | 1092.99 | 1227.557 | −2.64E−01 | −3.86E−02 | 2 | Unchanged Medium | |
| P15407 | A20cd4 | FOS-like antigen 1 | 1696.135 | 1350 | 1218.491 | 1421.01 | −4.77E−01 | −3.31E−01 | 2 | Unchanged Medium | |
| Q14192 | G02ab4 | four and a half LIM domains 2 | 309.9219 | 591 | 1210.635 | 703.7424 | 1.97E+00 | 9.30E−01 | 2 | Unchanged Medium | |
| Q9NZA0 | N17gh6 | fuse-binding protein-interacting r | 924.2347 | 911 | 1110.468 | 981.791 | 2.65E−01 | −2.13E−02 | 2 | Unchanged Medium | |
| P35637 | P19ab4 | fusion: derived from (12;16) mali | 866.3996 | 654 | 1159.792 | 893.3381 | 4.21E−01 | −4.06E−01 | 2 | Unchanged Medium | |
| | E21gh7 | FXYD domain-containing ion tra | 942.6126 | 1060 | 802.8679 | 935.6666 | −2.32E−01 | 1.71E−01 | 2 | Unchanged Medium | |
| Q9Y653 | B02cd5 | G protein-coupled receptor 56 | 2010.489 | 2160 | 1501.617 | 1889.081 | −4.21E−01 | 1.00E−01 | 2 | Unchanged Medium | |
| Q9H1C0 | E18gh4 | G protein-coupled receptor 92 | 712.1007 | 750 | 719.7999 | 727.2869 | 1.55E−02 | 7.47E−02 | 2 | Unchanged Medium | |
| P15170 | L13ab4 | G1 to S phase transition 1 | 2042.425 | 1410 | 1109.721 | 1521.226 | −8.80E−01 | −5.33E−01 | 2 | Unchanged Medium | |
| O95166 | D09cd7 | GABA(A) receptor-associated pr | 1280.495 | 2210 | 1227.378 | 1572.36 | −6.11E−02 | 7.87E−01 | 2 | Unchanged Medium | |
| O08765 | D21cd7 | GABA(A) receptor-associated pr | 1241.27 | 1130 | 918.8337 | 1095.839 | −4.34E−01 | −1.39E−01 | 2 | Unchanged Medium | |
| P16278 | O05ef1 | galactosidase; beta 1 | 539.4222 | 800 | 656.0518 | 665.3025 | 2.82E−01 | 5.69E−01 | 2 | Unchanged Medium | |
| P78537 | J23ab4 | GCN5 general control of amino-a | 813.3309 | 637 | 919.5196 | 790.0409 | 1.77E−01 | −3.52E−01 | 2 | Unchanged Medium | |
| P31150 | F14ab4 | GDP dissociation inhibitor 1 | 633.6157 | 894 | 640.108 | 722.7008 | 1.47E−02 | 4.97E−01 | 2 | Unchanged Medium | |
| P06396 | B23ab3 | gelsolin (amyloidosis: Finnish typ | 330.1567 | 577 | 633.2269 | 513.4286 | 9.40E−01 | 8.05E−01 | 2 | Unchanged Medium | |
| P52657 | E03ab6 | general transcription factor IIA; 2 | 538.6557 | 685 | 700.1857 | 641.1721 | 3.78E−01 | 3.46E−01 | 2 | Unchanged Medium | |
| Q00403 | F08ef6 | general transcription factor IIB | 500.4565 | 791 | 454.8328 | 582.2035 | −1.38E−01 | 6.61E−01 | 2 | Unchanged Medium | |
| P48060 | I23cd7 | glioma pathogenesis-related prol | 676.4844 | 692 | 752.7928 | 707.2232 | 1.54E−01 | 3.35E−02 | 2 | Unchanged Medium | |
| P06744 | B19ab3 | glucose phosphate isomerase | 900.9772 | 553 | 726.4749 | 726.7688 | −3.11E−01 | −7.05E−01 | 2 | Unchanged Medium | |
| P30101 | G13ab5 | glucose regulated protein: 58kD | 1708.658 | 1250 | 2220.105 | 1726.668 | 3.78E−01 | −4.50E−01 | 2 | Unchanged Medium | |
| O43836 | I22ef1 | glutamate receptor: ionotropic; N | 1729.777 | 1890 | 1655.131 | 1758.853 | −6.36E−02 | 1.29E−01 | 2 | Unchanged Medium | |
| P15104 | H22ab5 | glutamate-ammonia ligase (gluta | 1170.59 | 1040 | 1180.088 | 1130.151 | 1.17E−02 | −1.71E−01 | 2 | Unchanged Medium | |
| Q06210 | A23ab5 | glutamine-fructose-6-phosphate | 869.494 | 600 | 863.157 | 777.6585 | −1.06E−02 | −5.34E−01 | 2 | Unchanged Medium | |
| Q9Y3D4 | M19ef2 | glutaredoxin 2 | 888.5406 | 894 | 1099.209 | 960.4756 | 3.07E−01 | 6.32E−03 | 2 | Unchanged Medium | |
| Q9Y2Q3 | C24ef2 | glutathione S-transferase subuni | 841.3959 | 346 | 414.7892 | 533.8957 | −1.02E+00 | −1.28E+00 | 2 | Unchanged Medium | |
| P48637 | D01ab3 | glutathione synthetase | 650.8496 | 429 | 488.6663 | 522.9685 | −4.13E−01 | −6.00E−01 | 2 | Unchanged Medium | |
| P78417 | E20cd5 | glutathione-S-transferase like: gl | 1983.626 | 2020 | 1805.489 | 1937.888 | −1.36E−01 | 2.95E−02 | 2 | Unchanged Medium | |
| P49840 | J14ef4 | glycogen synthase kinase 3 alph | 1057.368 | 1460 | 1247.816 | 1254.087 | 2.39E−01 | 4.63E−01 | 2 | Unchanged Medium | |
| P13224 | F23ab3 | glycoprotein lb (platelet): beta p | 487.4684 | 530 | 653.4727 | 557.0977 | 4.23E−01 | 1.22E−01 | 2 | Unchanged Medium | |
| Q9UBQ7 | L08cd7 | glyoxylate reductase/hydroxypyr | 947.0262 | 977 | 997.593 | 973.9926 | 7.50E−02 | 4.55E−02 | 2 | Unchanged Medium | |
| Q9P2T1 | C02ef2 | GMPR2 for guanosine monophos | 762.0126 | 1070 | 817.0496 | 882.4154 | 1.01E−01 | 4.87E−01 | 2 | Unchanged Medium | |
| Q9H4A6 | I04gh5 | golgi phosphoprotein 3 (coat-pro | 1027.603 | 1210 | 1209.783 | 1148.94 | 2.35E−01 | 2.35E−01 | 2 | Unchanged Medium | |
| P28799 | B19ab5 | granulin | 1611.298 | 1750 | 1238.745 | 1532.188 | −3.79E−01 | 1.16E−01 | 2 | Unchanged Medium | |
| Q14393 | H10ab3 | growth arrest-specific 6 | 484.489 | 563 | 739.1661 | 595.5619 | 6.09E−01 | 2.17E−01 | 2 | Unchanged Medium | |
| P29354 | H06ef5 | growth factor receptor-bound pro | 721.5894 | 854 | 663.0587 | 746.3732 | −1.22E−01 | 2.44E−01 | 2 | Unchanged Medium | |
| Q14451 | D20ab4 | growth factor receptor-bound pro | 712.3296 | 846 | 636.8962 | 731.6029 | −1.61E−01 | 2.47E−01 | 2 | Unchanged Medium | |
| Q9Y6G2 | EO4ef8 | growth hormone inducible transm | 1289.256 | 1020 | 974.263 | 1093.42 | −4.04E−01 | −3.43E−01 | 2 | Unchanged Medium | |
| P04899 | J09ab4 | guanine nucleotide binding prote | 860.9208 | 1390 | 1748.649 | 1334.01 | 1.02E+00 | 6.94E−01 | 2 | Unchanged Medium | |
| P11016 | M13ef5 | guanine nucleotide binding prote | 758.629 | 773 | 836.6295 | 789.4586 | 1.41E−01 | 2.73E−02 | 2 | Unchanged Medium | |
| Q13905 | G09ab6 | guanine nucleotide-releasing fac | 410.0949 | 569 | 535.1889 | 504.61 | 3.84E−01 | 4.71E−01 | 2 | Unchanged Medium | |
| Q16774 | L16ab3 | guanylate kinase 1 | 968.5867 | 1340 | 975.8911 | 1095.967 | 1.08E−02 | 4.72E−01 | 2 | Unchanged Medium | |
| Q9Y649 | K16ef4 | GW128 protein | 1663.305 | 2420 | 1834.174 | 1971.464 | 1.41E−01 | 5.39E−01 | 2 | Unchanged Medium | |
| P07305 | G13ab6 | H1 histone family: member 0 | 1780.353 | 1870 | 1746.555 | 1797.448 | −2.77E−02 | 6.73E−02 | 2 | Unchanged Medium | |
| O75367 | M14cd5 | H2A histone family: member Y | 1065.311 | 837 | 455.5967 | 786.0181 | −1.23E+00 | −3.46E−01 | 2 | Unchanged Medium | |
| P17317 | C24ab6 | H2A histone famity: member Z | 591.0013 | 461 | 1570.489 | 874.2269 | 1.41E+00 | −3.58E−01 | 2 | Unchanged Medium | |
| P02278 | M16gh6 | H2B histone family: member G | 586.8773 | 852 | 684.6287 | 707.973 | 2.22E−01 | 5.38E−01 | 2 | Unchanged Medium | |
| Q9NS37 | A03gh5 | HCF-binding transcription factor | 857.3143 | 717 | 601.7268 | 725.1835 | −5.11E−01 | −2.59E−01 | 2 | Unchanged Medium | |
| O96004 | E02cd5 | heart and neural crest derivatives | 705.5163 | 612 | 621.033 | 646.3401 | −1.84E−01 | −2.04E−01 | 2 | Unchanged Medium | |
| P10809 | M11ef5 | heat shock 60kD protein 1 (chap | 1693.23 | 1020 | 1276.41 | 1330.235 | −4.08E−01 | 7.30E−01 | 2 | Unchanged Medium | |
| P34932 | I11ef1 | heat shock 70kD protein 4 | 1402.505 | 1610 | 1042.113 | 1352.283 | −4.28E−01 | 2.01E−01 | 2 | Unchanged Medium | |
| P07900 | G18ef7 | heat shock 90kD protein 1; alpha | 2405.793 | 1430 | 1512.258 | 1783.374 | −6.70E−01 | −7.48E−01 | 2 | Unchanged Medium | |
| P08238 | B20ab7 | heat shock 90kD protein 1; beta | 2781.462 | 1950 | 979.1145 | 1903.993 | −1.51E+00 | −5.11E−01 | 2 | Unchanged Medium | |
| O75506 | H17ab6 | heat shock factor binding protein | 1132.126 | 1300 | 1081.748 | 1171.256 | −6.57E−02 | 1.99E−01 | 2 | Unchanged Medium | |
| Q9UHG4 | K18ef4 | heme-regulated initiation factor 2 | 413.2841 | 659 | 576.3971 | 549.5035 | 4.80E−01 | 6.73E−01 | 2 | Unchanged Medium | |
| P09105 | J21ab4 | hemoglobin: theta 1 | 702.445 | 484 | 769.2336 | 651.9692 | 1.31E−01 | −5.37E−01 | 2 | Unchanged Medium | |
| O43504 | D23cd6 | hepatitis B virus x interacting pro | 1008.655 | 1340 | 949.0116 | 1097.672 | −8.79E−02 | 4.05E−01 | 2 | Unchanged Medium | |
| Q9H2I6 | N10gh5 | hepatitis C virus core-binding pro | 676.7297 | 620 | 473.9328 | 590.1783 | −5.14E−01 | −1.27E−01 | 2 | Unchanged Medium | |
| P51858 | K16ef7 | hepatoma-derived growth factor | 894.1008 | 1170 | 490.7456 | 850.7213 | −8.65E−01 | 3.85E−01 | 2 | Unchanged Medium | |
| Q04150 | A08ab7 | heterogeneous nuclear ribonucle | 943.3857 | 870 | 1065.597 | 959.787 | 1.76E−01 | −1.16E−01 | 2 | Unchanged Medium | |
| Q13151 | E15cd7 | heterogeneous nuclear ribonucle | 906.986 | 944 | 1143.552 | 998.1703 | 3.34E−01 | 5.77E−02 | 2 | Unchanged Medium | |
| P22626 | N09ab6 | heterogeneous nuclear ribonucle | 1021.87 | 1010 | 1584.855 | 1206.298 | 6.33E−01 | −1.38E−02 | 2 | Unchanged Medium | |
| P07910 | A10ab7 | heterogeneous nuclear ribonucle | 747.5309 | 718 | 1059.349 | 841.6601 | 5.03E−01 | −5.79E−02 | 2 | Unchanged Medium | |
| O14979 | L09cd5 | heterogeneous nuclear ribonucle | 1057.699 | 1990 | 1646.172 | 1563.717 | 6.38E−01 | 9.10E−01 | 2 | Unchanged Medium | |
| P52597 | G06ab7 | heterogeneous nuclear ribonucle | 1902.939 | 1280 | 1517.274 | 1567.536 | −3.27E−01 | −5.69E−01 | 2 | Unchanged Medium | |
| Q07244 | N13ab6 | heterogeneous nuclear ribonucle | 847.6967 | 667 | 684.4978 | 733.1055 | −3.09E−01 | −3.46E−01 | 2 | Unchanged Medium | |
| Q15584 | P03ab7 | heterogeneous nuclear ribonucle | 725.3651 | 569 | 858.2829 | 717.4409 | 2.43E−01 | −3.51E−01 | 2 | Unchanged Medium | |
| P12081 | J12ab4 | histidyl-tRNA synthetase | 555.5578 | 565 | 424.3904 | 514.8575 | −3.89E−01 | 2.34E−02 | 2 | Unchanged Medium | |
| Q9UBN7 | E11cd6 | histone deacetylase 6 | 685.5336 | 407 | 695.1552 | 595.8457 | 2.01E−02 | −7.53E−01 | 2 | Unchanged Medium | |
| Q13838 | M07cd4 | HLA-B associated transcript 1 | 681.4666 | 604 | 921.7532 | 735.5784 | 4.36E−01 | −1.75E−01 | 2 | Unchanged Medium | |
| Q99873 | H02ef7 | HMT1 hnRNP methyltransferase | 890.1154 | 836 | 1209.872 | 978.6584 | 4.43E−01 | −9.05E−02 | 2 | Unchanged Medium | |
| Q9H063 | M14gh8 | homolog of yeast MAF1 | 744.9951 | 921 | 1013.86 | 893.139 | 4.45E−01 | 3.05E−01 | 2 | Unchanged Medium | |
| P51610 | L21ab5 | host cell factor C1 (VP16-access | 583.7478 | 580 | 735.1691 | 632.9786 | 3.33E−01 | −9.25E−03 | 2 | Unchanged Medium | |
| O00165 | I04cd6 | HS1 binding protein | 1482.068 | 1490 | 2425.818 | 1800.489 | 7.11E−01 | 1.12E−02 | 2 | Unchanged Medium | |
| Q9Y2Q5 | P24ef7 | HSPC003 protein | 620.7783 | 1010 | 625.6574 | 752.165 | 1.13E−02 | 7.02E−01 | 2 | Unchanged Medium | |
| Q9Y2R0 | A05ef8 | HSPC009 protein | 1507.005 | 1720 | 1784.568 | 1668.909 | 2.44E−01 | 1.87E−01 | 2 | Unchanged Medium | |
| Q9Y2T0 | A17ef8 | HSPC022 protein | 453.0964 | 733 | 326.4215 | 504.1821 | −4.73E−01 | 6.94E−01 | 2 | Unchanged Medium | |
| Q9UNZ5 | C17ef8 | HSPC023 protein | 825.3888 | 1310 | 1347.488 | 1159.538 | 7.07E−01 | 6.62E−01 | 2 | Unchanged Medium | |
| Q9P032 | O19ef8 | HSPC125 protein | 558.8905 | 602 | 843.0953 | 668.1435 | 5.93E−01 | 1.08E−01 | 2 | Unchanged Medium | |
| Q9P019 | A06ef8 | HSPC141 protein | 717.9096 | 1700 | 1519.292 | 1312.254 | 1.08E+00 | 1.24E+00 | 2 | Unchanged Medium | |
| Q9P004 | A24ef8 | HSPC160 protein | 1182.006 | 1070 | 545.8523 | 932.7666 | −1.11E+00 | −1.43E−01 | 2 | Unchanged Medium | |
| Q9P003 | C04ef8 | HSPC163 protein | 801.5204 | 1410 | 1148.378 | 1118.827 | 5.19E−01 | 8.11E−01 | 2 | Unchanged Medium | |
| Q9P000 | C06ef8 | HSPC166 protein | 508.2074 | 921 | 523.3806 | 650.7428 | 4.24E−02 | 8.57E−01 | Unchanged Medium | | |
| Q9Y684 | N09ef2 | HSPC034 protein5 | 569.9533 | 628 | 797.4819 | 665.044 | 4.85E−01 | 1.39E−01 | 2 | Unchanged Medium | |
| Q9NRG2 | D01gh4 | HTGN29 protein | 1276.132 | 1470 | 1639.451 | 1462.8 | 3.61E−01 | 2.07E−01 | 2 | Unchanged Medium | |
| Q16775 | G15ab6 | hydroxyacyl glutathione hydrolas | 511.3412 | 676 | 407.0538 | 531.4885 | −3.29E−01 | 4.03E−01 | 2 | Unchanged Medium | |
| P40939 | D07ab3 | hydroxyacyl-Conezyme A dehyd | 1814.498 | 1770 | 1617.578 | 1733.575 | −1.66E−01 | −3.69E−02 | 2 | Unchanged Medium | |
| P55084 | D09ab3 | hydroxyacyl-Coenzyme A dehyd | 539.2691 | 890 | 1218.105 | 882.3271 | 1.18E+00 | 7.22E−01 | 2 | Unchanged Medium | |
| Q14526 | B13ab5 | hypermethylated in cancer 1 | 918.3678 | 904 | 1008.759 | 943.6552 | 1.35E+01 | −2.30E−02 | 2 | Unchanged Medium | |
| Q9UBS2 | A15ef1 | hyperpolarization activated cycli | 676.9289 | 688 | 520.6835 | 628.6277 | −3.79E−01 | 2.40E−02 | 2 | Unchanged Medium | |
| Q96EW8 | P08gh8 | hypothetical gene ZD52F10 | 727.4433 | 605 | 223.8014 | 518.5858 | −1.70E+00 | −2.67E−01 | 2 | Unchanged Medium | |
| Q9Y2S6 | D01ef2 | hypothetical protein | 1188.199 | 1490 | 1593.015 | 1423.596 | 4.23E−01 | 3.26E−01 | 2 | Unchanged Medium | |
| Q9UKZ1 | I23gh3 | hypothetical protein C40 | 657.2427 | 614 | 506.0272 | 592.3978 | −3.77E−01 | −9.84E−02 | 2 | Unchanged Medium | |
| Q9UJJ9 | D21gh8 | hypothetical protein CAB56184 | 432.491 | 695 | 601.25 | 576.3525 | 4.75E−01 | 6.85E−01 | 2 | Unchanged Medium | |
| O95891 | D08ef8 | hypothetical protein CL25022 | 1675.198 | 2070 | 1477.523 | 1741.841 | −1.81E−01 | 3.07E−01 | 2 | Unchanged Medium | |
| Q9UJI9 | F08gh6 | hypothetical protein DJ328E19.C | 1119.481 | 935 | 1005.691 | 1019.924 | −1.55E−01 | −2.60E−01 | 2 | Unchanged Medium | |
| Q9H0U3 | K20gh8 | hypothetical protein DKFZp564K | 1287.049 | 1080 | 1145.302 | 1169.228 | −1.68E−01 | −2.59E−01 | 2 | Unchanged Medium | |
| Q9NWD8 | N05gh2 | hypothetical protein FLJ10099 | 754.9838 | 758 | 710.6818 | 741.1753 | −8.68E−02 | 5.11E−03 | 2 | Unchanged Medium | |
| Q9NW90 | P21gh2 | hypothetical protein FLJ10211 | 492.5962 | 551 | 601.7235 | 548.3816 | 2.89E−01 | 1.61E−01 | 2 | Unchanged Medium | |
| Q9NW61 | B18gh2 | hypothetical protein FLJ10297 | 772.0358 | 844 | 620.8513 | 745.7235 | −3.14E−01 | 1.29E−01 | 2 | Unchanged Medium | |
| Q9NW16 | G04gh3 | hypothetical protein FLJ10374 | 756.2846 | 543 | 467.9722 | 589.0741 | −6.93E−01 | −4.78E−01 | 2 | Unchanged Medium | |
| Q9NVZ3 | G14gh3 | hypothetical protein FLJ10420 | 437.615 | 702 | 362.923 | 500.7932 | −2.70E−01 | 6.81E−01 | 2 | Unchanged Medium | |
| Q9NVC3 | D23gh3 | hypothetical protein FLJ10815 | 1402.842 | 928 | 598.595 | 976.6359 | −1.23E+00 | −5.95E−01 | 2 | Unchanged Medium | |
| Q9H9K7 | B10gh5 | hypothetical protein FLJ12681 | 446.1454 | 657 | 593.5606 | 565.6072 | 4.12E−01 | 5.59E−01 | 2 | Unchanged Medium | |
| Q9H8H4 | H06gh5 | hypothetical protein FLJ12800 | 1058.023 | 775 | 1421.894 | 1084.852 | 4.26E−01 | −4.50E−01 | 2 | Unchanged Medium | |
| Q9P0R5 | L02ef1 | hypothetical protein FLJ14868 | 702.1641 | 684 | 1117.168 | 834.4521 | 6.70E−01 | −3.78E−02 | 2 | Unchanged Medium | |
| Q9NXQ4 | M07gh3 | hypothetical protein FLJ20113 | 1503.987 | 1520 | 1545.141 | 1521.653 | 3.89E−02 | 1.13E−02 | 2 | Unchanged Medium | |
| Q9NX12 | M21gh3 | hypothetical protein FLJ20234 | 843.4985 | 877 | 176.0291 | 566.3149 | −1.85E+00 | 4.47E−01 | 2 | Unchanged Medium | |
| Q9NXD5 | E18gh2 | hypothetical protein FLJ20309 | 617.1482 | 1420 | 758.0334 | 932.5386 | 2.97E−01 | 1.20E+00 | 2 | Unchanged Medium | |
| Q9NX64 | I16gh2 | hypothetical protein FLJ20419 | 938.8956 | 825 | 981.6359 | 915.2399 | 6.42E−02 | −1.86E−01 | 2 | Unchanged Medium | |
| Q9NWX1 | A20gh3 | hypothetical protein FLJ20552 | 1366.306 | 1800 | 1533.352 | 1565.249 | 1.66E−01 | 3.95E−01 | 2 | Unchanged Medium | |
| Q9NWT8 | D11gh2 | hypothetical protein FLJ20608 | 980.6383 | 915 | 907.2393 | 934.2965 | −1.12E−01 | −9.99E−02 | 2 | Unchanged Medium | |
| Q9NWM3 | C20gh3 | hypothetical protein FL120739 | 381.202 | 593 | 689.893 | 554.671 | 8.56E−01 | 6.37E−01 | 2 | Unchanged Medium | |
| Q9H7C7 | D05gh5 | hypothetical protein FLJ21044 si | 551.5395 | 708 | 585.1487 | 615.0164 | 8.53E−02 | 3.61E−01 | 2 | Unchanged Medium | |
| Q9H6R6 | J23gh5 | hypothetical protein FLJ21952 | 425.3152 | 396 | 679.1125 | 500.0538 | 6.75E−01 | −1.04E−01 | 2 | Unchanged Medium | |
| Q9H673 | F11gh5 | hypothetical protein FLJ22548 si | 1394.825 | 1220 | 530.5646 | 1047.134 | −1.39E+00 | −1.98E−01 | 2 | Unchanged Medium | |
| O95476 | H15ef5 | hypothetical protein HSA011916 | 1310.097 | 1040 | 1465.664 | 1279.962 | 1.81E−01 | −3.27E−01 | 2 | Unchanged Medium | |
| Q9Y244 | B23ef2 | hypothetical protein HSPC014 | 1665.101 | 1530 | 1243.802 | 1481.658 | −4.23E−01 | −1.22E−01 | 2 | Unchanged Medium | |
| Q9P022 | P04ef2 | hypothetical protein HSPC138 | 666.1765 | 521 | 843.5979 | 610.0993 | −4.97E−02 | −3.56E−01 | 2 | Unchanged Medium | |
| Q9P013 | P06ef2 | hypothetical protein HSPC148 | 916.5214 | 1450 | 1133.279 | 1167.03 | 3.06E−01 | 6.63E−01 | 2 | Unchanged Medium | |
| Q9P009 | P14ef2 | hypothetical protein HSPC155 | 1031.658 | 1330 | 662.2481 | 1008.218 | −6.40E−01 | 3.67E−01 | 2 | Unchanged Medium | |
| Q9P0S9 | A19ef3 | hypothetical protein HSPC194 | 1336.172 | 1570 | 1957.055 | 1622.151 | 5.51E−01 | 2.36E−01 | 2 | Unchanged Medium | |
| Q9NPI0 | A23ef3 | hypothetical protein HSPC196 | 280.0356 | 644 | 672.086 | 532.0086 | 1.26E+00 | 1.20E+00 | 2 | Unchanged Medium | |
| O95887 | C18ef2 | hypothetical protein LOC51061 | 777.5591 | 709 | 551.8354 | 679.3596 | −4.95E−01 | −1.34E−01 | 2 | Unchanged Medium | |
| Q9P0T9 | K02ef2 | hypothetical protein LOC51234 | 1224.056 | 1290 | 1457.544 | 1322.295 | 2.52E−01 | 7.04E−02 | 2 | Unchanged Medium | |
| 09P0Q1 | K10ef2 | hypothetical protein LOC51248 | 376.8259 | 933 | 1014.441 | 774.623 | 1.43E+00 | 1.31E+00 | 2 | Unchanged Medium | |
| Q9P0P0 | K20ef2 | hypothetical protein LOC51255 | 1301.286 | 2190 | 1904.375 | 1798.292 | 5.49E−01 | 7.50E−01 | 2 | Unchanged Medium | |
| Q9P0N9 | K22ef2 | hypothetical protein LOC51256 | 540.326 | 374 | 604.1319 | 506.0883 | 1.61E−01 | −5.32E−01 | 2 | Unchanged Medium | |
| Q9NZD9 | M23ef2 | hypothetical protein LOC51323 | 600.1562 | 715 | 506.8479 | 607.2015 | −2.44E−01 | 2.52E−01 | 2 | Unchanged Medium | |
| O75207 | M02gh4 | hypothetical protein LOC57019 | 663.8685 | 644 | 680.7704 | 663.0017 | 3.63E−02 | −4.30E−02 | 2 | Unchanged Medium | |
| Q9BWL0 | L24gh7 | hypothetical protein MGC:5244; | 569.8856 | 436 | 732.9891 | 579.5932 | 3.63E−01 | −3.87E−01 | 2 | Unchanged Medium | |
| Q9BW61 | C09gh6 | hypothetical protein MGC2594 | 1446.395 | 2460 | 1354.586 | 1754.667 | −9.46E−02 | 7.68E−01 | 2 | Unchanged Medium | |
| Q9BQ49 | K05gh6 | hypothetical protein MGC2747 | 527.9287 | 679 | 677.2851 | 628.1009 | 3.59E−01 | 3.63E−01 | 2 | Unchanged Medium | |
| Q9BQD3 | E17gh6 | hypothetical protein MGC2749 | 841.5499 | 889 | 899.9017 | 876.8925 | 9.67E−02 | 7.95E−02 | 2 | Unchanged Medium | |
| Q9BQ61 | A09gh6 | hypothetical protein MGC2803 | 1234.395 | 2170 | 1980.421 | 1794.728 | 6.82E−01 | 8.13E−01 | 2 | Unchanged Medium | |
| Q9BTV4 | O23gh6 | hypothetical protein MGC3222 | 561.6572 | 502 | 583.5781 | 548.9637 | 5.52E−02 | −1.63E−01 | 2 | Unchanged Medium | |
| Q9BVX2 | C17gh6 | hypothetical protein MGC5576 | 747.2749 | 762 | 604.3725 | 704.7101 | −3.06E−01 | 2.91E−02 | 2 | Unchanged Medium | |
| Q9H773 | I17gh6 | hypothetical protein MGC5627 | 799.182 | 819 | 584.0079 | 734.1306 | −4.53E−01 | 3.57E−02 | 2 | Unchanged Medium | |
| Q9BPX5 | L02gh7 | hypothetical protein similar to act | 583.4076 | 424 | 632.4101 | 546.6359 | 1.16E−01 | −4.60E−01 | 2 | Unchanged Medium | |
| Q9UJX8 | B09ef3 | hypothetical protein; estradiol-in | 1184.334 | 948 | 1116.899 | 1083.179 | −8.46E−02 | −3.21E−01 | 2 Unchanged Medium | | |
| P00492 | P12cd6 | hypoxanthine phosphoribosyltra | 1210.895 | 233 | 566.0945 | 669.9437 | −1.10E+00 | −2.38E+00 | 2 Unchanged Medium | | |
| P22304 | I04ab6 | iduronate 2-sulfatase (Hunter syr | 1197.3 | 1320 | 1095.895 | 1205.085 | −1.28E−01 | 1.43E−01 | 2 | Unchanged Medium | |
| Q03827 | F02ef8 | immediate early protein | 650.6358 | 537 | 536.2705 | 574.4895 | −2.79E−01 | −2.78E−01 | 2 | Unchanged Medium | |
| P12268 | F11ab6 | IMP (inosine monophosphate) de | 1254.916 | 567 | 1293.693 | 1038.44 | 4.39E−02 | −1.15E+00 | 2 | Unchanged Medium | |
| Q9Y6K9 | P03cd3 | inhibitor of kappa light polypeptid | 822.3859 | 788 | 807.6888 | 805.9933 | −2.60E−02 | −6.18E−02 | 2 Unchanged Medium | | |
| Q14573 | F04ab6 | inositol 1;4;5-triphosphate recept | 555.9397 | 646 | 627.6143 | 609.9521 | 1.75E−01 | 2.17E−01 | 2 Unchanged Medium | | |
| O15357 | L09ab6 | inositol polyphosphate phosphat | 997.7512 | 1150 | 1290.473 | 1146.828 | 3.71E−01 | 2.08E−01 | 2 | Unchanged Medium | |
| O15503 | B05ab7 | insulin induced gene 1 | 472.0202 | 428 | 615.7553 | 505.2037 | 3.84E−01 | −1.42E−01 | 2 | Unchanged Medium | |
| P52945 | L13gh6 | insulin upstream factor 1 | 1437.285 | 3700 | 746.514 | 1961.135 | −9.45E−01 | 1.36E+00 | 2 | Unchanged Medium | |
| P51460 | B07ab7 | insulin-like 3 (Leydig cell) | 1227.459 | 1290 | 1040.851 | 1184.891 | −2.38E−01 | 6.76E−02 | 2 | Unchanged Medium | |
| P46977 | D20ab6 | integral membrane protein 1 | 1693.474 | 1790 | 2514.977 | 1998.711 | 5.71E−01 | 7.81E−02 | 2 | Unchanged Medium | |
| Q13418 | H08ef5 | integrin-linked kinase | 1340.442 | 962 | 1599.336 | 1300.502 | 2.55E−01 | −4.79E−01 | 2 | Unchanged Medium | |
| P48551 | O21ef7 | interferon (alpha: beta and omeg | 605.1613 | 669 | 389.178 | 554.33 | −6.37E−01 | 1.44E−01 | 2 | Unchanged Medium | |
| P15260 | A02ef7 | interferon gamma receptor 1 | 1165.764 | 2600 | 1179.083 | 1648.504 | 1.64E−02 | 1.16E+00 | 2 | Unchanged Medium | |
| P38484 | D12ef5 | interferon gamma receptor 2 (int | 593.8133 | 773 | 550.2678 | 638.9669 | −1.10E−01 | 3.80E−01 | 2 | Unchanged Medium | |
| O14896 | K05ef6 | interferon regulatory factor 6 | 495.9782 | 951 | 406.9438 | 617.9736 | −2.85E−01 | 9.39E−01 | 2 | Unchanged Medium | |
| | G13ef5 | interferon-gamma receptor (IFNC | 616.4461 | 474 | 457.8757 | 515.9592 | −4.29E−01 | −3.80E−01 | 2 | Unchanged Medium | |
| Q14116 | L03ab6 | interleukin 18 (interferon-gamma | 1525.444 | 1610 | 556.9614 | 1230.533 | −1.45E+00 | 7.71E−02 | 2 | Unchanged Medium | |
| O75874 | H11ab7 | isocitrate dehydrogenase 1 (NAD | 660.9773 | 496 | 904.897 | 687.2716 | 4.53E−01 | −4.14E−01 | 2 | Unchanged Medium | |
| P51553 | I21gh1 | isocitrate dehydrogenase 3 (NAD | 815.5318 | 899 | 784.1063 | 832.7908 | −5.67E−02 | 1.40E−01 | 2 | Unchanged Medium | |
| P41252 | P23ab6 | isoleucine-tRNA synthetase | 643.6728 | 316 | 992.267 | 650.6946 | 6.24E−01 | −1.03E+00 | 2 | Unchanged Medium | |
| Q9UE99 | J07ef7 | jagged 2 | 657.9935 | 460 | 696.5019 | 604.7722 | 8.21E−02 | −5.17E−01 | 2 | Unchanged Medium | |
| P52292 | J14ab6 | karyopherin alpha 2 (RAG cohor | 1105.911 | 1270 | 1670.406 | 1350.126 | 5.95E−01 | 2.04E−01 | 2 | Unchanged Medium | |
| P24390 | E07cd7 | KDEL (Lys-Asp-Glu-Leu) endopl | 1556.771 | 1210 | 1411.383 | 1392.342 | −1.41E−01 | −3.65E−01 | 2 | Unchanged Medium | |
| P08779 | D03ab7 | keratin 16 (focal non-epidermoly | 815.5106 | 786 | 663.0228 | 754.7055 | −2.99E−01 | −5.39E−02 | 2 | Unchanged Medium | |
| Q04695 | O24ab6 | keratin 17 | 580.3123 | 1180 | 1056.274 | 937.9159 | 8.64E−01 | 1.02E+00 | 2 | Unchanged Medium | |
| Q07666 | B04cd6 | KH domain containing; RNA bind | 1212.816 | 780 | 1103.092 | 1031.926 | −1.37E−01 | −6.37E−01 | 2 Unchanged Medium | | |
| Q14165 | O16gh1 | KIAA0152 gene product | 285.0232 | 348 | 1374.81 | 669.3755 | 2.27E+00 | 2.89E−01 | 2 | Unchanged Medium | |
| Q12765 | F03gh1 | KIAA0193 gene product | 951.658 | 1000 | 981.8527 | 979.0013 | 4.48E−02 | 7.68E−02 | 2 | Unchanged Medium | |
| O43310 | F11gh1 | KIAA0421 gene product | 482.1741 | 589 | 556.0999 | 542.3144 | 2.06E−01 | 2.88E−01 | 2 | Unchanged Medium | |
| O43167 | J03gh1 | KIAA0441 gene product | 1398.244 | 982 | 1102.589 | 1160.899 | −3.43E−01 | −5.10E−01 | 2 | Unchanged Medium | |
| Q9H7D5 | I18gh7 | KIAA1191 protein | 886.4026 | 1930 | 1061.302 | 1291.483 | 2.60E−01 | 1.12E+00 | 2 | Unchanged Medium | |
| Q9HD96 | L03cd8 | LAG1 longevity assurance homol | 1319.272 | 798 | 1016.572 | 1044.592 | −3.76E−01 | −7.25E−01 | 2 | Unchanged Medium | |
| O00182 | P04ab6 | lectin; galactoside-binding; solub | 760.6622 | 1300 | 527.6081 | 864.4149 | −5.28E−01 | 7.79E−01 | 2 | Unchanged Medium | |
| Q99538 | B04ef7 | legumain | 464.5 | 517 | 1105.037 | 695.3901 | 1.25E+00 | 1.53E−01 | 2 | Unchanged Medium | |
| O95751 | G01cd8 | leucine zipper: down-regulated i | 874.8344 | 274 | 970.9239 | 706.7407 | 1.50E−01 | −1.67E+00 | 2 | Unchanged Medium | |
| O15468 | P03ef4 | leukocyte immunoglobulin-like re | 826.8073 | 939 | 1120.204 | 962.0679 | 4.38E−01 | 1.84E−01 | 2 | Unchanged Medium | |
| Q9UBB4 | I06cd8 | like mouse brain protein E46 | 877.3997 | 623 | 692.5911 | 731.0054 | −3.41E−01 | −4.94E−01 | 2 | Unchanged Medium | |
| Q9UFW4 | N17ef8 | likely ortholog of rat golgi stackin | 1680.361 | 1700 | 2121.05 | 1834.238 | 3.36E−01 | 1.79E−02 | 2 | Unchanged Medium | |
| Q14847 | P09ab7 | LIM and SH3 protein 1 | 664.6143 | 758 | 895.1972 | 772.769 | 4.30E−01 | 1.91E−01 | 2 | Unchanged Medium | |
| O00158 | M09ef3 | LIM domain only 4 | 457.4088 | 299 | 1353.355 | 703.0924 | 1.56E+00 | −6.16E−01 | 2 | Unchanged Medium | |
| Q9HAP6 | M16gh5 | Lin-7b protein: likely ortholog of | 2114.229 | 1550 | 2175.236 | 1945.862 | 4.10E−02 | −4.50E−01 | 2 | Unchanged Medium | |
| P38571 | K08ab6 | lipase A: lysosomal acid: cholest | 604.0401 | 587 | 512.8879 | 568.1283 | −2.36E−01 | −4.02E−02 | 2 | Unchanged Medium | |
| Q99732 | Il8cd5 | LPS-induced TNF-alpha factor | 680.3876 | 893 | 449.4711 | 674.31 | −5.98E−01 | 3.92E−01 | 2 | Unchanged Medium | |
| Q9Y4Z1 | O21ef4 | Lsm3 protein | 1641.224 | 1300 | 1596.905 | 1511.517 | −3.95E−02 | −3.40E−01 | 2 | Unchanged Medium | |
| O75896 | D05cd7 | lung cancer candidate | 690.6061 | 772 | 601.3296 | 688.0098 | −2.00E−01 | 1.61E−01 | 2 | Unchanged Medium | |
| Q06643 | O07ef6 | lymphotoxin beta (TNF superfam | 405.7368 | 1040 | 298.9254 | 582.7217 | −4.41E−01 | 1.36E+00 | 2 | Unchanged Medium | |
| O95372 | J04ef7 | lysophospholipase II | 480.0562 | 737 | 752.7079 | 656.485 | 6.49E−01 | 6.18E−01 | 2 | Unchanged Medium | |
| P13473 | N06ab6 | lysosomal-associated membrane | 952.6393 | 2000 | 1277.773 | 1408.92 | 4.24E−01 | 1.07E+00 | 2 | Unchanged Medium | |
| Q08397 | F09ab7 | lysyl oxidase-like 1 | 1391.453 | 1630 | 2243.025 | 1753.531 | 6.89E−01 | 2.25E−01 | 2 | Unchanged Medium | |
| Q15046 | B17ab7 | lysyl-tRNA synthetase | 900.8177 | 556 | 539.813 | 665.5485 | −7.39E−01 | −6.96E−01 | 2 | Unchanged Medium | |
| Q13312 | D17cd3 | MAD1 mitotic arrest deficient-like | 807.7267 | 503 | 574.4266 | 628.231 | −4.92E−01 | −6.85E−01 | 2 | Unchanged Medium | |
| Q09160 | A05ab6 | major histocompatibitity complex: | 1524.076 | 2710 | 1290.783 | 1841.106 | −2.40E−01 | 8.30E−01 | 2 | Unchanged Medium | |
| P40925 | J23ab7 | malate dehydrogenase 1; NAD (1 | 1144.218 | 788 | 1089.134 | 1007.157 | −7.12E−02 | −5.38E−01 | 2 | Unchanged Medium | |
| P40926 | L01ab7 | malate dehydrogenase 2; NAD ( | 1525.837 | 1020 | 1212.544 | 1251.158 | −3.32E−01 | −5.88E−01 | 2 | Unchanged Medium | |
| Q16626 | J15ef3 | male-enhanced antigen | 1253.21 | 1530 | 1827.301 | 1536.52 | 5.44E−01 | 2.87E−01 | 2 | Unchanged Medium | |
| Q9NQG1 | C06gh5 | mannosidase: beta A: lysosomal | 533.2883 | 800 | 736.627 | 689.9075 | 4.86E−01 | 5.85E−01 | 2 | Unchanged Medium | |
| P26572 | G11ab7 | mannosyl (alpha-1;3-)-glycoprote | 619.6733 | 369 | 887.6535 | 625.3732 | 5.18E−01 | −7.49E−01 | 2 | Unchanged Medium | |
| Q10469 | G15ab7 | mannosyl (alpha-1;6-)-glycoprote | 600.1049 | 583 | 636.621 | 606.4146 | 8.52E−02 | −4.29E−02 | 2 | Unchanged Medium | |
| P41223 | L09cd4 | maternal G10 transcript | 372.2443 | 563 | 575.7695 | 503.5179 | 6.29E−01 | 5.96E−01 | 2 | Unchanged Medium | |
| Q9Y5V3 | G13cd6 | melanoma antigen: family D: 1 | 397.4495 | 952 | 1674.881 | 1008.268 | 2.08E+00 | 1.26E+00 | 2 | Unchanged Medium | |
| P43121 | I22ef5 | melanoma cell adhesion molecul 886.0756 | 795 | 432.1718 | 704.4837 | −1.04E+00 | −1.56E−01 | 2 | Unchanged Medium | | |
| Q92494 | D11ef1 | membrane cofactor protein (CD4 | 973.362 | 1050 | 834.044 | 952.9144 | −2.23E−01 | 1.11E−01 | 2 | Unchanged Medium | |
| Q14444 | H15ab7 | membrane component: chromos | 549.4803 | 666 | 464.8174 | 560.0583 | −2.41E−01 | 2.77E−01 | 2 | Unchanged Medium | |
| Q9NPE2 | O12ef2 | mesenchymal stem cell protein D | 749.8938 | 932 | 1085.26 | 922.5469 | 5.33E−01 | 3.14E−01 | 2 | Unchanged Medium | |
| Q92571 | G07ab7 | mesoderm specific transcript hom | 617.561 | 446 | 537.1735 | 533.4868 | −2.01E−01 | −4.71E−01 | 2 | Unchanged Medium | |
| O95204 | N11ef3 | metalloprotease 1 (pitrilysin famil | 735.157 | 349 | 478.8153 | 520.899 | −6.19E−01 | −1.08E+00 | 2 | Unchanged Medium | |
| P80297 | H20ab7 | metallothionein 1L | 569.6327 | 502 | 656.4441 | 582.747 | 1.55E−01 | −2.32E−01 | 2 | Unchanged Medium | |
| Q99735 | C18ef7 | microsomal glutathione S-transfe | 527.4698 | 686 | 373.9779 | 529.1741 | −4.96E−01 | 3.79E−01 | 2 | Unchanged Medium | |
| Q9BXW5 | D13gh8 | microtubule-associated protein 1 | 247.2817 | 692 | 887.6299 | 609.0447 | 1.64E+00 | 1.49E+00 | 2 | Unchanged Medium | |
| Q9Y6C9 | M14ef3 | mitochondrial carrier homolog 2 | 1874.693 | 1910 | 1893.062 | 1892.145 | 1.39E−02 | 2.56E−02 | 2 | Unchanged Medium | |
| Q9Y3B7 | E07ef2 | mitochondrial ribosomal protein L | 401.0368 | 421 | 732.113 | 517.9208 | 8.68E−01 | 6.88E−02 | 2 | Unchanged Medium | |
| Q9P015 | A12ef8 | mitochondrial ribosomal protein L | 553.658 | 760 | 569.0617 | 634.2498 | 8.94E−02 | 4.57E−01 | 2 | Unchanged Medium | |
| Q9NX20 | M06gh2 | mitochondrial ribosomal protein L | 642.9143 | 496 | 701.1442 | 613.4293 | 1.25E−01 | −3.74E−01 | 2 | Unchanged Medium | |
| Q9H0U6 | O13ef8 | milochondrial ribosomal protein L | 741.5383 | 1000 | 826.8816 | 856.3674 | 1.57E−01 | 4.32E−01 | 2 | Unchanged Medium | |
| Q9NZE8 | O04ef2 | mitochondrial ribosomal protein L | 914.9722 | 910 | 956.7888 | 927.2731 | 6.45E−02 | −7.77E−03 | 2 | Unchanged Medium | |
| Q9P0P3 | K18ef2 | mitochondrial ribosomal protein L | 1217.101 | 891 | 1196.143 | 1101.306 | −2.51E−02 | −4.50E−01 | 2 | Unchanged Medium | |
| Q9Y317 | M15ef2 | mitochondrial ribosomal protein L | 799.6979 | 1020 | 1372.352 | 1063.499 | 7.79E−01 | 3.49E−01 | 2 | Unchanged Medium | |
| Q9Y6G3 | C19ef8 | mitochondrial ribosomal protein L | 571.7264 | 810 | 689.3229 | 690.2211 | 2.70E−01 | 5.02E−01 | 2 | Unchanged Medium | |
| Q9BYC7 | K12gh8 | mitochondrial ribosomal protein L | 490.5463 | 665 | 619.0928 | 591.6118 | 3.36E−01 | 4.39E−01 | 2 | Unchanged Medium | |
| Q9Y3D3 | B12ef1 | mitochondrial ribosomal protein S | 966.7611 | 1020 | 1006.958 | 997.1495 | 5.88E−02 | 7.41E−02 | 2 | Unchanged Medium | |
| Q9Y676 | C15ef8 | mitochondrial ribosomal protein S | 683.2889 | 580 | 761.9887 | 674.9976 | 1.57E−01 | −2.37E−01 | 2 | Unchanged Medium | |
| Q9Y3D5 | G19ef2 | mitochondrial ribosomal protein S | 696.3306 | 1080 | 825.557 | 865.8021 | 2.46E−01 | 6.27E−01 | 2 | Unchanged Medium | |
| P82921 | O11gh7 | mitochondrial ribosomal protein S | 748.082 | 1190 | 1115.5 | 1019.33 | 5.76E−01 | 6.75E−01 | 2 | Unchanged Medium | |
| P52650 | D07gh4 | mitochondrial ribosomal protein S | 554.1784 | 515 | 470.683 | 513.1021 | −2.36E−01 | −1.07E−01 | 2 | Unchanged Medium | |
| Q9Y2Q9 | A03ef8 | mitochondrial ribosomal protein S | 1439.076 | 1420 | 1329.749 | 1395.534 | −1.14E−01 | −2.15E−02 | 2 | Unchanged Medium | |
| Q9NP92 | F02ef3 | mitochondrial ribosomal protein S | 669.1652 | 532 | 617.3223 | 606.105 | −1.16E−01 | −3.31E−01 | 2 | Unchanged Medium | |
| Q02750 | J08ef5 | mitogen-activated protein kinase | 615.689 | 701 | 453.8702 | 590.1129 | −4.40E−01 | 1.87E−01 | 2 | Unchanged Medium | |
| Q02779 | K09ab7 | mitogen-activated protein kinase | 597.1743 | 423 | 509.6637 | 510.095 | −2.29E−01 | −4.96E−01 | 2 | Unchanged Medium | |
| Q12851 | N10ab8 | mitogen-activated protein kinase | 988.0971 | 1020 | 976.7391 | 994.1804 | −1.67E−02 | 4.26E−02 | 2 | Unchanged Medium | |
| P49137 | J06ef5 | mitogen-activated protein kinase | 942.7459 | 1000 | 1068.579 | 1004.624 | 1.81E−01 | 8.87E−02 | 2 | Unchanged Medium | |
| Q9BYG3 | O02gh8 | MKI67 (FHA domain) interacting | 791.8688 | 407 | 307.4404 | 502.0002 | −1.36E+00 | −9.61E−01 | 2 | Unchanged Medium | |
| Q15014 | C17cd8 | MORF-related gene X | 1146.48 | 1030 | 1262.273 | 1144.777 | 1.39E−01 | −1.61E−01 | 2 | Unchanged Medium | |
| | F22ef4 | mRNA: clone:PO2ST9 | 1052.442 | 1040 | 905.2614 | 999.5166 | −2.17E−01 | −1.60E−02 | 2 | Unchanged Medium | |
| Q15773 | M18ef5 | myeloid leukemia factor 2 | 577.4585 | 467 | 766.6809 | 603.749 | 4.09E−01 | −3.06E−01 | 2 | Unchanged Medium | |
| P35579 | O01ef1 | myosin: heavy polypeptide 9: no | 1426.003 | 1150 | 1203.964 | 1261.007 | −2.44E−01 | −3.07E−01 | 2 | Unchanged Medium | |
| Q9Y6D2 | C21ef2 | N-acetyltransferase 5 (ARD1 hor | 531.5485 | 708 | 274.9715 | 504.9319 | −9.51E−01 | 4.14E−01 | 2 | Unchanged Medium | |
| P41227 | G13gh1 | N-acetyltransferase; homolog of | 800.6676 | 905 | 815.2554 | 841.2057 | 3.13E−02 | 1.76E−01 | 2 | Unchanged Medium | |
| Q13510 | H20ab2 | N-acylsphingosine amidohydrola | 752.1534 | 1660 | 677.5006 | 1028.456 | −1.51E−01 | 1.14E+00 | 2 | Unchanged Medium | |
| O43678 | O09ab7 | NADH dehydrogenase (ubiquino | 1366.754 | 1930 | 2137.906 | 1810.699 | 6.45E−01 | 4.96E−01 | 2 | Unchanged Medium | |
| O95167 | E24ab7 | NADH dehydrogenase (ubiquino | 1431.655 | 1770 | 1506.934 | 1570.238 | 7.39E−02 | 3.08E−01 | 2 | Unchanged Medium | |
| P56556 | O11ab7 | NADH dehydrogenase (ubiquino | 1650.612 | 916 | 795.8725 | 1120.973 | −1.05E+00 | −8.49E−01 | 2 | Unchanged Medium | |
| O95182 | K06ab7 | NADH dehydrogenase (ubiquino | 750.0893 | 777 | 725.1711 | 750.6457 | −4.87E−02 | 5.03E−02 | 2 | Unchanged Medium | |
| P51970 | L04cd8 | NADH dehydrogenase (ubiquino | 554.1995 | 728 | 1021.163 | 767.6925 | 8.82E−01 | 3.93E−01 | 2 | Unchanged Medium | |
| O96000 | J10ab8 | NADH dehydrogenase (ubiquino | 1110.197 | 1440 | 1111.615 | 1219.156 | 1.84E−03 | 3.71E−01 | 2 | Unchanged Medium | |
| O95178 | J06ab8 | NADH dehydrogenase (ubiquino | 925.3926 | 768 | 815.266 | 836.3224 | −1.83E−01 | −2.68E−01 | 2 | Unchanged Medium | |
| O95139 | M03ab8 | NADH dehydrogenase (ubiquino | 1392.873 | 1480 | 2162.279 | 1678.472 | 6.34E−01 | 8.78E−02 | 2 | Unchanged Medium | |
| P17568 | H02ab8 | NADH dehydrogenase (ubiquinn | 787.0681 | 567 | 591.1924 | 648.3703 | −4.13E−01 | −4.74E−01 | 2 | Unchanged Medium | |
| Q9Y6M9 | P16ab8 | NADH dehydrogenase (ubiquino | 1299.803 | 1450 | 746.8017 | 1164.524 | −7.99E−01 | 1.55E−01 | 2 | Unchanged Medium | |
| O43677 | M05ab8 | NADH dehydrogenase (ubiquino | 586.5059 | 601 | 532.551 | 573.2205 | −1.39E−01 | 3.43E−02 | 2 | Unchanged Medium | |
| O00217 | M09ab8 | NADH dehydrogenase (ubiquino | 1383.411 | 1130 | 1194.001 | 1234.615 | −2.12E−01 | −2.96E−01 | 2 | Unchanged Medium | |
| P19404 | M07ef1 | NADH dehydrogenase (ubiquino | 1151.224 | 443 | 356.308 | 650.0945 | −1.69E+00 | −1.38E+00 | 2 | Unchanged Medium | |
| O76008 | A12cd6 | Nef-associated factor 1 | 720.2744 | 951 | 424.04 | 698.4474 | −7.64E−01 | 4.01E−01 | 2 | Unchanged Medium | |
| Q9UPY4 | K22ef3 | nesca protein | 425.9149 | 469 | 621.9336 | 505.6782 | 5.46E−01 | 1.40E−01 | 2 | Unchanged Medium | |
| | L19gh8 | neurabin II | 1241.544 | 862 | 1023.077 | 1042.081 | −2.79E−01 | −5.27E−01 | 2 | Unchanged Medium | |
| Q99742 | O07ab8 | neuronal PAS domain protein 1 | 1076.259 | 405 | 606.3589 | 695.8521 | −8.28E−01 | −1.41E+00 | 2 | Unchanged Medium | |
| O60448 | P20ef3 | neuronal thread protein | 1831.592 | 1660 | 1490.624 | 1662.051 | −2.97E−01 | −1.38E−01 | 2 | Unchanged Medium | |
| Q9UNW9 | O05ab8 | neuro-oncological ventral antige | 1049.959 | 1050 | 1219.792 | 1106.962 | 2.16E−01 | 1.70E−03 | 2 | Unchanged Medium | |
| Q9UGL9 | C09gh3 | NICE-1 protein | 1026.838 | 1360 | 381.058 | 923.3423 | −1.43E+00 | 4.08E−01 | 2 | Unchanged Medium | |
| Q9UGL6 | M09ef4 | NICE-5 protein | 912.7542 | 1390 | 955.016 | 1086.502 | 6.53E−02 | 6.09E−01 | 2 | Unchanged Medium | |
| Q92982 | I09ef7 | ninjurin 1 | 458.23 | 689 | 412.0181 | 519.6952 | −1.53E−01 | 5.88E−01 | 2 | Unchanged Medium | |
| Q9BPW8 | N19cd3 | nipsnap homolog 1 (C. elegans) | 829.4266 | 529 | 562.9689 | 640.3075 | −5.59E−01 | −6.50E−01 | 2 | Unchanged Medium | |
| Q9NQR4 | B13gh4 | Nit protein 2 | 519.1587 | 868 | 665.8632 | 684.4701 | 3.59E−01 | 7.42E−01 | 2 | Unchanged Medium | |
| Q9UF10 | L08ef2 | non-canonical ubquitin conjugatin | 1336.406 | 2000 | 1488.008 | 1608.811 | 1.55E−01 | 5.83E−01 | 2 | Unchanged Medium | |
| Q9Y385 | H11ef2 | non-canonical ubquitin conjugatin | 625.6356 | 808 | 669.812 | 701.1992 | 9.84E−02 | 3.69E−01 | 2 | Unchanged Medium | |
| Q15233 | O02ef1 | non-POU domain containing; oct | 1083.363 | 1210 | 1234.216 | 1174.503 | 1.88E−01 | 1.55E−01 | 2 | Unchanged Medium | |
| Q13137 | A03cd6 | nuclear domain 10 protein | 910.7725 | 914 | 944.6079 | 923.1712 | 5.26E−02 | 5.31E−03 | 2 | Unchanged Medium | |
| Q16236 | M03cd1 | nuclear factor (erythroid-derived | 1233.751 | 1180 | 1043.638 | 1153.088 | −2.41E−01 | −6.20E−02 | 2 | Unchanged Medium | |
| P08651 | B07ef1 | nuclear factor I/C (CCAAT-bindin | 726.0476 | 624 | 659.6305 | 669.8575 | −1.38E−01 | −2.19E−01 | 2 | Unchanged Medium | |
| O95134 | P10cd3 | nuclear localization signal delete | 465.2933 | 522 | 536.7333 | 508.0938 | 2.06E−01 | 1.67E−01 | 2 | Unchanged Medium | |
| Q15772 | N19ab2 | nuclear protein: marker for differe | 1473.704 | 923 | 1157.202 | 1184.738 | −3.49E−01 | −6.75E−01 | 2 | Unchanged Medium | |
| Q9UHY1 | L15cd8 | nuclear receptor binding protein | 515.949 | 948 | 619.7512 | 694.4539 | 2.64E−01 | 8.77E−01 | 2 | Unchanged Medium | |
| Q9UPC9 | H23ab2 | nuclear receptor coactivator 3 | 822.7768 | 1190 | 932.9058 | 982.77 | 1.51E−01 | 5.36E−01 | 2 | Unchanged Medium | |
| Q15325 | N22cd2 | nuclease sensitive element bindi | 1644.614 | 1720 | 2064.24 | 1808.77 | 3.28E−01 | 6.25E−02 | 2 | Unchanged Medium | |
| O00567 | D05cd6 | nucleolar protein 5A (56kD with | 659.4335 | 608 | 623.2536 | 630.2325 | −8.14E−02 | −1.17E−01 | 2 | Unchanged Medium | |
| Q9NX24 | A10gh3 | nucleolar protein family A: memb | 466.2539 | 625 | 594.8443 | 561.9547 | 3.51E−01 | 4.22E−01 | 2 | Unchanged Medium | |
| P06748 | K06gh6 | nucleophosmin (nucleolar phosp | 1605.368 | 1080 | 1136.972 | 1275.358 | −4.98E−01 | −5.67E−01 | 2 | Unchanged Medium | |
| P50583 | B01ab2 | nudix (nucleoside diphosphate li | 1590.763 | 211 | 251.3768 | 684.354 | −2.66E+00 | −2.91E+00 | 2 | Unchanged Medium | |
| Q9UHM6 | N20gh8 | opsin 4 (melanopsin) | 1076.73 | 1200 | 1643.887 | 1305.239 | 6.10E−01 | 1.50E−01 | 2 | Unchanged Medium | |
| Q9Y218 | P22gh1 | optineurin | 1014.697 | 1980 | 912.1917 | 1302.523 | −1.54E−01 | 9.66E−01 | 2 | Unchanged Medium | |
| Q16612 | O01cd5 | P311 protein | 322.7444 | 349 | 2231.846 | 967.7934 | 2.79E+00 | 1.12E−01 | 2 | Unchanged Medium | |
| P55771 | J21cd7 | paired box gene 9 | 692.506 | 380 | 573.135 | 548.4374 | −2.73E−01 | −8.67E−01 | 2 | Unchanged Medium | |
| Q9UKJ1 | N21cd8 | paired immunoglobulin-like recep | 674.1862 | 450 | 558.3225 | 560.9087 | −2.72E−01 | −5.83E−01 | 2 | Unchanged Medium | |
| Q15165 | P24ab7 | paraoxonase 2 | 338.5244 | 891 | 583.7771 | 604.4337 | 7.86E−01 | 1.40E+00 | 2 | Unchanged Medium | |
| Q07002 | P06ef5 | PCTAIRE protein kinase 3 | 836.5659 | 916 | 632.7298 | 795.1858 | −4.03E−01 | 1.31E−01 | 2 | Unchanged Medium | |
| Q9Y3C6 | J07ef2 | peptidylprolyl isomerase (cyclop | 548.5938 | 731 | 526.6097 | 602.0621 | −5.90E−02 | 4.14E−01 | 2 | Unchanged Medium | |
| P30405 | F14cd5 | peptidylprolyl isomerase F (cyclo | 843.1834 | 925 | 467.6738 | 745.2396 | −8.50E−01 | 1.33E−01 | 2 | Unchanged Medium | |
| O43924 | G24ab8 | phosphodiesterase 6D; cGMP-sp | 373.1208 | 630 | 699.1887 | 567.4949 | 9.06E−01 | 7.56E−01 | 2 | Unchanged Medium | |
| P17858 | K10ab8 | phosphofructokinase; liver | 950.5746 | 525 | 774.727 | 750.1835 | −2.95E−01 | −8.56E−01 | 2 | Unchanged Medium | |
| Q01813 | C17ef1 | phosphofructokinase; platelet | 573.5211 | 646 | 555.9236 | 591.6879 | −4.50E−02 | 1.71E−01 | 2 | Unchanged Medium | |
| O00511 | N20cd3 | phosphoprotein enriched in astro | 1330.005 | 1500 | 2288.098 | 1704.656 | 7.83E−01 | 1.70E−01 | 2 | Unchanged Medium | |
| P11216 | B16ab8 | phosphorylase: glycogen: brain | 516.7884 | 495 | 988.163 | 666.5476 | 9.35E−01 | −6.30E−02 | 2 | Unchanged Medium | |
| P00749 | P19ef7 | plasminogen activator; urokinase | 1096.052 | 3440 | 1051.513 | 1861.568 | −5.98E−02 | 1.65E+00 | 2 | Unchanged Medium | |
| P13797 | C13cd1 | plastic 3 (T isoform) | 990.1173 | 804 | 1393.037 | 1062.438 | 4.93E−01 | −3.00E−01 | 2 | Unchanged Medium | |
| Q15102 | E16ab8 | platelet-activating factor acetylhy | 469.9778 | 674 | 468.9765 | 537.6672 | −3.08E−03 | 5.20E−01 | 2 | Unchanged Medium | |
| P01127 | A08ef5 | platelet-derived growth factor bel | 500.6466 | 674 | 456.4578 | 543.5869 | −1.33E−01 | 4.28E−01 | 2 | Unchanged Medium | |
| Q15795 | C19ef6 | pleckstrin homology; Sec7 and c | 578.295 | 740 | 518.8327 | 612.22 | −1.57E−01 | 3.55E−01 | 2 | Unchanged Medium | |
| O43660 | O18ab8 | pleiotropic regulator 1 (PRL 1hom | 743.7263 | 933 | 804.3456 | 827.0995 | 1.13E−01 | 3.27E−01 | 2 | Unchanged Medium | |
| Q15155 | G12ef3 | pM5 protein | 664.2511 | 423 | 490.295 | 525.8869 | −4.38E−01 | −6.51E−01 | 2 | Unchanged Medium | |
| Q9H361 | L04gh7 | poly(A) binding protein: cytoplas | 759.4566 | 491 | 710.9543 | 653.8286 | −9.52E−02 | −6.29E−01 | 2 | Unchanged Medium | |
| Q15366 | A11cd1 | poly(rC) binding protein 2 | 1951.241 | 1490 | 2128.S85 | 1857.745 | 1.26E−01 | −3.86E−01 | 2 | Unchanged Medium | |
| P19388 | B15ab8 | polymerase (RHA) II (DNA direct | 552.3545 | 529 | 698.4965 | 593.4201 | 3.39E−01 | −6.12E−02 | 2 | Unchanged Medium | |
| P52434 | F05gh6 | polymerase (RNA) II (DNA direct | 949.7064 | 1480 | 1210.411 | 1211.761 | 3.50E−01 | 6.35E−01 | 2 | Unchanged Medium | |
| P36954 | A20cd1 | polymerase (RNA) II (DNA direct | 1603.557 | 1820 | 1413.573 | 1613.004 | −1.82E−01 | 1.84E−01 | 2 | Unchanged Medium | |
| P53803 | C17cd1 | polymerase (RNA) II (DNA direct | 437.9544 | 893 | 528.0144 | 619.6184 | 2.70E−01 | 1.03E+00 | 2 | Unchanged Medium | |
| Q9UQQ3 | J08cd6 | POP4 (processing of precursor: | 450.5848 | 645 | 551.3661 | 558.9966 | 1.98E−01 | 4.25E−01 | 2 | Unchanged Medium | |
| Q13670 | B07ab6 | postmeiotic segregation increase | 812.7865 | 779 | 532.0548 | 708.0473 | −6.11E−01 | −6.07E−02 | 2 | Unchanged Medium | |
| Q9P0J7 | L07gh4 | potassium channel modulatory fa | 1401.591 | 1720 | 1192.204 | 1438.482 | −2.33E−01 | 2.97E−01 | 2 | Unchanged Medium | |
| O60925 | K02ab8 | prefoldln 1 | 1631.996 | 1720 | 1660.632 | 1737.289 | 1.89E−01 | 7.51E−02 | 2 | Unchanged Medium | |
| Q9UHV9 | J15ef4 | prefoldin 2 | 514.4897 | 882 | 833.1525 | 743.3158 | 6.95E−01 | 7.78E−01 | 2 | Unchanged Medium | |
| Q13519 | J07ef6 | prepronociceptin | 564.2079 | 464 | 887.4522 | 638.4071 | 6.53E−01 | −2.83E−01 | 2 | Unchanged Medium | |
| P04156 | A13ab8 | prion protein (p27-30) (CreutzfeL | 797.9495 | 631 | 701.8217 | 710.1438 | −1.85E−01 | −3.39E−01 | 2 | Unchanged Medium | |
| Q9UI73 | K03ef8 | PRO0246 protein | 430.412 | 535 | 546.8599 | 504.0018 | 3.45E−01 | 3.13E−01 | 2 | Unchanged Medium | |
| Q9P0T3 | D14ef1 | proapoptotic caspase adaptor pr | 1261.463 | 1650 | 2553.652 | 1820.221 | 1.02E+00 | 3.83E−01 | 2 | Unchanged Medium | |
| Q02809 | P20ab7 | procollagen-lysine; 2-oxoglutarat | 503.7557 | 477 | 1993.232 | 991.2271 | 1.98E+00 | −7.97E−02 | 2 | Unchanged Medium | |
| P07237 | G13ab8 | procollagen-proline; 2-oxoglutara | 633.8309 | 406 | 575.6835 | 538.3489 | −1.39E−01 | −6.44E−01 | 2 | Unchanged Medium | |
| P09466 | E12ab8 | progestagen-associated endome | 387.0939 | 625 | 708.8717 | 567.0247 | 9.49E−01 | 7.68E−01 | 2 | Unchanged Medium | |
| Q16342 | G22ef6 | programmed cell death 2 | 735.0961 | 759 | 490.3938 | 661.3332 | −5.84E−01 | 4.52E−02 | 2 | Unchanged Medium | |
| O75340 | I02cd8 | programmed cell death 6 | 1055.886 | 1090 | 1356.597 | 1167.762 | 3.62E−01 | 4.70E−02 | 2 | Unchanged Medium | |
| P35232 | C22ef5 | prohibitin | 2045.693 | 1810 | 1600.236 | 1818.121 | −3.54E−01 | −1.78E−01 | 2 | Unchanged Medium | |
| Q9UQ80 | O03cd1 | proliferation-associated 2G4: 38 | 1902.387 | 1390 | 2003.135 | 1763.59 | 7.446−02 | −4.58E−01 | 2 | Unchanged Medium | |
| Q12796 | E05ab3 | proline rich 2 | 1927.78 | 1530 | 1475.973 | 1643.691 | −3.85E−01 | −3.36E−01 | 2 | Unchanged Medium | |
| Q15188 | P20cd2 | proline-rich protein BstNI subfam | 997.5582 | 1290 | 1404.4 | 1232.034 | 4.93E−01 | 3.76E−01 | 2 | Unchanged Medium | |
| P35998 | L09ab8 | proteasome (prosome; macropai | 1010.463 | 1200 | 1059.594 | 1090.401 | 6.65E−02 | 2.49E−01 | 2 | Unchanged Medium | |
| P47210 | N16cd2 | proteasome (prosome; macropai | 1037.339 | 1150 | 1193 | 1127.919 | 2.02E−01 | 1.53E−01 | 2 | Unchanged Medium | |
| O00495 | N03ab8 | protessome (prosome; macropai | 1043.875 | 498 | 563.7775 | 701.7628 | −8.89E−01 | −1.07E+00 | 2 | Unchanged Medium | |
| O75831 | N07ab8 | proteasome (prosome; macropai | 763.7632 | 622 | 506.8297 | 630.7237 | −5.92E−01 | −2.97E−01 | 2 | Unchanged Medium | |
| Q13200 | L15ab8 | proteasome (prosome; macropai | 644.3008 | 594 | 331.4288 | 523.3702 | −9.59E−01 | −1.16E−01 | 2 | Unchanged Medium | |
| O43242 | L17ab8 | proteasome (prosome; macropai | 2159.327 | 1230 | 1637.67 | 1675.386 | −3.99E−01 | −8.13E−01 | 2 | Unchanged Medium | |
| P55036 | N18cd2 | proteasome (prosome; macropai | 460.8093 | 724 | 588.5346 | 591.0419 | 3.53E−Q1 | 6.51E−01 | 2 | Unchanged Medium | |
| P48556 | L21ab8 | proteasome (prosome; macropai | 1240.243 | 1030 | 1685.116 | 1317.42 | 4.42E−01 | −2.72E−01 | 2 | Unchanged Medium | |
| Q92530 | G07cd6 | proteasome (prosome; macropai | 1804.777 | 1570 | 1800.731 | 1724.253 | −3.246−03 | −2.04E−01 | 2 | Unchanged Medium | |
| P28066 | J13ab8 | proteasome (prosome; macropai | 766.589 | 767 | 775.2704 | 769.7417 | 1.62E−02 | 1.46E−03 | 2 | Unchanged Medium | |
| O14818 | J15ab5 | proteasome (prosome; macropai | 1242.517 | 1540 | 1139.994 | 1308.162 | −1.24E−01 | 3.12E−01 | 2 | Unchanged Medium | |
| P49721 | J19ab8 | proteasome (prosome; macropai | 1832.921 | 2110 | 1617.062 | 1853.69 | −1.81E−01 | 2.04E−01 | 2 | Unchanged Medium | |
| P28062 | H18ab8 | proteasome (prosome; macropai | 797.3912 | 905 | 505.3638 | 735.864 | −6.58E−01 | 1.82E−01 | 2 | Unchanged Medium | |
| P28065 | B06ef7 | proteasome (prosome; macropai | 771.1998 | 1630 | 1002.72 | 1133.626 | 3.79E−01 | 1.08E+00 | 2 | Unchanged Medium | |
| P10619 | A07ab8 | protective protein for beta-galact | 1539.157 | 907 | 980.3046 | 1142.279 | −6.51E−01 | −7.62E−01 | 2 | Unchanged Medium | |
| Q9UNN8 | F03cd6 | protein C receptor: endothelial (E | 892.1589 | 702 | 488.5084 | 694.2014 | −8.69E−01 | −3.46E−01 | 2 | Unchanged Medium | |
| Q15084 | H02cd5 | protein disulfide isomerase-relat | 1081.309 | 407 | 859.0228 | 782.4339 | −3.32E−01 | −1.41E+00 | 2 | Unchanged Medium | |
| Q9Y2B9 | C22cd7 | protein kinase (cAMP-dependent | 650.9588 | 1040 | 754.6101 | 814.4047 | 2.13E−01 | 6.73E−01 | 2 | Unchanged Medium | |
| Q13517 | A09ef5 | protein kinase C binding protein | 380.9448 | 587 | 630.5497 | 532.9442 | 7.27E−01 | 6.25E−01 | 2 | Unchanged Medium | |
| P54619 | F15ab8 | protein kinase; AMP-activated; g | 482.8359 | 572 | 841.1746 | 632.0978 | 8.01E−01 | 2.45E−01 | 2 | Unchanged Medium | |
| P17612 | F13ab8 | protein kinase; cAMP-dependent | 643.0385 | 629 | 901.5293 | 724.5168 | 4.87E−01 | −3.19E−02 | 2 | Unchanged Medium | |
| P09131 | F10gh1 | Protein P3 | 529.0069 | 607 | 617.0621 | 564.2407 | 2.22E−01 | 1.98E−01 | 2 | Unchanged Medium | |
| O60927 | P02gh1 | protein phosphatase 1; regulator | 1728.846 | 1760 | 1404.955 | 1630.486 | −2.99E−01 | 2.42E−02 | 2 | Unchanged Medium | |
| P41236 | C08cd1 | protein phosphatase 1; regulator | 532.1136 | 621 | 583.7663 | 578.9436 | 1.34E−01 | 2.23E−01 | 2 | Unchanged Medium | |
| Q15435 | D15ab8 | protein phosphatase 1; regulator | 634.9759 | 963 | 753.9046 | 784.0242 | 2.48E−01 | 6.01E−01 | 2 | Unchanged Medium | |
| P05323 | E13ef6 | protein phosphatase 2 (formerly: | 1058.276 | 1120 | 796.6526 | 992.192 | −4.10E−01 | 8.39E−02 | 2 | Unchanged Medium | |
| Q15257 | G01ef5 | protein phosphatase 2A; regulat | 925.4458 | 905 | 1160.437 | 996.8116 | 3.26E−01 | −3.29E−02 | 2 | Unchanged Medium | |
| P33172 | F01ab8 | protein phosphatase 4 (formerly | 1791.171 | 1220 | 1064.665 | 1357.256 | −7.S1E−01 | −5.59E−01 | 2 | Unchanged Medium | |
| O00743 | F03ab8 | protein phosphatasa 6; catalytic | 998.7 | 1070 | 738.4815 | 936.2693 | −4.35E−01 | 1.02E−01 | 2 | Unchanged Medium | |
| P38391 | E21cd7 | protein translocation complex be | 1049.306 | 1050 | 1428.451 | 1175.447 | 4.45E−01 | −9.93E−04 | 2 | Unchanged Medium | |
| P78324 | M17cd4 | protein tyrosine phosphatase; no | 639.2381 | 741 | 591.4877 | 657.1953 | −1.12E−01 | 2.13E−01 | 2 | Unchanged Medium | |
| Q13332 | P21ab8 | protein tyrosine phosphatase; re | 1882.284 | 1880 | 2019.865 | 1928.827 | 1.02E−01 | 1.57E−03 | 2 | Unchanged Medium | |
| Q9HAD6 | C08gh6 | protocadherin 16 dachsous-like ( | 967.2552 | 906 | 1070.494 | 981.1143 | 1.46E−01 | −9.50E−02 | 2 | Unchanged Medium | |
| O43445 | E11cd5 | PRP4 pre-mRNA processing fact | 777.1081 | 549 | 569.3749 | 631.8368 | −4.49E−01 | −5.01E−01 | 2 | Unchanged Medium | |
| Q9Y6B3 | L19ef2 | PTD013 protein | 652.1251 | 701 | 1031.739 | 794.9108 | 6.62E−01 | 1.04E−01 | 2 | Unchanged Medium | |
| Q9HAN1 | P19gh6 | pumuilio homolog 1 (Drosophila) | 842.4925 | 1170 | 1047.18 | 1019.233 | 3.14E−01 | 4.71E−01 | 2 | Unchanged Medium | |
| Q9NQA4 | C23ef4 | putative acid-sensing ion channe | 742.723 | 762 | 678.3813 | 727.7123 | −1.31E−01 | 3.70E−02 | 2 | Unchanged Medium | |
| O43598 | J05cd6 | putative c-Myc-responsive | 473.6498 | 621 | 459.383 | 517.9666 | −4.416−02 | 3.90E−01 | 2 | Unchanged Medium | |
| O43257 | M12cd6 | putative cyctin G1 interacting pro | 1118.374 | 1190 | 1238.925 | 1183.655 | 1.48E−01 | 9.40E−02 | 2 | Unchanged Medium | |
| Q9NY06 | M07ef4 | putative integral membrane trans | 966.4384 | 544 | 569.8565 | 693.4947 | −7.62E−01 | −8.29E−01 | 2 | Unchanged Medium | |
| P17152 | H21cd4 | putative receptor protein | 399.9305 | 650 | 591.3162 | 547.029 | 5.64E−01 | 7.00E−01 | 2 | Unchanged Medium | |
| Q9Y619 | B13ef2 | putative secreted protein | 373.307 | 485 | 742.537 | 533.7168 | 9.92E−01 | 3.79E−01 | 2 | Unchanged Medium | |
| P54886 | B12ab8 | pyrroline-5-carboxylate synthetas | 406.7128 | 302 | 905.8993 | 538.0842 | 1.16E+00 | −4.31E−01 | 2 | Unchanged Medium | |
| Q13876 | B03ef7 | guiescin Q6 | 745.299 | 585 | 824.2489 | 718.0657 | 1.45E−01 | −3.50E−01 | 2 | Unchanged Medium | |
| O14679 | K24ef5 | quinone oxidoreductase homolog | 710.5753 | 1160 | 651.2257 | 839.1208 | −1.26E−01 | 7.02E−01 | 2 | Unchanged Medium | |
| P53611 | N16ab8 | Rab geranylgeranyltransferase: b | 758.1496 | 727 | 938.6179 | 807.7755 | 3.08E−01 | −6.14E−02 | 2 | Unchanged Medium | |
| P47224 | D06ab8 | RAB interacting factor | 645.5902 | 859 | 577.3956 | 693.8748 | −1.61E−01 | 4.11E−01 | 2 | Unchanged Medium | |
| Q15907 | H04cd4 | RAB11B; member RAS oncogene | 926.8757 | 1030 | 1224.758 | 1060.693 | 4.02E−01 | 1.53E−01 | 2 | Unchanged Medium | |
| P51153 | D04ab8 | RAB13; member RAS oncogene | 619.0867 | 935 | 1131.86 | 695.2783 | 8.70E−01 | 5.95E−01 | 2 | Unchanged Medium | |
| P35287 | J24ef2 | RAB14; member RAS oncogene | 543.7425 | 584 | 586.1635 | 571.2023 | 1.08E−01 | 1.02E−01 | 2 | Unchanged Medium | |
| Q9NP72 | P02ef8 | RAB18; member RAS oncogene | 459.4786 | 794 | 634.6616 | 629.3713 | 4.66E−01 | 7.89E−01 | 2 | Unchanged Medium | |
| P11476 | H22ab8 | RAB1A; member RAS oncogene | 1039.455 | 767 | 993.586 | 933.4801 | −6.516−02 | −4.38E−01 | 2 | Unchanged Medium | |
| Q9H0U4 | L08gh7 | RAB1B; member RAS oncogene | 526.9882 | 691 | 696.5292 | 638.0939 | 4.02E−01 | 3.90E−01 | 2 | Unchanged Medium | |
| P08886 | B22ab8 | RAB2; member RAS oncogene fa | 1483.703 | 1900 | 2195.304 | 1858.384 | 5.65E−01 | 3.54E−01 | 2 | Unchanged Medium | |
| O95716 | P04cd4 | RAB3D; member RAS oncogene | 908.9015 | 638 | 556.2242 | 701.181 | −7.08E−01 | −5.10E−01 | 2 | Unchanged Medium | |
| P35239 | D02ab8 | RAB5B; member RAS oncogene | 598.5113 | 708 | 393.3892 | 566.5496 | −6.05E−01 | 2.42E−01 | 2 | Unchanged Medium | |
| P20340 | C07ef6 | RAB6A; member RAS oncogene | 503.1507 | 655 | 416.7552 | 524.9786 | −2.72E−01 | 3.81E−01 | 2 | Unchanged Medium | |
| P43487 | D16ab8 | RAN binding protein 1 | 1438.305 | 1180 | 1766.177 | 1468.585 | 3.13E−01 | −2.84E−01 | 2 | Unchanged Medium | |
| Q9UI26 | B20ef1 | Ran binding protein 11 | 1505.224 | 1180 | 1273.677 | 1319.368 | −2.41E−01 | −3.52E−01 | 2 | Unchanged Medium | |
| P47736 | D18ab8 | RAP1; GTPase activating protein | 875.8692 | 414 | 461.3322 | 583.6391 | −9.25E−01 | −1.08E+00 | 2 | Unchanged Medium | |
| Q15382 | F17cd2 | Ras homolog enriched in brain 2 | 1435.587 | 2190 | 1635.792 | 1754.23 | 1.88E−01 | 6.10E−01 | 2 | Unchanged Medium | |
| P52199 | B18ab2 | ras homolog gene family: membe | 534.6169 | 1520 | 399.0517 | 816.6152 | −4.22E−01 | 1.50E+00 | 2 | Unchanged Medium | |
| P35238 | B20ab2 | ras homolog gene family: membe | 1022.646 | 872 | 1019.472 | 971.4616 | −4.486−03 | −2.29E−01 | 2 | Unchanged Medium | |
| Q14644 | J08ef7 | RAS p21 protein activator (GTPa | 615.946 | 623 | 627.059 | 622.1475 | 2.586−02 | 1.74E−02 | 2 | Unchanged Medium | |
| Q15404 | A10cd8 | Ras suppressor protein 1 | 1543.953 | 1920 | 2317.155 | 1925.544 | 5.86E−01 | 3.11E−01 | 2 | Unchanged Medium | |
| Q13283 | H14cd5 | Ras-GTPase-activating protein S | 846.7267 | 789 | 571.1476 | 735.5498 | −5.68E−01 | −1.02E−01 | 2 | Unchanged Medium | |
| Q9NVQ9 | M05gh1 | Rec8p: a meiotic recombination | 1334.371 | 1200 | 1325.85 | 1286.027 | −9.246−03 | −1.56E−01 | 2 | Unchanged Medium | |
| O60895 | K17cd6 | receptor (calcitonin) activity modi | 572.8174 | 519 | 899.2606 | 663.6562 | 6.51E−01 | −1.43E−01 | 2 | Unchanged Medium | |
| P35244 | I22ef6 | replication protein A3 (14kD) | 544.0212 | 502 | 924.5385 | 656.7974 | 7.65E−01 | −1.16E−01 | 2 | Unchanged Medium | |
| Q99623 | D03cd7 | repressor of estrogen receptor a | 1139.283 | 689 | 1233.151 | 1020.486 | 1.14E−01 | −7.25E−01 | 2 | Unchanged Medium | |
| Q9UMQ4 | C01gh1 | ret proto-oncogene (multiple end | 769.2484 | 602 | 803.931 | 725.1999 | 6.36E−02 | −3.53E−01 | 2 | Unchanged Medium | |
| Q9NQC3 | M19cd7 | reticulon 4 | 1459.586 | 586 | 927.9776 | 991.1835 | −6.53E−01 | −1.32E+00 | 2 | Unchanged Medium | |
| P47804 | P03cd1 | retinal G protein coupled recepto | 403.1219 | 497 | 627.4176 | 509.1489 | 6.38E−01 | 3.02E−01 | 2 | Unchanged Medium | |
| Q16576 | P07ef6 | retinoblastoma binding protein 7 | 1539.008 | 1410 | 798.8744 | 1250.347 | −9.46E−01 | −1.23E−01 | 2 | Unchanged Medium | |
| O95357 | D02cd4 | retinoic acid induced 3 | 678.1926 | 713 | 206.6352 | 532.4837 | −1.71E+00 | 7.14E−02 | 2 | Unchanged Medium | |
| Q15299 | D20ef1 | retinoic acid receptor-beta assoc | 1060.232 | 979 | 1013.444 | 1017.803 | −6.51E−02 | −1.15E−01 | 2 | Unchanged Medium | |
| Q9UMY1 | P05ef2 | retinoic acid repressible protein | 610.5279 | 726 | 586.2413 | 641.044 | −5.86E−02 | 2.51E−01 | 2 | Unchanged Medium | |
| Q13017 | M15ef6 | Rho GTPase activating protein 5 | 560.443 | 807 | 514.6138 | 627.5049 | −1.23E−01 | 5.27E−01 | 2 | Unchanged Medium | |
| O43182 | B24ab2 | Rho GTPase activating protein 6 | 1257.146 | 806 | 895.7191 | 986.3979 | −4.89E−01 | −6.41E−01 | 2 | Unchanged Medium | |
| O60274 | F04gh6 | Rho-specific guanine nucleotide | 651.9484 | 577 | 366.4761 | 531.8448 | −8.31E−01 | −1.76E−01 | 2 | Unchanged Medium | |
| P07998 | P21cd1 | ribonuclease: RNase A family: 1 | 1294.476 | 3040 | 1592.811 | 1976.991 | 2.99E−01 | 1.23E+00 | 2 | Unchanged Medium | |
| P04843 | B12cd1 | ribophorin I | 1493.814 | 1360 | 2248.214 | 1700.316 | 5.90E−01 | −1.37E−01 | 2 | Unchanged Medium | |
| P04844 | B14cd1 | ribophorin II | 1574.181 | 1170 | 1880.047 | 1540.461 | 2.56E−01 | −4.32E−01 | 2 | Unchanged Medium | |
| P26373 | D11cd1 | ribosomal protein L13 | 2091.256 | 1200 | 1924.546 | 1739.922 | −1.20E−01 | −7.97E−01 | 2 | Unchanged Medium | |
| P40429 | H12ef1 | ribosomal protein L13a | 1928.024 | 2060 | 1091.46 | 1694.794 | −8.21E−01 | 9.89E−02 | 2 | Unchanged Medium | |
| P39019 | J05cd1 | ribosomal protein S19 | 1794.644 | 1480 | 2253.771 | 1841.785 | 3.29E−01 | −2.81E−01 | 2 | Unchanged Medium | |
| P78317 | B04cd1 | ring finger protein 4 | 756.2395 | 661 | 668.8027 | 695.4611 | −1.77E−01 | −1.93E−01 | 2 | Unchanged Medium | |
| Q99942 | F04cd2 | ring finger protein 5 | 1018.837 | 949 | 1126.187 | 1030.595 | 1.47E−01 | −1.00E−01 | 2 | Unchanged Medium | |
| O43148 | A15cd4 | RNA (guanine-7-) methyltransfer | 572.8495 | 730 | 390.498 | 564.3647 | −5.52E−01 | 3.50E−01 | 2 | Unchanged Medium | |
| O02916 | F06ab8 | RNA binding motif protein 4 | 533.7121 | 628 | 430.5298 | 530.87 | −3.10E−01 | 2.36E−01 | 2 | Unchanged Medium | |
| Q9Y580 | J10gh6 | RNA binding motif protein 7 | 368.8423 | 710 | 558.043 | 545.5838 | 5.97E−01 | 9.45E−01 | 2 | Unchanged Medium | |
| Q14621 | H07cd7 | RNA binding protein (autoantige | 499.5699 | 737 | 506.822 | 581.142 | 2.08E−02 | 5.61E−01 | 2 | Unchanged Medium | |
| Q93062 | K17cd7 | RNA-binding protein gene with | 845.0994 | 659 | 640.4514 | 714.6837 | −4.00E−01 | −3.60E−01 | 2 | Unchanged Medium | |
| Q99497 | B15cd1 | RNA-binding protein regulatory s | 1914.86 | 1400 | 2563.974 | 1958.111 | 4.21E−01 | −4.56E−01 | 2 | Unchanged Medium | |
| Q99584 | L17cd2 | S100 calcium binding protein A1: | 492.6196 | 421 | 675.1457 | 529.713 | 4.55E−01 | −2.25E−01 | 2 | Unchanged Medium | |
| P23526 | A06ef1 | S-adenosylhomocysteine hydrola | 550.6181 | 603 | 478.692 | 544.258 | −2.02E−01 | 1.32E−01 | 2 | Unchanged Medium | |
| O43865 | N21ab2 | S-adenosylhomocysteine hydrola | 1460.581 | 526 | 486.4887 | 817.8431 | −1.85E+00 | −1.47E+00 | 2 | Unchanged Medium | |
| O14828 | D02cd5 | secretory carrier membrane prote | 661.3975 | 460 | 481.9262 | 534.3923 | −4.57E−01 | −5.24E−01 | 2 | Unchanged Medium | |
| O75326 | L05cd3 | sema domain: immunoglobulin d | 795.0564 | 768 | 943.1132 | 835.2276 | 2.46E−01 | −5.09E−02 | 2 | Unchanged Medium | |
| O43278 | I16cd2 | serine protease inhibitor; Kunitz | 1295.263 | 1280 | 641.0775 | 1086.162 | −1.01E+00 | −3.74E−02 | 2 | Unchanged Medium | |
| O00271 | C05ef5 | serine protease inhibitor: Kunitz | 1379.545 | 1740 | 876.4128 | 1332.552 | −6.55E−01 | 3.36E−01 | 2 | Unchanged Medium | |
| Q9UEW8 | N22ef5 | serine threonine kinase 39 (STE | 1230.117 | 1030 | 841.1872 | 1032.185 | −5.48E−01 | −2.63E−01 | 2 | Unchanged Medium | |
| Q13043 | P15cd2 | serine/threonine kinase 4 | 750.7132 | 838 | 756.1662 | 781.5546 | 1.04E−02 | 1.58E−01 | 2 | Unchanged Medium | |
| Q9Y282 | F01ef2 | serologically defined breast canc | 1279.414 | 1160 | 1907.544 | 1448.986 | 5.76E−01 | −1.41E−01 | 2 | Unchanged Medium | |
| P35542 | A02gh1 | serum amyloid A4; constitutive | 754.5041 | 1310 | 811.5701 | 959.3499 | 1.05E−01 | 7.98E−01 | 2 | Unchanged Medium | |
| O75790 | I22cd6 | seven transmembrane domain p | 761.3872 | 778 | 799.0998 | 779.5745 | 6.97E−02 | 3.16E−02 | 2 | Unchanged Medium | |
| Q9P0V3 | H17ef3 | SH3-domain binding protein 4 | 1204.005 | 1710 | 1539.363 | 1483.624 | 3.54E−01 | 5.04E−01 | 2 | Unchanged Medium | |
| Q99519 | C13ab8 | sialidase 1 (lysosomal sialidase) | 712.4593 | 680 | 498.7449 | 630.4428 | −5.15E−01 | −6.70E−02 | 2 | Unchanged Medium | |
| Q92185 | J20cd1 | sialyltransferase 8A (alpha-N-ac | 622.4236 | 478 | 462.7941 | 520.9105 | −4.28E−01 | −3.82E−01 | 2 | Unchanged Medium | |
| Q9Y6A9 | C05ef8 | signal peptidase 12kDa | 449.125 | 742 | 887.4615 | 692.8575 | 9.83E−01 | 7.24E−01 | 2 | Unchanged Medium | |
| P21378 | I04ef3 | signal peptidase complex (18kD) | 630.5617 | 515 | 732.1592 | 626.0198 | 2.16E−01 | −2.91E−01 | 2 | Unchanged Medium | |
| O15302 | P10ab3 | signal recognition particle 72kD | 1040.23 | 818 | 841.5078 | 899.9534 | −3.06E−01 | −3.47E−01 | 2 | Unchanged Medium | |
| O76094 | J02cd2 | signal recognition particle 72kD | 1473.58 | 965 | 1422.2 | 1286.822 | −5.12E−02 | −6.11E−01 | 2 | Unchanged Medium | |
| Q9UNL2 | J10cd2 | signal sequence receptor: gamm | 1150.724 | 450 | 1654.744 | 1085.128 | 5.24E−01 | −1.35E+00 | 2 | Unchanged Medium | |
| P40763 | K09ef5 | signal transducer and activator o | 671.5038 | 545 | 348.0347 | 521.4351 | −9.48E−01 | −3.02E−01 | 2 | Unchanged Medium | |
| Q92783 | M24cd3 | signal transducing adaptor molec | 827.9851 | 1020 | 725.7017 | 857.8322 | −1.90E−01 | 3.01E−01 | 2 | Unchanged Medium | |
| Q9H723 | N01gh5 | similar to rat nuclear ubiquitous | 749.7661 | 530 | 1258.65 | 846.2392 | 7.47E−01 | −5.00E−01 | 2 | Unchanged Medium | |
| O15258 | A04cd7 | similar to S. cerevisiae RER1 | 710.8499 | 635 | 933.6382 | 759.8427 | 3.93E−01 | −1.63E−01 | 2 | Unchanged Medium | |
| O14834 | A11cd7 | similar to S. pombe dim1+ | 701.2292 | 753 | 903.8926 | 786.0877 | 3.66E−01 | 1.03E−01 | 2 | Unchanged Medium | |
| O43617 | H05ef3 | similar to yeast BET3 (S. cerevisiae) | 1759.348 | 1750 | 1443.578 | 1650.871 | −2.85E−01 | −7.94E−03 | 2 | Unchanged Medium | |
| Q04837 | G11cd2 | single-stranded DNA binding pro | 1284.277 | 894 | 1034.75 | 1084.292 | −2.89E−01 | −5.00E−01 | 2 | Unchanged Medium | |
| P05455 | E19gh1 | Sjogren syndrome antigen B (aut | 850.9691 | 744 | 930.6428 | 841.9268 | 1.29E−01 | −1.93E−01 | 2 | Unchanged Medium | |
| P43331 | K08cd2 | small nuclear ribonucleoprotein | 916.3597 | 1280 | 1042.335 | 1073.82 | 1.86E−01 | 4.63E−01 | 2 | Unchanged Medium | |
| P09012 | M12cd2 | small nuclear ribonucleoprotein | 548.0261 | 310 | 685.6636 | 514.5708 | 3.23E−01 | −8.22E−01 | 2 | Unchanged Medium | |
| P08579 | A03cd2 | small nuclear ribonucleoprotein | 502.6355 | 570 | 669.9879 | 581.0279 | 4.15E−01 | 1.83E−01 | 2 | Unchanged Medium | |
| P09234 | A05cd2 | small nuclear ribonucleoprotein | 1054.327 | 1450 | 1302.742 | 1269.84 | 3.05E−01 | 4.62E−01 | 2 | Unchanged Medium | |
| P08578 | A07cd2 | small nuclear ribonucleoprotein | 1901.214 | 1740 | 1818.557 | 1819.876 | −6.41E−02 | −1.28E−01 | 2 | Unchanged Medium | |
| Q15357 | A11cd2 | small nuclear ribonucleoprotein | 382.4892 | 734 | 593.847 | 570.2015 | 6.35E−01 | 9.41E−01 | 2 | Unchanged Medium | |
| P14678 | C23cd3 | small nuclear ribonucleoprotein | 2036.574 | 1280 | 1737.398 | 1686.175 | −2.29E−01 | −6.65E−01 | 2 | Unchanged Medium | |
| Q13487 | P08cd1 | small nuclear RNA activating co | 517.5819 | 684 | 648.0121 | 616.6789 | 3.24E−01 | 4.03E−01 | 2 | Unchanged Medium | |
| P35326 | E17cd3 | small proline-rich protein 2A | 290.8174 | 1030 | 839.3072 | 721.2318 | 1.53E+00 | 1.83E+00 | 2 | Unchanged Medium | |
| P55854 | H04cd2 | SMT3 suppressor of mif two 3 ho | 791.7437 | 658 | 962.4453 | 804.107 | 2.82E−01 | −2.67E−01 | 2 | Unchanged Medium | |
| P14848 | P19cd5 | SNRPN upstream reading frame | 1581.907 | 2270 | 1670.964 | 1839.36 | 7.90E−02 | 5.18E−01 | 2 | Unchanged Medium | |
| P55011 | L17cd1 | solute carrier family 12 (sodium/p | 591.8878 | 740 | 517.2382 | 616.497 | −1.94E−01 | 3.23E−01 | 2 | Unchanged Medium | |
| O95258 | P15cd4 | solute carrier family 25 (mitocho | 476.3075 | 518 | 577.8319 | 523.9535 | 2.79E−01 | 1.20E−01 | 2 | Unchanged Medium | |
| O15431 | A21ab5 | solute carrier family 31 (copper | 1227.872 | 1010 | 1769.026 | 1336.855 | 5.27E−01 | −2.77E−01 | 2 | Unchanged Medium | |
| P04920 | L04cd1 | solute carrier family 4; anion excl | 1614.362 | 1560 | 1704.134 | 1624.765 | 7.81E−02 | −5.33E−02 | 2 | Unchanged Medium | |
| Q01650 | J11ef5 | solute carrier family 7 (cationic a | 832.6996 | 632 | 1032.106 | 832.1088 | 3.10E−01 | −3.99E−01 | 2 | Unchanged Medium | |
| Q07890 | H23cd2 | son of sevenless homolog 2 (Dro | 476.9505 | 568 | 680.8529 | 575.1528 | 5.14E−01 | 2.51E−01 | 2 | Unchanged Medium | |
| O60749 | A19cd2 | sorting nexin 2 | 384.9343 | 931 | 769.5782 | 688.5915 | 1.08E+00 | 1.35E+00 | 2 | Unchanged Medium | |
| O60493 | A09cd4 | sorting nexin 3 | 1008.916 | 1350 | 1180.469 | 1180.399 | 2.27E−01 | 4.22E−01 | 2 | Unchanged Medium | |
| Q9Y5X3 | G06ef4 | sorting nexin 5 | 676.0975 | 647 | 688.5568 | 670.591 | 2.63E−02 | −6.32E−02 | 2 | Unchanged Medium | |
| Q01082 | E09cd2 | spectrin: beta: non-erythrocytic 1 | 571.4909 | 679 | 387.0403 | 545.8922 | −5.62E−01 | 2.49E−01 | 2 | Unchanged Medium | |
| P52788 | M10cd2 | spermine synthase | 895.1994 | 854 | 770.002 | 839.7693 | −2.17E−01 | −6.78E−02 | 2 | Unchanged Medium | |
| P17947 | C19cd2 | spleen focus forming virus (SFF | 1307.418 | 1150 | 1646.278 | 1366.573 | 3.32E−01 | −1.90E−01 | 2 | Unchanged Medium | |
| Q14818 | D10ef6 | splicing factor 1 | 547.0998 | 499 | 782.2929 | 609.3662 | 5.16E−01 | −1.34E−01 | 2 | Unchanged Medium | |
| Q15427 | K11cd6 | splicing factor 3b: subunit 4: 49kD | 441.2394 | 612 | 540.0723 | 530.9445 | 2.92E−01 | 4.71E−01 | 2 | Unchanged Medium | |
| Q15815 | M06cd2 | splicing factor: arginine/serine-ri | 738.2591 | 553 | 551.9082 | 614.261 | −4.20E−01 | −4.18E−01 | 2 | Unchanged Medium | |
| Q01130 | H22cd1 | splicing factor: arginine/serine-ri | 553.5808 | 527 | 488.5465 | 522.9409 | −1.80E−01 | −7.18E−02 | 2 | Unchanged Medium | |
| P23152 | H24cd1 | splicing factor: arginine/serine-ri | 1547.474 | 1460 | 1428.273 | 1480.13 | −1.16E−01 | −7.94E−02 | 2 | Unchanged Medium | |
| Q13503 | L22cd4 | SRB7 suppressor of RNA polyme | 712.8859 | 574 | 725.0591 | 670.5377 | 2.44E−02 | −3.13E−01 | 2 | Unchanged Medium | |
| Q9P2R9 | C23ef2 | SRp25 nuclear protein | 495.796 | 558 | 515.095 | 522.8318 | 5.51E−02 | 1.69E−01 | 2 | Unchanged Medium | |
| Q9H612 | F07gh5 | SRY (sex determining region Y)-I | 774.5162 | 970 | 714.8666 | 819.674 | −1.16E−01 | 3.24E−01 | 2 | Unchanged Medium | |
| Q9BT81 | I13gh8 | SRY (sex determining region Y)-I | 497.0608 | 530 | 550.1706 | 525.7297 | 1.46E−01 | 9.25E−02 | 2 | Unchanged Medium | |
| O60526 | M03cd6 | STIP 1 homology and U-Box cont | 706.6078 | 538 | 543.7559 | 596.1019 | −3.78E−01 | −3.93E−01 | 2 | Unchanged Medium | |
| Q9Y6X1 | A21ef4 | stress-associated endoplasmic r | 1683.36 | 1220 | 2201.647 | 1700.638 | 3.87E−01 | −4.68E−01 | 2 | Unchanged Medium | |
| P31948 | M15ef3 | stress-induced-phosphoprotein 1 | 436.1143 | 549 | 517.5834 | 500.9139 | 2.47E−01 | 3.32E−01 | 2 | Unchanged Medium | |
| P21912 | E09gh1 | succinate dehydrogenase compl | 701.7228 | 902 | 751.7357 | 785.3111 | 9.93E−02 | 3.63E−01 | 2 | Unchanged Medium | |
| O14521 | H04cd1 | succinate dehydrogenase compL | 628.8405 | 505 | 672.375 | 601.9355 | 9.66E−02 | 3.18E−01 | 2 | Unchanged Medium | |
| P53597 | I09gh1 | succinate-CoA ligase: GDP-form | 704.4006 | 491 | 553.293 | 582.7447 | −3.48E−01 | −5.22E−01 | 2 | Unchanged Medium | |
| O95605 | P15cd5 | SUMO-1 activating enzyme subu | 776.4199 | 499 | 433.2742 | 569.4163 | −8.42E−01 | −6.39E−01 | 2 | Unchanged Medium | |
| Q16550 | K01cd2 | suppressor of Ty 4 homolog 1 (S | 813.5735 | 1440 | 991.3588 | 1081.744 | 2.85E−01 | 8.24E−01 | 2 | Unchanged Medium | |
| Q15526 | K07cd2 | surfeit 1 | 468.3614 | 522 | 583.8097 | 524.7026 | 3.18E−01 | 1.56E−01 | 2 | Unchanged Medium | |
| O43539 | P02cd1 | SWI/SNF related; matrix associa | 1047.14 | 1280 | 1212.614 | 1178.519 | 2.12E−01 | 2.85E−01 | 2 | Unchanged Medium | |
| Q9UPX1 | D23cd7 | synaptopodin | 683.4973 | 536 | 421.1841 | 546.8685 | −6.98E−01 | −3.51E−01 | 2 | Unchanged Medium | |
| O95721 | O09cd5 | synaptosomal-associated protein | 830.7721 | 1030 | 686.6755 | 649.1506 | −2.75E−01 | 3.10E−01 | 2 | Unchanged Medium | |
| O43391 | H09cd2 | syndecan binding protein (synter | 625.6962 | 640 | 379.1899 | 548.1569 | −7.23E−01 | 3.17E−02 | 2 | Unchanged Medium | |
| Q15833 | J04cd2 | syntaxin binding protein 2 | 661.0175 | 556 | 840.6951 | 686.0183 | 3.47E−01 | −2.49E−01 | 2 | Unchanged Medium | |
| Q92804 | O20cd3 | TAF15 RNA polymerase II: TATA | 873.5564 | 605 | 1063.778 | 847.4936 | 2.84E−01 | −5.30E−01 | 2 | Unchanged Medium | |
| Q9Y490 | B04cd2 | talin 1 | 1342.238 | 1170 | 2365.625 | 1625.187 | 8.18E−01 | −2.01E−01 | 2 | Unchanged Medium | |
| O94797 | H20ef7 | Tara-like protein | 899.0807 | 448 | 496.1404 | 614.3777 | −8.58E−01 | −1.01E+00 | 2 | Unchanged Medium | |
| Q13311 | C05cd6 | Tax1 (human T-cell leukemia viru | 899.9979 | 1190 | 926.2314 | 1004.505 | 4.15E−02 | 4.00E−01 | 2 | Unchanged Medium | |
| Q16650 | F08cd6 | T-box: brain; 1 | 1407.309 | 1750 | 1777.889 | 1645.184 | 3.37E−01 | 3.15E−01 | 2 | Unchanged Medium | |
| P51864 | O04ef7 | teratocarcinoma-derived growth | 2037.212 | 1590 | 1447.46 | 1690.282 | −4.93E−01 | −3.61E−01 | 2 | Unchanged Medium | |
| P55061 | O19cd2 | testis enhanced gene transcript ( | 1540.968 | 1330 | 1972.323 | 1615.143 | 3.56E−01 | −2.10E−01 | 2 | Unchanged Medium | |
| Q15569 | P21cd2 | testis-specific kinase 1 | 837.6269 | 796 | 708.1178 | 780.661 | −2.42E−01 | −7.31E−02 | 2 | Unchanged Medium | |
| O60636 | F08cd5 | tetraspan 2 | 468.1664 | 702 | 375.3225 | 515.2157 | −3.19E−01 | 5.85E−01 | 2 | Unchanged Medium | |
| O60637 | F06cd5 | tetraspan 3 | 1025.362 | 742 | 545.9662 | 770.9441 | −9.09E−01 | −4.68E−01 | 2 | Unchanged Medium | |
| O95857 | F13ef3 | tetraspan NET-6 protein | 702.1579 | 676 | 359.1921 | 578.9636 | −9.67E−01 | −5.58E−02 | 2 | Unchanged Medium | |
| Q99614 | M03cd3 | tetratricopeptide repeat domain 1 | 706.3536 | 891 | 635.5379 | 744.2616 | −1.52E−01 | 3.35E−01 | 2 | Unchanged Medium | |
| Q13118 | L05cd2 | TGFB inducible early growth res | 445.9691 | 601 | 679.2931 | 575.4732 | 6.07E−01 | 4.31E−0l | 2 | Unchanged Medium | |
| Q15563 | A16cd2 | TGFB-induced factor (TALE fami | 885.9 | 1290 | 1140.439 | 1105.268 | 3.64E−01 | 5.42E−01 | 2 | Unchanged Medium | |
| Q9H3N1 | N03gh7 | thioredoxin domain-containing | 409.6333 | 426 | 686.7997 | 507.428 | 7.46E−01 | 5.60E−02 | 2 | Unchanged Medium | |
| O43396 | O17cd5 | thioredoxin-like: 32kD | 1821.595 | 1710 | 1548.596 | 1693.179 | −2.34E−01 | −9.18E−02 | 2 | Unchanged Medium | |
| P04216 | B02cd2 | Thy-1 cell surface antigen | 169.8064 | 298 | 4157.367 | 1541.609 | 4.61E+00 | 8.10E−01 | 2 | Unchanged Medium | |
| P12956 | K10ef6 | thyroid autoantigen 70kD (Ku an | 1172.56 | 1490 | 1395.803 | 1353.001 | 2.51E−01 | 3.46E−01 | 2 | Unchanged Medium | |
| P16035 | P11ef7 | tissue inhibitor of metalloproteina | 360.2533 | 354 | 913.1351 | 542.4517 | 1.34E+00 | −2.54E−02 | 2 | Unchanged Medium | |
| Q9H2X8 | I08gh8 | TLH29 protein precursor | 710.2447 | 1000 | 1386.102 | 1033.648 | 9.65E−01 | 5.00E−01 | 2 | Unchanged Medium | |
| Q13077 | L11cd2 | TNF receptor-associated factor 1 | 1265.054 | 1270 | 756.401 | 1097.404 | −7.42E−01 | 6.49E−03 | 2 | Unchanged Medium | |
| Q9NZ34 | P08gh4 | TPA regulated locus | 1074.319 | 1090 | 1264.44 | 1148.575 | 2.58E−01 | 1.69E−02 | 2 | Unchanged Medium | |
| Q15369 | D18ef6 | transcription elongation factor B | 771.5598 | 1100 | 693.1767 | 854.8396 | −1.55E−01 | 5.11E−01 | 2 | Unchanged Medium | |
| Q15906 | P11ef6 | transcription factor-like 1 | 436.5561 | 510 | 878.9083 | 608.5936 | 1.01E+00 | 2.25E−01 | 2 | Unchanged Medium | |
| P50616 | H08cd5 | tranducer of ERBB2; 1 | 888.8059 | 874 | 518.1816 | 760.3803 | −7.78E−01 | −2.40E−02 | 2 | Unchanged Medium | |
| Q13595 | N04ef7 | transformer-2 alpha (htra-2 alpha | 247.4437 | 612 | 698.379 | 519.219 | 1.50E+00 | 1.31E+00 | 2 | Unchanged Medium | |
| P37802 | F05cd3 | transgelin 2 | 1986.716 | 2240 | 1173.434 | 1798.473 | −7.60E−01 | 1.70E−01 | 2 | Unchanged Medium | |
| Q9Y5Z9 | D01cd8 | transitional epithelia response pr | 1324.284 | 875 | 1126.793 | 1108.824 | −2.33E−01 | −5.97E−01 | 2 | Unchanged Medium | |
| Q99595 | K10cd6 | translocase of inner mitochondria | 450.6578 | 665 | 448.5161 | 521.2602 | −6.87E−03 | 5.60E−01 | 2 | Unchanged Medium | |
| O60830 | A09cd6 | translocase of inner mitochondria | 1646.564 | 1590 | 1992.925 | 1744.175 | 2.75E−01 | −4.77E−02 | 2 | Unchanged Medium | |
| O14925 | I24cd6 | translocase of inner mitochondria | 745.8102 | 813 | 653.4301 | 737.451 | −1.91E−01 | 1.25E−01 | 2 | Unchanged Medium | |
| Q9Y5J9 | E08cd8 | translocase of inner mitochondria | 1641.377 | 1550 | 1653.524 | 1615.991 | 1.06E−02 | −7.98E−02 | 2 | Unchanged Medium | |
| Q15629 | G18ef3 | translocating chain-associating | 1003.031 | 1060 | 624.4499 | 897.0752 | −6.84E−01 | 8.48E−02 | 2 | Unchanged Medium | |
| Q99442 | C10cd2 | translocation protein 1 | 526.8783 | 772 | 1065.397 | 788.001 | 1.02E+00 | 5.51E−01 | 2 | Unchanged Medium | |
| Q99805 | A18cd5 | transmembrane 9 superfamily m | 1023.368 | 1260 | 957.2801 | 1081.028 | −9.63E−02 | 3.03E−01 | 2 | Unchanged Medium | |
| Q9Y2B0 | C20ef3 | transmembrane protein 4 | 681.3857 | 1320 | 985.8397 | 995.6424 | 5.33E−01 | 9.54E−01 | 2 | Unchanged Medium | |
| P49755 | J05ef4 | transmembrane trafficking protein | 1311.203 | 469 | 700.7243 | 827.066 | −9.04E−01 | −1.46E+00 | 2 | Unchanged Medium | |
| | F03gh4 | transmembrane: prostate androg | 1287.98 | 841 | 662.4165 | 930.4505 | −9.59E−01 | −6.15E−01 | 2 | Unchanged Medium | |
| Q03519 | B07cd1 | transporter 2; ATP-binding casse | 851.2148 | 573 | 680.8519 | 701.534 | −3.22E−01 | −5.72E−01 | 2 | Unchanged Medium | |
| Q9NWF9 | P02gh2 | TRIAD3 protein | 665.9772 | 444 | 688.6646 | 599.4053 | 4.83E−02 | −5.86E−01 | 2 | Unchanged Medium | |
| Q15656 | O07ef7 | TRK-fused gene | 1878.254 | 1640 | 1858.307 | 1792.501 | −1.54E−02 | −1.95E−01 | 2 | Unchanged Medium | |
| Q92734 | J17cd7 | TRK-fused gene | 1450.307 | 1810 | 1609.067 | 1624.429 | 1.50E−01 | 3.23E−01 | 2 | Unchanged Medium | |
| Q12815 | A21ef7 | trophinin associated protein (last | 821.5601 | 1100 | 722.8644 | 879.96 | −1.85E−01 | 4.15E−01 | 2 | Unchanged Medium | |
| P09493 | O02cd1 | tropomyosin 1 (alpha) | 988.1694 | 429 | 1004.982 | 807.373 | 2.43E−02 | −1.20E+00 | 2 | Unchanged Medium | |
| P49411 | M11cd3 | Tu translation elongation factor | 525.8572 | 555 | 476.4592 | 519.255 | −1.42E−01 | 7.90E−02 | 2 | Unchanged Medium | |
| P05217 | F03gh6 | tubulin: beta; 2 | 1203.686 | 705 | 920.1831 | 942.8088 | −3.87E−01 | −7.73E−01 | 2 | Unchanged Medium | |
| P23258 | K03cd3 | tubulin; gamma 1 | 1733.971 | 1220 | 2149.365 | 1702.331 | 3.10E−01 | −5.03E−01 | 2 | Unchanged Medium | |
| Q9UBN6 | A16ef6 | tumor necrosis factor receptor su | 470.7412 | 751 | 531.524 | 564.4772 | 1.75E−01 | 6.74E−01 | 2 | Unchanged Medium | |
| Q13829 | L22ef4 | tumor necrosis factor; alpha-indu | 849.276 | 792 | 552.7776 | 731.2933 | −6.20E−01 | −1.01E−01 | 2 | Unchanged Medium | |
| O00496 | K21cd3 | tumor suppressing subtransferab | 628.3509 | 554 | 800.8751 | 661.0848 | 3.50E−01 | −1.82E−01 | 2 | Unchanged Medium | |
| Q99816 | G04cd4 | tumor susceptibility gene 101 | 723.8133 | 766 | 627.1295 | 705.4904 | −2.07E−01 | 8.08E−02 | 2 | Unchanged Medium | |
| P16422 | C03ab7 | tumor-associated calcium signal | 1448.512 | 791 | 373.663 | 870.9367 | −1.95E+00 | −8.73E−01 | 2 | Unchanged Medium | |
| Q9NP84 | K03ef2 | type I transmembrane protein Fn | 691.89 | 498 | 560.2824 | 583.2792 | −3.04E−01 | −4.75E−01 | 2 | Unchanged Medium | |
| Q9UJ47 | O09cd8 | type I transmembrane receptor (s | 792.9873 | 819 | 809.9056 | 807.3703 | 3.05E−02 | 4.69E−02 | 2 | Unchanged Medium | |
| P31946 | K21ef6 | tyrosine 3-monooxygenase/trypto | 1220.989 | 955 | 724.9898 | 966.9847 | −7.52E−01 | −3.S5E−01 | 2 | Unchanged Medium | |
| P35214 | J19ef4 | tyrosine 3-monooxygenase/trypto | 881.0131 | 989 | 682.5327 | 850.9964 | −3.68E−01 | 1.67E−01 | 2 | Unchanged Medium | |
| P27348 | G15cd7 | tyrosine 3-monooxygenase/trypto | 2196.334 | 1540 | 1104.855 | 1615.078 | −9.91E−01 | −5.08E−01 | 2 | Unchanged Medium | |
| O60704 | J01cd3 | tyrosylprotein sulfotransferase 2 | 658.3761 | 299 | 686.1339 | 547.7036 | 5.96E−02 | −1.14E+00 | 2 | Unchanged Medium | |
| Q9Y4Z0 | G09cd8 | U6 snRNA-associated Sm-like pr | 837.5087 | 689 | 914.9547 | 813.8478 | 1.28E−01 | −2.81E−01 | 2 | Unchanged Medium | |
| P14927 | G06cd4 | ubiquinol-cytochrome c reductas | 995.1237 | 817 | 954.3085 | 922.1475 | −6.04E−02 | −2.85E−01 | 2 | Unchanged Medium | |
| Q9UI02 | P18ef7 | ubiquinol-cytochrome c reductas | 953.6884 | 1460 | 1546.568 | 1320.75 | 6.97E−01 | 6.16E−01 | 2 | Unchanged Medium | |
| P31930 | A22cd3 | ubiquinol-cytochrome c reductas | 846.6396 | 849 | 686.9864 | 794.0747 | −3.01E−01 | 3.33E−03 | 2 | Unchanged Medium | |
| P22695 | A24cd3 | ubiquinol-cytochrome c reductas | 468.5936 | 636 | 581.791 | 562.0633 | 3.12E−01 | 4.40E−01 | 2 | Unchanged Medium | |
| P07919 | E08cd4 | ubiquinol-cytochrome c reductas | 1328.532 | 1810 | 1005.378 | 1382.391 | −4.02E−01 | 4.49E−01 | 2 | Unchanged Medium | |
| P47985 | E06cd4 | ubiquinol-cytochrome c reductas | 1519.763 | 1270 | 1127.575 | 1306.086 | −4.31E−01 | −2.58E−01 | 2 | Unchanged Medium | |
| Q9NZ09 | N17ef1 | ubiquitin associated protein | 1588.177 | 1820 | 1392.592 | 1598.619 | −1.90E−01 | 1.93E−01 | 2 | Unchanged Medium | |
| Q9UNP0 | F09gh6 | ubiquitin specific protease 15 | 525.3364 | 492 | 597.7817 | 538.3523 | 1.86E−01 | −9.48E−02 | 2 | Unchanged Medium | |
| Q9UHP3 | L21cd8 | ubiquitin specific protease 25 | 554.0657 | 724 | 561.5817 | 613.0845 | 1.94E−02 | 3.85E−01 | 2 | Unchanged Medium | |
| P22314 | O07cd3 | ubiquitin-activating enzyme E1 ( | 1857.238 | 1640 | 1863.316 | 1788.503 | 4.71E−03 | −1.75E−01 | 2 | Unchanged Medium | |
| Q13404 | O23cd3 | ubiquitin-conjugating enzyme E2 | 1073.414 | 1210 | 1128.962 | 1138.559 | 7.28E−02 | 1.77E−01 | 2 | Unchanged Medium | |
| P51669 | D05cd4 | ubiquitin-conjugating enzyme E2 | 1266.25 | 1300 | 1173.73 | 1247.427 | −1.09E−01 | 4.05E−02 | 2 | Unchanged Medium | |
| P47986 | O11cd3 | ubiquitin-conjugating enzyme E2 | 616.3689 | 520 | 567.7317 | 567.8765 | −1.19E−01 | −2.47E−01 | 2 | Unchanged Medium | |
| O14933 | H14cd4 | ubiquitin-conjugating enzyme E2 | 717.4244 | 893 | 596.6415 | 735.6835 | −2.66E−01 | 3.16E−01 | 2 | Unchanged Medium | |
| Q16781 | D07cd4 | ubiquitin-conjugating enzyme E2 | 923.6537 | 816 | 533.7389 | 757.964 | −7.91E−01 | −1.78E−01 | 2 | Unchanged Medium | |
| Q93068 | A02cd3 | ubiquitin-like 1 (sentrin) | 1376.087 | 1530 | 1294.307 | 1399.45 | −8.84E−02 | 1.51E−01 | 2 | Unchanged Medium | |
| Q15386 | I10gh1 | ubiquitin-protein isopeptide ligas | 531.8659 | 630 | 628.2454 | 596.5886 | 2.40E−01 | 2.45E−01 | 2 | Unchanged Medium | |
| Q9UBK9 | I15cd4 | ubiquitously-expressed transcript | 936.2619 1060 | 1384.764 | 1125.743 | 5.65E−01 | 1.74E−01 | 2 | Unchanged Medium | | |
| O60512 | E11ab3 | UDP-Gal:betaGlcNAc beta 1;4- | 436.7634 | 610 | 542.39 | 529.5839 | 3.12E−01 | 4.81E−01 | 2 | Unchanged Medium | |
| P78383 | P20cd5 | UDP-galactose transporter relate | 1570.135 | 1700 | 2180.117 | 1616.963 | 4.74E−01 | 1.15E−01 | 2 | Unchanged Medium | |
| Q16851 | I24cd4 | UDP-glucose pyrophosphorylase | 1324.08 | 1260 | 1318.78 | 1302.468 | −5.79E−03 | −6.64E−02 | 2 | Unchanged Medium | |
| Q9NZ45 | L02gh3 | uncharacterized hematopoletic s | 652.0685 | 670 | 696.5692 | 672.9895 | 9.52E−02 | 3.98E−02 | 2 | Unchanged Medium | |
| Q9NZ32 | L20gh4 | uncharacterized hypothalamus p | 1035.575 | 1250 | 1354.231 | 1214.412 | 3.87E−01 | 2.75E−01 | 2 | Unchanged Medium | |
| Q9NZ29 | L16gh4 | uncharacterized hypothalamus p | 659.53 | 992 | 1118.776 | 923.518 | 7.62E−01 | 5.89E−01 | 2 | Unchanged Medium | |
| Q15853 | C02cd3 | upstream transcription factor 2; c | 601.6839 | 635 | 698.0128 | 644.9023 | 2.14E−01 | 7.78E−02 | 2 | Unchanged Medium | |
| Q92528 | D02ef7 | uridine monophosphate kinase | 560.3976 | 685 | 623.5243 | 623.0076 | 1.54E−01 | 2.90E−01 | 2 | Unchanged Medium | |
| P15692 | M04ef7 | vascular endothetial growth facto | 460.9257 | 539 | 857.3853 | 619.202 | 8.95E−01 | 2.27E−01 | 2 | Unchanged Medium | |
| P50552 | C08cd3 | vasodilator-stimulated phosphop | 468.4573 | 648 | 392.5569 | 502.9122 | −2.55E−01 | 4.67E−01 | 2 | Unchanged Medium | |
| O60763 | H16cd3 | vesicle docking protein p115 | 639.538 | 543 | 745.4806 | 642.7624 | 2.21E−01 | −2.35E−01 | 2 | Unchanged Medium | |
| Q15836 | A15ef5 | vesicle-associated membrane pr | 804.2648 | 1370 | 1060.211 | 1076.578 | 3.99E−01 | 7.63E−01 | 2 | Unchanged Medium | |
| Q9UEU0 | O22cd6 | vesicle-associated soluble NSF | 468.6024 | 697 | 698.3534 | 621.1953 | 5.76E−01 | 5.72E−01 | 2 | Unchanged Medium | |
| P14921 | E24ef5 | v-ets erythroblastosis virus E26 | 913.893 | 765 | 291.1538 | 656.6528 | −1.65E+00 | −2.57E−01 | 2 | Unchanged Medium | |
| P18206 | C14cd3 | vinculin | 580.3315 | 857 | 865.7505 | 767.5496 | 5.77E−01 | 5.62E−01 | 2 | Unchanged Medium | |
| P00540 | G08ef5 | v-mos Moloney murine sarcoma | 1115.145 | 728 | 663.6747 | 835.5051 | −7.49E−01 | −6.16E−01 | 2 | Unchanged Medium | |
| P01106 | E18ef5 | v-myc myelocytomatosis viral on | 537.8131 | 601 | 405.1047 | 514.7639 | −4.09E−01 | 1.61E−01 | 2 | Unchanged Medium | |
| P21796 | C16cd3 | voltage-dependent anion channe | 483.8969 | 510 | 584.201 | 525.9036 | 2.72E−01 | 7.47E−02 | 2 | Unchanged Medium | |
| Q15765 | C12cd3 | von Hippel-Lindau binding protei | 689.743 | 705 | 891.2621 | 762.0788 | 3.70E−01 | 3.20E−02 | 2 | Unchanged Medium | |
| P10398 | D01ab4 | v-raf murine sarcoma 3611 viral | 1061.812 | 639 | 532.2153 | 744.4583 | −9.96E−01 | −7.32E−01 | 2 | Unchanged Medium | |
| P11233 | A19ef6 | v-ral simian leukemia viral oncog | 848.7099 | 1060 | 1081.428 | 995.8044 | 3.50E−01 | 3.17E−01 | 2 | Unchanged Medium | |
| Q9Y6W5 | A22gh1 | WAS protein family: member 2 | 1283.077 | 1630 | 1493.145 | 1468.514 | 2.19E−01 | 3.45E−01 | 2 | Unchanged Medium | |
| Q9UPY6 | L06cd6 | WAS protein family: member 3 | 1503.584 | 1210 | 2151.643 | 1623.108 | 5.17E−01 | −3.09E−01 | 2 | Unchanged Medium | |
| O75083 | D15cd5 | WD repeat domain 1 | 892.4425 | 977 | 963.3448 | 944.1492 | 1.10E−01 | 1.30E−01 | 2 | Unchanged Medium | |
| O76071 | A24cd5 | WD40 protein Ciao1 | 609.8821 | 896 | 730.5311 | 746.1814 | 2.60E−01 | 5.58E−01 | 2 | Unchanged Medium | |
| Q9HCN4 | H11gh6 | XPA binding protein 1; putative A | 1199.21 | 1070 | 507.3277 | 923.9753 | −1.24E+00 | −1.71E−01 | 2 | Unchanged Medium | |
| Q9UM05 | N17ef3 | yeast Sec31p homolog | 1887.447 | 1590 | 1542.778 | 1672.59 | −2.91E−01 | −2.50E−01 | 2 | Unchanged Medium | |
| Q9UQR1 | P06gh1 | zinc finger protein 148 (pHZ-52) | 513.8639 | 737 | 563.7625 | 604.9201 | 1.34E−01 | 5.21E−01 | 2 | Unchanged Medium | |
| Q14119 | G21ef5 | zinc finger protein 161 | 1199.558 | 1760 | 1197.34 | 1387.253 | −2.67E−03 | 5.57E−01 | 2 | Unchanged Medium | |
| Q9NW07 | F06gh2 | zinc finger protein 358 | 402.8342 | 575 | 684.7069 | 554.3431 | 7.65E−01 | 5.15E−01 | 2 | Unchanged Medium | |
| P20694 | D04ef6 | zinc finger protein 9 (a cellular re | 1042.65 | 1010 | 856.5924 | 969.9823 | −2.84E−01 | −4.49E−02 | 2 | Unchanged Medium | |

What is claimed is:

1. A method for transforming IP cells that express markers of acinar cells and liver-associated genes into insulin-producing cells in vitro, comprising culturing said IP cells in a cell culture medium comprising an effective amount of at least one differentiation promoting factor selected from the group consisting of C-Natriuretic Peptide (CNP), Calcitonin Gene Related Peptide, Cholera Toxin B Subunit, Dexamethasone, Gastrin-Releasing Peptide, Laminin, Met-Enkephalin, PDGFAA+PDGFBB, Sonic Hedgehog, and Substance P such that the IP cells are transformed into insulin-producing cells.

2. The method of claim 1, wherein the IP cells are derived from a culture of pancreatic acinar cells.

3. The method of claim 2, wherein the cells are human.

4. The method of claim 1, further comprising contacting said cells with a substrate that is coated with one or more extracellular matrix molecules.

5. The method of claim 4, wherein the extracellular matrix molecules are collagen I, collagen VI, collagen IV, vitronectin, and/or fibronectin.

6. The method of claim 4, wherein the substrate is on the surface of a flask, petri dish, plate, well or roller bottle, or is part of a scaffold.

7. The method of claim 1, wherein the medium is serum-free.

8. The method of claim 1, wherein the medium comprises serum.

9. The method of claim 7, wherein the medium comprises BSA, insulin, transferrin, selenium and epidermal growth factor (EGF).

10. The method of claim 3, wherein the cells are seeded on the substrate at a density of 5×10³ to 20×10⁵ cells/cm².

11. An isolated insulin-producing cell generated by the method of claim 1.

12. An insulin-producing cell, prepared by differentiating a mammalian acinar cell in vitro, wherein said insulin-producing cell has an expression profile after 16 days ex vivo as shown in Table 6.

13. A serum-free medium comprising at least one differentiation promoting factor selected from the group consisting of C-Natriuretic Peptide (CNP), Calcitonin Gene Related Peptide, Cholera Toxin B Subunit, Dexamethasone, Gastrin-Releasing Peptide, Laminin, Met-Enkephalin, PDGFAA+PDGFBB, Sonic Hedgehog, and Substance P wherein said medium facilitates differentiation of IP cells into insulin-producing cells.

14. A serum free medium comprising a 1:1 mixture of DMEM and Hams F12 plus the components listed in Table 2.

15. A kit suitable for differentiating IP cells to insulin-producing cells, comprising
a) a base medium suitable for the cultivation of mammalian epithelial cells;
b) a collagen I coated culture substrate, and, separately packaged,
c) a serum-free medium supplement containing BSA, C-Natriuretic Peptide (CNP), Calcitonin Gene Related Peptide, Cholera Toxin B Subunit, Dexamethasone, Gastrin-Releasing Peptide, Laminin, Met-Enkephalin, PDGFAA+PDGFBB, Sonic Hedgehog, and Substance P or two or more of these components in combination, in suitable amounts to yield final concentrations in the completed medium as indicated in Table 1 herein.

16. The kit of claim 15, wherein the cell culture substrate is contained on the surface of a flask, bottle, petri dish, plate or well suitable for cell culture.

17. The method of claim 1, wherein the cell culture medium comprises a 1: 1 mixture of DMEM and Hams 12.

18. The serum-free medium of claim 13 which comprises a 1:1 mixture of DMEM and Hams 12.

19. The method of claim 1, wherein said differentiation promoting factors have the concentrations in the medium as indicated in Table 1.

20. The serum-free medium of claim 13, wherein said differentiation promoting factors have the concentrations in the medium as indicated in Table 1.
